(19) United States
(12) Patent Application Publication
(10) Pub. No.: US 2023/0322761 A1
(43) Pub. Date: Oct. 12, 2023

(54) DEGRADATION OF BRUTON'S TYROSINE KINASE (BTK) BY CONJUGATION OF BTK INHIBITORS WITH E3 LIGASE LIGAND AND METHODS OF USE

(71) Applicant: BEIGENE, LTD., Camana Bay, Grand Cayman (KY)

(72) Inventors: Hexiang WANG, Beijing (CN); Bailin LEI, Beijing (CN); Changxin HUO, Beijing (CN); Dongqing SUN, Beijing (CN); Jie CHEN, Beijing (CN); Zhiwei WANG, Beijing (CN)

(21) Appl. No.: 17/910,037

(22) PCT Filed: Mar. 10, 2021
(86) PCT No.: PCT/CN2021/079882

§ 371 Date: Sep. 8, 2022

(30) Foreign Application Priority Data
Mar. 11, 2020 (WO) PCT/CN2020/078884
Feb. 9, 2021 (WO) PCT/CN2021/076294

(51) Int. Cl. C07D 471/04

(52) U.S. Cl. CPC C07D 471/04

(57) ABSTRACT

Disclosed herein are novel bifunctional compounds formed by conjugating BTK inhibitor moieties with E3 ligase Ligand moieties, which function to recruit targeted proteins to E3 ubiquitin ligase for degradation, and methods of preparation and uses thereof.

### FIELD OF THE INVENTION

Disclosed herein are novel bifunctional compounds formed by conjugating BTK inhibitor moieties with E3 ligase Ligand moieties, which function to recruit targeted proteins to E3 ubiquitin ligase for degradation, and methods of preparation and uses thereof.

### BACKGROUND OF THE INVENTION

Proteolysis-targeting chimera (PROTAC) is a novel strategy for selective knockdown of target proteins by small molecules (Sakamoto K M et al., Proc Natl Acad Sci 2001, 98:8554-9.; Sakamoto K. M. et al., Methods Enzymol. 2005; 399:833-847.). PROTAC utilizes the ubiquitin-protease system to target a specific protein and induce its degradation in the cell (Zhou P. et al., Mol Cell. 2000; 6(3):751-756; Neklesa T. K. et al., Pharmacol Ther. 2017; 174:138-144; Lu M. et al., Eur J Med Chem. 2018; 146:251-259;). The normal physiological function of the ubiquitin-protease system is responsible for clearing denatured, mutated, or harmful proteins in cells. The ubiquitin-proteasome system (UPS), also known as the ubiquitin-proteasome pathway (UPP), is a common posttranslational regulation mechanism that is responsible for protein degradation in normal and pathological states (Ardley H. et al., Essays Biochem. 2005, 41, 15-30; Komander D. et al., Biochem. 2012, 81, 203-229; Grice G. L. et al., Cell Rep. 2015, 12, 545-553; Swatek K. N. et al., Cell Res. 2016, 26, 399-422). Ubiquitin, which is highly conserved in eukaryotic cells, is a modifier molecule, composed of 76 amino acids, that covalently binds to and labels target substrates via a cascade of enzymatic reactions involving E1, E2, and E3 enzymes. Subsequently, the modified substrate is recognized by the 26S proteasome complex for ubiquitination-mediated degradation. So far, two E1 enzymes have been discovered, which are termed UBA1 and UBA6. On the other hand, there are about 40 E2 enzymes and more than 600 E3 enzymes that offer the functional diversity to govern the activity of many downstream protein substrates. However, only a limited number of E3 ubiquitin ligases have been successfully hijacked for use by small molecule PROTAC technology: the Von Hippel-Lindau disease tumor suppressor protein (VHL), the Mouse Double Minute 2 homologue (MDM2), the Cellular Inhibitor of Apoptosis (cIAP), and cereblon (Philipp O. et al., Chem. Biol. 2017, 12, 2570-2578).

Bifunctional compounds composed of a target protein-binding moiety and an E3 ubiquitin ligase-binding moiety have been shown to induce proteasome-mediated degradation of selected proteins. These drug-like molecules offer the possibility of temporal control over protein expression, and could be useful as biochemical reagents for the treatment of diseases. In recent years, this newly developed method has been widely used in antitumor studies (Lu J. et al., Chem Biol. 2015; 22(6):755-763; Ottis P. et al., Chem Biol. 2017; 12(4):892-898.; Crews C. M. et al., J Med Chem. 2018; 61(2):403-404; Neklesa T. K. et al., Pharmacol Ther. 2017, 174:138-144.; Cermakova K. et al., Molecules, 2018.23(8).; An S. et al., EBioMedicine, 2018.; Lebraud H. et al., Essays Biochem. 2017; 61(5): 517-527.; Sun Y. H. et al., Cell Res. 2018; 28:779-81; Toure M. et al., Angew Chem Int Ed Engl. 2016; 55(6):1966-1973;); and has been disclosed or discussed in patent publications, e.g., US20160045607, US20170008904, US20180050021, US20180072711, WO2002020740, WO2014108452, WO2016146985, WO2016149668, WO2016149989, WO2016197032, WO2016197114, WO2017011590, WO2017030814, WO2017079267, WO2017182418, WO2017197036, WO2017197046, WO2017197051, WO2017197056, WO2017201449, WO2017211924, WO2018033556, and WO2018071606.

Bruton's tyrosine kinase (Btk) belongs to the Tec tyrosine kinase family (Vetrie et al., Nature 361: 226-233, 1993; Bradshaw, Cell Signal. 22: 1175-84, 2010). Btk is primarily expressed in most hematopoietic cells such as B cells, mast cells and macrophages (Smith et al., J. Immunol. 152: 557-565, 1994) and is localized in bone marrow, spleen and lymph node tissue. Btk plays important roles in B-cell receptor (BCR) and FcR signaling pathways, which involve in B-cell development, differentiation (Khan, Immunol. Res. 23: 147, 2001). Btk is activated by upstream Src-family kinases. Once activated, Btk, in turn, phosphorylates PLC gamma, leading to effects on B-cell function and survival (Humphries et al., J. Biol. Chem. 279: 37651, 2004). These signaling pathways must be precisely regulated. Mutations in the gene encoding Btk cause an inherited B-cell specific immunodeficiency disease in humans, known as X-linked agammaglobulinemia (XLA) (Conley et al., Annu. Rev. Immunol. 27: 199-227, 2009). Aberrant BCR-mediated signaling may result in dysregulated B-cell activation leading to a number of autoimmune and inflammatory diseases. Preclinical studies show that Btk deficient mice are resistant to developing collagen-induced arthritis. Moreover, clinical studies of Rituxan, a CD20 antibody to deplete mature B-cells, reveal the key role of B-cells in a number of inflammatory diseases such as rheumatoid arthritis, systemic lupus erythematosus and multiple sclerosis (Gurcan et al., Int. Immunopharmacol. 9: 10-25, 2009). Therefore, Btk inhibitors can be used to treat autoimmune and/or inflammatory diseases.

Inhibition of BTK has been shown to affect cancer development (B cell malignancies) and cell viability, and improve autoimmune diseases (e.g., rheumatoid arthritis and lupus). Inhibition of BTK has also been reported via alternative strategies, such as through degradation of BTK (Alexandru D. et al., Biochemistry 2018, 57, 26, 3564-3575; Adelajda Z. et al., PNAS 2018 115 (31); Dennis D., et al., Blood, 2019, 133:952-961; Yonghui S. et al., Cell Research, 2018, 28, 779-781; Yonghui S. et al., Leukemia, 2019, Degradation of Bruton's tyrosine kinase mutants by PROTACs for the potential treatment of ibrutinib-resistant non-Hodgkin lymphomas).

There is a need for new BTK inhibitors which are more potent than known inhibitors of BTK and inhibit BTK via alternative strategies, such as through degradation of BTK. The present application addresses the need.

There is no literature reported 3,5-disubstituted-1H-pyrazolo[3,4-b] pyridine can be new BTK inhibitors. This application firstly describes that 3,5-disubstituted-1H-pyrazolo[3,4-b]pyridine can be a good BTK inhibitor, and can be used as PROTAC-derived degrader for BTK degradation.

### SUMMARY OF THE INVENTION

One objective of the present invention is to provide a proteolysis targeting chimera (PROTAC) compound by conjugating a BTK inhibitor with an E3 ligase ligand, which functions to recruit targeted proteins to E3 ubiquitin ligase for degradation, and to provide a method of the preparation and uses thereof. In particular, the present disclosure provides PROTAC compounds with the Formula I.

Aspect 1: A compound of Formula (I):

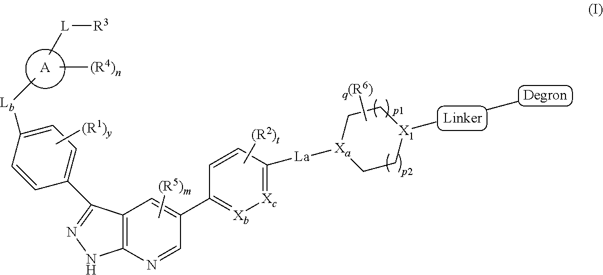

or a pharmaceutically acceptable salt thereof, or a stereoisomer thereof,
wherein:
A is a 5- or 6-membered aromatic ring comprising 0-3 heteroatoms selected from nitrogen, oxygen and sulfur;
X₁ and Xₐ are each selected from —CH— or N;
X_{b} and X_{c} are each selected from —CR^{a}— or N;
L and L_{b}, are each independently a bond, —(CR^{a}R^{b})ᵤ₁—, —NR⁷—, —O—, —S—, —(CR^{a}R^{b})ᵤ₁—NR⁷—C(O)—, —C(O)—NR⁷—(CR^{a}R^{b})ᵤ₁—, and Lₐ is a bond, —(CR^{a}R^{b})ᵤ₁—, —NR⁷—, —O—, —S—, —(CR^{a}R^{b})ᵤ₁—NR⁷—C(O)—, —C(O)—NR⁷—(CR^{a}R^{b})ᵤ₁—,

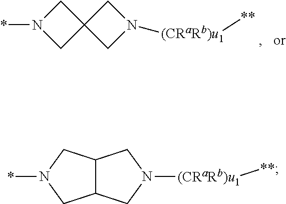

wherein u1 is an integral of 0-12; wherein * refers to the position attached to the

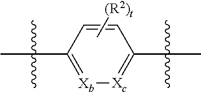

moiety, and ** refers to the position attached to the

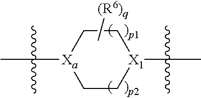

moiety;
t, m, n, q, and y are each independently 0, 1, 2, 3 or 4;
p1 and p2 are each independently 0, 1 or 2;
R⁷ is each independently hydrogen, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
R¹, R², R³, R⁴, R⁵, and R⁶ are each independently hydrogen, halogen, —C₁₋₈alkyl, —C₂₋₈alkenyl, —C₂₋₈alkynyl, cycloalkyl, heterocyclyl, aryl, heteroaryl, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, hydroxyl-C₁₋₈alkyl-, -haloC₁₋₈alkyl, —C₁₋₈alkyoxy, cycloalkyl, heterocyclyl, aryl, or heteroaryl;
or in the case that two substituent R⁶ are substituted on the

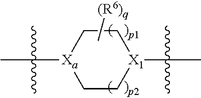

moiety, two R⁶ together with the remainder of the moiety form a fused ring or a bridged ring wherein the bridge comprises one, two, three or four —CH₂— moieties in addition to the two bridgeheads;
R^{a}, R^{b}, and R^{c} are each independently hydrogen, —C₁₋₈alkyl, —C₂₋₈alkenyl, —C₂₋₈alkynyl, cycloalkyl, heterocyclyl, aryl, or heteroaryl;
or R^{a} and R^{b}, together with the nitrogen atom to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{3f}, —SO₂R^{3f}, —SO₂NR^{3f}R^{3g}, —COR^{3f}, —CO₂R^{3f}, —CONR^{3f}R^{3g}, —C(═NR^{3f})NR^{3g}R^{3h}, —NR^{3f}R^{3g}, —NR^{3f}COR^{3g}, —NR^{3f}CONR^{3g}R^{3h}, —NR^{3f}CO₂R^{3g}, —NR^{3f}SONR^{3g}R^{3h}, —NR^{3f}SO₂NR^{3g}R^{3h}, or —NR^{3f}SO₂R^{3g}, each of said —C₁₋₈alkyl, —C₂₋₈alkenyl, —C₂₋₈alkynyl, cycloalkyl, heterocyclyl, aryl, or heteroaryl is optionally substituted with at least one substituent selected from halogen, —C₁₋₈alkyl, —OR³ⁱ, —NR³ⁱR^{3j}, cycloalkyl, heterocyclyl, aryl, or heteroaryl;
R^{3f}, R^{3g}, R^{3h}, R³ⁱ, and R^{3j} are each independently hydrogen, —C₁₋₈alkyl, C₁₋₈alkoxy-C₁₋₈alkyl-, —C₂₋₈alkenyl, —C₂₋₈alkynyl, cycloalkyl, heterocyclyl, aryl, or heteroaryl;
the Linker is a bond or a divalent linking group, and
the Degron moiety is an E3 Ubiquitin ligase moiety.

Aspect 2: The compound according to Aspect 1, wherein the Degron moiety is selected from Formulas D1, D2, D3, D4, D5, D6, D7 or D8:

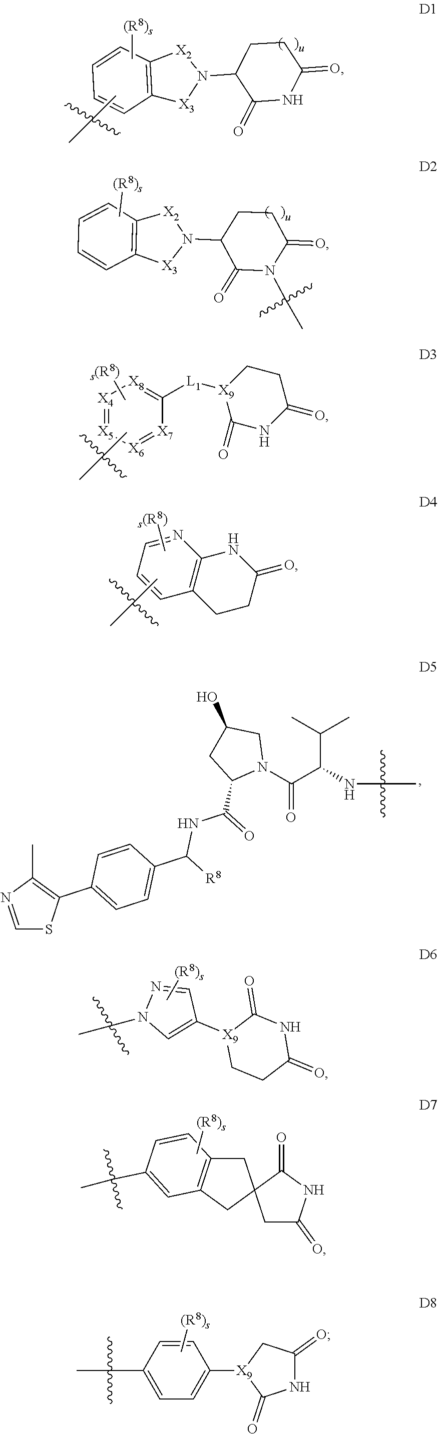

wherein
X₂ and X₃ are each independently —CH₂—, —NH— or —C(O)—;
X₄, X₅, X₆, X₇ and X₈ are each independently CH or N;
X₉ is CH or N;
L₁ is selected from a bond, —CH₂—, —O—, —NH— and —S—;
s is 0, 1, 2, 3, or 4;
u is 0, 1, or 2;
R⁸ is each independently hydrogen, halogen, —C₁₋₈alkyl, —C₂₋₈alkenyl, —C₂₋₈alkynyl, cycloalkyl, heterocyclyl, aryl, heteroaryl, —CN, —NO₂, —OR^{8a}, —SO₂R^{8a}, —COR^{8a}, —CO₂R^{8a}, —CONR^{8a}R^{8b}, —C(═NR^{8a})NR^{8b}R^{8c}, —NR^{8a}R^{8b}, —NR^{8a} COR^{8b}, —NR^{8a} CONR^{8b}R^{8c}, —NR^{8a} CO₂R^{8b}, —NR^{8a} SONR^{8b}R^{8c}, —NR^{8a} SO₂NR^{8b}R^{8c}, or —NR^{8a}SO₂R^{8b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
R^{8a}, R^{8b}, and R^{8c} are each independently hydrogen, —C₁₋₈alkyl, —C₂₋₈alkenyl, —C₂₋₈alkynyl, cycloalkyl, heterocyclyl, aryl, or heteroaryl;
Alternatively, two adjacent R⁸, together with the ring to which they are attached, form a fused ring;
wherein the Degron moiety binds to the linker via

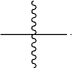

Aspect 3: The compound according to Aspect 1, wherein Formula D1 is selected from

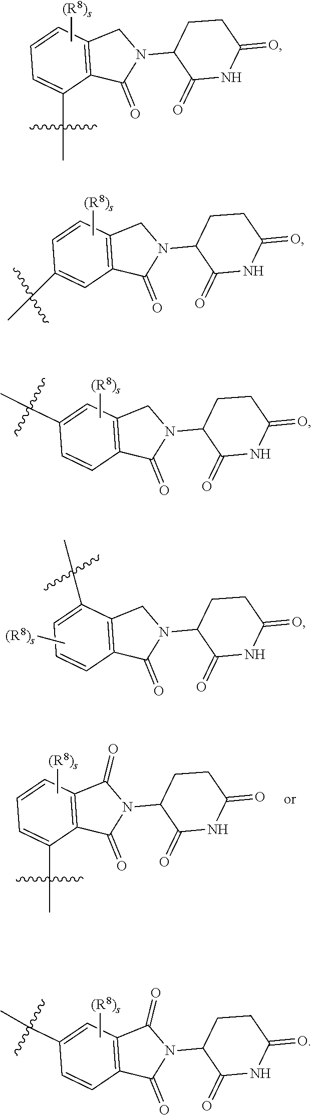

Aspect 4: The compound according to Aspect 14 wherein Formula D1 or D2 is selected from

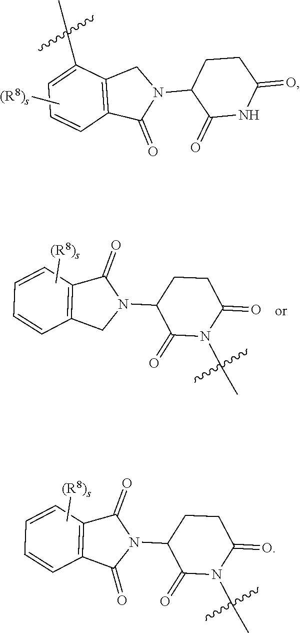

Aspect 5: The compound according to Aspect 2, wherein Formula D1 D or D2 is selected from

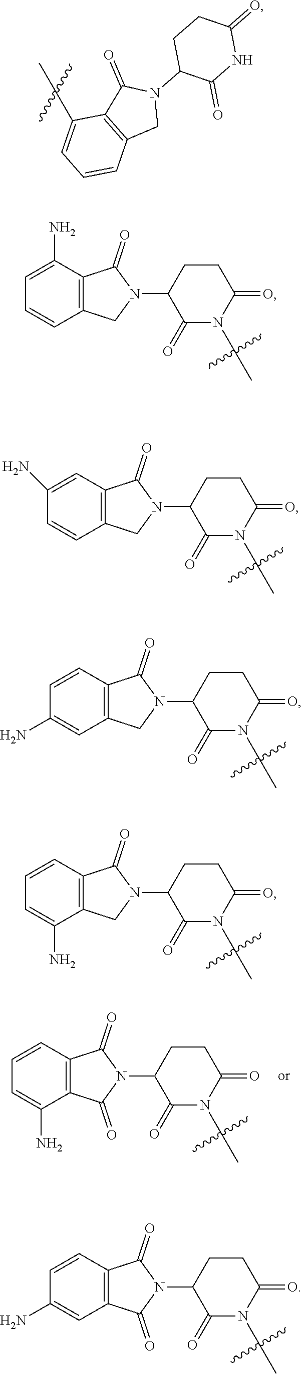

Aspect 6: The compound according to Aspect 2, wherein Formula D3, D6 or D8 is selected from

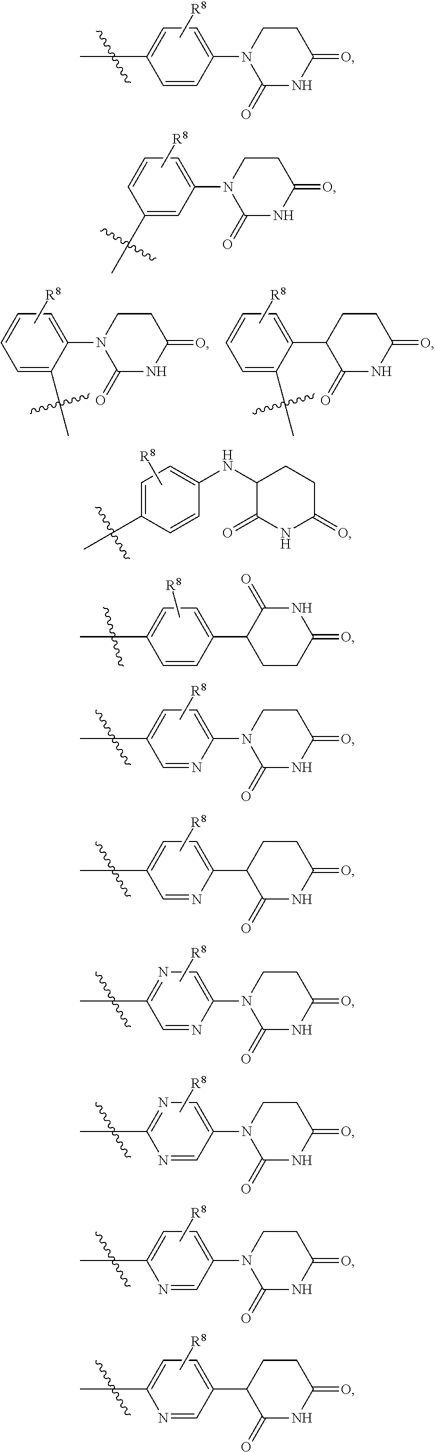

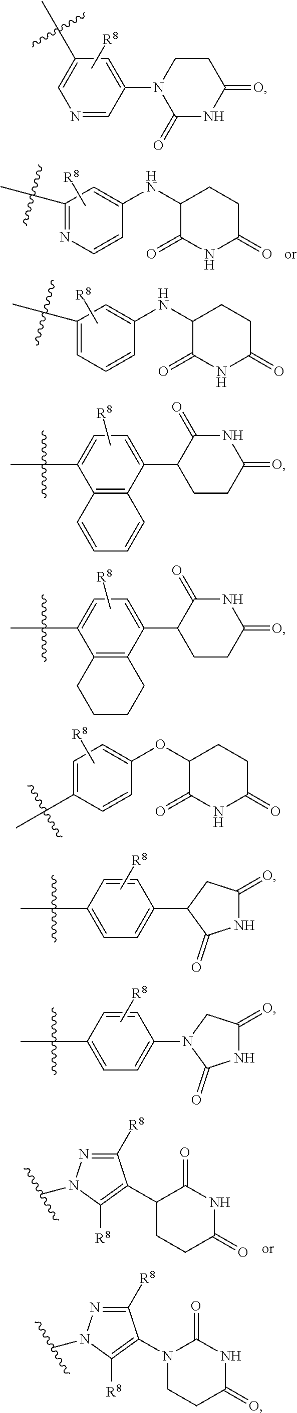

wherein R⁸ is defined as above.

Aspect 7: The compound according to Aspect 6, wherein Formula D3, D6 or D8 is selected from

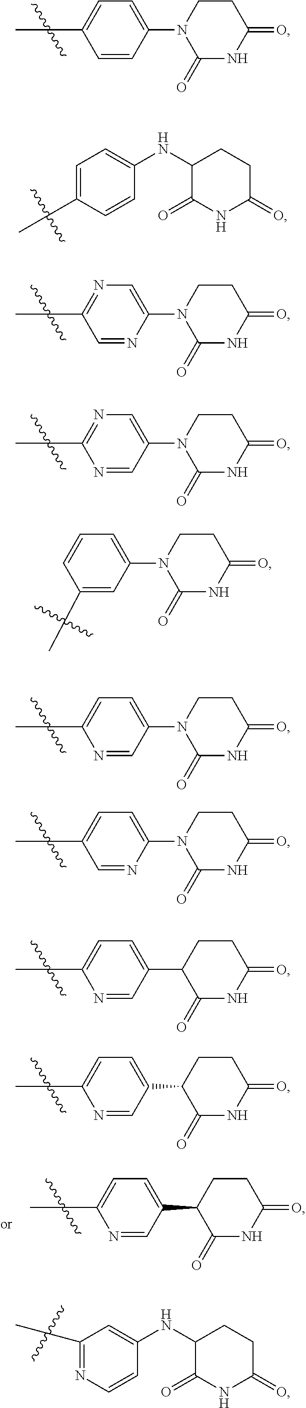

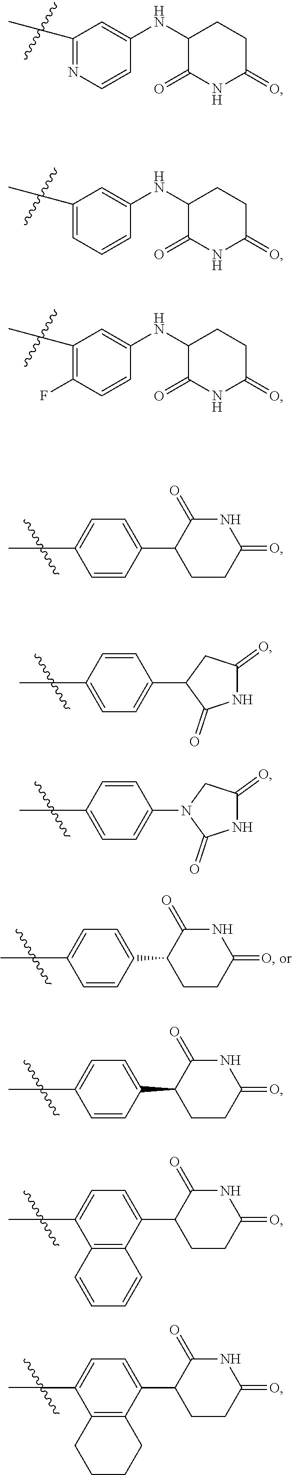

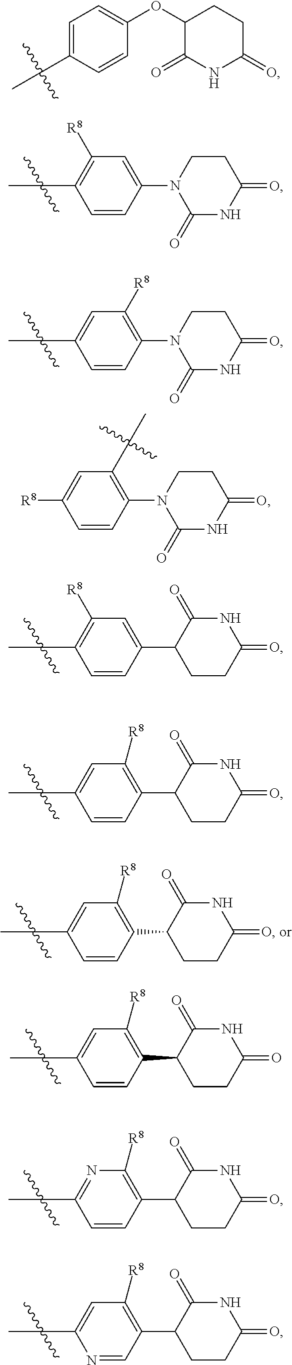

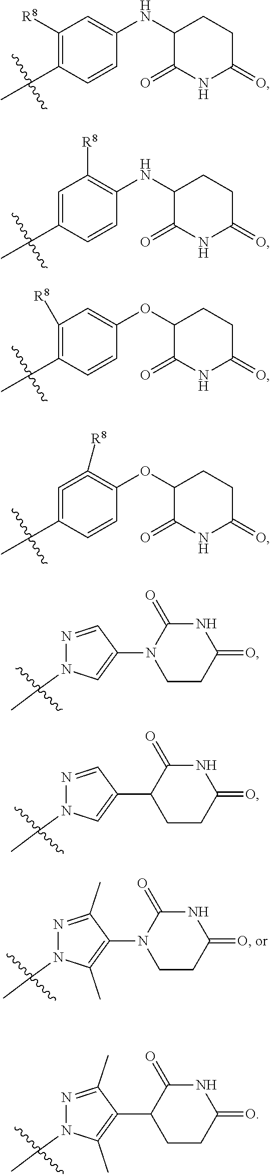

Wherein R⁸ is halogen, —C₁₋₈alkyl, or —C₁₋₈alkoxy; preferably fluoro, chloro, methyl or methoxy.

Aspect 8: The compound according to Aspect 2, wherein Formula D4 is selected from

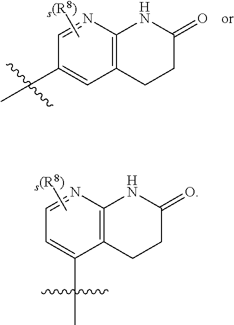

Aspect 9: The compound according to Aspect 8, wherein Formula D4 is selected from

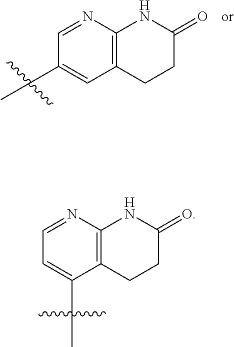 [SMILES: *c1cnc2c(c1)CCC(=O)N2 ; *c1ccnc2c1CCC(=O)N2]

Aspect 10: The compound according to any one of Aspects 1-9, where in the Linker is selected from a bond,

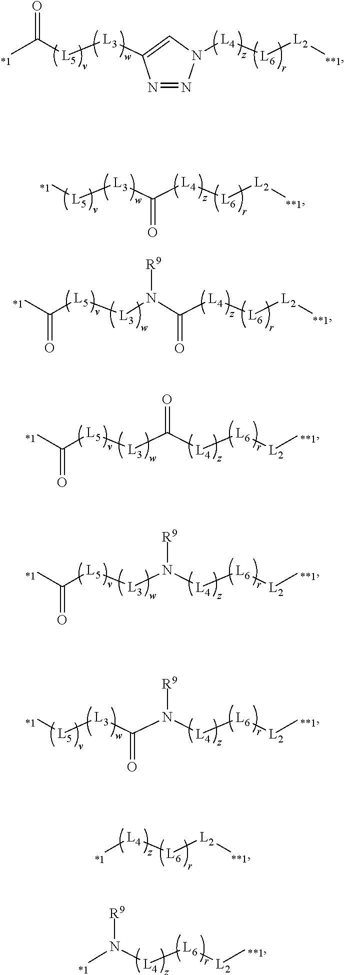

wherein *1 refers to the position attached to the

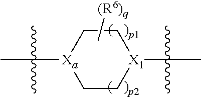

moiety, and **1 refers to the position attached to the Degron;
r, v, w, and z are each independently 0, 1, 2, 3, 4, 5, 6, 7, 8, 9 or 10;
L₂ is —CH₂—, —NH—, O—, —C(O)—, —NHC(O)—,

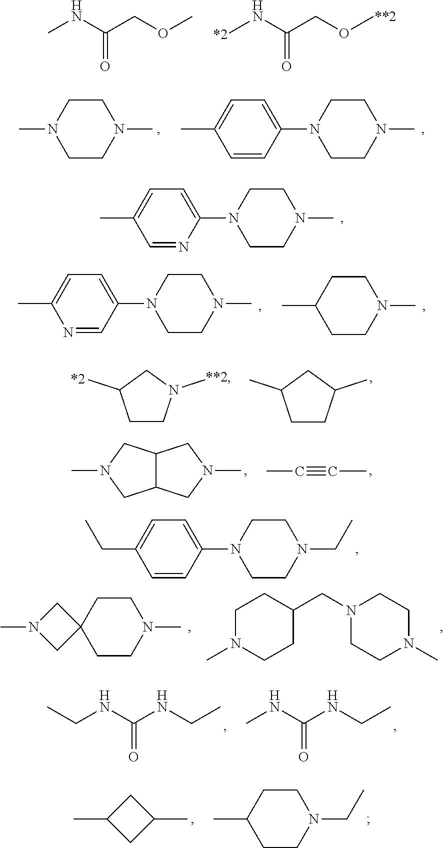

wherein *2 refers to the position attached to L₄ and **2 refers to the position attached to the Degron;
L₃, L₄, L₅ and L₆ are each independently —CH₂—, —CH₂—CH(CH₃)—, —CF₂—, —CH₂CH—, —OCH₂CH₂—, —CH₂—O—CH₂—, —CH₂CH₂O—, —C(O)—, —NHC(O)—, —CH₂—CONH—,

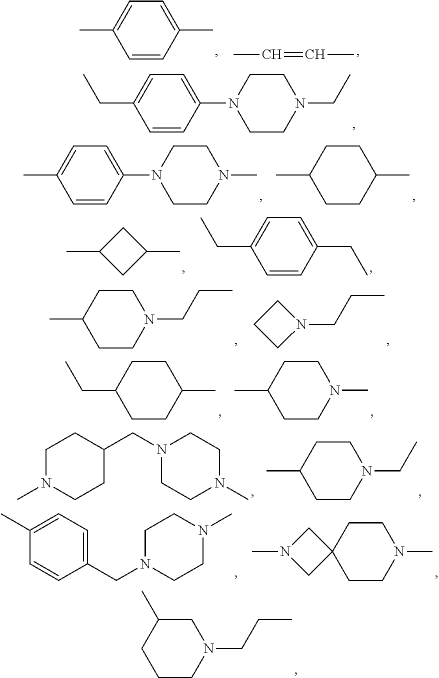

R⁹ is selected from H or CH₃.

Aspect 11: The compound according to any one of Aspects 1-10, wherein the Linker is selected from

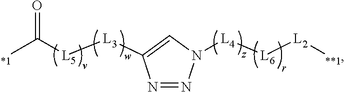

v=0; w=0, 1, 2, 3, or 4; L₃ is —CH₂—; L₄ is —CH₂CH₂O— or —CH₂—; z=0, 1, 2, 3, 4, 5, 6, or 7; L₆ is —CH₂— or —NHC(O)—; r=0, 1, 2, 3, or 4; L₂ is —NH—, —CH₂—, —O— or —C≡C—.

Aspect 12: The compound according to Aspect 10, wherein v=0; w=0; L₄ is —CH₂CH₂O—; z=1, 2, 3, 4, 5, 6, or 7; L₆ is —CH₂—; r=0, 1, 2, or 3; L₂ is —NH—, or —CH₂—.

Aspect 13: The compound according to Aspect 11, wherein v=0; w=0; L₄ is —CH₂CH₂O—; z=1, 2, 3; L₆ is —CH₂—; r=1, 2, or 3; L₂ is —C≡C—.

Aspect 14: The compound according to Aspect 10, wherein v=0, L₃ is —CH₂—, w=2 or 3, L₄ is —CH₂CH₂O— or —CH₂—, z=1, 2, 3, or 4; L₆ is —CH₂—; r=1, 2, or 3; L₂ is —NH—, or —CH₂—.

Aspect 15: The compound according to Aspect 10, wherein v=0, L₃ is —CH₂—, w=2 or 3, L₄ is —CH₂—, z=3, 4 or 5; r=0; L₂ is

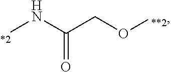

wherein *2 refers to the position attached to L₄ and **2 refers to the position attached to the Degron.

Aspect 16: The compound according to Aspect 10, wherein the Linker is selected from

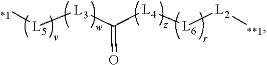

wherein
L₅ is —CH₂CH₂O—, or

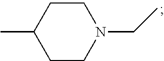

v=0, 1, 2 or 3, L₃ is —CH₂— or

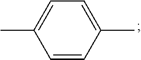

w=0, 1, 2, or 3; L₄ is —CH₂—O—CH₂—, —CH₂—,

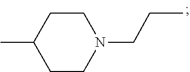

z=0, 1, 2, 3, 4, 5 or 6; L₆ is —CH₂—, —OCH₂CH₂—,

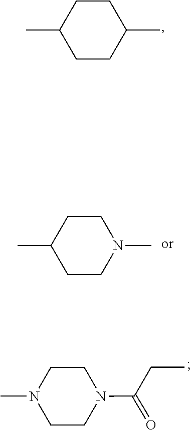

r=0, 1, 2, 3, 4, 5, 6, 7 or 8; L₂ is —NH—,

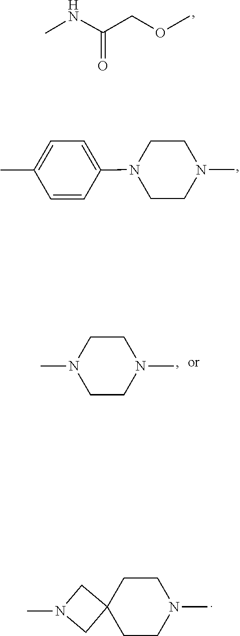

Aspect 17: The compound according to Aspect 16, wherein L₅ is —CH₂CH₂O—; v=1, 2 or 3, L₃ is —CH₂—; w=1; z=0; r=0; L₂ is —NH—.

Aspect 18: The compound according to Aspect 16, wherein v=w=0; L₄ is —CH₂—O—CH₂—; z=1, 2, 3 or 4; L₆ is —CH₂—; r=1, 2, 3, 4, 5, 6, 7 or 8; L₂ is —NH— or

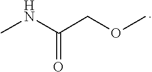

Aspect 19: The compound according to Aspect 16, wherein v=w=z=0; L₆ is —CH₂—; r=2, 3, 4, 5, or 6;
L₂ is —NH— or

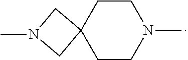

Aspect 20: The compound according to Aspect 16, wherein v=w=0; L₄ is

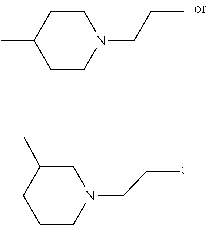

z=1; L₆ is —OCH₂CH₂—; r=1, 2, 3; L₂ is —NH—.

Aspect 21: The compound according to Aspect 16, wherein the Linker is selected from

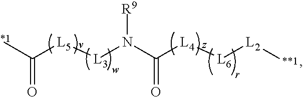

wherein
L₅ is —CH₂CH₂O—, —CH₂— or —CH₂—O—CH₂—;
v=1, 2, 3 or 4, L₃ is —CH₂—,

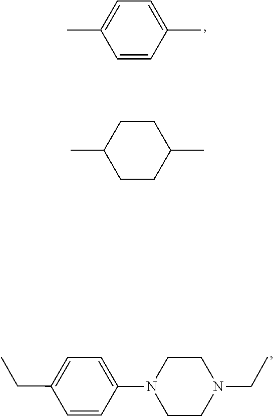

or —CH₂CH₂O—;
w=0, 1, 2 or 3;
R⁹ is H or CH₃;
L₄ is —CH₂— or —CH₂—O—CH₂—;
z=0, 1, 2, 3, or 4; L₆ is —CH₂—;
r=0, 1, 2, 3, or 4; L₂ is —NH—, —CH₂—, —O—,

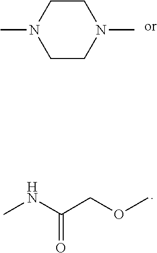

Aspect 22: The compound according to Aspect 21, wherein
L₅ is —CH₂—;
v=1, 2, 3 or 4,
L₃ is

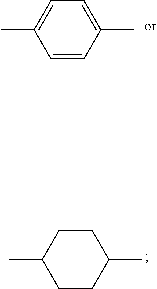

w=1;
R⁹ is H;
L₄ is —CH₂—;
z=1; r=0; L₂ is —NH—.

Aspect 23: The compound according to Aspect 10, wherein the Linker is selected from

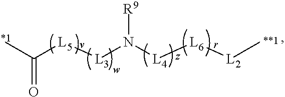

wherein
L₅ is —CH₂CH₂O—, —CH₂—, —CH═CH—, or —CH₂—O—CH₂—;
v=1, 2, 3 or 4, L₃ is —CH₂—, —C(O)—

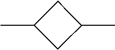

or —CH₂CH₂O—;
w=0, 1, 2 or 3; R⁹ is H or CH₃; L₄ is —CH₂CH₂O—, —CH₂—, —CH₂—O—CH₂—, —CH₂—CONH— or

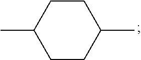

z=0, 1, 2, 3, 4 or 5; L₆ is —CH₂—,

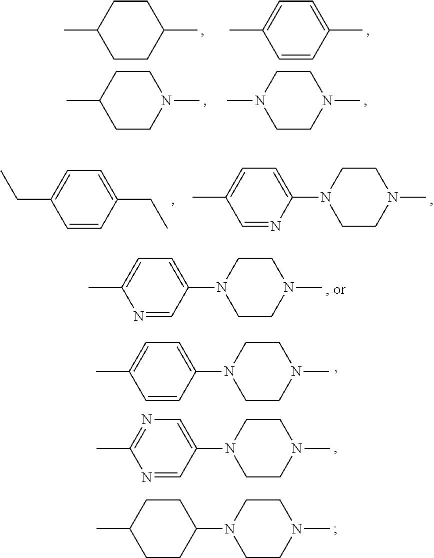

r=0, 1, 2, 3, 4, 5, or 6; L₂ is —NH—, —C(O)—, —O—,

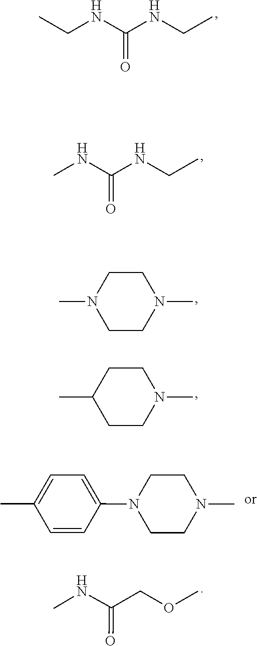

Aspect 24: the compound according to Aspect 23, wherein L₅ is —CH₂—; v=2; w=0; R⁹ is CH₃; L₄ is —CH₂—; z=1, 2, 3, or 4; L₆ is —CH₂—,

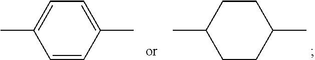

r=0, 1 or 2; L₂ is —NH—,

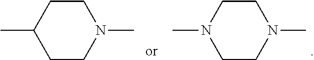

Aspect 25: the compound according to Aspect 23, wherein L₅ is —CH═CH—; v=1, L₃ is —CH₂—; w=0 or 1; R⁹ is H or CH₃; L₄ is —CH₂CH₂O— or —CH₂—; z=1, 2, 3, 4, 5 or 6; L₆ is —CH₂—; r=0, 1, 2; L₂ is —NH—, —CH₂—, or

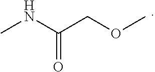

Aspect 26: the compound according to Aspect 23, wherein v=0;
L₃ is

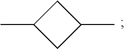

w=1;
R⁹ is CH₃;
L₄ is —CH₂—;
z=1 or 2;
L₆ is

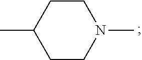

r=0 or 1;
L₂ is —NH—,

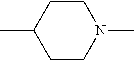

or —C(O)—.

Aspect 27: the compound according to Aspect 10, wherein the Linker is selected from

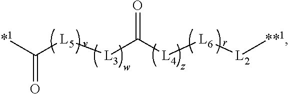

wherein
L₅ is —CH═CH—;
v=1, 2, 3 or 4;
L₃ is

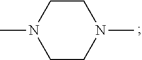

w=1;
L₄ is —CH₂—;
z=1, 2;
L₆ is —CH₂—;
r=0;
L₂ is —NH— or —CH₂—.

Aspect 28: the compound according to Aspect 10, wherein the Linker is selected from

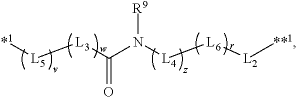

wherein
L₅ is

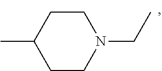

CH₂CH₂O—, —CH₂— or —CH₂—O—CH₂—;
v=1, 2, 3 or 4,
L₃ is —CH₂—,

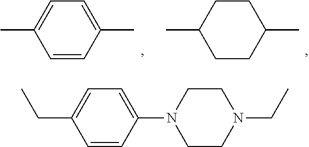

or —CH₂CH₂O—;
w=0, 1, 2 or 3;
R⁹ is H or CH₃;
L₄ is —CH₂—, —CH₂—O—CH₂—,

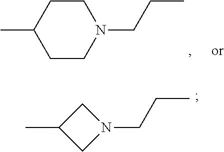

z=0, 1, 2, 3, or 4;
L₆ is —CH₂— or —OCH₂CH₂—;
r=0, 1, 2, 3, or 4;
L₂ is —NH—, —CH₂—, —O—,

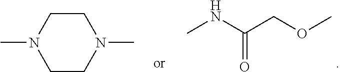

Aspect 29: the compound according to Aspect 10, wherein the Linker is selected from

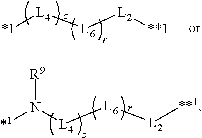

L₄ is —CH₂—, —CF₂—,

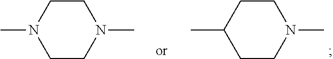

z=0, 1, 2, 3, 4, 5 or 6;
L₆ is —CH₂—, —CF₂—, —CHCH₃—,

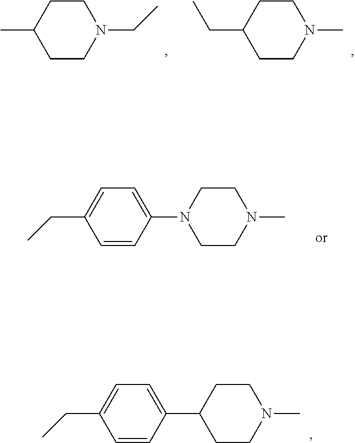

r=0 or 1;
L₂ is —O—, —C(O)—,

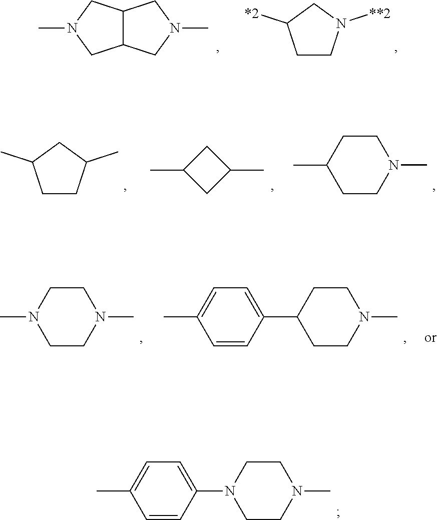

R⁹ is H or CH₃.

In some embodiment, the Linker is

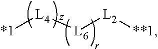

wherein L₄ is —CH₂— or —CF₂—; z=0, 1, 2, 3, 4, 5 or 6; r=0; and L₂ is —O—, —C(O)—,

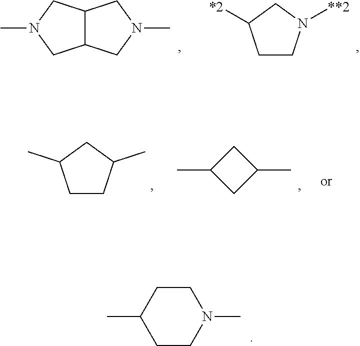

In some embodiment, the Linker is —CH₂-piperidin-4-yl, —CH₂—CH₂—CH₂—O—, —CH₂CH₂—, a bond, —CH₂CH₂-piperidin-4-yl, or —C(O)—.

In a preferred embodiment,

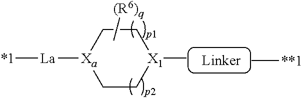

is selected from

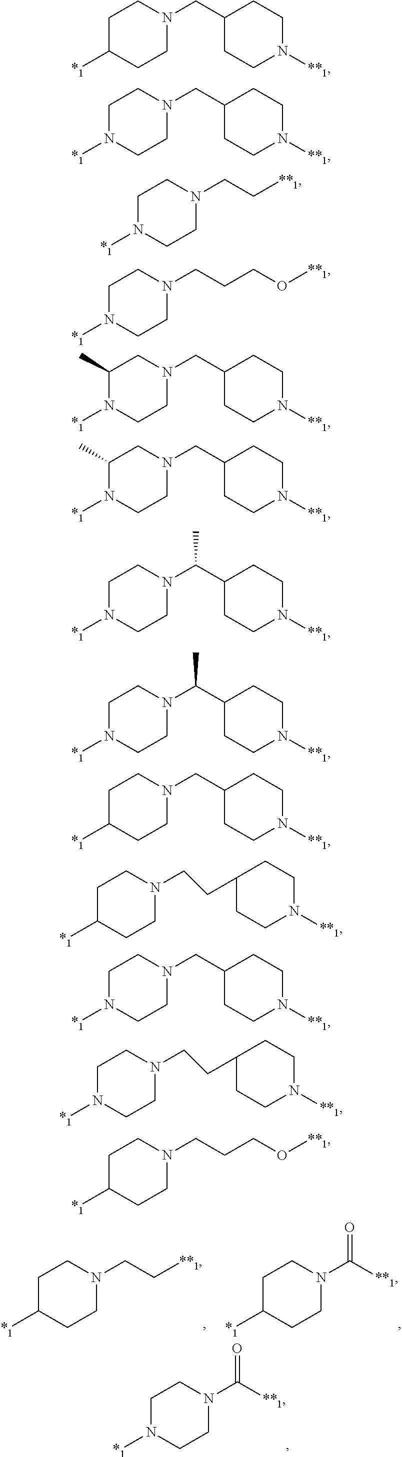

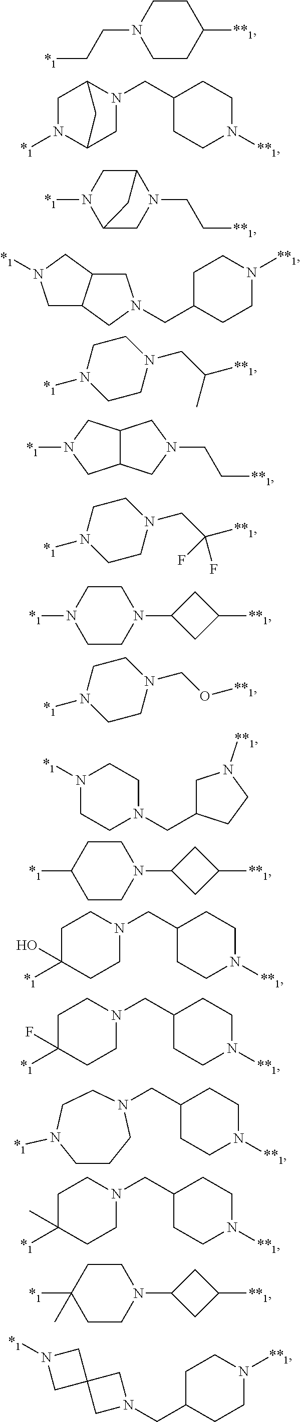

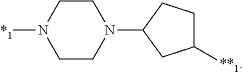

Aspect 31: The compound according to Aspect 1, wherein ring A is 5-membered aromatic ring comprising 1-3 heteroatoms selected from nitrogen and oxygen. In some embodiment, ring A is benzyl, oxadiazole, triazole, thiazole, or pyrazole, preferably, 1,2,4-oxadiazole-3-yl, 1,2,4-oxadiazole-5-yl, 1H-1,2,3-triazole-4-yl, or 1H-pyrazol-4-yl. In some embodiment, n is zero or one, L is a bond, and R³ is —C₁₋₈alkyl or cycloalkyl, each optionally substituted with C₁₋₈alkyl, halogen, hydroxyl-C₁₋₈alkyl-, or -haloC₁₋₈alkyl. In some embodiment, n is zero, L is a bond, and R³ is C₃₋₄alkyl or C₃₋₆cyclopropyl, optionally substituted with halo, —CH₂OH or -haloC₁₋₄alkyl; preferably R₃ is tertbutyl, 1,1,1-trifluoro-2-methylpropan-2-yl, 1-(trifluoromethyl)cyclopropyl, 1-methylcyclopropyl,2-hydroxyprapan-2-yl or 1-hydroxymethylcyclopropyl. In some embodiment, ring A is 5-tertbutyl-1,2,4-oxadiazole-3-yl, 3-tert butyl-1,2,4-oxadiazole-5-yl, 1-tertbutyl-1H-1,2,3-triazole-4-yl, 1-tertbutyl-1H-pyrazol-4-yl, 5-(1,1,1-trifluoro-2-methylpropan-2-yl)-1,2,4-oxadiazole-3-yl, or 5-(1-(trifluoromethyl)cyclopropyl)-1,2,4-oxadiazole-3-yl.

Aspect 32: The compound according to Aspect 1, wherein L_{b} is —(CR^{a}R^{b})ᵤ₁—NR⁷—C(O)—, or —C(O)—NR⁷—(CR^{a}R^{b})ᵤ₁—; wherein u1 is an integral of 0-12. In some embodiment, L_{b} is *—C(O)—NR⁷—(CR^{a}R^{b})ᵤ₁—; wherein u1 is an integral of 1 or 2, R⁷, R^{a} and R^{b} is hydrogen or —C₁₋₈alkyl, and the asterisk * refers to the position attached to Ring A. In some embodiment, L_{b} is *—C(O)—NH—(CR^{a}R^{b})ᵤ₁—; wherein u1 is an integral of 1 or 2, R^{a} and R^{b} is hydrogen or —C₁₋₄alkyl, and the asterisk * refers to the position attached to Ring A. In some embodiment, L_{b} is *—C(O)—NH—CH₂— or *—C(O)—NH—CH(CH₃)—; wherein the asterisk * refers to the position attached to Ring A.

Aspect 33: The compound according to Aspect 1, wherein y is 0 or 1 or 2, and R¹ is halogen or —C₁₋₈alkyl or hydroxyl-C₁₋₈alkyl-, preferably fluoro, chloro, methyl or hydroxymethyl.

Aspect 34: The compound according to Aspect 1, wherein X_{b} is CH and X_{c} is N; or X_{b} is N and X_{c} is CH; or X_{b} is CH and X_{c} is CH.

Aspect 35: The compound according to Aspect 1, wherein X₁ is N and Xₐ is CH; or X₁ is N and Xₐ is N. In some embodiment, t is 0 or 1, and R² is —C₁₋₈alkyl, methoxy or halogen, preferably C₁₋₆alkyl, more preferably methyl. In some embodiment, the

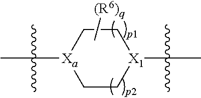

moiety is

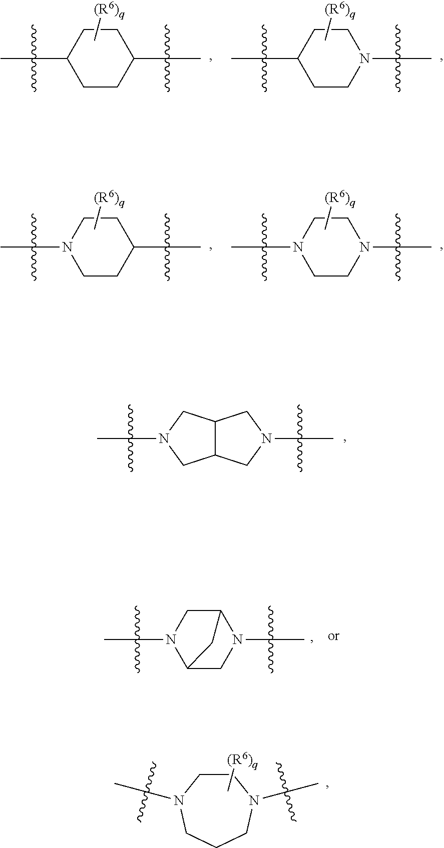

preferably

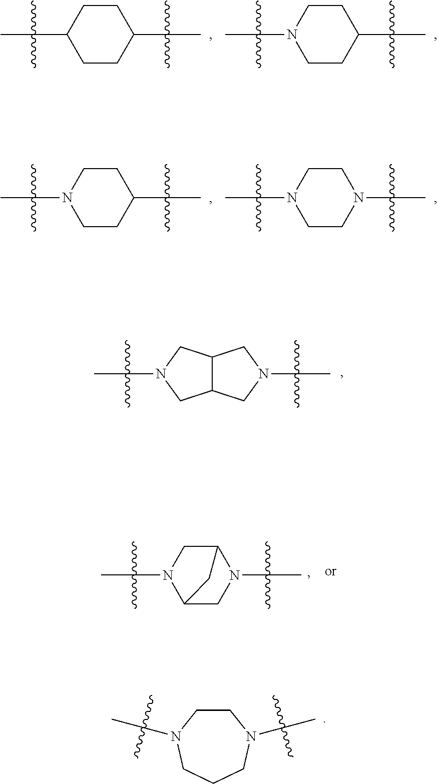

As disclosed herein, the substituent R⁶ can be substituted at any available position in the

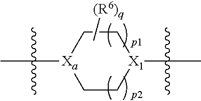

moiety. For example, R⁶ can be substituted at the atom Xa when Xa is CH.

Aspect 36: The compound according to Aspect 1, wherein the compound is selected from

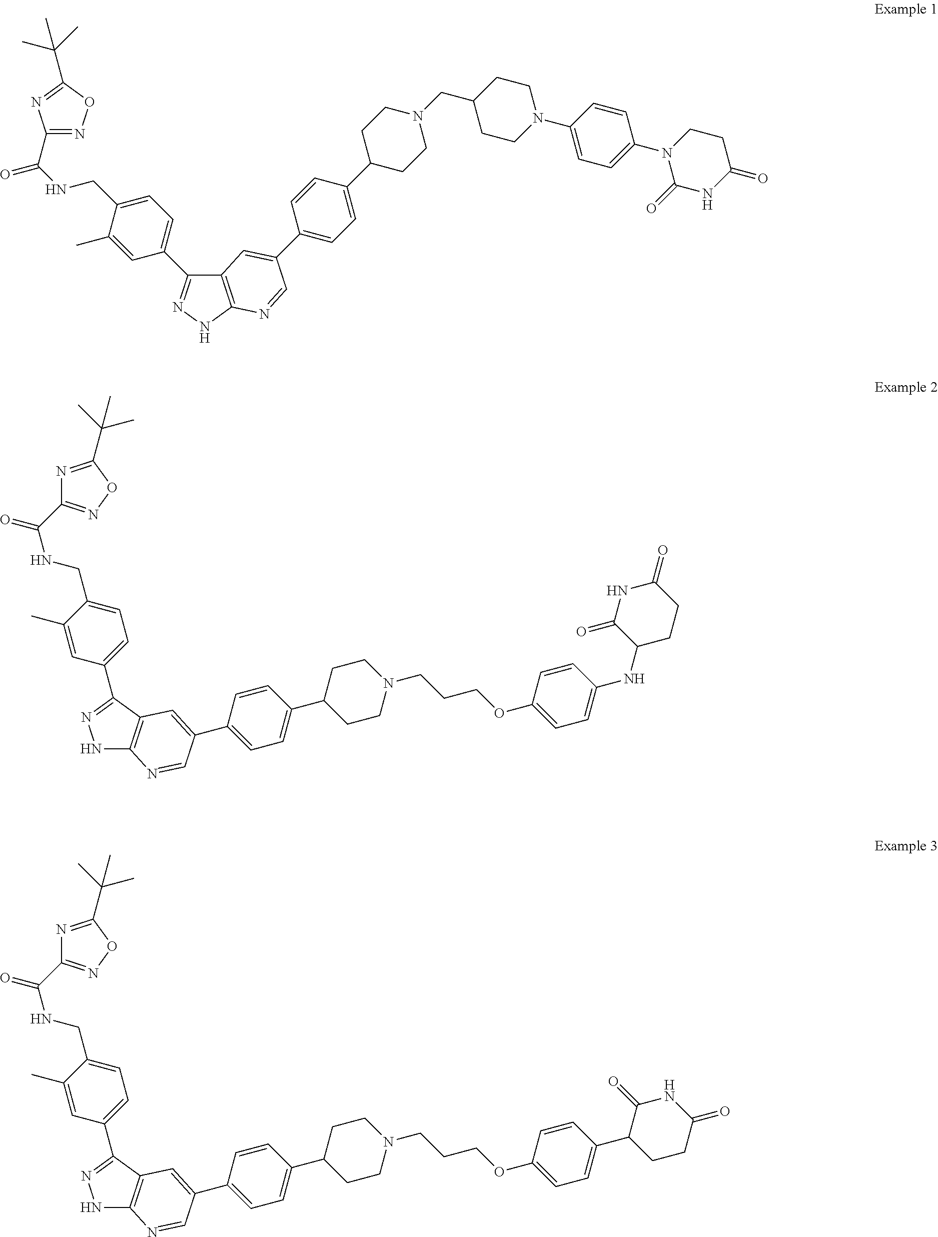 [SMILES: Cc1cc(-c2n[nH]c3ncc(-c4ccc(C5CCN(CC6CCN(c7ccc(N8CCC(=O)NC8=O)cc7)CC6)CC5)cc4)cc23)ccc1CNC(=O)c1noc(C(C)(C)C)n1 ; Cc1cc(-c2n[nH]c3ncc(-c4ccc(C5CCN(CCCOc6ccc(NC7CCC(=O)NC7=O)cc6)CC5)cc4)cc23)ccc1CNC(=O)c1noc(C(C)(C)C)n1 ; Cc1cc(-c2n[nH]c3ncc(-c4ccc(C5CCN(CCCOc6ccc(C7CCC(=O)NC7=O)cc6)CC5)cc4)cc23)ccc1CNC(=O)c1noc(C(C)(C)C)n1]

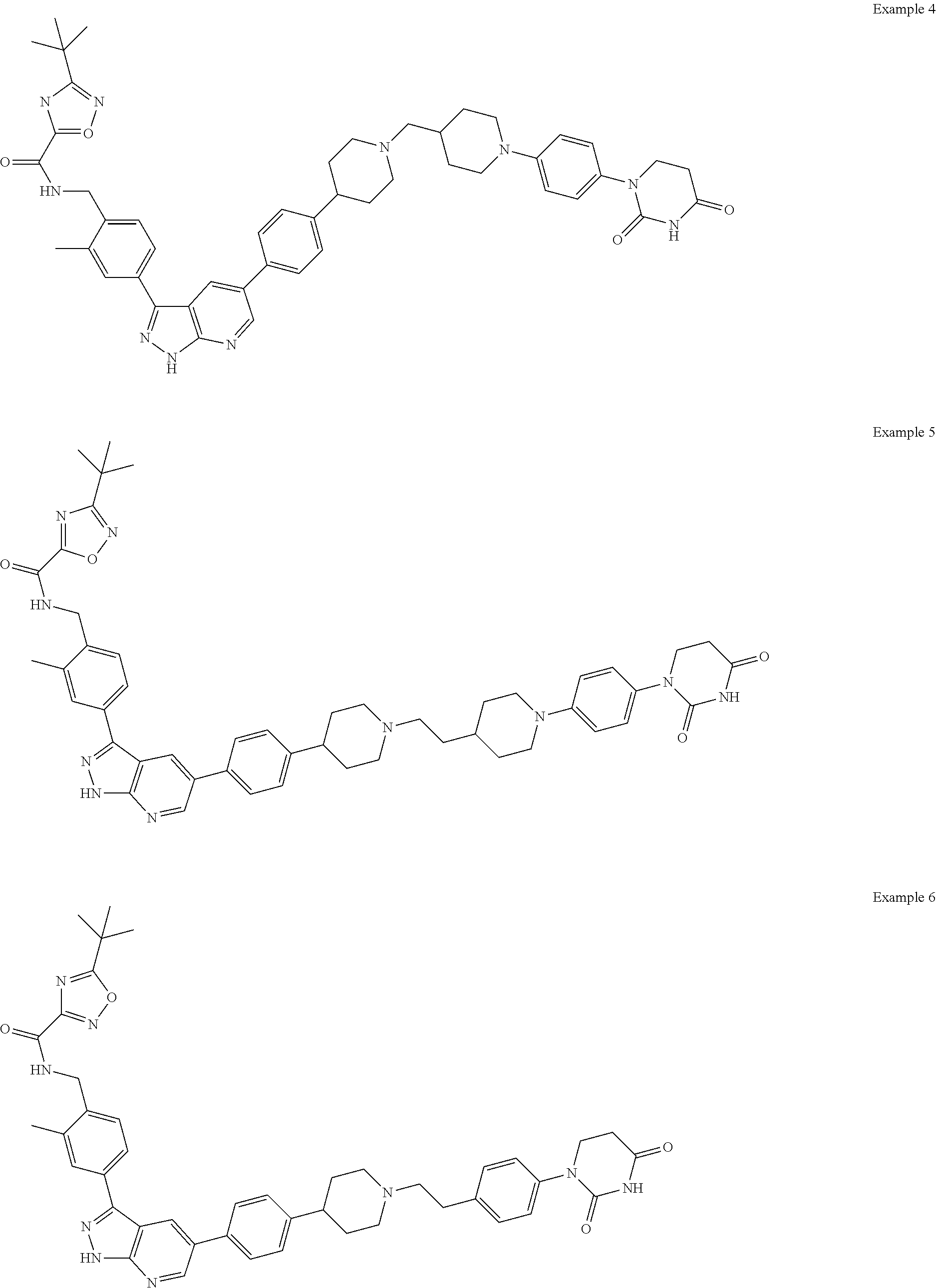

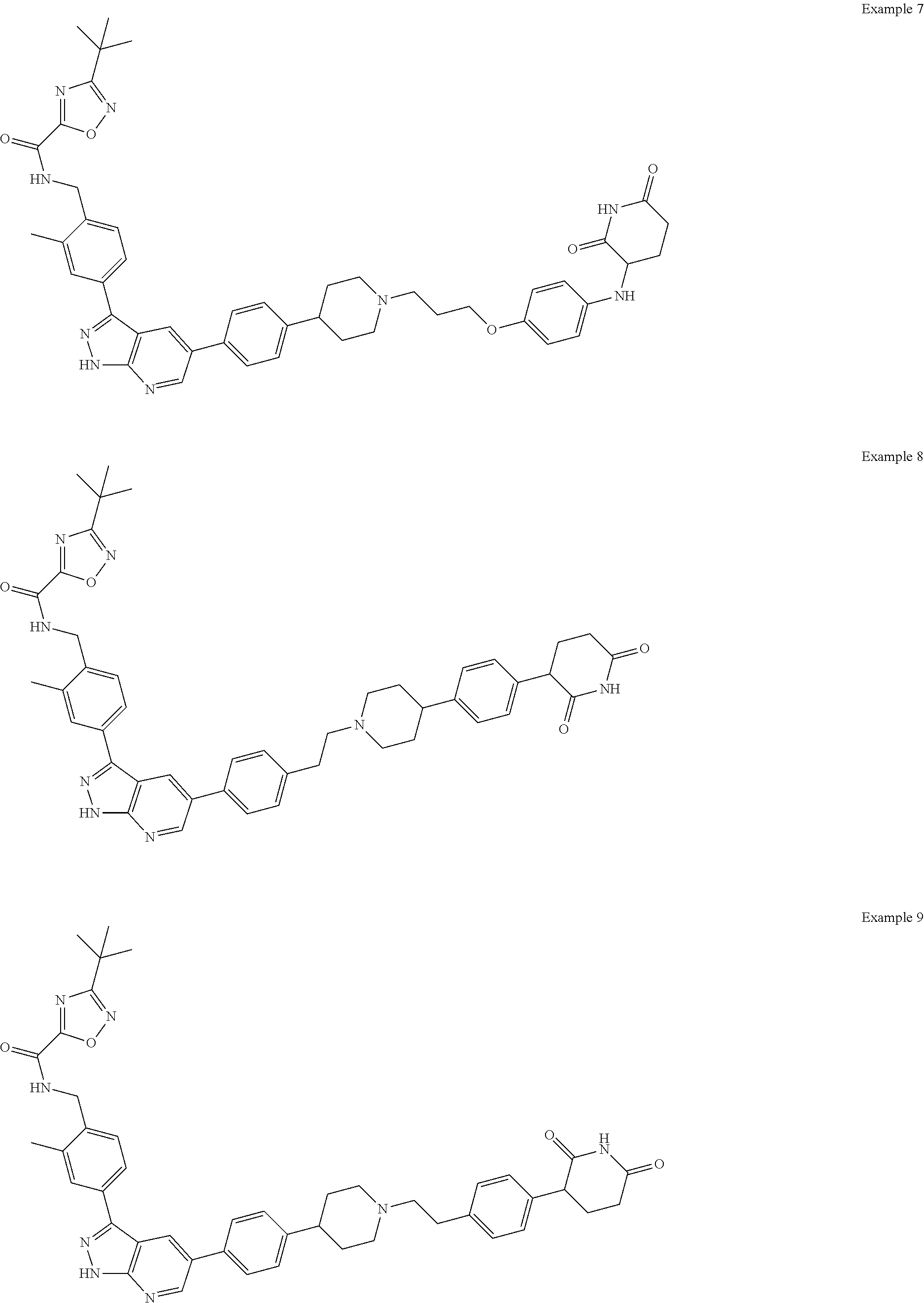 [SMILES: Cc1cc(-c2n[nH]c3ncc(-c4ccc(C5CCN(CCCOc6ccc(NC7CCC(=O)NC7=O)cc6)CC5)cc4)cc23)ccc1CNC(=O)c1nc(C(C)(C)C)no1 ; Cc1cc(-c2n[nH]c3ncc(-c4ccc(CCN5CCC(c6ccc(C7CCC(=O)NC7=O)cc6)CC5)cc4)cc23)ccc1CNC(=O)c1nc(C(C)(C)C)no1 ; Cc1cc(-c2n[nH]c3ncc(-c4ccc(C5CCN(CCc6ccc(C7CCC(=O)NC7=O)cc6)CC5)cc4)cc23)ccc1CNC(=O)c1nc(C(C)(C)C)no1]

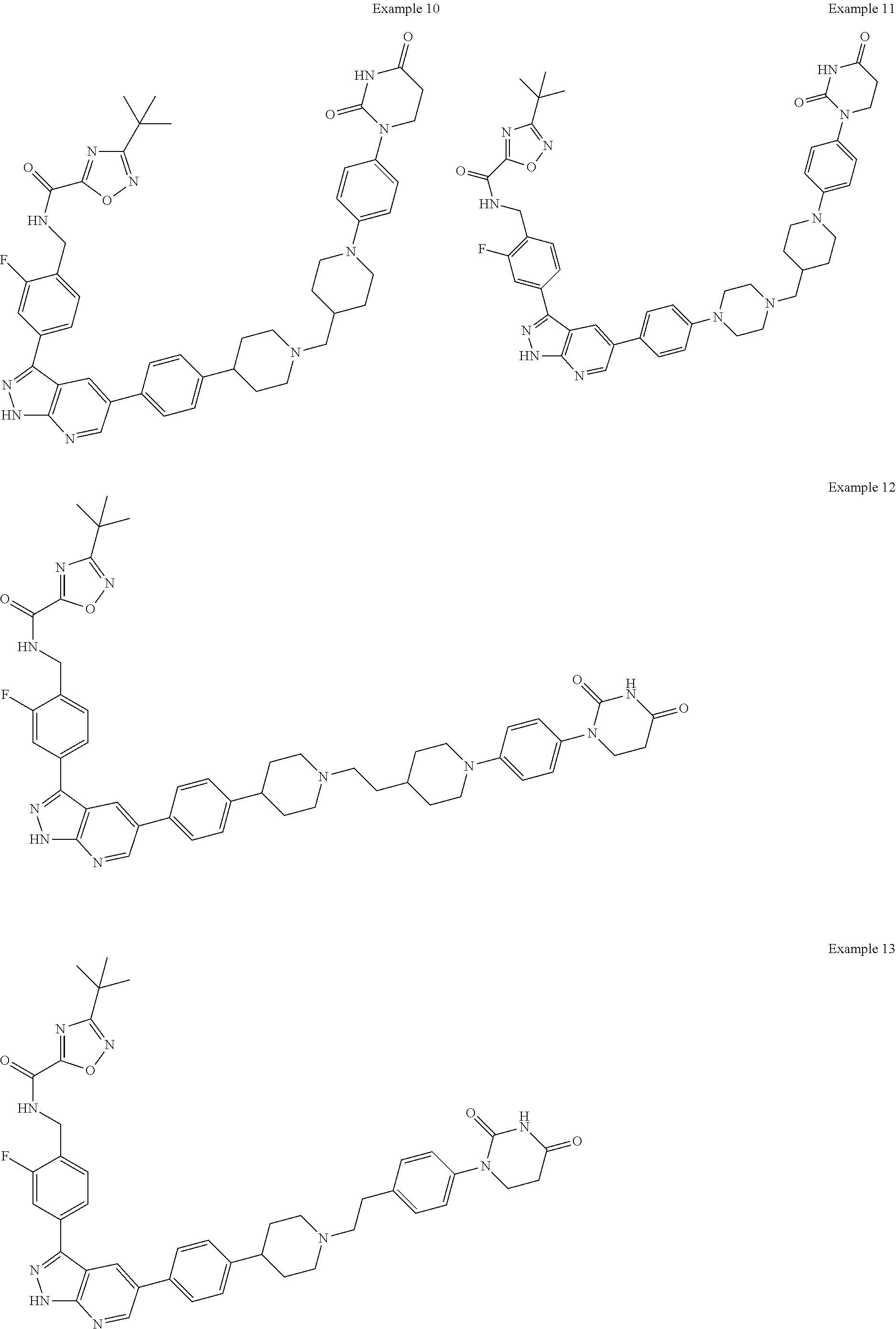 [SMILES: CC(C)(C)c1noc(C(=O)NCc2ccc(-c3n[nH]c4ncc(-c5ccc(C6CCN(CC7CCN(c8ccc(N9CCC(=O)NC9=O)cc8)CC7)CC6)cc5)cc34)cc2F)n1 ; CC(C)(C)c1noc(C(=O)NCc2ccc(-c3n[nH]c4ncc(-c5ccc(N6CCN(CC7CCN(c8ccc(N9CCC(=O)NC9=O)cc8)CC7)CC6)cc5)cc34)cc2F)n1 ; CC(C)(C)c1noc(C(=O)NCc2ccc(-c3n[nH]c4ncc(-c5ccc(C6CCN(CCC7CCN(c8ccc(N9CCC(=O)NC9=O)cc8)CC7)CC6)cc5)cc34)cc2F)n1 ; CC(C)(C)c1noc(C(=O)NCc2ccc(-c3n[nH]c4ncc(-c5ccc(C6CCN(CCc7ccc(N8CCC(=O)NC8=O)cc7)CC6)cc5)cc34)cc2F)n1]

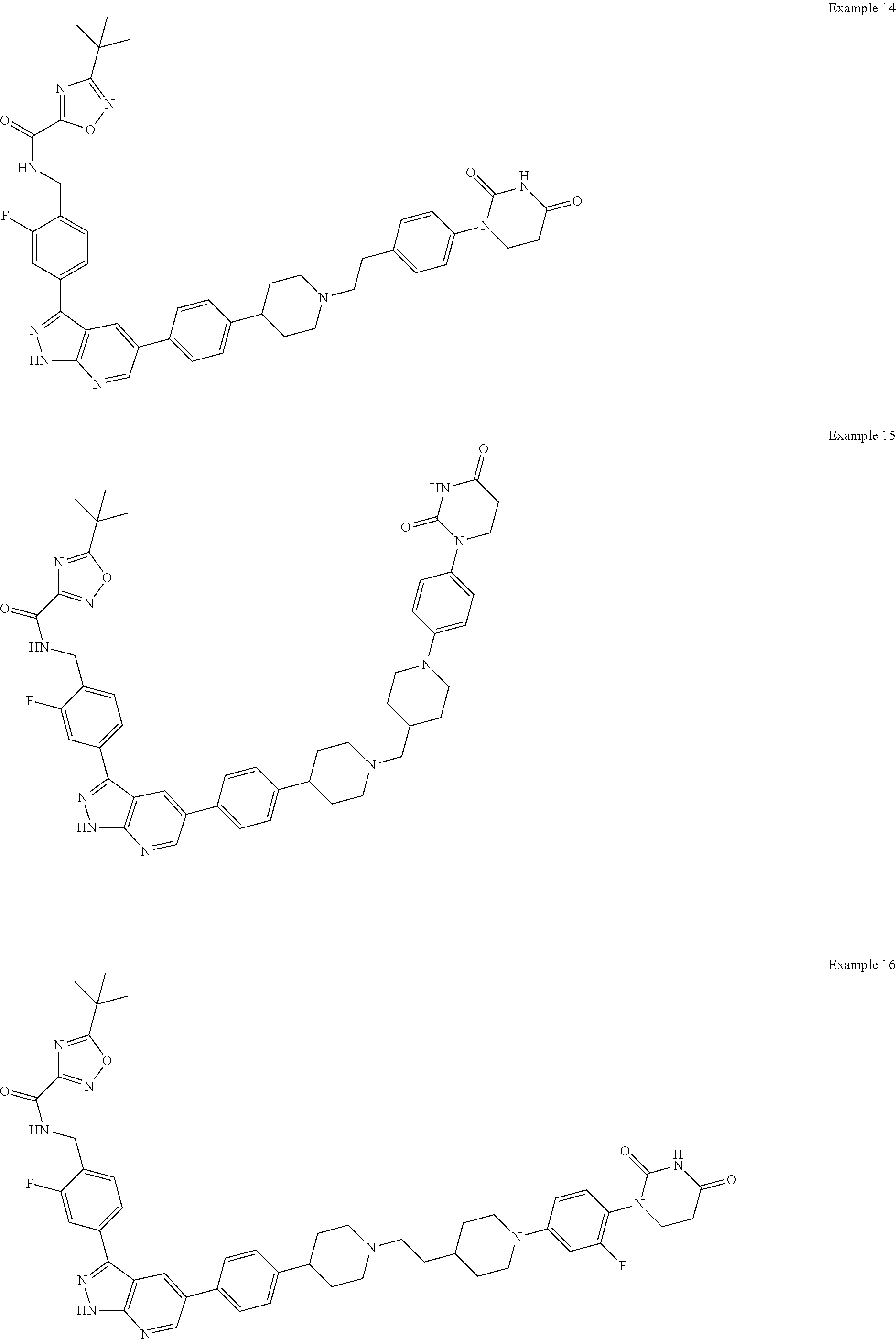 [SMILES: CC(C)(C)c1noc(C(=O)NCc2ccc(-c3n[nH]c4ncc(-c5ccc(C6CCN(CCc7ccc(N8CCC(=O)NC8=O)cc7)CC6)cc5)cc34)cc2F)n1 ; CC(C)(C)c1nc(C(=O)NCc2ccc(-c3n[nH]c4ncc(-c5ccc(C6CCN(CC7CCN(c8ccc(N9CCC(=O)NC9=O)cc8)CC7)CC6)cc5)cc34)cc2F)no1 ; CC(C)(C)c1nc(C(=O)NCc2ccc(-c3n[nH]c4ncc(-c5ccc(C6CCN(CCC7CCN(c8ccc(N9CCC(=O)NC9=O)c(F)c8)CC7)CC6)cc5)cc34)cc2F)no1]

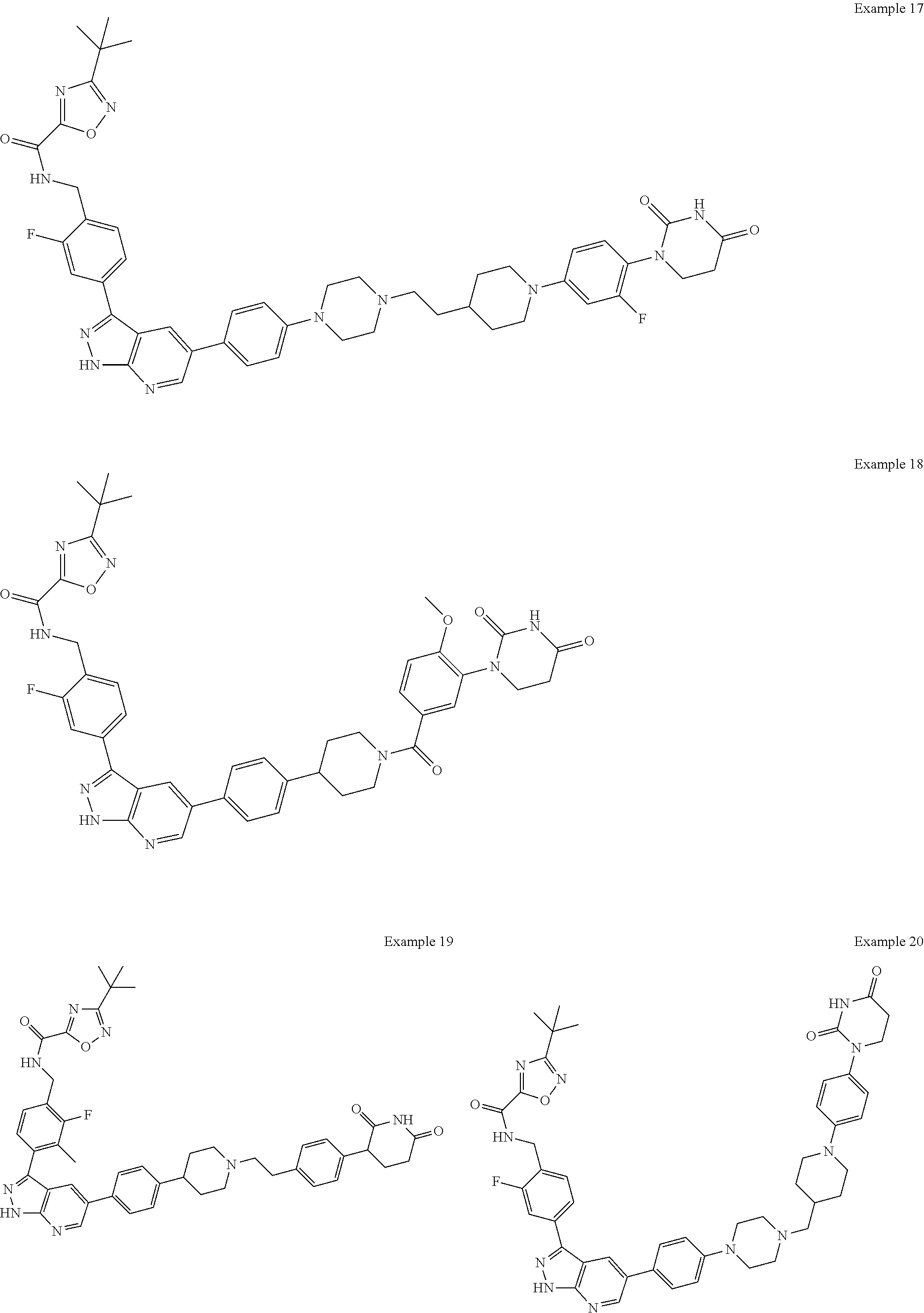

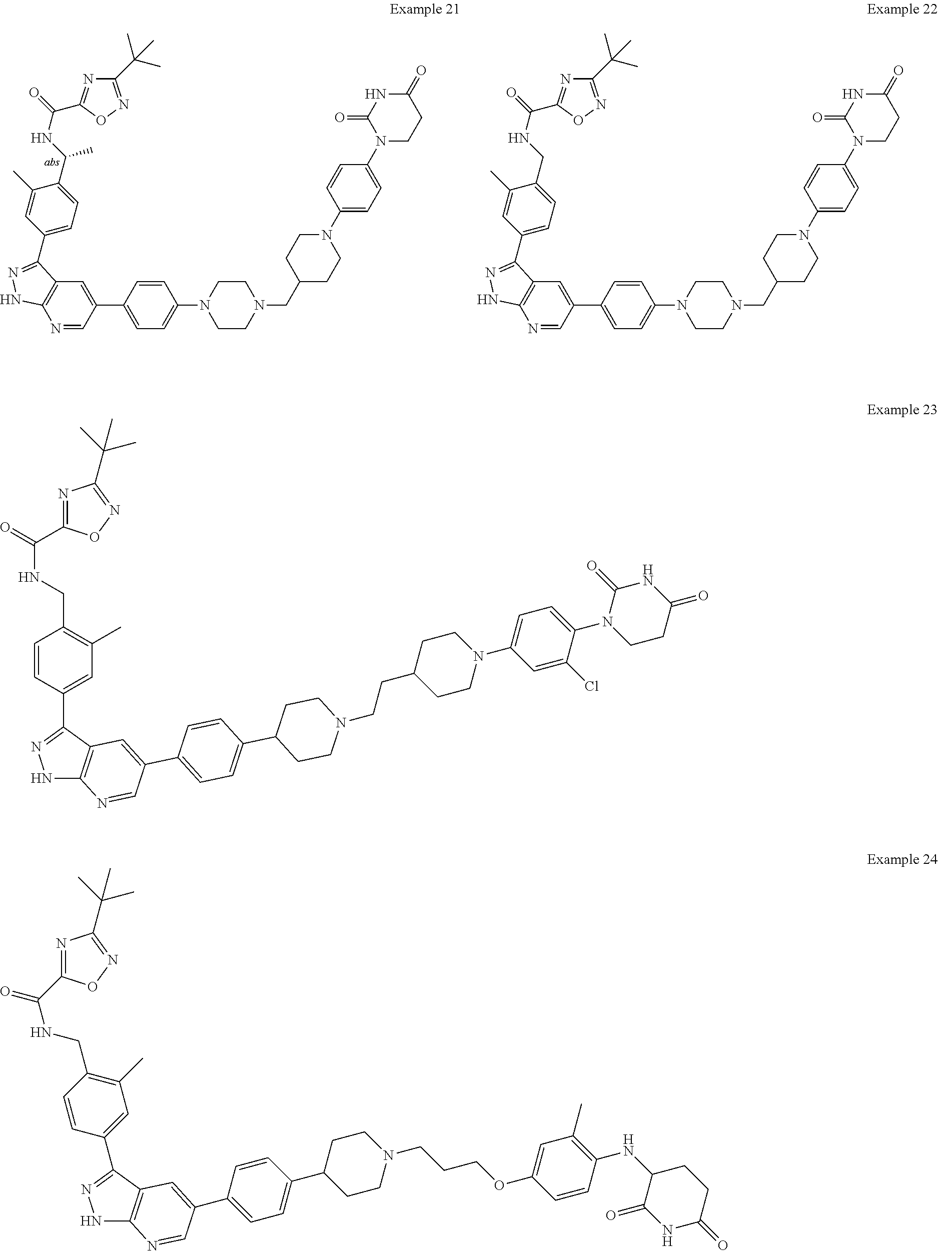 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1[C@@H](C)NC(=O)c1nc(C(C)(C)C)no1 ; Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1nc(C(C)(C)C)no1 ; Cc1cc(-c2n[nH]c3ncc(-c4ccc(C5CCN(CCC6CCN(c7ccc(N8CCC(=O)NC8=O)c(Cl)c7)CC6)CC5)cc4)cc23)ccc1CNC(=O)c1nc(C(C)(C)C)no1 ; Cc1cc(-c2n[nH]c3ncc(-c4ccc(C5CCN(CCCOc6ccc(NC7CCC(=O)NC7=O)c(C)c6)CC5)cc4)cc23)ccc1CNC(=O)c1nc(C(C)(C)C)no1]

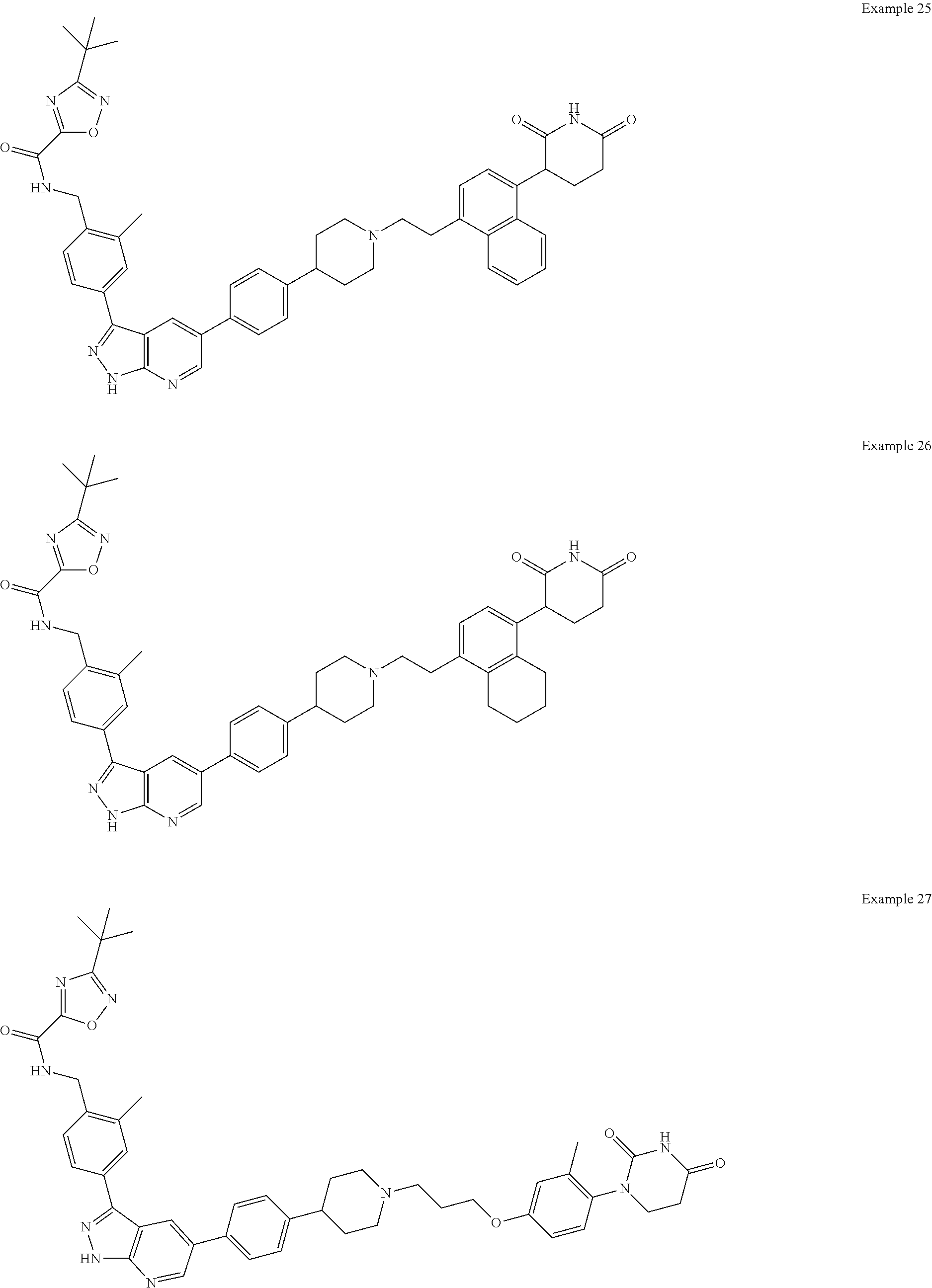 [SMILES: Cc1cc(-c2n[nH]c3ncc(-c4ccc(C5CCN(CCc6ccc(C7CCC(=O)NC7=O)c7ccccc67)CC5)cc4)cc23)ccc1CNC(=O)c1nc(C(C)(C)C)no1 ; Cc1cc(-c2n[nH]c3ncc(-c4ccc(C5CCN(CCc6ccc(C7CCC(=O)NC7=O)c7c6CCCC7)CC5)cc4)cc23)ccc1CNC(=O)c1nc(C(C)(C)C)no1 ; Cc1cc(-c2n[nH]c3ncc(-c4ccc(C5CCN(CCCOc6ccc(N7CCC(=O)NC7=O)c(C)c6)CC5)cc4)cc23)ccc1CNC(=O)c1nc(C(C)(C)C)no1]

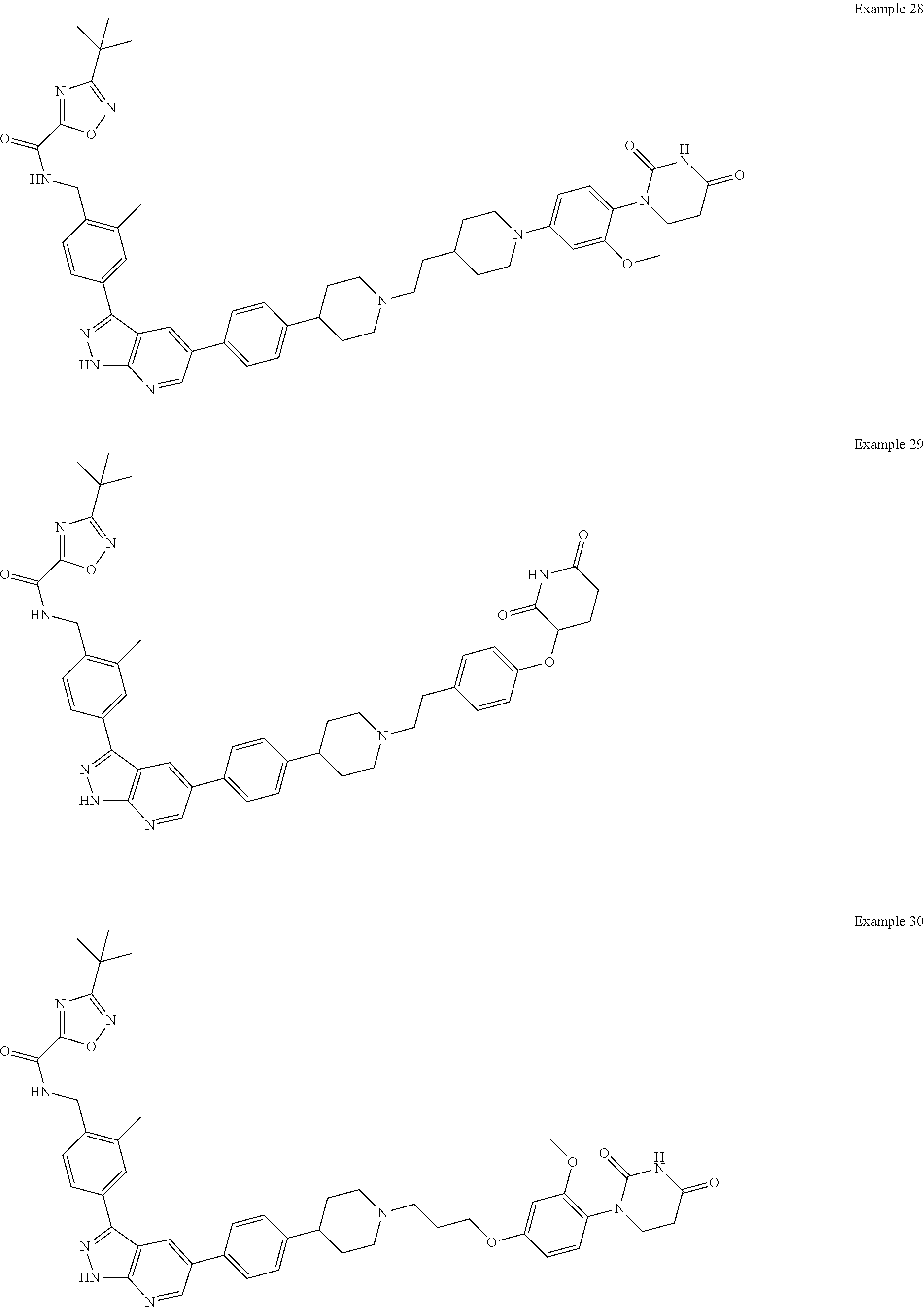 [SMILES: COc1cc(N2CCC(CCN3CCC(c4ccc(-c5cnc6[nH]nc(-c7ccc(CNC(=O)c8nc(C(C)(C)C)no8)c(C)c7)c6c5)cc4)CC3)CC2)ccc1N1CCC(=O)NC1=O ; Cc1cc(-c2n[nH]c3ncc(-c4ccc(C5CCN(CCc6ccc(OC7CCC(=O)NC7=O)cc6)CC5)cc4)cc23)ccc1CNC(=O)c1nc(C(C)(C)C)no1 ; COc1cc(OCCCN2CCC(c3ccc(-c4cnc5[nH]nc(-c6ccc(CNC(=O)c7nc(C(C)(C)C)no7)c(C)c6)c5c4)cc3)CC2)ccc1N1CCC(=O)NC1=O]

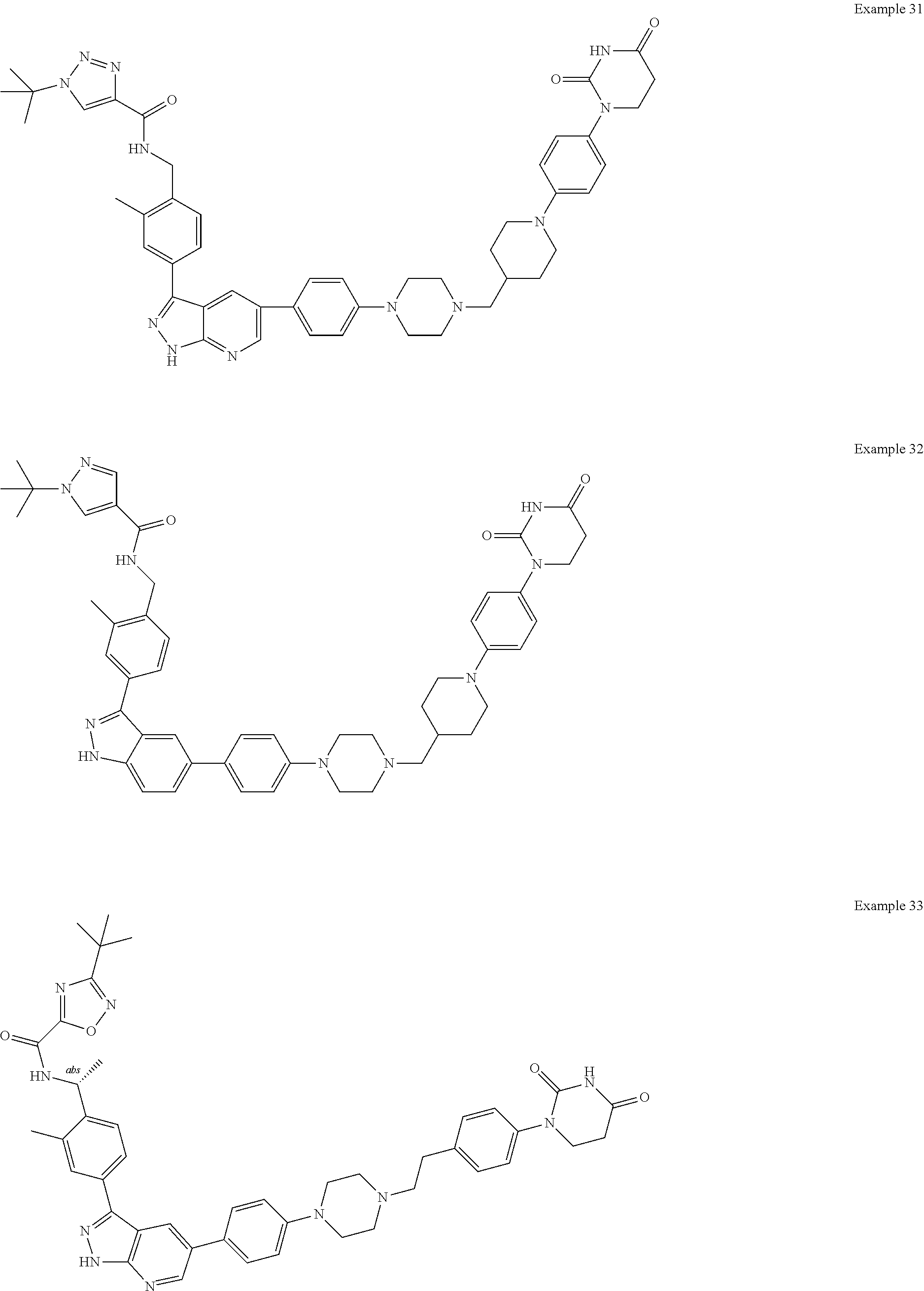 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1cn(C(C)(C)C)nn1 ; Cc1cc(-c2n[nH]c3ccc(-c4ccc(N5CCN(CC6CCN(c7ccc(N8CCC(=O)NC8=O)cc7)CC6)CC5)cc4)cc23)ccc1CNC(=O)c1cnn(C(C)(C)C)c1 ; Cc1cc(-c2n[nH]c3ncc(-c4ccc(N5CCN(CCc6ccc(N7CCC(=O)NC7=O)cc6)CC5)cc4)cc23)ccc1[C@@H](C)NC(=O)c1nc(C(C)(C)C)no1]

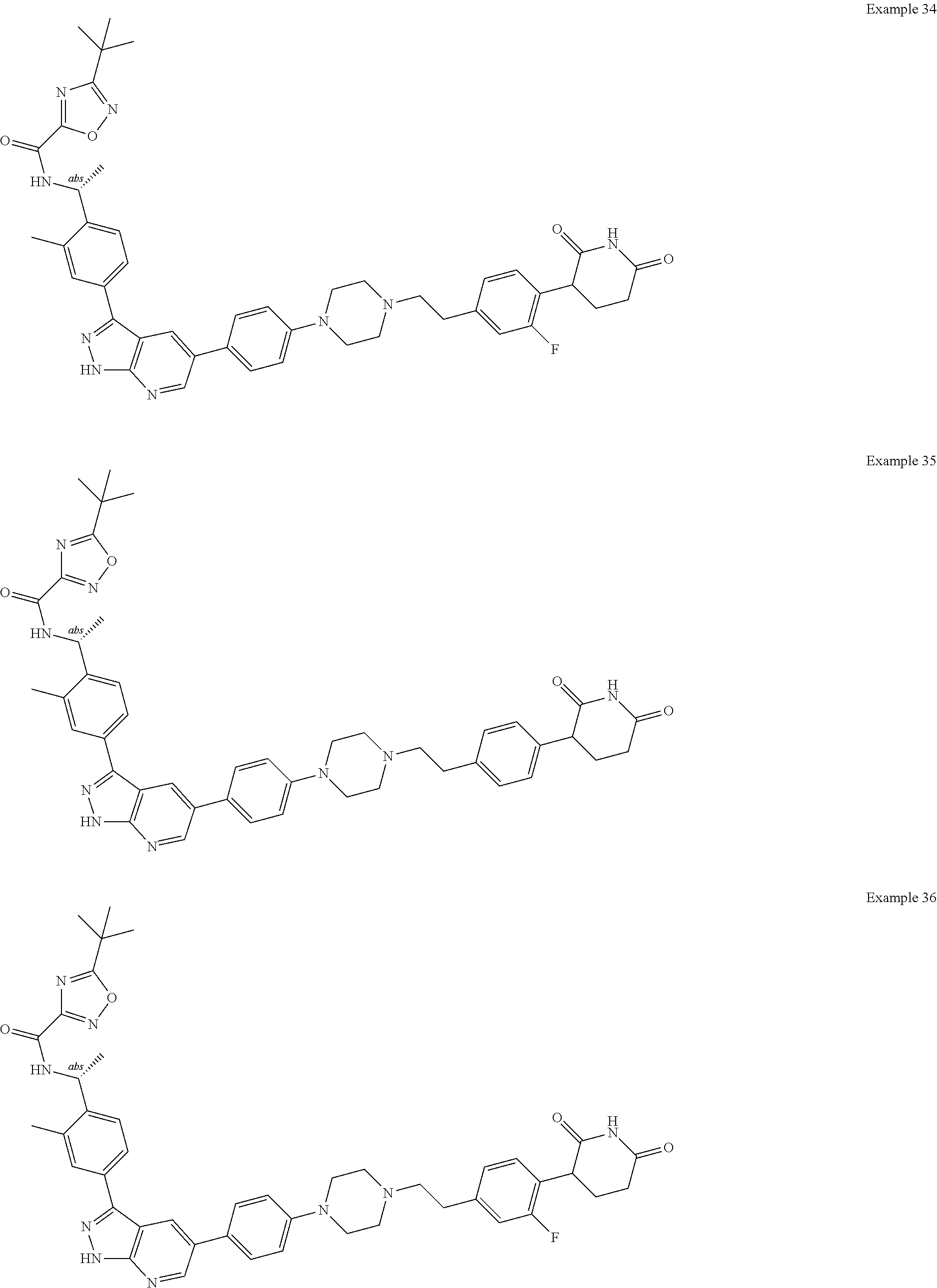 [SMILES: Cc1cc(-c2n[nH]c3ncc(-c4ccc(N5CCN(CCc6ccc(C7CCC(=O)NC7=O)c(F)c6)CC5)cc4)cc23)ccc1[C@@H](C)NC(=O)c1nc(C(C)(C)C)no1 ; Cc1cc(-c2n[nH]c3ncc(-c4ccc(N5CCN(CCc6ccc(C7CCC(=O)NC7=O)cc6)CC5)cc4)cc23)ccc1[C@@H](C)NC(=O)c1noc(C(C)(C)C)n1 ; Cc1cc(-c2n[nH]c3ncc(-c4ccc(N5CCN(CCc6ccc(C7CCC(=O)NC7=O)c(F)c6)CC5)cc4)cc23)ccc1[C@@H](C)NC(=O)c1noc(C(C)(C)C)n1]

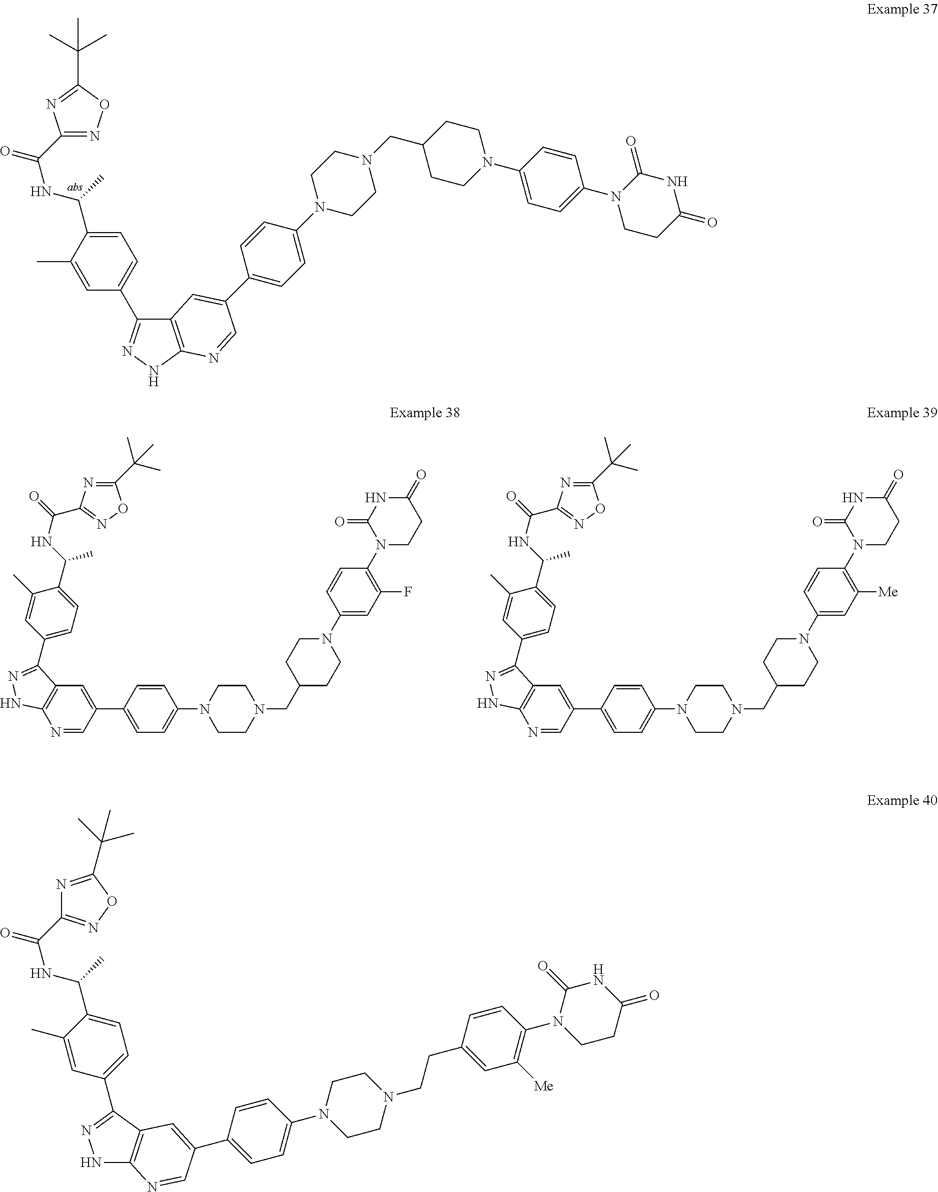 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1[C@@H](C)NC(=O)c1noc(C(C)(C)C)n1 ; Cc1cc(-c2n[nH]c3ncc(-c4ccc(N5CCN(CC6CCN(c7ccc(N8CCC(=O)NC8=O)c(F)c7)CC6)CC5)cc4)cc23)ccc1[C@@H](C)NC(=O)c1noc(C(C)(C)C)n1 ; Cc1cc(-c2n[nH]c3ncc(-c4ccc(N5CCN(CC6CCN(c7ccc(N8CCC(=O)NC8=O)c(C)c7)CC6)CC5)cc4)cc23)ccc1[C@@H](C)NC(=O)c1noc(C(C)(C)C)n1 ; Cc1cc(-c2n[nH]c3ncc(-c4ccc(N5CCN(CCc6ccc(N7CCC(=O)NC7=O)c(C)c6)CC5)cc4)cc23)ccc1[C@@H](C)NC(=O)c1noc(C(C)(C)C)n1]

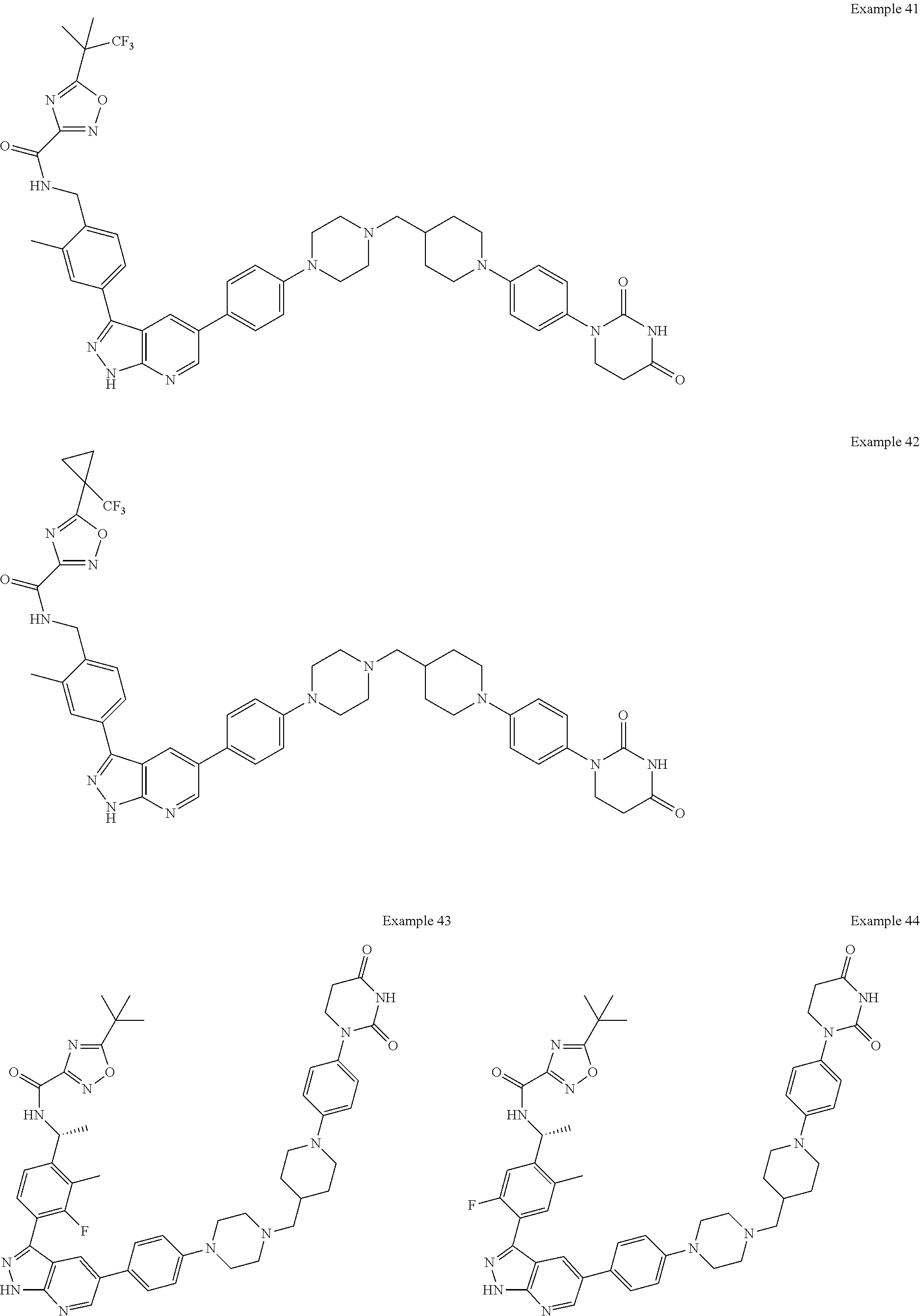 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1noc(C(C)(C)C(F)(F)F)n1 ; Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1noc(C2(C(F)(F)F)CC2)n1 ; Cc1c([C@@H](C)NC(=O)c2noc(C(C)(C)C)n2)ccc(-c2n[nH]c3ncc(-c4ccc(N5CCN(CC6CCN(c7ccc(N8CCC(=O)NC8=O)cc7)CC6)CC5)cc4)cc23)c1F ; Cc1cc(-c2n[nH]c3ncc(-c4ccc(N5CCN(CC6CCN(c7ccc(N8CCC(=O)NC8=O)cc7)CC6)CC5)cc4)cc23)c(F)cc1[C@@H](C)NC(=O)c1noc(C(C)(C)C)n1]

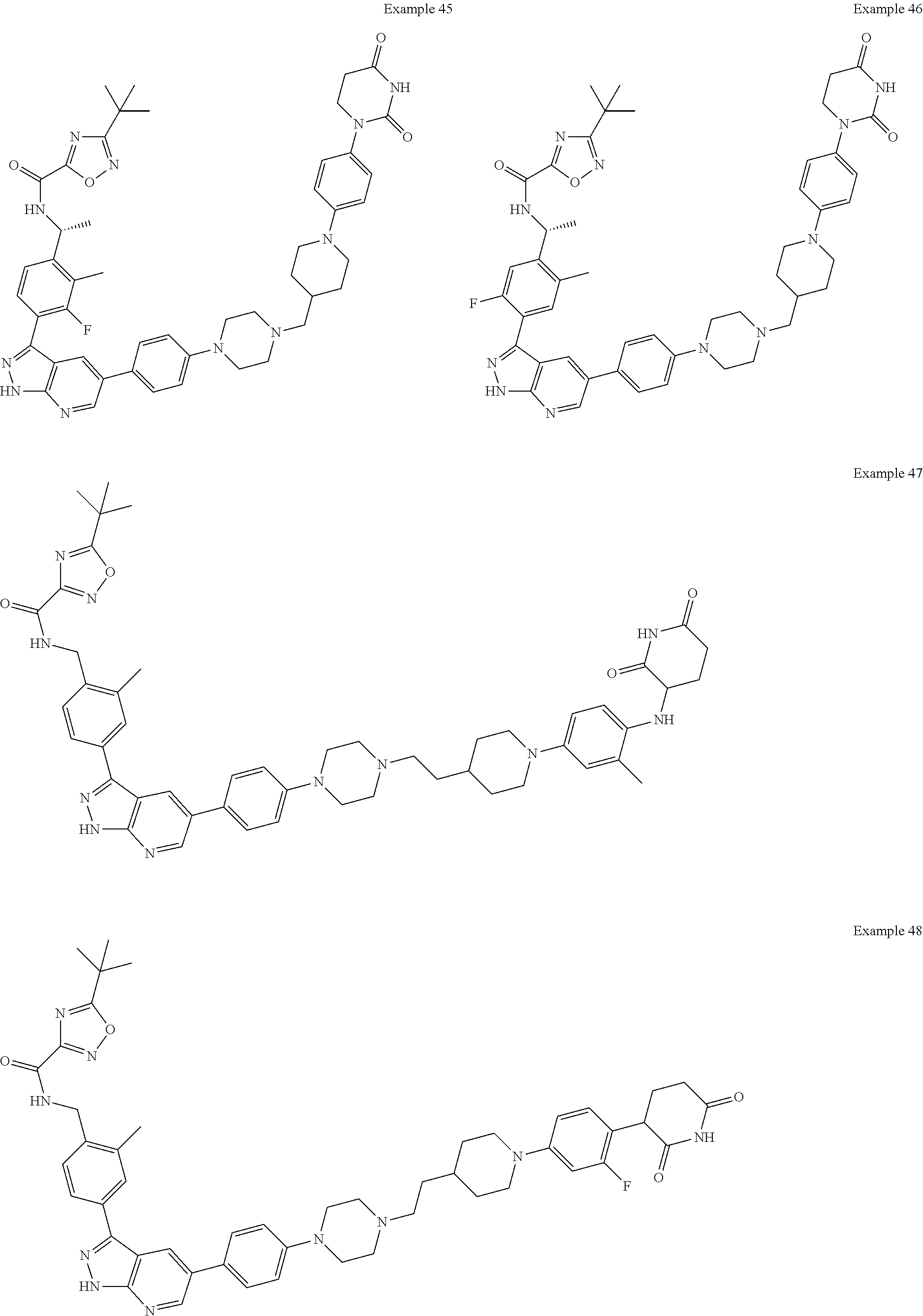 [SMILES: Cc1c([C@@H](C)NC(=O)c2nc(C(C)(C)C)no2)ccc(-c2n[nH]c3ncc(-c4ccc(N5CCN(CC6CCN(c7ccc(N8CCC(=O)NC8=O)cc7)CC6)CC5)cc4)cc23)c1F ; Cc1cc(-c2n[nH]c3ncc(-c4ccc(N5CCN(CC6CCN(c7ccc(N8CCC(=O)NC8=O)cc7)CC6)CC5)cc4)cc23)c(F)cc1[C@@H](C)NC(=O)c1nc(C(C)(C)C)no1 ; Cc1cc(-c2n[nH]c3ncc(-c4ccc(N5CCN(CCC6CCN(c7ccc(NC8CCC(=O)NC8=O)c(C)c7)CC6)CC5)cc4)cc23)ccc1CNC(=O)c1noc(C(C)(C)C)n1 ; Cc1cc(-c2n[nH]c3ncc(-c4ccc(N5CCN(CCC6CCN(c7ccc(C8CCC(=O)NC8=O)c(F)c7)CC6)CC5)cc4)cc23)ccc1CNC(=O)c1noc(C(C)(C)C)n1]

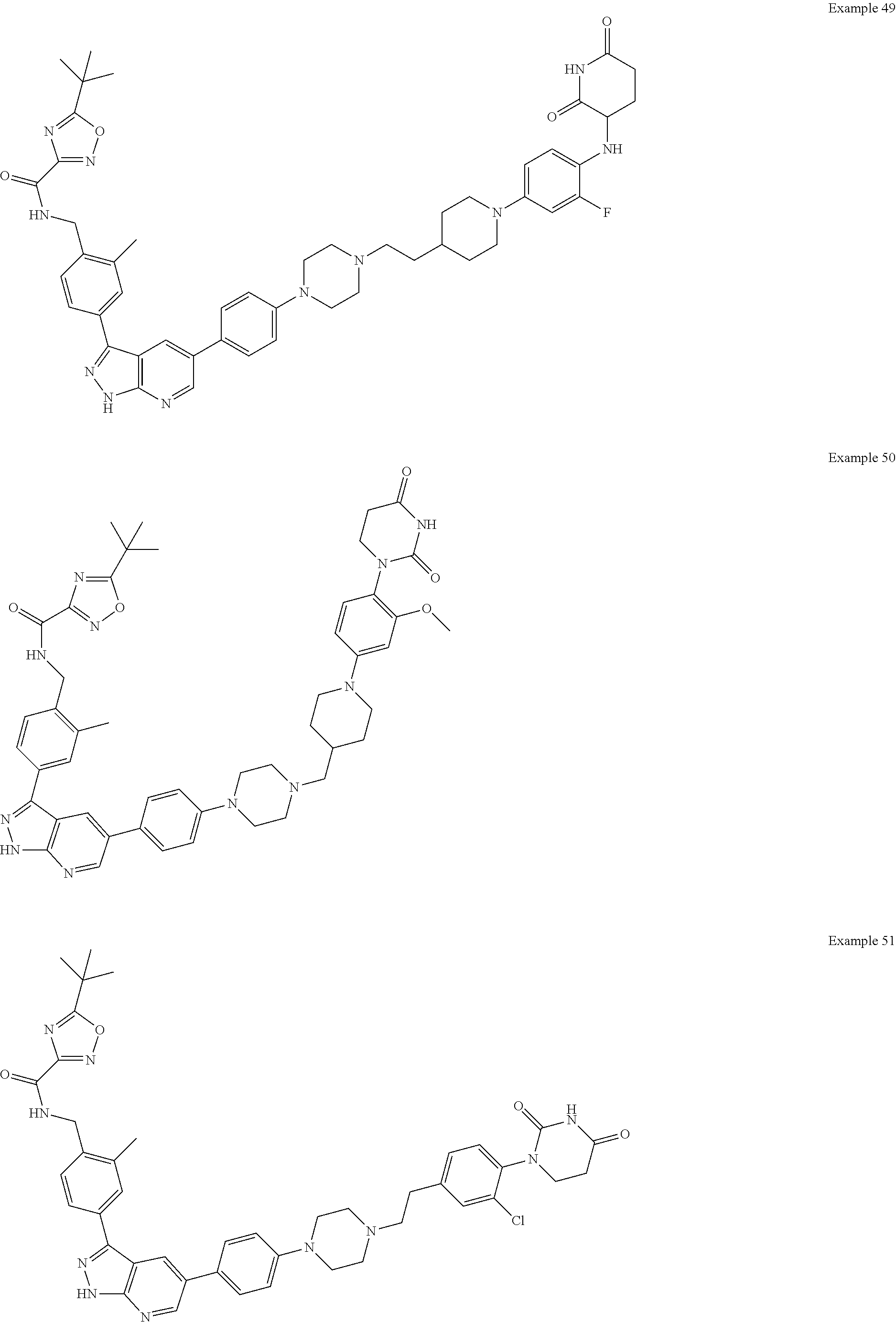 [SMILES: Cc1cc(-c2n[nH]c3ncc(-c4ccc(N5CCN(CCC6CCN(c7ccc(NC8CCC(=O)NC8=O)c(F)c7)CC6)CC5)cc4)cc23)ccc1CNC(=O)c1noc(C(C)(C)C)n1 ; COc1cc(N2CCC(CN3CCN(c4ccc(-c5cnc6[nH]nc(-c7ccc(CNC(=O)c8noc(C(C)(C)C)n8)c(C)c7)c6c5)cc4)CC3)CC2)ccc1N1CCC(=O)NC1=O ; Cc1cc(-c2n[nH]c3ncc(-c4ccc(N5CCN(CCc6ccc(N7CCC(=O)NC7=O)c(Cl)c6)CC5)cc4)cc23)ccc1CNC(=O)c1noc(C(C)(C)C)n1]

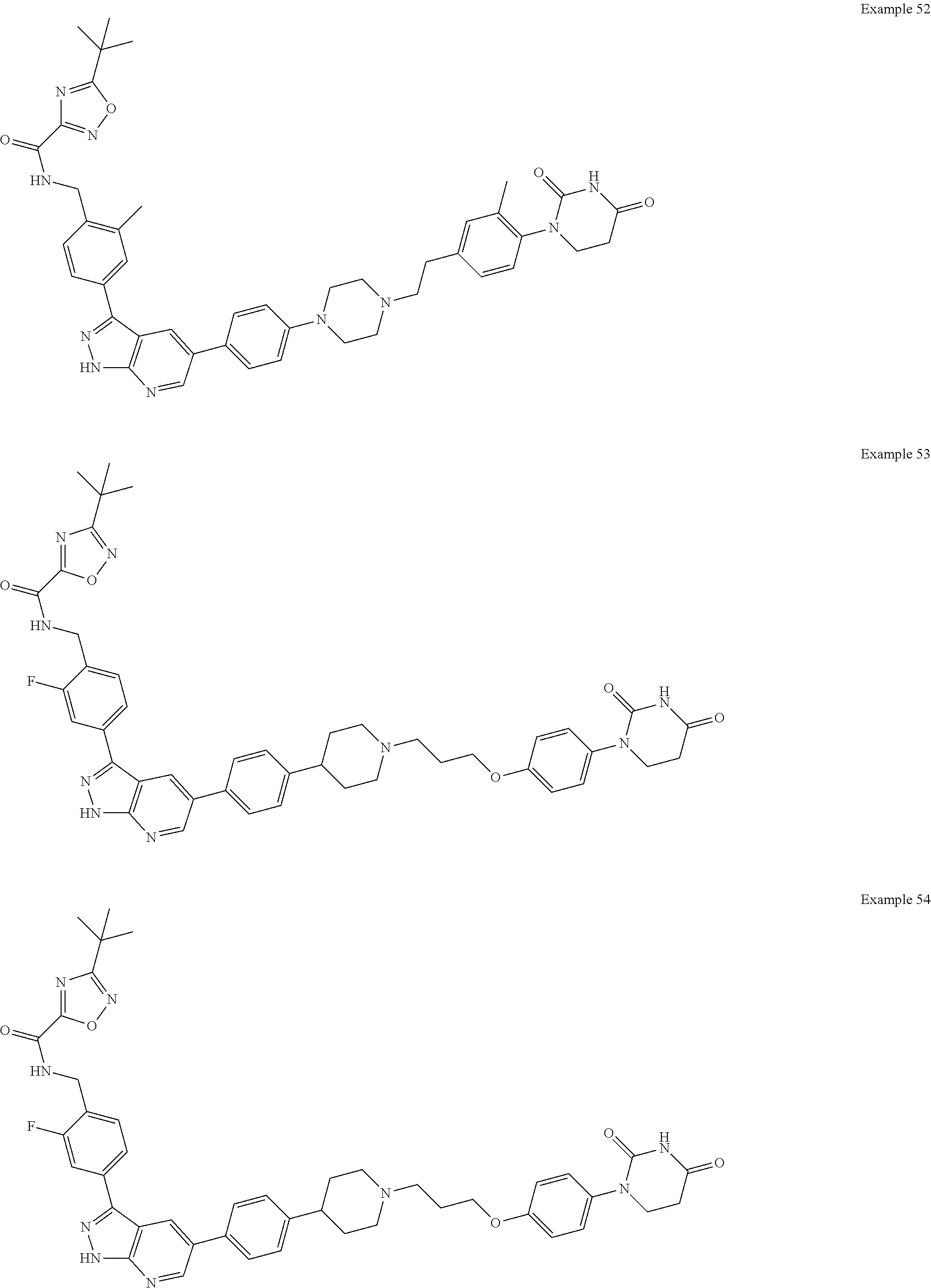 [SMILES: Cc1cc(-c2n[nH]c3ncc(-c4ccc(N5CCN(CCc6ccc(N7CCC(=O)NC7=O)c(C)c6)CC5)cc4)cc23)ccc1CNC(=O)c1noc(C(C)(C)C)n1 ; CC(C)(C)c1noc(C(=O)NCc2ccc(-c3n[nH]c4ncc(-c5ccc(C6CCN(CCCOc7ccc(N8CCC(=O)NC8=O)cc7)CC6)cc5)cc34)cc2F)n1 ; CC(C)(C)c1noc(C(=O)NCc2ccc(-c3n[nH]c4ncc(-c5ccc(C6CCN(CCCOc7ccc(N8CCC(=O)NC8=O)cc7)CC6)cc5)cc34)cc2F)n1]

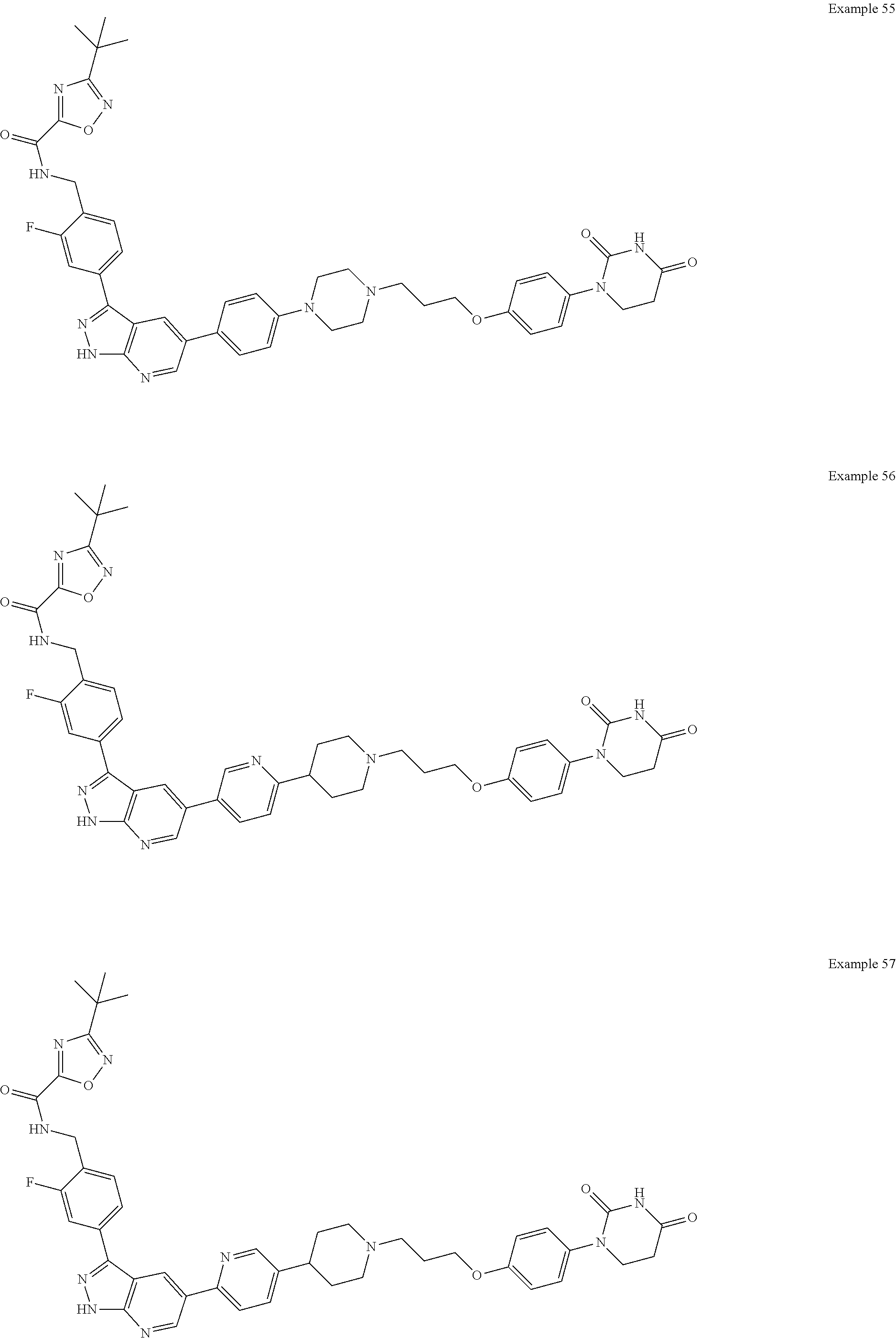 [SMILES: CC(C)(C)c1noc(C(=O)NCc2ccc(-c3n[nH]c4ncc(-c5ccc(N6CCN(CCCOc7ccc(N8CCC(=O)NC8=O)cc7)CC6)cc5)cc34)cc2F)n1 ; CC(C)(C)c1noc(C(=O)NCc2ccc(-c3n[nH]c4ncc(-c5ccc(C6CCN(CCCOc7ccc(N8CCC(=O)NC8=O)cc7)CC6)nc5)cc34)cc2F)n1 ; CC(C)(C)c1noc(C(=O)NCc2ccc(-c3n[nH]c4ncc(-c5ccc(C6CCN(CCCOc7ccc(N8CCC(=O)NC8=O)cc7)CC6)cn5)cc34)cc2F)n1]

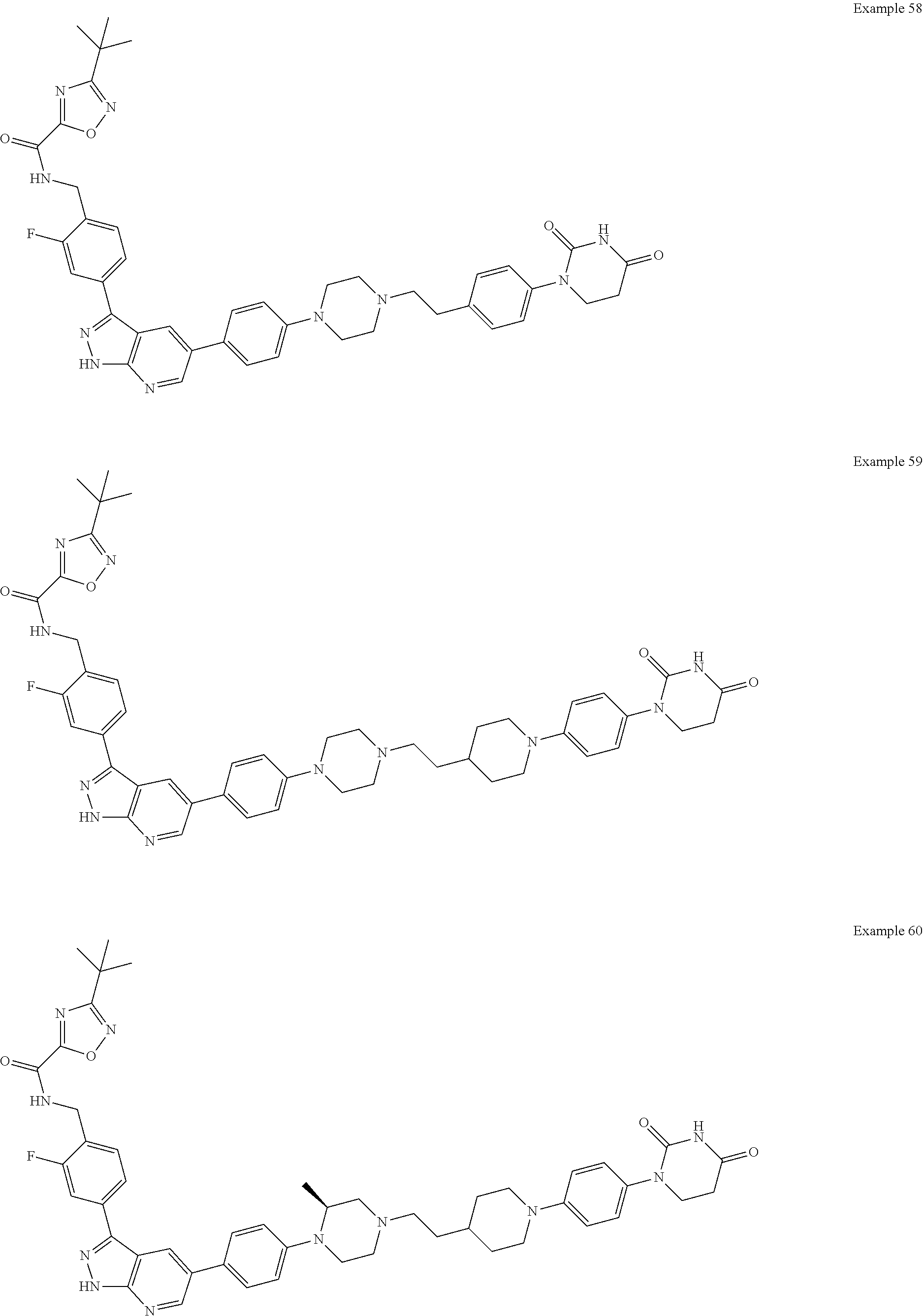 [SMILES: CC(C)(C)c1noc(C(=O)NCc2ccc(-c3n[nH]c4ncc(-c5ccc(N6CCN(CCc7ccc(N8CCC(=O)NC8=O)cc7)CC6)cc5)cc34)cc2F)n1 ; CC(C)(C)c1noc(C(=O)NCc2ccc(-c3n[nH]c4ncc(-c5ccc(N6CCN(CCC7CCN(c8ccc(N9CCC(=O)NC9=O)cc8)CC7)CC6)cc5)cc34)cc2F)n1 ; C[C@H]1CN(CCC2CCN(c3ccc(N4CCC(=O)NC4=O)cc3)CC2)CCN1c1ccc(-c2cnc3[nH]nc(-c4ccc(CNC(=O)c5nc(C(C)(C)C)no5)c(F)c4)c3c2)cc1]

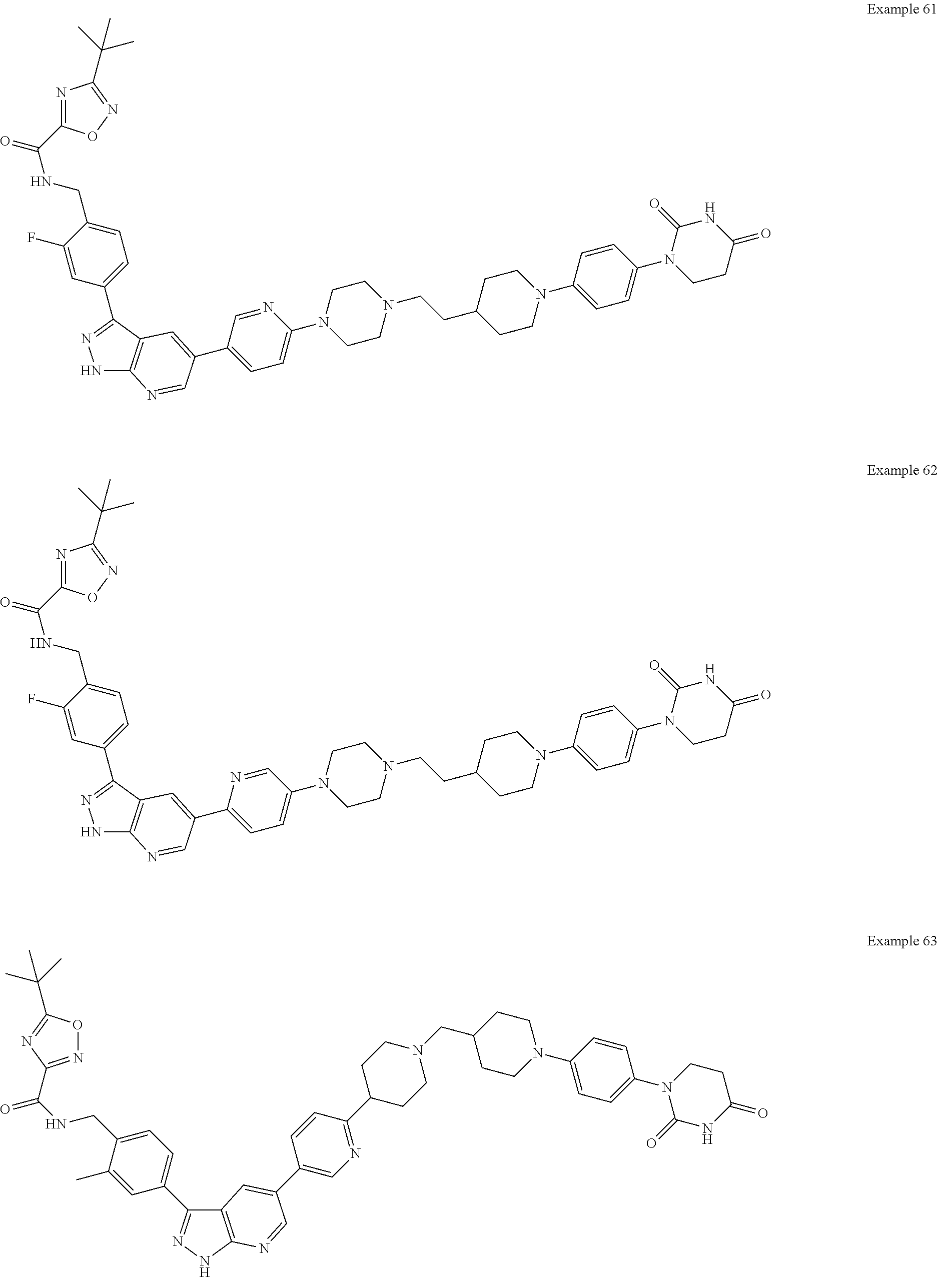 [SMILES: CC(C)(C)c1noc(C(=O)NCc2ccc(-c3n[nH]c4ncc(-c5ccc(N6CCN(CCC7CCN(c8ccc(N9CCC(=O)NC9=O)cc8)CC7)CC6)nc5)cc34)cc2F)n1 ; CC(C)(C)c1noc(C(=O)NCc2ccc(-c3n[nH]c4ncc(-c5ccc(N6CCN(CCC7CCN(c8ccc(N9CCC(=O)NC9=O)cc8)CC7)CC6)cn5)cc34)cc2F)n1 ; Cc1cc(-c2n[nH]c3ncc(-c4ccc(C5CCN(CC6CCN(c7ccc(N8CCC(=O)NC8=O)cc7)CC6)CC5)nc4)cc23)ccc1CNC(=O)c1noc(C(C)(C)C)n1]

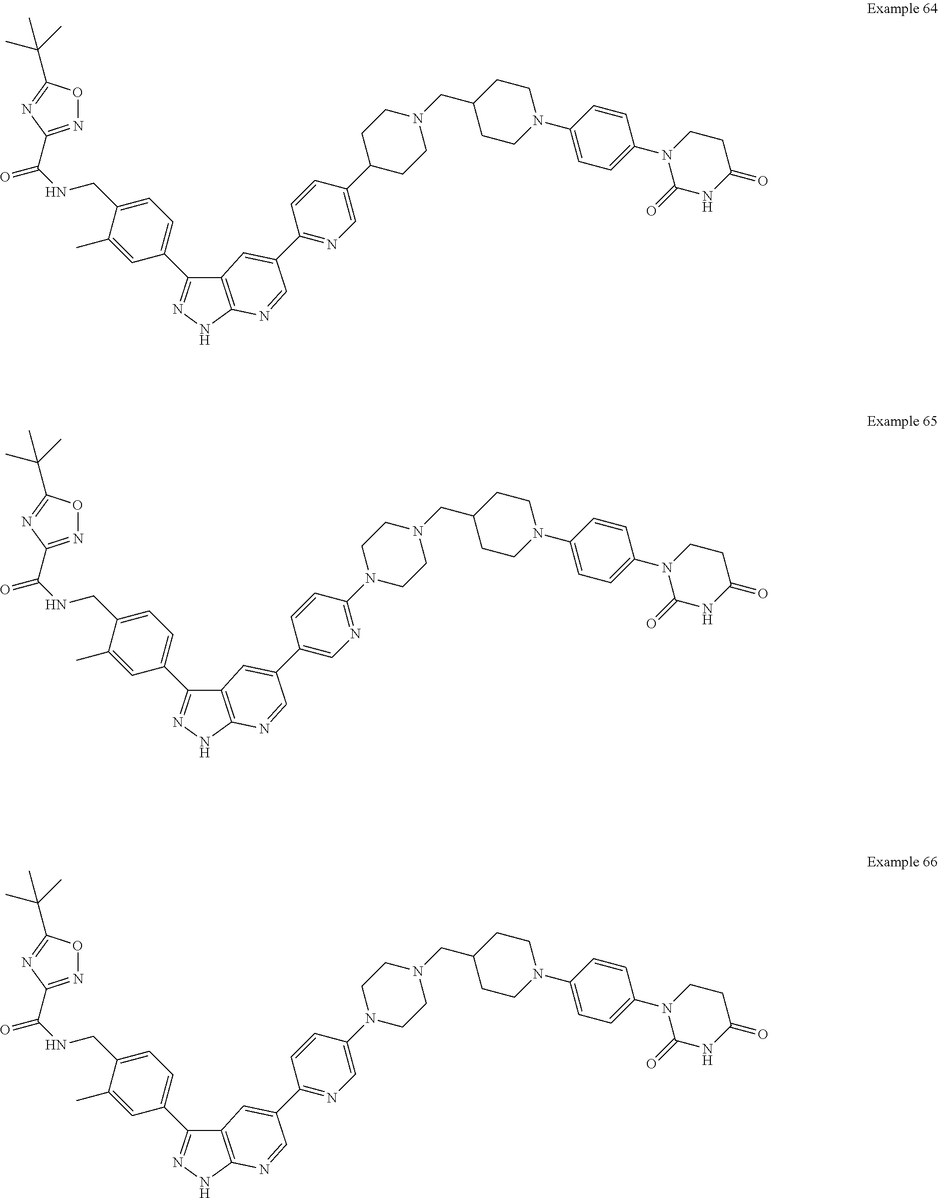 [SMILES: Cc1cc(-c2n[nH]c3ncc(-c4ccc(C5CCN(CC6CCN(c7ccc(N8CCC(=O)NC8=O)cc7)CC6)CC5)cn4)cc23)ccc1CNC(=O)c1noc(C(C)(C)C)n1 ; Cc1cc(-c2n[nH]c3ncc(-c4ccc(N5CCN(CC6CCN(c7ccc(N8CCC(=O)NC8=O)cc7)CC6)CC5)nc4)cc23)ccc1CNC(=O)c1noc(C(C)(C)C)n1 ; Cc1cc(-c2n[nH]c3ncc(-c4ccc(N5CCN(CC6CCN(c7ccc(N8CCC(=O)NC8=O)cc7)CC6)CC5)cn4)cc23)ccc1CNC(=O)c1noc(C(C)(C)C)n1]

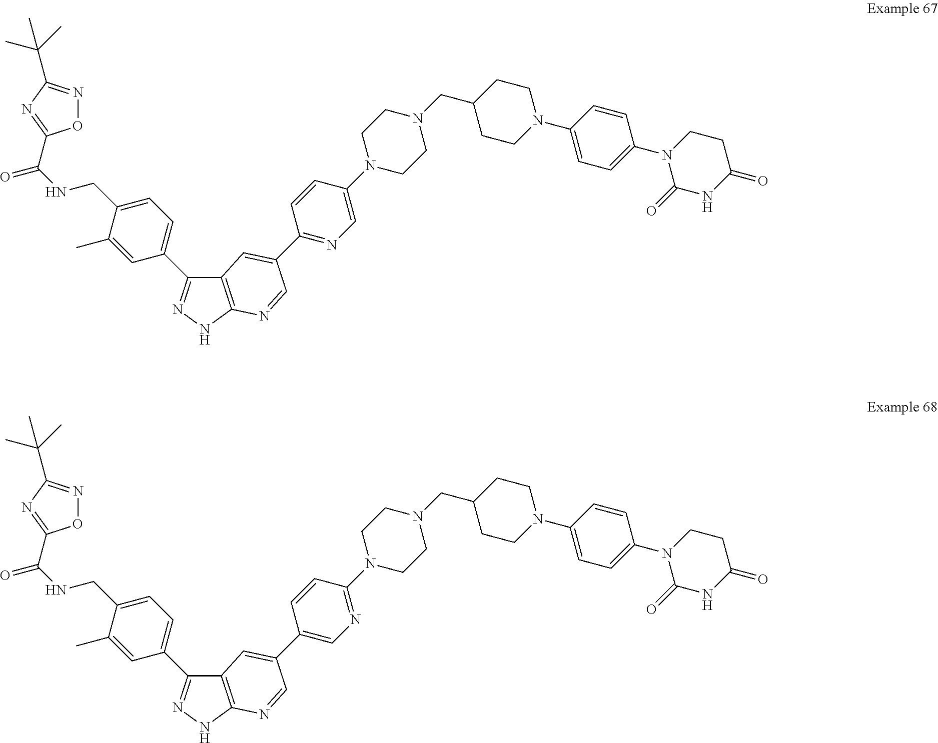 [SMILES: Cc1cc(-c2n[nH]c3ncc(-c4ccc(N5CCN(CC6CCN(c7ccc(N8CCC(=O)NC8=O)cc7)CC6)CC5)cn4)cc23)ccc1CNC(=O)c1nc(C(C)(C)C)no1 ; Cc1cc(-c2n[nH]c3ncc(-c4ccc(N5CCN(CC6CCN(c7ccc(N8CCC(=O)NC8=O)cc7)CC6)CC5)nc4)cc23)ccc1CNC(=O)c1nc(C(C)(C)C)no1]

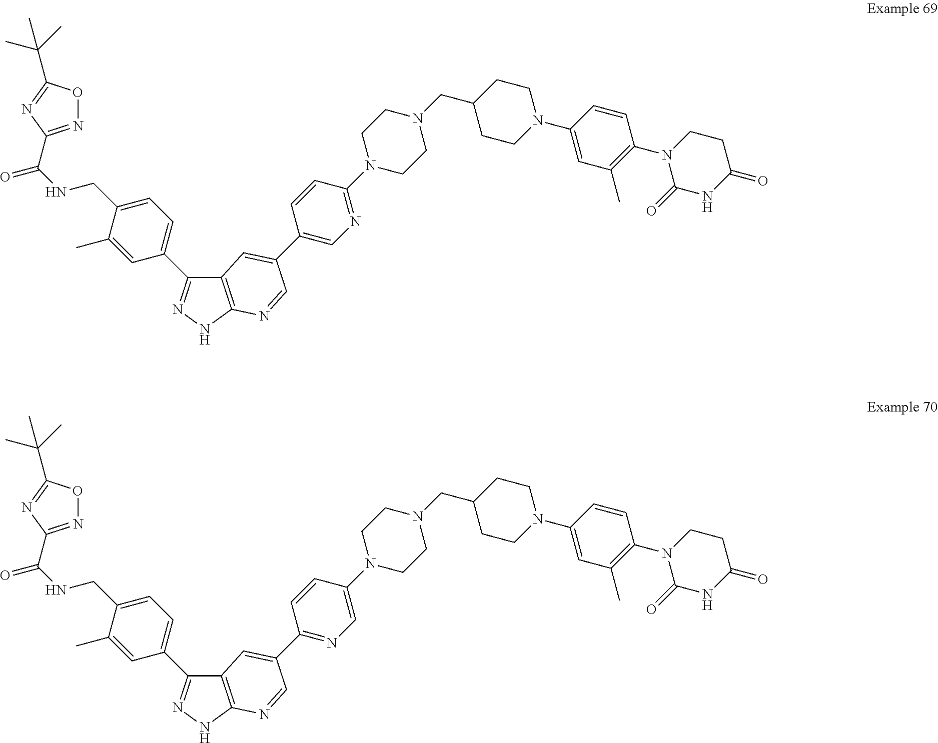 [SMILES: Cc1cc(-c2n[nH]c3ncc(-c4ccc(N5CCN(CC6CCN(c7ccc(N8CCC(=O)NC8=O)c(C)c7)CC6)CC5)nc4)cc23)ccc1CNC(=O)c1noc(C(C)(C)C)n1 ; Cc1cc(-c2n[nH]c3ncc(-c4ccc(N5CCN(CC6CCN(c7ccc(N8CCC(=O)NC8=O)c(C)c7)CC6)CC5)cn4)cc23)ccc1CNC(=O)c1noc(C(C)(C)C)n1]

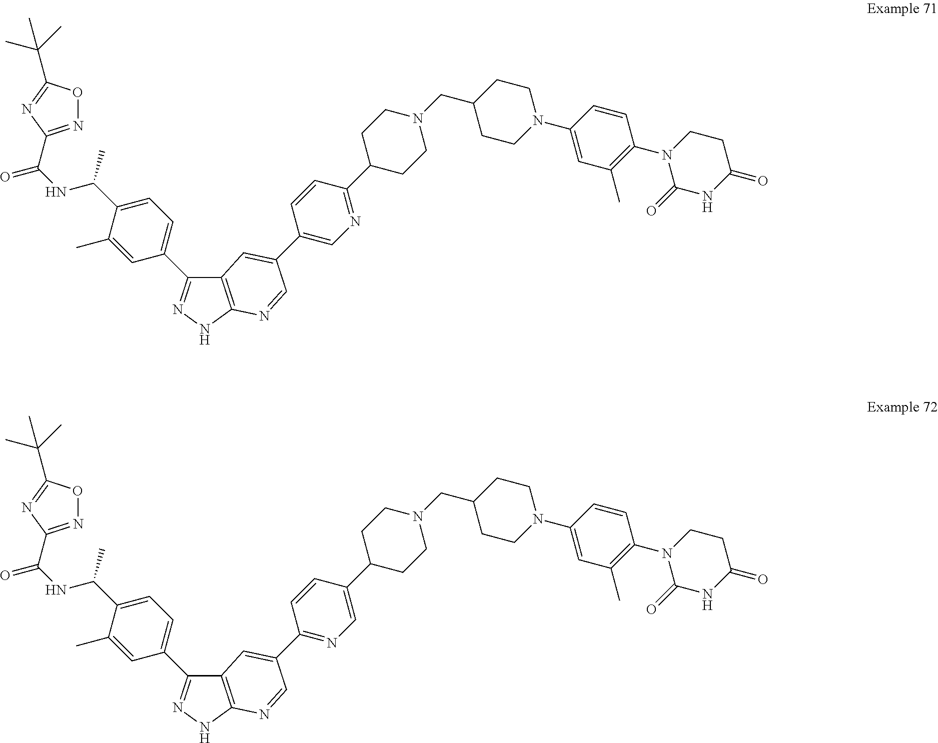 [SMILES: Cc1cc(-c2n[nH]c3ncc(-c4ccc(C5CCN(CC6CCN(c7ccc(N8CCC(=O)NC8=O)c(C)c7)CC6)CC5)nc4)cc23)ccc1[C@@H](C)NC(=O)c1noc(C(C)(C)C)n1 ; Cc1cc(-c2n[nH]c3ncc(-c4ccc(C5CCN(CC6CCN(c7ccc(N8CCC(=O)NC8=O)c(C)c7)CC6)CC5)cn4)cc23)ccc1[C@@H](C)NC(=O)c1noc(C(C)(C)C)n1]

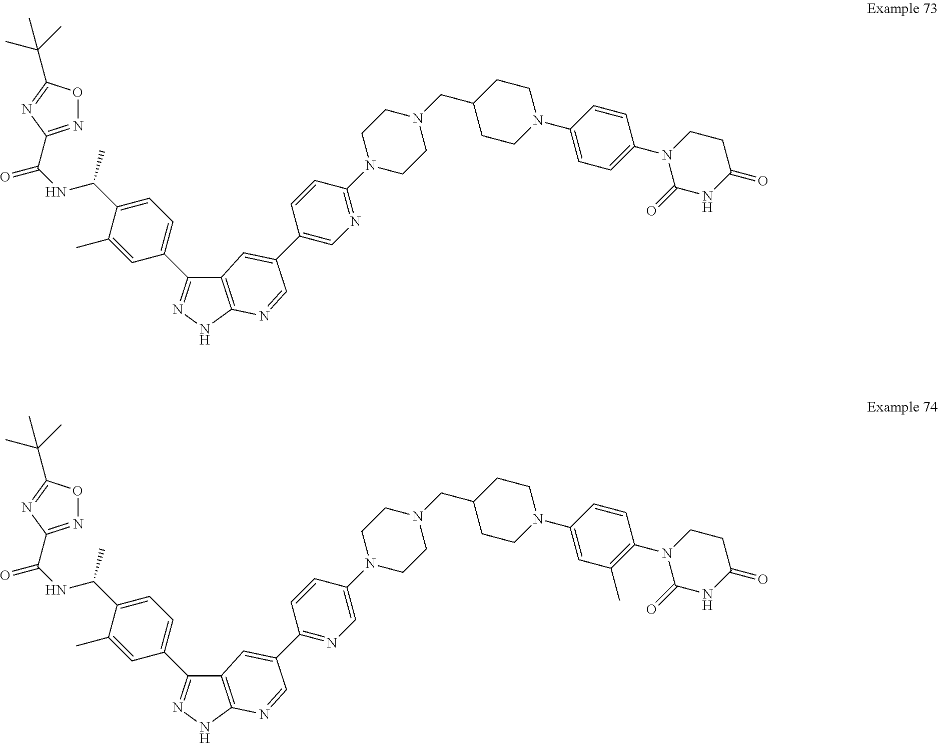 [SMILES: Cc1cc(-c2n[nH]c3ncc(-c4ccc(N5CCN(CC6CCN(c7ccc(N8CCC(=O)NC8=O)cc7)CC6)CC5)nc4)cc23)ccc1[C@@H](C)NC(=O)c1noc(C(C)(C)C)n1 ; Cc1cc(-c2n[nH]c3ncc(-c4ccc(N5CCN(CC6CCN(c7ccc(N8CCC(=O)NC8=O)c(C)c7)CC6)CC5)cn4)cc23)ccc1[C@@H](C)NC(=O)c1noc(C(C)(C)C)n1]

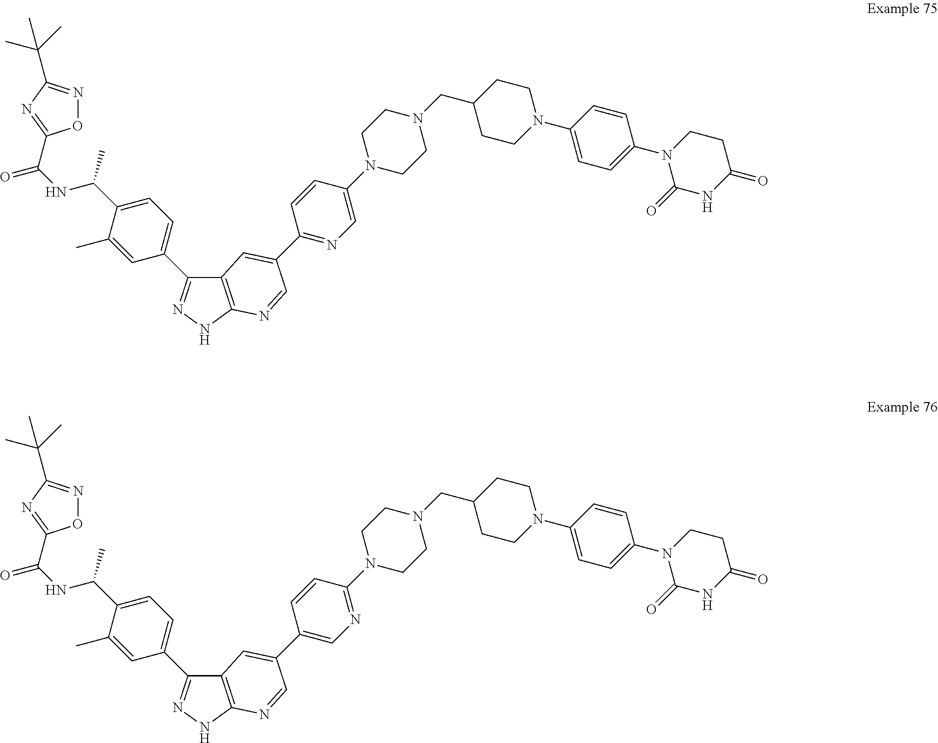 [SMILES: Cc1cc(-c2n[nH]c3ncc(-c4ccc(N5CCN(CC6CCN(c7ccc(N8CCC(=O)NC8=O)cc7)CC6)CC5)cn4)cc23)ccc1[C@@H](C)NC(=O)c1nc(C(C)(C)C)no1 ; Cc1cc(-c2n[nH]c3ncc(-c4ccc(N5CCN(CC6CCN(c7ccc(N8CCC(=O)NC8=O)cc7)CC6)CC5)nc4)cc23)ccc1[C@@H](C)NC(=O)c1nc(C(C)(C)C)no1]

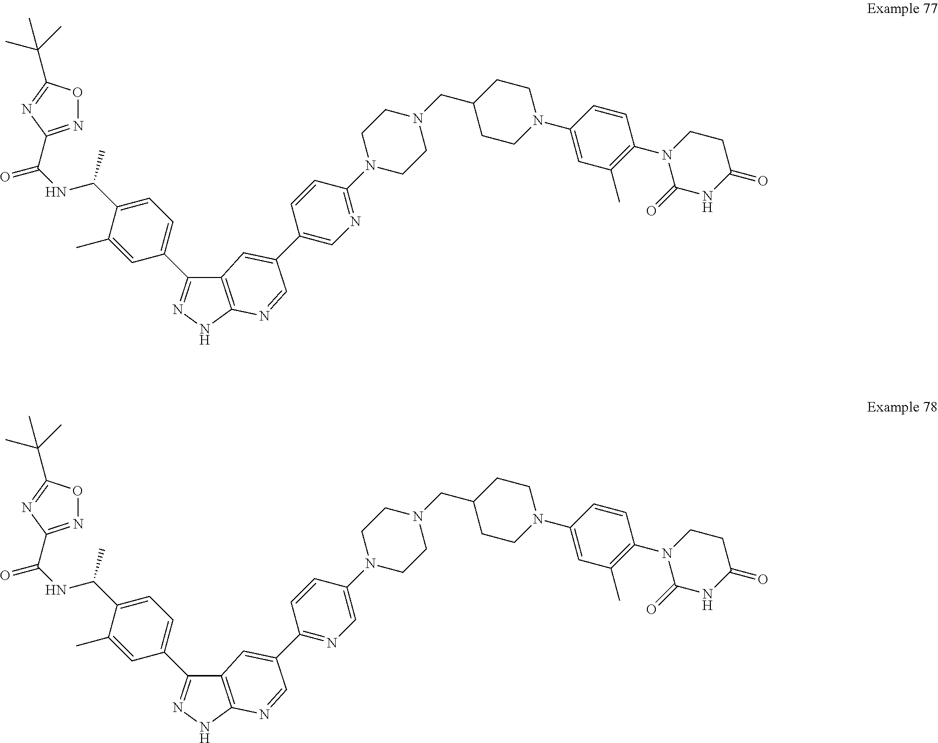 [SMILES: Cc1cc(-c2n[nH]c3ncc(-c4ccc(N5CCN(CC6CCN(c7ccc(N8CCC(=O)NC8=O)c(C)c7)CC6)CC5)nc4)cc23)ccc1[C@@H](C)NC(=O)c1noc(C(C)(C)C)n1 ; Cc1cc(-c2n[nH]c3ncc(-c4ccc(N5CCN(CC6CCN(c7ccc(N8CCC(=O)NC8=O)c(C)c7)CC6)CC5)cn4)cc23)ccc1[C@@H](C)NC(=O)c1noc(C(C)(C)C)n1]

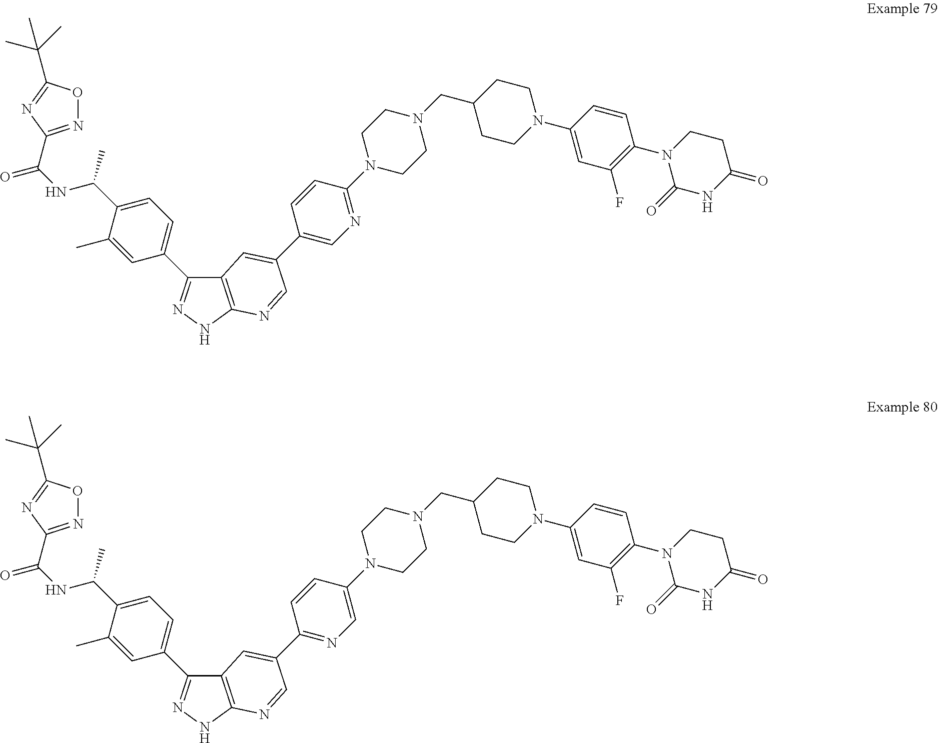 [SMILES: Cc1cc(-c2n[nH]c3ncc(-c4ccc(N5CCN(CC6CCN(c7ccc(N8CCC(=O)NC8=O)c(F)c7)CC6)CC5)nc4)cc23)ccc1[C@@H](C)NC(=O)c1noc(C(C)(C)C)n1 ; Cc1cc(-c2n[nH]c3ncc(-c4ccc(N5CCN(CC6CCN(c7ccc(N8CCC(=O)NC8=O)c(F)c7)CC6)CC5)cn4)cc23)ccc1[C@@H](C)NC(=O)c1noc(C(C)(C)C)n1]

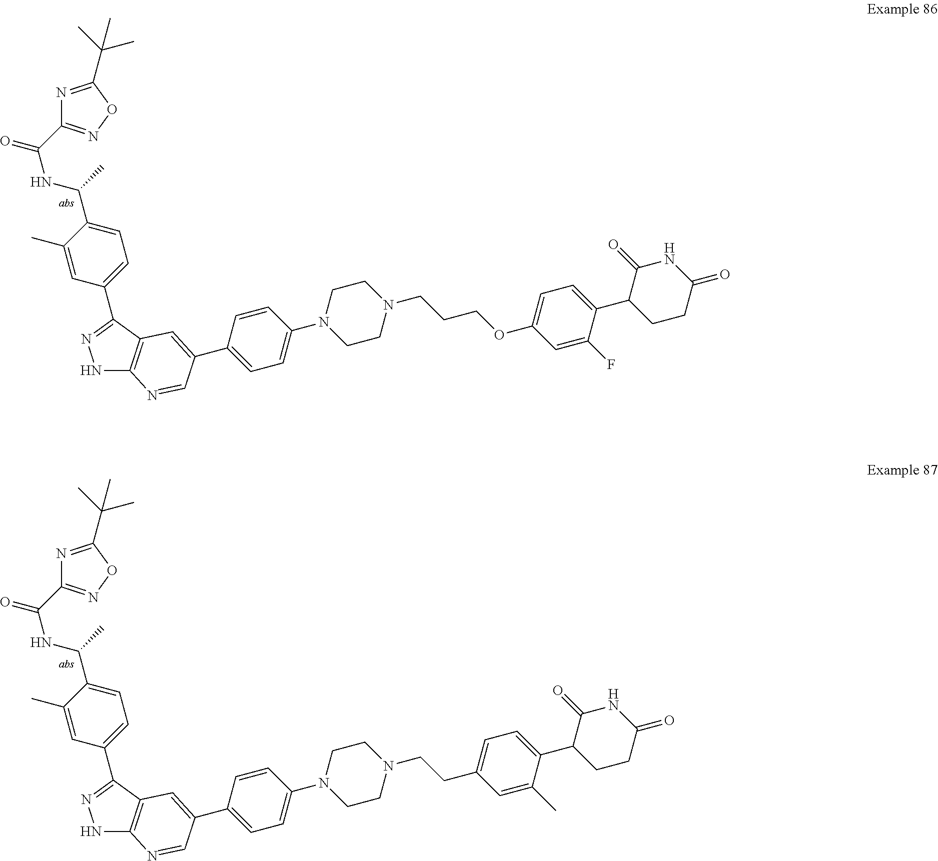 [SMILES: Cc1cc(-c2n[nH]c3ncc(-c4ccc(N5CCN(CCCOc6ccc(C7CCC(=O)NC7=O)c(F)c6)CC5)cc4)cc23)ccc1[C@@H](C)NC(=O)c1noc(C(C)(C)C)n1 ; Cc1cc(CCN2CCN(c3ccc(-c4cnc5[nH]nc(-c6ccc([C@@H](C)NC(=O)c7noc(C(C)(C)C)n7)c(C)c6)c5c4)cc3)CC2)ccc1C1CCC(=O)NC1=O]

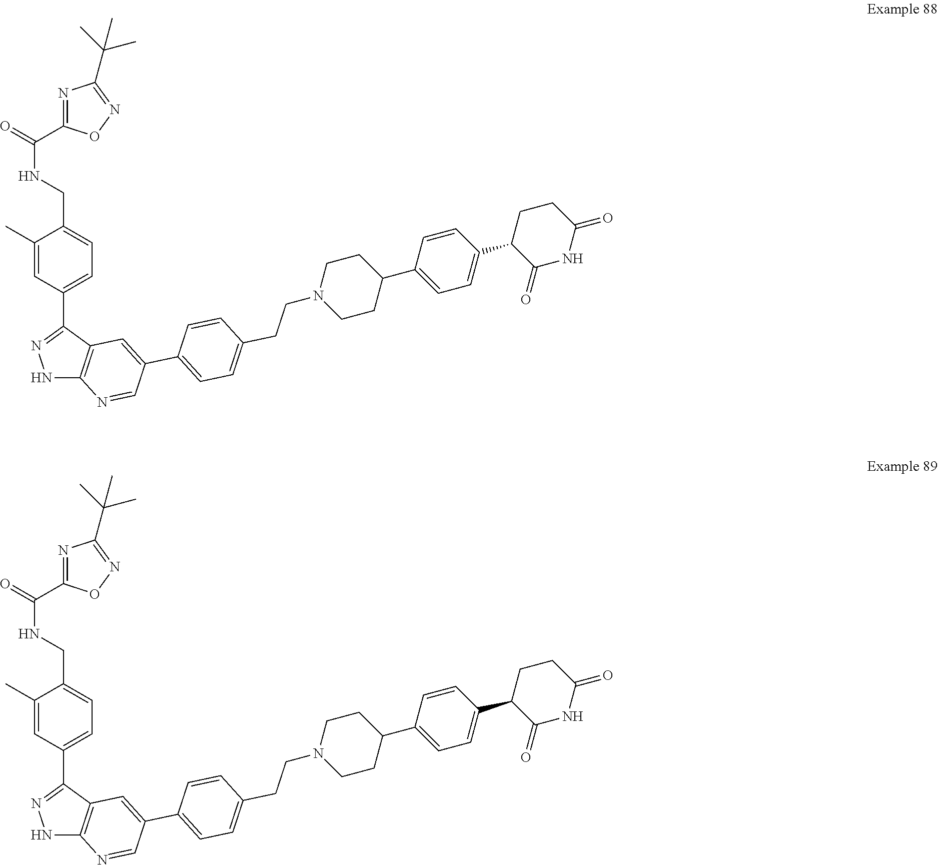 [SMILES: Cc1cc(-c2n[nH]c3ncc(-c4ccc(CCN5CCC(c6ccc([C@@H]7CCC(=O)NC7=O)cc6)CC5)cc4)cc23)ccc1CNC(=O)c1nc(C(C)(C)C)no1 ; Cc1cc(-c2n[nH]c3ncc(-c4ccc(CCN5CCC(c6ccc([C@H]7CCC(=O)NC7=O)cc6)CC5)cc4)cc23)ccc1CNC(=O)c1nc(C(C)(C)C)no1]

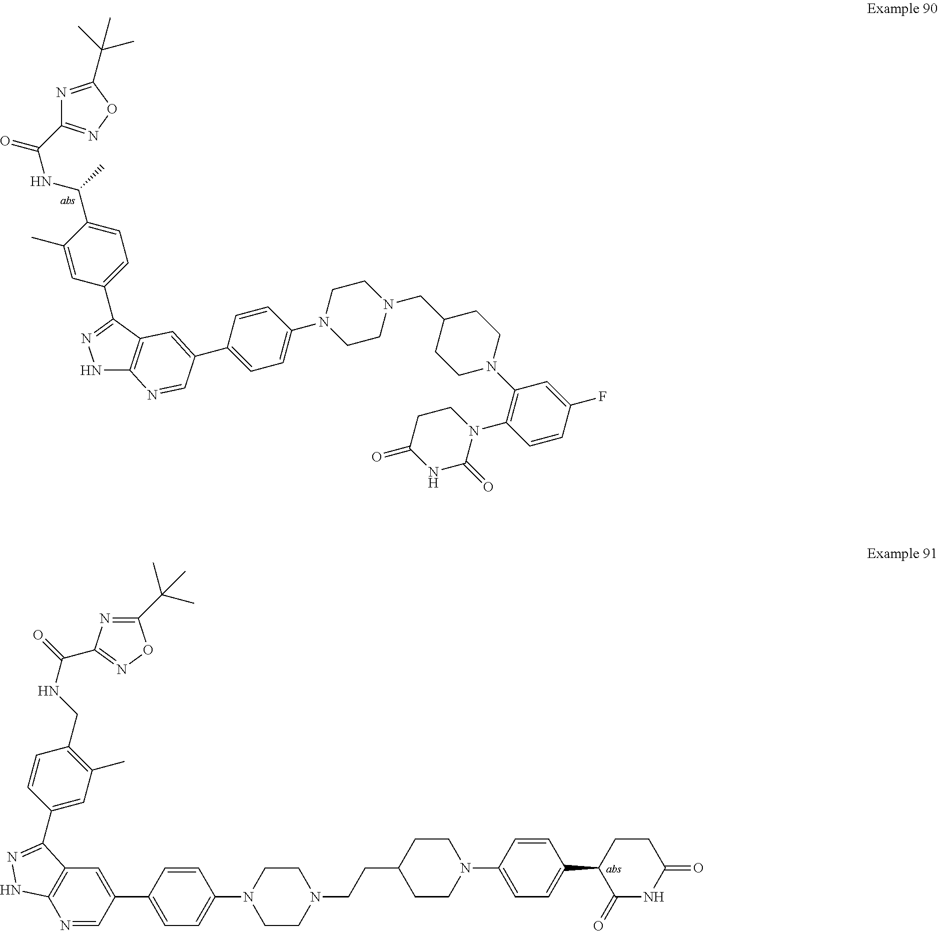 [SMILES: Cc1cc(-c2n[nH]c3ncc(-c4ccc(N5CCN(CC6CCN(c7cc(F)ccc7N7CCC(=O)NC7=O)CC6)CC5)cc4)cc23)ccc1[C@@H](C)NC(=O)c1noc(C(C)(C)C)n1 ; Cc1cc(-c2n[nH]c3ncc(-c4ccc(N5CCN(CCC6CCN(c7ccc([C@H]8CCC(=O)NC8=O)cc7)CC6)CC5)cc4)cc23)ccc1CNC(=O)c1noc(C(C)(C)C)n1]

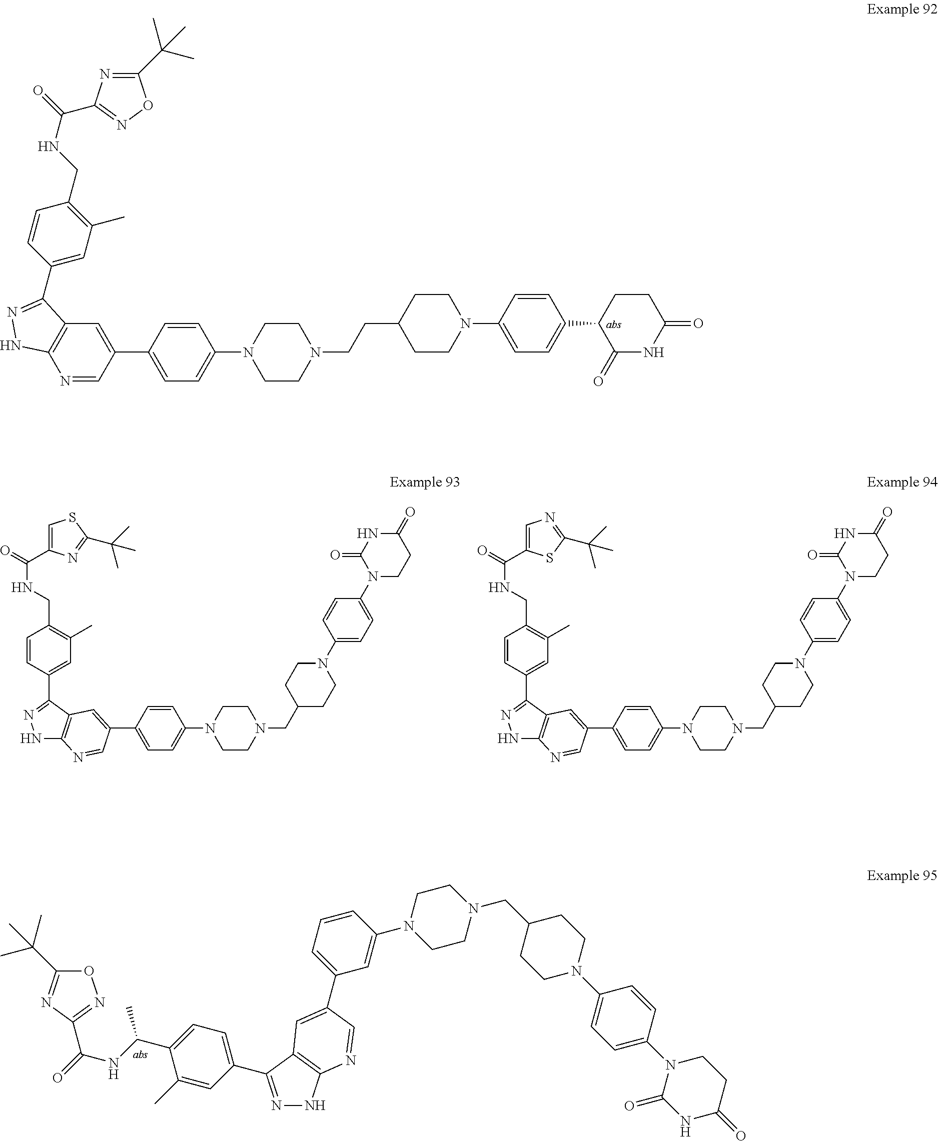 [SMILES: Cc1cc(-c2n[nH]c3ncc(-c4ccc(N5CCN(CCC6CCN(c7ccc([C@@H]8CCC(=O)NC8=O)cc7)CC6)CC5)cc4)cc23)ccc1CNC(=O)c1noc(C(C)(C)C)n1 ; Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1csc(C(C)(C)C)n1 ; Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1cnc(C(C)(C)C)s1 ; Cc1cc(-c2n[nH]c3ncc(-c4cccc(N5CCN(CC6CCN(c7ccc(N8CCC(=O)NC8=O)cc7)CC6)CC5)c4)cc23)ccc1[C@@H](C)NC(=O)c1noc(C(C)(C)C)n1]

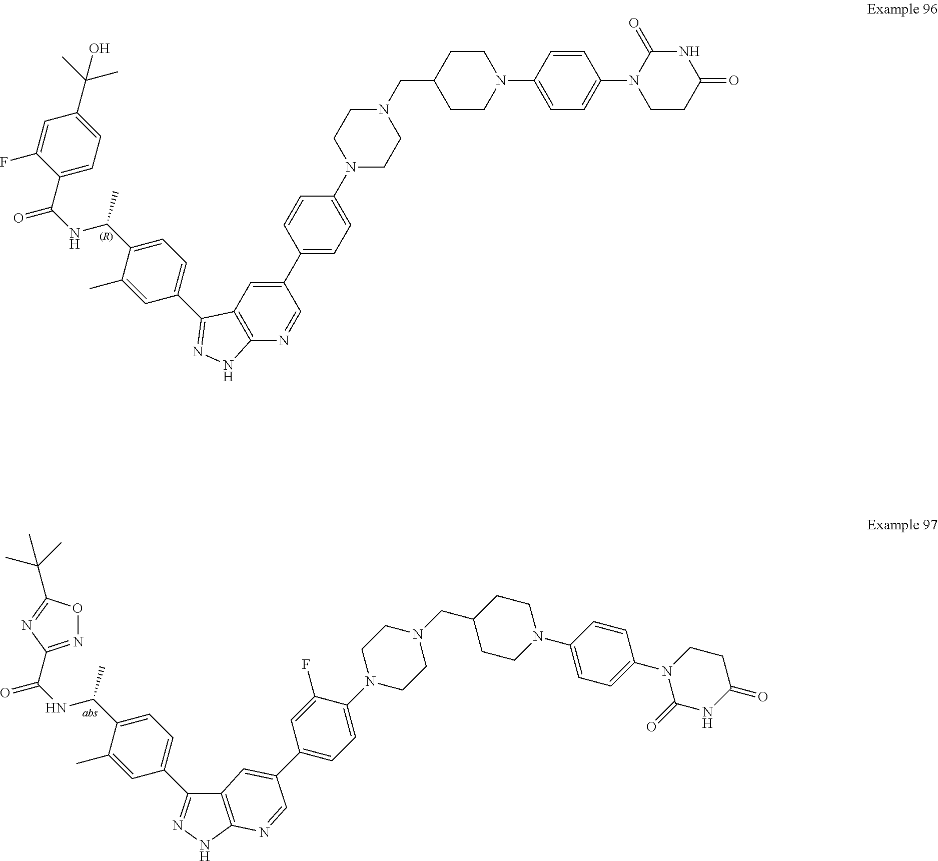

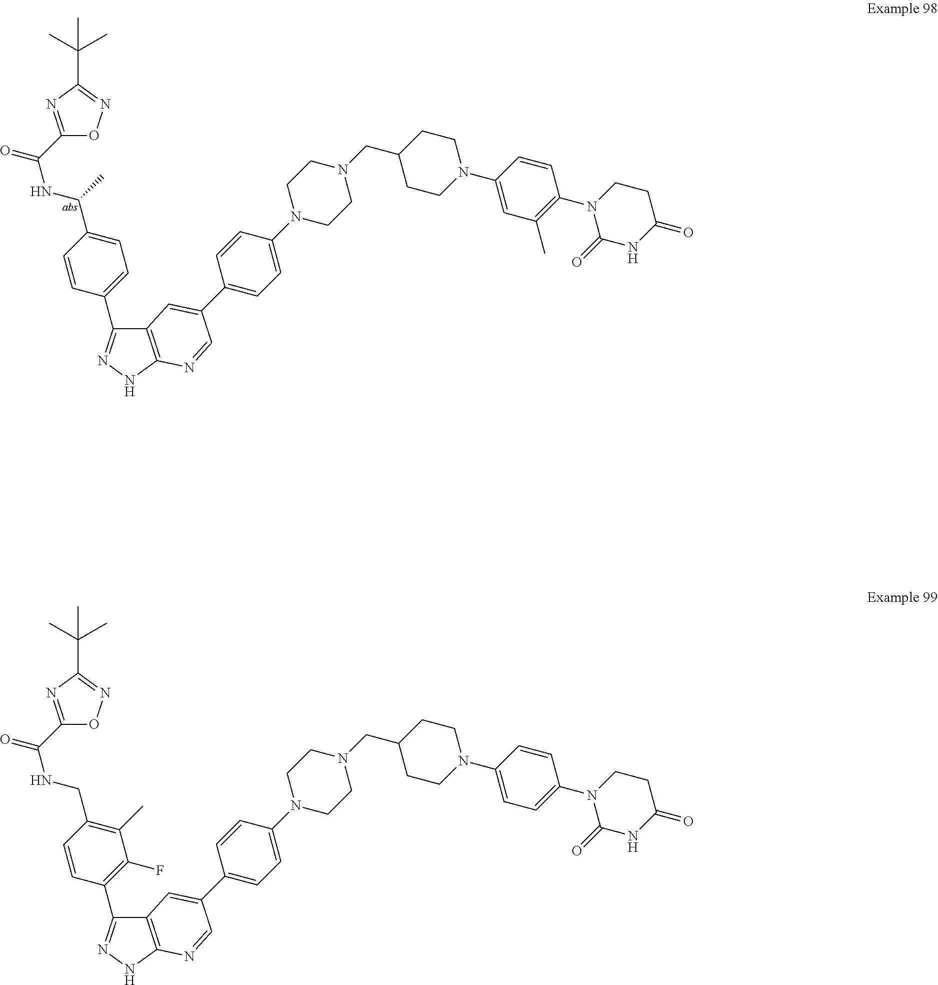 [SMILES: Cc1cc(N2CCC(CN3CCN(c4ccc(-c5cnc6[nH]nc(-c7ccc([C@@H](C)NC(=O)c8nc(C(C)(C)C)no8)cc7)c6c5)cc4)CC3)CC2)ccc1N1CCC(=O)NC1=O ; Cc1c(CNC(=O)c2nc(C(C)(C)C)no2)ccc(-c2n[nH]c3ncc(-c4ccc(N5CCN(CC6CCN(c7ccc(N8CCC(=O)NC8=O)cc7)CC6)CC5)cc4)cc23)c1F]

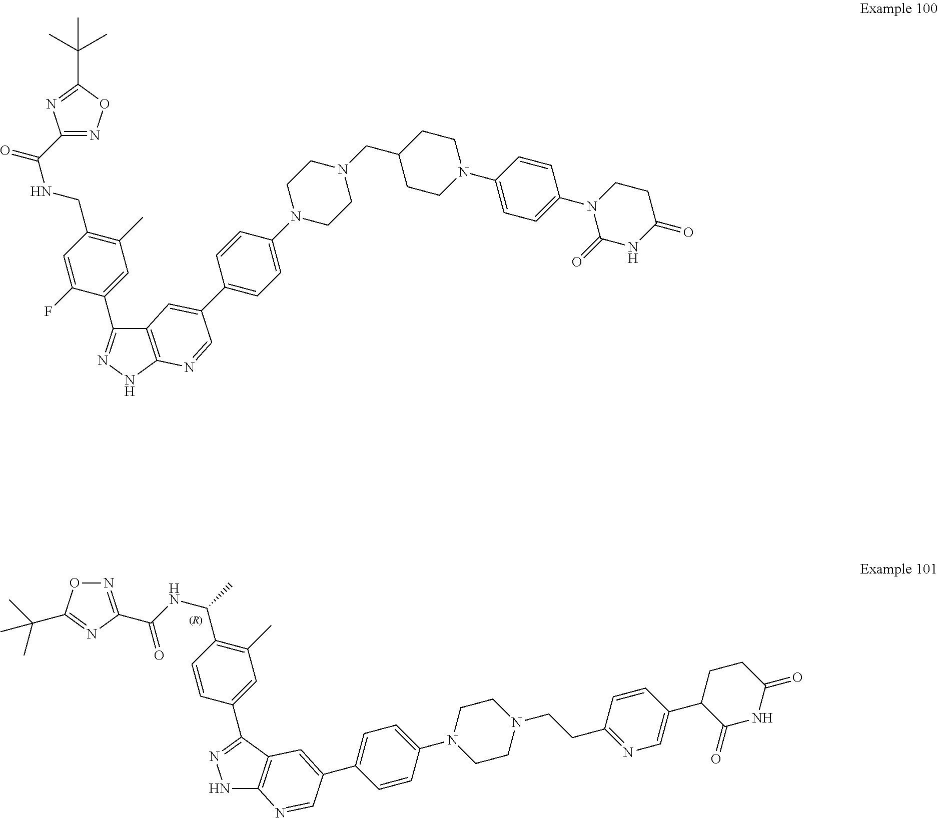

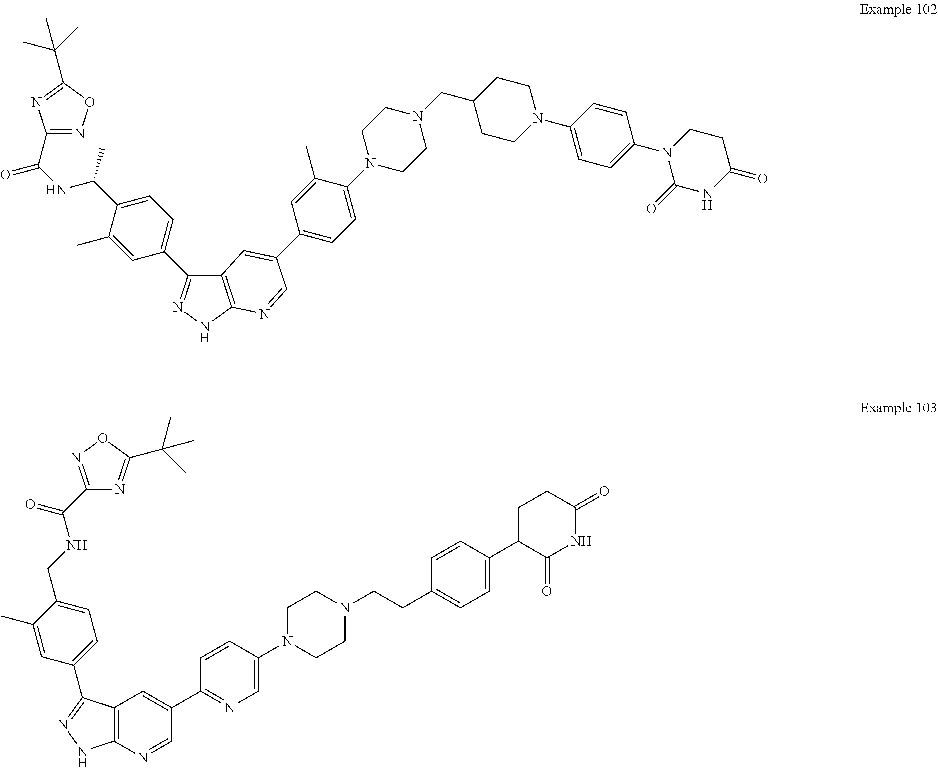 [SMILES: Cc1cc(-c2n[nH]c3ncc(-c4ccc(N5CCN(CC6CCN(c7ccc(N8CCC(=O)NC8=O)cc7)CC6)CC5)c(C)c4)cc23)ccc1[C@@H](C)NC(=O)c1noc(C(C)(C)C)n1 ; Cc1cc(-c2n[nH]c3ncc(-c4ccc(N5CCN(CCc6ccc(C7CCC(=O)NC7=O)cc6)CC5)cn4)cc23)ccc1CNC(=O)c1noc(C(C)(C)C)n1]

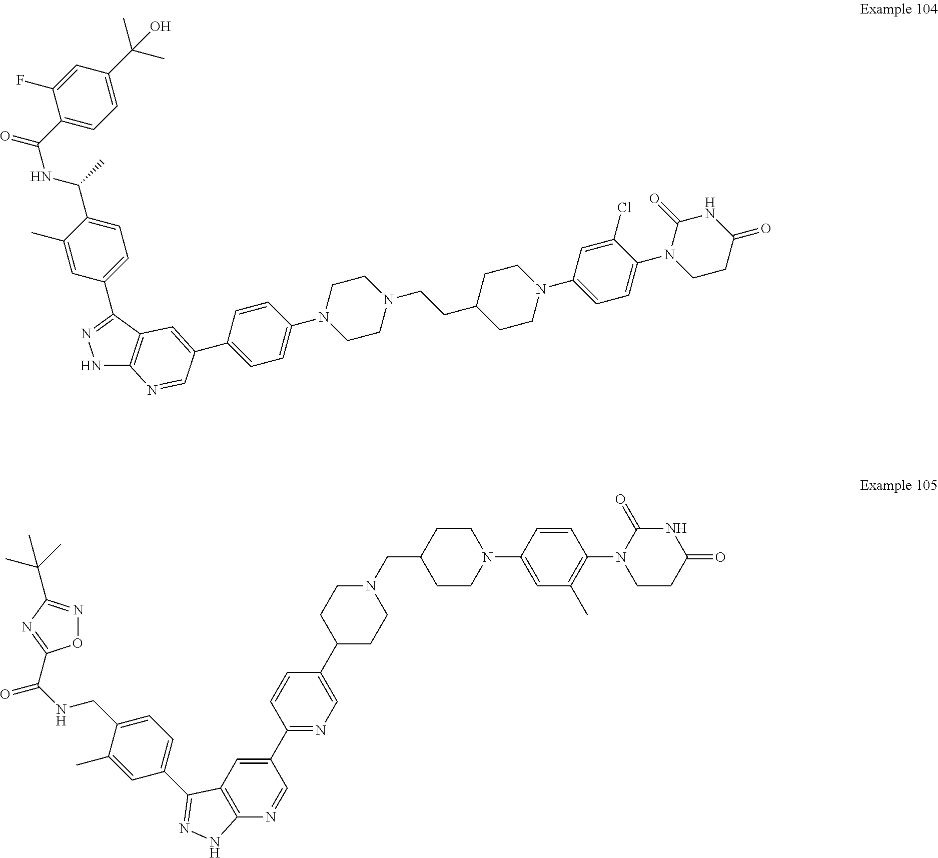 [SMILES: Cc1cc(-c2n[nH]c3ncc(-c4ccc(N5CCN(CCC6CCN(c7ccc(N8CCC(=O)NC8=O)c(Cl)c7)CC6)CC5)cc4)cc23)ccc1[C@@H](C)NC(=O)c1ccc(C(C)(C)O)cc1F ; Cc1cc(-c2n[nH]c3ncc(-c4ccc(C5CCN(CC6CCN(c7ccc(N8CCC(=O)NC8=O)c(C)c7)CC6)CC5)cn4)cc23)ccc1CNC(=O)c1nc(C(C)(C)C)no1]

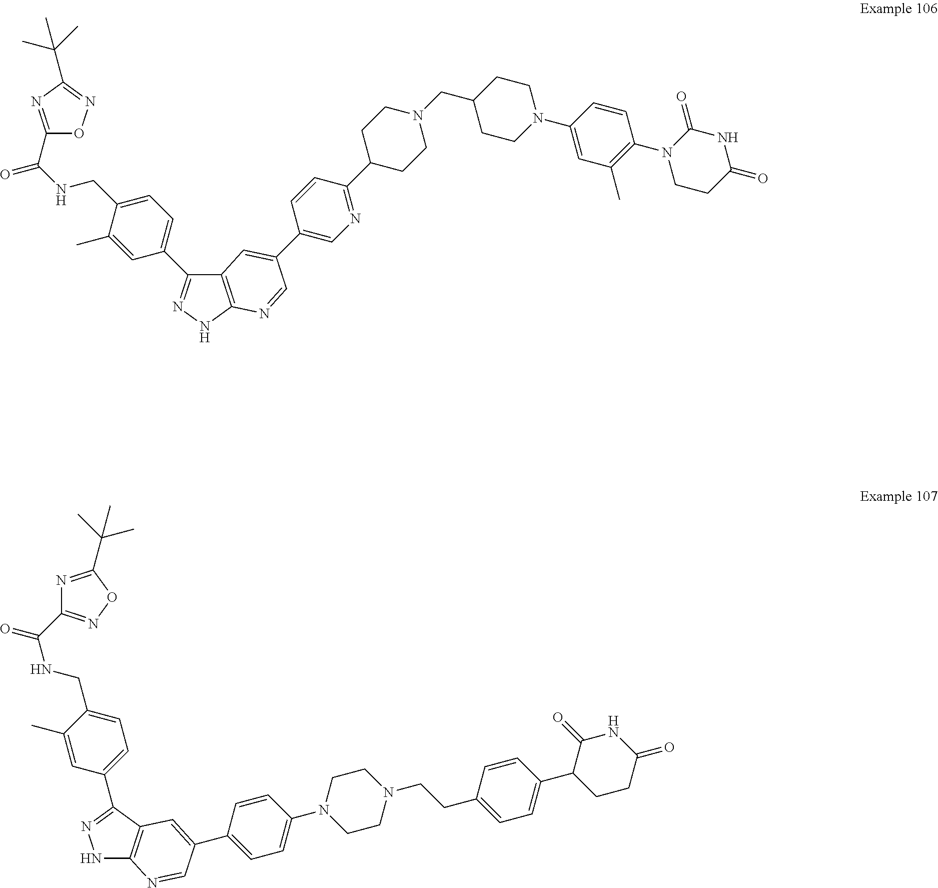 [SMILES: Cc1cc(-c2n[nH]c3ncc(-c4ccc(C5CCN(CC6CCN(c7ccc(N8CCC(=O)NC8=O)c(C)c7)CC6)CC5)nc4)cc23)ccc1CNC(=O)c1nc(C(C)(C)C)no1 ; Cc1cc(-c2n[nH]c3ncc(-c4ccc(N5CCN(CCc6ccc(C7CCC(=O)NC7=O)cc6)CC5)cc4)cc23)ccc1CNC(=O)c1noc(C(C)(C)C)n1]

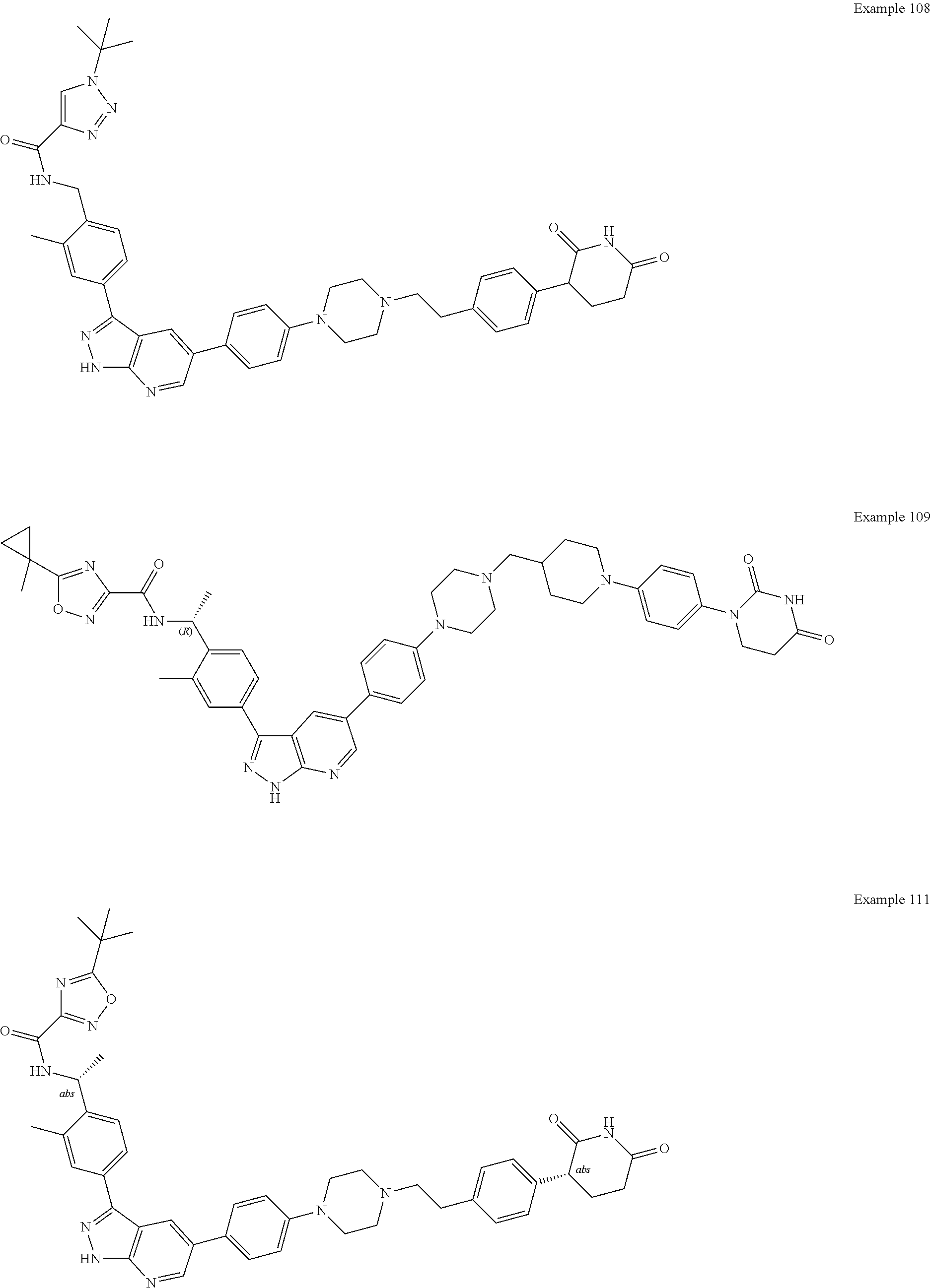

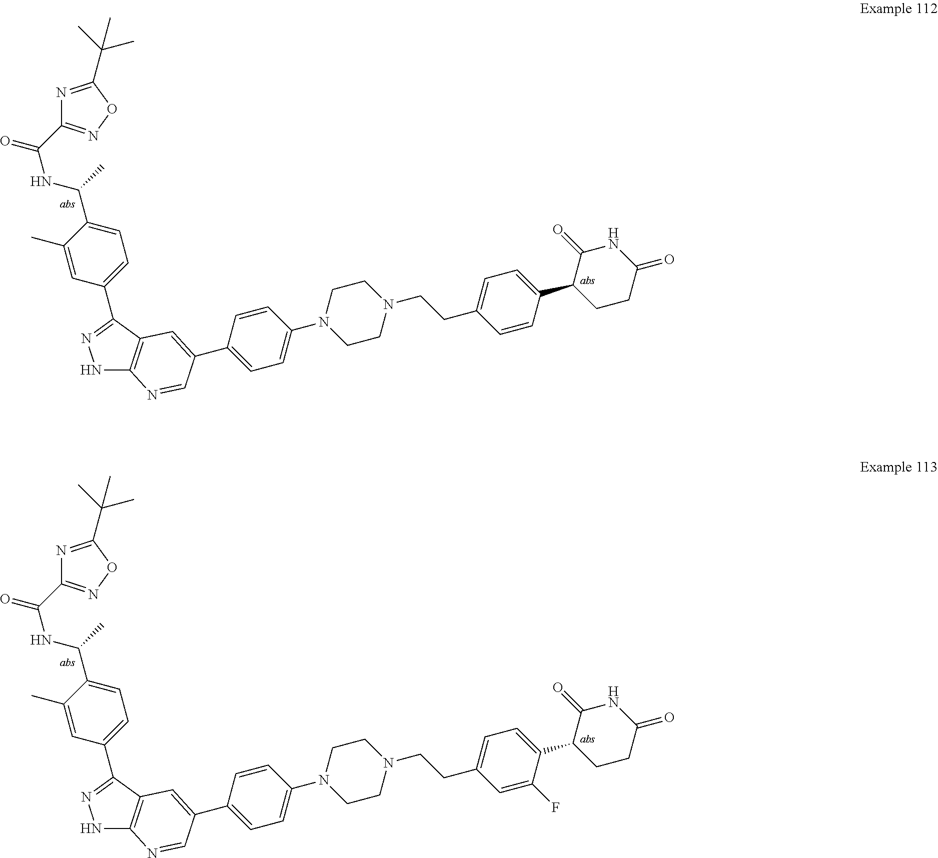 [SMILES: Cc1cc(-c2n[nH]c3ncc(-c4ccc(N5CCN(CCc6ccc([C@@H]7CCC(=O)NC7=O)cc6)CC5)cc4)cc23)ccc1[C@@H](C)NC(=O)c1noc(C(C)(C)C)n1 ; Cc1cc(-c2n[nH]c3ncc(-c4ccc(N5CCN(CCc6ccc([C@H]7CCC(=O)NC7=O)c(F)c6)CC5)cc4)cc23)ccc1[C@@H](C)NC(=O)c1noc(C(C)(C)C)n1]

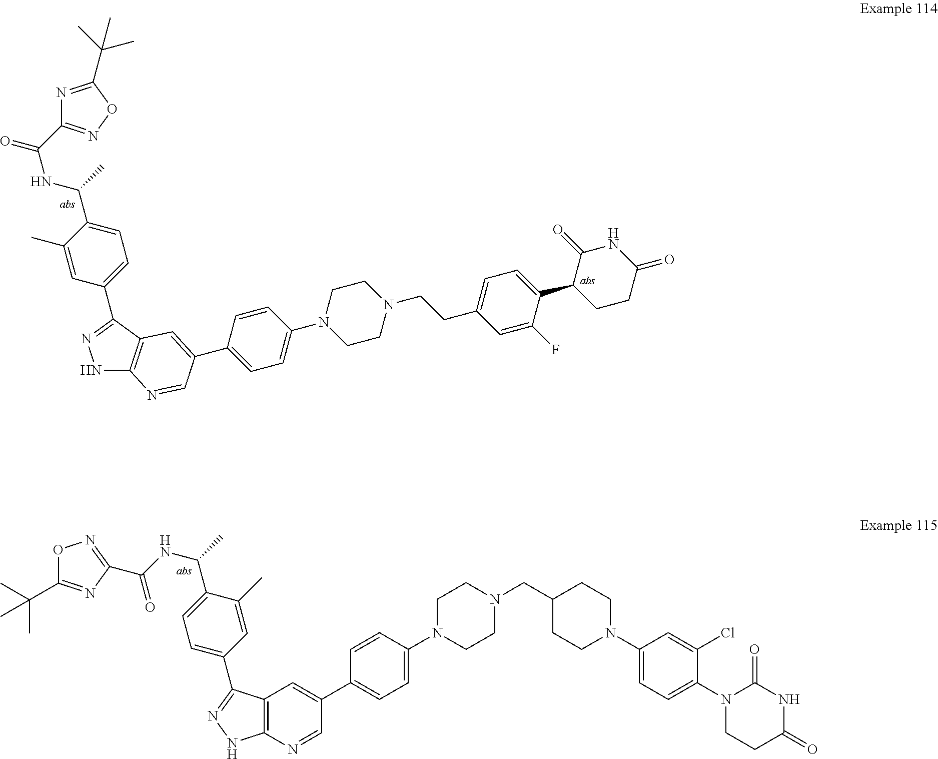 [SMILES: Cc1cc(-c2n[nH]c3ncc(-c4ccc(N5CCN(CCc6ccc([C@@H]7CCC(=O)NC7=O)c(F)c6)CC5)cc4)cc23)ccc1[C@@H](C)NC(=O)c1noc(C(C)(C)C)n1 ; Cc1cc(-c2n[nH]c3ncc(-c4ccc(N5CCN(CC6CCN(c7ccc(N8CCC(=O)NC8=O)c(Cl)c7)CC6)CC5)cc4)cc23)ccc1[C@@H](C)NC(=O)c1noc(C(C)(C)C)n1]

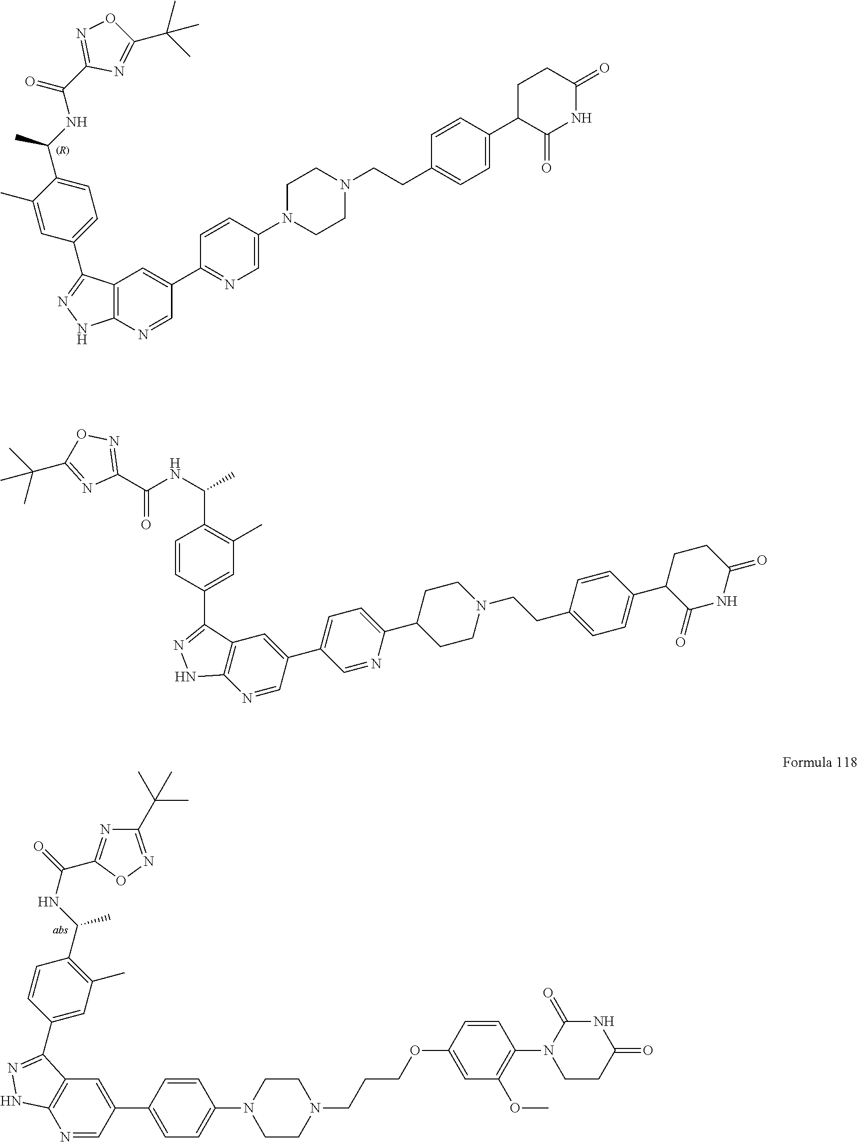

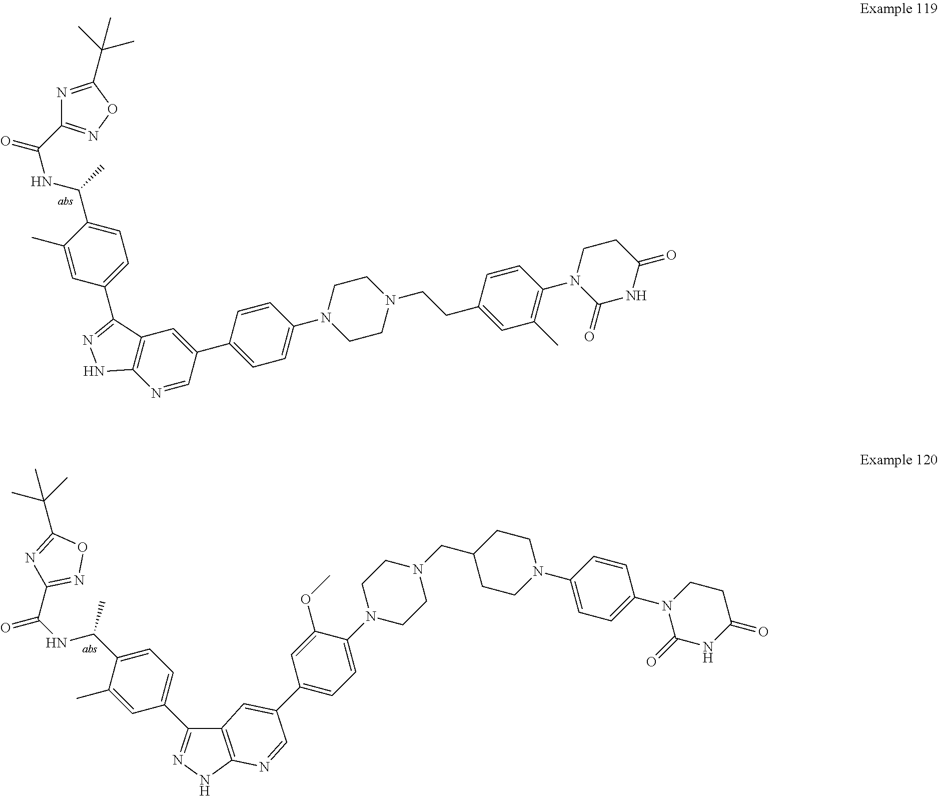

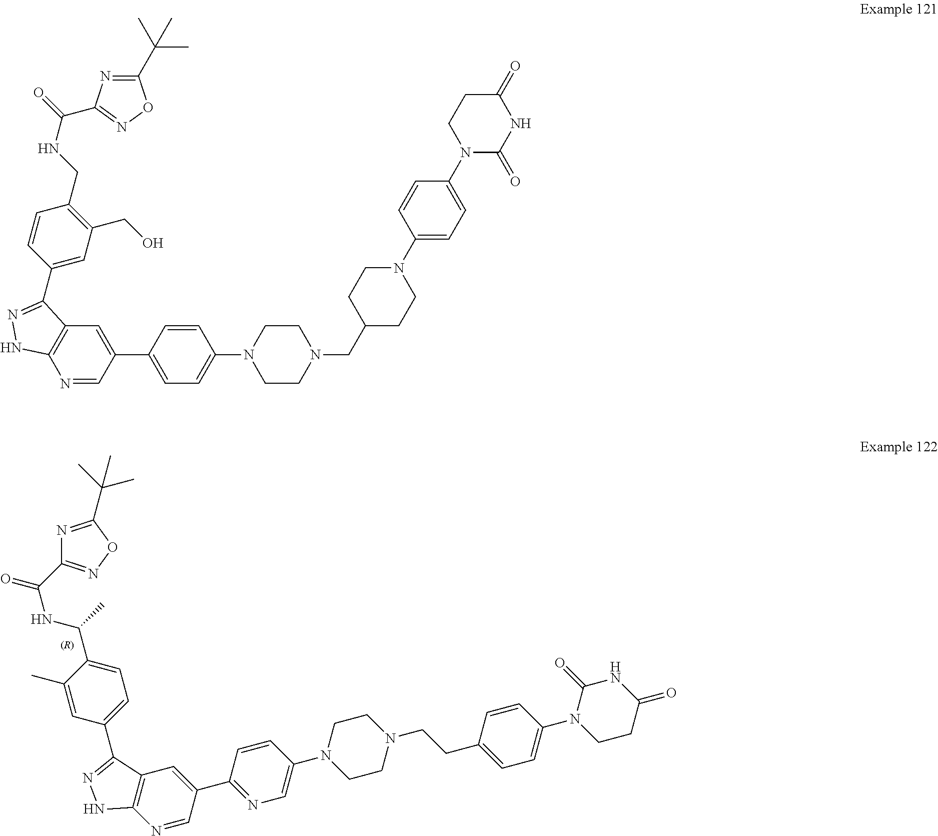

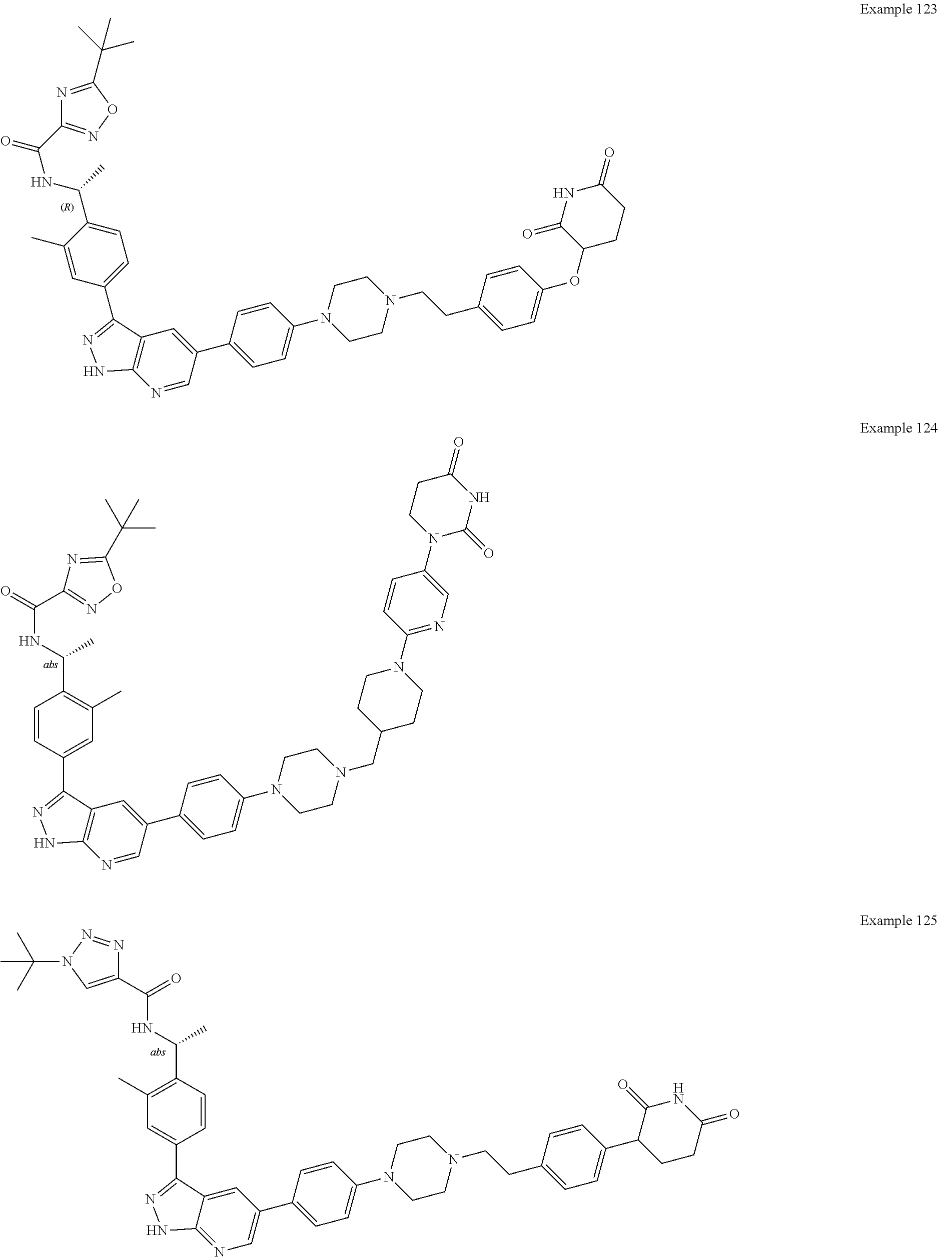

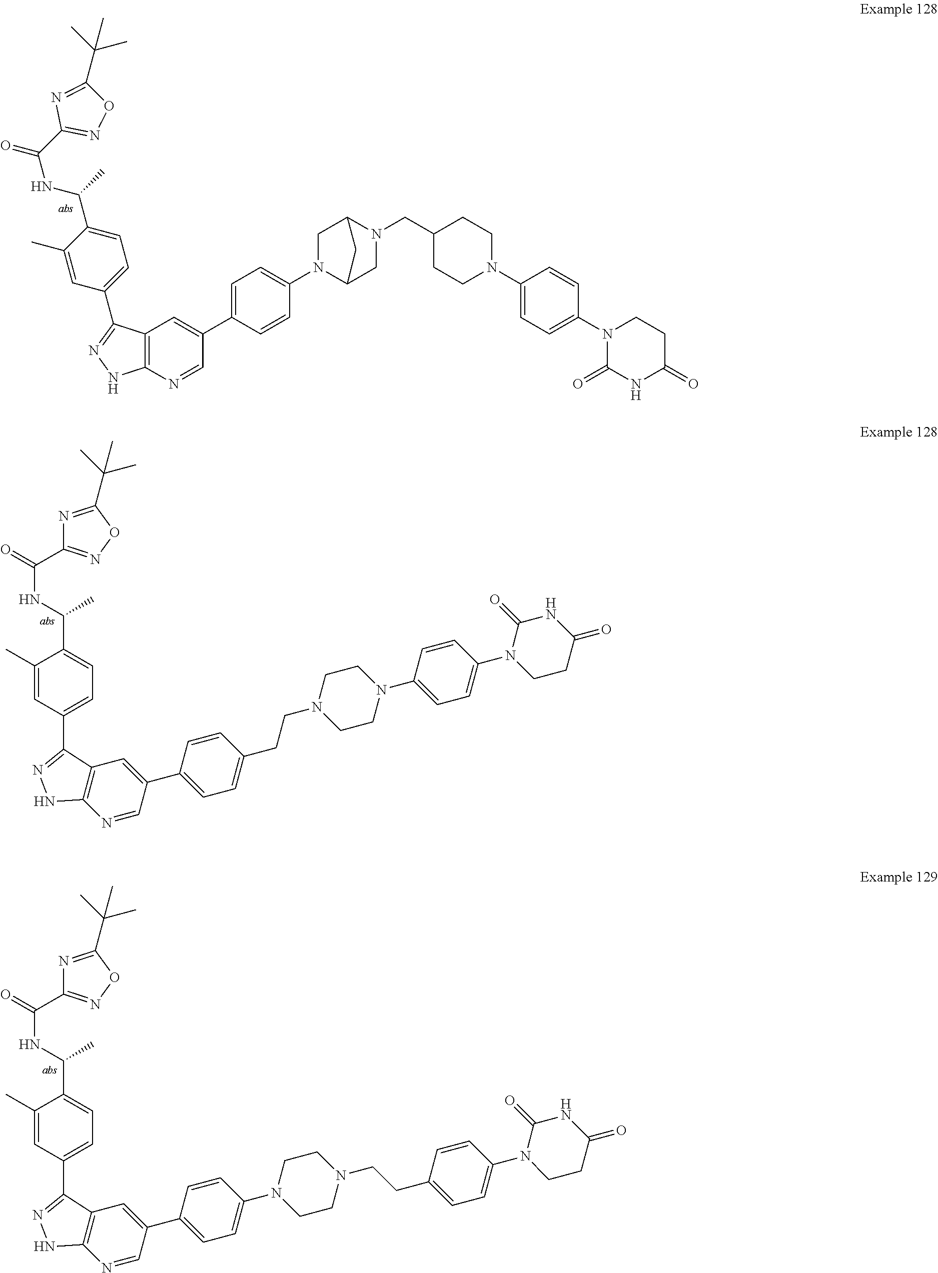

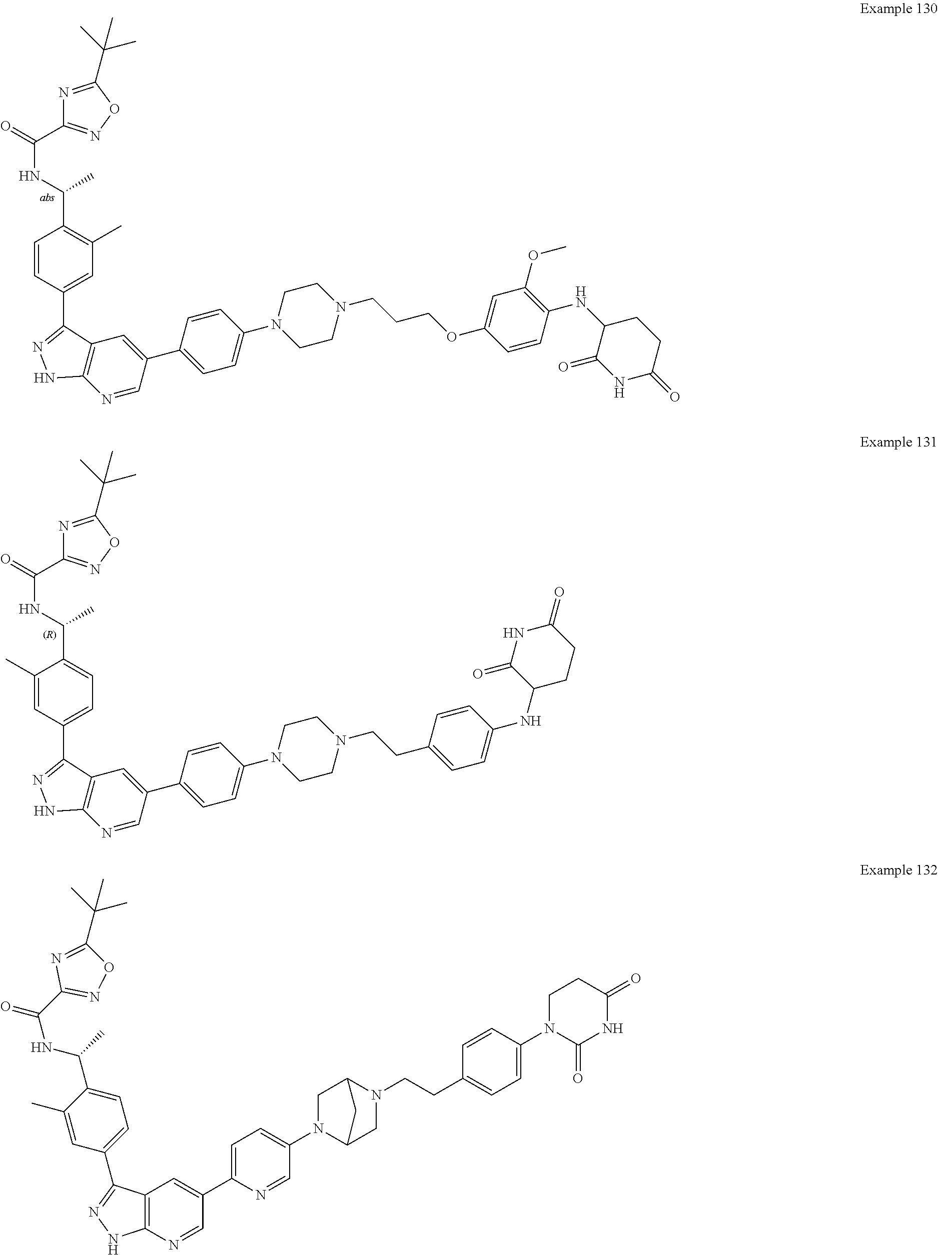

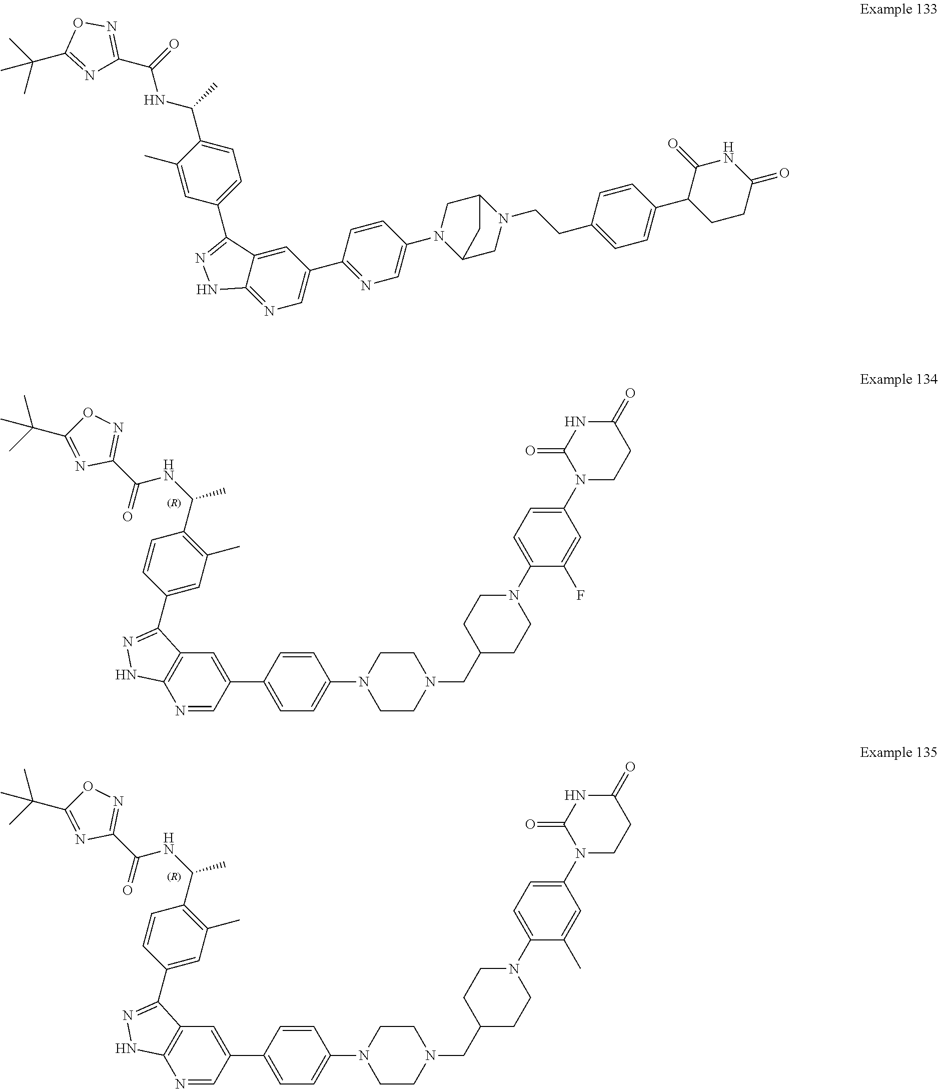

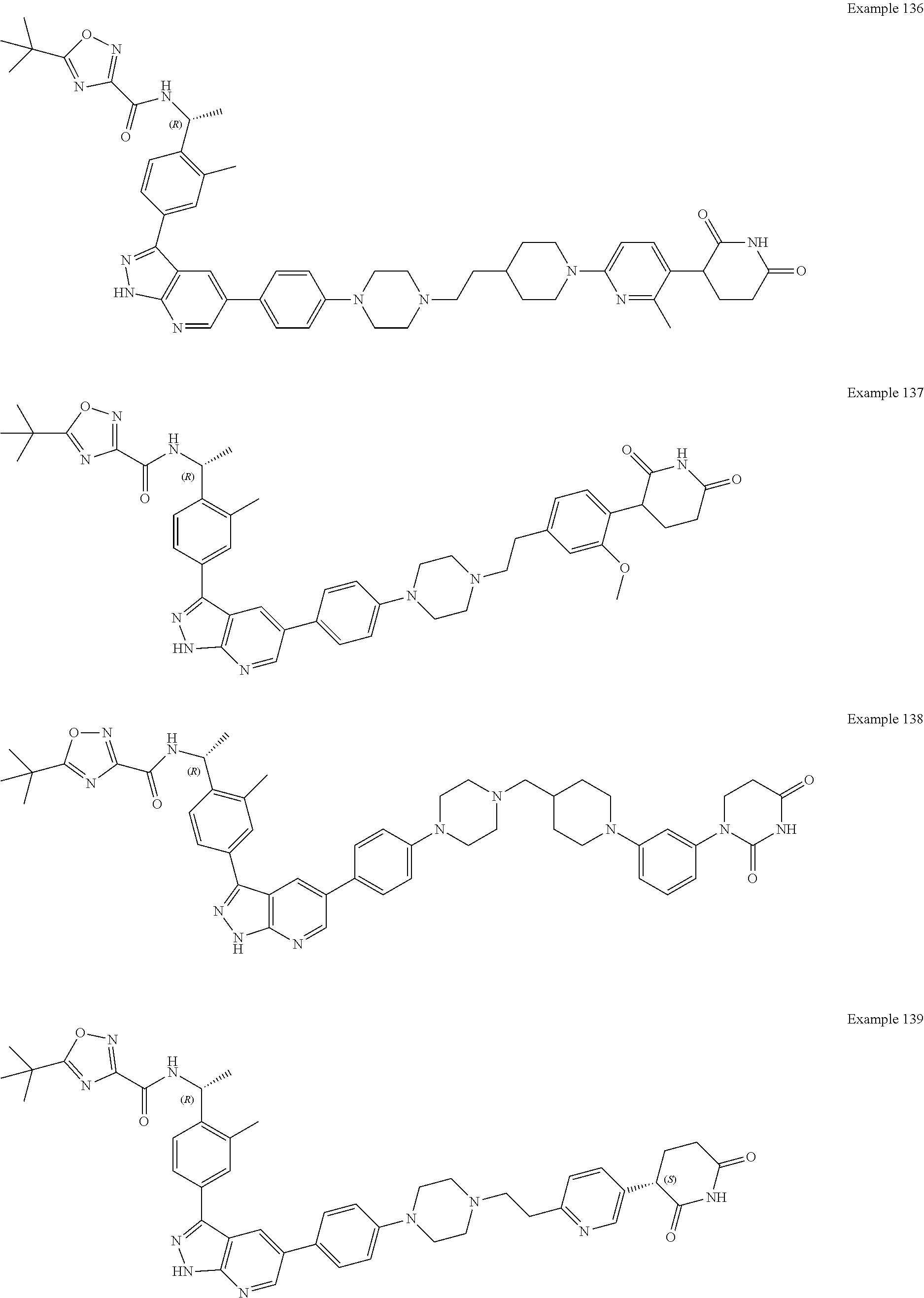

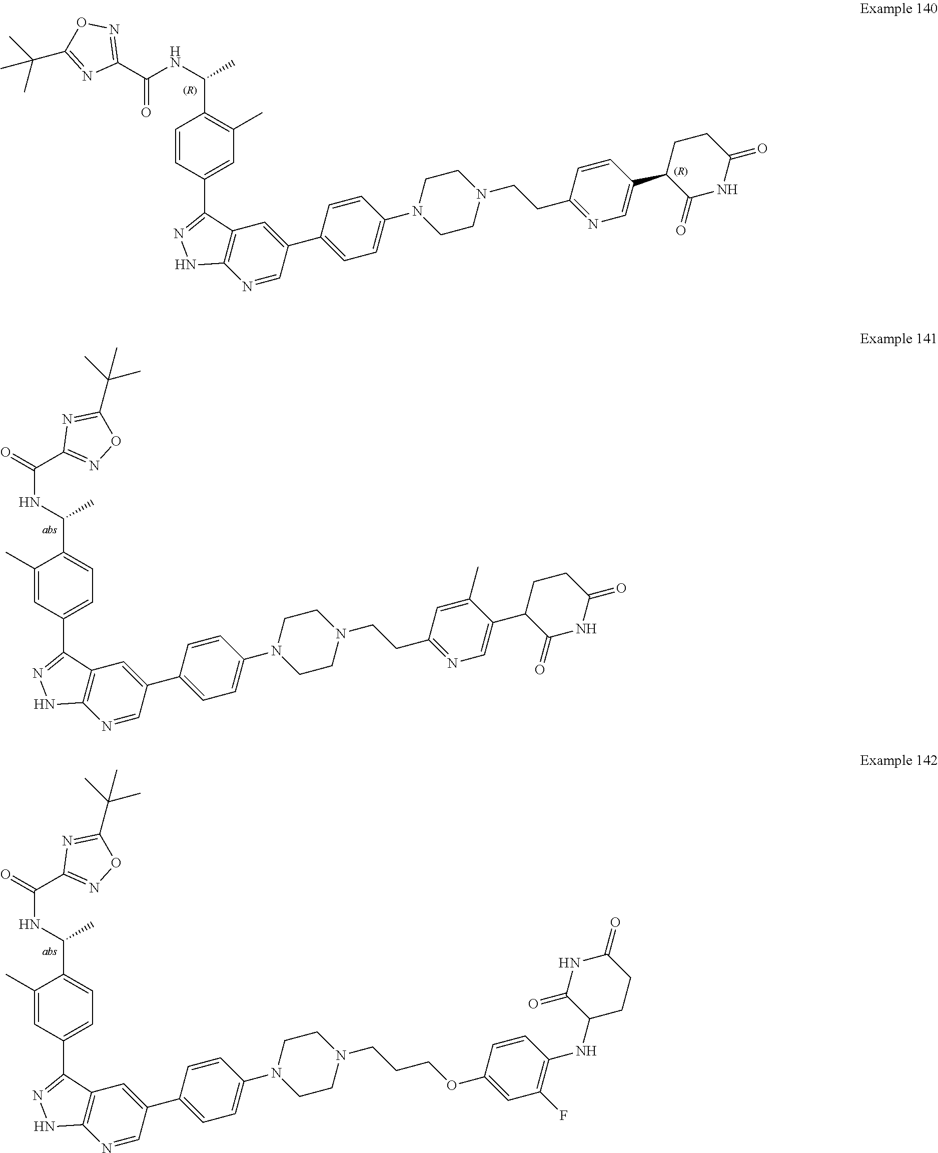

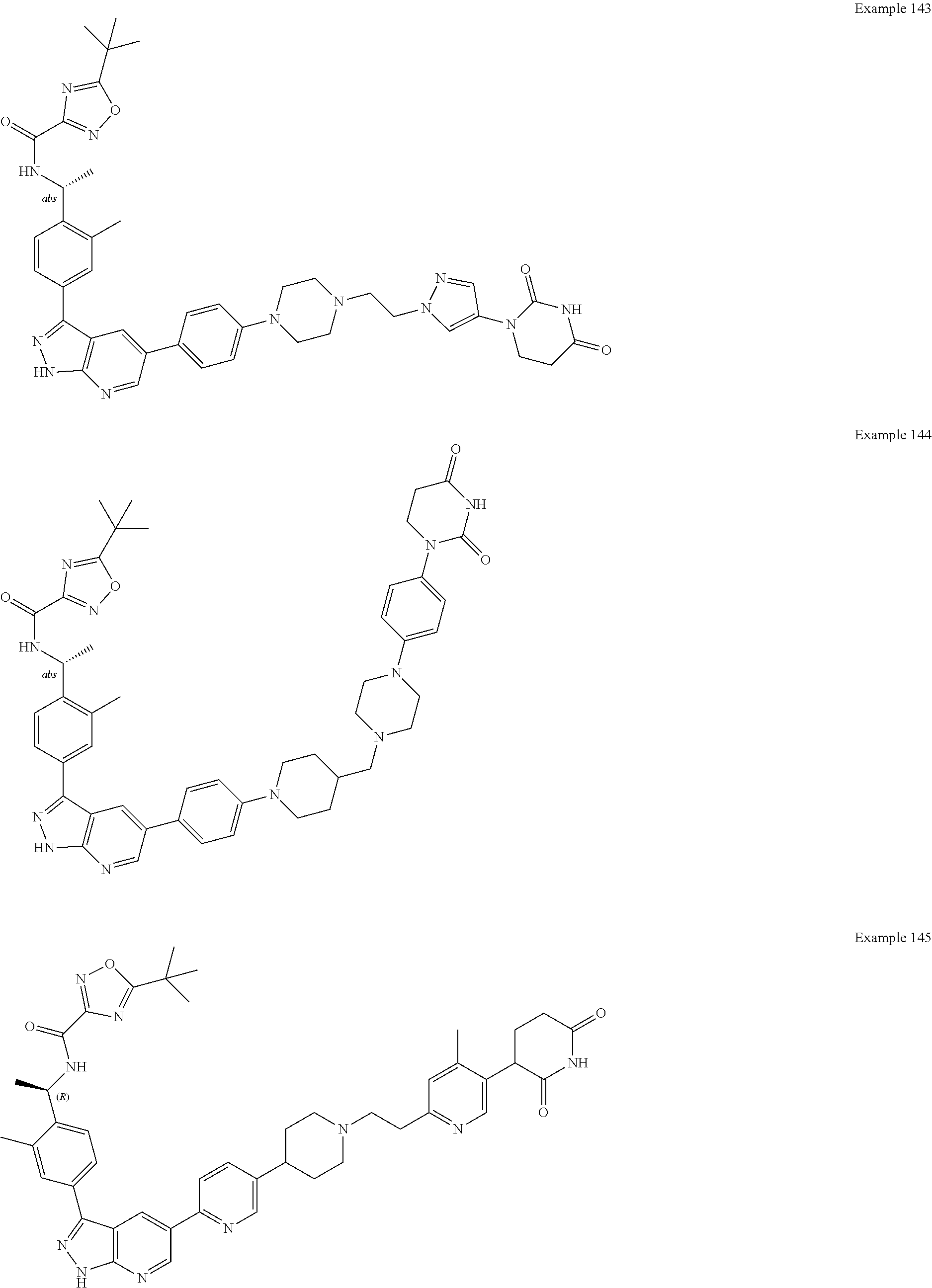

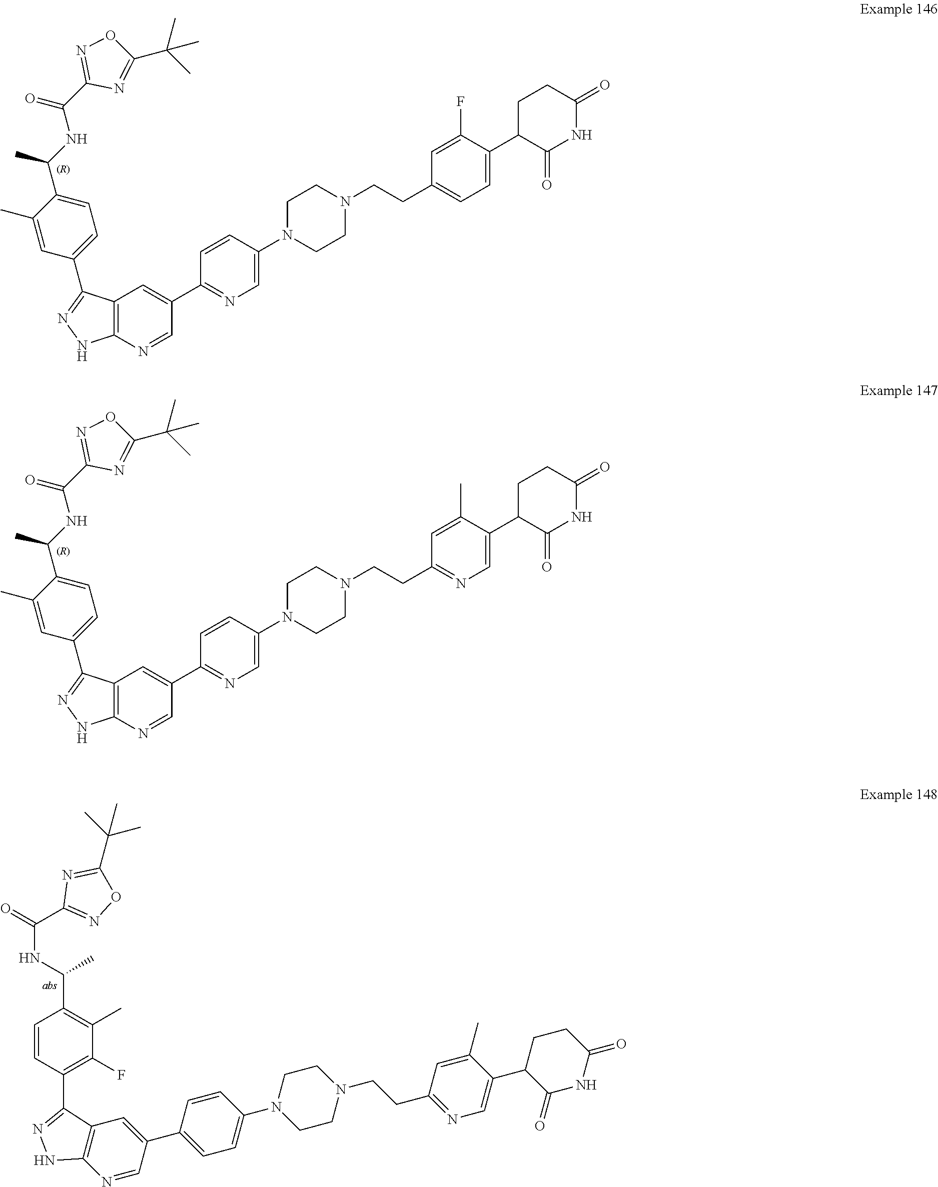

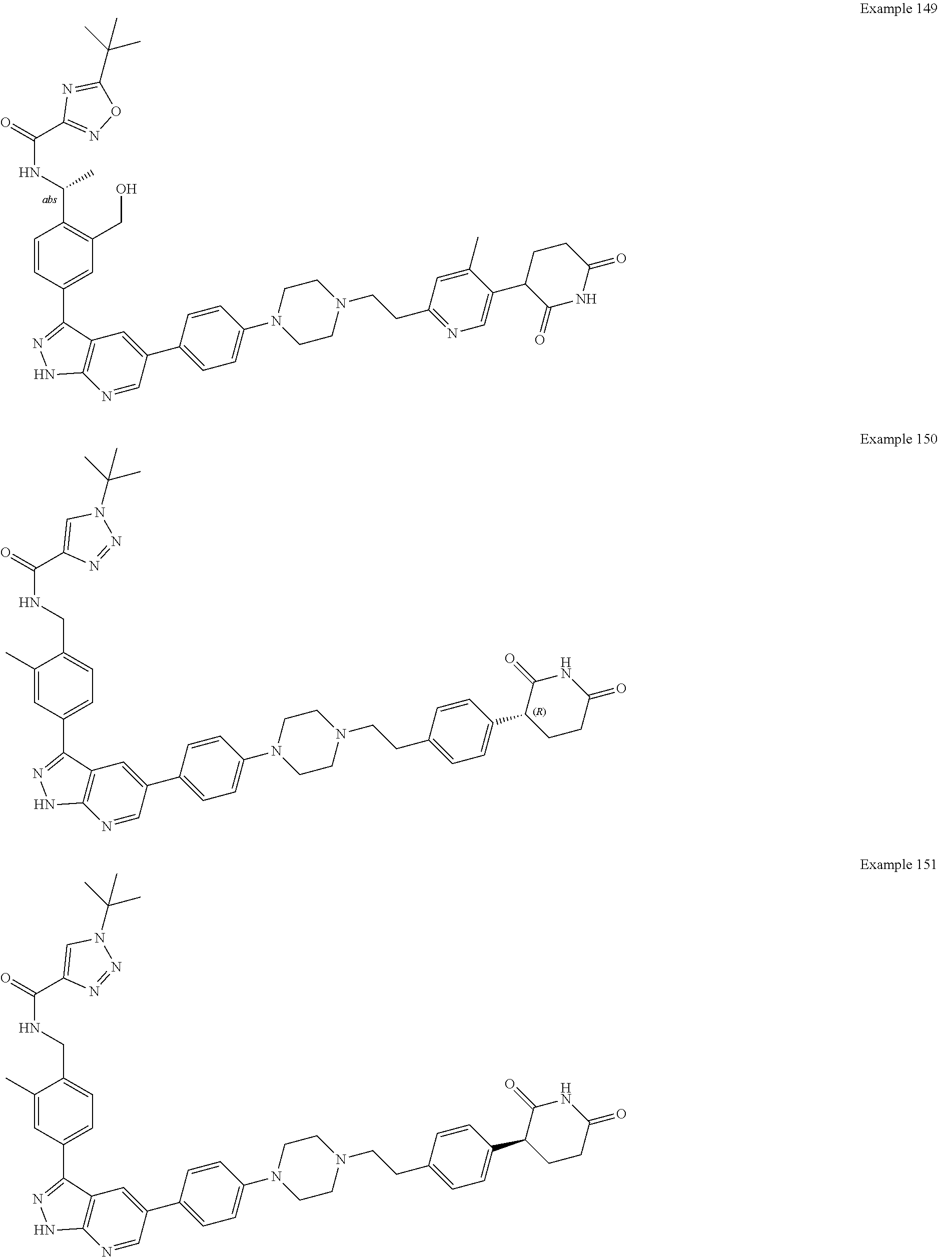

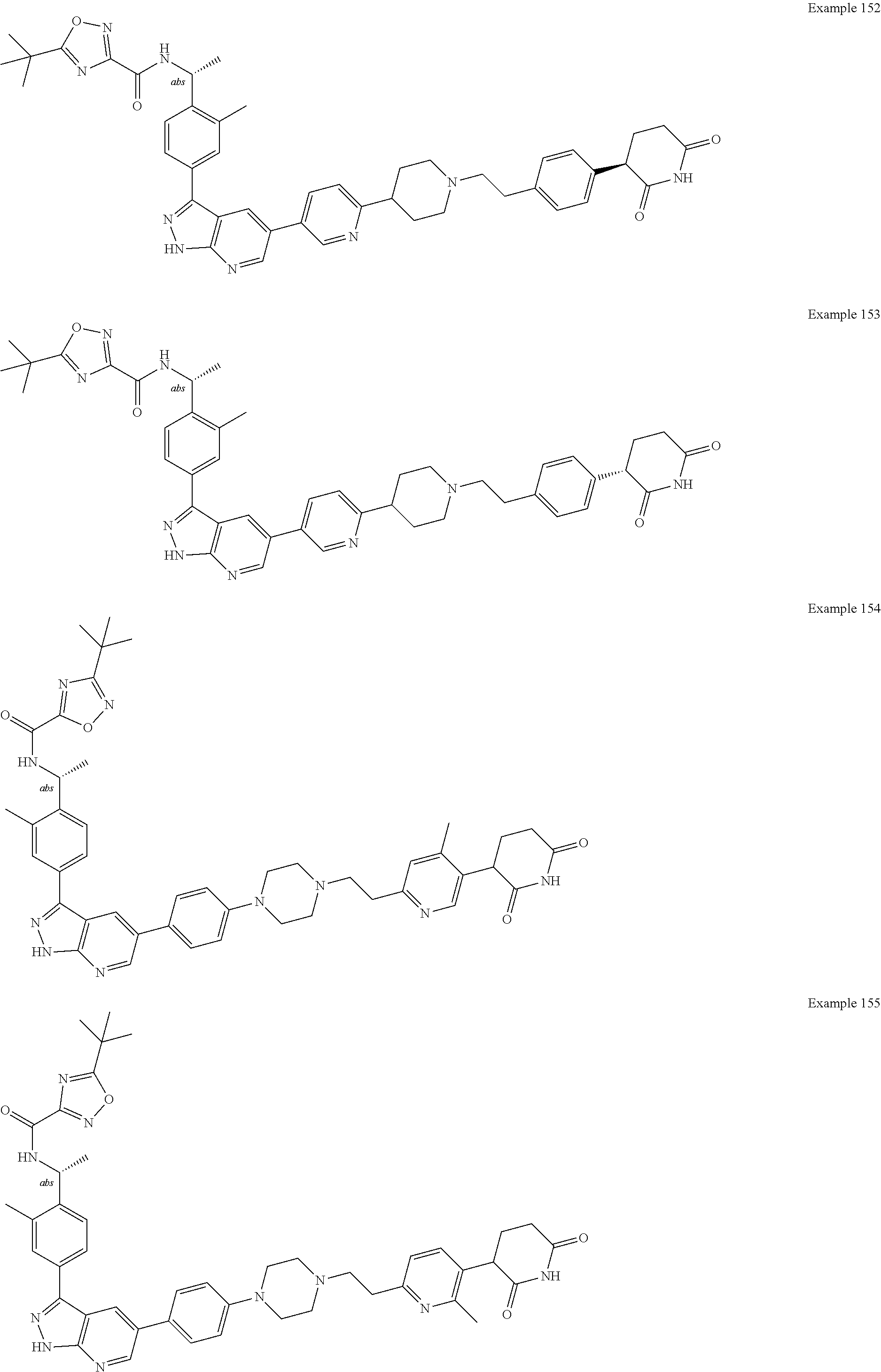

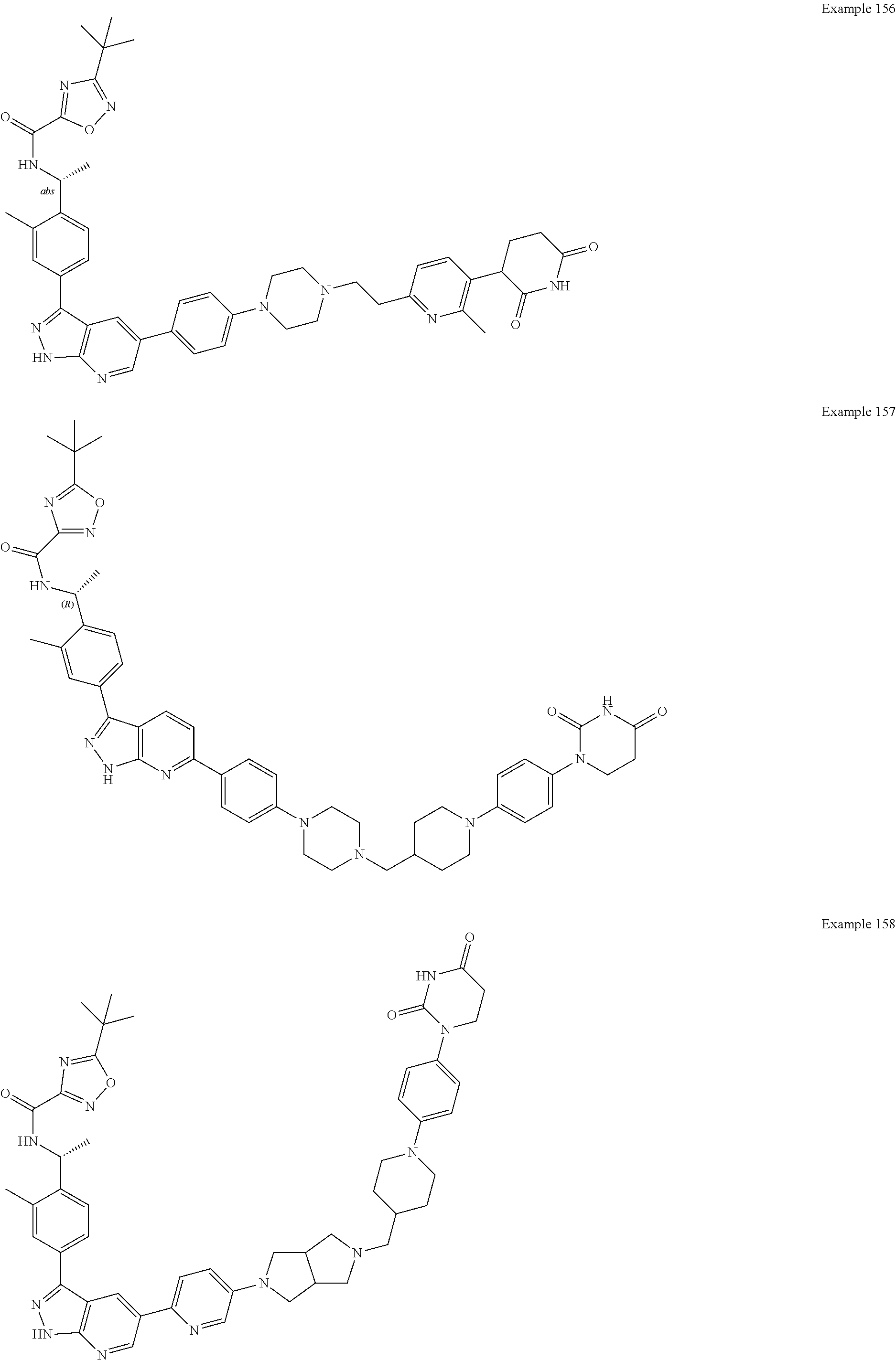

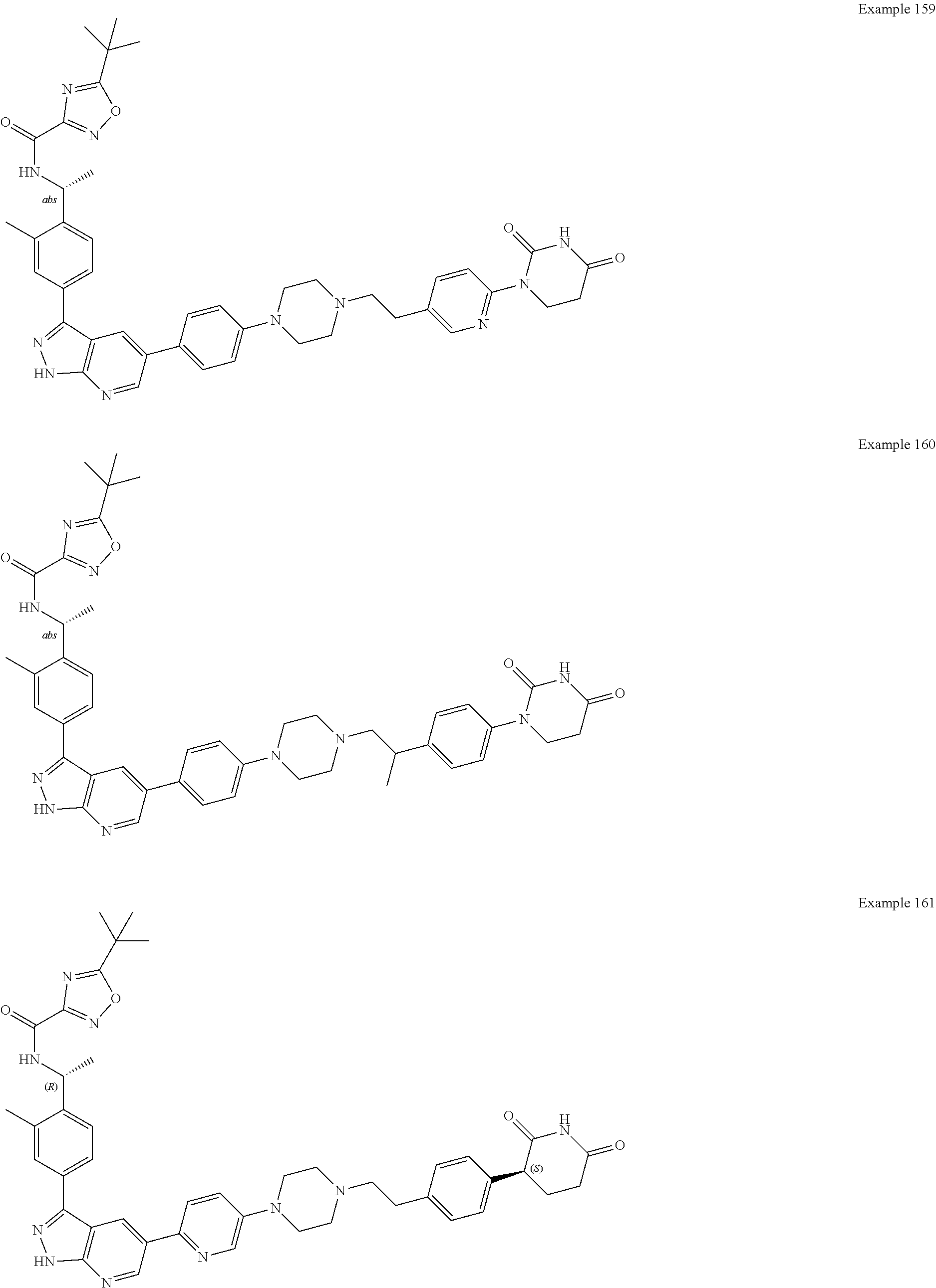

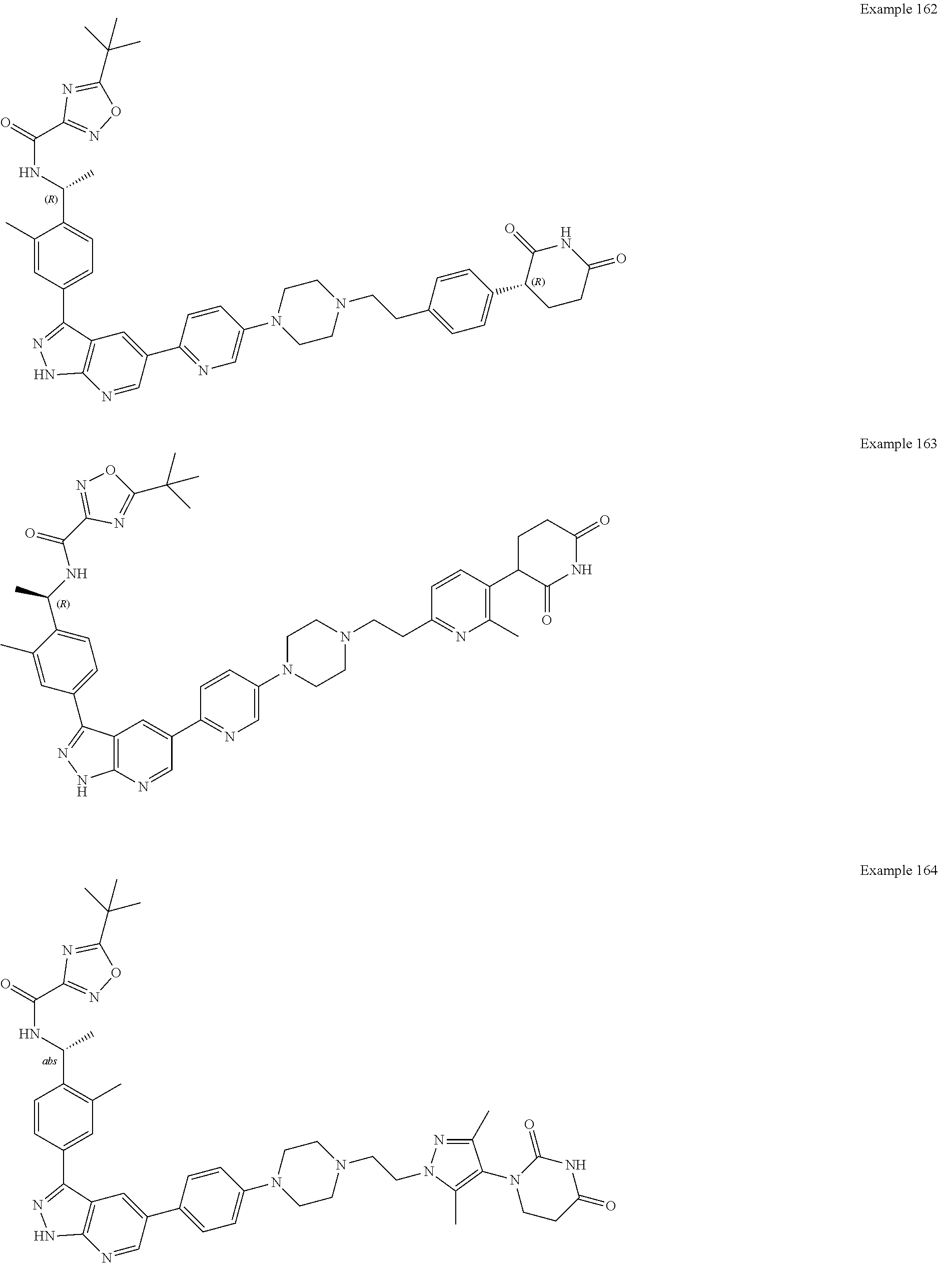

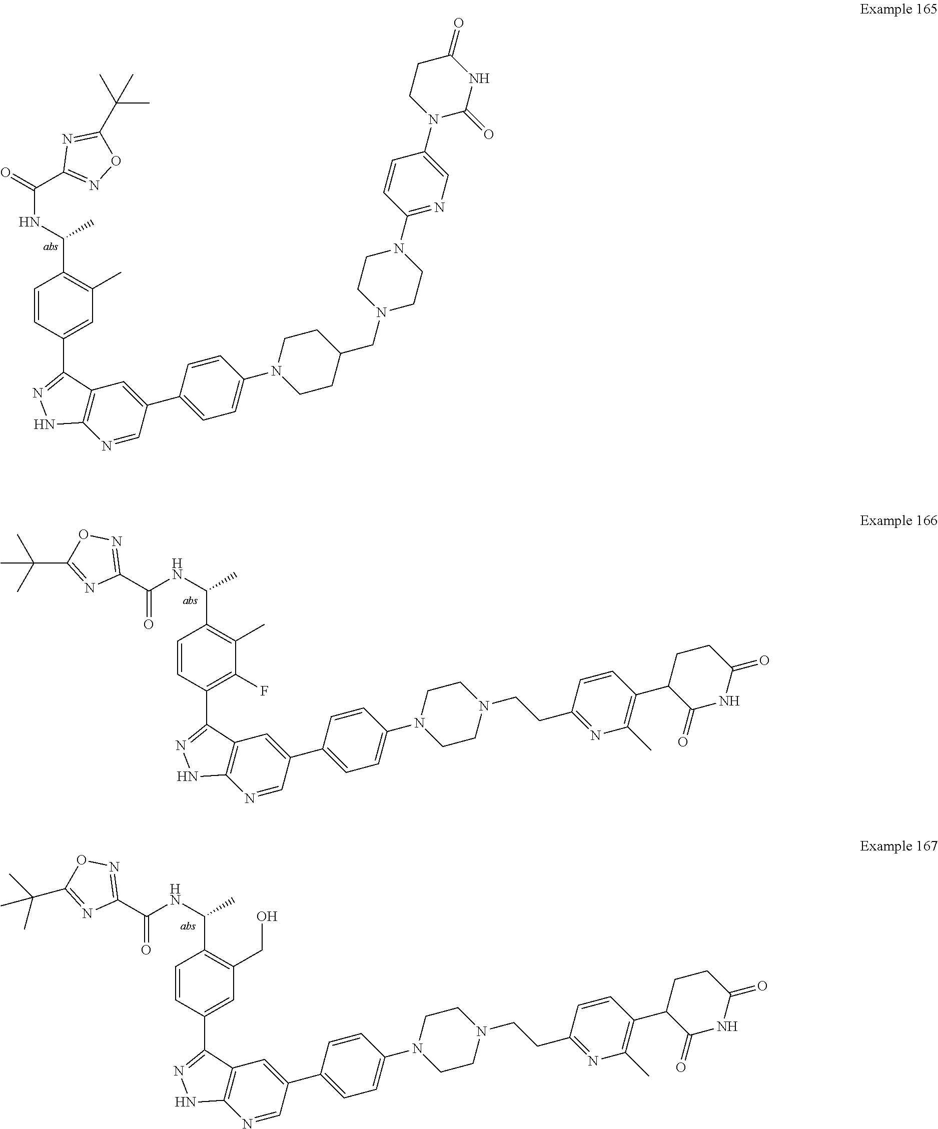 [SMILES: Cc1cc(-c2n[nH]c3ncc(-c4ccc(N5CCC(CN6CCN(c7ccc(N8CCC(=O)NC8=O)cn7)CC6)CC5)cc4)cc23)ccc1[C@@H](C)NC(=O)c1noc(C(C)(C)C)n1 ; Cc1nc(CCN2CCN(c3ccc(-c4cnc5[nH]nc(-c6ccc([C@@H](C)NC(=O)c7noc(C(C)(C)C)n7)c(C)c6F)c5c4)cc3)CC2)ccc1C1CCC(=O)NC1=O ; Cc1nc(CCN2CCN(c3ccc(-c4cnc5[nH]nc(-c6ccc([C@@H](C)NC(=O)c7noc(C(C)(C)C)n7)c(CO)c6)c5c4)cc3)CC2)ccc1C1CCC(=O)NC1=O]

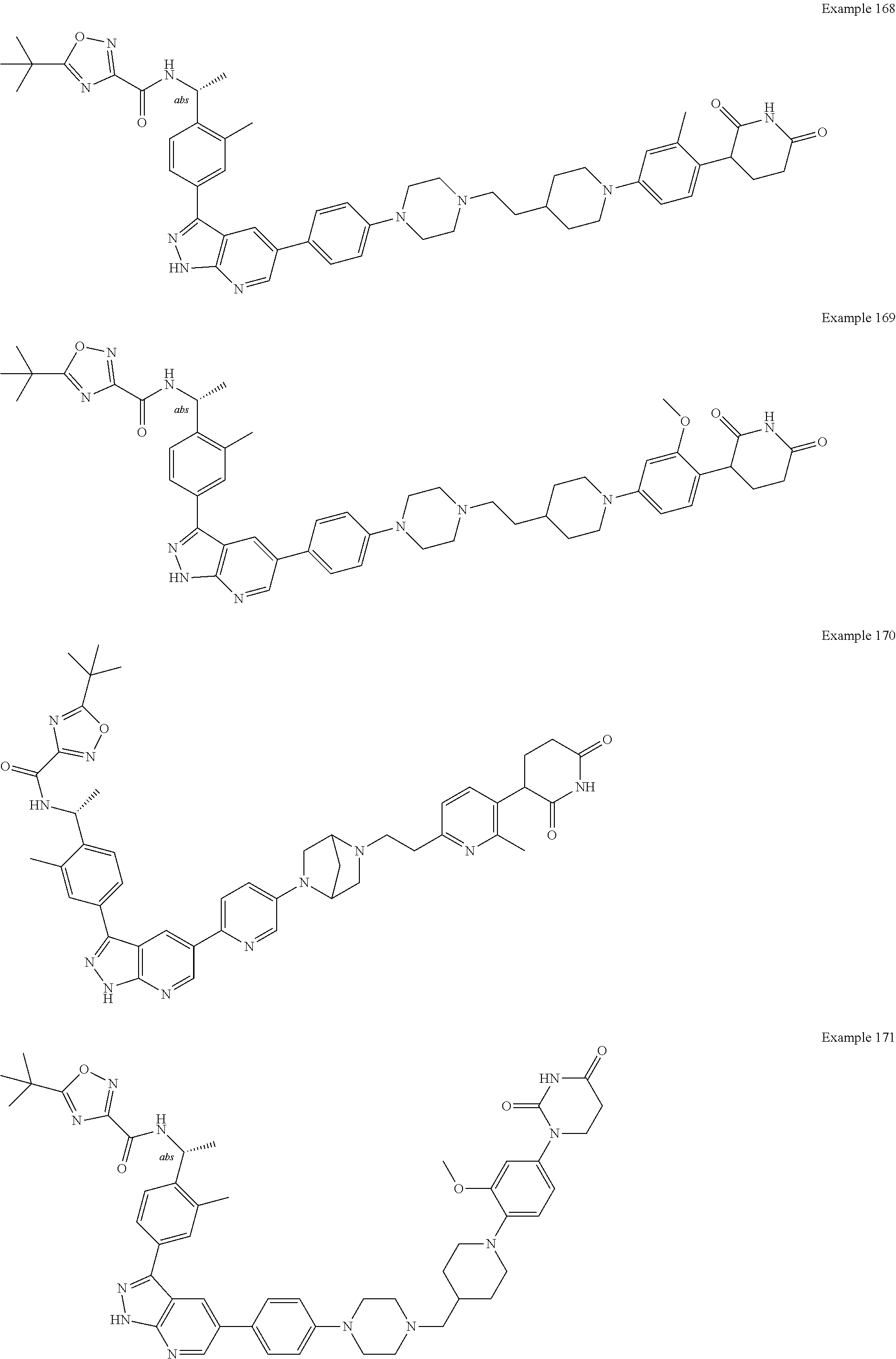 [SMILES: Cc1cc(N2CCC(CCN3CCN(c4ccc(-c5cnc6[nH]nc(-c7ccc([C@@H](C)NC(=O)c8noc(C(C)(C)C)n8)c(C)c7)c6c5)cc4)CC3)CC2)ccc1C1CCC(=O)NC1=O ; COc1cc(N2CCC(CCN3CCN(c4ccc(-c5cnc6[nH]nc(-c7ccc([C@@H](C)NC(=O)c8noc(C(C)(C)C)n8)c(C)c7)c6c5)cc4)CC3)CC2)ccc1C1CCC(=O)NC1=O ; Cc1cc(-c2n[nH]c3ncc(-c4ccc(N5CC6CC5CN6CCc5ccc(C6CCC(=O)NC6=O)c(C)n5)cn4)cc23)ccc1[C@@H](C)NC(=O)c1noc(C(C)(C)C)n1 ; COc1cc(N2CCC(=O)NC2=O)ccc1N1CCC(CN2CCN(c3ccc(-c4cnc5[nH]nc(-c6ccc([C@@H](C)NC(=O)c7noc(C(C)(C)C)n7)c(C)c6)c5c4)cc3)CC2)CC1]

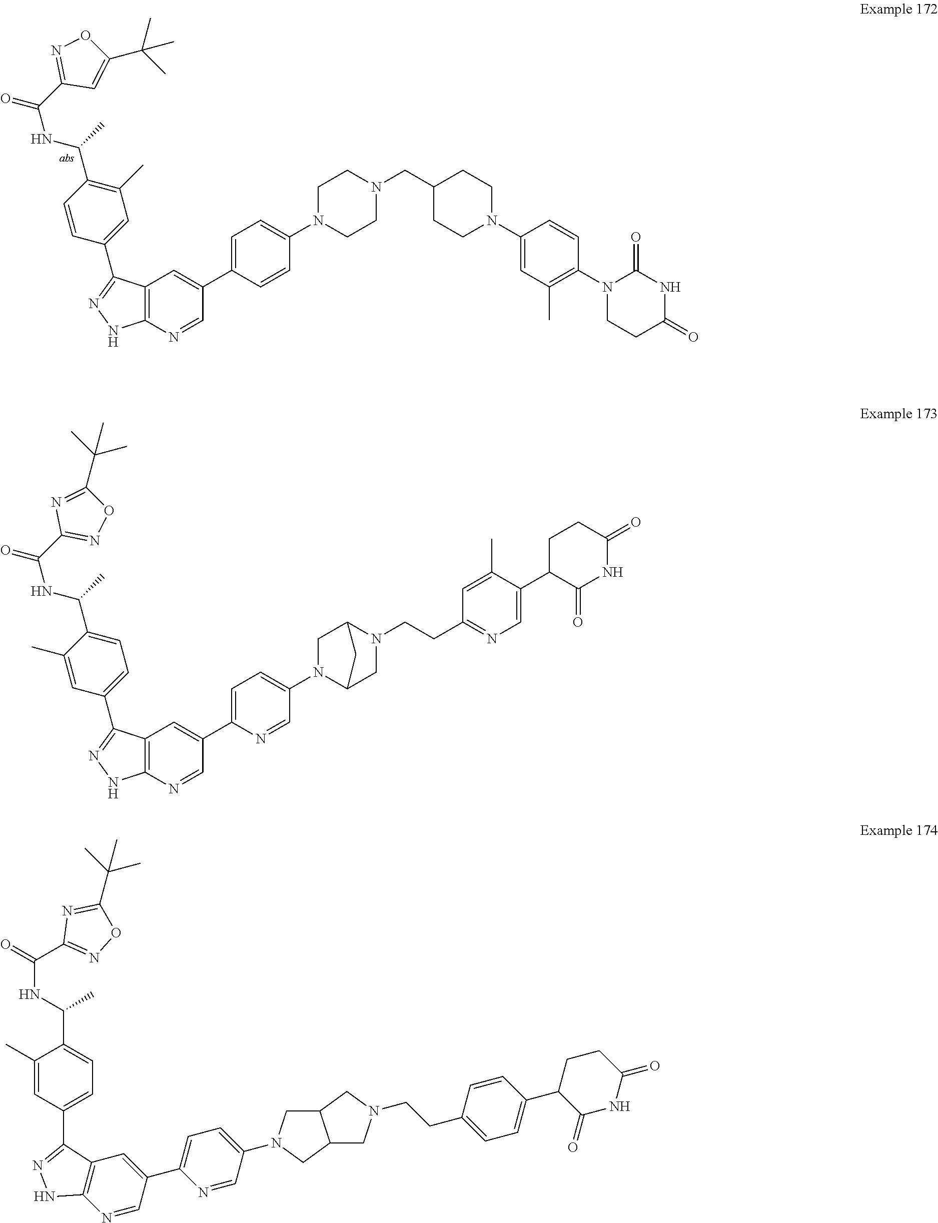 [SMILES: Cc1cc(-c2n[nH]c3ncc(-c4ccc(N5CCN(CC6CCN(c7ccc(N8CCC(=O)NC8=O)c(C)c7)CC6)CC5)cc4)cc23)ccc1[C@@H](C)NC(=O)c1cc(C(C)(C)C)on1 ; Cc1cc(CCN2CC3CC2CN3c2ccc(-c3cnc4[nH]nc(-c5ccc([C@@H](C)NC(=O)c6noc(C(C)(C)C)n6)c(C)c5)c4c3)nc2)ncc1C1CCC(=O)NC1=O ; Cc1cc(-c2n[nH]c3ncc(-c4ccc(N5CC6CN(CCc7ccc(C8CCC(=O)NC8=O)cc7)CC6C5)cn4)cc23)ccc1[C@@H](C)NC(=O)c1noc(C(C)(C)C)n1]

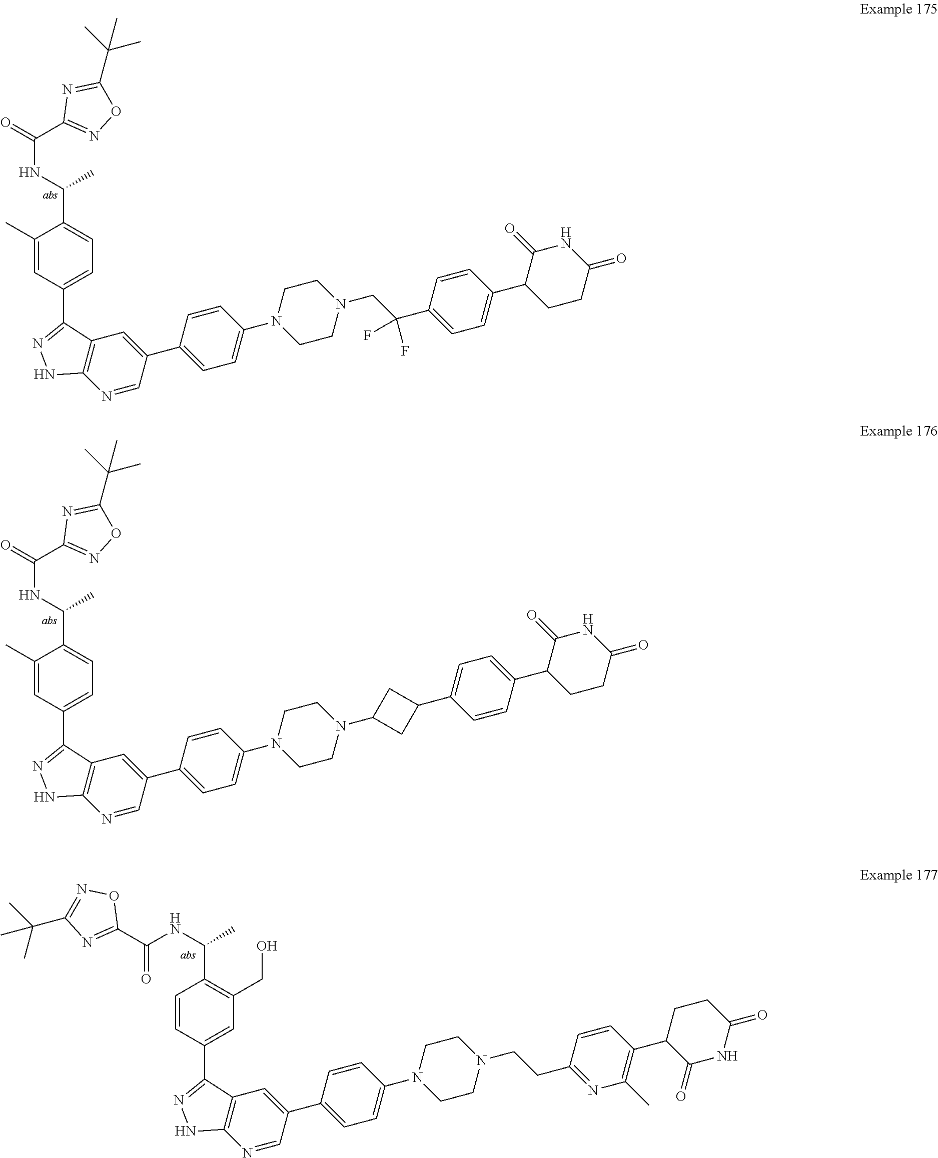

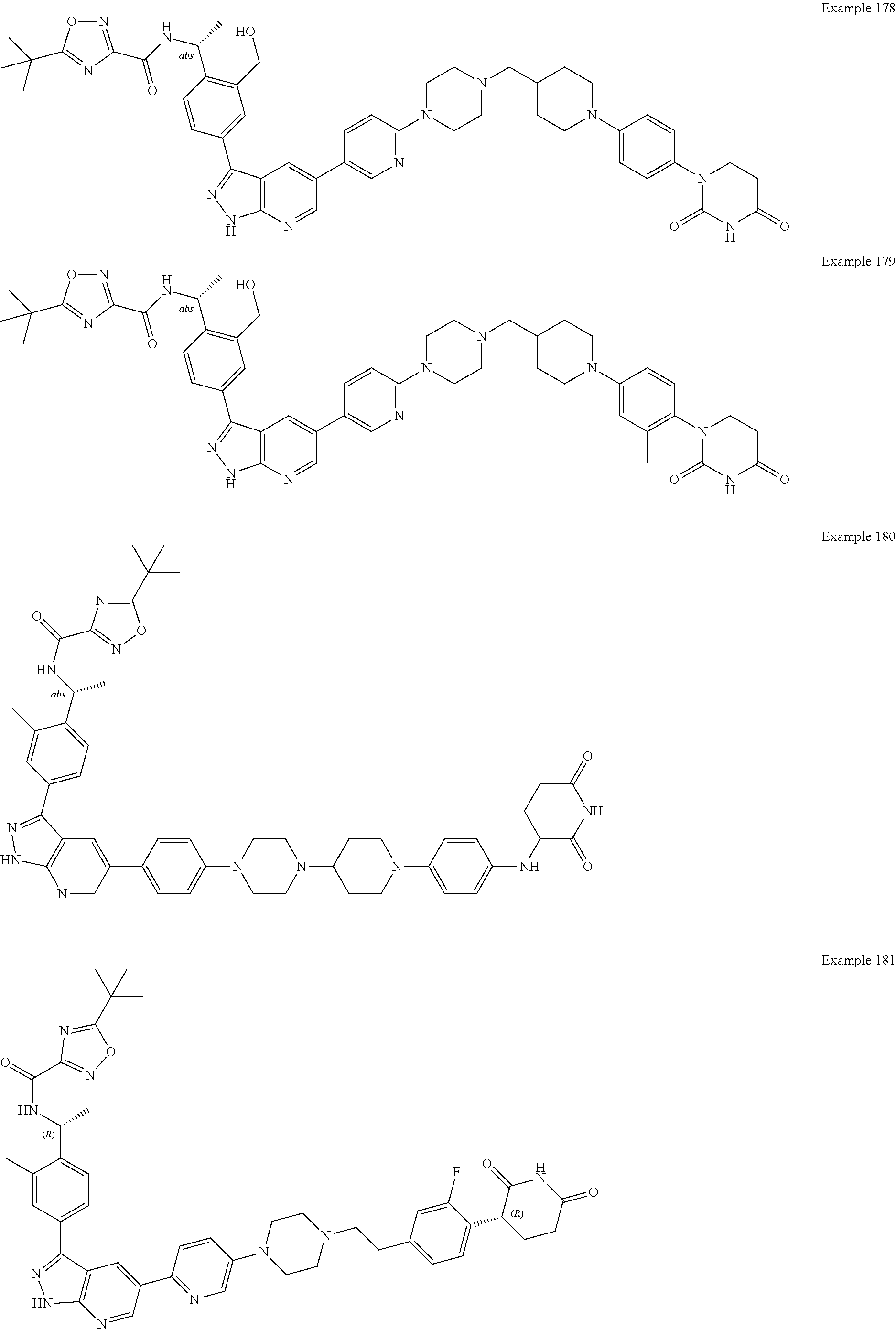

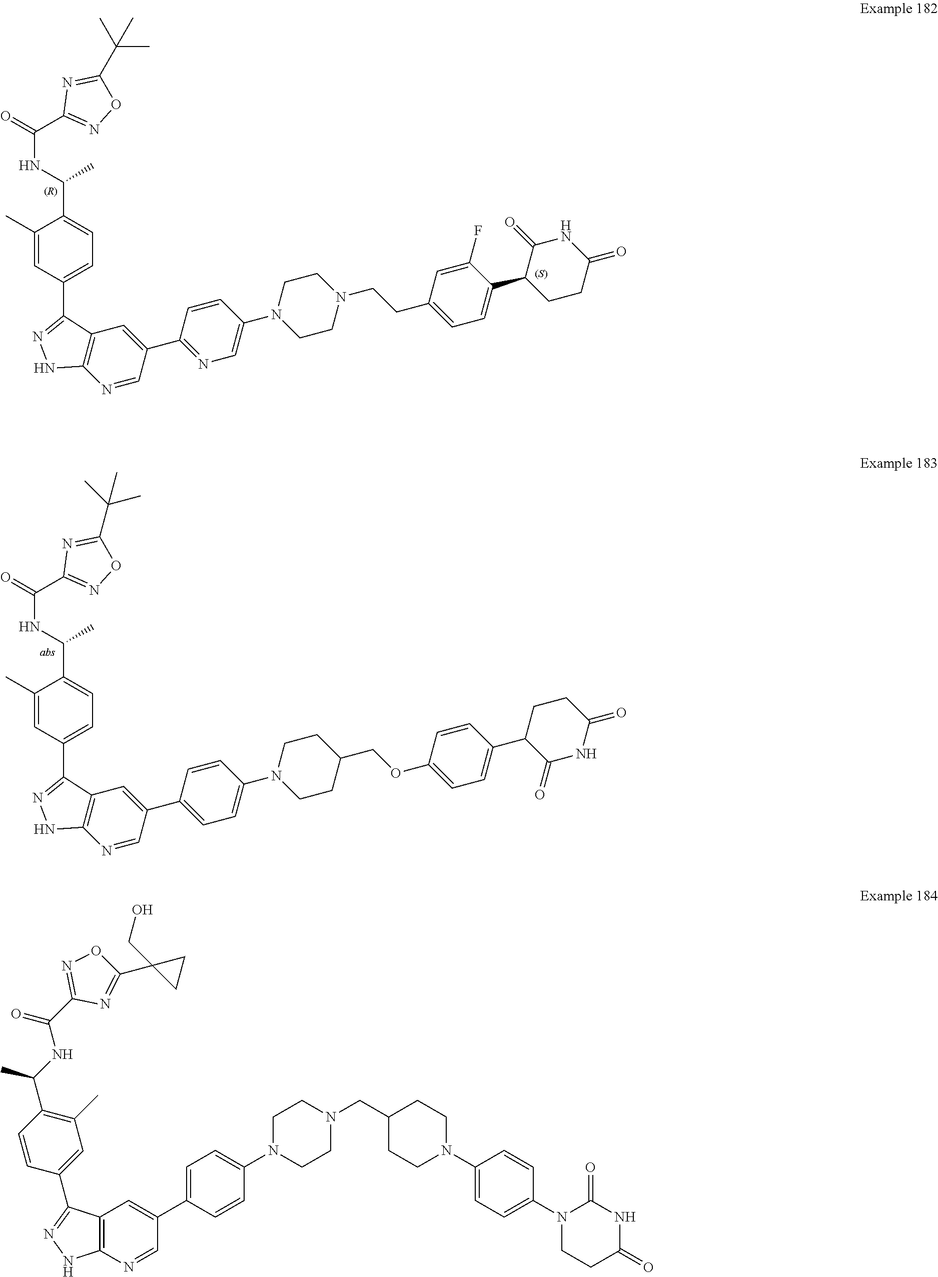

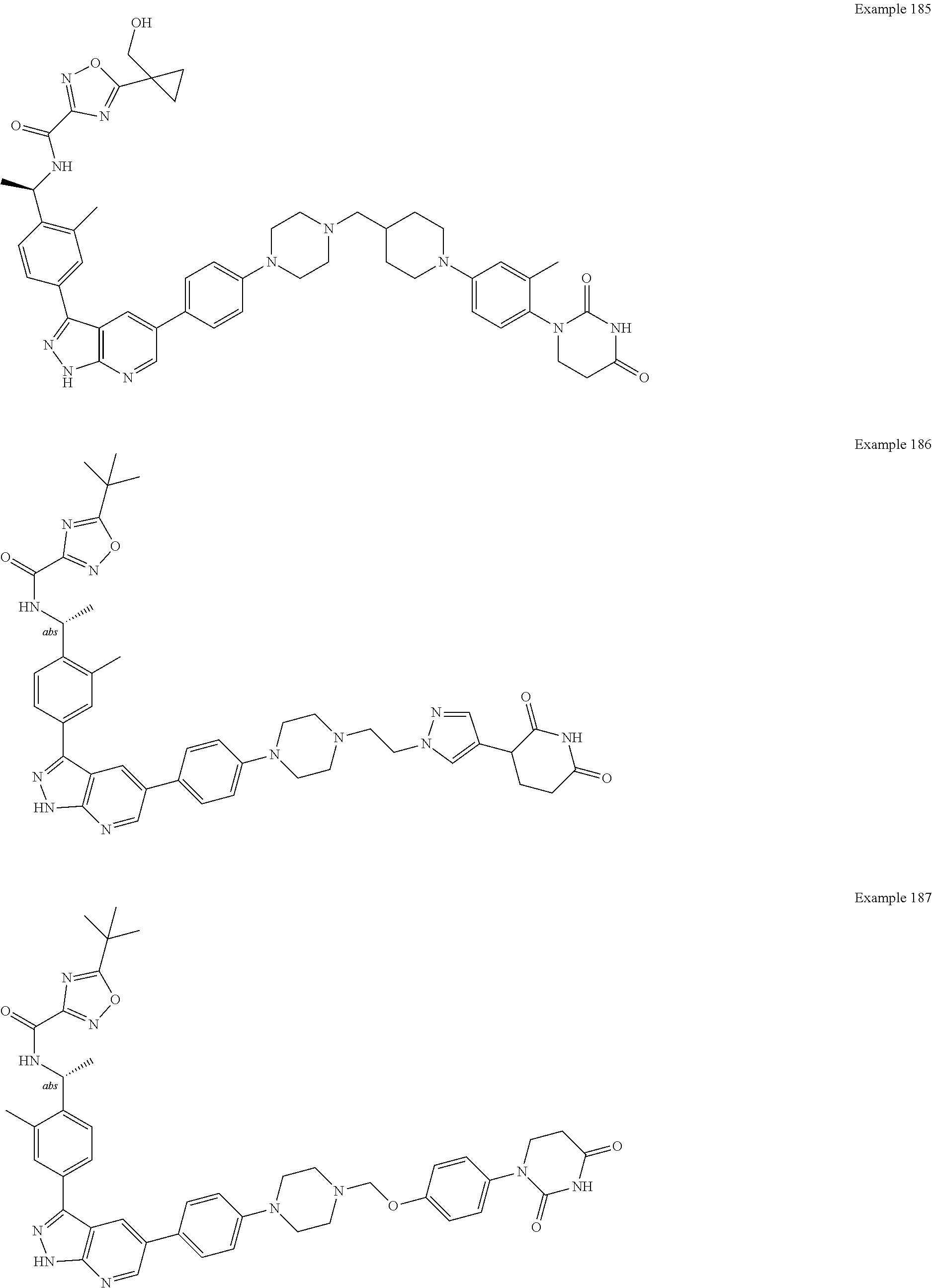

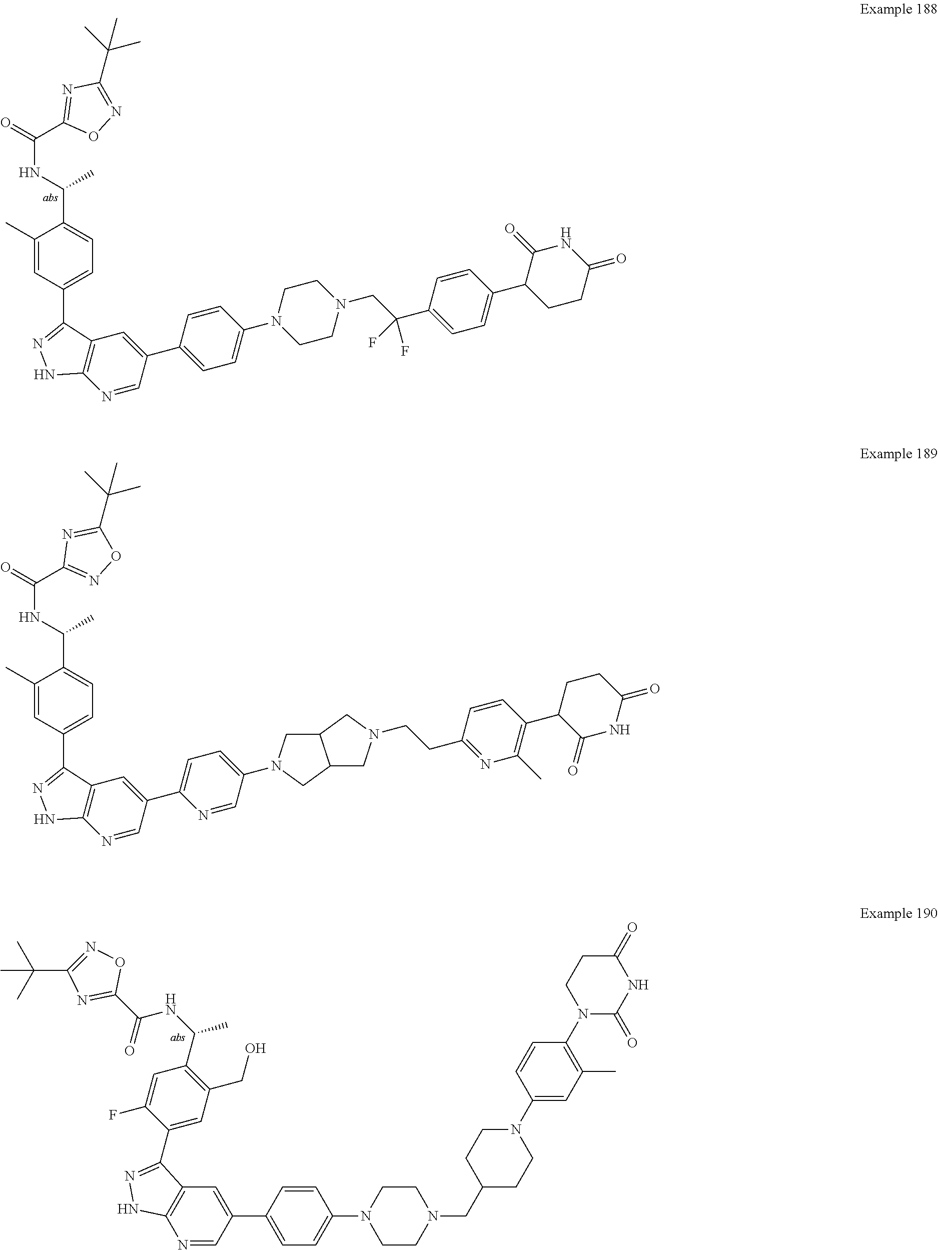

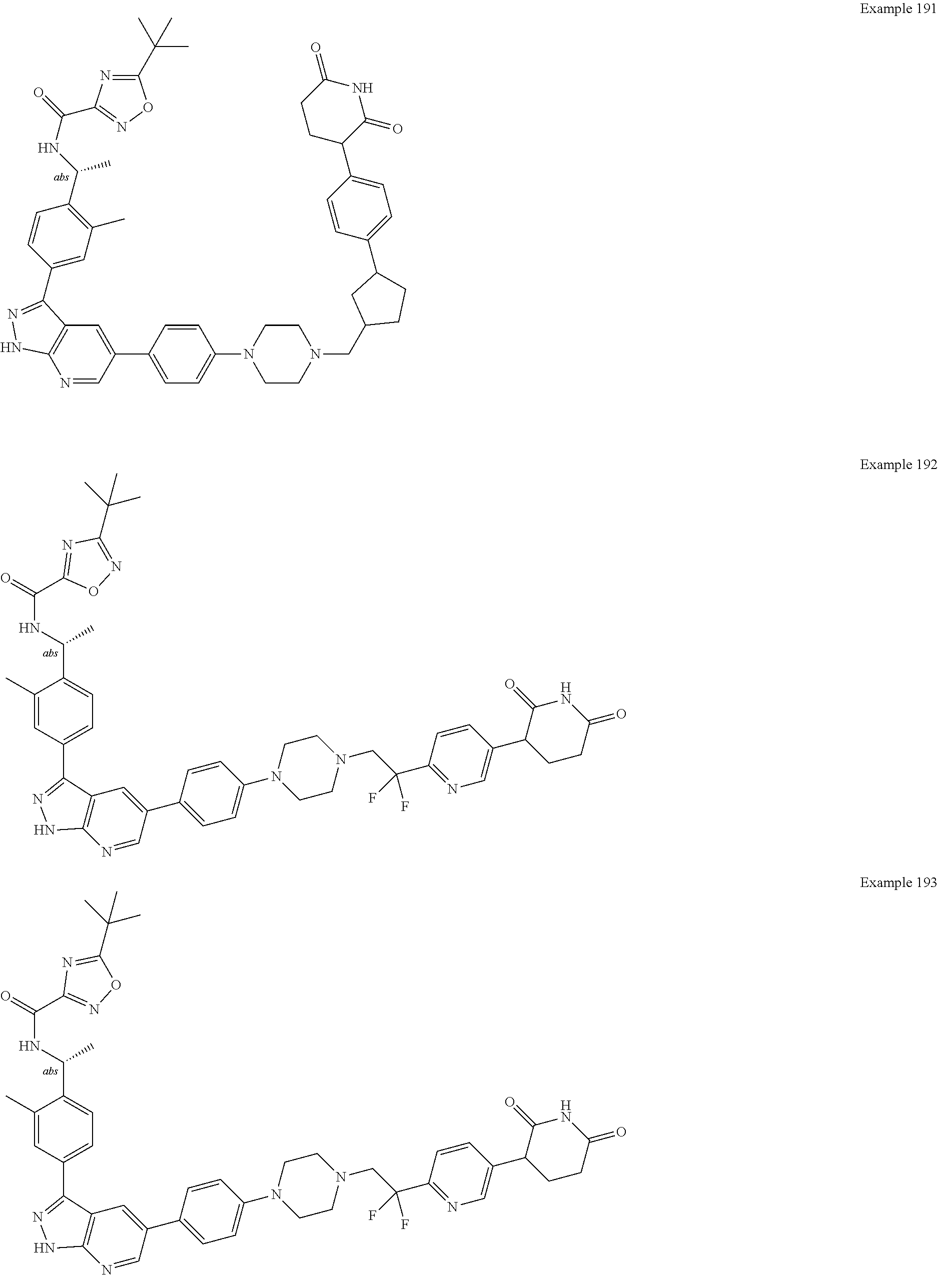

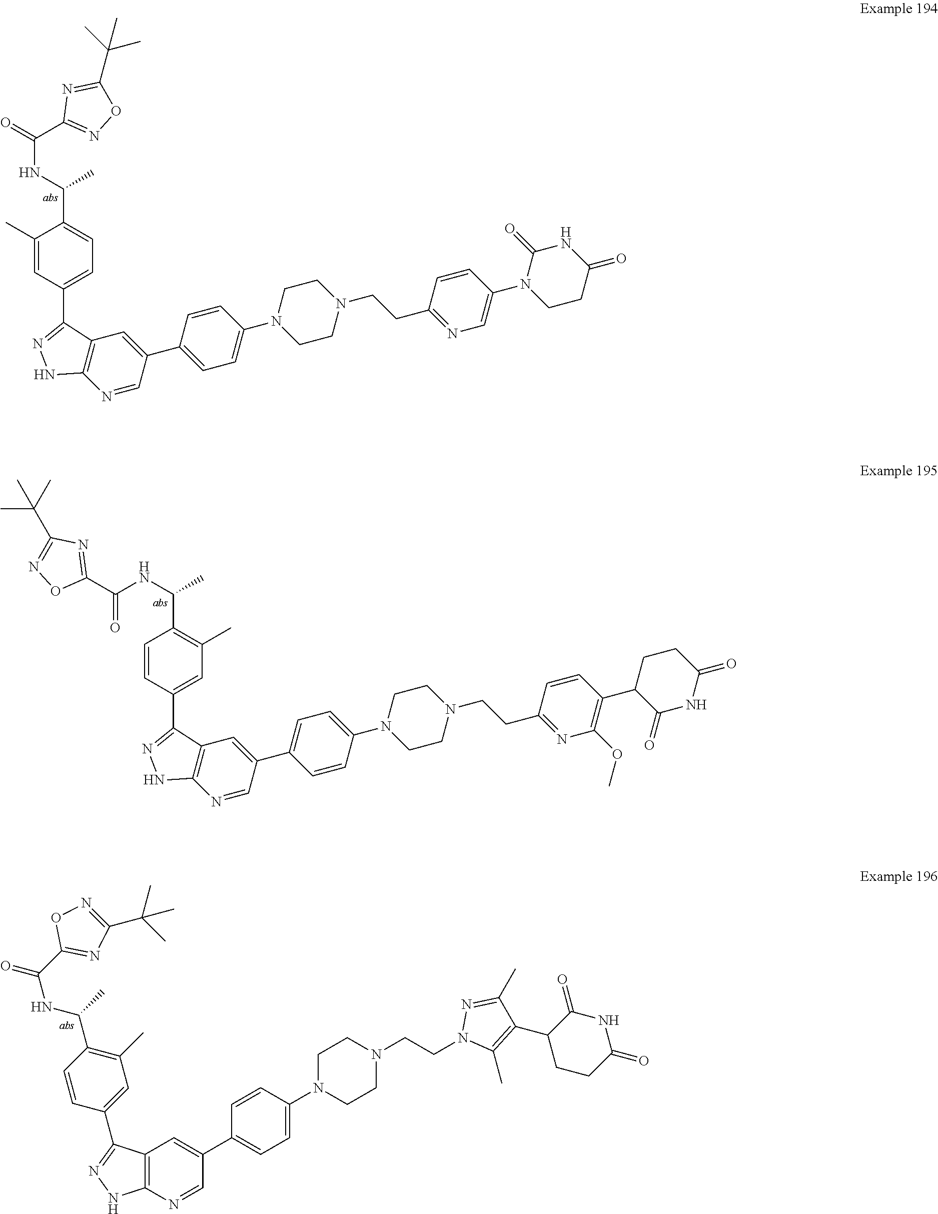

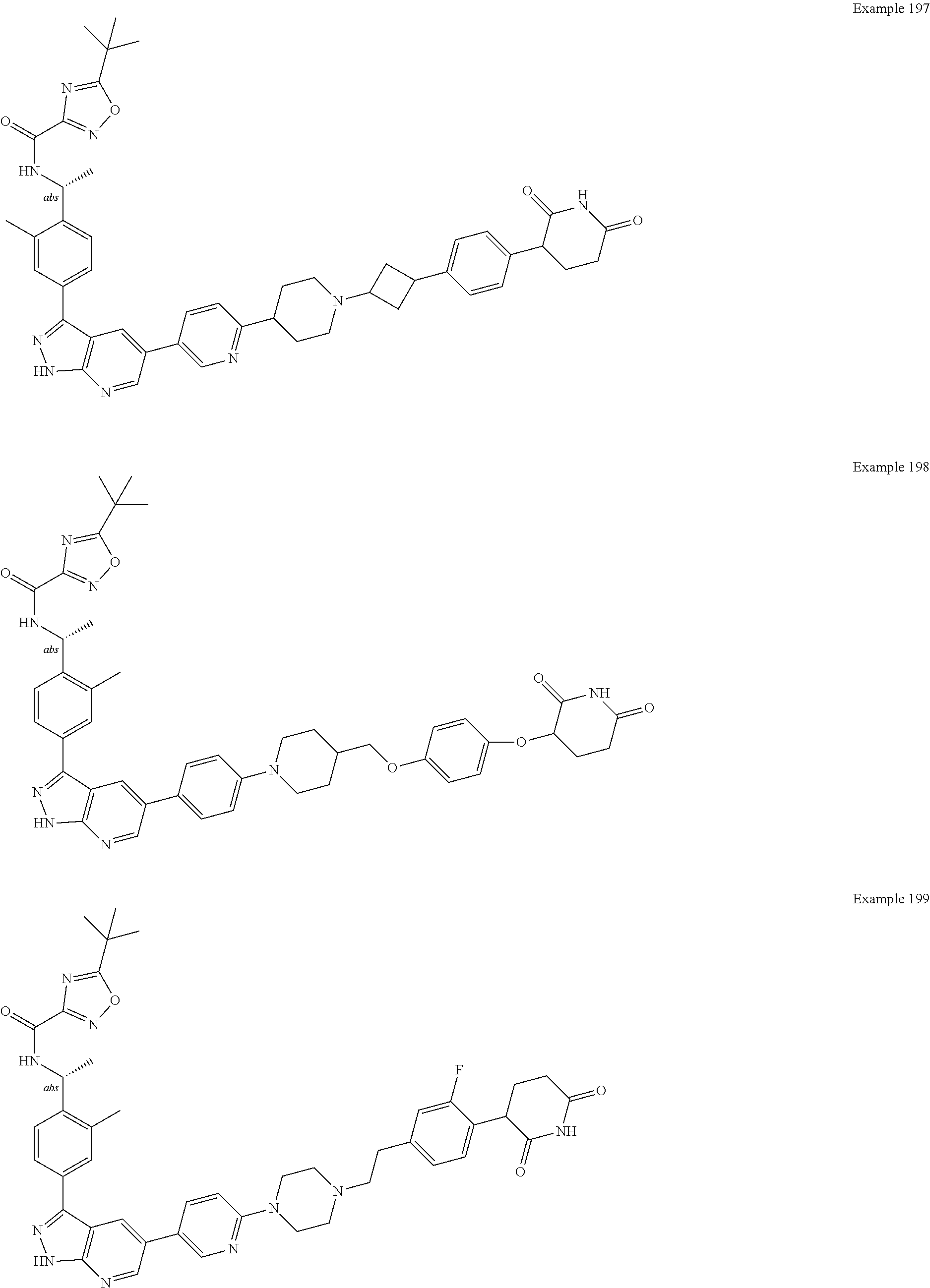

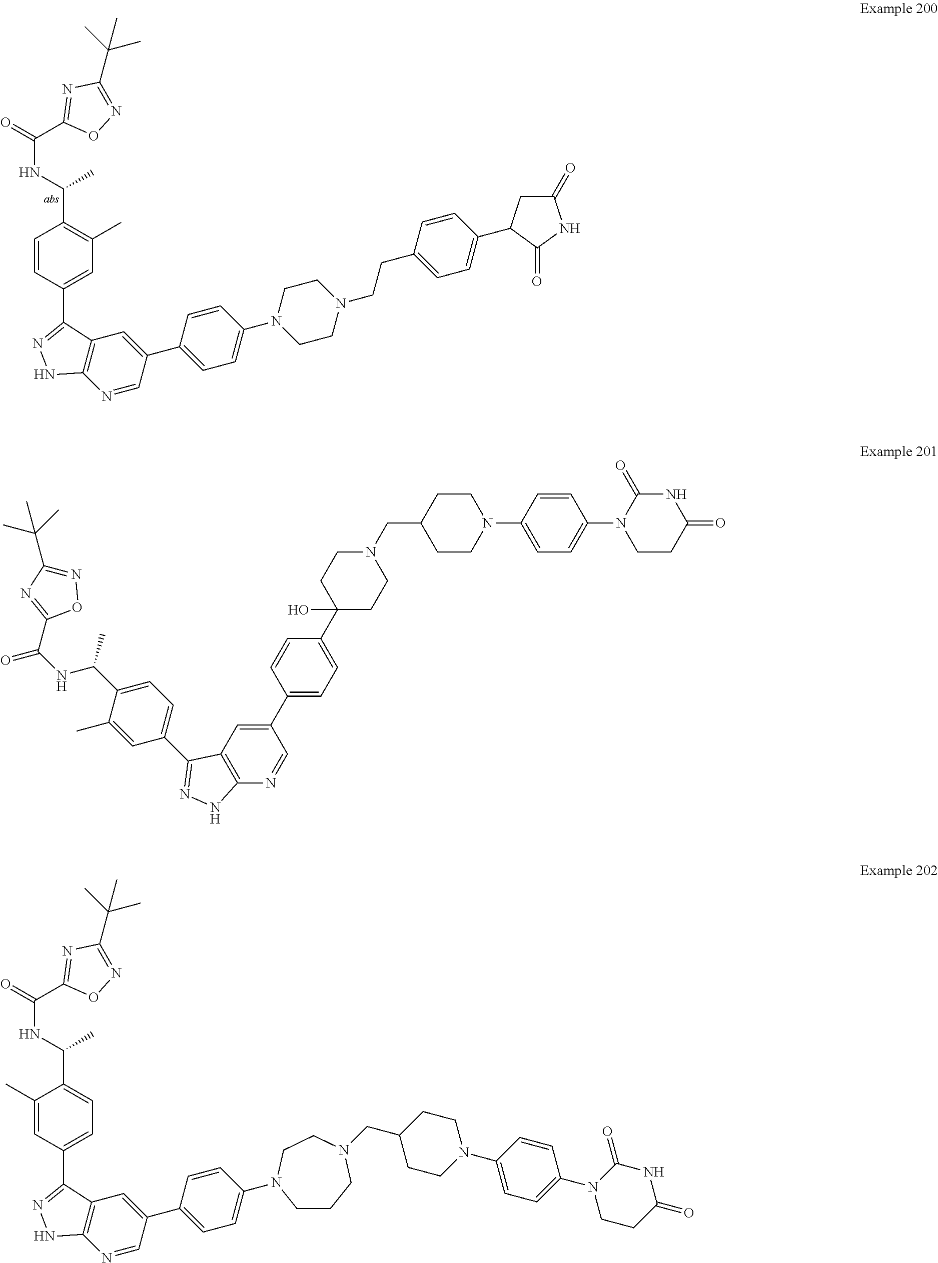

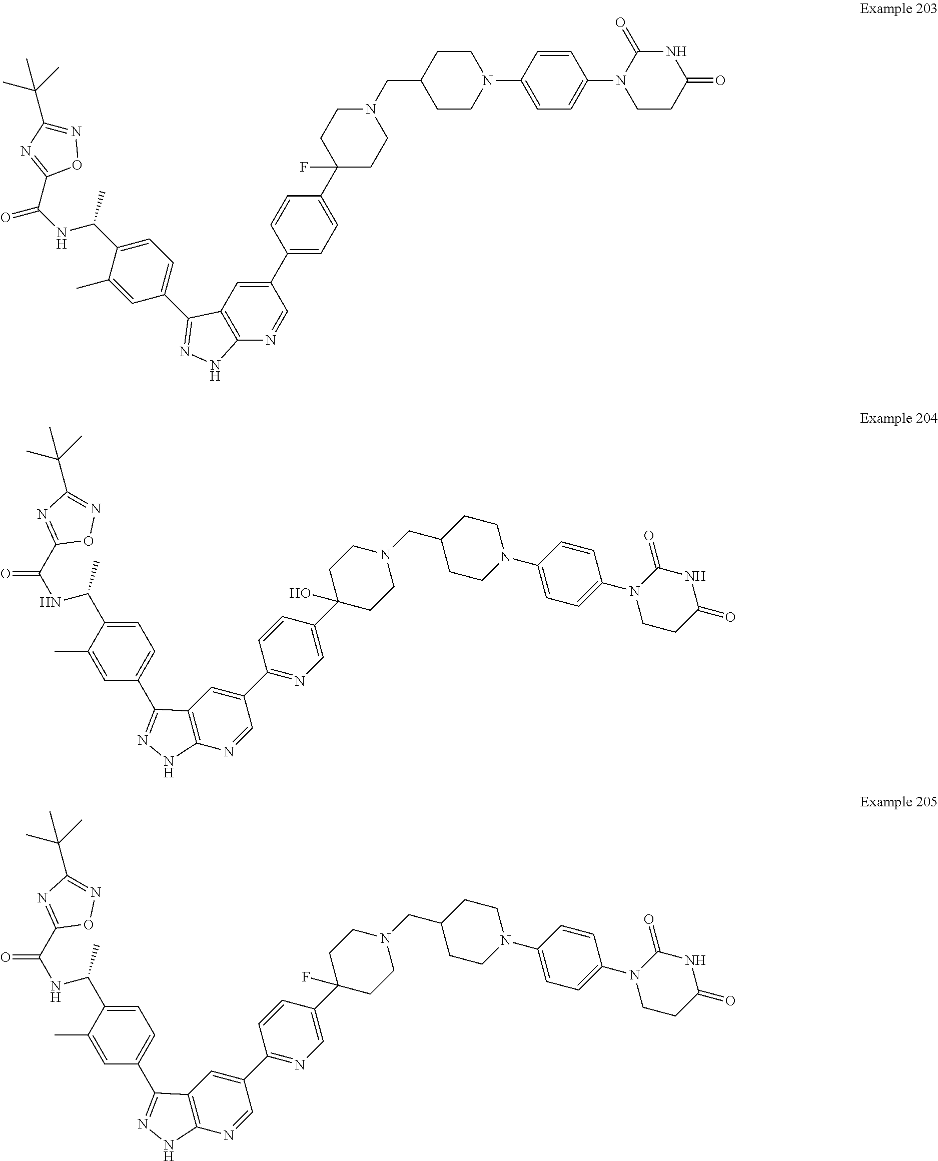 [SMILES: Cc1cc(-c2n[nH]c3ncc(-c4ccc(C5(F)CCN(CC6CCN(c7ccc(N8CCC(=O)NC8=O)cc7)CC6)CC5)cc4)cc23)ccc1[C@@H](C)NC(=O)c1nc(C(C)(C)C)no1 ; Cc1cc(-c2n[nH]c3ncc(-c4ccc(C5(O)CCN(CC6CCN(c7ccc(N8CCC(=O)NC8=O)cc7)CC6)CC5)cn4)cc23)ccc1[C@@H](C)NC(=O)c1nc(C(C)(C)C)no1 ; Cc1cc(-c2n[nH]c3ncc(-c4ccc(C5(F)CCN(CC6CCN(c7ccc(N8CCC(=O)NC8=O)cc7)CC6)CC5)cn4)cc23)ccc1[C@@H](C)NC(=O)c1nc(C(C)(C)C)no1]

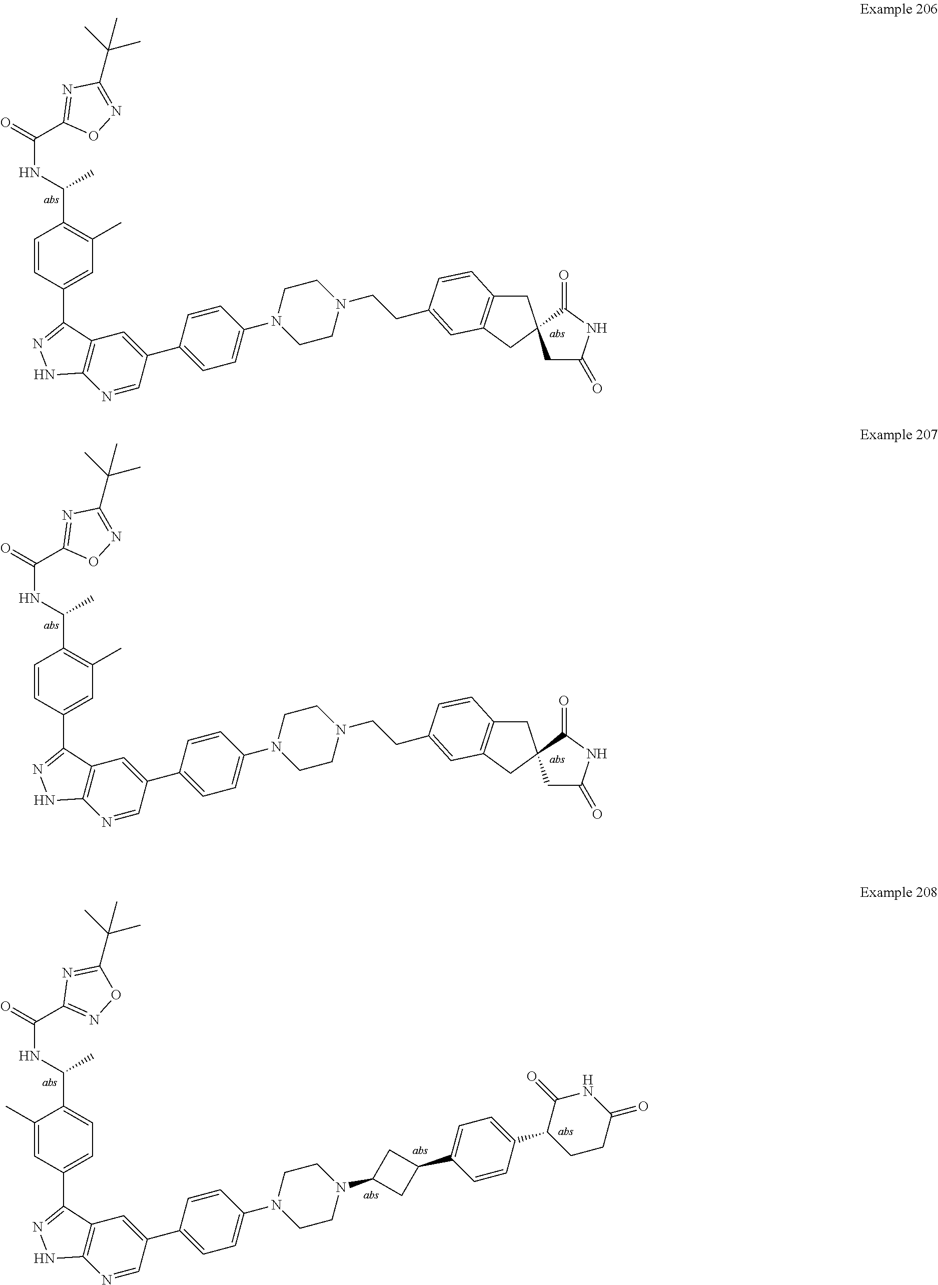

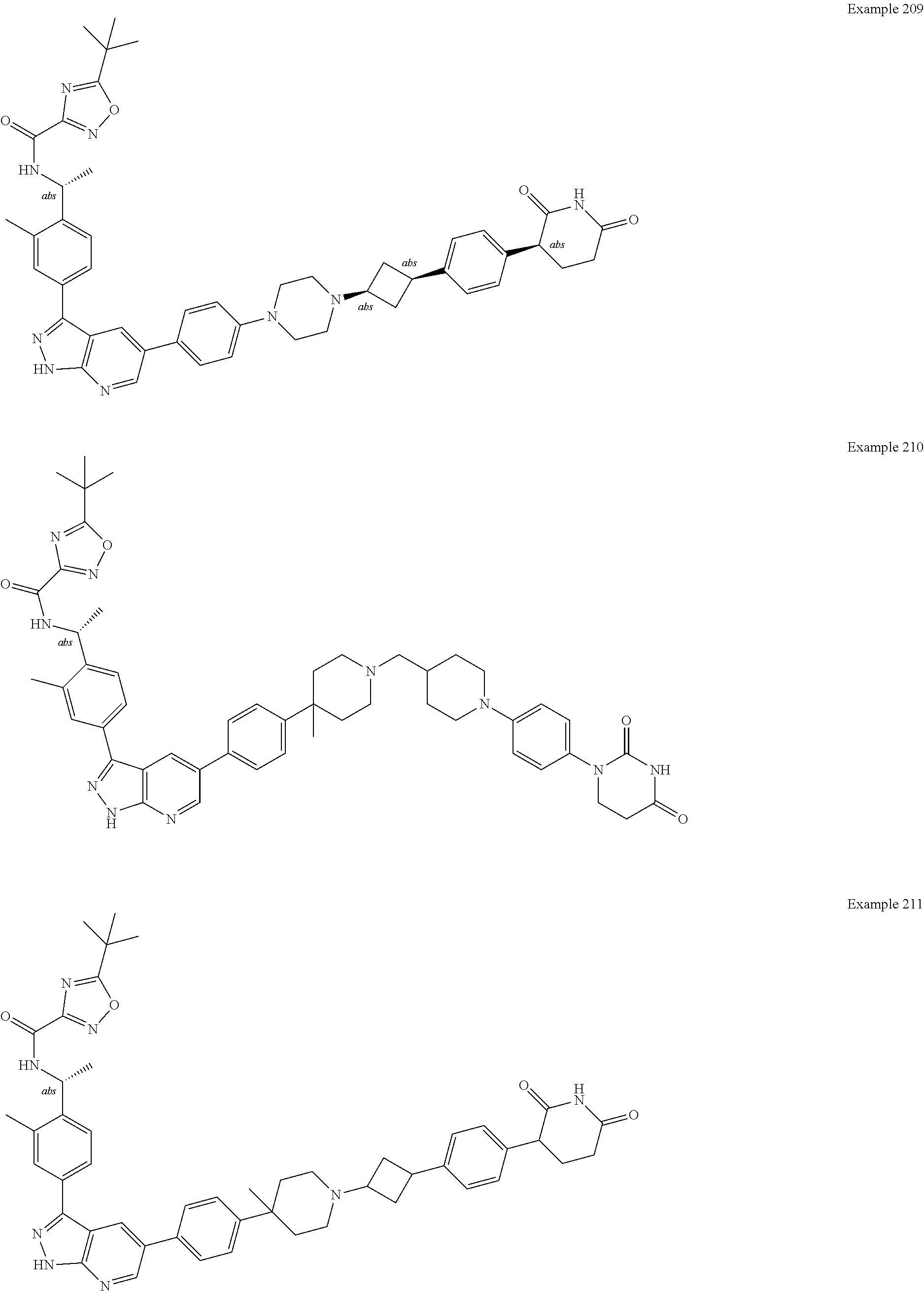

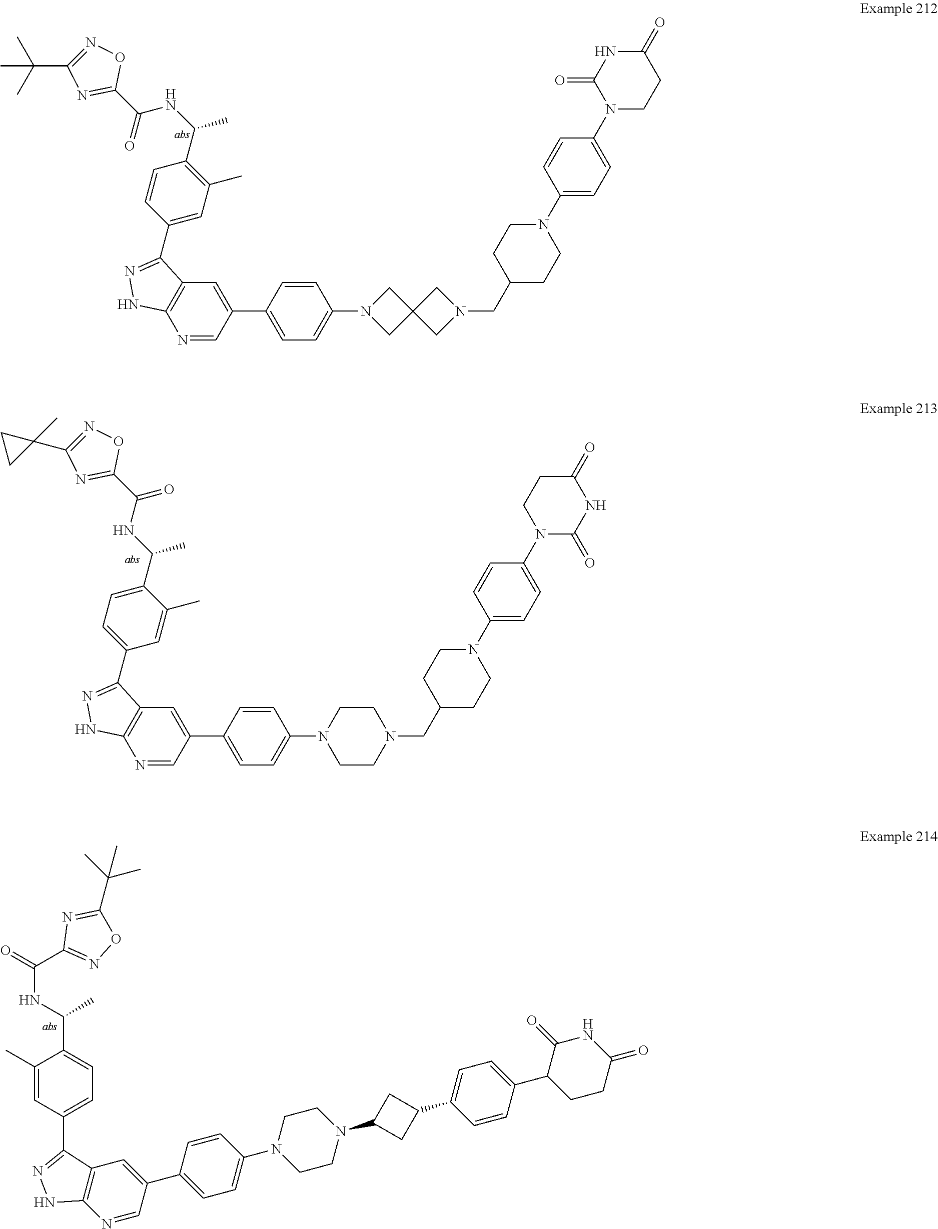

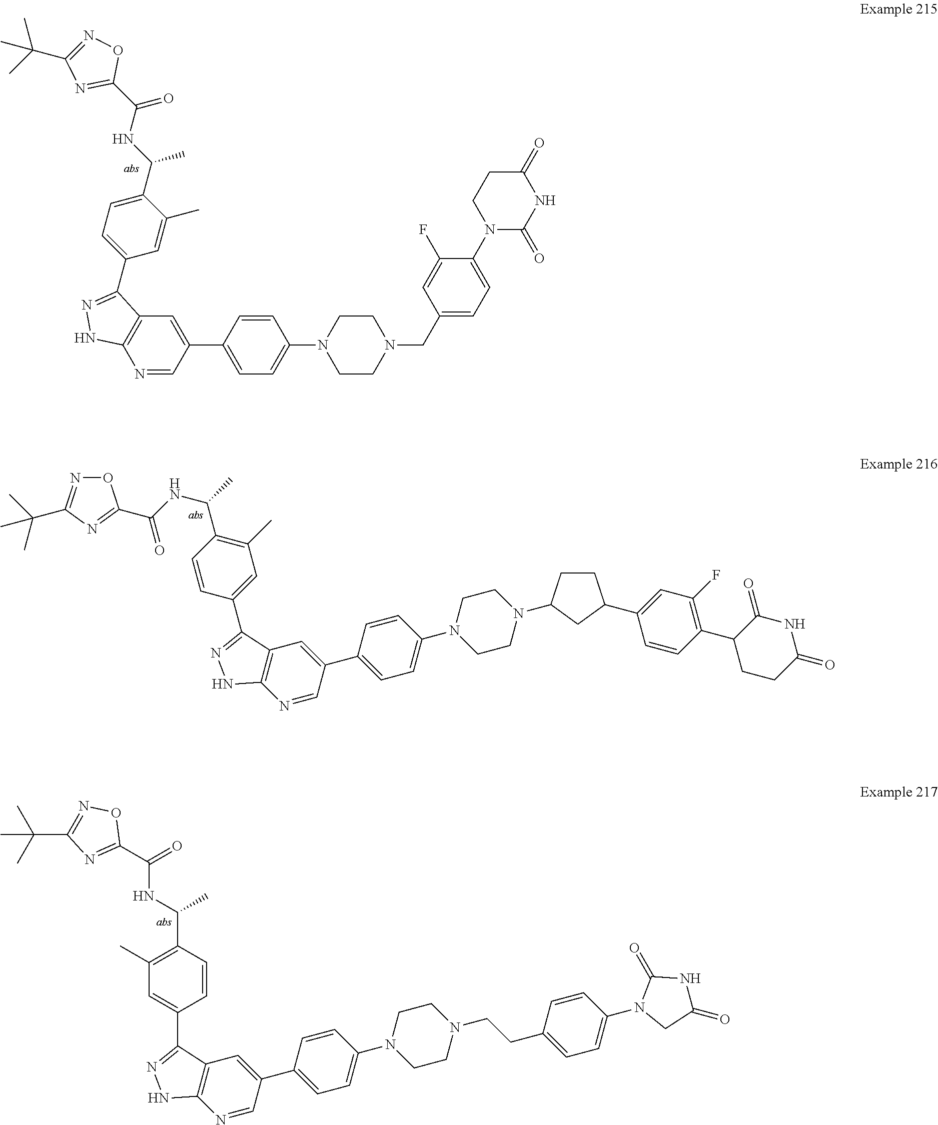

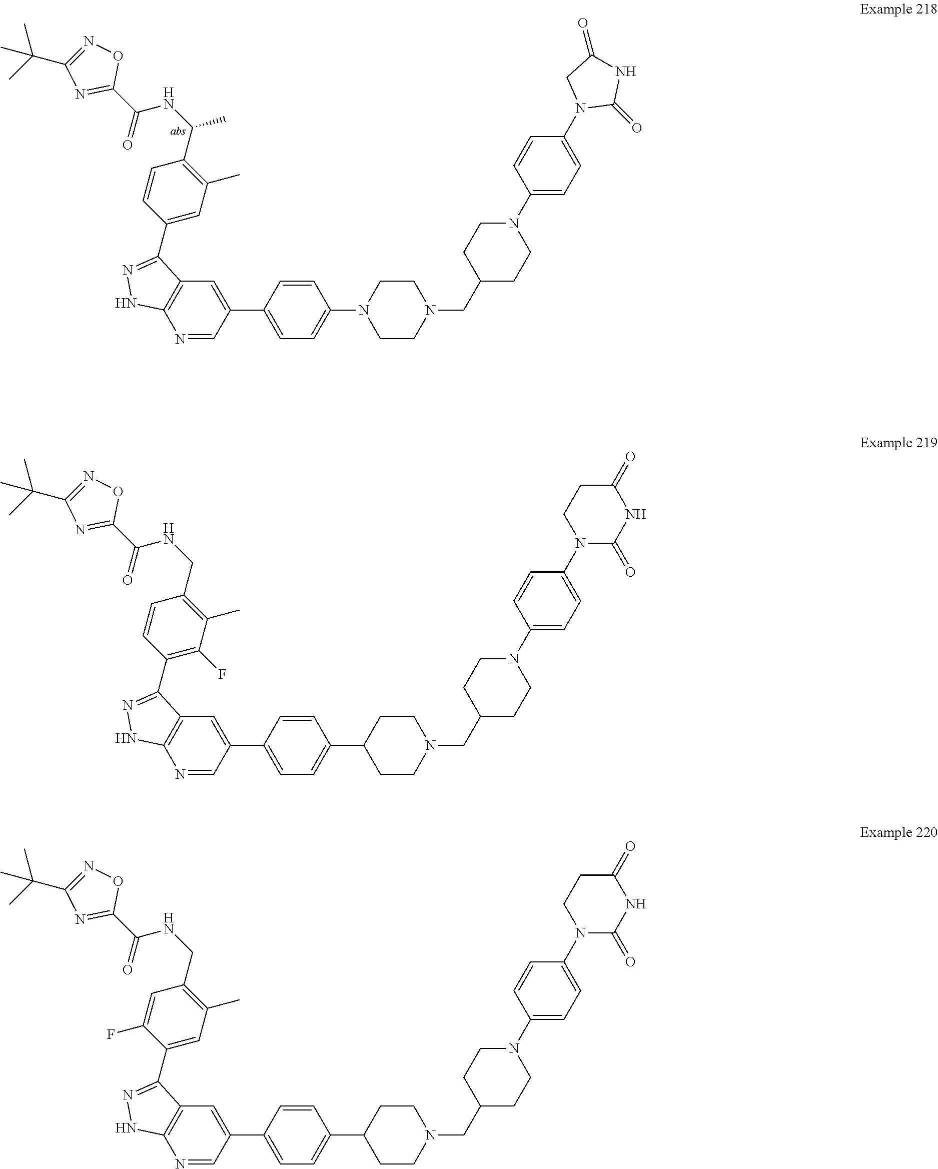

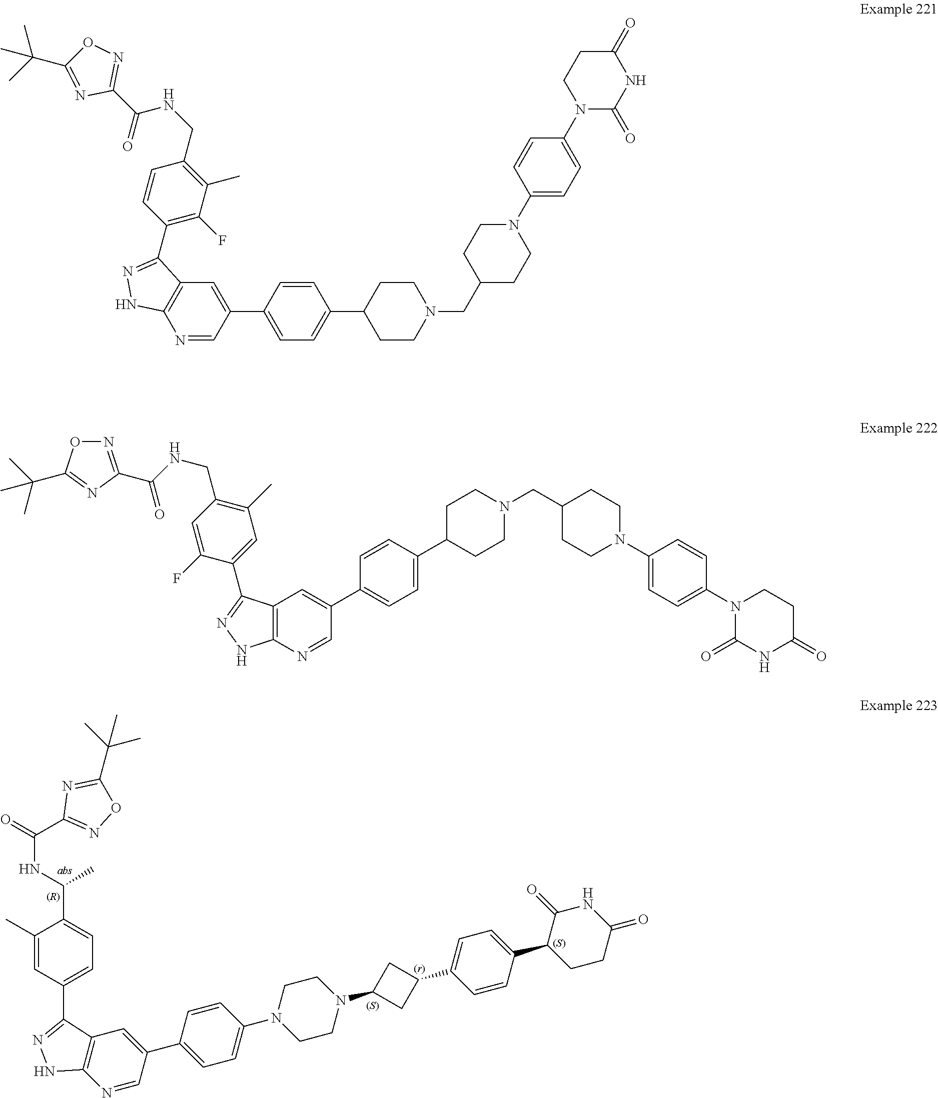

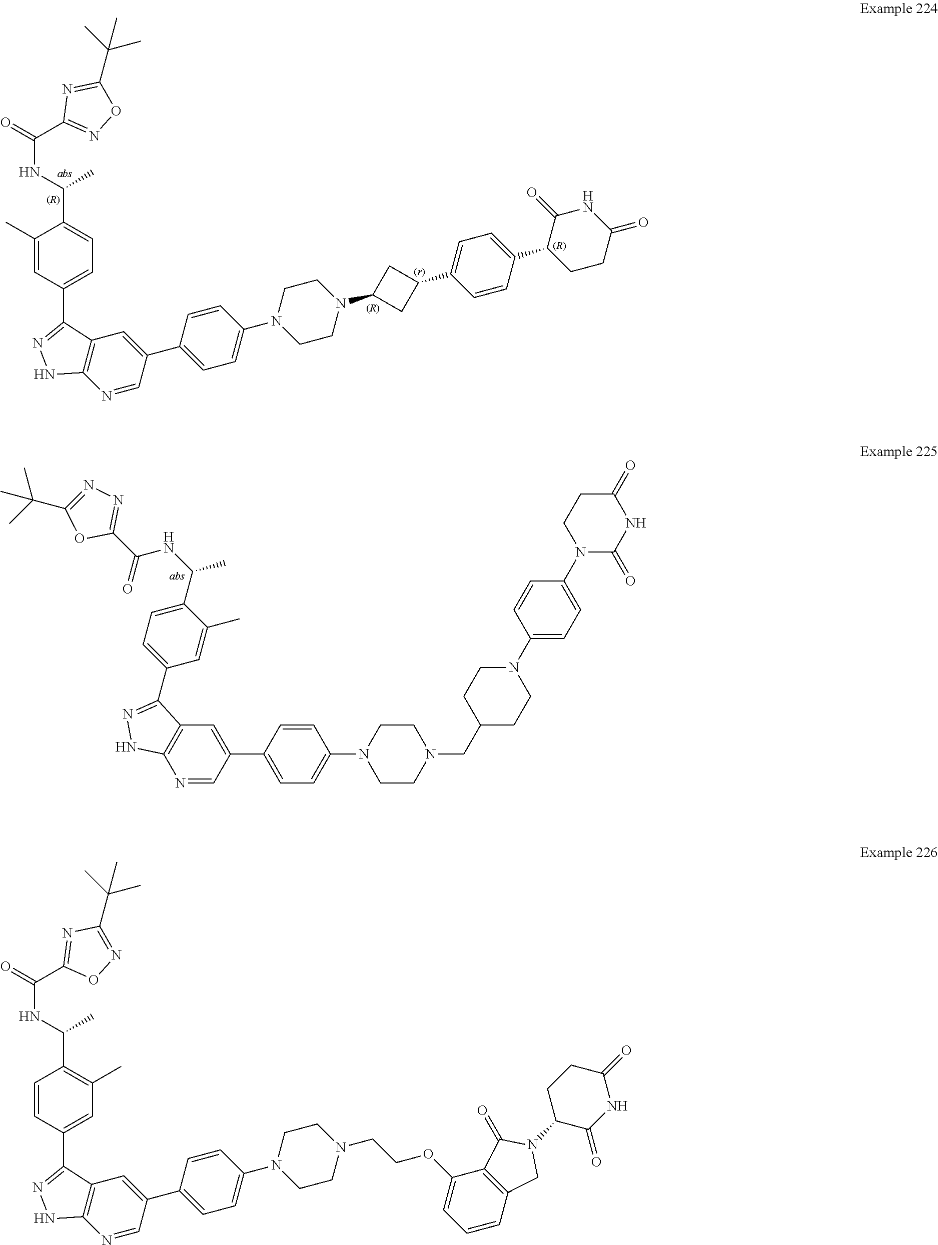

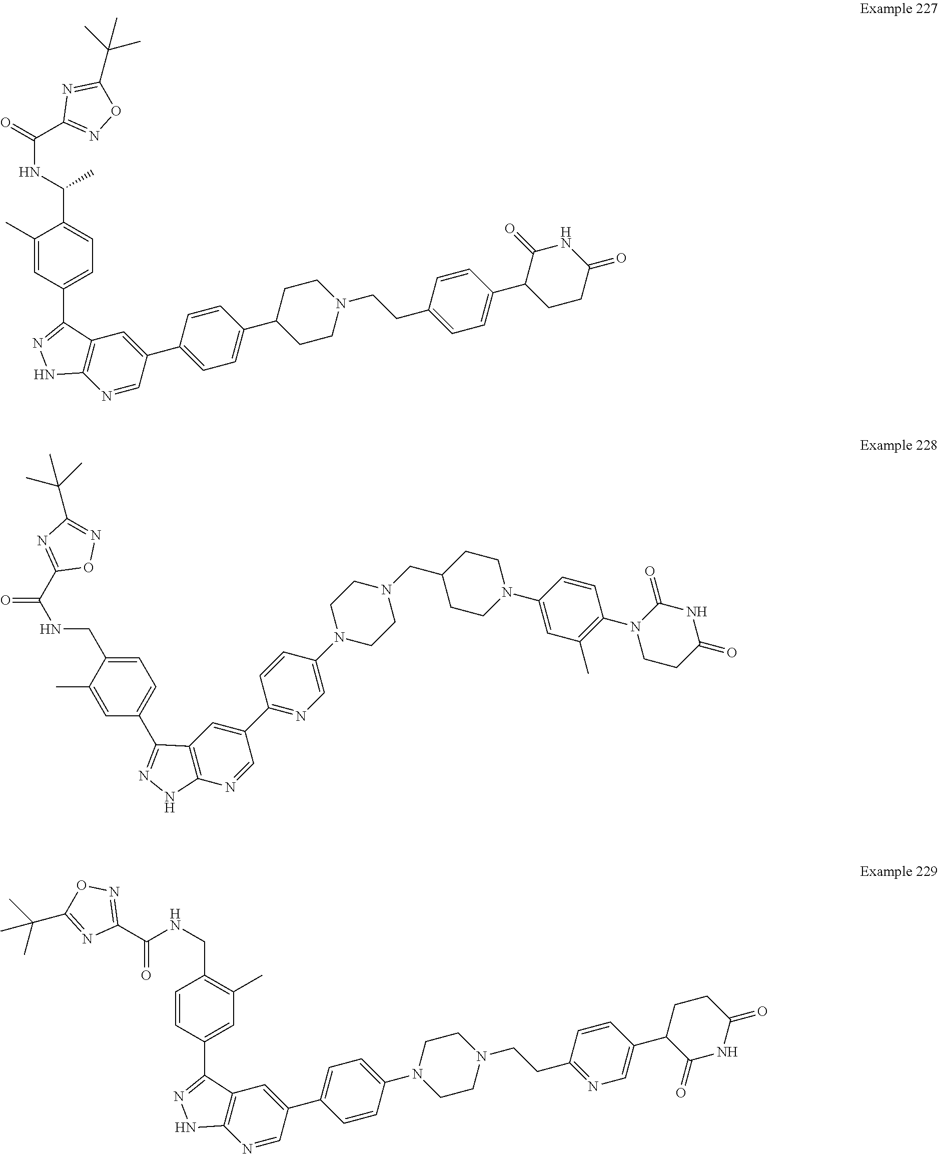 [SMILES: Cc1cc(-c2n[nH]c3ncc(-c4ccc(C5CCN(CCc6ccc(C7CCC(=O)NC7=O)cc6)CC5)cc4)cc23)ccc1[C@@H](C)NC(=O)c1noc(C(C)(C)C)n1 ; Cc1cc(-c2n[nH]c3ncc(-c4ccc(N5CCN(CC6CCN(c7ccc(N8CCC(=O)NC8=O)c(C)c7)CC6)CC5)cn4)cc23)ccc1CNC(=O)c1nc(C(C)(C)C)no1 ; Cc1cc(-c2n[nH]c3ncc(-c4ccc(N5CCN(CCc6ccc(C7CCC(=O)NC7=O)cn6)CC5)cc4)cc23)ccc1CNC(=O)c1noc(C(C)(C)C)n1]

In the second aspect, disclosed herein is a pharmaceutical composition comprising the compound disclosed herein, or a pharmaceutically acceptable salt thereof, and at least one pharmaceutically acceptable carrier or excipient.

In the third aspect, disclosed herein is a method of decreasing BTK activity by inhibition and/or protein degradation, which comprises administering to an individual the compound disclosed herein, or a pharmaceutically acceptable salt thereof, including the compound of formula (I) or the specific compounds exemplified herein.

In the fourth aspect, disclosed herein is a method of treating a disease or disorder in a patient comprising administering to the patient a therapeutically effective amount of the compound disclosed herein, or a pharmaceutically acceptable salt thereof as a BTK kinase inhibitor and/or degrader, wherein the compound disclosed herein includes the compound of formula (I) or the specific compounds exemplified herein. In some embodiments, the disease or disorder is associated with inhibition of BTK. Preferably, the disease or disorder is cancer.

### DETAILED DESCRIPTION OF THE INVENTION

### Definitions

The following terms have the indicated meaning throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” refers to a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include without limitation to methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1, 1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2, 3-dimethyl-2-butyl and 3, 3-dimethyl-2-butyl groups.

The term “propyl” refers to 1-propyl or n-propyl (“n-Pr”), 2-propyl or isopropyl (“i-Pr”).

The term “butyl” refers to 1-butyl or n-butyl (“n-Bu”), 2-methyl-1-propyl or isobutyl (“i-Bu”), 1-methylpropyl or s-butyl (“s-Bu”), 1, 1-dimethylethyl or t-butyl (“t-Bu”).

The term “pentyl” refers to 1-pentyl, 2-pentyl, 3-pentyl, 2-methyl-2-butyl, 3-methyl-2-butyl, 3-methyl-1-butyl, 2-methyl-1-butyl.

The term “hexyl” refers to 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2, 3-dimethyl-2-butyl and 3, 3-dimethyl-2-butyl.

The term “halogen” refers to fluoro (F), chloro (Cl), bromo (Br) and iodo (I).

The term “haloalkyl” refers to an alkyl group in which one or more hydrogens are replaced by one or more halogen atoms such as fluoro, chloro, bromo, and iodo. Examples of the haloalkyl include without limitation to haloC₁₋₈alkyl, haloC₁₋₆alkyl or halo C₁₋₄alkyl, such as —CF₃, —CH₂Cl, —CH₂CF₃, —CHCl₂, —CF₃, and the like.

The term “alkenyl” refers to a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include without limitation to ethenyl or vinyl, prop-1-enyl, prop-2-enyl, 2-methylprop-1-enyl, but-1-enyl, but-2-enyl, but-3-enyl, buta-1, 3-dienyl, 2-methylbuta-1, 3-dienyl, hex-1-enyl, hex-2-enyl, hex-3-enyl, hex-4-enyl, and hexa-1, 3-dienyl groups.

The term “alkynyl” refers to a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include without limitation to ethynyl, 1-propynyl, 2-propynyl (propargyl), 1-butynyl, 2-butynyl, and 3-butynyl groups.

The term “cycloalkyl” refers to a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include without limitation to cyclopropyl, cyclobutyl, cyclopentyl, cyclohexyl, cycloheptyl, and cyclooctyl groups. In a preferred embedment, the cycloalkyl is a monocyclic ring comprising 3 to 6 carbon atoms (abbreviated as C₃₋₆ cycloalkyl), including but not limited to cyclopropyl, cyclobutyl, cyclopentyl, and cyclohexyl. Examples of the bicyclic cycloalkyl groups include those having from 7 to 12 ring atoms arranged as a fused bicyclic ring selected from [4, 4], [4, 5], [5, 5], [5, 6] and [6, 6] ring systems, or as a bridged bicyclic ring selected from bicyclo[2.2.1]heptane, bicyclo[2.2.2]octane, and bicyclo[3.2.2]nonane. Further examples of the bicyclic cycloalkyl groups include those arranged as a bicyclic ring selected from [5, 6] and [6, 6] ring systems.

The term “spiro cycloalkyl” refers to a cyclic structure which contains carbon atoms and is formed by at least two rings sharing one atom. The term “7 to 12 membered spiro cycloalkyl” refers to a cyclic structure which contains 7 to 12 carbon atoms and is formed by at least two rings sharing one atom.

The term “fused cycloalkyl” refers to a bicyclic cycloalkyl group as defined herein which is saturated and is formed by two or more rings sharing two adjacent atoms.

The term “bridged cycloalkyl” refers to a cyclic structure which contains carbon atoms and is formed by two rings sharing two atoms which are not adjacent to each other. The term “7 to 10 membered bridged cycloalkyl” refers to a cyclic structure which contains 7 to 12 carbon atoms and is formed by two rings sharing two atoms which are not adjacent to each other.

The term “cycloalkenyl” refers to non-aromatic cyclic alkyl groups of from 3 to 10 carbon atoms having single or multiple rings and having at least one double bond and preferably from 1 to 2 double bonds. In one embodiment, the cycloalkenyl is cyclopentenyl or cyclohexenyl, 1-cyclopent-1-enyl, 1-cyclopent-2-enyl, 1-cyclopent-3-enyl, 1-cyclohex-1-enyl, 1-cyclohex-2-enyl, 1-cyclohex-3-enyl, cyclohexadienyl, preferably cyclohexenyl.

The term “fused cycloalkenyl” refers to a bicyclic cycloalkyl group as defined herein which contain at least one double bond and is formed by two or more rings sharing two adjacent atoms.

The term “cycloalkynyl” refers to non-aromatic cycloalkyl groups of from 5 to 10 carbon atoms having single or multiple rings and having at least one triple bond.

The term “fused cycloalkynyl” refers to a bicyclic cycloalkyl group as defined herein which contains at least one triple bond and is formed by two or more rings sharing two adjacent atoms.

The term a “benzo fused cycloalkyl” is a bicyclic fused cycloalkyl in which a 4- to 8-membered monocyclic cycloalkyl ring fused to a benzene ring. For example, a benzo fused cycloalkyl is

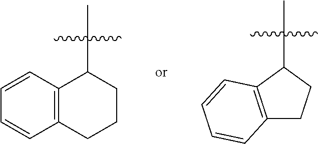 [SMILES: *C1CCCc2ccccc21 ; *C1CCc2ccccc21]

wherein the wavy lines indicate the points of attachment.

The term a “benzo fused cycloalkenyl” is a bicyclic fused cycloalkenyl in which a 4- to 8-membered monocyclic cycloalkenyl ring fused to a benzene ring.

The term a “benzo fused cycloalkynyl” is a bicyclic fused cycloalkynyl in which a 4- to 8-membered monocyclic cycloalkynyl ring fused to a benzene ring.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄₆ cycloalkenyl, 2, 3-dihydro-1H-indenyl, 1H-indenyl, 1, 2, 3, 4-tetralyl, 1, 4-dihydronaphthyl, etc. Preferred embodiments are 8 to 9 membered fused rings, which refer to cyclic structures containing 8 to 9 ring atoms within the above examples.

The term “aryl” used alone or in combination with other terms refers to a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeable throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include without limitation to phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” refers to a bicyclic aryl ring as defined herein. The typical bicyclic fused aryl is naphthalene.

The term “heteroaryl” refers to a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” refers to a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

Representative examples of bicyclic fused heteroaryl include without limitation to the following groups: benzisoxazolyl, benzodiazolyl, benzofuranyl, benzofurazanyl, benzofuryl, benzoimidazolyl, benzoisothiazolyl, benzothiadiazolyl, benzothiazolyl, benzothienyl, benzothiophenyl, benzotriazolyl, benzoxadiazolyl, benzoxazolyl, furopyridinyl, furopyrrolyl, imidazopyridinyl, imidazopyridyl, imidazothiazolyl, indazolyl, indolizinyl, indolyl, isobenzofuryl, isoindolyl, isoquinolinyl (or isoquinolyl), naphthyridinyl, phthalazinyl, pteridinyl, purinyl, pyrazinopyridazinyl, pyrazolopyridinyl, pyrazolopyrimidinyl, pyrazolopyridyl, pyrazolotriazinyl, pyridazolopyridyl, pyrrolopyridinyl, quinazolinyl, quinolinyl (or quinolyl), quinoxalinyl, thiazolopyridyl, thienopyrazinyl, thienopyrazolyl, thienopyridyl, thienopyrrolyl, thienothienyl, or triazolopyridyl.

The term a “benzo fused heteroaryl” is a bicyclic fused heteroaryl in which a 5- to 7-membered (preferably, 5- or 6-membered) monocyclic heteroaryl ring as defined herein fused to a benzene ring.

The terms “aromatic heterocyclic ring” and “heteroaryl” are used interchangeably throughout the disclosure herein. In some embodiments, a monocyclic or bicyclic aromatic heterocyclic ring has 5-, 6-, 7-, 8-, 9- or 10-ring forming members with 1, 2, 3, or 4 heteroatom ring members independently selected from nitrogen (N), sulfur (S) and oxygen (O) and the remaining ring members being carbon. In some embodiments, the monocyclic or bicyclic aromatic heterocyclic ring is a monocyclic or bicyclic ring comprising 1 or 2 heteroatom ring members independently selected from nitrogen (N), sulfur (S) and oxygen (O). In some embodiments, the monocyclic or bicyclic aromatic heterocyclic ring is a 5- to 6-membered heteroaryl ring, which is monocyclic and which has 1 or 2 heteroatom ring members independently selected from nitrogen (N), sulfur (S) and oxygen (O). In some embodiments, the monocyclic or bicyclic aromatic heterocyclic ring is an 8- to 10-membered heteroaryl ring, which is bicyclic and which has 1 or 2 heteroatom ring members independently selected from nitrogen, sulfur and oxygen.

Examples of the heteroaryl group or the monocyclic or bicyclic aromatic heterocyclic ring include, but are not limited to, (as numbered from the linkage position assigned priority 1) pyridyl (such as 2-pyridyl, 3-pyridyl, or 4-pyridyl), cinnolinyl, pyrazinyl, 2, 4-pyrimidinyl, 3, 5-pyrimidinyl, 2, 4-imidazolyl, imidazopyridinyl, isoxazolyl, oxazolyl, thiazolyl, isothiazolyl, thiadiazolyl (such as 1, 2, 3-thiadiazolyl, 1, 2, 4-thiadiazolyl, or 1, 3, 4-thiadiazolyl), tetrazolyl, thienyl (such as thien-2-yl, thien-3-yl), triazinyl, benzothienyl, furyl or furanyl, benzofuryl, benzoimidazolyl, indolyl, isoindolyl, oxadiazolyl (such as 1, 2, 3-oxadiazolyl, 1, 2, 4-oxadiazolyl, or 1, 3, 4-oxadiazolyl), phthalazinyl, pyrazinyl, pyridazinyl, pyrrolyl, triazolyl (such as 1, 2, 3-triazolyl, 1, 2, 4-triazolyl, or 1, 3, 4-triazolyl), quinolinyl, isoquinolinyl, pyrazolyl, pyrrolopyridinyl (such as 1H-pyrrolo[2, 3-b]pyridin-5-yl), pyrazolopyridinyl (such as 1H-pyrazolo[3, 4-b]pyridin-5-yl), benzoxazolyl (such as benzo[d]oxazol-6-yl), pteridinyl, purinyl, 1-oxa-2, 3-diazolyl, 1-oxa-2, 4-diazolyl, 1-oxa-2, 5-diazolyl, 1-oxa-3, 4-diazolyl, 1-thia-2, 3-diazolyl, 1-thia-2, 4-diazolyl, 1-thia-2, 5-diazolyl, 1-thia-3, 4-diazolyl, furazanyl (such as furazan-2-yl, furazan-3-yl), benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), and indazolyl (such as 1H-indazol-5-yl).

“Heterocyclyl”, “heterocycle” or “heterocyclic” are interchangeable and refer to a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “optionally oxidized sulfur” used herein refer to S, SO or SO₂.

The term “monocyclic heterocyclyl” refers to monocyclic groups in which at least one ring member (e.g., 1-3 heteroatoms, 1 or 2 heteroatom(s)) is a heteroatom selected from nitrogen, oxygen or optionally oxidized sulfur. A heterocycle may be saturated or partially saturated.

Exemplary monocyclic 4 to 9-membered heterocyclyl groups include without limitation to pyrrolidin-1-yl, pyrrolidin-2-yl, pyrrolidin-3-yl, imidazolidin-2-yl, imidazolidin-4-yl, pyrazolidin-2-yl, pyrazolidin-3-yl, piperidin-1-yl, piperidin-2-yl, piperidin-3-yl, piperidin-4-yl, 2, 5-piperazinyl, pyranyl, morpholinyl, morpholino, morpholin-2-yl, morpholin-3-yl, oxiranyl, aziridin-1-yl, aziridin-2-yl, azocan-1-yl, azocan-2-yl, azocan-3-yl, azocan-4-yl, azocan-5-yl, thiiranyl, azetidin-1-yl, azetidin-2-yl, azetidin-3-yl, oxetanyl, thietanyl, 1, 2-dithietanyl, 1, 3-dithietanyl, dihydropyridinyl, tetrahydropyridinyl, thiomorpholinyl, thioxanyl, piperazinyl, homopiperazinyl, homopiperidinyl, azepan-1-yl, azepan-2-yl, azepan-3-yl, azepan-4-yl, oxepanyl, thiepanyl, 1, 4-oxathianyl, 1, 4-dioxepanyl, 1, 4-oxathiepanyl, 1, 4-oxaazepanyl, 1, 4-dithiepanyl, 1, 4-thiazepanyl and 1, 4-diazepanyl, 1, 4-dithianyl, 1, 4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1, 4-dioxanyl, 1, 3-dioxolanyl, pyrazolinyl, pyrazolidinyl, dithianyl, dithiolanyl, pyrazolidinyl, imidazolinyl, pyrimidinonyl, or 1, 1-dioxo-thiomorpholinyl.

The term “spiro heterocyclyl” refers to a 5 to 20-membered polycyclic heterocyclyl with rings connected through one common carbon atom (called a spiro atom), comprising one or more heteroatoms selected from nitrogen, oxygen or optionally oxidized sulfur as ring members, with the remaining ring members being carbon. One or more rings of a spiro heterocyclyl group may contain one or more double bonds, but none of the rings has a completely conjugated pi-electron system. Preferably a spiro heterocyclyl is 6 to 14-membered, and more preferably 7 to 12-membered. According to the number of common spiro atoms, a spiro heterocyclyl could be mono-spiro heterocyclyl, di-spiro heterocyclyl, or poly-spiro heterocyclyl, and preferably refers to mono-spiro heterocyclyl or di-spiro heterocyclyl, and more preferably 4-membered/3-membered, 4-membered/4-membered, 3-membered/5-membered, 4-membered/5-membered, 4-membered/6-membered, 5-membered/5-membered, or 5-membered/6-membered mono-spiro heterocyclyl. Representative examples of spiro heterocyclyls include without limitation to the following groups: 2, 3-dihydrospiro[indene-1, 2′-pyrrolidine] (e.g., 2, 3-dihydrospiro[indene-1, 2′-pyrrolidine]-1′-yl), 1, 3-dihydrospiro[indene-2, 2′-pyrrolidine] (e.g., 1, 3-dihydrospiro[indene-2, 2′-pyrrolidine]-1′-yl), azaspiro[2.4]heptane (e.g., 5-azaspiro[2.4]heptane-5-yl), 2-oxa-6-azaspiro[3.3]heptane (e.g., 2-oxa-6-azaspiro[3.3]heptan-6-yl), azaspiro[3.4]octane (e.g., 6-azaspiro[3.4]octane-6-yl), 2-oxa-6-azaspiro[3.4]octane (e.g., 2-oxa-6-azaspiro[3.4]octane-6-yl), azaspiro[3.4]octane (e.g., 6-azaspiro[3.4]octan-6-yl), azaspiro[3.4]octane (e.g., 6-azaspiro[3.4]octan-6-yl), 1, 7-dioxaspiro[4.5]decane, 2-oxa-7-aza-spiro[4.4]nonane (e.g., 2-oxa-7-aza-spiro[4.4]non-7-yl), 7-oxa-spiro[3.5]nonyl and 5-oxa-spiro[2.4]heptyl.

The term “fused heterocyclyl” refers to a 5 to 20-membered polycyclic heterocyclyl group, wherein each ring in the system shares an adjacent pair of atoms (carbon and carbon atoms or carbon and nitrogen atoms) with another ring, comprising one or more heteroatoms selected from nitrogen, oxygen or optionally oxidized sulfur as ring members, with the remaining ring members being carbon. One or more rings of a fused heterocyclic group may contain one or more double bonds, but the fused heterocyclic group does not have a completely conjugated pi-electron system. Preferably, a fused heterocyclyl is 6 to 14-membered, and more preferably 7 to 12-membered, or 7- to 10-membered. According to the number of membered rings, a fused heterocyclyl could be bicyclic, tricyclic, tetracyclic, or polycyclic fused heterocyclyl. The group can be attached to the remainder of the molecule through either ring.

Specifically, the term “bicyclic fused heterocyclyl” refers to a 7 to 12-membered, preferably 7- to 10-membered, more preferably 9- or 10-membered fused heterocyclyl as defined herein comprising two fused rings and comprising 1 to 4 heteroatoms selected from nitrogen, oxygen or optionally oxidized sulfur as ring members. Typically, a bicyclic fused heterocyclyl is 5-membered/5-membered, 5-membered/6-membered, 6-membered/6-membered, or 6-membered/7-membered bicyclic fused heterocyclyl. Representative examples of (bicyclic) fused heterocycles include without limitation to the following groups: octahydrocyclopenta[c]pyrrole, octahydropyrrolo[3, 4-c]pyrrolyl, octahydroisoindolyl, isoindolinyl, octahydro-benzo[b][1, 4]dioxin, indolinyl, isoindolinyl, benzopyranyl, dihydrothiazolopyrimidinyl, tetrahydroquinolyl, tetrahydroisoquinolyl (or tetrahydroisoquinolinyl), dihydrobenzofuranyl, dihydrobenzoxazinyl, dihydrobenzoimidazolyl, tetrahydrobenzothienyl, tetrahydrobenzofuranyl, benzodioxolyl, benzodioxonyl, chromanyl, chromenyl, octahydrochromenyl, dihydrobenzodioxynyl, dihydrobenzoxezinyl, dihydrobenzodioxepinyl, dihydrothienodioxynyl, dihydrobenzooxazepinyl, tetrahydrobenzooxazepinyl, dihydrobenzoazepinyl, tetrahydrobenzoazepinyl, isochromanyl, chromanyl, or tetrahydropyrazolopyrimidinyl (e.g., 4, 5, 6, 7-tetrahydropyrazolo[1, 5-a]pyrimidin-3-yl).

The term a “benzo fused heterocyclyl” is a bicyclic fused heterocyclyl in which a monocyclic 4 to 9-membered heterocyclyl as defined herein (preferably 5- or 6-membered) fused to a benzene ring.

The term “bridged heterocyclyl” refers to a 5 to 14-membered polycyclic heterocyclic alkyl group, wherein every two rings in the system share two disconnected atoms, comprising one or more heteroatoms selected from nitrogen, oxygen or optionally oxidized sulfur as ring members, with the remaining ring members being carbon. One or more rings of a bridged heterocyclyl group may contain one or more double bonds, but none of the rings has a completely conjugated pi-electron system. Preferably, a bridged heterocyclyl is 6 to 14-membered, and more preferably 7 to 10-membered. According to the number of membered rings, a bridged heterocyclyl could be bicyclic, tricyclic, tetracyclic or polycyclic bridged heterocyclyl, and preferably refers to bicyclic, tricyclic or tetracyclic bridged heterocyclyl, and more preferably bicyclic or tricyclic bridged heterocyclyl. Representative examples of bridged heterocyclyls include without limitation to the following groups: 2-azabicyclo[2.2.1]heptyl, azabicyclo[3.1.0]hexyl, 2-azabicyclo[2.2.2]octyl and 2-azabicyclo[3.3.2]decyl.

The term “at least one substituent” disclosed herein includes, for example, from 1 to 4, such as from 1 to 3, further as 1 or 2, substituents, provided that the theory of valence is met. For example, “at least one substituent R^{6d}” disclosed herein includes from 1 to 4, such as from 1 to 3, further as 1 or 2, substituents selected from the list of R^{6d} as disclosed herein.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical or chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers and diastereomers can also be separated by the use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

“Pharmaceutically acceptable salts” refer to those salts which are, within the scope of sound medical judgment, suitable for use in contact with the tissues of humans and animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “a pharmaceutically acceptable salt thereof” includes salts of at least one compound of Formula (I), and salts of the stereoisomers of the compound of Formula (I), such as salts of enantiomers, and/or salts of diastereomers.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic agent, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer thereof, and/or at least one pharmaceutically acceptable salt thereof disclosed herein effective to “treat” as defined herein, a disease or disorder in a subject. In the case of combination therapy, the “therapeutically effective amount” refers to the total amount of the combination objects for the effective treatment of a disease, a disorder or a condition.

The pharmaceutical composition comprising the compound disclosed herein can be administrated via oral, inhalation, rectal, parenteral or topical route to a subject in need thereof. For oral administration, the pharmaceutical composition may be a regular solid formulation such as tablets, powder, granule, capsules and the like, a liquid formulation such as water or oil suspension or other liquid formulation such as syrup, solution, suspension or the like; for parenteral administration, the pharmaceutical composition may be solution, water solution, oil suspension concentrate, lyophilized powder or the like. Preferably, the formulation of the pharmaceutical composition is selected from tablet, coated tablet, capsule, suppository, nasal spray or injection, more preferably tablet or capsule. The pharmaceutical composition can be a single unit administration with an accurate dosage. In addition, the pharmaceutical composition may further comprise additional active ingredients.

All formulations of the pharmaceutical composition disclosed herein can be produced by the conventional methods in the pharmaceutical field. For example, the active ingredient can be mixed with one or more excipients, then to make the desired formulation. The “pharmaceutically acceptable excipient” refers to conventional pharmaceutical carriers suitable for the desired pharmaceutical formulation, for example: a diluent, a vehicle such as water, various organic solvents, etc., a filler such as starch, sucrose, etc., a binder such as cellulose derivatives, alginates, gelatin and polyvinylpyrrolidone (PVP); a wetting agent such as glycerol; a disintegrating agent such as agar, calcium carbonate and sodium bicarbonate; an absorption enhancer such as quaternary ammonium compound; a surfactant such as hexadecanol; an absorption carrier such as Kaolin and soap clay; a lubricant such as talc, calcium stearate, magnesium stearate, polyethylene glycol, etc. In addition, the pharmaceutical composition further comprises other pharmaceutically acceptable excipients such as a decentralized agent, a stabilizer, a thickener, a complexing agent, a buffering agent, a permeation enhancer, a polymer, an aromatic, a sweetener, a dye and etc.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

General Reaction Scheme for Compound Preparation

The subject compounds and pharmaceutically acceptable salts thereof, can be prepared from (a) commercially available starting materials (b) known starting materials which may be prepared as described in literature procedures (c) new intermediates described in the schemes and experimental procedures herein. In making the compounds of the invention, the order of synthetic steps may be varied to increase the yield of desired product. Some of compounds in this invention may be generated by the methods as shown in the following reaction schemes and the description thereof.

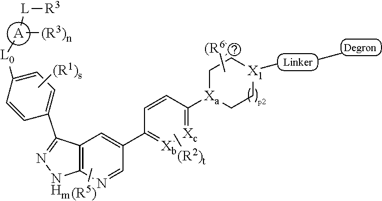

Wherein Xm, Xn are I, Br, Cl and H; P is protective group, such as Boc, THP, SEM; R⁵, R³, R⁴, R₁, Xa, Xb, and Xc are defined as described herein. I-3 could be synthesized from I-1 and I-2 in the presence of metal catalyst in the basic condition, then I-3 was coupled with I-4 in basic condition and also with metal as catalyst to form I-5. Then the protective group was removed in acid condition to give I-6, which was converted to compound I with SN2 reaction, or reductive amination, or coupling reaction.

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on a Agilent instrument operating at 400 MHz. ¹HNMR

spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LC-MS spectrometer (Agilent 1260) Detector: MWD (190-400 nm), Mass detector: 6120 SQ Mobile phase: A: acetonitrile with 0.1% Formic acid, B: water with 0.1% Formic acid Column: Poroshell 120 EC-C18, 4.6×50 mm, 2.7 pm Gradient method; Flow: 1.8 mL/min Time (min) A (%) B (%)

| Time | A | B |
|---|---|---|
| (min) | (%) | (%) |
| 0.00 | 5 | 95 |
| 1.5 | 95 | 5 |
| 2.0 | 95 | 5 |
| 2.1 | 5 | 95 |
| 3.0 | 5 | 95 |

Preparative HPLC was conducted on a column (150×21.2 mm ID, 5 pm, Gemini NXC 18) at a flow rate of 20 ml/min, injection volume 2 ml, at room temperature and UV Detection at 214 nm and 254 nm.

In the following examples, the abbreviations below are used:

| | |
|---|---|
| AcOH | Acetic acid |
| Aq | Aqueous |
| Brine | Saturated aqueous sodium chloride solution |
| Bn | Benzyl |
| Boc | Tert-butyloxycarbonyl |
| CH₂Cl₂ | Dichloromethane |
| DMF | N, N-Dimethylformamide |
| Dppf | 1, 1″-bis(diphenylphosphino)ferrocene |
| DCM | Dichloromethane |
| DIEA or DIPEA | N, N-diisopropylethylamine |
| DMF | N, N-dimethylformamide |
| DMSO | Dimethyl sulfoxide |
| EA or EtOAc | Ethyl acetate |
| FA | Formic acid |
| FBS | Fetal Bovine Serum |
| g | Grams |
| h or hr | Hour |
| HATU | O-(7-Azabenzotriazol-1-yl)-N, N, N′, N′-tetramethyluronium hexafluorophosphate |
| HCl | Hydrochloric acid |
| Hex | Hexane |
| HPLC | High-performance liquid chromatography |
| IBX | 2-Iodoxybenzoic acid |
| IPA | 2-propanol |
| i-PrOH | Isopropyl alcohol |
| mg | Milligrams |
| mL | Milliliters |
| mmol | Millimole |
| MeOH | Methanol |
| Min | Minutes |
| ms or MS | Mass spectrum |
| Na₂SO₄ | Sodium sulfate |
| PE | Petroleum ether |
| PS | Penicillin and strepto-mycin |
| PyBOP | (Benzotriazol-1-yloxy)tripyrrolidinophosphonium hexafluorophosphate |
| Rt | Retention time |
| R.T. or r.t. | Room temperature |
| TEA | triethylamine |
| TFA | Trifluoroacetic acid |
| THE | Tetrahydrofuran |
| THP | Tetrahydropyran |
| TLC | thin layer chromatography |
| μL | Microliters |
| X-Phos | Dicyclohexyl(2′,4′,6′-triisopropyl-2-biphenylyl)phosphine |
| RuPhos-Pd-G3 | Methanesulfonato(2-dicyclohexylphosphino-2′,6′-di-i-propoxy-1,1′-biphenyl) |
| | (2′-amino-1,1′-biphenyl-2-yl)palladium(II) |
| MTBE | Methyl tert-butyl ether |
| NMP | N-Methyl-2-pyrrolidone |
| STAB | Sodium triacetoxyborohydride |
| DAST | Diethylaminosulfur trifluoride |

### Example 1: 5-(tert-butyl)-N-(4-(5-(4-(1-((1-(4-(2,4-dioxotetrahydropyrimidin-1(2H)-yl)phenyl)piperidin-4-yl)methyl)piperidin-4-yl)phenyl)-1H-pyrazolo[3,4-b]pyridin-3-yl)-2-methylbenzyl)-1,2,4-oxadiazole-3-carboxamide

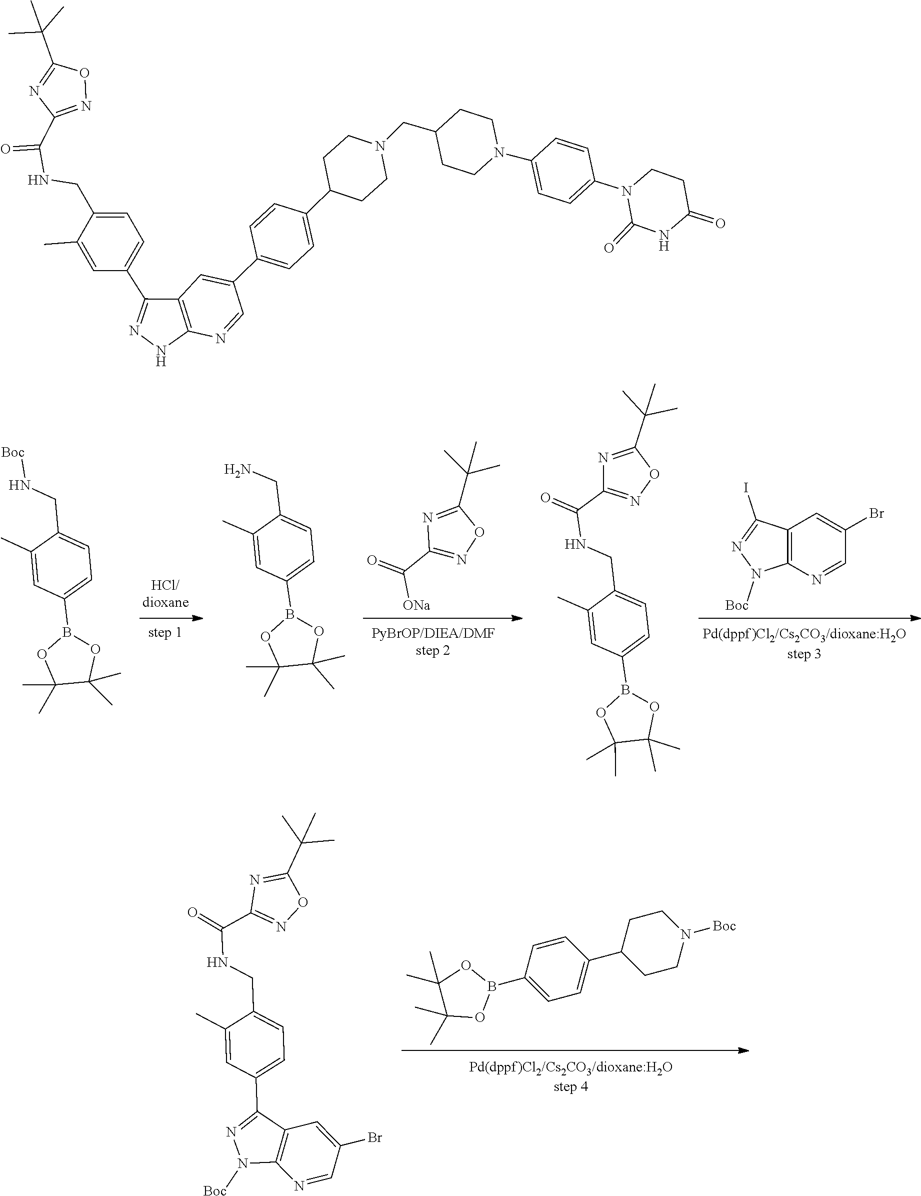

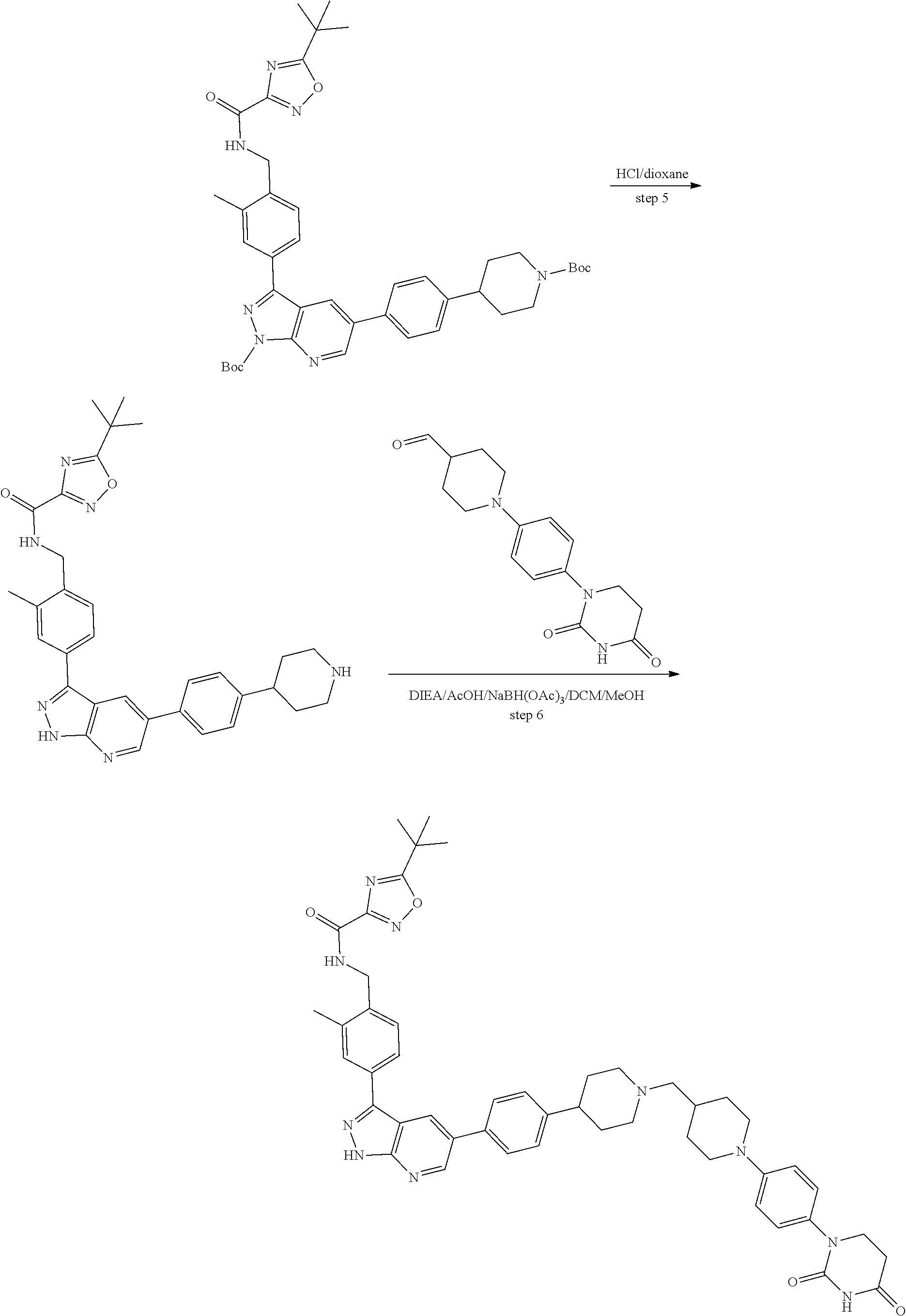 [SMILES: Cc1cc(-c2nn(C(=O)OC(C)(C)C)c3ncc(-c4ccc(C5CCN(C(=O)OC(C)(C)C)CC5)cc4)cc23)ccc1CNC(=O)c1noc(C(C)(C)C)n1 ; Cc1cc(-c2n[nH]c3ncc(-c4ccc(C5CCNCC5)cc4)cc23)ccc1CNC(=O)c1noc(C(C)(C)C)n1 ; O=CC1CCN(c2ccc(N3CCC(=O)NC3=O)cc2)CC1 ; Cc1cc(-c2n[nH]c3ncc(-c4ccc(C5CCN(CC6CCN(c7ccc(N8CCC(=O)NC8=O)cc7)CC6)CC5)cc4)cc23)ccc1CNC(=O)c1noc(C(C)(C)C)n1]

### Step 1: (2-methyl-4-(4,4,5,5-tetramethyl-1,3,2-dioxaborolan-2-yl)phenyl)methanamine

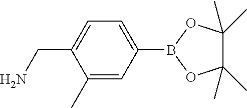 [SMILES: Cc1cc(B2OC(C)(C)C(C)(C)O2)ccc1CN]

A solution of tert-butyl (2-methyl-4-(4,4,5,5-tetramethyl-1,3,2-dioxaborolan-2-yl)benzyl)carbamate (1.05 g, 3.0 mmol) in HCl/dioxane (10 mL) was stirred at room temperature for 2 hours. After the reaction was completed, the solvent was removed under reduced pressure to give the desired product (850 mg, 99%). [M+H]⁺=247.2.

### Step 2: 5-(tert-butyl)-N-(2-methyl-4-(4,4,5,5-tetramethyl-1,3,2-dioxaborolan-2-yl)benzyl)-1,2,4-oxadiazole-3-carboxamide

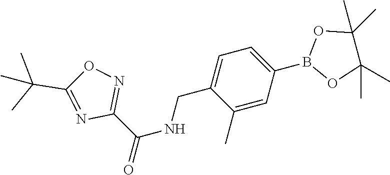 [SMILES: Cc1cc(B2OC(C)(C)C(C)(C)O2)ccc1CNC(=O)c1noc(C(C)(C)C)n1]

To a solution of (2-methyl-4-(4,4,5,5-tetramethyl-1,3,2-dioxaborolan-2-yl)phenyl)methanamine (850 mg, 3.0 mmol), sodium 5-(tert-butyl)-1,2,4-oxadiazole-3-carboxylate (860 mg, 4.5 mmol) and DIEA (1.2 g, 9.0 mmol) in DMF (10 mL) was added PyBOP (2.1 g, 4.5 mmol). The resulting mixture was stirred at room temperature for 1 h. The reaction was quenched with water and extracted with EtOAc. The combined organic layers were washed with brine, dried over anhydrous Na₂SO₄, and evaporated in vacuum to afford the crude product, which was further purified with silica gel column chromatography (PE:EtOAc=10:1˜2:1 gradient elution) to give the product (1.0 g, 73%). [M+H]+=400.2.

### Step 3: tert-butyl 5-bromo-3-(4-((5-(tert-butyl)-1,2,4-oxadiazole-3-carboxamido)methyl)-3-methylphenyl)-1H-pyrazolo[3,4-b]pyridine-1-carboxylate

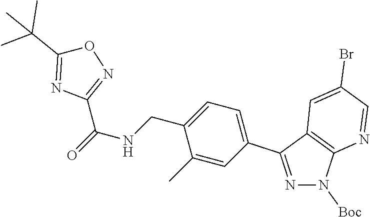 [SMILES: Cc1cc(-c2nn(C(=O)OC(C)(C)C)c3ncc(Br)cc23)ccc1CNC(=O)c1noc(C(C)(C)C)n1]

A mixture of tert-butyl 5-bromo-3-iodo-1H-pyrazolo[3,4-b]pyridine-1-carboxylate (550 mg, 1.3 mmol), 5-(tert-butyl)-N-(2-methyl-4-(4,4,5,5-tetramethyl-1,3,2-dioxaborolan-2-yl)benzyl)-1,2,4-oxadiazole-3-carboxamide (522 mg, 1.3 mmol), Pd(dppf)Cl₂ (95 mg, 0.13 mmol) and Cs₂CO₃ (638 mg, 1.95 mmol) in dioxane (15 mL)/H₂O (3 mL) was stirred in a round bottom flask at 80° C. for 1 h under N₂. The solvent was removed under reduced pressure and the crude product was purified with silica gel column chromatography (PE:EtOAc=10:1˜4:1 gradient elution) to give the product (390 mg, 53%). [M+H]⁺=569.1.

### Step 4: tert-butyl 5-(4-(1-(tert-butoxycarbonyl)piperidin-4-yl)phenyl)-3-(4-((5-(tert-butyl)-1,2,4-oxadiazole-3-carboxamido)methyl)-3-methylphenyl)-1H-pyrazolo[3,4-b]pyridine-1-carboxylate

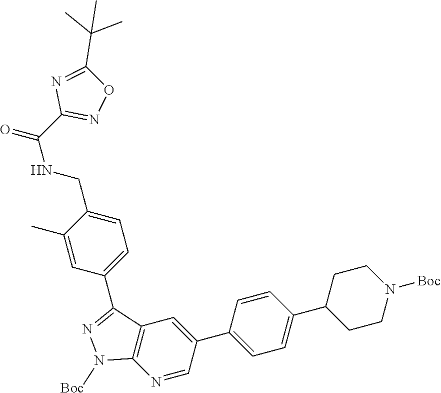 [SMILES: Cc1cc(-c2nn(C(=O)OC(C)(C)C)c3ncc(-c4ccc(C5CCN(C(=O)OC(C)(C)C)CC5)cc4)cc23)ccc1CNC(=O)c1noc(C(C)(C)C)n1]

A mixture of tert-butyl 5-bromo-3-(4-((5-(tert-butyl)-1,2,4-oxadiazole-3-carboxamido)methyl)-3-methylphenyl)-1H-pyrazolo[3,4-b]pyridine-1-carboxylate (390 mg, 0.68 mmol), tert-butyl 4-(4-(4,4,5,5-tetramethyl-1,3,2-dioxaborolan-2-yl)phenyl)piperidine-1-carboxylate (265 mg, 0.68 mmol), Pd(dppf)Cl₂ (50 mg, 0.068 mmol) and Cs₂CO₃ (335 mg, 1.03 mmol) in dioxane (15 mL)/H₂O (3 mL) was stirred in a round bottom flask at 90° C. for 3 h under N₂. The solvent was removed under reduced pressure and the crude product was purified with silica gel column chromatography (DCM:MeOH=100:1˜10:1 gradient elution) to give the product (270 mg, 53%). [M+H]⁺=750.5.

### Step 5: 5-(tert-butyl)-N-(2-methyl-4-(5-(4-(piperidin-4-yl)phenyl)-1H-pyrazolo[3,4-b]pyridin-3-yl)benzyl)-1,2,4-oxadiazole-3-carboxamide

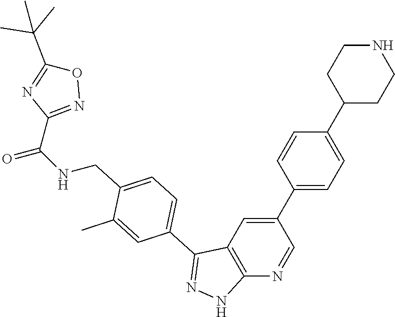 [SMILES: Cc1cc(-c2n[nH]c3ncc(-c4ccc(C5CCNCC5)cc4)cc23)ccc1CNC(=O)c1noc(C(C)(C)C)n1]

A solution of tert-butyl 5-(4-(1-(tert-butoxycarbonyl)piperidin-4-yl)phenyl)-3-(4-((5-(tert-butyl)-1,2,4-oxadiazole-3-carboxamido)methyl)-3-methylphenyl)-1H-pyrazolo[3,4-b]pyridine-1-carboxylate (270 mg, 0.36 mmol) in HCl/dioxane (10 mL) was stirred at room temperature for 2 hours. After the reaction was completed, the solvent was removed under reduced pressure to give the desired product (220 mg, 99%). [M+H]⁺=550.3.

### Step 6: 5-(tert-butyl)-N-(4-(5-(4-(1-((1-(4-(2,4-dioxotetrahydropyrimidin-1(2H)-yl)phenyl)piperidin-4-yl)methyl)piperidin-4-yl)phenyl)-1H-pyrazolo[3,4-b]pyridin-3-yl)-2-methylbenzyl)-1,2,4-oxadiazole-3-carboxamide

To a solution of 5-(tert-butyl)-N-(2-methyl-4-(5-(4-(piperidin-4-yl)phenyl)-1H-pyrazolo[3,4-b]pyridin-3-yl)benzyl)-1,2,4-oxadiazole-3-carboxamide (25 mg, 0.043 mmol), 1-(4-(2,4-dioxotetrahydropyrimidin-1(2H)-yl)phenyl)piperidine-4-carbaldehyde (20 mg, 0.064 mmol) and DIEA (6 mg, 0.043 mmol) in DCM (5 mL)/MeOH (1 mL) was added acetic acid (3 mg, 0.043 mmol). After stirring at room temperature for 0.5 h, NaBH(OAc)₃ (27 mg, 0.129 mmol) was added and the resulting mixture was stirred at room temperature for 2 h. The reaction was quenched with water and extracted with DCM. The organic layer was dried over anhydrous Na₂SO₄, and evaporated in vacuum to afford the crude product, which was further purified with pre-HPLC to give the product (8.2 mg, 21%). ¹H NMR (400 MHz, DMSO) δ_{H} 13.83 (s, 1H), 10.25 (s, 1H), 9.45 (s, 1H), 8.85 (s, 1H), 8.66 (s, 1H), 8.17 (s, 1H), 7.91 (d, J=12.5 Hz, 2H), 7.74 (d, J=7.7 Hz, 2H), 7.39 (d, J=8.0 Hz, 3H), 7.13 (d, J=8.9 Hz, 2H), 6.93 (d, J=8.6 Hz, 2H), 4.52 (d, J=5.7 Hz, 2H), 3.74-3.65 (m, 4H), 3.00 (d, J=10.7 Hz, 2H), 2.71-2.63 (m, 4H), 2.57 (s, 1H), 2.45 (s, 3H), 2.23 (d, J=6.8 Hz, 2H), 2.03 (s, 2H), 1.86-1.64 (m, 7H), 1.44 (s, 9H), 1.23 (d, J=12.5 Hz, 2H); [M+H]⁺=835.8.

### Example 2: 5-(tert-butyl)-N-(4-(5-(4-(1-(3-(4-((2,6-dioxopiperidin-3-yl)amino)phenoxy)propyl)piperidin-4-yl)phenyl)-1H-pyrazolo[3,4-b]pyridin-3-yl)-2-methylbenzyl)-1,2,4-oxadiazole-3-carboxamide

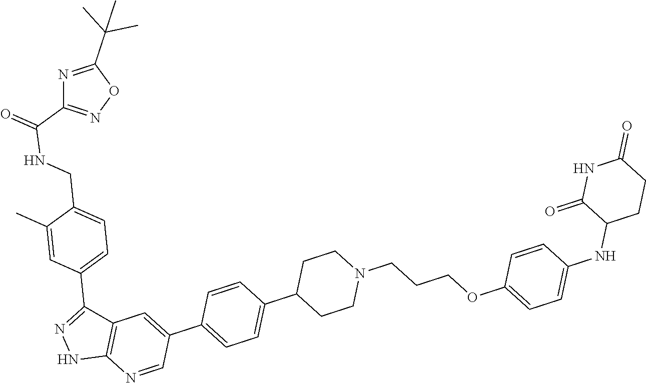 [SMILES: Cc1cc(-c2n[nH]c3ncc(-c4ccc(C5CCN(CCCOc6ccc(NC7CCC(=O)NC7=O)cc6)CC5)cc4)cc23)ccc1CNC(=O)c1noc(C(C)(C)C)n1]

Step 1: To a solution of 5-(tert-butyl)-N-(2-methyl-4-(5-(4-(piperidin-4-yl)phenyl)-1H-pyrazolo[3,4-b]pyridin-3-yl)benzyl)-1,2,4-oxadiazole-3-carboxamide (25 mg, 0.04 mmol) and 3-(4-((2,6-dioxopiperidin-3-yl)amino)phenoxy)propyl methanesulfonate (23 mg, 0.06 mmol) in DMSO (2 mL) was added DIEA (17 mg, 0.12 mmol). The resulting mixture was heated at 90° C. overnight. The reaction was directly purified with pre-HPLC to give the titled product (1.46 mg, 4.2%). ¹H NMR (400 MHz, DMSO) δ_{H} 13.83 (s, 1H), 10.76 (s, 1H), 9.45 (s, 1H), 8.84 (s, 1H), 8.65 (s, 1H), 7.89 (s, 2H), 7.74 (d, J=7.7 Hz, 2H), 7.39 (d, J=7.7 Hz, 3H), 6.73 (d, J=8.8 Hz, 2H), 6.63 (d, J=8.4 Hz, 2H), 5.43 (d, J=7.3 Hz, 1H), 4.52 (d, J=5.7 Hz, 2H), 4.20 (s, 1H), 3.91 (s, 2H), 3.01 (d, J=10.8 Hz, 2H), 2.73 (s, 1H), 2.58 (d, J=13.6 Hz, 1H), 2.45 (s, 5H), 2.15-2.01 (m, 3H), 1.90-1.68 (m, 7H), 1.44 (s, 9H); [M+H]+=810.6.

### Example 3: 5-(tert-butyl)-N-(4-(5-(4-(1-(4-(2,6-dioxopiperidin-3-yl)phenethyl)piperidin-4-yl)phenyl)-1H-pyrazolo[3,4-b]pyridin-3-yl)-2-methylbenzyl)-1,2,4-oxadiazole-3-carboxamide

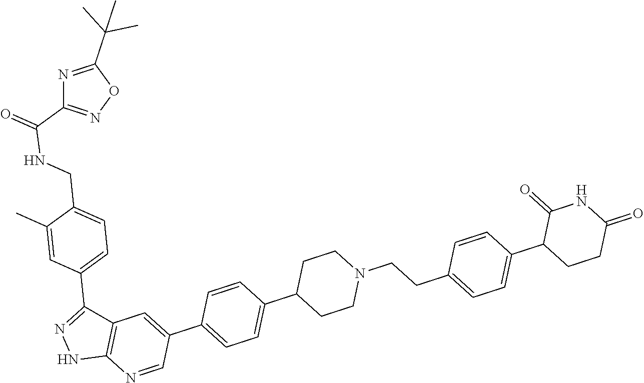 [SMILES: Cc1cc(-c2n[nH]c3ncc(-c4ccc(C5CCN(CCc6ccc(C7CCC(=O)NC7=O)cc6)CC5)cc4)cc23)ccc1CNC(=O)c1noc(C(C)(C)C)n1]

The titled compound was synthesized in the procedures similar to Example 1. ¹H NMR (400 MHz, DMSO) δ_{H} 13.84 (s, 1H), 10.82 (s, 1H), 9.45 (s, 1H), 8.84 (s, 1H), 8.66 (s, 1H), 7.89 (s, 2H), 7.74 (d, J=7.6 Hz, 2H), 7.40 (d, J=7.6 Hz, 3H), 7.18 (dd, J=30.2, 7.5 Hz, 4H), 4.52 (d, J=5.2 Hz, 2H), 3.82 (d, J=6.7 Hz, 1H), 3.09 (d, J=9.9 Hz, 2H), 2.76 (d, J=7.2 Hz, 2H), 2.70-2.53 (m, 5H), 2.45 (s, 3H), 2.22-2.00 (m, 4H), 1.85-1.70 (m, 4H), 1.44 (s, 9H); [M+H]⁺=765.6.

### Example 4: 3-(tert-butyl)-N-(4-(5-(4-(1-((1-(4-(2,4-dioxotetrahydropyrimidin-1(2H)-yl)phenyl)piperidin-4-yl)methyl)piperidin-4-yl)phenyl)-1H-pyrazolo[3,4-b]pyridin-3-yl)-2-methylbenzyl)-1,2,4-oxadiazole-5-carboxamide

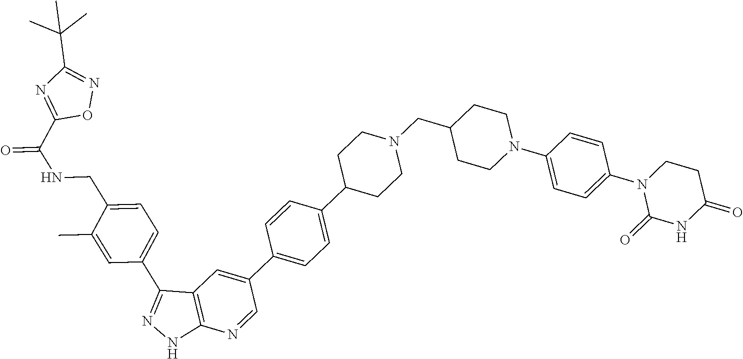 [SMILES: Cc1cc(-c2n[nH]c3ncc(-c4ccc(C5CCN(CC6CCN(c7ccc(N8CCC(=O)NC8=O)cc7)CC6)CC5)cc4)cc23)ccc1CNC(=O)c1nc(C(C)(C)C)no1]

### Step 1: N-(4-(5-bromo-1-(tetrahydro-2H-pyran-2-yl)-1H-pyrazolo[3,4-b]pyridin-3-yl)-2-methylbenzyl)-3-(tert-butyl)-1,2,4-oxadiazole-5-carboxamide

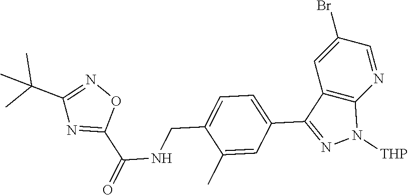

A mixture of 5-bromo-3-iodo-1-(tetrahydro-2H-pyran-2-yl)-1H-pyrazolo[3,4-b]pyridine (2.03 g, 5.0 mmol), 3-(tert-butyl)-N-(2-methyl-4-(4,4,5,5-tetramethyl-1,3,2-dioxaborolan-2-yl)benzyl)-1,2,4-oxadiazole-5-carboxamide (2.0 g, 5.0 mmol), Pd(dppf)Cl₂ (0.365 g, 0.5 mmol) and Cs₂CO₃ (3.25 g, 10 mmol) in dioxane (20 mL) was stirred in a round bottom flask at 80° C. overnight. Then the mixture was evaporated in vacuum to afford the crude product, which was further purified with silica gel column chromatography (PE:EtOAc=100:0˜30:70 gradient elution) to give the product (2.2 g, 80%). [M+H]⁺=553.4.

### Step 2: tert-butyl 4-(4-(3-(4-((3-(tert-butyl)-1,2,4-oxadiazole-5-carboxamido)methyl)-3-methylphenyl)-1-(tetrahydro-2H-pyran-2-yl)-1H-pyrazolo[3,4-b]pyridin-5-yl)phenyl)piperidine-1-carboxylate

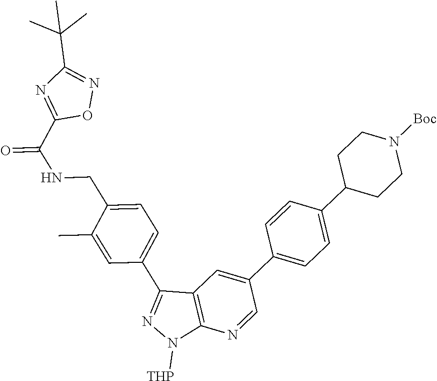

A mixture of N-(4-(5-bromo-1-(tetrahydro-2H-pyran-2-yl)-1H-pyrazolo[3,4-b]pyridin-3-yl)-2-methylbenzyl)-3-(tert-butyl)-1,2,4-oxadiazole-5-carboxamide (2.2 g, 3.98 mmol), tert-butyl 4-(4-(4,4,5,5-tetramethyl-1,3,2-dioxaborolan-2-yl)phenyl)piperidine-1-carboxylate (1.54 g, 3.98 mmol), Pd(dppf)Cl₂ (0.29 g, 0.398 mmol) and Cs₂CO₃ (2.6 g, 7.96 mmol) in dioxane (20 mL) was stirred in a round bottom flask at 100° C. overnight. Then the mixture was evaporated in vacuum to afford the crude product, which was further purified with silica gel column chromatography (PE:EtOAc=100:0˜20:80 gradient elution) to give the product (1.5 g, 51%). [M+H]⁺=734.5.

### Step 3: 3-(tert-butyl)-N-(2-methyl-4-(5-(4-(piperidin-4-yl)phenyl)-1H-pyrazolo[3,4-b]pyridin-3-yl)benzyl)-1,2,4-oxadiazole-5-carboxamide

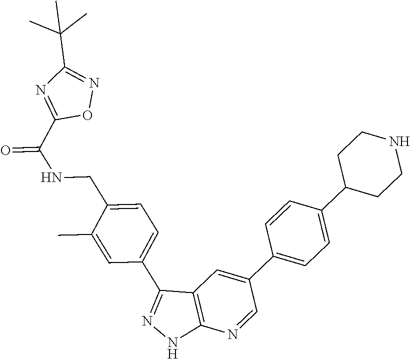 [SMILES: Cc1cc(-c2n[nH]c3ncc(-c4ccc(C5CCNCC5)cc4)cc23)ccc1CNC(=O)c1nc(C(C)(C)C)no1]

A mixture of tert-butyl 4-(4-(3-(4-((3-(tert-butyl)-1,2,4-oxadiazole-5-carboxamido)methyl)-3-methylphenyl)-1-(tetrahydro-2H-pyran-2-yl)-1H-pyrazolo[3,4-b]pyridin-5-yl)phenyl)piperidine-1-carboxylate (0.6 g, 0.8 mmol) and trifluoroacetic acid (3 mL) in dichloromethane (3 mL) was stirred in a round bottom flask at room temperature overnight. After the mixture was evaporated in vacuum to afford the crude product (0.5 g, 80%), which was used for next step without further purification. [M+H]⁺=550.3.

### Step 4: 3-(tert-butyl)-N-(4-(5-(4-(1-((1-(4-(2,4-dioxotetrahydropyrimidin-1(2H)-yl)phenyl)piperidin-4-yl)methyl)piperidin-4-yl)phenyl)-1H-pyrazolo[3,4-b]pyridin-3-yl)-2-methylbenzyl)-1,2,4-oxadiazole-5-carboxamide

A mixture of 3-(tert-butyl)-N-(2-methyl-4-(5-(4-(piperidin-4-yl)phenyl)-1H-pyrazolo[3,4-b]pyridin-3-yl)benzyl)-1,2,4-oxadiazole-5-carboxamide (183 mg, 0.33 mmol) and 1-(4-(2,4-dioxotetrahydropyrimidin-1(2H)-yl)phenyl)piperidine-4-carbaldehyde (100 mg, 0.33 mmol) in dichloromethane (20 mL) was stirred in a round bottom flask at room temperature for 1 hour. Then the mixture was added NaBH₃CN (44 mg, 0.66 mmol) and stirred in a round bottom flask at room temperature overnight. After the mixture was evaporated in vacuum to afford the crude product, which was purified with silica gel column chromatography (DCM:MeOH=100:0˜80:20 gradient elution) to give the product (50.5 mg, 18%). ¹H NMR (400 MHz, DMSO) δ_{H} 13.81 (s, 1H), 10.22 (s, 1H), 9.82 (s, 1H), 8.80 (s, 1H), 8.61 (s, 1H), 7.94-7.79 (m, 2H), 7.76-7.58 (m, 2H), 7.45-7.23 (m, 3H), 7.09 (d, J=7.2 Hz, 2H), 6.89 (d, J=6.8 Hz, 2H), 4.48 (s, 2H), 3.76-3.56 (m, 4H), 2.71-2.55 (m, 5H), 2.40 (s, 3H), 1.85-1.66 (m, 7H), 1.32 (s, 9H), 1.26-1.11 (m, 3H); [M+H]⁺=835.5.

### Example 5: 3-(tert-butyl)-N-(4-(5-(4-(1-(2-(1-(4-(2,4-dioxotetrahydropyrimidin-1(2H)-yl)phenyl)piperidin-4-yl)ethyl)piperidin-4-yl)phenyl)-1H-pyrazolo[3,4-b]pyridin-3-yl)-2-methylbenzyl)-1,2,4-oxadiazole-5-carboxamide

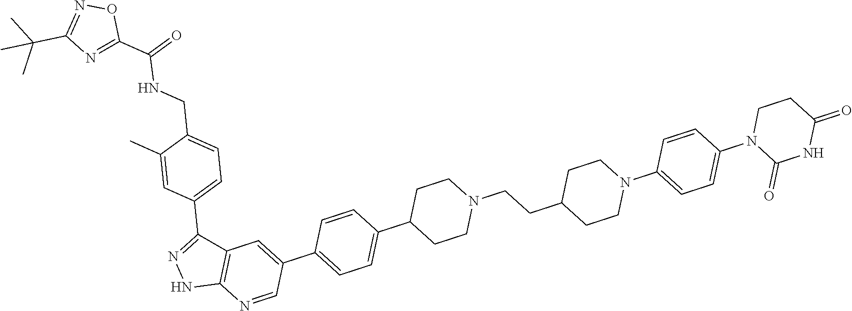 [SMILES: Cc1cc(-c2n[nH]c3ncc(-c4ccc(C5CCN(CCC6CCN(c7ccc(N8CCC(=O)NC8=O)cc7)CC6)CC5)cc4)cc23)ccc1CNC(=O)c1nc(C(C)(C)C)no1]

Step 1: A mixture of 3-(tert-butyl)-N-(2-methyl-4-(5-(4-(piperidin-4-yl)phenyl)-1H-pyrazolo[3,4-b]pyridin-3-yl)benzyl)-1,2,4-oxadiazole-5-carboxamide (192 mg, 0.35 mmol) and 2-(1-(4-(2,4-dioxotetrahydropyrimidin-1(2H)-yl)phenyl)piperidin-4-yl)acetaldehyde (100 mg, 0.32 mmol) in dichloromethane (20 mL) was stirred in a round bottom flask at room temperature for 1 hour. Then the mixture was added NaBH₃CN (40 mg, 0.63 mmol) and stirred in a round bottom flask at room temperature overnight. The mixture was evaporated in vacuum to afford the crude product, which was purified with silica gel column chromatography (DCM:MeOH=100:0˜80:20 gradient elution) to give the product (31.3 mg, 12%). ¹H NMR (400 MHz, DMSO) δ_{H} 13.92 (s, 1H), 10.33 (s, 1H), 9.92 (s, 1H), 8.90 (s, 1H), 8.72 (s, 1H), 8.02-7.91 (m, 2H), 7.81 (d, J=7.2 Hz, 2H), 7.51-7.39 (m, 3H), 7.19 (d, J=8.0 Hz, 2H), 6.98 (d, J=8.0 Hz, 2H), 4.58 (d, J=4.8 Hz, 2H), 3.80-3.70 (m, 4H), 3.25-3.15 (m, 2H), 2.77-2.66 (m, 5H), 2.51 (s, 3H), 2.40-2.20 (m, 2H), 1.94-1.75 (m, 6H), 1.55 (s, 3H), 1.47-1.38 (m, 9H), 1.37-1.24 (m, 3H); [M+H]⁺=849.6.

### Example 6: 5-(tert-butyl)-N-(4-(5-(4-(1-(4-(2,4-dioxotetrahydropyrimidin-1(2H)-yl)phenethyl)piperidin-4-yl)phenyl)-1H-pyrazolo[3,4-b]pyridin-3-yl)-2-methylbenzyl)-1,2,4-oxadiazole-3-carboxamide

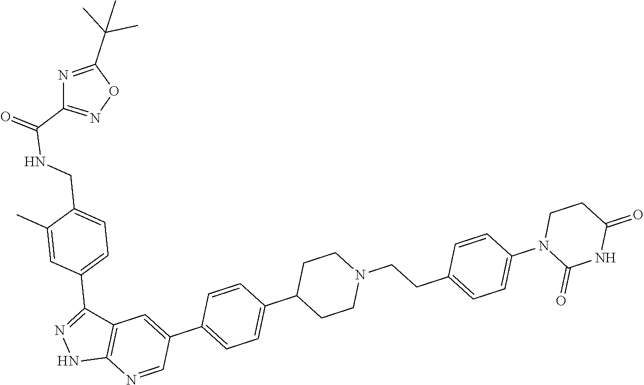 [SMILES: Cc1cc(-c2n[nH]c3ncc(-c4ccc(C5CCN(CCc6ccc(N7CCC(=O)NC7=O)cc6)CC5)cc4)cc23)ccc1CNC(=O)c1noc(C(C)(C)C)n1]

The titled compound was synthesized in the procedures similar to Example 1. ¹H NMR (400 MHz, DMSO) δ_{H} 13.84 (s, 1H), 10.35 (s, 1H), 9.46 (s, 1H), 8.85 (s, 1H), 8.66 (s, 1H), 7.90 (s, 2H), 7.74 (d, J=7.5 Hz, 2H), 7.40 (d, J=7.5 Hz, 3H), 7.25 (s, 4H), 4.52 (d, J=4.8 Hz, 2H), 3.77 (t, J=6.1 Hz, 2H), 3.09 (d, J=9.7 Hz, 2H), 2.77 (d, J=7.0 Hz, 2H), 2.70 (t, J=6.2 Hz, 2H), 2.56 (d, J=8.0 Hz, 3H), 2.45 (s, 3H), 2.11 (s, 2H), 1.85-1.68 (m, 4H), 1.44 (s, 9H); [M+H]⁺=766.6.

### Example 7: 3-(tert-butyl)-N-(4-(5-(4-(1-(3-(4-((2,6-dioxopiperidin-3-yl)amino)phenoxy)propyl)piperidin-4-yl)phenyl)-1H-pyrazolo[3,4-b]pyridin-3-yl)-2-methylbenzyl)-1,2,4-oxadiazole-5-carboxamide

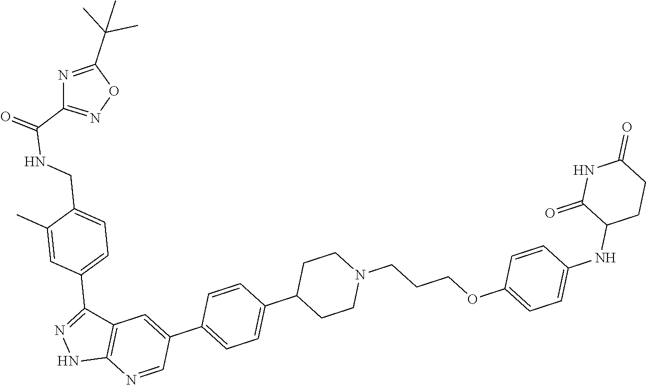 [SMILES: Cc1cc(-c2n[nH]c3ncc(-c4ccc(C5CCN(CCCOc6ccc(NC7CCC(=O)NC7=O)cc6)CC5)cc4)cc23)ccc1CNC(=O)c1noc(C(C)(C)C)n1]

To a solution of 3-(tert-butyl)-N-(2-methyl-4-(5-(4-(piperidin-4-yl)phenyl)-1H-pyrazolo[3,4-b]pyridin-3-yl)benzyl)-1,2,4-oxadiazole-5-carboxamide (25 mg, 0.04 mmol) and 3-(4-((2,6-dioxopiperidin-3-yl)amino)phenoxy)propyl methanesulfonate (23 mg, 0.06 mmol) in DMSO (2 mL) was added DIEA (17 mg, 0.12 mmol). The resulting mixture was heated at 90° C. overnight. The reaction was purified with pre-HPLC to give the titled product (1.66 mg, 4.5%). ¹H NMR (400 MHz, DMSO) δ_{H} 13.85 (s, 1H), 10.77 (s, 1H), 9.86 (s, 1H), 8.85 (s, 1H), 8.66 (s, 1H), 7.90 (s, 2H), 7.76 (d, J=7.7 Hz, 2H), 7.47-7.34 (m, 3H), 6.74 (d, J=8.7 Hz, 2H), 6.64 (d, J=9.0 Hz, 2H), 5.46 (d, J=7.1 Hz, 1H), 4.53 (d, J=5.1 Hz, 2H), 4.21 (s, 1H), 3.92 (s, 2H), 3.16 (s, 2H), 2.75-2.58 (m, 5H), 2.45 (s, 3H), 2.33 (s, 1H), 2.10 (s, 1H), 2.00-1.75 (m, 8H), 1.37 (s, 9H), 1.23 (s, 3H); [M+H]⁺=810.5.

### Example 8: 3-(tert-butyl)-N-(4-(5-(4-(2-(4-(4-(2,6-dioxopiperidin-3-yl)phenyl)piperidin-1-yl)ethyl)phenyl)-1H-pyrazolol[3,4-b]pyridin-3-yl)-2-methylbenzyl)-1,2,4-oxadiazole-5-carboxamide

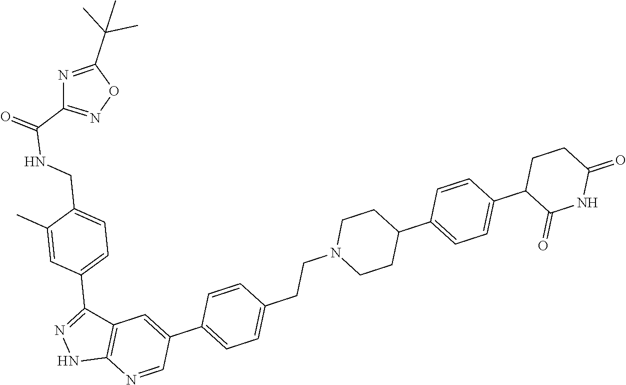 [SMILES: Cc1cc(-c2n[nH]c3ncc(-c4ccc(CCN5CCC(c6ccc(C7CCC(=O)NC7=O)cc6)CC5)cc4)cc23)ccc1CNC(=O)c1noc(C(C)(C)C)n1]

### Step 1: tert-butyl 5-bromo-3-(4-((3-(tert-butyl)-1,2,4-oxadiazole-5-carboxamido)methyl)-3-methylphenyl)-1H-pyrazolo[3,4-b]pyridine-1-carboxylate

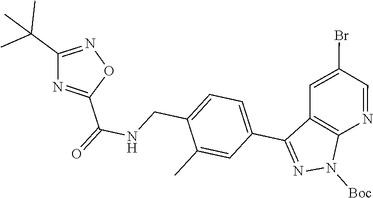 [SMILES: Cc1cc(-c2nn(C(=O)OC(C)(C)C)c3ncc(Br)cc23)ccc1CNC(=O)c1nc(C(C)(C)C)no1]

A mixture of tert-butyl 5-bromo-3-iodo-1H-pyrazolo[3,4-b]pyridine-1-carboxylate (190 mg, 0.45 mmol), 3-(tert-butyl)-N-(2-methyl-4-(4,4,5,5-tetramethyl-1,3,2-dioxaborolan-2-yl)benzyl)-1,2,4-oxadiazole-5-carboxamide (180 mg, 0.45 mmol), Pd(dppf)Cl₂ (33 mg, 0.045 mmol) and Cs₂CO₃ (219 mg, 0.67 mmol) in dioxane (10 mL)/H₂O (2 mL) was stirred in a round bottom flask at 80° C. for 1 h under N₂. The solvent was removed under reduced pressure and the crude product was purified with silica gel column chromatography (PE:EtOAc=10:1˜4:1 gradient elution) to give the product (120 mg, 47%). [M+H]⁺=569.2.

### Step 2: tert-butyl 3-(4-((3-(tert-butyl)-1,2,4-oxadiazole-5-carboxamido)methyl)-3-methylphenyl)-5-(4-(2-hydroxyethyl)phenyl)-1H-pyrazolo[3,4-b]pyridine-1-carboxylate

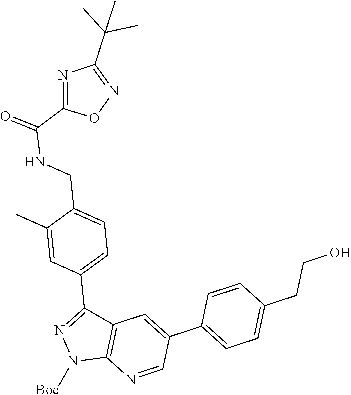 [SMILES: Cc1cc(-c2nn(C(=O)OC(C)(C)C)c3ncc(-c4ccc(CCO)cc4)cc23)ccc1CNC(=O)c1nc(C(C)(C)C)no1]

A mixture of tert-butyl 5-bromo-3-(4-((3-(tert-butyl)-1,2,4-oxadiazole-5-carboxamido)methyl)-3-methylphenyl)-1H-pyrazolo[3,4-b]pyridine-1-carboxylate (120 mg, 0.21 mmol), 2-(4-(4,4,5,5-tetramethyl-1,3,2-dioxaborolan-2-yl)phenyl)ethan-1-ol (53 mg, 0.21 mmol), Pd(dppf)Cl₂ (33 mg, 0.045 mmol) and Cs₂CO₃ (219 mg, 0.67 mmol) in dioxane (7.5 mL)/H₂O (1.5 mL) was stirred in a round bottom flask at 90° C. for 3 h under N₂. The solvent was removed under reduced pressure and the crude product was purified with silica gel column chromatography (DCM:MeOH=100:1˜10:1 gradient elution) to give the product (80 mg, 62%). [M+H]⁺=611.2.

### Step 3: 3-(tert-butyl)-N-(4-(5-(4-(2-hydroxyethyl)phenyl)-1H-pyrazolo[3,4-b]pyridin-3-yl)-2-methylbenzyl)-1,2,4-oxadiazole-5-carboxamide

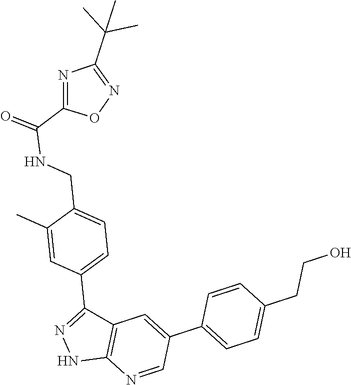 [SMILES: Cc1cc(-c2n[nH]c3ncc(-c4ccc(CCO)cc4)cc23)ccc1CNC(=O)c1nc(C(C)(C)C)no1]

A solution of tert-butyl 3-(4-((3-(tert-butyl)-1,2,4-oxadiazole-5-carboxamido)methyl)-3-methylphenyl)-5-(4-(2-hydroxyethyl)phenyl)-1H-pyrazolo[3,4-b]pyridine-1-carboxylate (80 mg, 0.13 mmol) in HCl/dioxane (5 mL) was stirred at room temperature for 2 hours. After the reaction was completed, the solvent was removed under reduced pressure. The residue was basified with sat. NaHCO₃ and extracted with DCM (2 *25 mL). The combined organic layers were dried over Na₂SO₄, filtered and concentrated to give the desired product (60 mg, 89%). [M+H]⁺=511.2.

### Step 4: 3-(tert-butyl)-N-(2-methyl-4-(5-(4-(2-oxoethyl)phenyl)-1H-pyrazolo[3,4-b]pyridin-3-yl)benzyl)-1,2,4-oxadiazole-5-carboxamide

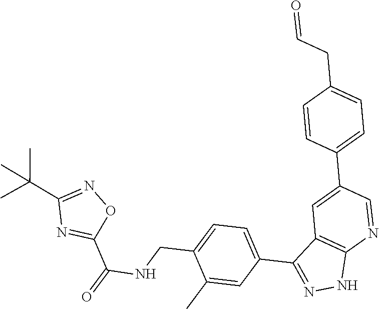 [SMILES: Cc1cc(-c2n[nH]c3ncc(-c4ccc(CC=O)cc4)cc23)ccc1CNC(=O)c1nc(C(C)(C)C)no1]

To a solution of 3-(tert-butyl)-N-(4-(5-(4-(2-hydroxyethyl)phenyl)-1H-pyrazolo[3,4-b]pyridin-3-yl)-2-methylbenzyl)-1,2,4-oxadiazole-5-carboxamide (125 mg, 0.24 mmol) in DMSO (4 mL) was added IBX (113 mg, 0.48 mmol). The resulting mixture was stirred at room temperature overnight. The reaction was quenched with water and extracted with EtOAc (2*30 mL). The combined organic layers were washed with brine, dried over anhydrous Na₂SO₄, and evaporated in vacuum to afford the desired product (110 mg, 88%). [M+H]⁺=509.2.

### Step 5: 3-(tert-butyl)-N-(4-(5-(4-(2-(4-(4-(2,6-dioxopiperidin-3-yl)phenyl)piperidin-1-yl)ethyl)phenyl)-1H-pyrazolo[3,4-b]pyridin-3-yl)-2-methylbenzyl)-1,2,4-oxadiazole-5-carboxamide

To a solution of 3-(tert-butyl)-N-(2-methyl-4-(5-(4-(2-oxoethyl)phenyl)-1H-pyrazolo[3,4-b]pyridin-3-yl)benzyl)-1,2,4-oxadiazole-5-carboxamide (50 mg, 0.1 mmol), 3-(4-(piperidin-4-yl)phenyl)piperidine-2,6-dione (30 mg, 0.1 mmol) and DIEA (15 mg, 0.1 mmol) in DCM (5 mL)/MeOH (1 mL) was added acetic acid (6 mg, 0.1 mmol). After stirring at room temperature for 0.5 h, NaBH(OAc)₃ (65 mg, 0.3 mmol) was added and the resulting mixture was stirred at room temperature for 2 h. The reaction was quenched with water and extracted with DCM. The organic layer was dried over anhydrous Na₂SO₄, and evaporated in vacuum to afford the crude product, which was further purified with pre-HPLC to give the product (6.2 mg, 8%). ¹H NMR (400 MHz, DMSO) δ_{H} 13.86 (s, 1H), 10.83 (s, 1H), 9.87 (s, 1H), 8.87 (s, 1H), 8.67 (s, 1H), 7.92 (d, J=10.8 Hz, 2H), 7.78 (d, J=7.3 Hz, 2H), 7.42 (t, J=7.3 Hz, 3H), 7.19 (dd, J=26.4, 7.7 Hz, 4H), 4.53 (d, J=5.3 Hz, 2H), 3.82 (d, J=7.0 Hz, 1H), 3.30-3.19 (m, 2H), 3.02-2.82 (m, 4H), 2.65 (d, J=11.9 Hz, 3H), 2.46 (s, 5H), 2.18 (d, J=10.4 Hz, 1H), 2.04 (s, 1H), 1.81 (d, J=31.9 Hz, 4H), 1.37 (s, 9H); [M+H]⁺=765.5.

### Example 9: 3-(tert-butyl)-N-(4-(5-(4-(1-(4-(2,6-dioxopiperidin-3-yl)phenethyl)piperidin-4-yl)phenyl)-1H-pyrazolo[3,4-b]pyridin-3-yl)-2-methylbenzyl)-1,2,4-oxadiazole-5-carboxamide

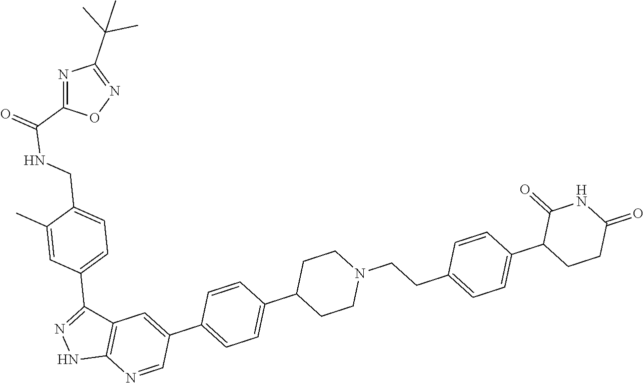 [SMILES: Cc1cc(-c2n[nH]c3ncc(-c4ccc(C5CCN(CCc6ccc(C7CCC(=O)NC7=O)cc6)CC5)cc4)cc23)ccc1CNC(=O)c1nc(C(C)(C)C)no1]

The titled compound was synthesized in the procedures similar to Example 4. ¹H NMR (400 MHz, DMSO) δ_{H} 13.85 (s, 1H), 10.83 (s, 1H), 9.86 (s, 1H), 8.85 (s, 1H), 8.66 (s, 1H), 8.23 (s, 1H), 7.92 (d, J=11.1 Hz, 2H), 7.74 (d, J=8.0 Hz, 2H), 7.41 (t, J=8.8 Hz, 3H), 7.21 (d, J=8.4 Hz, 2H), 7.14 (d, J=8.0 Hz, 2H), 4.53 (d, J=5.3 Hz, 2H), 3.83 (s, 1H), 3.09 (d, J=11.7 Hz, 3H), 2.76 (s, 2H), 2.67 (s, 1H), 2.55 (d, J=9.3 Hz, 3H), 2.45 (s, 3H), 2.33 (s, 1H), 2.19-2.07 (m, 3H), 1.83-1.70 (m, 4H), 1.37 (s, 9H); [M+H]⁺=765.4.

### Example 10: 3-(tert-butyl)-N-(4-(5-(4-(1-((1-(4-(2,4-dioxotetrahydropyrimidin-1(2H)-yl)phenyl)piperidin-4-yl)methyl)piperidin-4-yl)phenyl)-1H-pyrazolo[3,4-b]pyridin-3-yl)-2-fluorobenzyl)-1,2,4-oxadiazole-5-carboxamide

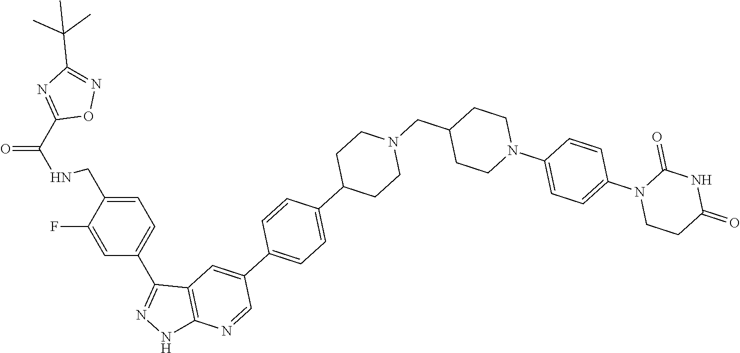 [SMILES: CC(C)(C)c1noc(C(=O)NCc2ccc(-c3n[nH]c4ncc(-c5ccc(C6CCN(CC7CCN(c8ccc(N9CCC(=O)NC9=O)cc8)CC7)CC6)cc5)cc34)cc2F)n1]

### Step 1: N-(4-(5-bromo-1-(tetrahydro-2H-pyran-2-yl)-1H-pyrazolo[3,4-b]pyridin-3-yl)-2-fluorobenzyl)-3-(tert-butyl)-1,2,4-oxadiazole-5-carboxamide

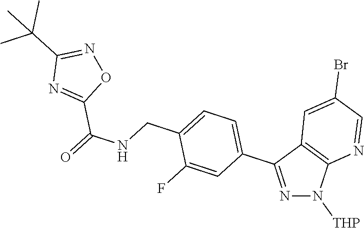

A mixture of 5-bromo-3-iodo-1-(tetrahydro-2H-pyran-2-yl)-1H-pyrazolo[3,4-b]pyridine (3 g, 7.35 mmol), 3-(tert-butyl)-N-(2-fluoro-4-(4,4,5,5-tetramethyl-1,3,2-dioxaborolan-2-yl)benzyl)-1,2,4-oxadiazole-5-carboxamide (2.96 g, 7.35 mmol), K₂CO₃ (2.54 g, 18.38 mmol) and Pd(dppf)Cl₂ (0.27 g, 0.368 mmol) in dioxane (100 mL) and H₂O (10 mL) was stirred in a sealed tube at 80° C. overnight. After cooling, the reaction was quenched with water and the mixture was extracted with EtOAc. The organic layer was dried over anhydrous Na₂SO₄, and evaporated in vacuum to afford the crude product, which was further purified with silica gel column chromatography (EtOAc in PE from 0% to 40% gradient elution) to give the product (3.2 g, 78.1%). [M+H]⁺=557.0.

### Step 2: tert-butyl 4-(4-(3-(4-((3-(tert-butyl)-1,2,4-oxadiazole-5-carboxamido)methyl)-3-fluorophenyl)-1-(tetrahydro-2H-pyran-2-yl)-1H-pyrazolo[3,4-b]pyridin-5-yl)phenyl)piperidine-1-carboxylate

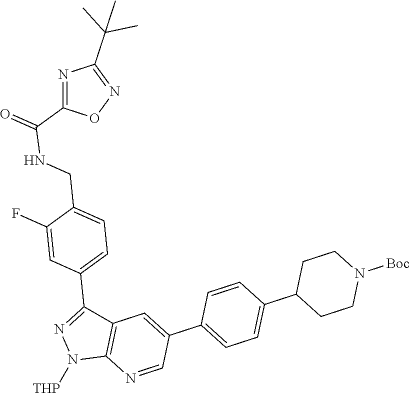

A mixture of N-(4-(5-bromo-1-(tetrahydro-2H-pyran-2-yl)-1H-pyrazolo[3,4-b]pyridin-3-yl)-2-fluorobenzyl)-3-(tert-butyl)-1,2,4-oxadiazole-5-carboxamide (2 g, 3.59 mmol), tert-butyl 4-(4-(4,4,5,5-tetramethyl-1,3,2-dioxaborolan-2-yl)phenyl)piperidine-1-carboxylate (1.53 g, 3.95 mmol), K₂CO₃ (1.24 g, 8.97 mmol) and Pd(dppf)Cl₂ (0.131 g, 0.1795 mmol) in dioxane (50 mL) and H₂O (5 mL) was stirred in a sealed tube at 100° C. overnight. After cooling, the reaction was quenched with water and the mixture was extracted with EtOAc. The organic layer was dried over anhydrous Na₂SO₄, and evaporated in vacuum to afford the crude product, which was further purified with silica gel column chromatography (EtOAc in PE from 0% to 40% gradient elution) to give the product (2.41 g, 91.0%). [M+H]⁺=738.0.

### Step 3: 3-(tert-butyl)-N-(2-fluoro-4-(5-(4-(piperidin-4-yl)phenyl)-1H-pyrazolo[3,4-b]pyridin-3-yl)benzyl)-1,2,4-oxadiazole-5-carboxamide hydrochloride

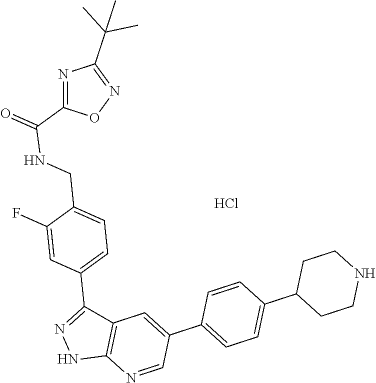 [SMILES: CC(C)(C)c1noc(C(=O)NCc2ccc(-c3n[nH]c4ncc(-c5ccc(C6CCNCC6)cc5)cc34)cc2F)n1 ; Cl]

To a solution of tert-butyl 4-(4-(3-(4-((3-(tert-butyl)-1,2,4-oxadiazole-5-carboxamido)methyl)-3-fluorophenyl)-1-(tetrahydro-2H-pyran-2-yl)-1H-pyrazolo[3,4-b]pyridin-5-yl)phenyl)piperidine-1-carboxylate (0.65 g, 0.88 mmol) in DCM (20 mL) in a round bottom flask was added HCl in dioxane (4N, 20 mL) at 0° C. The mixture was stirred for 2 h at 20° C. The precipitate was collected with filtration and dried in vacuum to afford the product (0.52 g, 100%). ¹H NMR (400 MHz, DMSO) δ_{H} 9.96 (s, 1H), 8.94 (s, 1H), 8.89 (s, 1H), 8.83 (s, 1H), 8.72 (s, 1H), 7.97 (d, J=8.0 Hz, 1H), 7.89 (d, J=10.9 Hz, 1H), 7.83 (d, J=7.7 Hz, 2H), 7.56 (t, J=7.8 Hz, 1H), 7.38 (d, J=7.9 Hz, 2H), 4.58 (d, J=5.6 Hz, 2H), 3.57 (s, 1H), 3.38 (d, J=12.2 Hz, 2H), 2.98 (dd, J=27.4, 14.3 Hz, 3H), 2.08-1.81 (m, 4H), 1.37 (s, 9H); [M+H]⁺=554.6.

### Step 4: 3-(tert-butyl)-N-(4-(5-(4-(1-((1-(4-(2,4-dioxotetrahydropyrimidin-1(2H)-yl)phenyl)piperidin-4-yl)methyl)piperidin-4-yl)phenyl)-1H-pyrazolo[3,4-b]pyridin-3-yl)-2-fluorobenzyl)-1,2,4-oxadiazole-5-carboxamide

A mixture of 3-(tert-butyl)-N-(2-fluoro-4-(5-(4-(piperidin-4-yl)phenyl)-1H-pyrazolo[3,4-b]pyridin-3-yl)benzyl)-1,2,4-oxadiazole-5-carboxamide hydrochloride (0.5 g, 0.847 mmol), 1-(4-(2,4-dioxotetrahydropyrimidin-1(2H)-yl)phenyl)piperidine-4-carbaldehyde (0.28 g, 0.932 mmol) and NaOAc (70 mg, 0.847 mmol) in DCM/EtOH (100 mL/30 mL) was stirred in a round bottom flask for 1 h at 20° C. Then NaBH₃CN (0.107 g, 1.69 mmol) was added. The mixture was stirred for 3 h at 20° C. The mixture was concentrated to dryness and purified with silica gel column chromatography (MeOH in DCM from 0% to 12% gradient elution) to give the product (0.48 g, 67.5%). ¹H NMR (400 MHz, DMSO) δ_{H} 14.14 (s, 1H), 12.09 (s, 1H), 10.40 (s, 1H), 10.07 (s, 1H), 9.01 (s, 1H), 8.85 (s, 1H), 8.09 (d, J=7.7 Hz, 1H), 8.02 (d, J=11.2 Hz, 1H), 7.94 (s, 2H), 7.69 (t, J=7.8 Hz, 1H), 7.53 (d, J=8.0 Hz, 2H), 7.27 (d, J=8.3 Hz, 2H), 7.08 (d, J=8.0 Hz, 2H), 4.71 (d, J=5.2 Hz, 2H), 3.82 (t, J=6.6 Hz, 4H), 3.46 (s, 6H), 3.29 (d, J=4.9 Hz, 1H), 2.88-2.76 (m, 4H), 2.16 (s, 2H), 2.03 (s, 2H), 1.97 (d, J=10.4 Hz, 2H), 1.88 (s, 1H), 1.49 (s, 9H); [M+H]⁺=839.5.

### Example 11: 3-(tert-butyl)-N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-fluorobenzyl)-1,2,4-oxadiazole-5-carboxamide

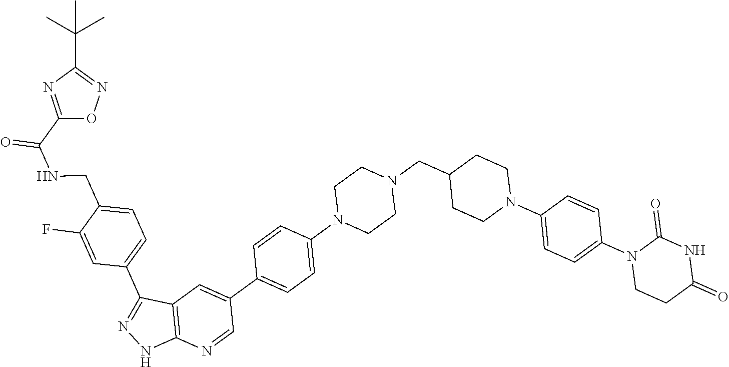 [SMILES: CC(C)(C)c1noc(C(=O)NCc2ccc(-c3n[nH]c4ncc(-c5ccc(N6CCN(CC7CCN(c8ccc(N9CCC(=O)NC9=O)cc8)CC7)CC6)cc5)cc34)cc2F)n1]

### Step 1: N-(4-(5-bromo-1-(tetrahydro-2H-pyran-2-yl)-1H-pyrazolo[3,4-b]pyridin-3-yl)-2-fluorobenzyl)-3-(tert-butyl)-1,2,4-oxadiazole-5-carboxamide

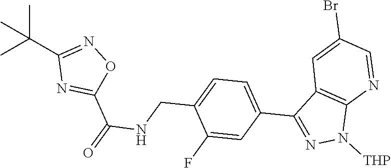

A mixture of 5-bromo-3-iodo-1-(tetrahydro-2H-pyran-2-yl)-1H-pyrazolo[3,4-b]pyridine (3 g, 7.35 mmol), 3-(tert-butyl)-N-(2-fluoro-4-(4,4,5,5-tetramethyl-1,3,2-dioxaborolan-2-yl)benzyl)-1,2,4-oxadiazole-5-carboxamide (2.96 g, 7.35 mmol), K₂CO₃ (2.54 g, 18.38 mmol) and Pd(dppf)Cl₂ (0.27 g, 0.368 mmol) in dioxane (100 mL) and H₂O (10 mL) was stirred in a sealed tube at 80° C. overnight. After cooling, the reaction was quenched with water and the mixture was extracted with EtOAc. The organic layer was dried over anhydrous Na₂SO₄, and evaporated in vacuum to afford the crude product, which was further purified with silica gel column chromatography (EtOAc in PE from 0% to 40% gradient elution) to give the product (3.2 g, 78.1%). [M+H]⁺=557.0.

### Step 2: tert-butyl 4-(4-(3-(4-((3-(tert-butyl)-1,2,4-oxadiazole-5-carboxamido)methyl)-3-fluorophenyl)-1-(tetrahydro-2H-pyran-2-yl)-1H-pyrazolo[3,4-b]pyridin-5-yl)phenyl)piperazine-1-carboxylate

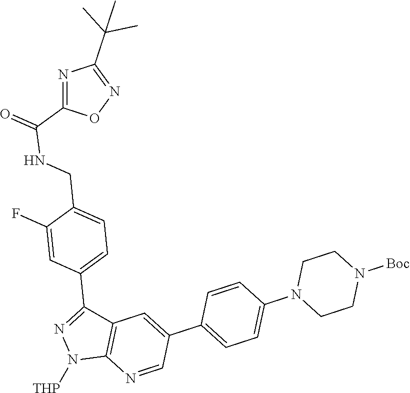

A mixture of N-(4-(5-bromo-1-(tetrahydro-2H-pyran-2-yl)-1H-pyrazolo[3,4-b]pyridin-3-yl)-2-fluorobenzyl)-3-(tert-butyl)-1,2,4-oxadiazole-5-carboxamide (0.9 g, 1.615 mmol), tert-butyl 4-(4-(4,4,5,5-tetramethyl-1,3,2-dioxaborolan-2-yl)phenyl)piperazine-1-carboxylate (0.69 g, 1.78 mmol), K₂CO₃ (0.557 g, 4.04 mmol) and Pd(dppf)Cl₂ (0.06 g, 0.08 mmol) in dioxane (40 mL) and H₂O (4 mL) was stirred in a sealed tube at 100° C. for 4 hours. After cooling, the reaction was quenched with water and the mixture was extracted with EtOAc. The organic layer was dried over anhydrous Na₂SO₄, and evaporated in vacuum to afford the crude product, which was further purified with silica gel column chromatography (EtOAc in PE from 0% to 30% gradient elution) to give the product (0.65 g, 54.4%). [M+H]⁺=739.5.

### Step 3: 3-(tert-butyl)-N-(2-fluoro-4-(5-(4-(piperazin-1-yl)phenyl)-1H-pyrazolo[3,4-b]pyridin-3-yl)benzyl)-1,2,4-oxadiazole-5-carboxamide bi-hydrochloride

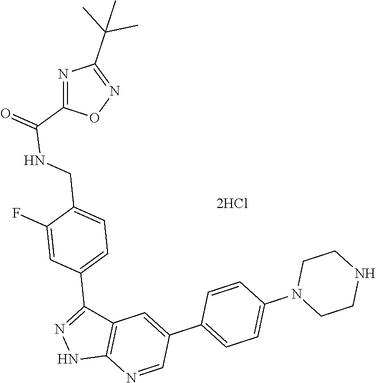 [SMILES: CC(C)(C)c1noc(C(=O)NCc2ccc(-c3n[nH]c4ncc(-c5ccc(N6CCNCC6)cc5)cc34)cc2F)n1 ; "2HCl"]

To a solution of tert-butyl 4-(4-(3-(4-((3-(tert-butyl)-1,2,4-oxadiazole-5-carboxamido)methyl)-3-fluorophenyl)-1-(tetrahydro-2H-pyran-2-yl)-1H-pyrazolo[3,4-b]pyridin-5-yl)phenyl)piperazine-1-carboxylate (0.5 g, 0.68 mmol) in DCM (10 mL) in a round bottom flask was added HCl in dioxane (4N, 20 mL) at 0° C. The mixture was stirred for 2 h at 20° C. The precipitate was collected with filtration and dried in vacuum to afford the product (0.40 g, 94.2%). ¹H NMR (400 MHz, DMSO) δ_{H} 9.95 (s, 1H), 9.20 (s, 2H), 8.87 (s, 1H), 8.66 (s, 1H), 7.96 (d, J=7.8 Hz, 1H), 7.88 (d, J=11.2 Hz, 1H), 7.78 (d, J=8.1 Hz, 2H), 7.56 (t, J=8.2 Hz, 1H), 7.13 (d, J=8.5 Hz, 2H), 7.07-6.44 (m, 2H), 4.58 (d, J=5.3 Hz, 2H), 3.57 (s, 4H), 3.45 (s, 4H), 3.25 (s, 4H), 1.37 (s, 9H); [M+H]⁺=555.6.

### Step 4: 3-(tert-butyl)-N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-fluorobenzyl)-1,2,4-oxadiazole-5-carboxamide

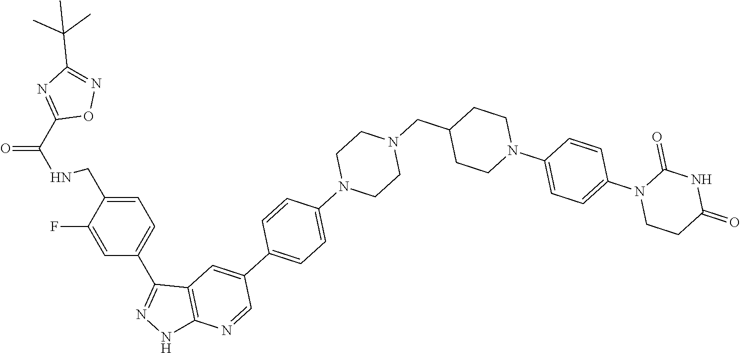 [SMILES: CC(C)(C)c1noc(C(=O)NCc2ccc(-c3n[nH]c4ncc(-c5ccc(N6CCN(CC7CCN(c8ccc(N9CCC(=O)NC9=O)cc8)CC7)CC6)cc5)cc34)cc2F)n1]

A mixture of 3-(tert-butyl)-N-(2-fluoro-4-(5-(4-(piperazin-1-yl)phenyl)-1H-pyrazolo[3,4-b]pyridin-3-yl)benzyl)-1,2,4-oxadiazole-5-carboxamide bi-hydrochloride (0.2 g, 0.319 mmol), 1-(4-(2,4-dioxotetrahydropyrimidin-1(2H)-yl)phenyl)piperidine-4-carbaldehyde (0.106 g, 0.351 mmol) and NaOAc (52.3 mg, 0.637 mmol) in DCM/EtOH (60 mL/20 mL) was stirred in a round bottom flask for 1 h at 20° C. Then NaBH₃CN (40.1 mg, 0.637 mmol) was added. The mixture was stirred at 20° C. overnight. The mixture was concentrated to dryness and purified with silica gel column chromatography (MeOH in DCM from 0% to 12% gradient elution) to give the product (64.8 mg, 24.2%). ¹H NMR (400 MHz, DMSO) δ_{H} 13.93 (s, 1H), 10.26 (s, 1H), 9.94 (s, 1H), 8.85 (s, 1H), 8.63 (s, 1H), 7.96 (d, J=8.1 Hz, 1H), 7.87 (d, J=10.9 Hz, 1H), 7.71 (d, J=8.1 Hz, 2H), 7.56 (s, 1H), 7.13 (d, J=8.1 Hz, 3H), 7.07 (d, J=8.2 Hz, 2H), 6.93 (d, J=7.8 Hz, 3H), 4.58 (d, J=5.4 Hz, 2H), 3.68 (d, J=7.1 Hz, 1H), 3.27 (d, J=5.4 Hz, 1H), 3.22 (s, 4H), 2.68 (d, J=7.7 Hz, 1H), 2.24 (d, J=6.8 Hz, 2H), 1.82 (d, J=12.3 Hz, 2H), 1.73 (d, J=11.9 Hz, 2H), 1.37 (s, 9H), 1.23 (d, J=11.9 Hz, 2H); [M+H]⁺=840.5.

### Example 12: 3-(tert-butyl)-N-(4-(5-(4-(1-(2-(1-(4-(2,4-dioxotetrahydropyrimidin-1(2H)-yl)phenyl)piperidin-4-yl)ethyl)piperidin-4-yl)phenyl)-1H-pyrazolo[3,4-b]pyridin-3-yl)-2-fluorobenzyl)-1,2,4-oxadiazole-5-carboxamide

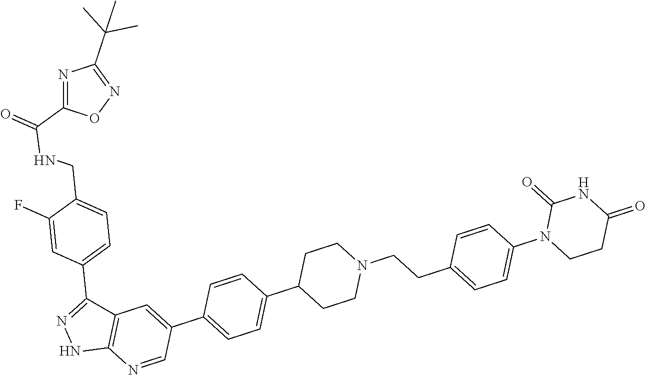 [SMILES: CC(C)(C)c1noc(C(=O)NCc2ccc(-c3n[nH]c4ncc(-c5ccc(C6CCN(CCc7ccc(N8CCC(=O)NC8=O)cc7)CC6)cc5)cc34)cc2F)n1]

The titled compound was synthesized in the procedures similar to Example 10. ¹H NMR (400 MHz, DMSO) δ_{H} 14.02 (s, 1H), 10.28 (s, 1H), 9.95 (s, 1H), 8.89 (s, 1H), 8.72 (s, 1H), 7.96 (s, 1H), 7.93-7.74 (m, 3H), 7.56 (s, 1H), 7.40 (s, 2H), 7.13 (s, 2H), 6.95 (s, 2H), 4.59 (s, 2H), 3.69 (s, 5H), 3.24-3.15 (m, 1H), 3.13-2.90 (m, 4H), 2.68 (s, 4H), 2.08 (s, 2H), 1.91 (s, 2H), 1.78 (s, 3H), 1.73-1.60 (m, 2H), 1.49 (s, 2H), 1.37 (s, 9H), 1.34-1.25 (m, 1H); [M+H]⁺=853.6.

### Example 13: 3-(tert-butyl)-N-(4-(5-(4-(1-(4-(2,4-dioxotetrahydropyrimidin-1(2H)-yl)phenethyl)piperidin-4-yl)phenyl)-1H-pyrazolo[3,4-b]pyridin-3-yl)-2-fluorobenzyl)-1,2,4-oxadiazole-5-carboxamide

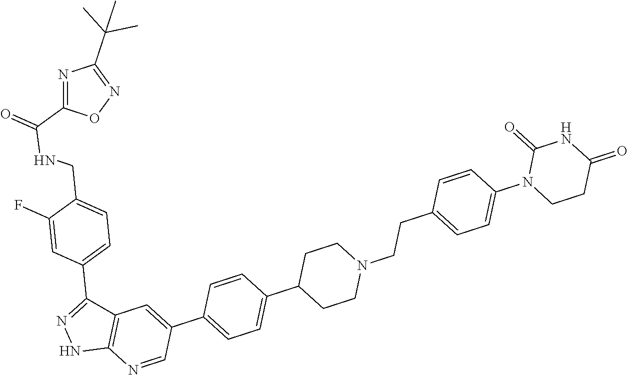 [SMILES: CC(C)(C)c1noc(C(=O)NCc2ccc(-c3n[nH]c4ncc(-c5ccc(C6CCN(CCc7ccc(N8CCC(=O)NC8=O)cc7)CC6)cc5)cc34)cc2F)n1]

The titled compound was synthesized in the procedures similar to Example 10. ¹H NMR (400 MHz, DMSO) δ_{H} 14.01 (s, 1H), 10.37 (s, 1H), 9.95 (s, 1H), 8.88 (s, 1H), 8.72 (s, 1H), 7.97 (d, J=8.0 Hz, 1H), 7.89 (d, J=11.3 Hz, 1H), 7.80 (d, J=7.7 Hz, 2H), 7.56 (t, J=7.9 Hz, 1H), 7.41 (d, J=7.6 Hz, 2H), 7.29 (s, 4H), 4.58 (d, J=5.4 Hz, 2H), 3.77 (t, J=6.5 Hz, 2H), 3.33 (s, 4H), 2.89 (s, 4H), 2.71 (t, J=6.4 Hz, 3H), 1.87 (d, J=32.4 Hz, 4H), 1.37 (s, 9H); [M+H]⁺=770.7.

### Example 14: 3-(tert-butyl)-N-(4-(5-(4-(1-(4-(2,6-dioxopiperidin-3-yl)phenethyl)piperidin-4-yl)phenyl)-1H-pyrazolo[3,4-b]pyridin-3-yl)-2-fluorobenzyl)-1,2,4-oxadiazole-5-carboxamide

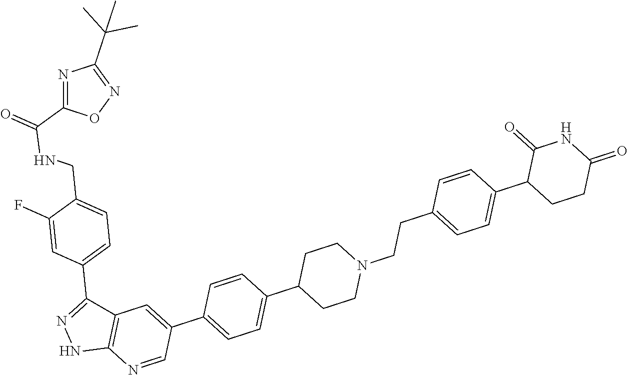 [SMILES: CC(C)(C)c1noc(C(=O)NCc2ccc(-c3n[nH]c4ncc(-c5ccc(C6CCN(CCc7ccc(C8CCC(=O)NC8=O)cc7)CC6)cc5)cc34)cc2F)n1]

The titled compound was synthesized in the procedures similar to Example 10. ¹H NMR (400 MHz, DMSO) δ_{H} 14.01 (s, 1H), 10.85 (s, 1H), 9.95 (s, 1H), 8.88 (s, 1H), 8.72 (s, 1H), 7.97 (d, J=8.0 Hz, 1H), 7.89 (d, J=11.2 Hz, 1H), 7.79 (d, J=7.8 Hz, 2H), 7.56 (t, J=7.9 Hz, 1H), 7.41 (d, J=7.6 Hz, 2H), 7.24 (d, J=7.5 Hz, 2H), 7.17 (d, J=7.2 Hz, 2H), 4.59 (d, J=5.6 Hz, 2H), 3.88-3.79 (m, 1H), 3.34 (s, 2H), 2.72 (dd, J=46.1, 34.0 Hz, 5H), 2.47 (s, 1H), 2.26-1.97 (m, 3H), 1.91 (s, 6H), 1.37 (s, 9H); [M+H]⁺=769.5.

### Example 15: 5-(tert-butyl)-N-(4-(5-(4-(1-((1-(4-(2,4-dioxotetrahydropyrimidin-1(2H)-yl)phenyl)piperidin-4-yl)methyl)piperidin-4-yl)phenyl)-1H-pyrazolo[3,4-b]pyridin-3-yl)-2-fluorobenzyl)-1,2,4-oxadiazole-3-carboxamide

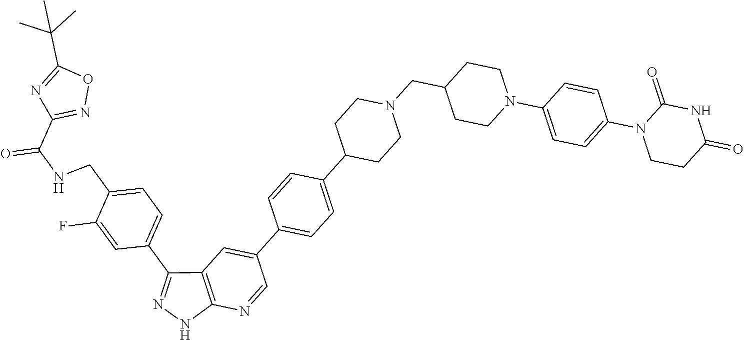 [SMILES: CC(C)(C)c1nc(C(=O)NCc2ccc(-c3n[nH]c4ncc(-c5ccc(C6CCN(CC7CCN(c8ccc(N9CCC(=O)NC9=O)cc8)CC7)CC6)cc5)cc34)cc2F)no1]

### Step 1: 5-(tert-butyl)-N-(2-fluoro-4-(5-(4-(piperidin-4-yl)phenyl)-1H-pyrazolo[3,4-b]pyridin-3-yl)benzyl)-1,2,4-oxadiazole-3-carboxamide hydrochloride

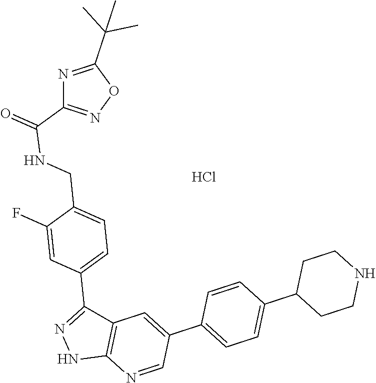 [SMILES: CC(C)(C)c1nc(C(=O)NCc2ccc(-c3n[nH]c4ncc(-c5ccc(C6CCNCC6)cc5)cc34)cc2F)no1 ; Cl]

To a solution of tert-butyl 4-(4-(3-(4-((5-(tert-butyl)-1,2,4-oxadiazole-3-carboxamido)methyl)-3-fluorophenyl)-1-(tetrahydro-2H-pyran-2-yl)-1H-pyrazolo[3,4-b]pyridin-5-yl)phenyl)piperidine-1-carboxylate (2 g, 2.71 mmol) in EtOAc (100 mL) in a round bottom flask was added HCl in dioxane (4N, 50 mL) at 0° C. The mixture was stirred for 3 h at 20° C. The precipitate was collected with filtration and dried in vacuum to afford the product (1.56 g, 97.6%). ¹H NMR (400 MHz, DMSO) δ_{H} 9.54 (s, 1H), 8.87 (s, 2H), 8.71 (s, 2H), 7.95 (d, J=6.7 Hz, 1H), 7.84 (dd, J=19.3, 9.0 Hz, 3H), 7.50 (s, 1H), 7.36 (d, J=6.0 Hz, 2H), 4.57 (s, 2H), 3.55 (s, 3H), 3.38 (d, J=11.6 Hz, 2H), 3.09-2.82 (m, 4H), 2.06-1.75 (m, 4H), 1.42 (s, 9H); [M+H]⁺=554.7.

### Step 2: 5-(tert-butyl)-N-(4-(5-(4-(1-((1-(4-(2,4-dioxotetrahydropyrimidin-1(2H)-yl)phenyl)piperidin-4-yl)methyl)piperidin-4-yl)phenyl)-1H-pyrazolo[3,4-b]pyridin-3-yl)-2-fluorobenzyl)-1,2,4-oxadiazole-3-carboxamide

A mixture of 5-(tert-butyl)-N-(2-fluoro-4-(5-(4-(piperidin-4-yl)phenyl)-1H-pyrazolo[3,4-b]pyridin-3-yl)benzyl)-1,2,4-oxadiazole-3-carboxamide hydrochloride (0.12 g, 0.847 mmol), 1-(4-(2,4-dioxotetrahydropyrimidin-1(2H)-yl)phenyl)piperidine-4-carbaldehyde (67.3 mg, 0.22 mmol) and NaOAc (33.3 mg, 0.4 mmol) in DCM/EtOH (30 mL/8 mL) was stirred in a round bottom flask for 1 h at 20° C. Then NaBH₃CN (25.6 mg, 0.4 mmol) was added. The mixture was stirred at 20° C. overnight. The mixture was concentrated to dryness and purified with silica gel column chromatography (MeOH in DCM from 0% to 12% gradient elution) to give the product (128 mg, 75.0%). ¹H NMR (400 MHz, DMSO) δ_{H} 14.13 (s, 1H), 10.41 (s, 1H), 9.69 (s, 1H), 9.20 (s, 1H), 9.01 (s, 1H), 8.85 (s, 1H), 8.09 (d, J=7.9 Hz, 1H), 8.04-7.88 (m, 3H), 7.64 (t, J=7.9 Hz, 1H), 7.52 (d, J=7.7 Hz, 2H), 7.28 (d, J=8.3 Hz, 2H), 7.08 (d, J=8.2 Hz, 2H), 4.70 (d, J=5.5 Hz, 2H), 3.84 (dd, J=17.3, 10.5 Hz, 6H), 3.46 (s, 3H), 3.20 (s, 4H), 2.81 (t, J=6.5 Hz, 4H), 2.16 (s, 4H), 2.06-1.93 (m, 3H), 1.56 (s, 9H), 1.47 (s, 2H); [M+H]⁺=839.8.

### Example 16: 3-(tert-butyl)-N-(4-(5-(4-(1-(2-(1-(4-(2,4-dioxotetrahydropyrimidin-1(2H)-yl)-3-fluorophenyl)piperidin-4-yl)ethyl)piperidin-4-yl)phenyl)-1H-pyrazolo[3,4-b]pyridin-3-yl)-2-fluorobenzyl)-1,2,4-oxadiazole-5-carboxamide

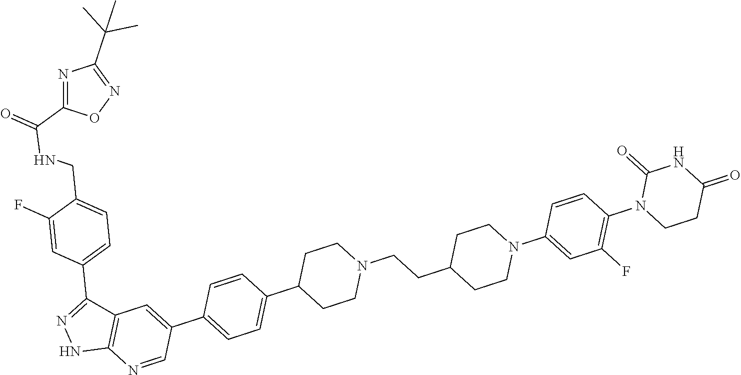 [SMILES: CC(C)(C)c1noc(C(=O)NCc2ccc(-c3n[nH]c4ncc(-c5ccc(C6CCN(CCC7CCN(c8ccc(N9CCC(=O)NC9=O)c(F)c8)CC7)CC6)cc5)cc34)cc2F)n1]

The titled compound was synthesized in the procedures similar to Example 10. ¹H NMR (400 MHz, DMSO) δ_{H} 14.03 (s, 1H), 10.40 (s, 1H), 9.96 (s, 1H), 8.89 (s, 1H), 8.72 (s, 1H), 7.96 (s, 1H), 7.89 (d, J=10.7 Hz, 1H), 7.82 (s, 2H), 7.56 (s, 1H), 7.40 (s, 2H), 7.20 (s, 1H), 6.88-6.74 (m, 2H), 4.59 (s, 2H), 3.78 (d, J=11.6 Hz, 2H), 3.62 (s, 2H), 3.34 (s, 4H), 2.73 (d, J=25.5 Hz, 6H), 1.95 (d, J=32.1 Hz, 4H), 1.77 (d, J=11.4 Hz, 2H), 1.63 (s, 2H), 1.52 (s, 1H), 1.37 (s, 9H), 1.26 (d, J=11.6 Hz, 3H); [M+H]⁺=871.5.

### Example 17: 3-(tert-butyl)-N-(4-(5-(4-(4-(2-(1-(4-(2,4-dioxotetrahydropyrimidin-1(2H)-yl)-3-fluorophenyl)piperidin-4-yl)ethyl)piperazin-1-yl)phenyl)-1H-pyrazolo[3,4-b]pyridin-3-yl)-2-fluorobenzyl)-1,2,4-oxadiazole-5-carboxamide

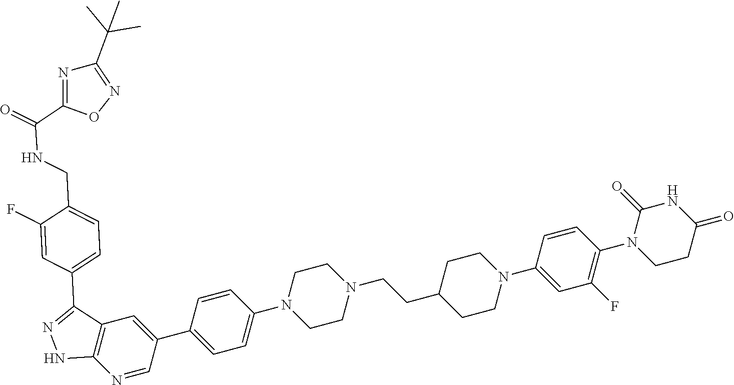 [SMILES: CC(C)(C)c1noc(C(=O)NCc2ccc(-c3n[nH]c4ncc(-c5ccc(N6CCN(CCC7CCN(c8ccc(N9CCC(=O)NC9=O)c(F)c8)CC7)CC6)cc5)cc34)cc2F)n1]

The titled compound was synthesized in the procedures similar to Example 11. ¹H NMR (400 MHz, DMSO) δ_{H} 13.95 (s, 1H), 10.40 (s, 1H), 9.95 (s, 1H), 8.85 (s, 1H), 8.63 (s, 1H), 7.96 (d, J=7.2 Hz, 1H), 7.87 (d, J=10.7 Hz, 1H), 7.71 (d, J=7.7 Hz, 2H), 7.56 (s, 1H), 7.17 (d, J=8.5 Hz, 1H), 7.06 (d, J=7.3 Hz, 2H), 6.78 (dd, J=21.9, 11.5 Hz, 2H), 4.59 (s, 2H), 3.74 (d, J=11.7 Hz, 2H), 3.62 (s, 2H), 3.35 (s, 2H), 3.21 (s, 4H), 2.70 (s, 4H), 2.54 (s, OH), 2.40 (s, 2H), 1.76 (d, J=12.8 Hz, 2H), 1.46 (s, 3H), 1.37 (s, 9H), 1.24 (s, 3H); [M+H]⁺=872.5.

### Example 18: 3-(tert-butyl)-N-(4-(5-(4-(1-(3-(2,4-dioxotetrahydropyrimidin-1(2H)-yl)-4-methoxybenzoyl)piperidin-4-yl)phenyl)-1H-pyrazolo[3,4-b]pyridin-3-yl)-2-fluorobenzyl)-1,2,4-oxadiazole-5-carboxamide

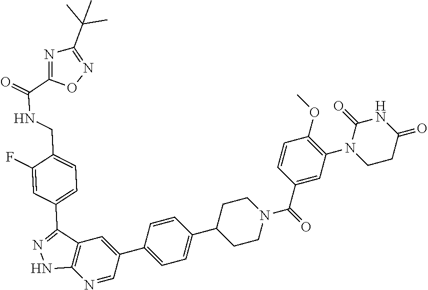 [SMILES: COc1ccc(C(=O)N2CCC(c3ccc(-c4cnc5[nH]nc(-c6ccc(CNC(=O)c7nc(C(C)(C)C)no7)c(F)c6)c5c4)cc3)CC2)cc1N1CCC(=O)NC1=O]

### Step 1: 3-(2,4-dioxotetrahydropyrimidin-1(2H-yl)₄-methoxybenzoic acid

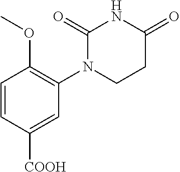 [SMILES: COc1ccc(C(=O)O)cc1N1CCC(=O)NC1=O]

A mixture of 3-amino-4-methoxybenzoic acid (1.67 g, 10.0 mmol), acrylic acid (1.10 g, 15.0 mmol) in toluene (16 mL) was stirred in a round bottom flask at 100° C. overnight. Then the mixture was added acetic acid (12 mL) and urea (1.12 g, 20 mmol) and stirred at 120° C. overnight. The reaction was concentrated to dryness and added acetic acid (20 mL). The mixture reaction was stirred at 120° C. overnight, then evaporated in vacuum to afford the crude product, which was further purified with silica gel column chromatography (DCM:MeOH=100:0˜90:10 gradient elution) to give the product (1.28 g, 48%). [M+H]⁺=265.1.

### Step 2: 3-(tert-butyl)-N-(4-(5-(4-(1-(3-(2,4-dioxotetrahydropyrimidin-1(2H)-yl)-4-methoxybenzoyl)piperidin-4-yl)phenyl)-1H-pyrazolo[3,4-b]pyridin-3-yl)-2-fluorobenzyl)-1,2,4-oxadiazole-5-carboxamide

A mixture of 3-(2,4-dioxotetrahydropyrimidin-1(2H)-yl)-4-methoxybenzoic acid (26 mg, 0.1 mmol) and HATU (38 mg, 0.1 mmol) in DMF (1 mL) was stirred in a round bottom flask at room temperature for 1 hour. Then the mixture was added 3-(tert-butyl)-N-(2-fluoro-4-(5-(4-(piperidin-4-yl)phenyl)-1H-pyrazolo[3,4-b]pyridin-3-yl)benzyl)-1,2,4-oxadiazole-5-carboxamide (62 mg, 0.1 mmol) and DIPEA (52 mg, 0.4 mmol) and stirred at room temperature overnight. The reaction was further purified with C18 column chromatography (0.1% FA in water:acetonitrile=60:40˜20:80 gradient elution) to give the product (40 mg, 50%). ¹H NMR (400 MHz, DMSO) δ_{H} 10.37 (s, 1H), 9.96 (s, 1H), 8.88 (s, 1H), 8.72 (s, 1H), 8.01-7.93 (m, 1H), 7.89 (d, J=12.8 Hz, 1H), 7.84-7.74 (m, 2H), 7.62-7.51 (m, 1H), 7.51-7.39 (m, 4H), 7.18 (d, J=7.6 Hz, 1H), 4.59 (s, 2H), 3.86 (s, 3H), 3.62 (s, 2H), 2.99-2.83 (m, 2H), 2.76-2.63 (m, 2H), 2.02-1.79 (m, 2H), 1.78-1.62 (m, 2H), 1.37 (s, 9H); [M+H]⁺=800.4.

### Example 19: 3-(tert-butyl)-N-(4-(5-(4-(1-(4-(2,6-dioxopiperidin-3-yl)phenethyl)piperidin-4-yl)phenyl)-1H-pyrazolo[3,4-b]pyridin-3-yl)-2-fluoro-3-methylbenzyl)-1,2,4-oxadiazole-5-carboxamide

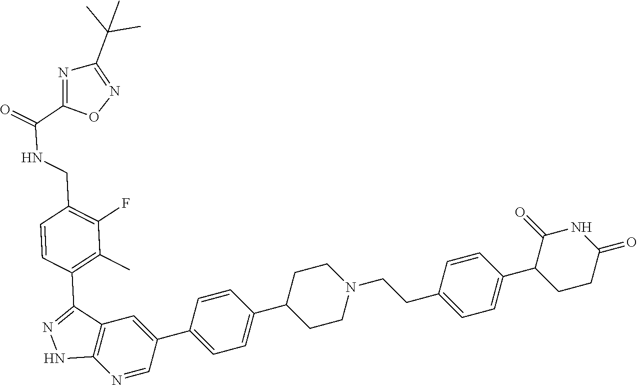 [SMILES: Cc1c(-c2n[nH]c3ncc(-c4ccc(C5CCN(CCc6ccc(C7CCC(=O)NC7=O)cc6)CC5)cc4)cc23)ccc(CNC(=O)c2nc(C(C)(C)C)no2)c1F]

The titled compound was synthesized in the procedures similar to Example 1. ¹H NMR (400 MHz, DMSO) δ_{H} 13.99 (s, 1H), 10.85 (s, 1H), 9.94 (t, J=6.0 Hz, 1H), 8.89 (s, 1H), 8.29 (s, 1H), 7.73 (d, J=6.0 Hz, 2H), 7.49 (d, J=8.0 Hz, 1H), 7.42-7.31 (m, 3H), 7.32-7.10 (m, 4H), 4.59 (d, J=6.0 Hz, 2H), 3.83 (d, J=6.0 Hz, 1H), 3.14-2.77 (m, 4H), 2.73-2.59 (m, 3H), 2.53 (s, 1H), 2.48-2.43 (m, 1H), 2.36 (s, 4H), 2.19 (dd, J=20.0, 12.0 Hz, 2H), 2.11-1.98 (m, 2H), 1.96-1.62 (m, 5H), 1.37 (s, 9H); [M+H]⁺=783.4.

### Example 20: 3-(tert-butyl)-N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylbenzyl)-1,2,4-oxadiazole-5-carboxamide

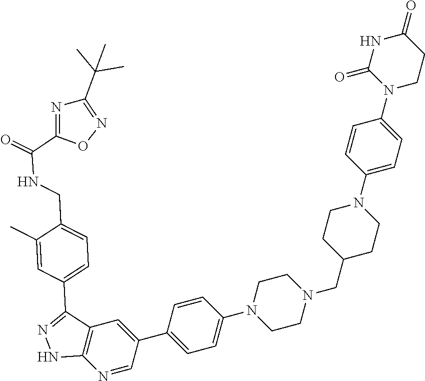 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1nc(C(C)(C)C)no1]

The titled compound was synthesized in the procedures similar to Example 11. ¹H NMR (400 MHz, DMSO) δ_{H} 13.99 (s, 1H), 10.85 (s, 1H), 9.94 (t, J=6.0 Hz, 1H), 8.89 (s, 1H), 8.29 (s, 1H), 7.73 (d, J=6.0 Hz, 2H), 7.49 (d, J=8.0 Hz, 1H), 7.42-7.31 (m, 3H), 7.32-7.10 (m, 4H), 4.59 (d, J=6.0 Hz, 2H), 3.83 (d, J=6.0 Hz, 1H), 3.14-2.77 (m, 4H), 2.73-2.59 (m, 3H), 2.53 (s, 1H), 2.48-2.43 (m, 1H), 2.36 (s, 4H), 2.19 (dd, J=20.0, 12.0 Hz, 2H), 2.11-1.98 (m, 2H), 1.96-1.62 (m, 5H), 1.37 (s, 9H); [M+H]⁺=836.5.

### Example 21: (R)-3-(tert-butyl)-N-(1-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylphenyl)ethyl)-1,2,4-oxadiazole-5-carboxamide

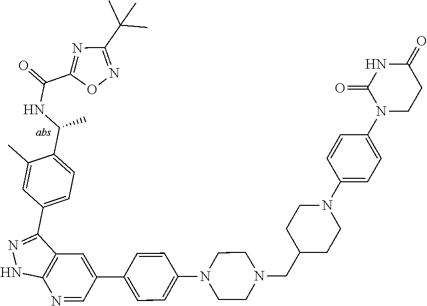 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.78 (s, 1H), 10.27 (s, 1H), 9.92 (d, J=8.0 Hz, 1H), 8.82 (s, 1H), 8.58 (s, 1H), 7.97-7.77 (m, 2H), 7.69-7.55 (m, 3H), 7.18-7.0 (m, 4H), 6.93 (d, J=8.0 Hz, 2H), 5.34 (d, J=8.0 Hz, 1H), 3.78-3.61 (m, 4H), 3.21 (s, 4H), 2.75-2.60 (m, 5H), 2.52 (s, 3H), 2.24 (d, J=8.0 Hz, 2H), 1.82 (d, J=12.0 Hz, 2H), 1.73 (s, 1H), 1.53 (d, J=8.0 Hz, 3H), 1.36 (s, 9H), 1.29-1.19 (m, 3H); [M+H]⁺=850.5.

### Example 22: 5-(tert-butyl)-N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylbenzyl)-1,2,4-oxadiazole-3-carboxamide

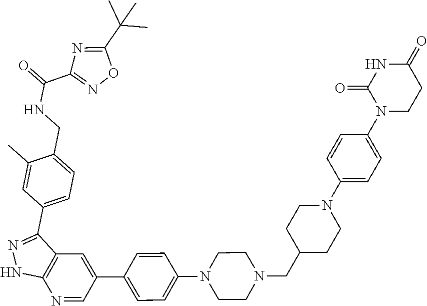 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1noc(C(C)(C)C)n1]

The titled compound was synthesized in the procedures similar to Example 1. ¹H NMR (400 MHz, DMSO) δ_{H} 13.78 (s, 1H), 10.27 (s, 1H), 9.46 (s, 1H), 8.81 (s, 1H), 8.58 (s, 1H), 7.88 (s, 2H), 7.69 (s, 2H), 7.40 (s, 1H), 7.20-7.03 (m, 4H), 6.93 (s, 1H), 4.51 (s, 2H), 3.69 (s, 4H), 3.33-3.18 (m, 4H), 2.74-2.64 (m, 5H), 2.44 (s, 3H), 2.24 (s, 2H), 1.83 (s, 2H), 1.57-1.37 (m, 9H), 1.23 (s, 3H); [M+H]⁺=836.5.

### Example 23: 3-(tert-butyl)-N-(4-(5-(4-(1-(2-(1-(3-chloro-4-(2,4-dioxotetrahydropyrimidin-1(2H)-yl)phenyl)piperidin-4-yl)ethyl)piperidin-4-yl)phenyl)-1H-pyrazolo[3,4-b]pyridin-3-yl)-2-methylbenzyl)-1,2,4-oxadiazole-5-carboxamide

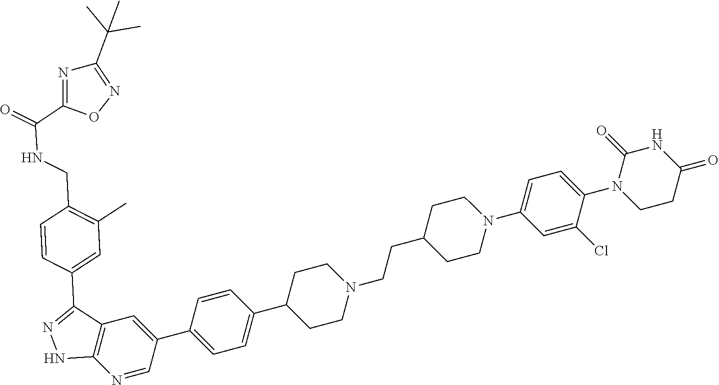 [SMILES: Cc1cc(-c2n[nH]c3ncc(-c4ccc(C5CCN(CCC6CCN(c7ccc(N8CCC(=O)NC8=O)c(Cl)c7)CC6)CC5)cc4)cc23)ccc1CNC(=O)c1nc(C(C)(C)C)no1]

The titled compound was synthesized in the procedures similar to Example 1. ¹H NMR (400 MHz, DMSO) δ_{H} 13.82 (s, 1H), 10.35 (s, 1H), 9.84 (s, 1H), 8.82 (s, 1H), 8.63 (s, 1H), 7.89 (s, 2H), 7.72 (d, J=8.0 Hz, 2H), 7.41-7.29 (m, 3H), 7.21 (d, J=8.0 Hz, 1H), 6.99 (s, 1H), 6.91 (d, J=8.0 Hz, 1H), 4.50 (d, J=4.0 Hz, 2H), 3.72 (d, J=12.0 Hz, 2H), 3.64-3.44 (m, 2H), 3.02 (s, 2H), 2.79-2.63 (m, 5H), 2.61-2.51 (m, 2H), 2.43 (s, 3H), 1.88 (s, 2H), 1.82-1.62 (m, 6H), 1.44 (s, 3H), 1.34 (s, 9H), 1.26-1.15 (s, 2H); [M+H]⁺=883.6.

### Example 24: 3-(tert-butyl)-N-(4-(5-(4-(1-(3-(4-((2,6-dioxopiperidin-3-yl)amino)-3-methylphenoxy)propyl)piperidin-4-yl)phenyl)-1H-pyrazolo[3,4-b]pyridin-3-yl)-2-methylbenzyl)-1,2,4-oxadiazole-5-carboxamide

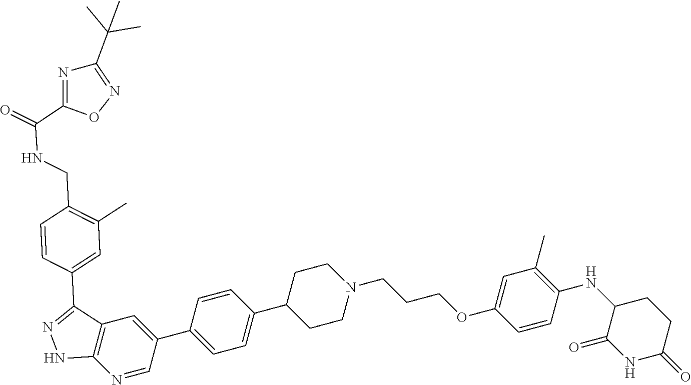 [SMILES: Cc1cc(-c2n[nH]c3ncc(-c4ccc(C5CCN(CCCOc6ccc(NC7CCC(=O)NC7=O)c(C)c6)CC5)cc4)cc23)ccc1CNC(=O)c1nc(C(C)(C)C)no1]

The titled compound was synthesized in the procedures similar to Example 1. ¹H NMR (400 MHz, DMSO) δ_{H} 13.82 (s, 1H), 10.82 (s, 1H), 9.83 (s, 1H), 8.82 (s, 1H), 8.63 (s, 1H), 7.87 (s, 2H), 7.74 (s, 2H), 7.44-7.28 (m, 3H), 6.79-6.37 (m, 3H), 4.64 (s, 1H), 4.50 (s, 2H), 4.24 (s, 1H), 3.90 (s, 2H), 2.82-2.66 (m, 4H), 2.57-2.54 (m, 2H), 2.42 (s, 3H), 2.08 (s, 5H), 1.95-1.63 (m, 8H), 1.34 (s, 9H), 1.20 (s, 2H); [M+H]⁺=824.5.

### Example 25: 3-(tert-butyl)-N-(4-(5-(4-(1-(2-(4-(2,6-dioxopiperidin-3-yl)naphthalen-1-yl)ethyl)piperidin-4-yl)phenyl)-1H-pyrazolo[3,4-b]pyridin-3-yl)-2-methylbenzyl)-1,2,4-oxadiazole-5-carboxamide

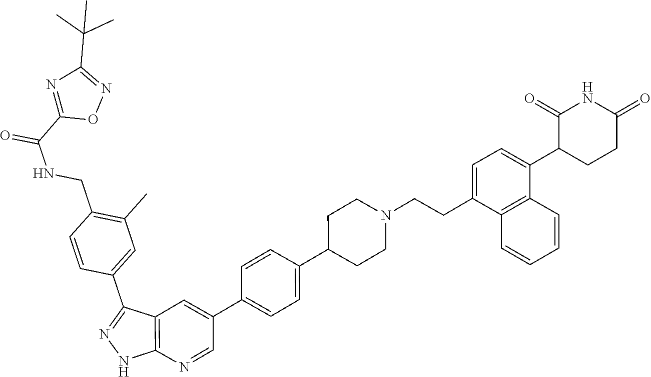 [SMILES: Cc1cc(-c2n[nH]c3ncc(-c4ccc(C5CCN(CCc6ccc(C7CCC(=O)NC7=O)c7ccccc67)CC5)cc4)cc23)ccc1CNC(=O)c1nc(C(C)(C)C)no1]

The titled compound was synthesized in the procedures similar to Example 1. ¹H NMR (400 MHz, DMSO) δ_{H} 13.83 (s, 1H), 10.91 (s, 1H), 9.84 (s, 2H), 8.83 (s, 2H), 8.64 (s, 1H), 8.24 (s, 1H), 8.18-8.09 (m, 1H), 8.08-7.98 (m, 1H), 7.88 (s, 2H), 7.98-7.86 (m, 2H), 7.82-7.69 (m, 3H), 7.48-7.36 (m, 4H), 7.34-7.25 (m, 1H), 4.65 (s, 2H), 4.51 (s, 2H), 3.26-3.14 (m, 4H), 2.43 (s, 3H), 2.25-2.03 (m, 5H), 1.88-1.66 (m, 5H), 1.36 (s, 9H); [M+H]⁺=815.5.

### Example 26: 3-(tert-butyl)-N-(4-(5-(4-(1-(2-(4-(2,6-dioxopiperidin-3-yl)-5,6,7,8-tetrahydronaphthalen-1-yl)ethyl)piperidin-4-yl)phenyl)-1H-pyrazolo[3,4-b]pyridin-3-yl)-2-methylbenzyl)-1,2,4-oxadiazole-5-carboxamide

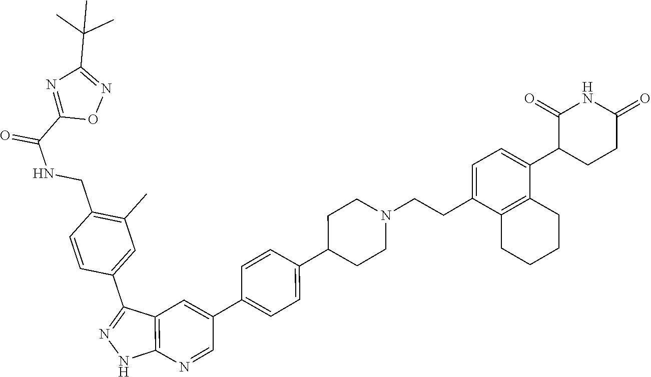 [SMILES: Cc1cc(-c2n[nH]c3ncc(-c4ccc(C5CCN(CCc6ccc(C7CCC(=O)NC7=O)c7c6CCCC7)CC5)cc4)cc23)ccc1CNC(=O)c1nc(C(C)(C)C)no1]

The titled compound was synthesized in the procedures similar to Example 1. ¹H NMR (400 MHz, DMSO) δ_{H} 13.83 (s, 1H), 10.78 (s, 1H), 9.84 (s, 1H), 8.82 (s, 1H), 8.64 (s, 1H), 7.88 (s, 2H), 7.74 (d, J=4.0 Hz, 2H), 7.39 (s, 3H), 7.13 (s, 1H), 7.00-6.82 (m, 1H), 4.50 (s, 1H), 3.18 (s, 1H), 2.85-2.50 (m, 15H), 2.43 (s, 3H), 2.12 (s, 1H), 1.98-1.58 (m, 10H), 1.34 (s, 9H); [M+H]⁺=819.5.

### Example 27: 3-(tert-butyl)-N-(4-(5-(4-(1-(3-(4-(2,6-dioxopiperidin-3-yl)-3-methylphenoxy)propyl)piperidin-4-yl)phenyl)-1H-pyrazolo[3,4-b]pyridin-3-yl)-2-methylbenzyl)-1,2,4-oxadiazole-5-carboxamide

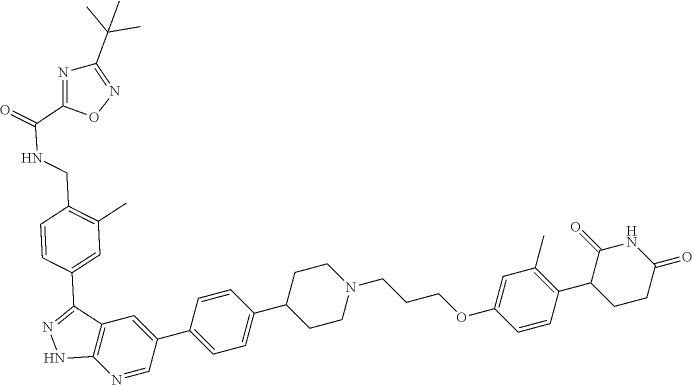 [SMILES: Cc1cc(-c2n[nH]c3ncc(-c4ccc(C5CCN(CCCOc6ccc(C7CCC(=O)NC7=O)c(C)c6)CC5)cc4)cc23)ccc1CNC(=O)c1nc(C(C)(C)C)no1]

The titled compound was synthesized in the procedures similar to Example 1. ¹H NMR (400 MHz, DMSO) δ_{H} 13.84 (s, 1H), 10.78 (s, 1H), 9.84 (s, 1H), 9.11 (s, 1H), 8.83 (s, 1H), 8.64 (s, 1H), 7.87 (s, 2H), 7.79 (d, J=8.0 Hz, 2H), 7.43-7.30 (m, 3H), 7.01 (d, J=8.0 Hz, 1H), 6.86-6.67 (m, 2H), 4.50 (d, J=4.0 Hz, 2H), 4.05 (s, 2H), 3.99-3.88 (m, 1H), 3.73-3.61 (m, 2H), 3.28-3.22 (m, 2H), 3.18-3.04 (m, 2H), 2.91 (s, 1H), 2.78-2.60 (m, 1H), 2.43 (s, 3H), 2.22 (s, 3H), 2.18-2.02 (m, 6H), 1.96-1.84 (m, 3H), 1.34 (s, 9H); [M+H]⁺=809.5.

### Example 28: 3-(tert-butyl)-N-(4-(5-(4-(1-(2-(1-(4-(2,4-dioxotetrahydropyrimidin-1(2H)-yl)-3-methoxyphenyl)piperidin-4-yl)ethyl)piperidin-4-yl)phenyl)-1H-pyrazolo[3,4-b]pyridin-3-yl)-2-methylbenzyl)-1,2,4-oxadiazole-5-carboxamide

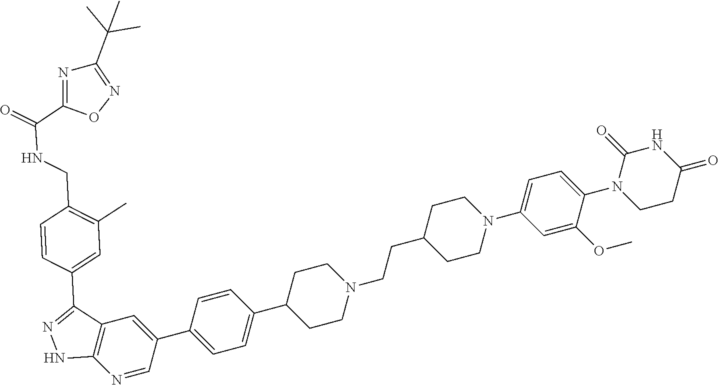 [SMILES: COc1cc(N2CCC(CCN3CCC(c4ccc(-c5cnc6[nH]nc(-c7ccc(CNC(=O)c8nc(C(C)(C)C)no8)c(C)c7)c6c5)cc4)CC3)CC2)ccc1N1CCC(=O)NC1=O]

The titled compound was synthesized in the procedures similar to Example 1. ¹H NMR (400 MHz, DMSO) δ_{H} 13.82 (s, 1H), 10.17 (s, 1H), 9.83 (s, 1H), 8.82 (s, 1H), 8.63 (s, 1H), 7.87 (s, 2H), 7.72 (d, J=8.0 Hz, 2H), 7.42-7.23 (m, 3H), 6.98 (d, J=8.0 Hz, 1H), 6.56 (s, 1H), 6.46 (d, J=8.0 Hz, 1H), 4.50 (s, 2H), 3.74 (s, 3H), 3.71 (d, J=12.0 Hz, 1H), 3.47 (s, 2H), 3.05 (s, 2H), 2.74-2.55 (m, 6H), 2.42 (s, 3H), 2.07 (s, 2H), 1.85-1.62 (m, 6H), 1.45 (s, 3H), 1.34 (s, 9H), 1.30-1.18 (m, 3H); [M+H]⁺=879.8.

### Example 29: 3-(tert-butyl)-N-(4-(5-(4-(1-(4-((2,6-dioxopiperidin-3-yl)oxy)phenethyl)piperidin-4-yl)phenyl)-1H-pyrazolo[3,4-b]pyridin-3-yl)-2-methylbenzyl)-1,2,4-oxadiazole-5-carboxamide

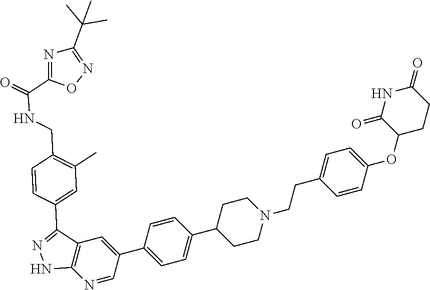 [SMILES: Cc1cc(-c2n[nH]c3ncc(-c4ccc(C5CCN(CCc6ccc(OC7CCC(=O)NC7=O)cc6)CC5)cc4)cc23)ccc1CNC(=O)c1nc(C(C)(C)C)no1]

The titled compound was synthesized in the procedures similar to Example 1. ¹H NMR (400 MHz, DMSO) δ_{H} 13.82 (s, 1H), 10.90 (s, 1H), 9.84 (s, 1H), 8.82 (s, 1H), 8.63 (s, 1H), 7.88 (s, 2H), 7.72 (d, J=8.0 Hz, 2H), 7.38 (t, J=8.0 Hz, 4H), 7.13 (d, J=7.6 Hz, 2H), 6.92 (d, J=8.0 Hz, 1H), 5.14 (s, 1H), 4.50 (s, 1H), 3.06 (d, J=8.0 Hz, 2H), 2.80-2.61 (m, 7H), 2.43 (s, 3H), 2.22-2.02 (m, 4H), 1.82-1.63 (m, 3H), 1.34 (s, 9H); [M+H]⁺=781.5.

### Example 30: 3-(tert-butyl)-N-(4-(5-(4-(1-(3-(4-(2,6-dioxopiperidin-3-yl)-3-methoxyphenoxy)propyl)piperidin-4-yl)phenyl)-1H-pyrazolo[3,4-b]pyridin-3-yl)-2-methylbenzyl)-1,2,4-oxadiazole-5-carboxamide

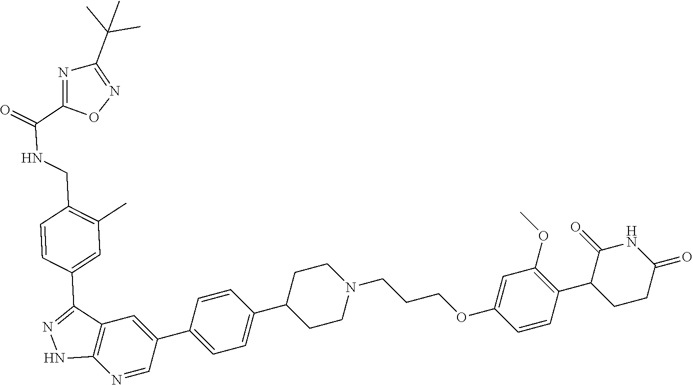 [SMILES: COc1cc(OCCCN2CCC(c3ccc(-c4cnc5[nH]nc(-c6ccc(CNC(=O)c7nc(C(C)(C)C)no7)c(C)c6)c5c4)cc3)CC2)ccc1C1CCC(=O)NC1=O]

The titled compound was synthesized in the procedures similar to Example 1. ¹H NMR (400 MHz, DMSO) δ_{H} 13.81 (s, 1H), 10.68 (s, 1H), 9.82 (s, 1H), 8.81 (s, 1H), 8.62 (s, 1H), 7.86 (s, 2H), 7.74 (s, 2H), 7.37 (s, 3H), 7.00 (s, 1H), 6.53 (s, 1H), 4.49 (s, 2H), 4.02 (s, 3H), 3.90-3.77 (m, 2H), 3.69 (s, 3H), 2.70-2.60 (m, 8H), 2.41-2.36 (m, 3H), 2.05-1.65 (m, 8H), 1.33 (s, 9H); [M+H]⁺=825.5.

### Example 31: 1-(tert-butyl)-N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylbenzyl)-1H-1,2,3-triazole-4-carboxamide

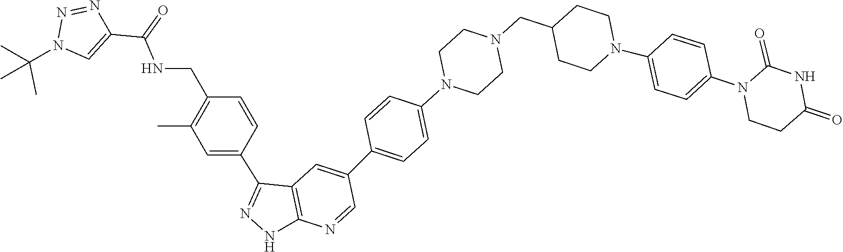 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1cn(C(C)(C)C)nn1]

### Step 1: N-(4-(5-bromo-1-(tetrahydro-2H-pyran-2-yl)-1H-pyrazolo[3,4-b]pyridin-3-yl)-2-methylbenzyl)-1-(tert-butyl)-1H-1,2,3-triazole-4-carboxamide

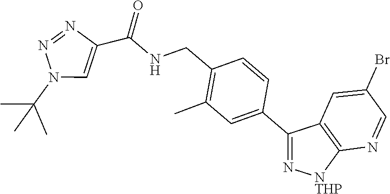

To a solution of 5-bromo-3-iodo-1-(tetrahydro-2H-pyran-2-yl)-1H-pyrazolo[3,4-b]pyridine (0.2 g, 0.5 mmol) in dioxane (35 mL) and H₂O (6 mL) was added 1-(tert-butyl)-N-(2-methyl-4-(4,4,5,5-tetramethyl-1,3,2-dioxaborolan-2-yl)benzyl)-1H-1,2,3-triazole-4-carboxamide (0.2 g, 0.5 mmol), Pd(dppf)Cl₂ (41 mg, 0.05 mmol), K₂CO₃ (0.21 g, 1.5 mmoL). The mixture was stirred at 100° C. for 18 hours, concentrated and dissolved in H₂O (30 mL), extracted with EtOAc (30 mL*2). The organic layer was concentrated and purified by pre-TLC with PE/EtOAc (1:2) to give the product (0.3 g, crude).

### Step 2: tert-butyl 4-(4-(3-(4-((1-(tert-butyl)-1H-1,2,3-triazole-4-carboxamido)methyl)-3-methylphenyl)-1-(tetrahydro-2H-pyran-2-yl)-1H-pyrazolo[3,4-b]pyridin-5-yl)phenyl)piperazine-1-carboxylate

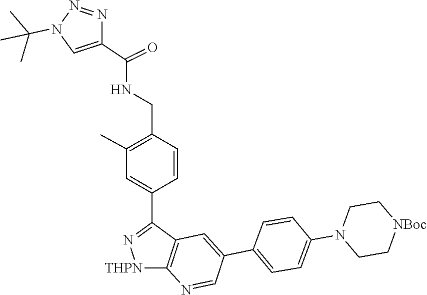

To a solution of N-(4-(5-bromo-1-(tetrahydro-2H-pyran-2-yl)-1H-pyrazolo[3,4-b]pyridin-3-yl)-2-methylbenzyl)-1-(tert-butyl)-1H-1,2,3-triazole-4-carboxamide (0.3 g, 0.54 mmol) in dioxane/H₂O (5:1, 40 mL) was added tert-butyl 4-(4-(4,4,5,5-tetramethyl-1,3,2-dioxaborolan-2-yl)phenyl)piperazine-1-carboxylate (0.21 g, 0.54 mmol), K₂CO₃ (0.27 g, 1.6 mmol) and Pd(dppf)Cl₂·CH₂Cl₂ (44 mg, 0.054 mmol). The mixture was stirred at 100° C. for 18 hours. Vaporated 1,4-dioxane in vacuo, then mixture was extracted with water and EtOAc (30 mL*2). The organic phase was combined and purified by pre-TLC with PE/EtOAc (1:2) to give the product (250 mg, 63%).

### Step 3: 1-(tert-butyl)-N-(2-methyl-4-(5-(4-(piperazin-1-yl)phenyl)-1H-pyrazolo[3,4-b]pyridin-3-yl)benzyl)-1H-1,2,3-triazole-4-carboxamide hydrochloride

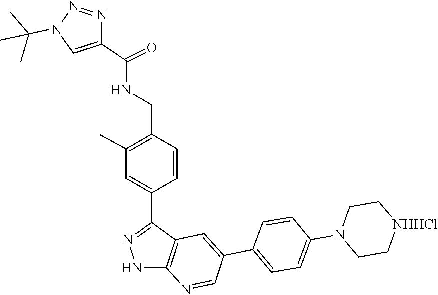

To a solution of tert-butyl 4-(4-(3-(4-((1-(tert-butyl)-1H-1,2,3-triazole-4-carboxamido)methyl)-3-methylphenyl)-1-(tetrahydro-2H-pyran-2-yl)-1H-pyrazolo[3,4-b]pyridin-5-yl)phenyl)piperazine-1-carboxylate (0.25 g, 0.34 mmol) in dioxane (3 mL) was added HCl/dioxane (4 N, 30 mL). The mixture was stirred at 20-30° C. for 2 hours, and filtered to get the filter cake. washed the filter cake with DCM and dried it to give the product, which was used in next step directly. ¹H NMR (400 MHz, DMSO) δ_{H} 13.80 (br, 1H), 9.23 (s, 2H), 8.99 (s, 1H), 8.81 (s, 1H), 8.71 (s, 1H), 8.57 (s, 1H), 7.90-7.81 (m, 2H), 7.72 (d, J=8.4 Hz, 2H), 7.36 (d, J=7.6 Hz, 1H), 7.10 (d, J=8.0 Hz, 2H), 4.49 (d, J=5.2 Hz, 2H), 3.42 (s, 4H), 3.22 (s, 4H), 2.42 (s, 3H), 1.62 (s, 9H). [M+H]⁺=550.7.

### Step 4: 1-(tert-butyl)-N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylbenzyl)-1H-1,2,3-triazole-4-carboxamide

To a solution of 1-(tert-butyl)-N-(2-methyl-4-(5-(4-(piperazin-1-yl)phenyl)-1H-pyrazolo[3,4-b]pyridin-3-yl)benzyl)-1H-1,2,3-triazole-4-carboxamide hydrochloride (0.15 g, 0.26 mmol) in DCM/EtOH (5:1, 30 mL) was added 1-(4-(2,4-dioxotetrahydropyrimidin-1(2H)-yl)phenyl)piperidine-4-carbaldehyde (0.07 g, 0.26 mmol) and NaOAc (0.06 mg, 0.8 mmol). The mixture was stirred at 20-30° C. for 1 hour, then NaBH(OAc)₃ (0.16 g, 0.8 mmol) was added and mixture was stirred at 20-30° C. for 1 hour, concentrated, H₂O (30 mL) was added and the mixture was extracted with DCM/MeOH (5:1, 30 mL*2). The organic phase was combined, concentrated and purified by pre-TLC with DCM/MeOH (10:1) to give the product (24.52 mg, 11%). ¹H NMR (400 MHz, DMSO) δ 13.73 (s, 1H), 10.24 (s, 1H), 8.98 (br, 1H), 8.79 (s, 1H), 8.70 (s, 1H), 8.55 (br, 1H), 7.84 (br, 2H), 7.66 (d, J=8.4 Hz, 2H), 7.35 (d, J=7.6 Hz, 1H), 7.11 (d, J=8.8 Hz, 2H), 7.03 (d, J=7.6 Hz, 2H), 6.91 (d, J=8.4 Hz, 2H), 4.49 (s, 2H), 3.72-3.62 (m, 4H), 3.19 (s, 4H), 2.71-2.50 (m, 7H), 2.42 (s, 3H), 2.21 (br, 2H), 1.85-1.65 (m, 3H), 1.62 (s, 9H), 1.22 (br, 3H). [M+H]⁺=835.9.

### Example 32: 1-(tert-butyl)-N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylbenzyl)-1H-pyrazole-4-carboxamide

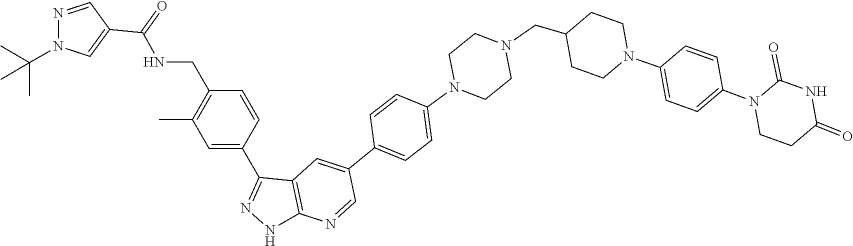 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1cnn(C(C)(C)C)c1]

The titled compound was synthesized in the procedures similar to Example 31. ¹H NMR (400 MHz, DMSO) δ 13.77 (s, 1H), 10.27 (s, 1H), 8.82 (s, 1H), 8.57 (s, 1H), 8.48 (s, 1H), 8.35 (s, 1H), 7.99-7.85 (m, 3H), 7.68 (d, J=7.2 Hz, 2H), 7.39 (d, J=7.6 Hz, 1H), 7.13 (d, J=7.6 Hz, 2H), 7.07 (d, J=7.2 Hz, 2H), 6.93 (d, J=8.4 Hz, 2H), 4.47 (s, 2H), 3.70 (br, 4H), 3.21 (s, 4H), 2.75-2.50 (m, 7H), 2.43 (s, 3H), 2.24 (br, 2H), 1.86-1.68 (m, 3H), 1.53 (s, 9H), 1.24 (s, 3H). [M+H]⁺=834.6.

### Example 33: 3-(tert-butyl)-N-((1R)-1-(4-(5-(4-(4-(4-(2,6-dioxopiperidin-3-yl)phenethyl)piperazin-1-yl)phenyl)-1H-pyrazolo[3,4-b]pyridin-3-yl)-2-methylphenyl)ethyl)-1,2,4-oxadiazole-5-carboxamide

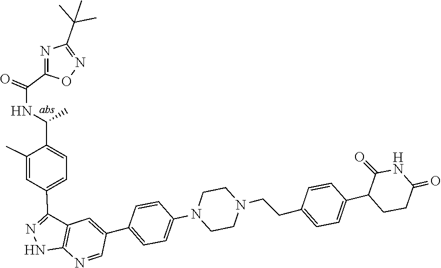 [SMILES: Cc1cc(-c2n[nH]c3ncc(-c4ccc(N5CCN(CCc6ccc(C7CCC(=O)NC7=O)cc6)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.79 (s, 1H), 10.84 (s, 1H), 9.91 (d, J=7.2 Hz, 1H), 8.82 (s, 1H), 8.59 (s, 1H), 7.93 (d, J=7.2 Hz, 1H), 7.86 (s, 1H), 7.70 (s, 2H), 7.62 (d, J=7.8 Hz, 1H), 7.29-7.03 (m, 8H), 5.35 (s, 1H), 3.83 (s, 1H), 3.22 (s, 5H), 2.84-2.56 (m, 8H), 2.18 (d, J=11.7 Hz, 1H), 2.04 (s, 1H), 1.54 (d, J=6.1 Hz, 3H), 1.37 (s, 11H); [M+H]⁺=780.8.

### Example 34: 3-(tert-butyl)-N-((1R)-1-(4-(5-(4-(4-(4-(2,6-dioxopiperidin-3-yl)-3-fluorophenethyl)piperazin-1-yl)phenyl)-1H-pyrazolo[3,4-b]pyridin-3-yl)-2-methylphenyl)ethyl)-1,2,4-oxadiazole-5-carboxamide

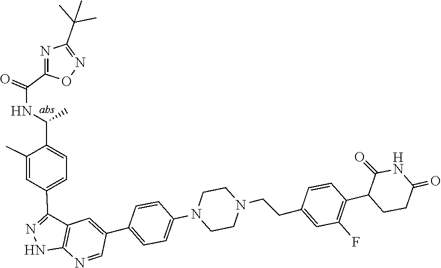 [SMILES: Cc1cc(-c2n[nH]c3ncc(-c4ccc(N5CCN(CCc6ccc(C7CCC(=O)NC7=O)c(F)c6)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.78 (s, 1H), 10.88 (s, 2H), 9.91 (d, J=6.6 Hz, 1H), 8.82 (s, 1H), 8.58 (s, 1H), 7.93 (d, J=7.7 Hz, 1H), 7.86 (s, 1H), 7.69 (d, J=7.4 Hz, 2H), 7.62 (d, J=7.9 Hz, 1H), 7.30-7.00 (m, 6H), 5.36 (s, 1H), 4.01 (d, J=7.2 Hz, 1H), 3.22 (s, 4H), 2.85-2.54 (m, 12H), 2.19 (d, J=12.3 Hz, 2H), 2.00 (s, 2H), 1.54 (d, J=5.7 Hz, 3H), 1.37 (s, 9H); [M+H]⁺=798.8.

### Example 35: 5-(tert-butyl)-N-((1R)-1-(4-(5-(4-(4-(4-(2,6-dioxopiperidin-3-yl)phenethyl)piperazin-1-yl)phenyl)-1H-pyrazolo[3,4-b]pyridin-3-yl)-2-methylphenyl)ethyl)-1,2,4-oxadiazole-3-carboxamide

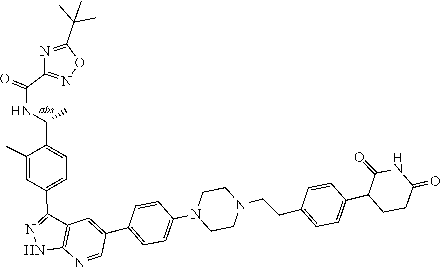 [SMILES: Cc1cc(-c2n[nH]c3ncc(-c4ccc(N5CCN(CCc6ccc(C7CCC(=O)NC7=O)cc6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.77 (s, 1H), 10.83 (s, 1H), 9.50 (d, J=6.4 Hz, 1H), 8.82 (s, 1H), 8.58 (s, 1H), 7.90 (d, J=7.2 Hz, 1H), 7.85 (s, 1H), 7.68 (d, J=8.4 Hz, 2H), 7.60 (d, J=8.0 Hz, 1H), 7.25-7.05 (m, 6H), 5.36 (s, 1H), 3.87-3.77 (m, 1H), 3.23 (s, 4H), 2.96 (s, 2H), 2.83-2.55 (m, 11H), 2.25-2.12 (m, 1H), 2.04 (s, 1H), 1.51 (d, J=6.1 Hz, 3H), 1.42 (s, 9H); [M+H]⁺=780.8.

### Example 36: 5-(tert-butyl)-N-((1R)-1-(4-(5-(4-(4-(4-(2,6-dioxopiperidin-3-yl)-3-fluorophenethyl)piperazin-1-yl)phenyl)-1H-pyrazolo[3,4-b]pyridin-3-yl)-2-methylphenyl)ethyl)-1,2,4-oxadiazole-3-carboxamide

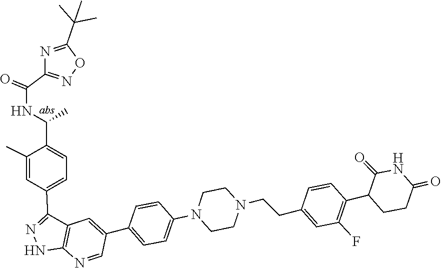 [SMILES: Cc1cc(-c2n[nH]c3ncc(-c4ccc(N5CCN(CCc6ccc(C7CCC(=O)NC7=O)c(F)c6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.77 (s, 1H), 10.88 (s, 1H), 9.49 (d, J=7.1 Hz, 1H), 8.82 (s, 1H), 8.58 (s, 1H), 7.91 (d, J=7.1 Hz, 1H), 7.85 (s, 1H), 7.69 (d, J=7.0 Hz, 2H), 7.60 (d, J=8.0 Hz, 1H), 7.25-7.00 (m, 5H), 5.42-5.31 (m, 1H), 4.01 (d, J=9.6 Hz, 1H), 3.23 (s, 3H), 2.96 (s, 2H), 2.87-2.54 (m, 12H), 2.21-2.17 (m, 1H), 2.00 (s, 1H), 1.55-1.48 (m, 3H), 1.42 (s, 9H); [M+H]⁺=798.8.

### Example 37: (R)-5-(tert-butyl)-N-(1-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

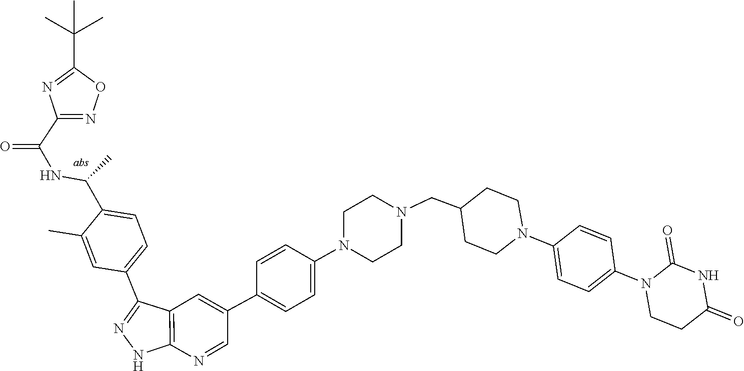 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.77 (s, 1H), 10.27 (s, 1H), 9.49 (d, J=7.1 Hz, 1H), 8.82 (s, 1H), 8.58 (s, 1H), 7.91 (d, J=7.1 Hz, 1H), 7.85 (s, 1H), 7.69 (d, J=7.4 Hz, 2H), 7.60 (d, J=8.0 Hz, 1H), 7.18-7.03 (m, 4H), 6.93 (d, J=6.1 Hz, 3H), 5.36 (s, 1H), 3.69 (s, 5H), 3.22 (s, 4H), 2.74-2.54 (m, 9H), 2.25 (s, 2H), 1.87-1.68 (m, 4H), 1.51 (d, J=5.5 Hz, 3H), 1.42 (s, 9H), 1.24 (s, 4H); [M+H]⁺=850.9.

### Example 38: (R)-5-(tert-butyl)-N-(1-(4-(5-(4-(4-((1-(4-(2,4-dioxotetrahydropyrimidin-1(2H)-yl)-3-fluorophenyl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

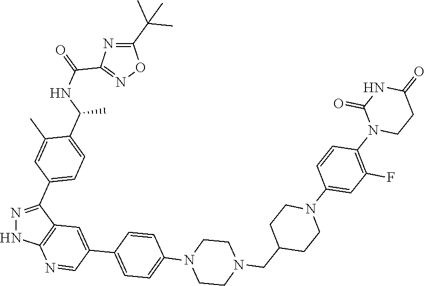 [SMILES: Cc1cc(-c2n[nH]c3ncc(-c4ccc(N5CCN(CC6CCN(c7ccc(N8CCC(=O)NC8=O)c(F)c7)CC6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.77 (s, 1H), 10.38 (s, 1H), 9.49 (d, J=7.0 Hz, 1H), 8.82 (s, 1H), 8.58 (s, 1H), 7.91 (d, J=8.3 Hz, 1H), 7.85 (s, 1H), 7.69 (d, J=7.1 Hz, 2H), 7.60 (d, J=7.8 Hz, 1H), 7.18 (t, J=9.1 Hz, 1H), 7.07 (d, J=7.6 Hz, 2H), 6.85-6.73 (m, 2H), 5.42-5.32 (m, 1H), 3.76 (d, J=12.3 Hz, 2H), 3.62 (s, 2H), 3.21 (s, 4H), 2.80-2.51 (m, 11H), 2.23 (s, 2H), 1.87-1.71 (m, 3H), 1.51 (d, J=5.4 Hz, 3H), 1.42 (s, 9H), 1.26-1.20 (m, 2H); [M+H]⁺=869.0.

### Example 39: (R)-5-(tert-butyl)-N-(1-(4-(5-(4-(4-((1-(4-(2,4-dioxotetrahydropyrimidin-1(2H)-yl)-3-methylphenyl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

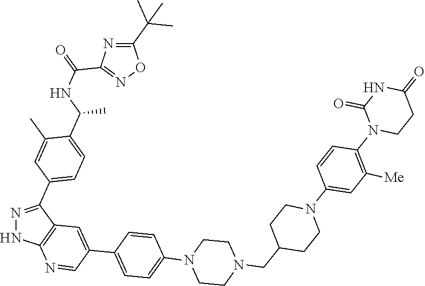 [SMILES: Cc1cc(-c2n[nH]c3ncc(-c4ccc(N5CCN(CC6CCN(c7ccc(N8CCC(=O)NC8=O)c(C)c7)CC6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.77 (s, 1H), 10.25 (s, 1H), 9.48 (d, J=5.4 Hz, 1H), 8.82 (s, 1H), 8.58 (s, 1H), 7.95-7.81 (m, 2H), 7.75-7.54 (m, 3H), 7.05 (t, J=8.2 Hz, 3H), 6.85-6.74 (m, 2H), 5.36 (s, 1H), 3.75-3.61 (m, 3H), 3.47 (s, 1H), 3.21 (s, 4H), 2.96 (s, 2H), 2.80-2.54 (m, 7H), 2.24 (s, 2H), 2.12 (s, 3H), 1.86-1.70 (m, 3H), 1.51 (d, J=5.1 Hz, 3H), 1.42 (s, 9H), 1.25-1.20 (m, 2H); [M+H]⁺=864.9.

### Example 40: 5-(tert-butyl)-N-((1R)-1-(4-(5-(4-(4-(4-(2,6-dioxopiperidin-3-yl)-3-methylphenethyl)piperazin-1-yl)phenyl)-1H-pyrazolo[3,4-b]pyridin-3-yl)-2-methylphenyl)ethyl)-1,2,4-oxadiazole-3-carboxamide

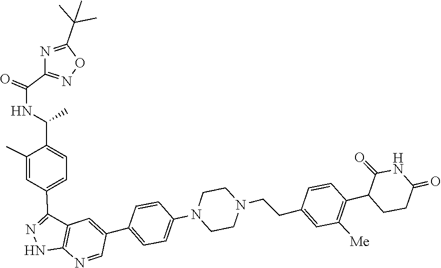 [SMILES: Cc1cc(CCN2CCN(c3ccc(-c4cnc5[nH]nc(-c6ccc([C@@H](C)NC(=O)c7noc(C(C)(C)C)n7)c(C)c6)c5c4)cc3)CC2)ccc1C1CCC(=O)NC1=O]

The titled compound was synthesized in the procedures similar to Example 1. ¹H NMR (400 MHz, DMSO) δ_{H} 13.86 (s, 1H), 10.86 (s, 1H), 9.46 (s, 1H), 8.86 (s, 1H), 8.67 (s, 1H), 7.86 (s, 2H), 7.85-7.79 (m, 2H), 7.61 (s, 1H), 7.43-7.40 (m, 2H), 7.15-7.10 (m, 3H), 5.38-5.34 (m, 2H), 4.05-4.01 (m, 2H), 3.72-3.68 (m, 2H), 3.35-3.31 (m, 2H), 3.31-2.85 (m, 4H), 2.85-2.78 (m, 1H), 2.55-2.53 (m, 4H), 2.52 (s, 3H), 2.20 (s, 3H), 2.20-2.05 (m, 1H), 2.05-1.95 (m, 1H), 1.56 (d, J=2.4 Hz, 3H), 1.44 (s, 9H); [M+H]⁺=793.8.

### Example 41: N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylbenzyl)-5-(1,1,1-trifluoro-2-methylpropan-2-yl)-1,2,4-oxadiazole-3-carboxamide

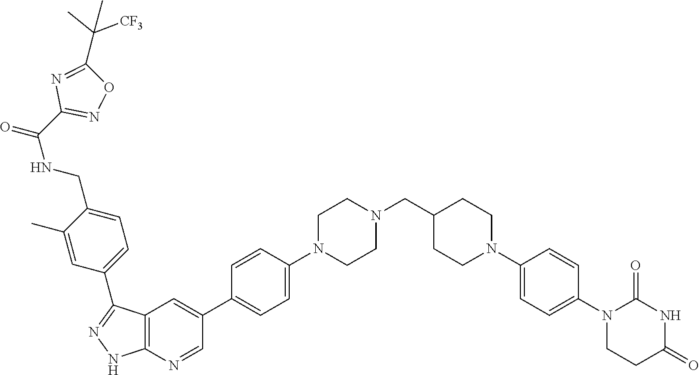 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1noc(C(C)(C)C(F)(F)F)n1]

The titled compound was synthesized in the procedures similar to Example 1. ¹H NMR (400 MHz, DMSO) δ_{H} 13.78 (s, 1H), 10.27 (s, 1H), 9.61 (s, 1H), 8.82 (s, 1H), 8.58 (s, 1H), 7.91 (d, J=10.2 Hz, 2H), 7.69 (d, J=7.8 Hz, 2H), 7.41 (d, J=7.6 Hz, 1H), 7.20-6.89 (m, 6H), 4.54 (d, J=4.2 Hz, 2H), 3.77-3.64 (m, 4H), 3.21 (s, 4H), 2.73-2.62 (m, 5H), 2.59-2.52 (m, 3H), 2.46 (s, 3H), 2.24 (s, 2H), 1.82 (d, J=11.5 Hz, 2H), 1.71 (s, 7H), 1.32-1.16 (m, 2H); [M+H]⁺=890.8.

### Example 42: N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylbenzyl)-5-(1-(trifluoromethyl)cyclopropyl)-1,2,4-oxadiazole-3-carboxamide

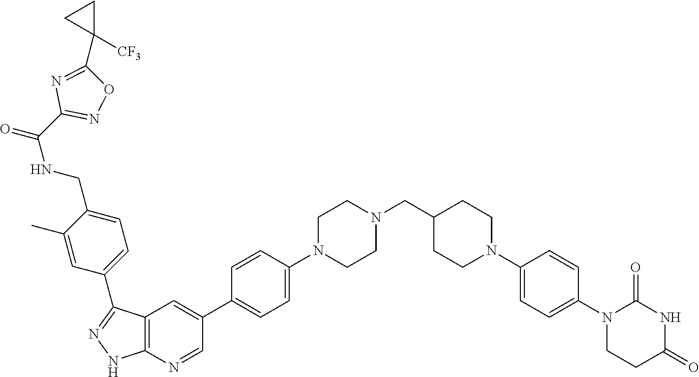 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1noc(C2(C(F)(F)F)CC2)n1]

The titled compound was synthesized in the procedures similar to Example 1. ¹H NMR (400 MHz, DMSO) δ_{H} 13.78 (s, 1H), 10.27 (s, 1H), 9.54 (s, 1H), 8.82 (s, 1H), 8.58 (s, 1H), 7.89 (s, 2H), 7.69 (d, J=7.6 Hz, 2H), 7.40 (d, J=7.8 Hz, 1H), 7.13 (d, J=8.2 Hz, 2H), 7.07 (d, J=7.8 Hz, 2H), 6.93 (d, J=8.0 Hz, 2H), 4.53 (s, 2H), 3.76-3.66 (m, 4H), 3.22 (s, 4H), 2.75-2.53 (m, 7H), 2.45 (s, 3H), 2.24 (s, 2H), 1.83 (s, 6H), 1.73 (s, 1H), 1.31-1.18 (m, 2H), 1.05 (t, J=6.9 Hz, 1H); [M+H]⁺=888.5.

### Example 43: (R)-5-(tert-butyl)-N-(1-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-3-fluoro-2-methylphenyl)ethyl)-1,2,4-oxadiazole-3-carboxamide

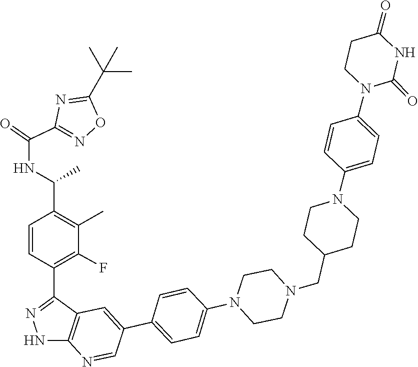 [SMILES: Cc1c([C@@H](C)NC(=O)c2noc(C(C)(C)C)n2)ccc(-c2n[nH]c3ncc(-c4ccc(N5CCN(CC6CCN(c7ccc(N8CCC(=O)NC8=O)cc7)CC6)CC5)cc4)cc23)c1F]

The titled compound was synthesized in the procedures similar to Example 1. ¹H NMR (400 MHz, DMSO) δ_{H} 13.96 (s, 1H), 10.27 (s, 1H), 9.58 (d, J=8.0 Hz, 1H), 8.84 (s, 1H), 8.32 (s, 1H), 7.71 (t, J=8.0 Hz, 1H), 7.62 (d, J=8.0 Hz, 2H), 7.45 (d, J=8.0 Hz, 1H), 7.13 (d, J=8.0 Hz, 2H), 7.06 (d, J=8.0 Hz, 2H), 6.94 (d, J=8.0 Hz, 2H), 5.38 (s, 1H), 3.69 (s, 4H), 3.21 (s, 4H), 2.74-2.62 (m, 4H), 2.54 (s, 2H), 2.40 (s, 3H), 2.24 (s, 2H), 1.81 (d, J=8.0 Hz, 2H), 1.73 (s, 1H), 1.52 (d, J=8.0 Hz, 3H), 1.43 (s, 9H), 1.24 (s, 4H); [M+H]⁺=868.9.

### Example 44: (R)-5-(tert-butyl)-N-(1-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-5-fluoro-2-methylphenyl)ethyl)-1,2,4-oxadiazole-3-carboxamide

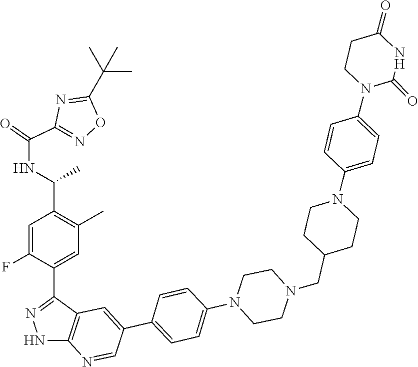 [SMILES: Cc1cc(-c2n[nH]c3ncc(-c4ccc(N5CCN(CC6CCN(c7ccc(N8CCC(=O)NC8=O)cc7)CC6)CC5)cc4)cc23)c(F)cc1[C@@H](C)NC(=O)c1noc(C(C)(C)C)n1]

The titled compound was synthesized in the procedures similar to Example 1. ¹H NMR (400 MHz, DMSO) δ_{H} 13.98 (s, 1H), 10.27 (s, 1H), 9.55 (d, J=8.0 Hz, 1H), 8.84 (s, 1H), 8.30 (s, 1H), 7.69 (d, J=4.0 Hz, 1H), 7.63 (d, J=8.0 Hz, 2H), 7.48 (d, J=12.0 Hz, 1H), 7.12 (d, J=8.0 Hz, 2H), 7.06 (d, J=8.0 Hz, 2H), 6.94 (d, J=8.0 Hz, 2H), 5.35 (s, 1H), 3.69 (s, 4H), 3.21 (s, 4H), 2.75-2.58 (m, 5H), 2.53 (s, 2H), 2.45 (s, 4H), 2.23 (d, J=4.0 Hz, 2H), 1.80 (s, 2H), 1.78-1.66 (m, 1H), 1.51 (d, J=4.0 Hz, 3H), 1.43 (s, 9H), 1.24 (s, 3H); [M+H]⁺=868.8.

### Example 45: (R)-3-(tert-butyl)-N-(1-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-3-fluoro-2-methylphenyl)ethyl)-1,2,4-oxadiazole-5-carboxamide

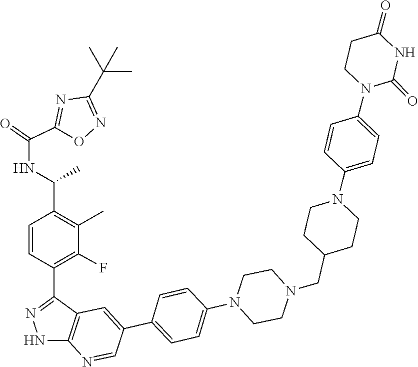 [SMILES: Cc1c([C@@H](C)NC(=O)c2nc(C(C)(C)C)no2)ccc(-c2n[nH]c3ncc(-c4ccc(N5CCN(CC6CCN(c7ccc(N8CCC(=O)NC8=O)cc7)CC6)CC5)cc4)cc23)c1F]

The titled compound was synthesized in the procedures similar to Example 1. ¹H NMR (400 MHz, DMSO) δ_{H} 13.96 (s, 1H), 10.27 (s, 1H), 9.98 (d, J=4.0 Hz, 2H), 8.84 (s, 1H), 8.31 (s, 1H), 7.72 (s, 1H), 7.64 (d, J=8.0 Hz, 2H), 7.47 (d, J=8.0 Hz, 1H), 7.13 (d, J=4.0 Hz, 2H), 7.07 (d, J=8.0 Hz, 2H), 6.94 (d, J=8.0 Hz, 2H), 5.37 (s, 1H), 3.70 (s, 4H), 3.21 (s, 4H), 2.67 (s, 5H), 2.40 (s, 3H), 2.23 (s, 2H), 1.81 (d, J=8.0 Hz, 2H), 1.72 (s, 1H), 1.55 (s, 3H), 1.37 (s, 9H), 1.23 (s, 3H); [M+H]⁺=868.8.

### Example 46: (R)-3-(tert-butyl)-N-(1-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-5-fluoro-2-methylphenyl)ethyl)-1,2,4-oxadiazole-5-carboxamide

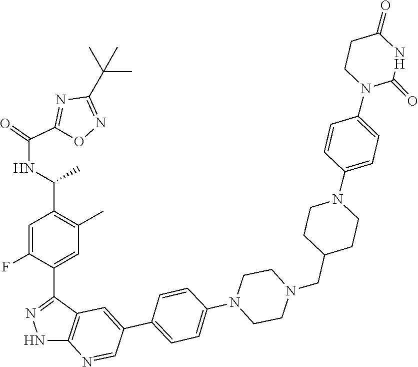 [SMILES: Cc1cc(-c2n[nH]c3ncc(-c4ccc(N5CCN(CC6CCN(c7ccc(N8CCC(=O)NC8=O)cc7)CC6)CC5)cc4)cc23)c(F)cc1[C@@H](C)NC(=O)c1nc(C(C)(C)C)no1]

The titled compound was synthesized in the procedures similar to Example 1. ¹H NMR (400 MHz, DMSO) δ_{H} 13.98 (s, 1H), 10.26 (s, 1H), 9.92 (d, J=8.0 Hz, 1H), 8.84 (s, 2H), 8.30 (s, 2H), 7.69 (d, J=4.0 Hz, 2H), 7.62 (d, J=8.0 Hz, 2H), 7.51 (d, J=12.0 Hz, 1H), 7.14 (d, J=8.0 Hz, 1H), 7.05 (d, J=8.0 Hz, 2H), 6.93 (d, J=8.0 Hz, 2H), 5.34 (s, 2H), 3.70 (s, 4H), 3.21 (s, 4H), 2.72-2.65 (m, 5H), 2.45 (s, 5H), 2.23 (d, J=4.0 Hz, 2H), 1.82 (d, J=12.0 Hz, 2H), 1.73 (s, 1H), 1.54 (d, J=4.0 Hz, 3H), 1.37 (s, 9H), 1.35-1.17 (m, 3H); [M+H]⁺=868.8.

### Example 47: 5-(tert-butyl)-N-(4-(5-(4-(4-(2-(1-(4-((2,6-dioxopiperidin-3-yl)amino)-3-methylphenyl)piperidin-4-yl)ethyl)piperazin-1-yl)phenyl)-1H-pyrazolo[3,4-b]pyridin-3-yl)-2-methylbenzyl)-1,2,4-oxadiazole-3-carboxamide

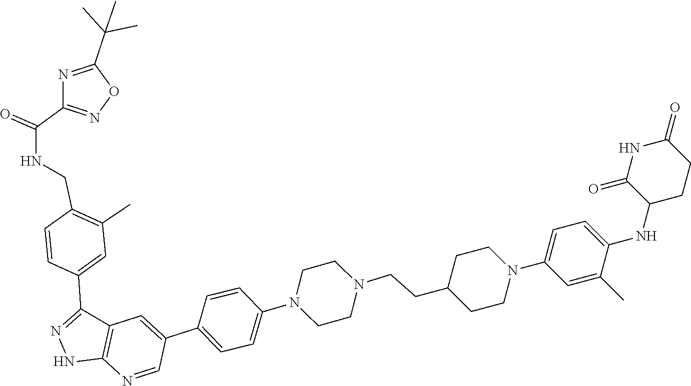 [SMILES: Cc1cc(-c2n[nH]c3ncc(-c4ccc(N5CCN(CCC6CCN(c7ccc(NC8CCC(=O)NC8=O)c(C)c7)CC6)CC5)cc4)cc23)ccc1CNC(=O)c1noc(C(C)(C)C)n1]

### Example 48: 5-(tert-butyl)-N-(4-(5-(4-(4-(2-(1-(4-(2,6-dioxopiperidin-3-yl)-3-fluorophenyl)piperidin-4-yl)ethyl)piperazin-1-yl)phenyl)-1H-pyrazolo[3,4-b]pyridin-3-yl)-2-methylbenzyl)-1,2,4-oxadiazole-3-carboxamide

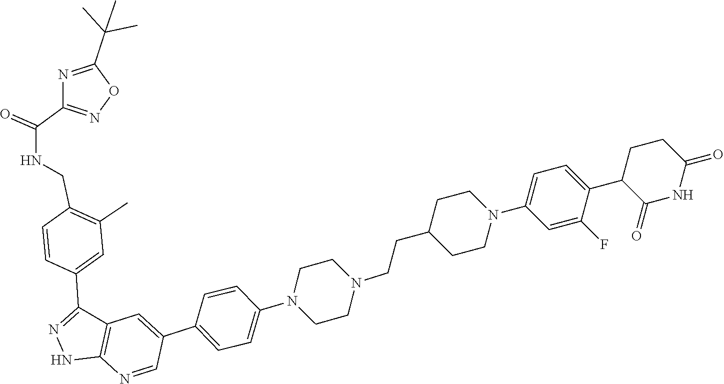 [SMILES: Cc1cc(-c2n[nH]c3ncc(-c4ccc(N5CCN(CCC6CCN(c7ccc(C8CCC(=O)NC8=O)c(F)c7)CC6)CC5)cc4)cc23)ccc1CNC(=O)c1noc(C(C)(C)C)n1]

The titled compound was synthesized in the procedures similar to Example 1. ¹H NMR (400 MHz, DMSO) δ_{H} 13.78 (s, 1H), 10.81 (s, 1H), 9.46 (s, 1H), 8.82 (s, 1H), 8.58 (s, 1H), 7.94-7.85 (m, 2H), 7.69 (d, J=8.0 Hz, 2H), 7.40 (d, J=4.0 Hz, 1H), 7.10-7.00 (m, 3H), 6.71 (d, J=8.0 Hz, 2H), 4.52 (d, J=4.0 Hz, 2H), 3.86 (d, J=4.0 Hz, 1H), 3.71 (d, J=12.0 Hz, 2H), 3.21 (s, 4H), 2.69 (t, J=12.0 Hz, 4H), 2.47-2.35 (m, 7H), 2.18-2.08 (m, 1H), 2.00-1.90 (m, 1H), 1.76 (d, J=12.0 Hz, 2H), 1.55-1.39 (m, 14H), 1.29-1.18 (m, 3H); [M+H]⁺=867.9.

### Example 49: 5-(tert-butyl)-N-(4-(5-(4-(4-(2-(1-(4-((2,6-dioxopiperidin-3-yl)amino)-3-fluorophenyl)piperidin-4-yl)ethyl)piperazin-1-yl)phenyl)-1H-pyrazolo[3,4-b]pyridin-3-yl)-2-methylbenzyl)-1,2,4-oxadiazole-3-carboxamide

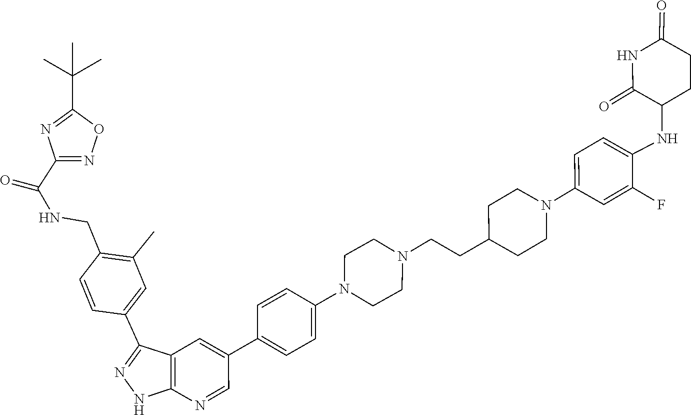 [SMILES: Cc1cc(-c2n[nH]c3ncc(-c4ccc(N5CCN(CCC6CCN(c7ccc(NC8CCC(=O)NC8=O)c(F)c7)CC6)CC5)cc4)cc23)ccc1CNC(=O)c1noc(C(C)(C)C)n1]

### Example 50: 5-(tert-butyl)-N-(4-(5-(4-(4-((1-(4-(2,4-dioxotetrahydropyrimidin-1(2H)-yl)-3-methoxyphenyl)piperidin-4-yl)methyl)piperazin-1-yl)phenyl)-1H-pyrazolo[3,4-b]pyridin-3-yl)-2-methylbenzyl)-1,2,4-oxadiazole-3-carboxamide

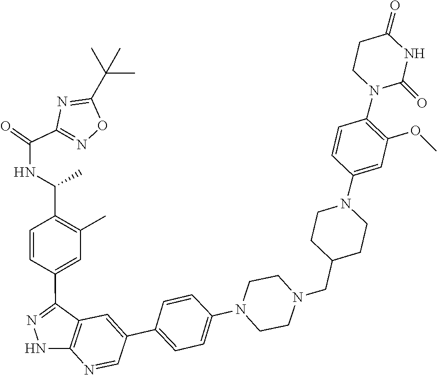 [SMILES: COc1cc(N2CCC(CN3CCN(c4ccc(-c5cnc6[nH]nc(-c7ccc([C@@H](C)NC(=O)c8noc(C(C)(C)C)n8)c(C)c7)c6c5)cc4)CC3)CC2)ccc1N1CCC(=O)NC1=O]

The titled compound was synthesized in the procedures similar to Example 1. ¹H NMR (400 MHz, DMSO) δ_{H} 13.78 (s, 1H), 10.21 (s, 1H), 9.46 (s, 1H), 8.82 (s, 1H), 8.58 (s, 1H), 7.95-7.84 (m, 3H), 7.68 (d, J=8.0 Hz, 2H), 7.41 (d, J=8.0 Hz, 1H), 7.08 (d, J=8.0 Hz, 2H), 7.01 (d, J=8.0 Hz, 1H), 6.59 (s, 1H), 6.50 (d, J=4.0 Hz, 2H), 4.52 (s, 2H), 3.80-3.70 (m, 5H), 3.50 (s, 2H), 3.22 (s, 4H), 2.77-2.58 (m, 6H), 2.45 (s, 4H), 2.27-2.20 (m, 2H), 1.81 (d, J=8.0, 2H), 1.78-1.70 (m, 1H), 1.44 (s, 9H), 1.32-1.16 (m, 3H); [M+H]⁺=866.8.

### Example 51: 5-(tert-butyl)-N-(4-(5-(4-(4-(3-chloro-4-(2,6-dioxopiperidin-3-yl)phenethyl)piperazin-1-yl)phenyl)-1H-pyrazolo[3,4-b]pyridin-3-yl)-2-methylbenzyl)-1,2,4-oxadiazole-3-carboxamide

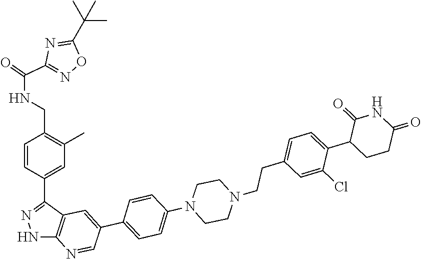 [SMILES: Cc1cc(-c2n[nH]c3ncc(-c4ccc(N5CCN(CCc6ccc(C7CCC(=O)NC7=O)c(Cl)c6)CC5)cc4)cc23)ccc1CNC(=O)c1noc(C(C)(C)C)n1]

The titled compound was synthesized in the procedures similar to Example 1. ¹H NMR (400 MHz, DMSO) δ_{H} 13.78 (s, 1H), 10.90 (s, 1H), 9.46 (s, 1H), 8.82 (s, 1H), 8.58 (s, 1H), 7.94-7.86 (m, 2H), 7.69 (d, J=8.0 Hz, 3H), 7.43-7.37 (m, 2H), 7.24 (s, 2H), 7.08 (d, J=4.0 Hz, 2H), 4.52 (d, J=4.0 Hz, 2H), 4.17 (d, J=8.0 Hz, 1H), 3.30 (s, 1H), 3.22 (s, 4H), 2.80 (s, 3H), 2.75-2.70 (m, 1H), 2.62 (s, 5H), 2.45-2.43 (m, 3H), 2.31-2.24 (m, 1H), 1.99-1.92 (m, 1H), 1.44 (s, 9H); [M+H]⁺=800.7.

### Example 52: 5-(tert-butyl)-N-(4-(5-(4-(4-(3-(4-(2,4-dioxotetrahydropyrimidin-1(2H)-yl)-3-methylphenoxy)propyl)piperazin-1-yl)phenyl)-1H-pyrazolo[3,4-b]pyridin-3-yl)-2-methylbenzyl)-1,2,4-oxadiazole-3-carboxamide

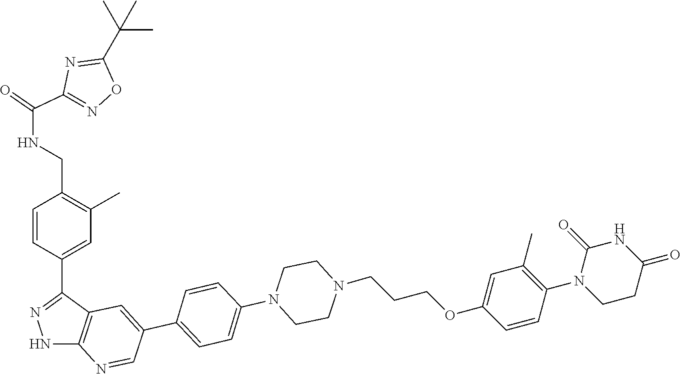 [SMILES: Cc1cc(-c2n[nH]c3ncc(-c4ccc(N5CCN(CCCOc6ccc(N7CCC(=O)NC7=O)c(C)c6)CC5)cc4)cc23)ccc1CNC(=O)c1noc(C(C)(C)C)n1]

### Step 1: 3-(4-(2,4-dioxotetrahydropyrimidin-1(2H)-yl)-3-methylphenoxy)propyl methanesulfonate

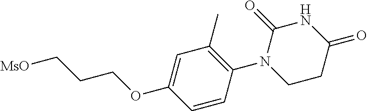 [SMILES: Cc1cc(OCCCOS(C)(=O)=O)ccc1N1CCC(=O)NC1=O]

To a solution of 1-(4-(3-hydroxypropoxy)-2-methylphenyl)dihydropyrimidine-2,4(1H,3H)-dione (500 mg, 1.799 mmol) and TEA (272.5 mg, 2.699 mmol) in THF (10 mL), DMF (1 mL) was added. Then MsCl (246 mg, 2.158 mmol) was added slowly at 0° C.˜5° C. The mixture was stirred at room temperature overnight. After the reaction was completed determined by LCMS, the mixture was extracted with EtOAc, washed with brine, dried over Na₂SO₄, and concentrated in vacuo to give the desired product (480 mg, 75%). [M+H]⁺=357.3.

### Step 2: 5-(tert-butyl)-N-(4-(5-(4-(4-(3-(4-(2,4-dioxotetrahydropyrimidin-1(2H)-yl)-3-methylphenoxy)propyl)piperazin-1-yl)phenyl)-1H-pyrazolo[3,4-b]pyridin-3-yl)-2-methylbenzyl)-1,2,4-oxadiazole-3-carboxamide

To a solution of 5-(tert-butyl)-N-(2-methyl-4-(5-(4-(piperazin-1-yl)phenyl)-1H-pyrazolo[3,4-b]pyridin-3-yl)benzyl)-1,2,4-oxadiazole-3-carboxamide (120 mg, 0.218 mmol), KI (108 mg, 0.654 mmol) and TEA (110 mg, 1.09 mmol) in DMF (5 mL), 3-(4-(2,4-dioxotetrahydropyrimidin-1(2H)-yl)-3-methylphenoxy)propyl methanesulfonate (93 mg, 0.262 mg) was added dropwise at 0° C.˜5° C. The mixture was stirred at 65° C. overnight. After the reaction was completed determined by LCMS, the mixture was concentrated in vacuo. The residue was purified by silica gel column chromatography DCM:MeOH=94%:6% to give the desired product (19.92 mg, 11%). ¹H NMR (400 MHz, DMSO) δ_{H} 13.78 (s, 1H), 10.29 (s, 1H), 9.46 (s, 1H), 8.82 (s, 1H), 8.58 (s, 1H), 7.94-7.82 (m, 2H), 7.69 (d, J=8.0 Hz, 2H), 7.40 (d, J=8.0 Hz, 1H), 7.16 (d, J=8.0 Hz, 1H), 7.08 (d, J=8.0 Hz, 2H), 6.86 (s, 1H), 6.81 (d, J=8.0 Hz, 1H), 4.51 (d, J=4.0 Hz, 1H), 4.04 (s, 2H), 3.70 (s, 1H), 3.51-3.43 (m, 1H), 3.22 (s, 4H), 2.77-2.66 (m, 3H), 2.63-2.56 (m, 3H), 2.45 (s, 5H), 2.15 (s, 3H), 1.94 (s, 2H), 1.44 (s, 9H); [M+H]⁺=811.8.

### Example 53: 3-(tert-butyl)-N-(4-(5-(4-(1-(3-(4-(2,4-dioxotetrahydropyrimidin-1(2H)-yl)phenoxy)propyl)piperidin-4-yl)phenyl)-1H-pyrazolo[3,4-b]pyridin-3-yl)-2-fluorobenzyl)-1,2,4-oxadiazole-5-carboxamide

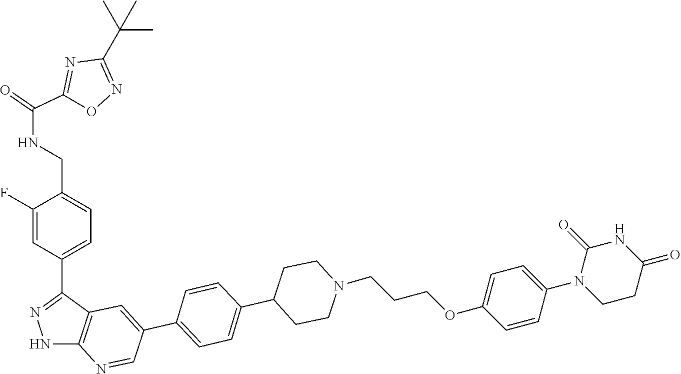 [SMILES: CC(C)(C)c1noc(C(=O)NCc2ccc(-c3n[nH]c4ncc(-c5ccc(C6CCN(CCCOc7ccc(N8CCC(=O)NC8=O)cc7)CC6)cc5)cc34)cc2F)n1]

### Example 54: 3-(tert-butyl)-N-(4-(5-(4-(1-(3-(4-(2,6-dioxopiperidin-3-yl)phenoxy)propyl)piperidin-4-yl)phenyl)-1H-pyrazolo[3,4-b]pyridin-3-yl)-2-fluorobenzyl)-1,2,4-oxadiazole-5-carboxamide

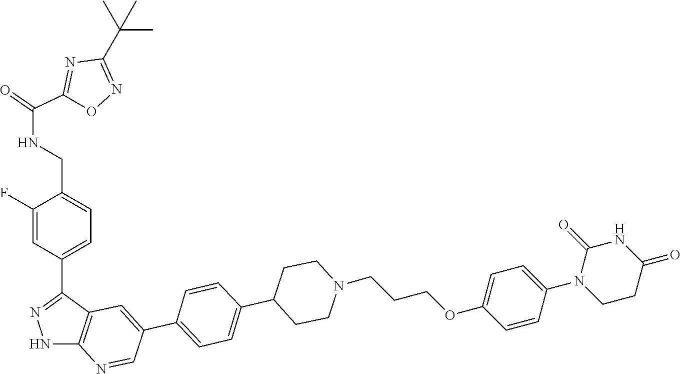 [SMILES: CC(C)(C)c1noc(C(=O)NCc2ccc(-c3n[nH]c4ncc(-c5ccc(C6CCN(CCCOc7ccc(N8CCC(=O)NC8=O)cc7)CC6)cc5)cc34)cc2F)n1]

### Example 55: 3-(tert-butyl)-N-(4-(5-(4-(4-(3-(4-(2,4-dioxotetrahydropyrimidin-1(2H)-yl)phenoxy)propyl)piperazin-1-yl)phenyl)-1H-pyrazolo[3,4-b]pyridin-3-yl)-2-fluorobenzyl)-1,2,4-oxadiazole-5-carboxamide

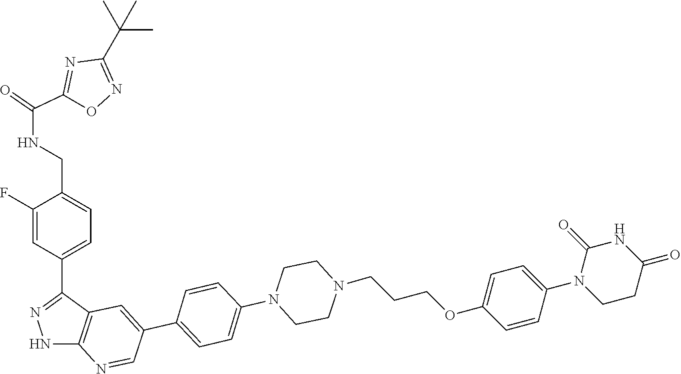 [SMILES: CC(C)(C)c1noc(C(=O)NCc2ccc(-c3n[nH]c4ncc(-c5ccc(N6CCN(CCCOc7ccc(N8CCC(=O)NC8=O)cc7)CC6)cc5)cc34)cc2F)n1]

### Example 56: 3-(tert-butyl)-N-(4-(5-(6-(1-(3-(4-(2,4-dioxotetrahydropyrimidin-1(2H)-yl)phenoxy)propyl)piperidin-4-yl)pyridin-3-yl)-1H-pyrazolo[3,4-b]pyridin-3-yl)-2-fluorobenzyl)-1,2,4-oxadiazole-5-carboxamide

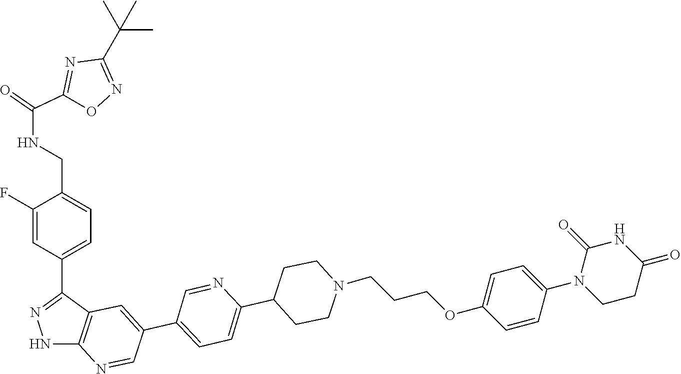 [SMILES: CC(C)(C)c1noc(C(=O)NCc2ccc(-c3n[nH]c4ncc(-c5ccc(C6CCN(CCCOc7ccc(N8CCC(=O)NC8=O)cc7)CC6)nc5)cc34)cc2F)n1]

### Example 57: 3-(tert-butyl)-N-(4-(5-(5-(1-(3-(4-(2,4-dioxotetrahydropyrimidin-1(2H)-yl)phenoxy)propyl)piperidin-4-yl)pyridin-2-yl)-1H-pyrazolo[3,4-b]pyridin-3-yl)-2-fluorobenzyl)-1,2,4-oxadiazole-5-carboxamide

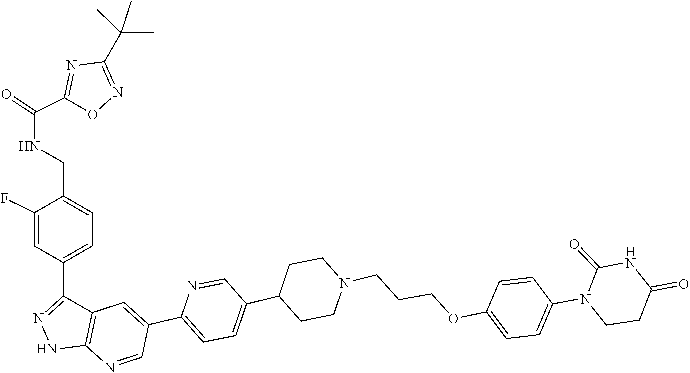 [SMILES: CC(C)(C)c1noc(C(=O)NCc2ccc(-c3n[nH]c4ncc(-c5ccc(C6CCN(CCCOc7ccc(N8CCC(=O)NC8=O)cc7)CC6)cn5)cc34)cc2F)n1]

### Example 58: 3-(tert-butyl)-N-(4-(5-(4-(4-(4-(2,4-dioxotetrahydropyrimidin-1(2H)-yl)phenethyl)piperazin-1-yl)phenyl)-1H-pyrazolo[3,4-b]pyridin-3-yl)-2-fluorobenzyl)-1,2,4-oxadiazole-5-carboxamide

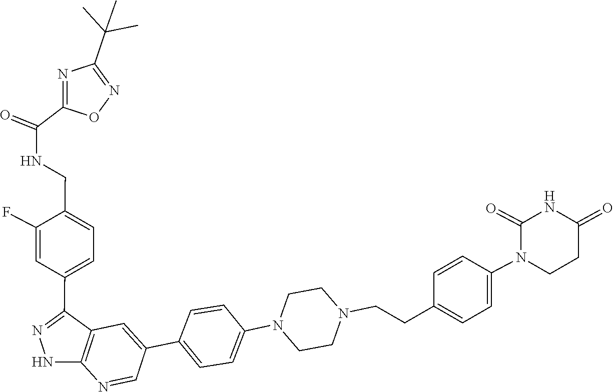 [SMILES: CC(C)(C)c1noc(C(=O)NCc2ccc(-c3n[nH]c4ncc(-c5ccc(N6CCN(CCc7ccc(N8CCC(=O)NC8=O)cc7)CC6)cc5)cc34)cc2F)n1]

### Example 59: 3-(tert-butyl)-N-(4-(5-(4-(4-(2-(1-(4-(2,4-dioxotetrahydropyrimidin-1(2H)-yl)phenyl)piperidin-4-yl)ethyl)piperazin-1-yl)phenyl)-1H-pyrazolo[3,4-b]pyridin-3-yl)-2-fluorobenzyl)-1,2,4-oxadiazole-5-carboxamide

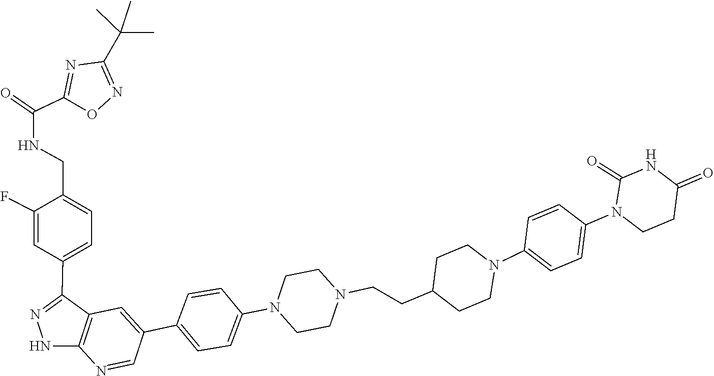 [SMILES: CC(C)(C)c1noc(C(=O)NCc2ccc(-c3n[nH]c4ncc(-c5ccc(N6CCN(CCC7CCN(c8ccc(N9CCC(=O)NC9=O)cc8)CC7)CC6)cc5)cc34)cc2F)n1]

### Example 60: (S)-3-(tert-butyl)-N-(4-(5-(4-(4-(2-(1-(4-(2,4-dioxotetrahydropyrimidin-1(2H)-yl)phenyl)piperidin-4-yl)ethyl)-2-methylpiperazin-1-yl)phenyl)-1H-pyrazolo[3,4-b]pyridin-3-yl)-2-fluorobenzyl)-1,2,4-oxadiazole-5-carboxamide

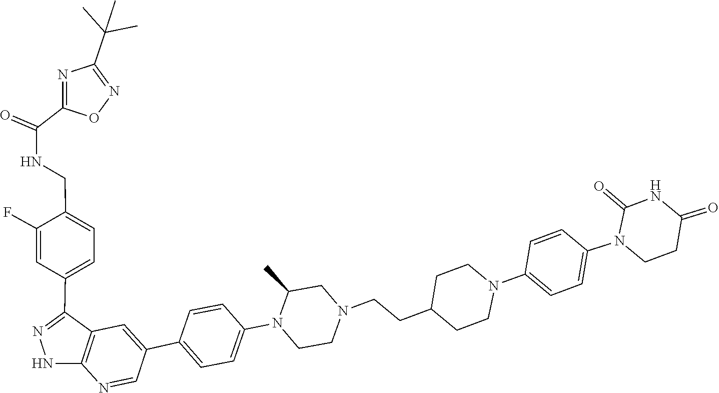 [SMILES: C[C@H]1CN(CCC2CCN(c3ccc(N4CCC(=O)NC4=O)cc3)CC2)CCN1c1ccc(-c2cnc3[nH]nc(-c4ccc(CNC(=O)c5nc(C(C)(C)C)no5)c(F)c4)c3c2)cc1]

### Example 61: 3-(tert-butyl)-N-(4-(5-(6-(4-(2-(1-(4-(2,4-dioxotetrahydropyrimidin-1(2H)-yl)phenyl)piperidin-4-yl)ethyl)piperazin-1-yl)pyridin-3-yl)-1H-pyrazolo[3,4-b]pyridin-3-yl)-2-fluorobenzyl)-1,2,4-oxadiazole-5-carboxamide

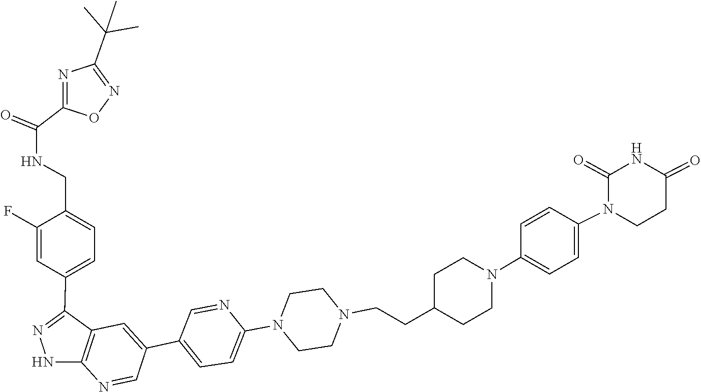 [SMILES: CC(C)(C)c1noc(C(=O)NCc2ccc(-c3n[nH]c4ncc(-c5ccc(N6CCN(CCC7CCN(c8ccc(N9CCC(=O)NC9=O)cc8)CC7)CC6)nc5)cc34)cc2F)n1]

### Example 62: 3-(tert-butyl)-N-(4-(5-(5-(4-(2-(1-(4-(2,4-dioxotetrahydropyrimidin-1(2H)-yl)phenyl)piperidin-4-yl)ethyl)piperazin-1-yl)pyridin-2-yl)-1H-pyrazolo[3,4-b]pyridin-3-yl)-2-fluorobenzyl)-1,2,4-oxadiazole-5-carboxamide

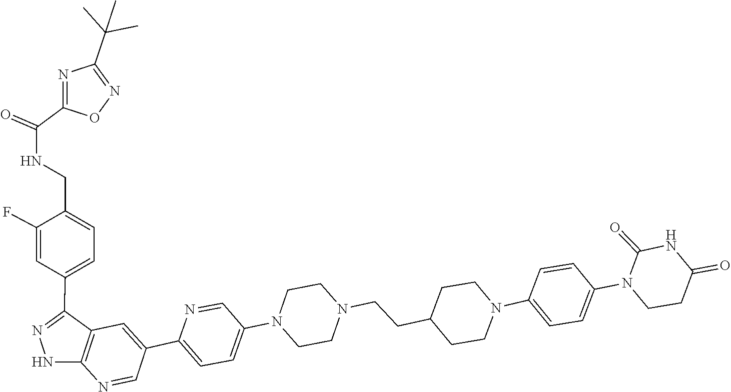 [SMILES: CC(C)(C)c1noc(C(=O)NCc2ccc(-c3n[nH]c4ncc(-c5ccc(N6CCN(CCC7CCN(c8ccc(N9CCC(=O)NC9=O)cc8)CC7)CC6)cn5)cc34)cc2F)n1]

### Example 63: 5-(tert-butyl)-N-(4-(5-(6-(1-((1-(4-(2,4-dioxotetrahydropyrimidin-1(2H)-yl)phenyl)piperidin-4-yl)methyl)piperidin-4-yl)pyridin-3-yl)-1H-pyrazolo[3,4-b]pyridin-3-yl)-2-methylbenzyl)-1,2,4-oxadiazole-3-carboxamide

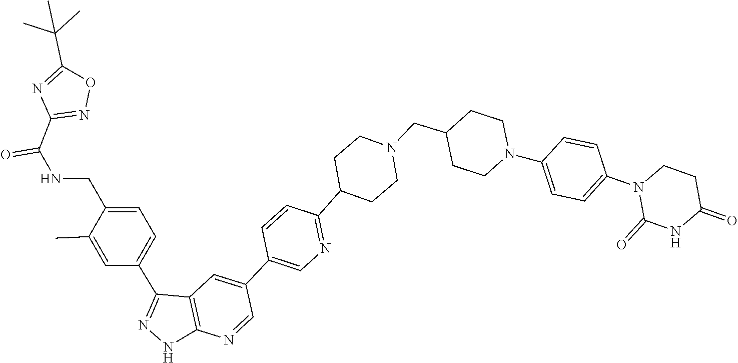 [SMILES: Cc1cc(-c2n[nH]c3ncc(-c4ccc(C5CCN(CC6CCN(c7ccc(N8CCC(=O)NC8=O)cc7)CC6)CC5)nc4)cc23)ccc1CNC(=O)c1noc(C(C)(C)C)n1]

### Example 64: 5-(tert-butyl)-N-(4-(5-(5-(1-((1-(4-(2,4-dioxotetrahydropyrimidin-1(2H)-yl)phenyl)piperidin-4-yl)methyl)piperidin-4-yl)pyridin-2-yl)-1H-pyrazolo[3,4-b]pyridin-3-yl)-2-methylbenzyl)-1,2,4-oxadiazole-3-carboxamide

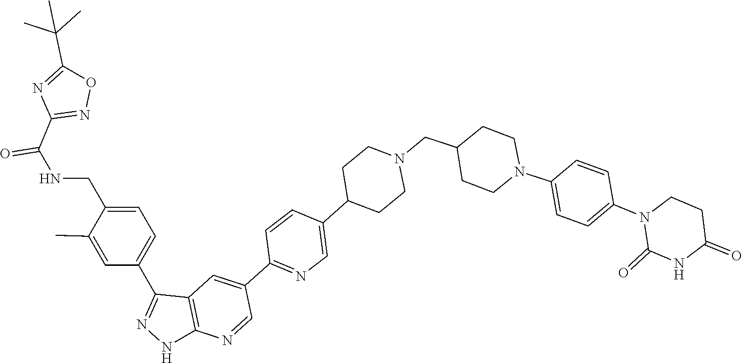 [SMILES: Cc1cc(-c2n[nH]c3ncc(-c4ccc(C5CCN(CC6CCN(c7ccc(N8CCC(=O)NC8=O)cc7)CC6)CC5)cn4)cc23)ccc1CNC(=O)c1noc(C(C)(C)C)n1]

### Example 65: 5-(tert-butyl)-N-(4-(5-(6-(4-((1-(4-(2,4-dioxotetrahydropyrimidin-1(2H)-yl)phenyl)piperidin-4-yl)methyl)piperazin-1-yl)pyridin-3-yl)-1H-pyrazolo[3,4-b]pyridin-3-yl)-2-methylbenzyl)-1,2,4-oxadiazole-3-carboxamide

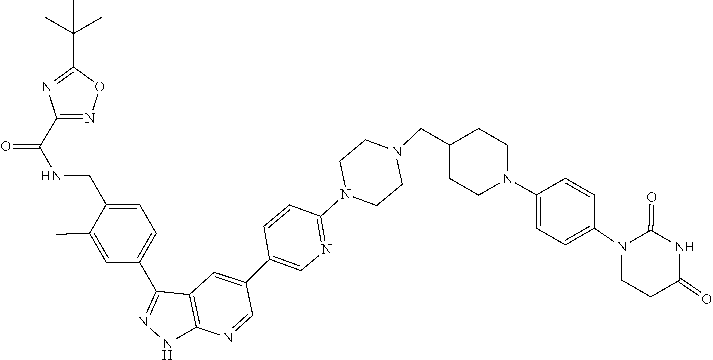 [SMILES: Cc1cc(-c2n[nH]c3ncc(-c4ccc(N5CCN(CC6CCN(c7ccc(N8CCC(=O)NC8=O)cc7)CC6)CC5)nc4)cc23)ccc1CNC(=O)c1noc(C(C)(C)C)n1]

The titled compound was synthesized in the procedures similar to Example 1. ¹H NMR (400 MHz, CDCl₃) δ_{H} 13.82 (s, 1H), 10.27 (s, 1H), 9.47 (s, 1H), 8.83 (s, 1H), 8.65-8.59 (m, 2H), 8.04-7.90 (m, 3H), 7.39 (d, J=8.0 Hz, 1H), 7.14 (d, J=8.0 Hz, 1H), 6.97-6.92 (m, 3H), 4.52 (s, 2H), 3.70-3.56 (m, 8H), 2.68-2.66 (m, 4H), 2.49-2.45 (m, 6H), 2.23 (s, 2H), 1.84-1.73 (m, 3H), 1.43 (s, 9H), 1.25-1.23 (m, 3H); [M+H]⁺=836.8.

### Example 66: 5-(tert-butyl)-N-(4-(5-(5-(4-((1-(4-(2,4-dioxotetrahydropyrimidin-1(2H)-yl)phenyl)piperidin-4-yl)methyl)piperazin-1-yl)pyridin-2-yl)-1H-pyrazolo[3,4-b]pyridin-3-yl)-2-methylbenzyl)-1,2,4-oxadiazole-3-carboxamide

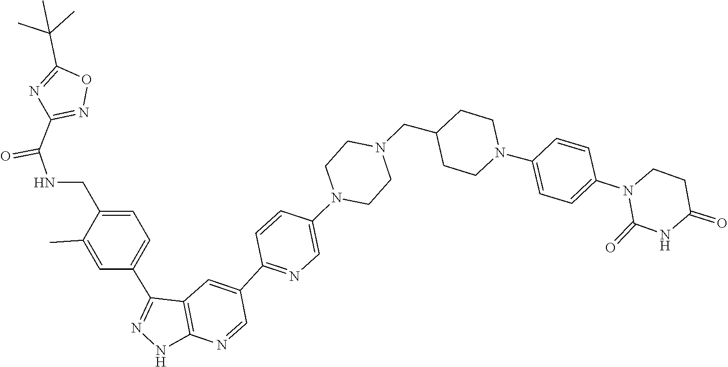 [SMILES: Cc1cc(-c2n[nH]c3ncc(-c4ccc(N5CCN(CC6CCN(c7ccc(N8CCC(=O)NC8=O)cc7)CC6)CC5)cn4)cc23)ccc1CNC(=O)c1noc(C(C)(C)C)n1]

The titled compound was synthesized in the procedures similar to Example 1. ¹H NMR (400 MHz, DMSO) δ_{H} 13.83 (s, 1H), 10.27 (s, 1H), 9.47 (s, 1H), 9.23 (s, 1H), 8.94 (s, 1H), 8.45 (s, 1H), 8.04 (d, J=8.0 Hz, 1H), 7.93-7.87 (m, 2H), 7.45-7.43 (m, 2H), 7.14 (d, J=8.0 Hz, 2H), 6.93 (d, J=8.0 Hz, 2H), 4.53 (s, 2H), 3.70-3.68 (m, 4H), 3.29-3.27 (m, 4H), 2.69-2.68 (m, 4H), 2.58-2.56 (m, 4H), 2.51 (s, 3H), 2.24 (s, 2H), 1.84-1.74 (m, 3H), 1.44 (s, 9H), 1.25-1.23 (m, 2H); [M+H]⁺=836.8.

### Example 67: 3-(tert-butyl)-N-(4-(5-(5-(4-((1-(4-(2,4-dioxotetrahydropyrimidin-1(2H)-yl)phenyl)piperidin-4-yl)methyl)piperazin-1-yl)pyridin-2-yl)-1H-pyrazolo[3,4-b]pyridin-3-yl)-2-methylbenzyl)-1,2,4-oxadiazole-5-carboxamide

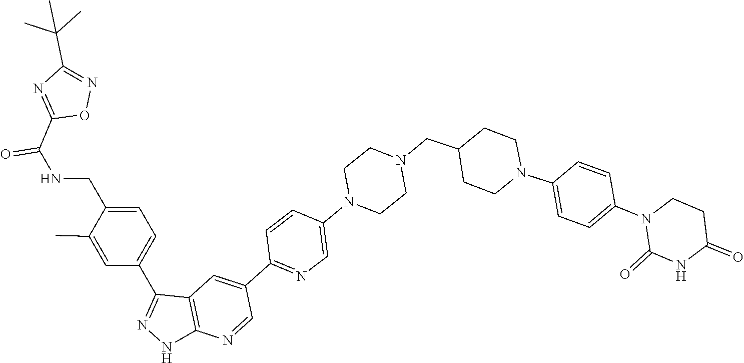 [SMILES: Cc1cc(-c2n[nH]c3ncc(-c4ccc(N5CCN(CC6CCN(c7ccc(N8CCC(=O)NC8=O)cc7)CC6)CC5)cn4)cc23)ccc1CNC(=O)c1nc(C(C)(C)C)no1]

### Example 68: 3-(tert-butyl)-N-(4-(5-(6-(4-((1-(4-(2,4-dioxotetrahydropyrimidin-1(2H)-yl)phenyl)piperidin-4-yl)methyl)piperazin-1-yl)pyridin-3-yl)-1H-pyrazolo[3,4-b]pyridin-3-yl)-2-methylbenzyl)-1,2,4-oxadiazole-5-carboxamide

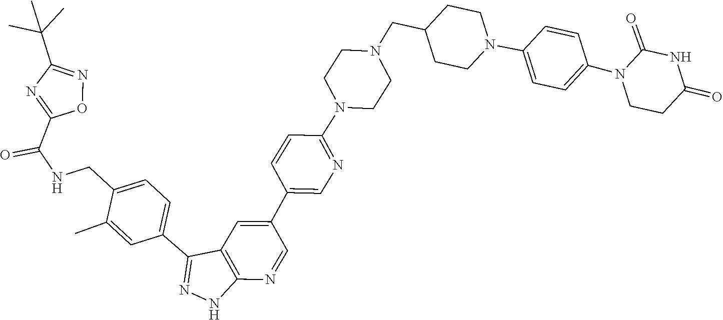 [SMILES: Cc1cc(-c2n[nH]c3ncc(-c4ccc(N5CCN(CC6CCN(c7ccc(N8CCC(=O)NC8=O)cc7)CC6)CC5)nc4)cc23)ccc1CNC(=O)c1nc(C(C)(C)C)no1]

The titled compound was synthesized in the procedures similar to Example 1. ¹H NMR (400 MHz, CDCl₃) δ_{H} 13.82 (s, 1H), 10.27 (s, 1H), 9.83 (s, 1H), 8.83 (s, 1H), 8.65 (s, 1H), 8.60 (s, 1H), 8.03 (d, J=8.0 Hz, 1H), 7.93-7.91 (m, 2H), 7.42 (d, J=8.0 Hz, 1H), 7.14 (d, J=8.0 Hz, 2H), 6.98-6.92 (m, 3H), 4.53 (s, 2H), 3.70-3.57 (m, 8H), 2.69-2.68 (m, 4H), 2.54 (s, 3H), 2.48-2.46 (m, 4H), 2.24 (s, 2H), 1.84-1.74 (m, 3H), 1.43 (s, 9H), 1.25-1.23 (m, 3H); [M+H]⁺=836.8.

### Example 69: 5-(tert-butyl)-N-(4-(5-(6-(4-((1-(4-(2,4-dioxotetrahydropyrimidin-1(2H)-yl)-3-methylphenyl)piperidin-4-yl)methyl)piperazin-1-yl)pyridin-3-yl)-1H-pyrazolo[3,4-b]pyridin-3-yl)-2-methylbenzyl)-1,2,4-oxadiazole-3-carboxamide

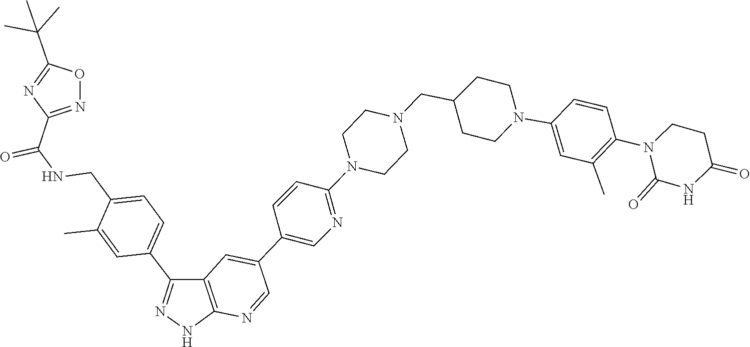 [SMILES: Cc1cc(-c2n[nH]c3ncc(-c4ccc(N5CCN(CC6CCN(c7ccc(N8CCC(=O)NC8=O)c(C)c7)CC6)CC5)nc4)cc23)ccc1CNC(=O)c1noc(C(C)(C)C)n1]

The titled compound was synthesized in the procedures similar to Example 1. ¹H NMR (400 MHz, DMSO) δ_{H} 13.81 (s, 1H), 10.25 (s, 1H), 9.47 (s, 1H), 8.83 (s, 1H), 8.65 (s, 1H), 8.60 (s, 1H), 8.03 (d, J=8.0 Hz, 1H), 7.94-7.91 (m, 2H), 7.40 (d, J=8.0 Hz, 1H), 7.06-6.95 (m, 2H), 6.82-6.77 (m, 2H), 4.52 (s, 2H), 3.71-3.69 (m, 3H), 3.57-3.48 (m, 5m), 2.70-2.68 (m, 5H), 2.54-2.33 (m, 5H), 2.28-2.24 (m, 2H), 2.12 (s, 3H), 1.84-1.74 (m, 4H), 1.44 (s, 9H), 1.25-1.23 (m, 2H); [M+H]⁺=851.8.

### Example 70: 5-(tert-butyl)-N-(4-(5-(5-(4-((1-(4-(2,4-dioxotetrahydropyrimidin-1(2H)-yl)-3-methylphenyl)piperidin-4-yl)methyl)piperazin-1-yl)pyridin-2-yl)-1H-pyrazolo[3,4-b]pyridin-3-yl)-2-methylbenzyl)-1,2,4-oxadiazole-3-carboxamide

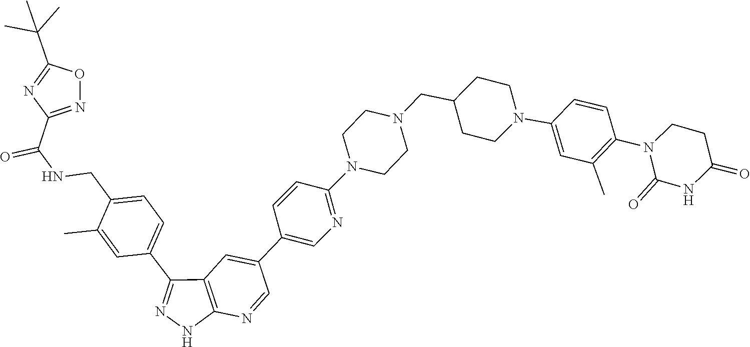 [SMILES: Cc1cc(-c2n[nH]c3ncc(-c4ccc(N5CCN(CC6CCN(c7ccc(N8CCC(=O)NC8=O)c(C)c7)CC6)CC5)nc4)cc23)ccc1CNC(=O)c1noc(C(C)(C)C)n1]

### Example 71: (R)-5-(tert-butyl)-N-(1-(4-(5-(6-(1-((1-(4-(2,4-dioxotetrahydropyrimidin-1(2H)-yl)phenyl)piperidin-4-yl)methyl)piperidin-4-yl)pyridin-3-yl)-1H-pyrazolo[3,4-b]pyridin-3-yl)-2-methylphenyl)ethyl)-1,2,4-oxadiazole-3-carboxamide

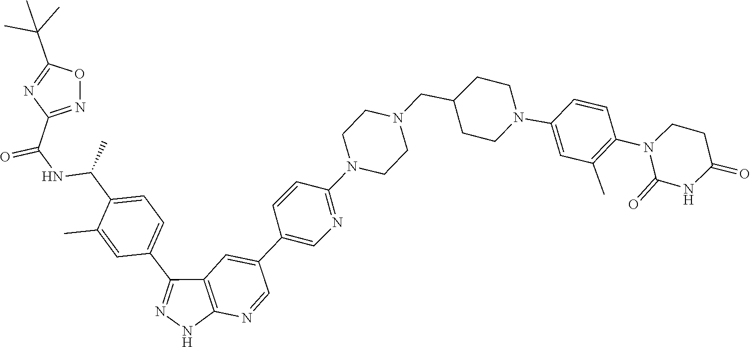 [SMILES: Cc1cc(-c2n[nH]c3ncc(-c4ccc(N5CCN(CC6CCN(c7ccc(N8CCC(=O)NC8=O)c(C)c7)CC6)CC5)nc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.90 (s, 1H), 10.25 (s, 1H), 9.48 (d, J=8.0 Hz, 1H), 9.00 (s, 1H), 8.90 (s, 1H), 8.79 (s, 1H), 8.22 (s, 1H), 8.01-7.83 (m, 3H), 7.60 (d, J=8.0 Hz, 2H), 7.44 (d, J=4.0 Hz, 1H), 7.15 (d, J=8.0 Hz, 2H), 6.95 (d, J=8.0 Hz, 2H), 5.36 (s, 1H), 3.82-3.58 (m, 5H), 3.20-2.82 (m, 5H), 2.77-2.65 (m, 4H), 2.25-1.96 (m, 5H), 1.87 (s, 4H), 1.51 (d, J=8.0 Hz, 3H), 1.42 (s, 9H), 1.34 (s, 2H), 1.23 (s, 2H); [M+H]⁺=850.8.

### Example 72: (R)-5-(tert-butyl)-N-(1-(4-(5-(5-(1-((1-(4-(2,4-dioxotetrahydropyrimidin-1(2H)-yl)phenyl)piperidin-4-yl)methyl)piperidin-4-yl)pyridin-2-yl)-1H-pyrazolo[3,4-b]pyridin-3-yl)-2-methylphenyl)ethyl)-1,2,4-oxadiazole-3-carboxamide

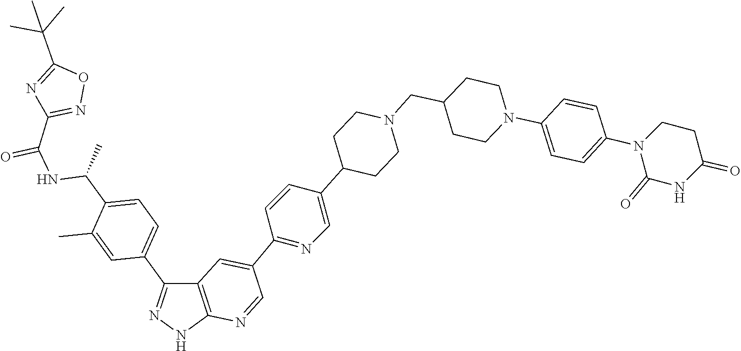 [SMILES: Cc1cc(-c2n[nH]c3ncc(-c4ccc(C5CCN(CC6CCN(c7ccc(N8CCC(=O)NC8=O)cc7)CC6)CC5)cn4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.89 (s, 1H), 10.25 (s, 1H), 9.47 (d, J=8.0 Hz, 1H), 9.28 (s, 1H), 9.02 (s, 1H), 8.63 (s, 1H), 8.15 (s, 1H), 7.94-7.76 (m, 3H), 7.63 (d, J=8.0 Hz, 1H), 7.13 (d, J=8.0 Hz, 2H), 6.95 (d, J=4.0 Hz, 2H), 5.37 (s, 1H), 3.70 (s, 4H), 3.00 (s, 4H), 2.77-2.59 (m, 5H), 2.30-1.98 (m, 5H), 1.95-1.67 (m, 6H), 1.52 (d, J=8.0 Hz, 3H), 1.43 (s, 9H), 1.23 (s, 3H); [M+H]⁺=850.8.

### Example 73: (R)-5-(tert-butyl)-N-(1-(4-(5-(6-(4-((1-(4-(2,4-dioxotetrahydropyrimidin-1(2H)-yl)phenyl)piperidin-4-yl)methyl)piperazin-1-yl)pyridin-3-yl)-1H-pyrazolo[3,4-b]pyridin-3-yl)-2-methylphenyl)ethyl)-1,2,4-oxadiazole-3-carboxamide

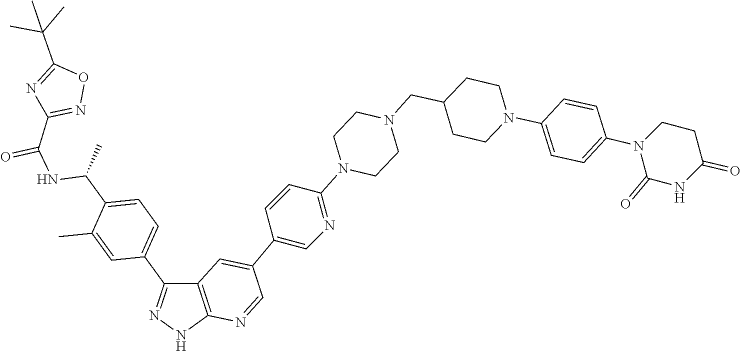 [SMILES: Cc1cc(-c2n[nH]c3ncc(-c4ccc(N5CCN(CC6CCN(c7ccc(N8CCC(=O)NC8=O)cc7)CC6)CC5)nc4)cc23)ccc1[C@@H](C)NC(=O)c1noc(C(C)(C)C)n1]

### Example 74: (R)-5-(tert-butyl)-N-(1-(4-(5-(5-(4-((1-(4-(2,4-dioxotetrahydropyrimidin-1(2H)-yl)phenyl)piperidin-4-yl)methyl)piperazin-1-yl)pyridin-2-yl)-1H-pyrazolo[3,4-b]pyridin-3-yl)-2-methylphenyl)ethyl)-1,2,4-oxadiazole-3-carboxamide

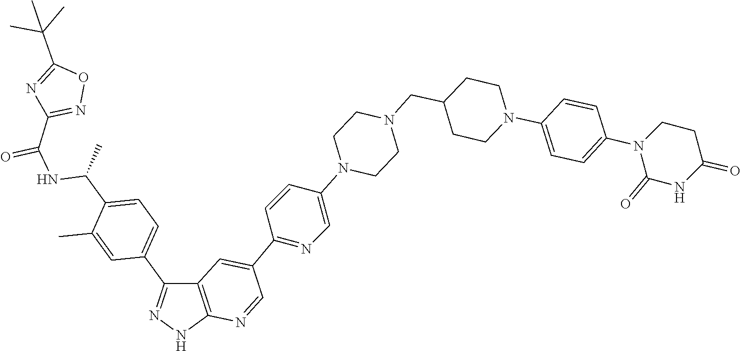 [SMILES: Cc1cc(-c2n[nH]c3ncc(-c4ccc(N5CCN(CC6CCN(c7ccc(N8CCC(=O)NC8=O)cc7)CC6)CC5)cn4)cc23)ccc1[C@@H](C)NC(=O)c1noc(C(C)(C)C)n1]

### Example 75: (R)-3-(tert-butyl)-N-(1-(4-(5-(5-(4-((1-(4-(2,4-dioxotetrahydropyrimidin-1(2H)-yl)phenyl)piperidin-4-yl)methyl)piperazin-1-yl)pyridin-2-yl)-1H-pyrazolo[3,4-b]pyridin-3-yl)-2-methylphenyl)ethyl)-1,2,4-oxadiazole-5-carboxamide

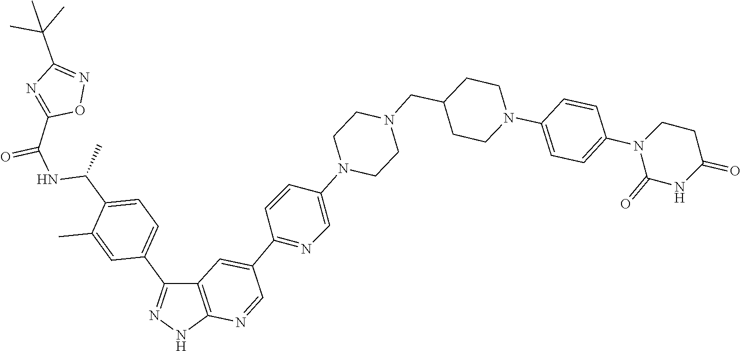 [SMILES: Cc1cc(-c2n[nH]c3ncc(-c4ccc(N5CCN(CC6CCN(c7ccc(N8CCC(=O)NC8=O)cc7)CC6)CC5)cn4)cc23)ccc1[C@@H](C)NC(=O)c1nc(C(C)(C)C)no1]

### Example 76: (R)-3-(tert-butyl)-N-(1-(4-(5-(6-(4-((1-(4-(2,4-dioxotetrahydropyrimidin-1(2H)-yl)phenyl)piperidin-4-yl)methyl)piperazin-1-yl)pyridin-3-yl)-1H-pyrazolo[3,4-b]pyridin-3-yl)-2-methylphenyl)ethyl)-1,2,4-oxadiazole-5-carboxamide

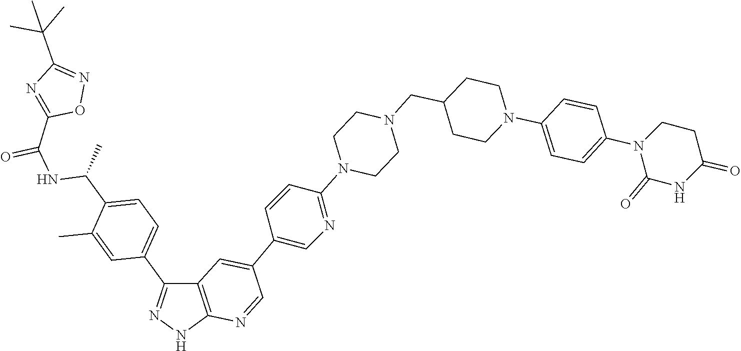 [SMILES: Cc1cc(-c2n[nH]c3ncc(-c4ccc(N5CCN(CC6CCN(c7ccc(N8CCC(=O)NC8=O)cc7)CC6)CC5)nc4)cc23)ccc1[C@@H](C)NC(=O)c1nc(C(C)(C)C)no1]

### Example 77: (R)-5-(tert-butyl)-N-(1-(4-(5-(6-(4-((1-(4-(2,4-dioxotetrahydropyrimidin-1(2H)-yl)-3-methylphenyl)piperidin-4-yl)methyl)piperazin-1-yl)pyridin-3-yl)-1H-pyrazolo[3,4-b]pyridin-3-yl)-2-methylphenyl)ethyl)-1,2,4-oxadiazole-3-carboxamide

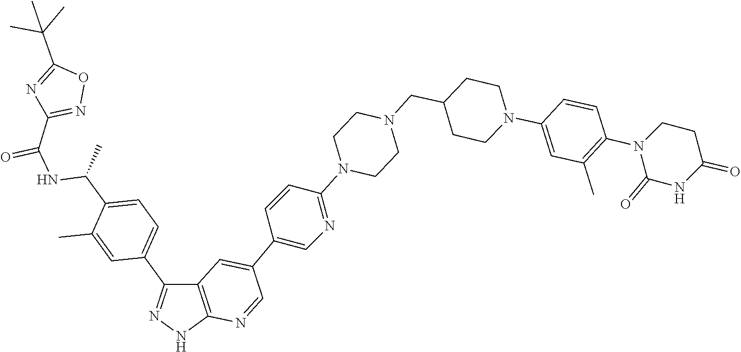 [SMILES: Cc1cc(-c2n[nH]c3ncc(-c4ccc(N5CCN(CC6CCN(c7ccc(N8CCC(=O)NC8=O)c(C)c7)CC6)CC5)nc4)cc23)ccc1[C@@H](C)NC(=O)c1noc(C(C)(C)C)n1]

### Example 78: (R)-5-(tert-butyl)-N-(1-(4-(5-(5-(4-((1-(4-(2,4-dioxotetrahydropyrimidin-1(2H)-yl)-3-methylphenyl)piperidin-4-yl)methyl)piperazin-1-yl)pyridin-2-yl)-1H-pyrazolo[3,4-b]pyridin-3-yl)-2-methylphenyl)ethyl)-1,2,4-oxadiazole-3-carboxamide

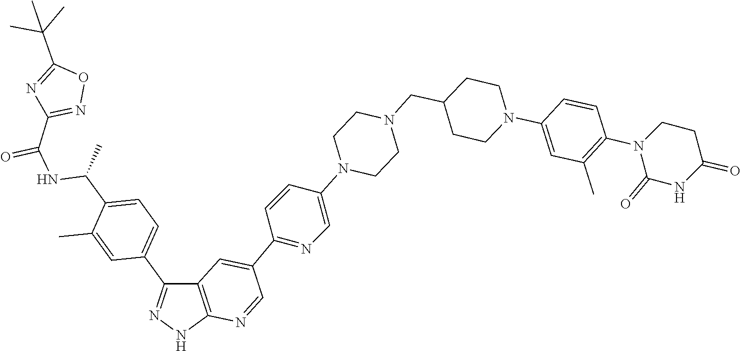 [SMILES: Cc1cc(-c2n[nH]c3ncc(-c4ccc(N5CCN(CC6CCN(c7ccc(N8CCC(=O)NC8=O)c(C)c7)CC6)CC5)cn4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.83 (s, 1H), 10.25 (s, 1H), 9.50 (d, J=8.0 Hz, 1H), 9.23 (s, 1H), 8.93 (s, 1H), 8.45 (s, 1H), 8.04 (d, J=8.0 Hz, 1H), 7.89 (d, J=8.0 Hz, 1H), 7.83 (s, 1H), 7.63 (d, J=8.0 Hz, 1H), 7.46 (d, J=8.0 Hz, 1H), 7.04 (d, J=8.3 Hz, 1H), 6.82-6.79 (m, 2H), 5.37 (s, 1H), 3.71-3.69 (m, 3H), 3.48 (s, 1H), 3.29 (s, 4H), 2.71-2.65 (m, 3H), 2.58-2.55 (m, 8H), 2.24-2.20 (m, 2H), 2.12 (s, 3H), 1.84-1.73 (m, 3H), 1.52 (d, J=8.0 Hz, 3H), 1.43 (s, 9H), 1.28-1.21 (m, 2H); [M+H]⁺=865.8.

### Example 79: (R)-5-(tert-butyl)-N-(1-(4-(5-(6-(4-((1-(4-(2,4-dioxotetrahydropyrimidin-1(2H)-yl)-3-fluorophenyl)piperidin-4-yl)methyl)piperazin-1-yl)pyridin-3-yl)-1H-pyrazolo[3,4-b]pyridin-3-yl)-2-methylphenyl)ethyl)-1,2,4-oxadiazole-3-carboxamide

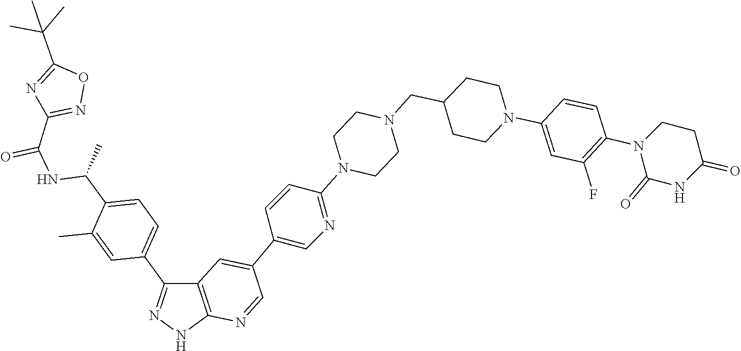 [SMILES: Cc1cc(-c2n[nH]c3ncc(-c4ccc(N5CCN(CC6CCN(c7ccc(N8CCC(=O)NC8=O)c(F)c7)CC6)CC5)nc4)cc23)ccc1[C@@H](C)NC(=O)c1noc(C(C)(C)C)n1]

### Example 80: (R)-5-(tert-butyl)-N-(1-(4-(5-(5-(4-((1-(4-(2,4-dioxotetrahydropyrimidin-1(2H)-yl)-3-fluorophenyl)piperidin-4-yl)methyl)piperazin-1-yl)pyridin-2-yl)-1H-pyrazolo[3,4-b]pyridin-3-yl)-2-methylphenyl)ethyl)-1,2,4-oxadiazole-3-carboxamide

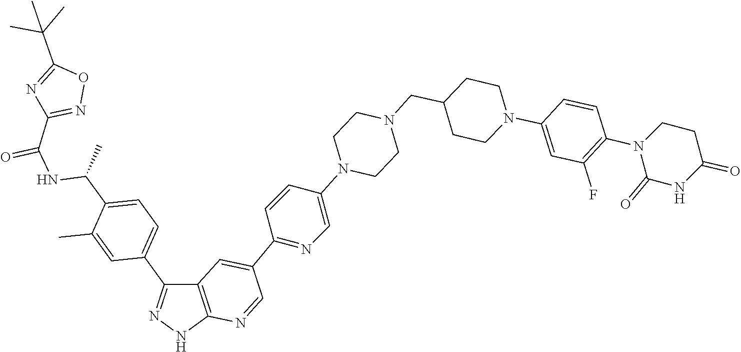 [SMILES: Cc1cc(-c2n[nH]c3ncc(-c4ccc(N5CCN(CC6CCN(c7ccc(N8CCC(=O)NC8=O)c(F)c7)CC6)CC5)cn4)cc23)ccc1[C@@H](C)NC(=O)c1noc(C(C)(C)C)n1]

### Example 86: 5-(tert-butyl)-N-((1R)-1-(4-(5-(4-(4-(3-(4-(2,6-dioxopiperidin-3-yl)-3-fluorophenoxy)propyl)piperazin-1-yl)phenyl)-1H-pyrazolo[3,4-b]pyridin-3-yl)-2-methylphenyl)ethyl)-1,2,4-oxadiazole-3-carboxamide

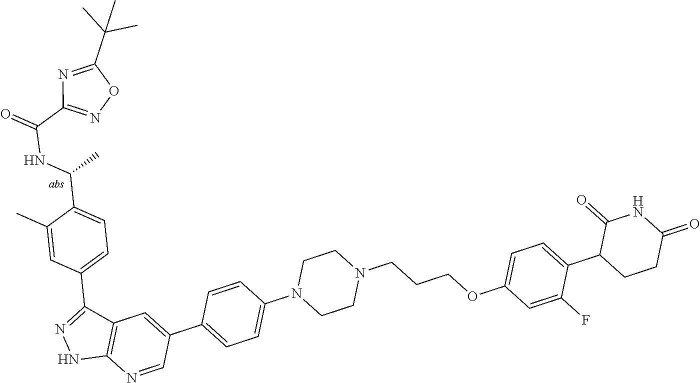 [SMILES: Cc1cc(-c2n[nH]c3ncc(-c4ccc(N5CCN(CCCOc6ccc(C7CCC(=O)NC7=O)c(F)c6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.77 (s, 1H), 10.85 (s, 1H), 9.49 (d, J=6.8 Hz, 1H), 8.82 (s, 1H), 8.58 (s, 1H), 7.91 (d, J=7.7 Hz, 1H), 7.85 (s, 1H), 7.69 (d, J=7.5 Hz, 2H), 7.60 (d, J=7.7 Hz, 1H), 7.20 (t, J=8.5 Hz, 1H), 7.07 (d, J=7.4 Hz, 2H), 6.83 (d, J=13.0 Hz, 1H), 6.77 (d, J=8.2 Hz, 1H), 5.41-5.32 (m, 1H), 4.06 (s, 2H), 3.96 (d, J=8.7 Hz, 1H), 3.22 (s, 4H), 3.10-2.54 (m, 11H), 2.24-2.11 (m, 1H), 2.03-1.89 (m, 3H), 1.51 (d, J=5.5 Hz, 3H), 1.42 (s, 9H); [M+H]⁺=828.8.

### Example 87: 3-(tert-butyl)-N-((1R)-1-(4-(5-(4-(4-(4-(2,6-dioxopiperidin-3-yl)-3-methylphenethyl)piperazin-1-yl)phenyl)-1H-pyrazolo[3,4-b]pyridin-3-yl)-2-methylphenyl)ethyl)-1,2,4-oxadiazole-5-carboxamide

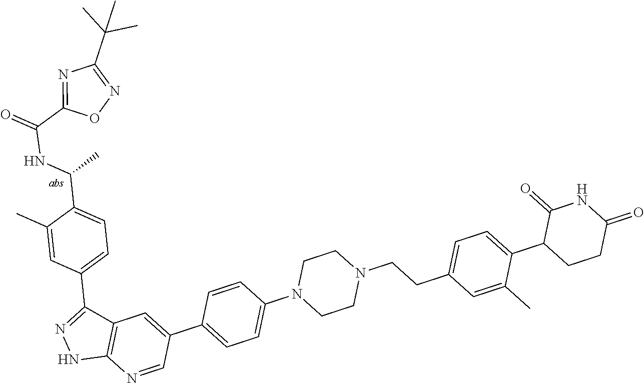 [SMILES: Cc1cc(CCN2CCN(c3ccc(-c4cnc5[nH]nc(-c6ccc([C@@H](C)NC(=O)c7nc(C(C)(C)C)no7)c(C)c6)c5c4)cc3)CC2)ccc1C1CCC(=O)NC1=O]

The titled compound was synthesized in the procedures similar to Example 1. ¹H NMR (400 MHz, DMSO) δ_{H} 13.78 (s, 1H), 10.83 (s, 1H), 9.91 (d, J=7.6 Hz, 1H), 8.82 (s, 1H), 8.59 (s, 1H), 7.93 (d, J=8.2 Hz, 1H), 7.86 (s, 1H), 7.69 (d, J=6.5 Hz, 2H), 7.62 (d, J=6.9 Hz, 1H), 7.15-6.96 (m, 6H), 5.41-5.31 (m, 1H), 4.00 (d, J=9.8 Hz, 2H), 3.23 (s, 4H), 2.81-2.53 (m, 10H), 2.35-2.09 (m, 4H), 2.02-1.92 (m, 2H), 1.54 (d, J=5.2 Hz, 3H), 1.37 (s, 9H); [M+H]⁺=794.8.

### Example 88 and 89: (S)-3-(tert-butyl)-N-(4-(5-(4-(2-(4-(4-(2,6-dioxopiperidin-3-yl)phenyl)piperidin-1-yl)ethyl)phenyl)-1H-pyrazolo[3,4-b]pyridin-3-yl)-2-methylbenzyl)-1,2,4-oxadiazole-5-carboxamide and (R)-3-(tert-butyl)-N-(4-(5-(4-(2-(4-(4-(2,6-dioxopiperidin-3-yl)phenyl)piperidin-1-yl)ethyl)phenyl)-1H-pyrazolo[3,4-b]pyridin-3-yl)-2-methylbenzyl)-1,2,4-oxadiazole-5-carboxamide

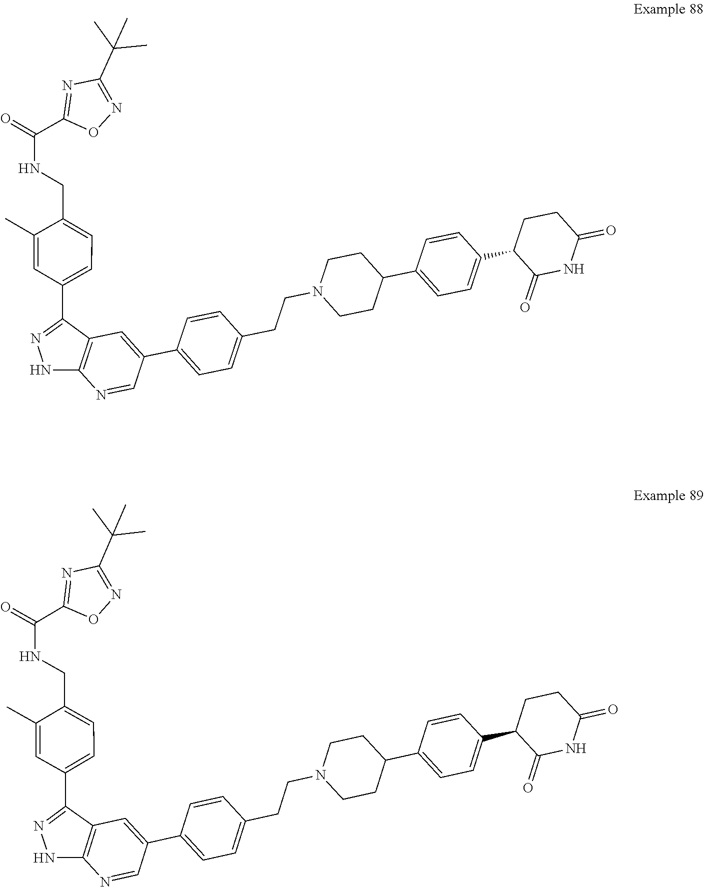 [SMILES: Cc1cc(-c2n[nH]c3ncc(-c4ccc(CCN5CCC(c6ccc([C@@H]7CCC(=O)NC7=O)cc6)CC5)cc4)cc23)ccc1CNC(=O)c1nc(C(C)(C)C)no1 ; Cc1cc(-c2n[nH]c3ncc(-c4ccc(CCN5CCC(c6ccc([C@H]7CCC(=O)NC7=O)cc6)CC5)cc4)cc23)ccc1CNC(=O)c1nc(C(C)(C)C)no1]

The Example 8 (130 mg) was separated by Prep-Chiral-HPLC with following conditions: Column: CHIRALPAK IF, 2*25 cm, 5 um; Mobile Phase A: Hexane (0.5% 2M NH₃-MeOH), Mobile Phase B: EtOH:DCM=1:1; Flow rate: 20 mL/min; Gradient: 70 B to 70 B in 17 min; Detector: 220/254 nm; RT1: 7.948 min; RT2: 11.336 min; Injection Volume: 1.5 ml; Number Of Runs: 3; This resulted in Example 89 (RT1: 7.948 min) (44.4 mg, 34.2%) and Example 88 (RT2: 11.336 min), (46.6 mg, 35.8%). Example 89: ¹H NMR (400 MHz, CDCl₃) δ 11.33 (s, 1H), 8.85 (d, J=2.1 Hz, 1H), 8.47 (d, J=2.1 Hz, 1H), 8.25 (s, 1H), 7.91-7.82 (m, 2H), 7.62-7.55 (m, 2H), 7.48 (d, J=7.9 Hz, 1H), 7.43-7.36 (m, 2H), 7.30-7.27 (m, 3H), 7.20-7.13 (m, 2H), 4.80-4.68 (m, 2H), 3.78 (dd, J=9.4, 5.3 Hz, 1H), 3.22 (d, J=10.9 Hz, 2H), 2.96 (dd, J=10.2, 6.0 Hz, 2H), 2.81-2.53 (m, 5H), 2.50 (s, 3H), 2.35-2.14 (m, 4H), 1.97-1.81 (m, 4H), 1.39 (s, 9H); [M+H]⁺=765.5. Example 88: ¹H NMR (400 MHz, CDCl₃) δ 11.63 (s, 1H), 8.86 (d, J=2.0 Hz, 1H), 8.47 (d, J=2.0 Hz, 1H), 8.39 (s, 1H), 7.91-7.82 (m, 2H), 7.61-7.54 (m, 2H), 7.48 (d, J=7.9 Hz, 1H), 7.42-7.36 (m, 2H), 7.31-7.27 (m, 3H), 7.19-7.13 (m, 2H), 4.78-4.69 (m, 2H), 3.78 (dd, J=9.3, 5.3 Hz, 1H), 3.21 (d, J=11.1 Hz, 2H), 2.96 (dd, J=10.2, 6.0 Hz, 2H), 2.79-2.52 (m, 5H), 2.50 (s, 3H), 2.35-2.14 (m, 4H), 1.97-1.81 (m, 4H), 1.39 (s, 9H); [M+H]⁺=765.5.

### Example 90: (R)-5-(tert-butyl)-N-(1-(4-(5-(4-(4-((1-(2-(2,4-dioxotetrahydropyrimidin-1(2H)-yl)-5-fluorophenyl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

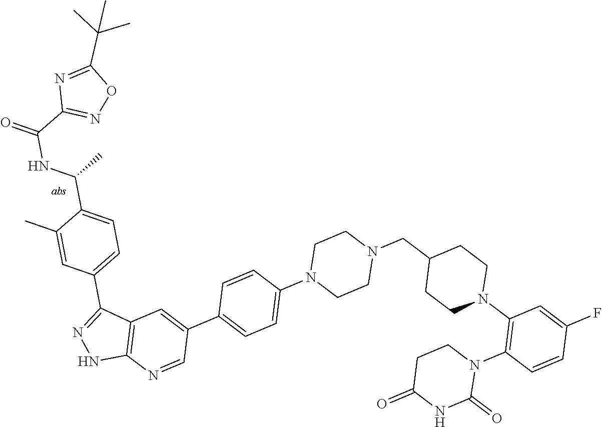

The titled compound was synthesized in the procedures similar to Example 1. ¹H NMR (400 MHz, DMSO) δ_{H} 13.77 (s, 1H), 10.38 (s, 1H), 9.49 (d, J=6.5 Hz, 1H), 8.82 (s, 1H), 8.58 (s, 1H), 7.91 (d, J=8.1 Hz, 1H), 7.85 (s, 1H), 7.69 (d, J=7.0 Hz, 2H), 7.61 (s, 1H), 7.24 (s, 1H), 7.06 (d, J=8.4 Hz, 2H), 6.94 (d, J=11.0 Hz, 1H), 6.87 (s, 1H), 5.36 (s, 1H), 3.72 (s, 1H), 3.48 (s, 1H), 3.26-3.00 (m, 7H), 2.81 (s, 1H), 2.74-2.53 (m, 9H), 2.26 (s, 2H), 2.05-1.61 (m, 5H), 1.51 (s, 3H), 1.42 (s, 9H); [M+H]⁺=869.0.

### Example 91: (R)-5-(tert-butyl)-N-(4-(5-(4-(4-(2-(1-(4-(2,6-dioxopiperidin-3-yl)phenyl)piperidin-4-yl)ethyl)piperazin-1-yl)phenyl)-1H-pyrazolo[3,4-b]pyridin-3-yl)-2-methylbenzyl)-1,2,4-oxadiazole-3-carboxamide

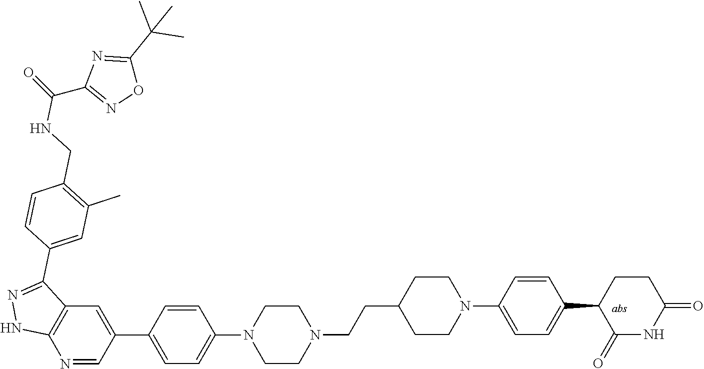 [SMILES: Cc1cc(-c2n[nH]c3ncc(-c4ccc(N5CCN(CCC6CCN(c7ccc([C@H]8CCC(=O)NC8=O)cc7)CC6)CC5)cc4)cc23)ccc1CNC(=O)c1noc(C(C)(C)C)n1]

The titled compound was synthesized in the procedures similar to Example 1. ¹H NMR (400 MHz, DMSO) δ_{H} 13.78 (s, 1H), 10.78 (s, 1H), 9.47 (s, 1H), 8.82 (s, 1H), 8.58 (s, 1H), 7.95-7.83 (m, 2H), 7.69 (d, J=8.0 Hz, 2H), 7.40 (d, J=4.0 Hz, 1H), 7.05 (dd, J=12.0, 8.0 Hz, 4H), 6.89 (d, J=8.0 Hz, 2H), 4.52 (s, 2H), 3.75-3.60 (m, 3H), 3.32-3.29 (m, 1H), 3.20 (s, 4H), 2.71-2.56 (m, 5H), 2.47-2.33 (m, 6H), 2.19-2.07 (m, 1H), 2.06-1.96 (m, 1H), 1.77 (d, J=12.0 Hz, 2H), 1.56-1.41 (m, 12H), 1.34-1.20 (m, 3H); [M+H]⁺=849.9.

### Example 92: (S)-5-(tert-butyl)-N-(4-(5-(4-(4-(2-(1-(4-(2,6-dioxopiperidin-3-yl)phenyl)piperidin-4-yl)ethyl)piperazin-1-yl)phenyl)-1H-pyrazolo[3,4-b]pyridin-3-yl)-2-methylbenzyl)-1,2,4-oxadiazole-3-carboxamide

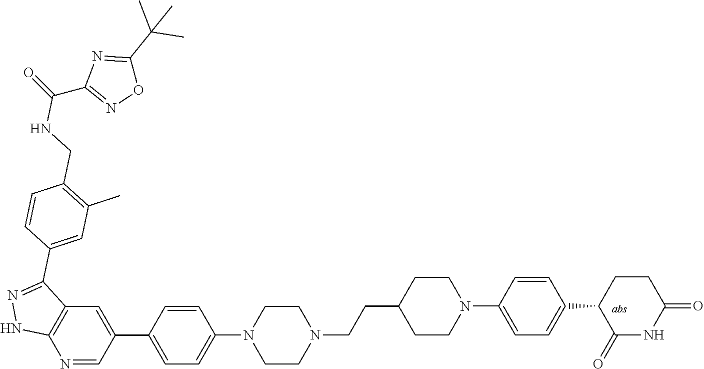 [SMILES: Cc1cc(-c2n[nH]c3ncc(-c4ccc(N5CCN(CCC6CCN(c7ccc([C@@H]8CCC(=O)NC8=O)cc7)CC6)CC5)cc4)cc23)ccc1CNC(=O)c1noc(C(C)(C)C)n1]

The titled compound was synthesized in the procedures similar to Example 1. ¹H NMR (400 MHz, DMSO) δ_{H} 13.78 (s, 1H), 10.78 (s, 1H), 9.47 (s, 1H), 8.82 (s, 1H), 8.58 (s, 1H), 7.95-7.83 (m, 2H), 7.69 (d, J=8.0 Hz, 2H), 7.40 (d, J=4.0 Hz, 1H), 7.05 (dd, J=12.0, 8.0 Hz, 4H), 6.89 (d, J=8.0 Hz, 2H), 4.52 (s, 2H), 3.75-3.60 (m, 3H), 3.32-3.29 (m, 1H), 3.20 (s, 4H), 2.71-2.56 (m, 5H), 2.47-2.33 (m, 6H), 2.19-2.07 (m, 1H), 2.06-1.96 (m, 1H), 1.77 (d, J=12.0 Hz, 2H), 1.56-1.41 (m, 12H), 1.34-1.20 (m, 3H); [M+H]⁺=849.8.

### Example 93: 2-(tert-butyl)-N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylbenzyl)thiazole-4-carboxamide

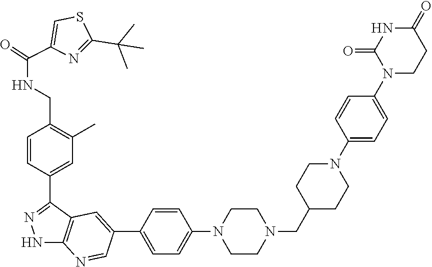 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1csc(C(C)(C)C)n1]

The titled compound was synthesized in the procedures similar to Example 1. ¹H NMR (400 MHz, DMSO) δ_{H} 13.76 (s, 1H), 10.26 (s, 1H), 8.82 (s, 1H), 8.72 (s, 1H), 8.58 (s, 1H), 8.18 (s, 1H), 7.91-7.87 (m, 2H), 7.68 (d, J=7.6 Hz, 2H), 7.38 (d, J=8.0 Hz, 1H), 7.13 (d, J=7.2 Hz, 2H), 7.06 (d, J=7.6 Hz, 2H), 6.93 (d, J=8.4 Hz, 2H), 4.54 (s, 2H), 3.69 (brs, 4H), 3.21 (brs, 4H), 2.69-2.50 (m, 7H), 2.45 (s, 3H), 2.29 (brs, 2H), 1.87-1.64 (m, 3H), 1.43 (s, 9H), 1.23 (brs, 3H); [M+H]⁺=851.8.

### Example 94: 2-(tert-butyl)-N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylbenzyl)thiazole-5-carboxamide

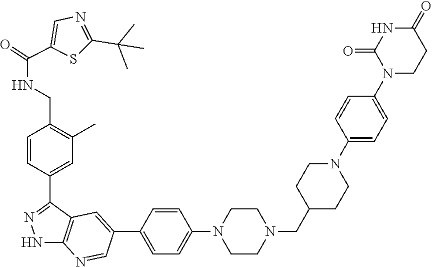 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1CNC(=O)c1cnc(C(C)(C)C)s1]

The titled compound was synthesized in the procedures similar to Example 1. ¹H NMR (400 MHz, DMSO) δ_{H} 13.78 (s, 1H), 10.27 (s, 1H), 9.11 (s, 1H), 8.82 (s, 1H), 8.58 (s, 1H), 8.35 (s, 1H), 7.91 (d, J=12.4 Hz, 2H), 7.69 (d, J=7.2 Hz, 2H), 7.40 (d, J=7.6 Hz, 1H), 7.13 (d, J=7.6 Hz, 2H), 7.06 (d, J=6.8 Hz, 2H), 6.93 (d, J=7.6 Hz, 2H), 4.51 (s, 2H), 3.69 (brs, 4H), 3.21 (brs, 4H), 2.68-2.50 (m, 7H), 2.44 (s, 3H), 2.24 (brs, 2H), 1.88-1.65 (m, 3H), 1.40 (s, 9H), 1.24 (brs, 3H); [M+H]⁺=851.8.

### Example 95: (R)-5-(tert-butyl)-N-(1-(4-(5-(3-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

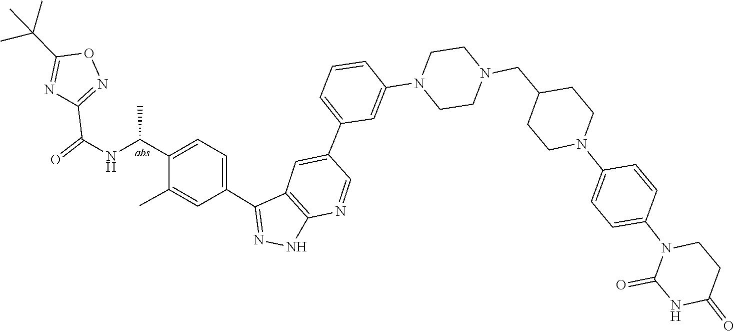 [SMILES: Cc1cc(-c2n[nH]c3ncc(-c4cccc(N5CCN(CC6CCN(c7ccc(N8CCC(=O)NC8=O)cc7)CC6)CC5)c4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.84 (s, 1H), 10.27 (s, 1H), 9.49 (d, J=7.3 Hz, 1H), 8.84 (s, 1H), 8.65 (s, 1H), 7.93 (d, J=8.2 Hz, 1H), 7.86 (s, 1H), 7.61 (d, J=7.6 Hz, 1H), 7.40-7.26 (m, 2H), 7.20 (s, 1H), 7.13 (d, J=8.0 Hz, 2H), 6.99 (d, J=7.1 Hz, 1H), 6.93 (d, J=7.7 Hz, 3H), 5.38-5.34 (m, 2H), 3.70-3.66 (m, 4H), 3.28-3.24 (m, 4H), 2.70-2.65 (m, 7H), 2.52 (s, 4H), 2.25-2.22 (m, 3H), 1.91 (s, 1H), 1.84-1.80 (m, 2H), 1.51 (d, J=6.5 Hz, 3H), 1.42 (s, 9H); [M+H]⁺=850.8.

### Example 96: (R)—N-(1-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylphenyl)ethyl)-2-fluoro-4-(2-hydroxypropan-2-yl)benzamide

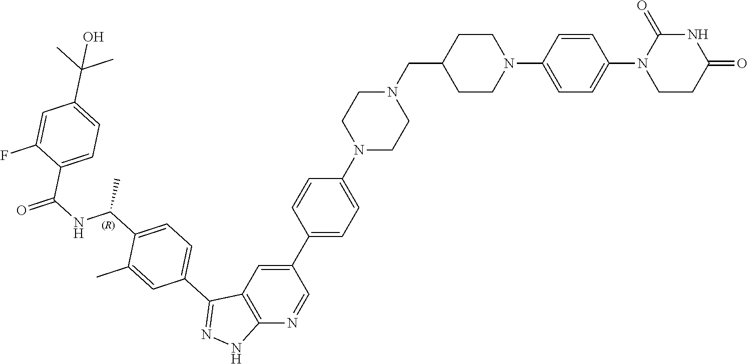

The titled compound was synthesized in the procedures similar to Example 1. ¹H NMR (400 MHz, DMSO) δ_{H} 13.77 (s, 1H), 10.27 (s, 1H), 8.82 (s, 2H), 8.59 (s, 1H), 7.98-7.83 (m, 2H), 7.81-7.64 (m, 2H), 7.58 (d, J=8.0 Hz, 1H), 7.54-7.46 (m, 1H), 7.38-7.27 (m, 2H), 7.21-7.02 (m, 4H), 7.00-6.87 (m, 2H), 5.40-5.20 (m, 2H), 3.77-3.62 (m, 5H), 3.25-3.13 (m, 5H), 2.72-2.64 (m, 5H), 2.52 (s, 4H), 2.29-2.16 (m, 1H), 1.95-1.88 (m, 2H), 1.88-1.79 (m, 2H), 1.50-1.40 (m, 9H), 1.29-1.18 (m, 2H); [M+H]⁺=878.6.

### Example 97: (R)-5-(tert-butyl)-N-(1-(4-(5-(4-(4-((1-(4-(2,4-dioxotetrahydropyrimidin-1(2H)-yl)phenyl)piperidin-4-yl)methyl)piperazin-1-yl)-3-fluorophenyl)-1H-pyrazolo[3,4-b]pyridin-3-yl)-2-methylphenyl)ethyl)-1,2,4-oxadiazole-3-carboxamide

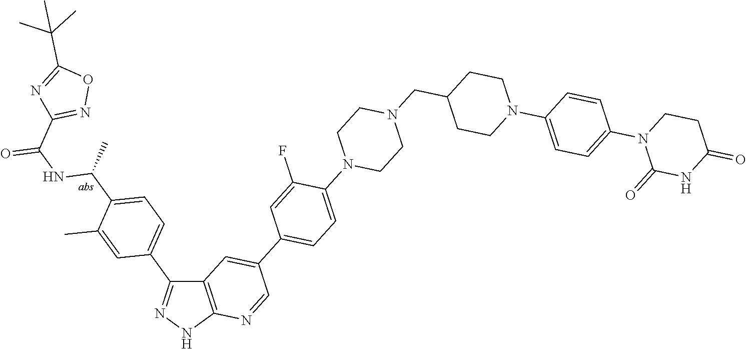 [SMILES: Cc1cc(-c2n[nH]c3ncc(-c4ccc(N5CCN(CC6CCN(c7ccc(N8CCC(=O)NC8=O)cc7)CC6)CC5)c(F)c4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.84 (s, 1H), 10.27 (s, 1H), 9.50 (s, 1H), 8.86 (s, 1H), 8.67 (s, 1H), 7.97-7.90 (m, 1H), 7.86 (s, 1H), 7.75-7.62 (m, 1H), 7.62-7.58 (m, 1H), 7.18-7.10 (m, 2H), 7.00-6.90 (m, 2H), 5.42-5.30 (m, 1H), 3.80-3.60 (m, 5H), 3.30-3.20 (m, 2H), 3.30-3.10 (m, 3H), 2.68 (s, 3H), 2.60-2.52 (m, 2H), 2.35-2.20 (m, 2H), 1.95-1.80 (m, 3H), 1.79-1.67 (m, 1H), 1.55-1.16 (m, 3H), 1.42 (s, 9H), 1.30-1.15 (m, 2H); [M+H]⁺=868.9.

### Example 98: (R)-3-(tert-butyl)-N-(1-(4-(5-(4-(4-((1-(4-(2,4-dioxotetrahydropyrimidin-1(2H)-yl)-3-methylphenyl)piperidin-4-yl)methyl)piperazin-1-yl)phenyl)-1H-pyrazolo[3,4-b]pyridin-3-yl)-2-methylphenyl)ethyl)-1,2,4-oxadiazole-5-carboxamide

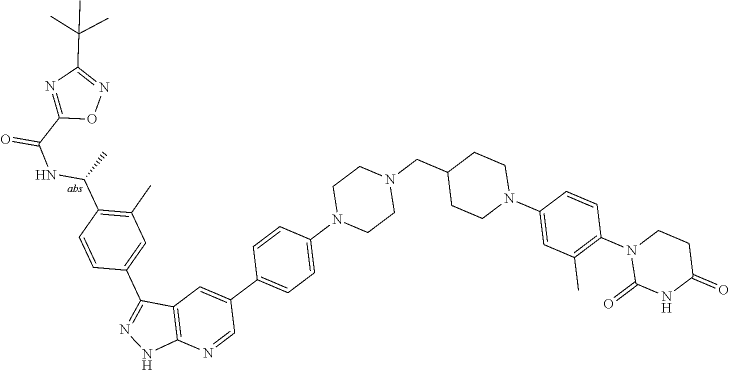 [SMILES: Cc1cc(-c2n[nH]c3ncc(-c4ccc(N5CCN(CC6CCN(c7ccc(N8CCC(=O)NC8=O)c(C)c7)CC6)CC5)cc4)cc23)ccc1[C@@H](C)NC(=O)c1nc(C(C)(C)C)no1]

A mixture of (R)-3-(tert-butyl)-N-(1-(2-methyl-4-(5-(4-(piperazin-1-yl)phenyl)-1H-pyrazolo[3,4-b]pyridin-3-yl)phenyl)ethyl)-1,2,4-oxadiazole-5-carboxamide (80 mg, 0.142 mmol), 1-(4-(2,4-dioxotetrahydropyrimidin-1(2H)-yl)-3-methylphenyl)piperidine-4-carbaldehyde (66.8 mg, 0.213 mmol) and NaOAc (34.9 mg, 0.426 mmol) in DCM (4 mL): MeOH (4 mL), was stirred in a round bottom flask for 5 mins. Then HOAc was added dropwise (0.06 mL). The mixture was stirred at room temperature overnight. Then NaBH(OAc)₃ (150.5 mg, 0.71 mmol) was added. The mixture was stirred at room temperature for 1.5 h. After the reaction was completed determined by LCMS, the reaction mixture was concentrated in vacuo. The residue was purified by silica gel column chromatography (DCM:MeOH=95%: 5%) to give the product (53.72 mg, 43%). ¹H NMR (400 MHz, DMSO) δ_{H} 13.78 (s, 1H), 10.25 (s, 1H), 9.91 (d, J=8.0 Hz, 1H), 8.82 (s, 1H), 8.59 (s, 1H), 7.92 (d, J=8.0 Hz, 1H), 7.86 (d, J=8.0 Hz, 2H), 7.68 (d, J=8.0 Hz, 1H), 7.63 (s, 1H), 7.06 (s, 4H), 6.81 (d, J=11.3 Hz, 3H), 5.35 (s, 1H), 3.75-3.67 (m, 3H), 3.48 (s, 1H), 3.22 (s, 4H), 3.07-2.82 (m, 2H), 2.73-2.64 (m, 4H), 2.53 (s, 2H), 2.23 (s, 2H), 2.12 (s, 3H), 1.82 (d, J=8.0 Hz, 2H), 1.76-1.67 (m, 1H), 1.53 (d, J=8.0 Hz, 3H), 1.36 (s, 12H), 1.28-1.15 (m, 3H); [M+H]⁺=864.6.

### Example 99: 5-(tert-butyl)-N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-3-fluoro-2-methylbenzyl)-1,2,4-oxadiazole-3-carboxamide

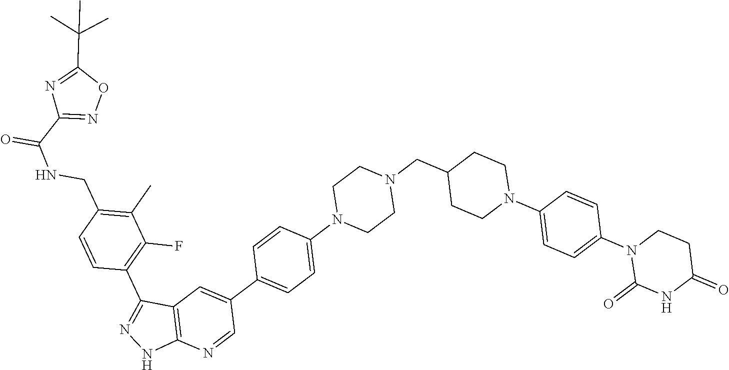 [SMILES: Cc1c(CNC(=O)c2noc(C(C)(C)C)n2)ccc(-c2n[nH]c3ncc(-c4ccc(N5CCN(CC6CCN(c7ccc(N8CCC(=O)NC8=O)cc7)CC6)CC5)cc4)cc23)c1F]

The titled compound was synthesized in the procedures similar to Example 1. ¹H NMR (400 MHz, DMSO) δ_{H} 13.96 (s, 1H), 10.27 (s, 1H), 9.53 (s, 1H), 8.84 (s, 1H), 8.30 (s, 1H), 7.71-7.59 (m, 3H), 7.27 (d, J=8.0 Hz, 1H), 7.14 (d, J=8.0 Hz, 2H), 7.14 (d, J=8.0 Hz, 2H), 6.95 (d, J=8.0 Hz, 2H), 4.56 (s, 2H), 3.69 (s, 4H), 3.21 (s, 4H), 2.72-2.66 (m, 4H), 2.36 (s, 3H), 2.23 (s, 2H), 1.81 (d, J=8.0 Hz, 1H), 1.76-1.66 (m, 1H), 1.44 (s, 9H), 1.29-1.23 (m, 5H); [M+H]⁺=854.7.

### Example 100: 5-(tert-butyl)-N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-5-fluoro-2-methylbenzyl)-1,2,4-oxadiazole-3-carboxamide

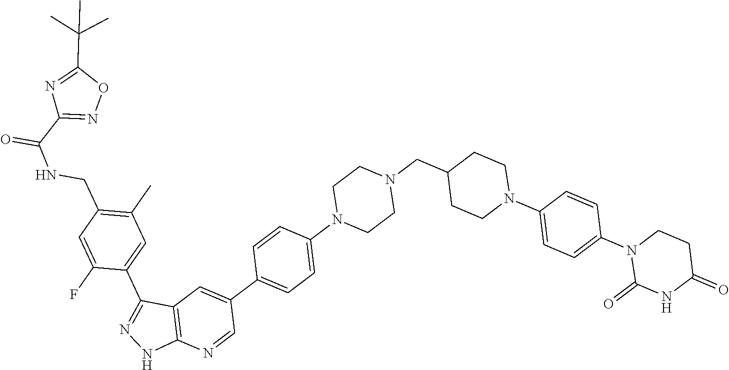 [SMILES: Cc1cc(-c2n[nH]c3ncc(-c4ccc(N5CCN(CC6CCN(c7ccc(N8CCC(=O)NC8=O)cc7)CC6)CC5)cc4)cc23)c(F)cc1CNC(=O)c1noc(C(C)(C)C)n1]

The titled compound was synthesized in the procedures similar to Example 1. ¹H NMR (400 MHz, DMSO) δ_{H} 14.00 (s, 1H), 10.27 (s, 2H), 9.54 (s, 1H), 8.84 (s, 1H), 8.28 (s, 2H), 7.70 (d, J=8.0 Hz, 1H), 7.61 (d, J=8.0 Hz, 2H), 7.24 (d, J=12.0 Hz, 1H), 7.12 (d, J=8.0 Hz, 2H), 7.04 (d, J=8.0 Hz, 2H), 6.94 (d, J=8.0 Hz, 2H), 4.51 (s, 2H), 3.69 (s, 4H), 3.21 (s, 4H), 2.72-2.66 (m, 4H), 2.40 (s, 4H), 2.23 (s, 2H), 1.81 (d, J=8.0 Hz, 2H), 1.76-1.70 (m, 1H), 1.44 (s, 9H), 1.30-1.21 (m, 5H); [M+H]⁺₌854.8.

### Example 101: 5-(tert-butyl)-N-((1R)-1-(4-(5-(4-(4-(2-(5-(2,6-dioxopiperidin-3-yl)pyridin-2-yl)ethyl)piperazin-1-yl)phenyl)-1H-pyrazolo[3,4-b]pyridin-3-yl)-2-methylphenyl)ethyl)-1,2,4-oxadiazole-3-carboxamide

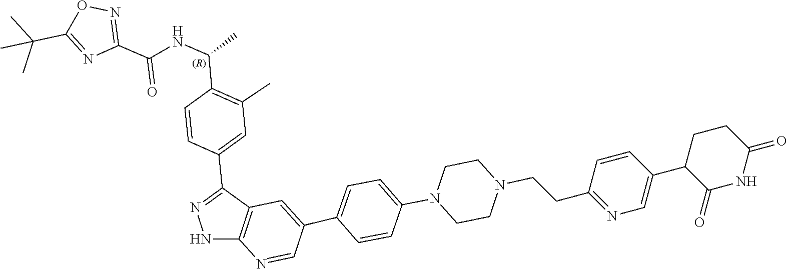

The titled compound was synthesized in the procedures similar to Example 1. ¹H NMR (400 MHz, DMSO) δ_{H} 13.77 (s, 1H), 10.91 (s, 1H), 9.49 (d, J=8.0 Hz, 1H), 8.82 (s, 1H), 8.58 (s, 1H), 8.36 (s, 1H), 7.92 (d, J=12.0 Hz, 1H), 7.85 (s, 1H), 7.69 (d, J=8.0 Hz, 2H), 7.59 (t, J=8.0 Hz, 2H), 7.30 (d, J=8.0 Hz, 1H), 7.07 (d, J=8.0 Hz, 2H), 5.43-5.30 (m, 1H), 3.96-3.86 (m, 1H), 3.27-3.16 (m, 4H), 2.99-2.89 (m, 2H), 2.77-2.56 (m, 8H), 2.46 (s, 3H), 2.33-2.22 (m, 1H), 2.06-1.98 (m, 1H), 1.51 (d, J=8.0 Hz, 3H), 1.42 (s, 9H); [M+H]⁺=781.4.

### Example 102: (R)-5-(tert-butyl)-N-(1-(4-(5-(4-(4-((1-(4-(2,4-dioxotetrahydropyrimidin-1(2H)-yl)phenyl)piperidin-4-yl)methyl)piperazin-1-yl)-3-methylphenyl)-1H-pyrazolo[3,4-b]pyridin-3-yl)-2-methylphenyl)ethyl)-1,2,4-oxadiazole-3-carboxamide

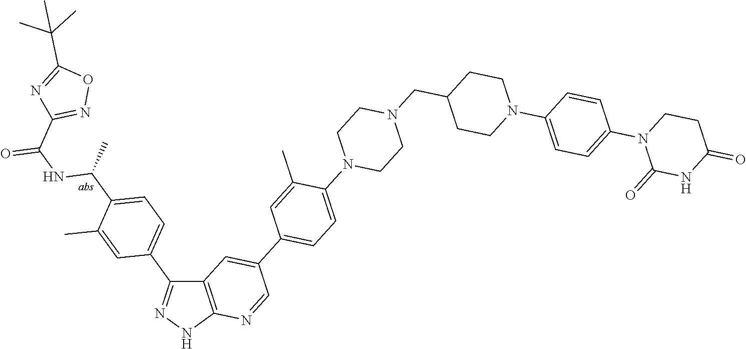 [SMILES: Cc1cc(-c2n[nH]c3ncc(-c4ccc(N5CCN(CC6CCN(c7ccc(N8CCC(=O)NC8=O)cc7)CC6)CC5)c(C)c4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.82 (s, 1H), 10.27 (s, 1H), 9.55-9.45 (m, 1H), 8.82 (s, 1H), 8.61 (s, 1H), 7.91 (d, J=8.0 Hz, 1H), 7.85 (s, 1H), 7.70-7.55 (m, 3H), 7.20-7.10 (m, 3H), 7.00-6.90 (m, 2H), 5.40-5.30 (m, 1H), 3.75-3.65 (m, 5H), 3.30-3.10 (m, 2H), 2.95-2.85 (m, 3H), 2.72-2.62 (m, 5H), 2.60-2.55 (m, 2H), 2.40-2.30 (m, 3H), 2.30-2.20 (m, 1H), 1.90-1.80 (m, 2H), 1.78-1.65 (m, 1H), 1.51 (d, J=5.6 Hz, 3H), 1.42 (s, 9H), 1.30-1.20 (m, 4H); [M+H]⁺=864.9.

### Example 103: 5-(tert-butyl)-N-(4-(5-(5-(4-(4-(2,6-dioxopiperidin-3-yl)phenethyl)piperazin-1-yl)pyridin-2-yl)-1H-pyrazolo[3,4-b]pyridin-3-yl)-2-methylbenzyl)-1,2,4-oxadiazole-3-carboxamide

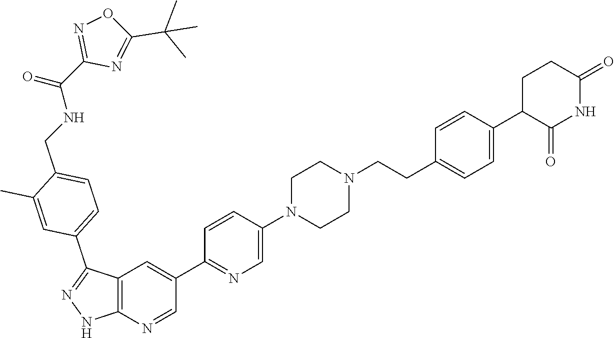 [SMILES: Cc1cc(-c2n[nH]c3ncc(-c4ccc(N5CCN(CCc6ccc(C7CCC(=O)NC7=O)cc6)CC5)cn4)cc23)ccc1CNC(=O)c1noc(C(C)(C)C)n1]

### Step 1: N-(4-(5-bromo-1-(tetrahydro-2H-pyran-2-yl)-1H-pyrazolo[3,4-b]pyridin-3-yl)-2-methylbenzyl)-5-(tert-butyl)-1,2,4-oxadiazole-3-carboxamide

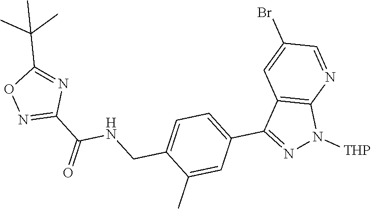

To a solution of 5-(tert-butyl)-N-(2-methyl-4-(4,4,5,5-tetramethyl-1,3,2-dioxaborolan-2-yl)benzyl)-1,2,4-oxadiazole-3-carboxamide (12 g, 30 mmol) in 1,4-dioxane (100 mL) and H₂O (10 mL) were added 5-bromo-3-iodo-1-(tetrahydro-2H-pyran-2-yl)-1H-indazole (12.2 g, 30 mmol), Pd(dppf)Cl₂ (1.5 g, 3 mmol) and K₂CO₃ (8.3 g, 60 mmol). The mixture was stirred overnight at 85° C. under N₂. The solvent was removed under reduced pressure and the crude product was purified with silica gel column chromatography (PE:EtOAc=10:1˜4:1 gradient elution) to give the product (13 g, 78%). [M+H]⁺=553.2.

### Step 2: 5-(tert-butyl)-N-(2-methyl-4-(1-(tetrahydro-2H-pyran-2-yl)-5-(4,4,5,5-tetramethyl-1,3,2-dioxaborolan-2-yl)-1H-pyrazolo[3,4-b]pyridin-3-yl)benzyl)-1,24-oxadiazole-3-carboxamide

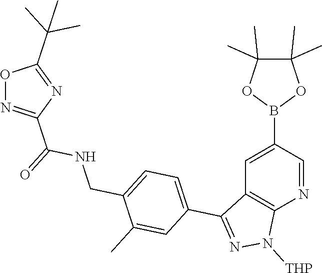

To a solution of N-(4-(5-bromo-1-(tetrahydro-2H-pyran-2-yl)-1H-pyrazolo[3,4-b]pyridin-3-yl)-2-methylbenzyl)-5-(tert-butyl)-1,2,4-oxadiazole-3-carboxamide (6.2 g, 11.2 mmol) in 1,4-dioxane (100 mL) were added 4,4,4′,4′,5,5,5′,5′-octamethyl-2,2′-bi(1,3,2-dioxaborolane) (5.7 g, 22.4 mmol), Pd(dppf)Cl₂ (1.2 g, 1.7 mmol) and KOAc (2.2 g, 22.4 mmol). The mixture was stirred overnight at 100° C. under N₂. The solvent was removed under reduced pressure and the crude product was purified with silica gel column chromatography (PE:EtOAc=10:1˜2:1 gradient elution) to give the product (1.0 g, 14.9%). [M+H]⁺=601.3.

### Step 3: tert-butyl 4-(6-(3-(4-((5-(tert-butyl)-1,2,4-oxadiazole-3-carboxamido)methyl)-3-methylphenyl)-1-(tetrahydro-2H-pyran-2-yl)-1H-pyrazolo[3,4-b]pyridin-5-yl)pyridin-3-yl)piperazine-1-carboxylate

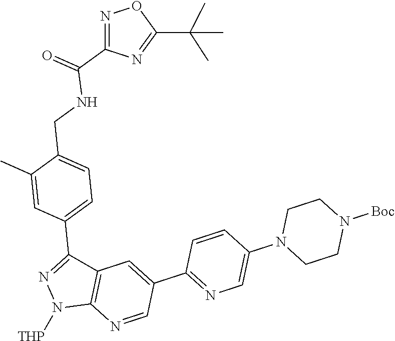

To a solution of tert-butyl 4-(6-bromopyridin-3-yl)piperazine-1-carboxylate (435 mg, 1.414 mmol) in 1,4-dioxane (100 mL) and H₂O (30 mL) were added X-Phos (101 mg, 0.212 mmol), RuPhos-Pd-G3 (169 mg, 0.212 mmol) and K₃PO₄ (600 mg, 2.828 mmol). A solution of 5-(tert-butyl)-N-(2-methyl-4-(1-(tetrahydro-2H-pyran-2-yl)-5-(4,4,5,5-tetramethyl-1,3,2-dioxaborolan-2-yl)-1H-pyrazolo[3,4-b]pyridin-3-yl)benzyl)-1,2,4-oxadiazole-3-carboxamide (850 mg, 1.414 mmol) in 1,4-dioxane (100 mL) was added dropwise at 90° C. under N₂. The mixture was stirred overnight at 100° C. under N₂. The solvent was removed under reduced pressure and the crude product was purified with silica gel column chromatography (PE:EtOAc=1:1˜0:1 gradient elution) to give the product (280 mg, 29%). [M+H]⁺=736.4.

### Step 4: 5-(tert-butyl)-N-(2-methyl-4-(5-(5-(piperazin-1-yl)pyridin-2-yl)-1H-pyrazolo[3,4-b]pyridin-3-yl)benzyl)-1,2,4-oxadiazole-3-carboxamide

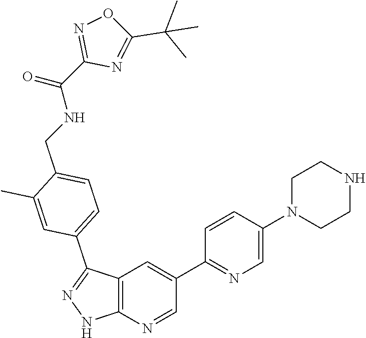

To a solution of tert-butyl 4-(6-(3-(4-((5-(tert-butyl)-1,2,4-oxadiazole-3-carboxamido)methyl)-3-methylphenyl)-1-(tetrahydro-2H-pyran-2-yl)-1H-pyrazolo[3,4-b]pyridin-5-yl)pyridin-3-yl)piperazine-1-carboxylate (280 mg, 0.38 mmol) in DCM (10 mL) was added TFA (10 mL). The mixture was stirred overnight at room temperature. The solvent was removed under vacuo to give the crude product which was further purified by pre-HPLC to give the product (110 mg, 52.3%). [M+H]⁺=552.3.

### Step 5: 5-(tert-butyl)-N-(4-(5-(5-(4-(4-(2,6-dioxopiperidin-3-yl)phenethyl)piperazin-1-yl)pyridin-2-yl)-1H-pyrazolo[3,4-b]pyridin-3-yl)-2-methylbenzyl)-1,2,4-oxadiazole-3-carboxamide

To a solution of 5-(tert-butyl)-N-(2-methyl-4-(5-(5-(piperazin-1-yl)pyridin-2-yl)-1H-pyrazolo[3,4-b]pyridin-3-yl)benzyl)-1,2,4-oxadiazole-3-carboxamide (60 mg, 0.11 mmol) in DCM (20 mL) and MeOH (5 mL) were added 2-(4-(2,6-dioxopiperidin-3-yl)phenyl)acetaldehyde (30 mg, 0.132 mmol) and AcOH (0.04 mL). The mixture was stirred overnight at room temperature under N₂. To the mixture was added NaBH(OAc)₃ (47 mg, 0.22 mmol), then stirred for another 3 h at room temperature. The reaction was quenched by NaHCO₃ (aq, 20 mL) and then extracted with DCM (30 mL×3). The organic layer was dried over with Na₂SO₄, filtered and concentrated to give the crude product which was purified by pre-TLC to give the product (32 mg, 38%). ¹H NMR (400 MHz, DMSO) δ_{H} 13.83 (s, 1H), 10.83 (s, 1H), 9.47 (s, 1H), 9.23 (s, 1H), 8.94 (s, 1H), 8.45 (s, 1H), 8.02 (d, J=8.0 Hz, 2H), 7.88-7.87 (m, 2H), 7.47-7.42 (m, 2H), 7.23-7.15 (m, 4H), 4.53 (s, 2H), 3.82 (d, J=8.0 Hz, 1H), 3.32-3.30 (m, 4H), 2.63-2.61 (m, 3H), 2.56-2.51 (m, 7H), 2.46 (s, 3H), 2.19-2.16 (m, 1H), 2.04 (s, 1H), 1.44 (s, 9H); [M+H]⁺=767.4.

### Example 104: (R)—N-(1-(4-(5-(4-(4-(2-(1-(3-chloro-4-(2,4-dioxotetrahydropyrimidin-1(2H)-yl)phenyl)piperidin-4-yl)ethyl)piperazin-1-yl)phenyl)-1H-pyrazolo[3,4-b]pyridin-3-yl)-2-methylphenyl)ethyl)-2-fluoro-4-(2-hydroxypropan-2-yl)benzamide

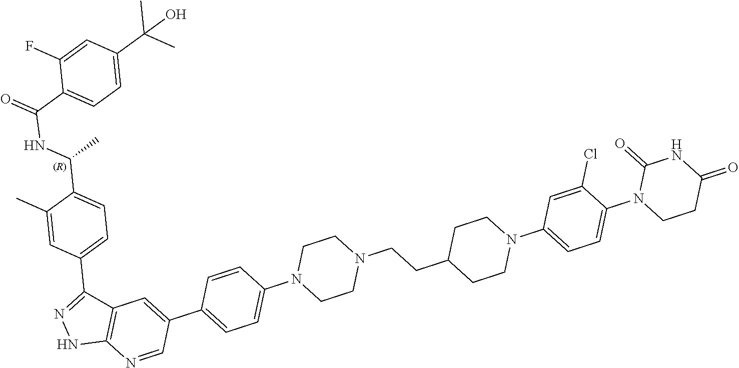

The titled compound was synthesized in the procedures similar to Example 1. ¹H NMR (400 MHz, DMSO) δ_{H} 13.78 (s, 1H), 10.37 (s, 1H), 8.82 (s, 2H), 8.58 (s, 1H), 8.39-8.32 (m, 2H), 7.93-7.82 (m, 2H), 7.75-7.64 (m, 2H), 7.61-7.54 (m, 1H), 7.53-7.46 (m, 1H), 7.37-7.28 (m, 2H), 7.23 (d, J=8.8 Hz, 1H), 7.13-7.04 (m, 2H), 7.04-6.98 (m, 1H), 6.98-6.90 (m, 1H), 5.41-5.20 (m, 2H), 3.80-3.70 (m, 2H), 3.66-3.49 (m, 3H), 3.43-3.38 (m, 2H), 3.21 (s, 4H), 2.76-2.64 (m, 5H), 2.57-2.53 (m, 3H), 2.44-2.35 (m, 2H), 1.83-1.72 (m, 2H), 1.54-1.38 (m, 10H), 1.29-1.18 (m, 2H); [M+H]⁺=927.7.

### Example 105: 3-(tert-butyl)-N-(4-(5-(6-(1-((1-(4-(2,4-dioxotetrahydropyrimidin-1(2H)-yl)-3-methylphenyl)piperidin-4-yl)methyl)piperidin-4-yl)pyridin-3-yl)-1H-pyrazolo[3,4-b]pyridin-3-yl)-2-methylbenzyl)-1,2,4-oxadiazole-5-carboxamide

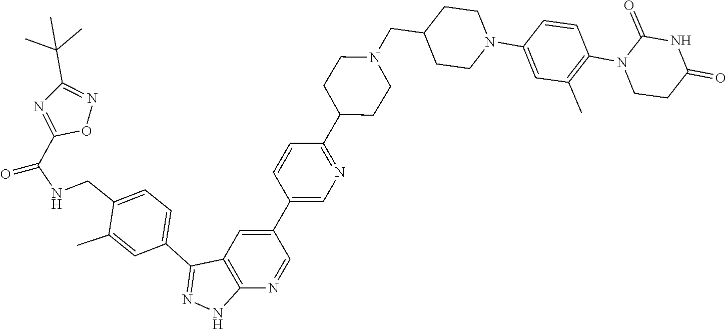 [SMILES: Cc1cc(-c2n[nH]c3ncc(-c4ccc(C5CCN(CC6CCN(c7ccc(N8CCC(=O)NC8=O)c(C)c7)CC6)CC5)nc4)cc23)ccc1CNC(=O)c1nc(C(C)(C)C)no1]

The titled compound was synthesized in the procedures similar to Example 1. ¹H NMR (400 MHz, DMSO) δ_{H} 13.93 (s, 1H), 10.26 (s, 1H), 9.88 (s, 1H), 8.98 (s, 1H), 8.91 (s, 1H), 8.80 (s, 1H), 8.20 (s, 1H), 7.93 (s, 2H), 7.43 (s, 2H), 7.05 (d, J=8.4 Hz, 1H), 6.83-6.80 (m, 2H), 4.53 (s, 2H), 3.70 (brs, 3H), 3.48 (brs, 1H), 3.23-2.59 (m, 9H), 2.46 (s, 3H), 2.35-1.65 (m, 11H), 1.37 (s, 9H), 1.24 (brs, 3H); [M+H]⁺=850.5.

### Example 106: 3-(tert-butyl)-N-(4-(5-(6-(1-((1-(4-(2,4-dioxotetrahydropyrimidin-1(2H)-yl)phenyl)piperidin-4-yl)methyl)piperidin-4-yl)pyridin-3-yl)-1H-pyrazolo[3,4-b]pyridin-3-yl)-2-methylbenzyl)-1,2,4-oxadiazole-5-carboxamide

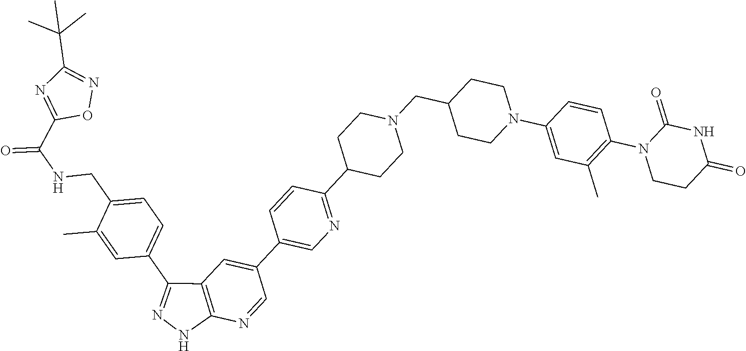 [SMILES: Cc1cc(-c2n[nH]c3ncc(-c4ccc(C5CCN(CC6CCN(c7ccc(N8CCC(=O)NC8=O)c(C)c7)CC6)CC5)nc4)cc23)ccc1CNC(=O)c1nc(C(C)(C)C)no1]

The titled compound was synthesized in the procedures similar to Example 1. ¹H NMR (400 MHz, DMSO) δ_{H} 13.93 (s, 1H), 10.26 (s, 1H), 9.88 (s, 1H), 8.98 (s, 1H), 8.91 (s, 1H), 8.80 (s, 1H), 8.20 (s, 1H), 7.93 (s, 2H), 7.43 (s, 2H), 7.05 (d, J=8.4 Hz, 1H), 6.83-6.80 (m, 2H), 4.53 (s, 2H), 3.70 (brs, 3H), 3.48 (brs, 1H), 3.23-2.59 (m, 9H), 2.46 (s, 3H), 2.35-1.65 (m, 11H), 1.37 (s, 9H), 1.24 (brs, 3H); [M+H]⁺=850.5.

### Example 107: 5-(tert-butyl)-N-(4-(5-(4-(4-(4-(2,6-dioxopiperidin-3-yl)phenethyl)piperazin-1-yl)phenyl)-1H-pyrazolo[3,4-b]pyridin-3-yl)-2-methylbenzyl)-1,2,4-oxadiazole-3-carboxamide

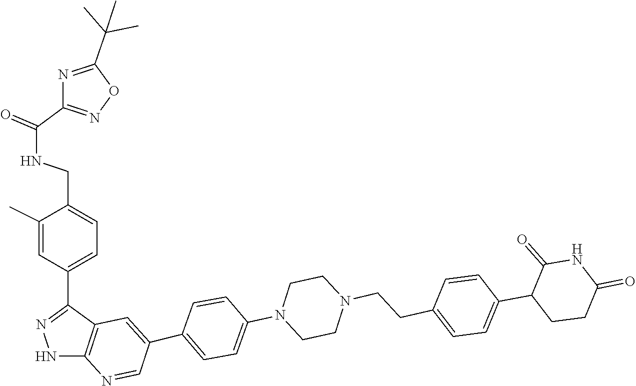 [SMILES: Cc1cc(-c2n[nH]c3ncc(-c4ccc(N5CCN(CCc6ccc(C7CCC(=O)NC7=O)cc6)CC5)cc4)cc23)ccc1CNC(=O)c1noc(C(C)(C)C)n1]

The titled compound was synthesized in the procedures similar to Example 1. ¹H NMR (400 MHz, DMSO) δ_{H} 13.78 (s, 1H), 10.83 (s, 1H), 9.47 (s, 1H), 8.82 (s, 1H), 8.58 (s, 1H), 7.90 (d, J=12.0 Hz, 2H), 7.69 (d, J=7.6 Hz, 2H), 7.40 (d, J=7.2 Hz, 1H), 7.22 (d, J=7.2 Hz, 2H), 7.14 (d, J=7.2 Hz, 2H), 7.07 (d, J=8.0 Hz, 2H), 4.52 (s, 2H), 3.87-3.75 (m, 1H), 3.23 (s, 4H), 2.78 (brs, 2H), 2.72-2.54 (m, 8H), 2.45 (s, 3H), 2.18 (brs, 1H), 2.04 (brs, 1H), 1.44 (s, 9H); [M+H]⁺=766.8.

### Example 108: 1-(tert-butyl)-N-(4-(5-(4-(4-(4-(2,6-dioxopiperidin-3-yl)phenethyl)piperazin-1-yl)phenyl)-1H-pyrazolo[3,4-b]pyridin-3-yl)-2-methylbenzyl)-1H-1,2,3-triazole-4-carboxamide

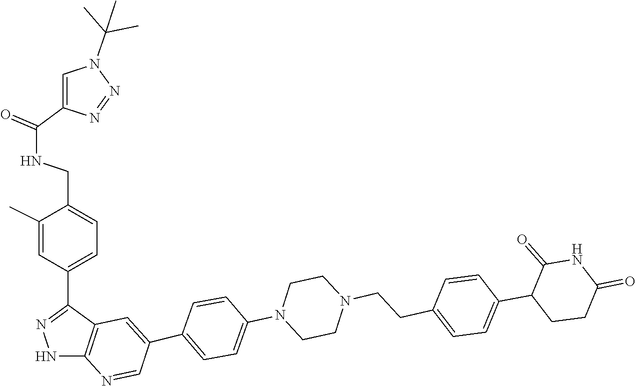 [SMILES: Cc1cc(-c2n[nH]c3ncc(-c4ccc(N5CCN(CCc6ccc(C7CCC(=O)NC7=O)cc6)CC5)cc4)cc23)ccc1CNC(=O)c1cn(C(C)(C)C)nn1]

The titled compound was synthesized in the procedures similar to Example 1. ¹H NMR (400 MHz, DMSO) δ_{H} 13.76 (s, 1H), 10.83 (s, 1H), 9.01 (s, 1H), 8.82 (s, 1H), 8.72 (s, 1H), 8.57 (s, 1H), 7.87 (s, 2H), 7.69 (d, J=8.0 Hz, 2H), 7.38 (d, J=7.2 Hz, 1H), 7.22 (s, 2H), 7.15 (s, 2H), 7.08 (s, 2H), 4.52 (s, 2H), 3.81 (brs, 1H), 3.22 (s, 4H), 2.85-2.50 (m, 10H), 2.45 (s, 3H), 2.17 (brs, 1H), 2.05 (brs, 1H), 1.65 (s, 9H); [M+H]⁺=765.9.

### Example 109: (R)—N-(1-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylphenyl)ethyl)-5-(1-methylcyclopropyl)-1,2,4-oxadiazole-3-carboxamide

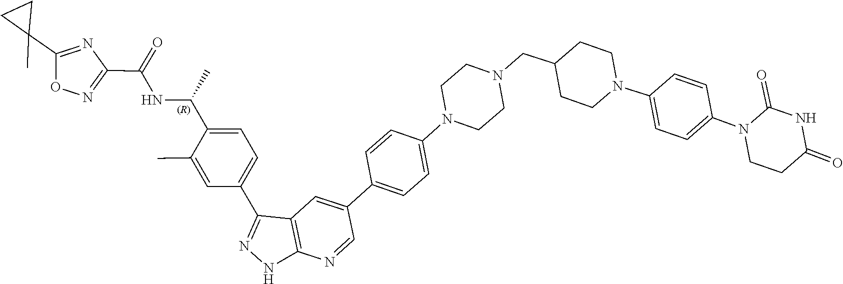

The titled compound was synthesized in the procedures similar to Example 1. ¹H NMR (400 MHz, DMSO) δ_{H} 13.77 (s, 1H), 10.27 (s, 1H), 9.49-9.38 (m, 1H), 8.82 (s, 1H), 8.58 (s, 1H), 7.93-7.87 (m, 1H), 7.84 (s, 1H), 7.69 (d, J=7.6 Hz, 2H), 7.64-7.54 (m, 1H), 7.14 (d, J=8.0 Hz, 2H), 7.07 (d, J=6.4 Hz, 2H), 6.98-6.88 (m, 2H), 5.42-5.29 (m, 1H), 3.70 (s, 4H), 3.22 (s, 4H), 2.73-2.65 (m, 4H), 2.61-2.53 (m, 7H), 2.29-2.19 (m, 2H), 1.90-1.79 (m, 2H), 1.78-1.67 (m, 1H), 1.57-1.46 (m, 6H), 1.41-1.32 (m, 2H), 1.31-1.20 (m, 2H), 1.19-1.13 (m, 2H); [M+H]⁺=848.9.

### Example 111 and 112: 5-(tert-butyl)-N—((R)-1-(4-(5-(4-(4-(4-((R)-2,6-dioxopiperidin-3-yl)phenethyl)piperazin-1-yl)phenyl)-1H-pyrazolo[3,4-b]pyridin-3-yl)-2-methylphenyl)ethyl)-1,2,4-oxadiazole-3-carboxamide and 5-(tert-butyl)-N—((R)-1-(4-(5-(4-(4-(4-((S)-2,6-dioxopiperidin-3-yl)phenethyl)piperazin-1-yl)phenyl)-1H-pyrazolo[3,4-b]pyridin-3-yl)-2-methylphenyl)ethyl)-1,2,4-oxadiazole-3-carboxamide

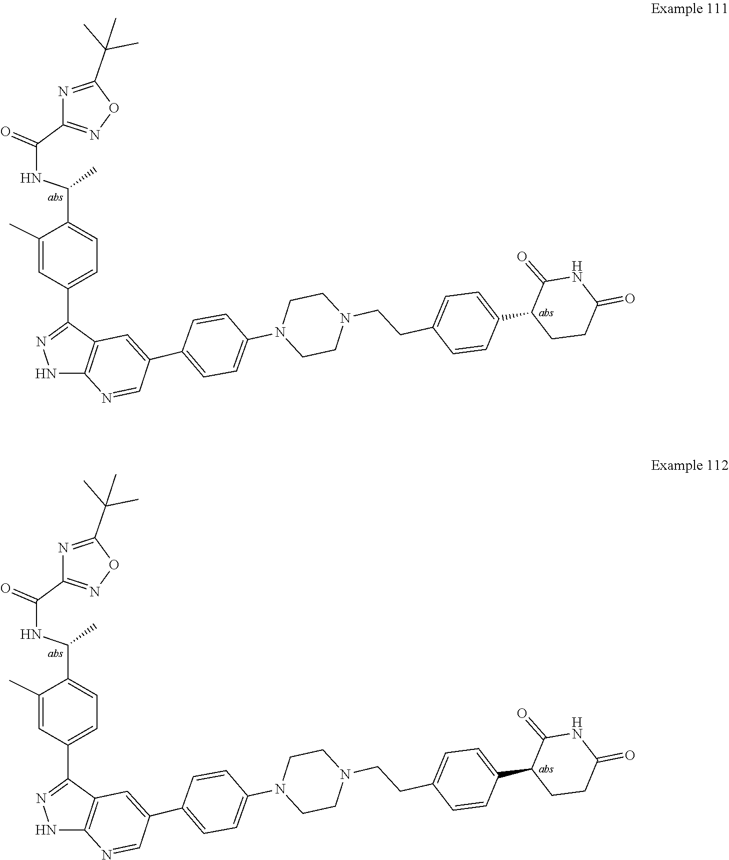 [SMILES: Cc1cc(-c2n[nH]c3ncc(-c4ccc(N5CCN(CCc6ccc([C@H]7CCC(=O)NC7=O)cc6)CC5)cc4)cc23)ccc1[C@@H](C)NC(=O)c1noc(C(C)(C)C)n1 ; Cc1cc(-c2n[nH]c3ncc(-c4ccc(N5CCN(CCc6ccc([C@@H]7CCC(=O)NC7=O)cc6)CC5)cc4)cc23)ccc1[C@@H](C)NC(=O)c1noc(C(C)(C)C)n1]

The Example 35 (500 mg) was separated by Prep-Chiral-HPLC with following conditions: Column: (R, R)-WHELK-O1-Kromasil, 2.12*25 cm, 5 μm; Mobile Phase A: MTBE (0.5% 2 M NH₃-MeOH)—HPLC, Mobile Phase B: MeOH; Flow rate: 20 mL/min; Gradient: 50% B to 50% B in 19 min; Detector: 220/254 nm; RT1: 11.904 min; RT2: 15.819 min; Sample Solvent: MeOH:DCM=1: 1; Injection Volume: 0.8 mL; Number Of Runs: 8; This resulted in Example 111 (RT1: 11.904 min, 185.6 mg, 37.1%) and Example 112 (RT2: 15.819 min, 202.1 mg, 40.4%). Example 111: ¹H NMR (400 MHz, DMSO) δ_{H} 13.77 (s, 1H), 10.83 (s, 1H), 9.50 (s, 1H), 8.82 (s, 1H), 8.58 (s, 1H), 7.96-7.82 (m, 2H), 7.73-7.57 (m, 3H), 7.28-7.02 (m, 6H), 5.36 (s, 1H), 3.83 (s, 1H), 3.23 (s, 3H), 2.82-2.56 (m, 13H), 2.25-1.96 (m, 2H), 1.51 (d, J=6.4 Hz, 3H), 1.42 (s, 9H); [M+H]⁺=780.8. Example 112: ¹H NMR (400 MHz, DMSO) δ_{H} 13.77 (s, 1H), 10.83 (s, 1H), 9.50 (s, 1H), 8.82 (s, 1H), 8.58 (s, 1H), 7.96-7.82 (m, 2H), 7.73-7.57 (m, 3H), 7.28-7.02 (m, 6H), 5.36 (s, 1H), 3.83 (s, 1H), 3.23 (s, 3H), 2.82-2.56 (m, 13H), 2.25-1.96 (m, 2H), 1.51 (d, J=6.4 Hz, 3H), 1.42 (s, 9H); [M+H]⁺=780.8.

### Example 113 and 114: 5-(tert-butyl)-N—((R)-1-(4-(5-(4-(4-(4-((R)-2,6-dioxopiperidin-3-yl)-3-fluorophenethyl)piperazin-1-yl)phenyl)-1H-pyrazolo[3,4-b]pyridin-3-yl)-2-methylphenyl)ethyl)-1,2,4-oxadiazole-3-carboxamide and 5-(tert-butyl)-N—((R)-1-(4-(5-(4-(4-(4-((S)-2,6-dioxopiperidin-3-yl)-3-fluorophenethyl)piperazin-1-yl)phenyl)-1H-pyrazolo[3,4-b]pyridin-3-yl)-2-methylphenyl)ethyl)-1,2,4-oxadiazole-3-carboxamide

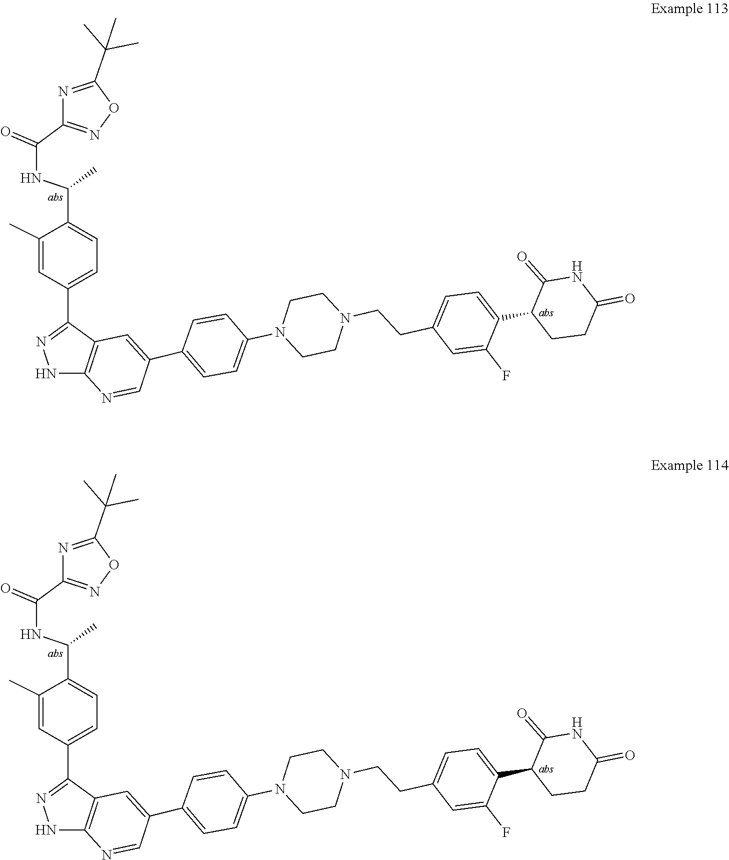 [SMILES: Cc1cc(-c2n[nH]c3ncc(-c4ccc(N5CCN(CCc6ccc([C@H]7CCC(=O)NC7=O)c(F)c6)CC5)cc4)cc23)ccc1[C@@H](C)NC(=O)c1noc(C(C)(C)C)n1 ; Cc1cc(-c2n[nH]c3ncc(-c4ccc(N5CCN(CCc6ccc([C@@H]7CCC(=O)NC7=O)c(F)c6)CC5)cc4)cc23)ccc1[C@@H](C)NC(=O)c1noc(C(C)(C)C)n1]

The Example 36 (280 mg) was separated by Prep-Chiral-HPLC with following conditions: Column: (R, R)-WHELK-O1-Kromasil, 2.12*25 cm, 5 μm; Mobile Phase A: MTBE (0.5% 2 M NH₃-MeOH)—HPLC, Mobile Phase B: EtOH; Flow rate: 20 mL/min; Gradient: 50% B to 50% B in 22 min; Detector: 220/254 nm; RT1: 14.615 min; RT2: 18.411 min; Sample Solvent: EtOH:DCM=1:1; Injection Volume: 1 mL; Number Of Runs: 7; This resulted in Example 113 (RT1: 14.615 min, 116.3 mg, 41.4%) and Example 114 (RT2: 18.411 min, 107.8 mg, 38.5%). Example 113: ¹H NMR (400 MHz, DMSO) δ_{H} 13.77 (s, 1H), 10.88 (s, 1H), 9.49 (d, J=6.8 Hz, 1H), 8.82 (s, 1H), 8.58 (s, 1H), 7.96-7.81 (m, 2H), 7.75-7.56 (m, 3H), 7.27-7.02 (m, 5H), 5.41-5.31 (m, 1H), 4.07-4.02 (m, 1H), 3.22 (s, 4H), 2.86-2.54 (m, 13H), 2.27-2.12 (m, 1H), 2.04-1.99 (m, 1H), 1.51 (d, J=6.3 Hz, 3H), 1.42 (s, 9H); [M+H]⁺=798.8. Example 114: ¹H NMR (400 MHz, DMSO) δ_{H}13.77 (s, 1H), 10.88 (s, 1H), 9.49 (d, J=6.8 Hz, 1H), 8.82 (s, 1H), 8.58 (s, 1H), 7.96-7.81 (m, 2H), 7.75-7.56 (m, 3H), 7.27-7.02 (m, 5H), 5.41-5.31 (m, 1H), 4.07-4.02 (m, 1H), 3.22 (s, 4H), 2.86-2.54 (m, 13H), 2.27-2.12 (m, 1H), 2.04-1.99 (m, 1H), 1.51 (d, J=6.3 Hz, 3H), 1.42 (s, 9H); [M+H]⁺=798.8.

### Example 115: (R)-5-(tert-butyl)-N-(1-(4-(5-(4-(4-((1-(3-chloro-4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

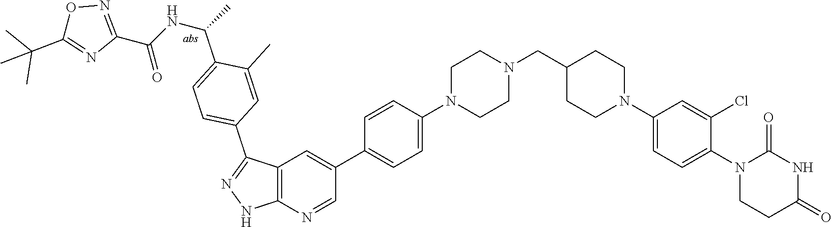 [SMILES: Cc1cc(-c2n[nH]c3ncc(-c4ccc(N5CCN(CC6CCN(c7ccc(N8CCC(=O)NC8=O)c(Cl)c7)CC6)CC5)cc4)cc23)ccc1[C@@H](C)NC(=O)c1noc(C(C)(C)C)n1]

The titled compound was synthesized in the procedures similar to Example 1. ¹H NMR (400 MHz, CD₃OD) δ_{H} 8.77 (s, 1H), 8.52 (s, 1H), 7.81 (s, 2H), 7.63 (d, J=8.8 Hz, 3H), 7.24 (d, J=8.9 Hz, 1H), 7.17-7.04 (m, 3H), 6.95 (d, J=8.5 Hz, 1H), 5.55-5.45 (m, 1H), 3.80 (d, J=11.6 Hz, 2H), 3.73-3.69 (m, 2H), 3.51-3.36 (m, 3H), 3.19-2.99 (m, 3H), 2.90-2.68 (m, 7H), 2.56 (s, 3H), 2.04-1.87 (m, 3H), 1.61 (d, J=6.4 Hz, 3H), 1.51-1.25 (m, 12H); [M+H]⁺=884.8.

### Example 116: 5-(tert-butyl)-N-((1R)-1-(4-(5-(5-(4-(4-(2,6-dioxopiperidin-3-yl)phenethyl)piperazin-1-yl)pyridin-2-yl)-1H-pyrazolo[3,4-b]pyridin-3-yl)-2-methylphenyl)ethyl)-1,2,4-oxadiazole-3-carboxamide

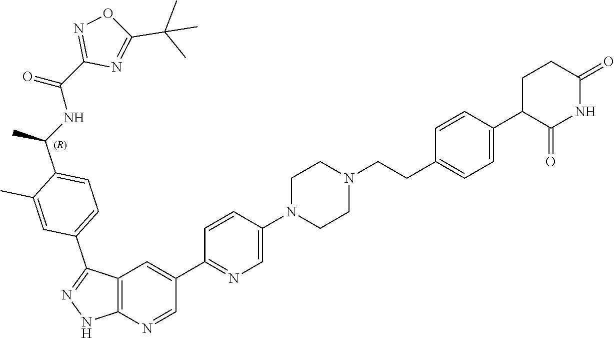

### Step 1: N-((1R)-1-(4-(5-bromo-1-(tetrahydro-2H-pyran-2-yl)-1H-pyrazolo[3,4-b]pyridin-3-yl)-2-methylphenyl)ethyl)-5-(tert-butyl)-1,2,4-oxadiazole-3-carboxamide

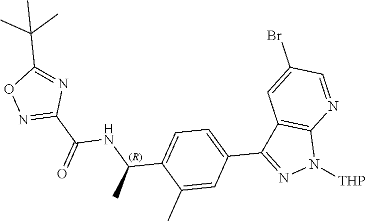

To a solution of (R)-5-(tert-butyl)-N-(1-(2-methyl-4-(4,4,5,5-tetramethyl-1,3,2-dioxaborolan-2-yl)phenyl)ethyl)-1,2,4-oxadiazole-3-carboxamide (2.0 g, 4.83 mmol) in 1,4-dioxane (50 mL) and H₂O (5 mL) were added 5-bromo-3-iodo-1-(tetrahydro-2H-pyran-2-yl)-1H-indazole (1.97 g, 4.83 mmol), Pd(dppf)Cl₂ (353 mg, 0.483 mmol) and K₂CO₃ (1.3 g, 9.66 mmol). The mixture was stirred overnight at 85° C. under N₂. The solvent was removed under reduced pressure and the crude product was purified with silica gel column chromatography (PE:EtOAc=10:1˜2:1 gradient elution) to give the product (2.4 g, 82.8%). [M+H]⁺=567.2.

### Step 2: 5-(tert-butyl)-N-((1R)-1-(2-methyl-4-(1-(tetrahydro-2H-pyran-2-yl)-5-(4,4,5,5-tetramethyl-1,3,2-dioxaborolan-2-yl)-1H-pyrazolo[3,4-b]pyridin-3-yl)phenyl)ethyl)-1,2,4-oxadiazole-3-carboxamide

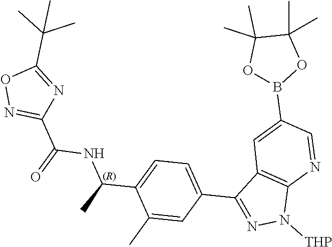

To a solution of N-((1R)-1-(4-(5-bromo-1-(tetrahydro-2H-pyran-2-yl)-1H-pyrazolo[3,4-b]pyridin-3-yl)-2-methylphenyl)ethyl)-5-(tert-butyl)-1,2,4-oxadiazole-3-carboxamide (4.4 g, 7.73 mmol) in 1,4-dioxane (100 mL) were added 4,4,4′,4′,5,5,5′,5′-octamethyl-2,2′-bi(1,3,2-dioxaborolane) (3.9 g, 15.5 mmol), Pd(dppf)Cl₂ (566 mg, 0.773 mmol) and KOAc (1.52 g, 15.5 mmol). The mixture was stirred overnight at 100° C. under N₂. The solvent was removed under reduced pressure and the crude product was purified with silica gel column chromatography (PE:EtOAc=10:1˜2:1 gradient elution) to give the product (3.8 g, 80.0%). [M+H]⁺=615.3.

### Step 3: tert-butyl 4-(6-(3-(4-((R)-1-(5-(tert-butyl)-1,2,4-oxadiazole-3-carboxamido)ethyl)-3-methylphenyl)-1-(tetrahydro-2H-pyran-2-yl)-1H-pyrazolo[3,4-b]pyridin-5-yl)pyridin-3-yl)piperazine-1-carboxylate

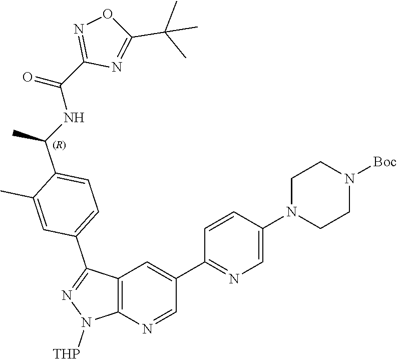

To a solution of tert-butyl 4-(6-bromopyridin-3-yl)piperazine-1-carboxylate (1.2 g, 3.5 mmol) in 1,4-dioxane (80 mL) and H₂O (20 mL) were added X-Phos (166 mg, 0.35 mmol), Pd-G3 (280 mg, 0.35 mmol) and K₃PO₄ (1484 mg, 7.0 mmol). A solution of 5-(tert-butyl)-N-((1R)-1-(2-methyl-4-(1-(tetrahydro-2H-pyran-2-yl)-5-(4,4,5,5-tetramethyl-1,3,2-dioxaborolan-2-yl)-1H-pyrazolo[3,4-b]pyridin-3-yl)phenyl)ethyl)-1,2,4-oxadiazole-3-carboxamide (2.16 g, 3.5 mmol) in 1,4-dioxane (80 mL) was added dropwise at 90° C. under N₂. The mixture was stirred overnight at 100° C. under N₂. The solvent was removed under reduced pressure and the crude product was purified with silica gel column chromatography (PE:EtOAc=1:1˜0:1 gradient elution) to give the product (430 mg, 17%). [M+H]⁺=750.4.

### Step 4: (R)-5-(tert-butyl)-N-(1-(2-methyl-4-(5-(5-(piperazin-1-yl)pyridin-2-yl)-1H-pyrazolo[3,4-b]pyridin-3-yl)phenyl)ethyl)-1,2,4-oxadiazole-3-carboxamide

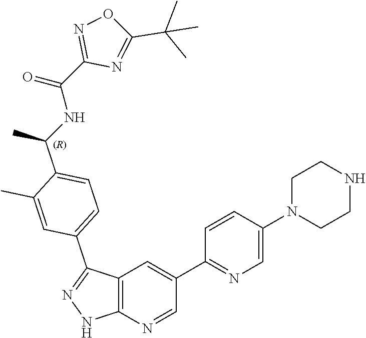

To a solution of tert-butyl 4-(6-(3-(4-((R)-1-(5-(tert-butyl)-1,2,4-oxadiazole-3-carboxamido)ethyl)-3-methylphenyl)-1-(tetrahydro-2H-pyran-2-yl)-1H-pyrazolo[3,4-b]pyridin-5-yl)pyridin-3-yl)piperazine-1-carboxylate (430 mg, 0.57 mmol) in DCM (10 mL) was added TFA (10 mL). The mixture was stirred overnight at rt. The solvent was removed under vacuo to give the crude product which was further purified by pre-HPLC to give the product (180 mg, 56%). [M+H]⁺=566.3.

### Step 5: 5-(tert-butyl)-N-((1R)-1-(4-(5-(5-(4-(4-(2,6-dioxopiperidin-3-yl)phenethyl)piperazin-1-yl)pyridin-2-yl)-1H-pyrazolo[3,4-b]pyridin-3-yl)-2-methylphenyl)ethyl)-1,2,4-oxadiazole-3-carboxamide

To a solution of (R)-5-(tert-butyl)-N-(1-(2-methyl-4-(5-(5-(piperazin-1-yl)pyridin-2-yl)-1H-pyrazolo[3,4-b]pyridin-3-yl)phenyl)ethyl)-1,2,4-oxadiazole-3-carboxamide (50 mg, 0.0883 mmol) in DCM (20 mL) and MeOH (5 mL) were added 2-(4-(2,6-dioxopiperidin-3-yl)phenyl)acetaldehyde (25 mg, 0.106 mmol) and AcOH (0.04 mL). The mixture was stirred overnight at room temperature under N₂. To the mixture was added NaBH(OAc)₃ (37 mg, 0.177 mmol), then stirred for another 3 h at room temperature. The reaction was quenched by NaHCO₃ (aq, 20 mL) and then extracted with DCM (30 mL×3). The organic layer was dried over with Na₂SO₄, filtered and concentrated to give the crude product which was purified by pre-TLC to give the product (33 mg, 48%). ¹H NMR (400 MHz, DMSO) δ_{H} 13.84 (s, 1H), 10.84 (s, 1H), 9.50 (d, J=8.0 Hz, 1H), 9.24 (s, 1H), 8.94 (s, 1H), 8.47 (s, 1H), 8.05 (s, 1H), 7.90-7.84 (m, 2H), 7.63 (d, J=8.0 Hz, 1H), 7.51-7.47 (m, 1H), 7.23-7.15 (m, 4H), 5.37 (s, 1H), 3.82 (d, J=8.0 Hz, 1H), 3.32-3.29 (m, 4H), 2.80-2.78 (m, 3H), 2.64-2.56 (m, 7H), 2.46 (s, 3H), 2.19-2.16 (m, 1H), 2.04 (s, 1H), 1.53 (s, 3H), 1.43 (s, 9H); [M+H]⁺=781.8.

### Example 117: 5-(tert-butyl)-N-((1R)-1-(4-(5-(6-(1-(4-(2,6-dioxopiperidin-3-yl)phenethyl)piperidin-4-yl)pyridin-3-yl)-1H-pyrazolo[3,4-b]pyridin-3-yl)-2-methylphenyl)ethyl)-1,2,4-oxadiazole-3-carboxamide

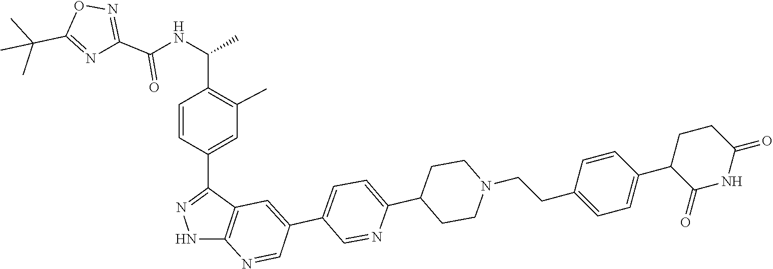 [SMILES: Cc1cc(-c2n[nH]c3ncc(-c4ccc(C5CCN(CCc6ccc(C7CCC(=O)NC7=O)cc6)CC5)nc4)cc23)ccc1[C@@H](C)NC(=O)c1noc(C(C)(C)C)n1]

### Step 1: tert-butyl 5-chloro-3′,6′-dihydro-[2,4′-bipyridine]-1′(2′H)-carboxylate

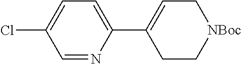 [SMILES: CC(C)(C)OC(=O)N1CC=C(c2ccc(Cl)cn2)CC1]

To a solution of 2-bromo-5-chloropyridine (3.8 g, 20 mmol) in dioxane/H₂O (5:1, 120 mL) were added tert-butyl 4-(4,4,5,5-tetramethyl-1,3,2-dioxaborolan-2-yl)-3,6-dihydropyridine-1(2H)-carboxylate (6.2 g, 20 mmol), Pd(dppf)Cl₂·DCM (818 mg, 1 mmol) and Na₂CO₃ (6.4 g, 60 mmol). After stirring at 100° C. for 5 hs, the solvent was evaporated. To the residue was added water (100 mL) and extracted with EtOAc (100 mL*2). The organic phase was combined and purified by flash chromatography with PE/EtOAc (100:1 to 10:1) to give the product (4.7 g, 79.7%). ¹H NMR (400 MHz, CDCl₃) δ_{H} 8.50 (s, 1H), 7.69-7.52 (m, 1H), 7.32 (d, J=8.4 Hz, 1H), 6.60 (s, 1H), 4.13 (s, 2H), 3.64 (s, 2H), 2.61 (s, 2H), 1.49 (s, 9H).

### Step 2: tert-butyl 5-(4,4,5,5-tetramethyl-1,3,2-dioxaborolan-2-yl)-3′,6′-dihydro-[2,4′-bipyridine]-1′(2′H)-carboxylate

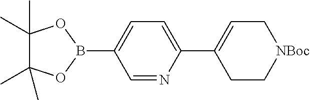 [SMILES: CC(C)(C)OC(=O)N1CC=C(c2ccc(B3OC(C)(C)C(C)(C)O3)cn2)CC1]

To a solution of tert-butyl 5-chloro-3′,6′-dihydro-[2,4′-bipyridine]-1′(2′H)-carboxylate (0.7 g, 2.38 mmol) in dioxane (40 mL) were added B₂pin₂ (1.5 g, 5.9 mmol), Pd₂(dba)₃ (220 mg, 0.24 mmol), XPhos (230 mg, 0.48 mmol) and AcOK (697 mg, 7.11 mmol). After stirring at 100° C. for 5 hs, the mixture was evaporated and purified by flash chromatography with DCM/MeOH (100:1 to 20:1) to give the product (800 mg, 87.4%). ¹H NMR (400 MHz, CDCl₃) δ 8.89 (s, 1H), 8.02 (d, J=7.2 Hz, 1H), 7.35 (d, J=7.6 Hz, 1H), 6.69 (s, 1H), 4.15 (s, 2H), 3.65 (s, 2H), 2.65 (s, 2H), 1.49 (s, 9H), 1.35 (s, 12H).

### Step 3: tert-butyl 4-(5-(4,4,5,5-tetramethyl-1,3,2-dioxaborolan-2-yl)pyridin-2-yl)piperidine-1-carboxylate

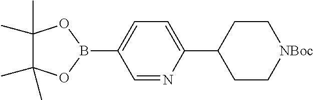 [SMILES: CC(C)(C)OC(=O)N1CCC(c2ccc(B3OC(C)(C)C(C)(C)O3)cn2)CC1]

A solution of tert-butyl 5-(4,4,5,5-tetramethyl-1,3,2-dioxaborolan-2-yl)-3′,6′-dihydro-[2,4′-bipyridine]-1′(2′H)-carboxylate (2.5 g, 6.5 mmol) with Pd/C (0.25 g) in MeOH/THF (3:1, 60 mL) was stirred at 20-30° C. for 18 hs. The solid was filtered off and the filtrate was concentrated and used for next step directly.

### Step 4: tert-butyl 4-(5-(3-(4-((R)-1-(5-(tert-butyl)-1,2,4-oxadiazole-3-carboxamido)ethyl)-3-methylphenyl)-1-(tetrahydro-2H-pyran-2-yl)-1H-pyrazolo[3,4-b]pyridin-5-yl)pyridin-2-yl)piperidine-1-carboxylate

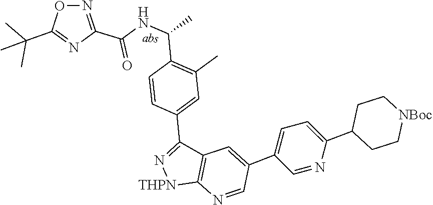

To a solution of 5-bromo-3-iodo-1-(tetrahydro-2H-pyran-2-yl)-1H-pyrazolo[3,4-b]pyridine (0.6 g, 1.5 mmol) in dioxane/H₂O (5:1, 40 mL) were added (R)-5-(tert-butyl)-N-(1-(2-methyl-4-(4,4,5,5-tetramethyl-1,3,2-dioxaborolan-2-yl)phenyl)ethyl)-1,2,4-oxadiazole-3-carboxamide (0.6 g, 1.5 mmol), K₂CO₃ (0.6 g, 4.5 mmol) and Pd(dppf)Cl₂·DCM (0.12 g, 0.15 mmol). After stirring at 80° C. for 18 hs, tert-butyl 4-(5-(4,4,5,5-tetramethyl-1,3,2-dioxaborolan-2-yl)pyridin-2-yl)piperidine-1-carboxylate (0.57 g, 1.5 mmol) was added. The mixture was stirred at 90° C. for another 6 hs, and then the solvent was evaporated. To the residue was added H₂O (40 mL) and the mixture was extracted with EtOAc (40 mL*2). The organic phase was concentrated and purified by pre-TLC with PE/EtOAc (1:3) to give the product (0.5 g, 45.4%). [M+H]⁺=749.6.

### Step 5: (R)-5-(tert-butyl)-N-(1-(2-methyl-4-(5-(6-(piperidin-4-yl)pyridin-3-yl)-1H-pyrazolo[3,4-b]pyridin-3-yl)phenyl)ethyl)-1,2,4-oxadiazole-3-carboxamide hydrochloride

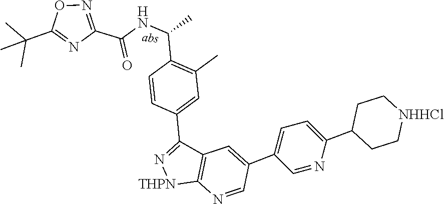

To a solution of tert-butyl 4-(5-(3-(4-((R)-1-(5-(tert-butyl)-1,2,4-oxadiazole-3-carboxamido)ethyl)-3-methylphenyl)-1-(tetrahydro-2H-pyran-2-yl)-1H-pyrazolo[3,4-b]pyridin-5-yl)pyridin-2-yl)piperidine-1-carboxylate (0.5 g, 0.67 mmol) in DCM (5 mL) was added HCl/dioxane (4 N, 30 mL). The mixture was stirred at 20-30° C. for 2 hs, concentrated and used for next step directly without further operation.

### Step 6: 5-(tert-butyl)-N-((1R)-1-(4-(5-(6-(1-(4-(2,6-dioxopiperidin-3-yl)phenethyl)piperidin-4-yl)pyridin-3-yl)-1H-pyrazolo[3,4-b]pyridin-3-yl)-2-methylphenyl)ethyl)-1,2,4-oxadiazole-3-carboxamide

To a solution of (R)-5-(tert-butyl)-N-(1-(2-methyl-4-(5-(6-(piperidin-4-yl)pyridin-3-yl)-1H-pyrazolo[3,4-b]pyridin-3-yl)phenyl)ethyl)-1,2,4-oxadiazole-3-carboxamide hydrochloride (120 mg, 0.2 mmol) in DCM/EtOH (5:1, 30 mL) were added 2-(4-(2,6-dioxopiperidin-3-yl)phenyl)acetaldehyde (46 mg, 0.2 mmol) and NaOAc (49 mg, 0.6 mmol). After stirring for 30 min, NaBH(OAc)₃ (130 mg, 0.6 mmol) was added. The reaction was stirred for another 3 hs. The solvent was evaporated and purified by pre-TLC with DCM/MeOH (10:1) to give the product (26 mg, 16.7%). ¹H NMR (400 MHz, DMSO) δ_{H} 13.92 (s, 1H), 10.85 (s, 1H), 9.51 (d, J=7.2 Hz, 1H), 9.00 (s, 1H), 8.91 (s, 1H), 8.80 (s, 1H), 8.22 (d, J=7.2 Hz, 1H), 7.95 (d, J=7.2 Hz, 1H), 7.88 (s, 1H), 7.60 (d, J=8.0 Hz, 1H), 7.46 (d, J=7.2 Hz, 1H), 7.24 (s, 2H), 7.19 (s, 2H), 5.36 (s, 1H), 3.84 (d, J=9.2 Hz, 1H), 2.92 (s, 5H), 2.67 (s, 2H), 2.50 (brs, 7H), 2.26-1.92 (m, 6H), 1.51 (d, J=5.2 Hz, 3H), 1.42 (s, 9H); [M+H]⁺=780.4.

### Example 118: (R)-3-(tert-butyl)-N-(1-(4-(5-(4-(4-(3-(4-(2,4-dioxotetrahydropyrimidin-1(2H)-yl)-3-methoxyphenoxy)propyl)piperazin-1-yl)phenyl)-1H-pyrazolo[3,4-b]pyridin-3-yl)-2-methylphenyl)ethyl)-1,2,4-oxadiazole-5-carboxamide

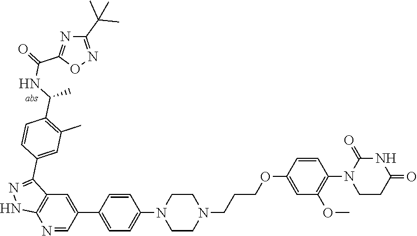 [SMILES: COc1cc(OCCCN2CCN(c3ccc(-c4cnc5[nH]nc(-c6ccc([C@@H](C)NC(=O)c7nc(C(C)(C)C)no7)c(C)c6)c5c4)cc3)CC2)ccc1N1CCC(=O)NC1=O]

The titled compound was synthesized in the procedures similar to Example 1. ¹H NMR (400 MHz, DMSO) δ_{H} 13.78 (s, 1H), 10.25 (s, 1H), 9.52-9.47 (m, 1H), 8.83 (s, 1H), 8.59 (s, 1H), 7.95-7.88 (m, 1H), 7.85 (s, 1H), 7.75-7.65 (m, 2H), 7.63-7.58 (m, 1H), 7.16-7.05 (m, 3H), 6.65 (s, 1H), 6.58-6.52 (m, 1H), 5.40-5.30 (m, 1H), 4.08 (s, 3H), 3.79 (s, 4H), 3.55-3.48 (m, 2H), 3.28-3.14 (m, 4H), 2.70-2.55 (m, 6H), 2.00-1.90 (m, 2H), 1.54-1.48 (m, 3H), 1.42 (s, 9H); [M+H]⁺=841.8.

### Example 119: (R)-5-(tert-butyl)-N-(1-(4-(5-(4-(4-(4-(2,4-dioxotetrahydropyrimidin-1(2H)-yl)-3-methylphenethyl)piperazin-1-yl)phenyl)-1H-pyrazolo[3,4-b]pyridin-3-yl)-2-methylphenyl)ethyl)-1,2,4-oxadiazole-3-carboxamide

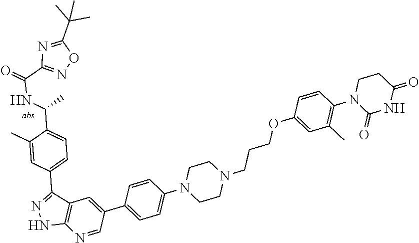 [SMILES: Cc1cc(-c2n[nH]c3ncc(-c4ccc(N5CCN(CCCOc6ccc(N7CCC(=O)NC7=O)c(C)c6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.78 (s, 1H), 10.33 (s, 1H), 9.51 (s, 1H), 8.83 (s, 1H), 8.59 (s, 1H), 7.91 (s, 1H), 7.85 (s, 1H), 7.70 (s, 2H), 7.61 (d, J=8.0 Hz, 1H), 7.22-7.05 (m, 6H), 5.36 (s, 1H), 3.85-3.64 (m, 2H), 3.56-3.44 (m, 2H), 3.25 (s, 4H), 2.89-2.61 (m, 9H), 2.23-2.13 (m, 4H), 1.55-1.48 (m, 3H), 1.42 (s, 9H); [M+H]⁺=795.8.

### Example 120: (R)-5-(tert-butyl)-N-(1-(4-(5-(4-(4-((1-(4-(2,4-dioxotetrahydropyrimidin-1(2H)-yl)phenyl)piperidin-4-yl)methyl)piperazin-1-yl)-3-methoxyphenyl)-1H-pyrazolo[3,4-b]pyridin-3-yl)-2-methylphenyl)ethyl)-1,2,4-oxadiazole-3-carboxamide

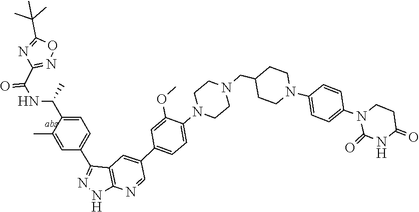 [SMILES: COc1cc(-c2cnc3[nH]nc(-c4ccc([C@@H](C)NC(=O)c5noc(C(C)(C)C)n5)c(C)c4)c3c2)ccc1N1CCN(CC2CCN(c3ccc(N4CCC(=O)NC4=O)cc3)CC2)CC1]

The titled compound was synthesized in the procedures similar to Example 1. ¹H NMR (400 MHz, DMSO) δ_{H} 13.85 (s, 1H), 10.27 (s, 1H), 9.53-9.48 (m, 1H), 8.87 (s, 1H), 8.65 (s, 1H), 7.92 (d, J=8.0 Hz, 1H), 7.86 (s, 1H), 7.61 (d, J=8.0 Hz, 1H), 7.42-7.30 (m, 1H), 7.18-7.10 (m, 1H), 7.09-6.90 (m, 3H), 5.40-5.30 (m, 1H), 3.96-3.88 (m, 1H), 3.76-3.66 (m, 4H), 3.65-3.48 (m, 2H), 3.30-3.18 (m, 2H), 3.16-2.98 (m, 3H), 2.76-2.64 (m, 4H), 2.28-2.20 (m, 1H), 2.10-2.00 (m, 1H), 1.98-1.89 (m, 1H), 1.87-1.65 (m, 2H), 1.51 (d, J=4.0 Hz, 3H), 1.42 (s, 9H); [M+H]⁺=880.8.

### Example 121: 5-(tert-butyl)-N-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-(hydroxymethyl)benzyl)-1,2,4-oxadiazole-3-carboxamide

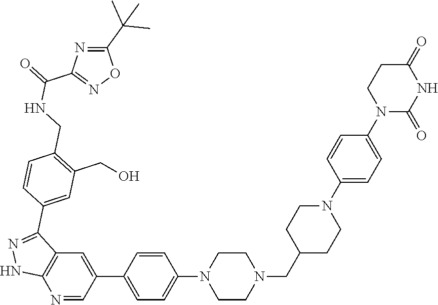 [SMILES: CC(C)(C)c1nc(C(=O)NCc2ccc(-c3n[nH]c4ncc(-c5ccc(N6CCN(CC7CCN(c8ccc(N9CCC(=O)NC9=O)cc8)CC7)CC6)cc5)cc34)cc2CO)no1]

The titled compound was synthesized in the procedures similar to Example 1. ¹H NMR (400 MHz, DMSO) δ_{H} 13.80 (s, 1H), 10.27 (s, 1H), 9.43 (s, 1H), 8.83 (s, 1H), 8.59 (s, 1H), 8.10 (s, 1H), 7.98 (d, J=8.0 Hz, 1H), 7.68 (d, J=8.0 Hz, 2H), 7.44 (d, J=8.0 Hz, 1H), 7.14 (d, J=8.0 Hz, 2H), 7.08 (d, J=8.0 Hz, 2H), 6.93 (d, J=8.0 Hz, 2H), 5.38 (s, 1H), 4.73 (s, 2H), 4.57 (s, 2H), 3.70 (s, 4H), 3.22 (s, 4H), 2.71-2.66 (m, 3H), 2.53 (s, 2H), 2.24 (s, 2H), 1.82 (d, J=12.0 Hz, 2H), 1.73 (s, 1H), 1.43 (s, 9H), 1.30-1.15 (m, 3H); [M+H]⁺=852.8.

### Example 122: (R)-5-(tert-butyl)-N-(1-(4-(5-(5-(4-(4-(2,4-dioxotetrahydropyrimidin-1(2H)-yl)phenethyl)piperazin-1-yl)pyridin-2-yl)-1H-pyrazolo[3,4-b]pyridin-3-yl)-2-methylphenyl)ethyl)-1,2,4-oxadiazole-3-carboxamide

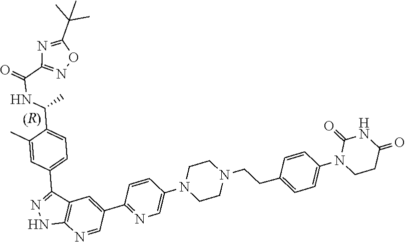

The titled compound was synthesized in the procedures similar to Example 1. ¹H NMR (400 MHz, DMSO) δ_{H} 13.83 (s, 1H), 10.35 (s, 1H), 9.49 (d, J=8.0 Hz, 1H), 9.23 (s, 1H), 8.93 (s, 1H), 8.45 (s, 1H), 8.04 (d, J=8.0 Hz, 1H), 7.90-7.83 (m, 2H), 7.63 (d, J=8.0 Hz, 1H), 7.46 (d, J=8.0 Hz, 1H), 7.30-7.23 (m, 4H), 5.37 (s, 1H), 3.78-3.73 (m, 4H), 3.31-3.29 (m, 4H), 2.82-2.77 (m, 3H), 2.73-2.60 (m, 7H), 2.46 (s, 3H), 2.41-2.33 (m, 2H), 1.51 (s, 3H), 1.43 (s, 9H); [M+H]⁺=782.5.

### Example 123: 5-(tert-butyl)-N-((1R)-1-(4-(5-(4-(4-(4-((2,6-dioxopiperidin-3-yl)oxy)phenethyl)piperazin-1-yl)phenyl)-1H-pyrazolo[3,4-b]pyridin-3-yl)-2-methylphenyl)ethyl)-1,2,4-oxadiazole-3-carboxamide

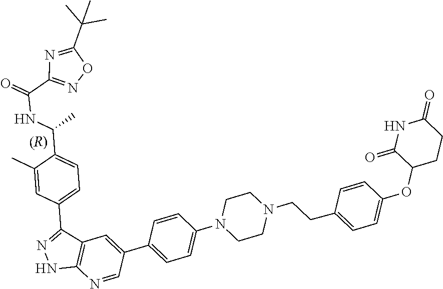

The titled compound was synthesized in the procedures similar to Example 1. ¹H NMR (400 MHz, DMSO) δ_{H} 13.78 (s, 1H), 10.93 (s, 1H), 9.49 (s, 1H), 9.24 (s, 1H), 8.82 (s, 1H), 8.58 (s, 1H), 7.93-7.85 (m, 2H), 7.69 (d, J=8.0 Hz, 2H), 7.60 (d, J=8.0 Hz, 1H), 7.18-7.08 (m, 4H), 6.94-6.93 (m, 2H), 5.36 (s, 1H), 5.17-5.13 (m, 1H), 3.22-3.19 (m, 4H), 2.74-2.72 (m, 3H), 2.68-2.61 (m, 7H), 2.46 (s, 3H), 2.18-2.13 (m, 2H), 1.50 (s, 3H), 1.42 (s, 9H); [M+H]⁺=796.8.

### Example 124: (R)-5-(tert-butyl)-N-(1-(4-(5-(4-(4-((1-(5-(2,4-dioxotetrahydropyrimidin-1(2H)-yl)pyridin-2-yl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

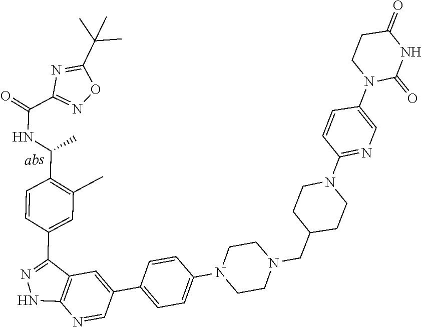

### Step 1: 1-(5-(2,4-dioxotetrahydropyrimidin-1(2H)-yl)pyridin-2-yl)piperidine-4-carbaldehyde

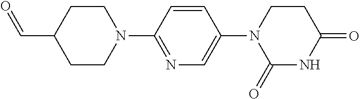 [SMILES: O=CC1CCN(c2ccc(N3CCC(=O)NC3=O)cn2)CC1]

To a solution of 1-(6-(4-(hydroxymethyl)piperidin-1-yl)pyridin-3-yl)dihydropyrimidine-2,4(1H,3H)-dione (160 mg, 0.5 mmol) in DMF (5 mL) was added IBX (191 mg, 0.68 mmol). After stirring at 55° C. for 18 hs, the reaction was quenched with H₂O and extracted with EtOAc (50 mL). The organic phase was separated and washed with NaHCO₃ solution (50 mL) and brine (50 mL). The organic phase was concentrated and used for next step directly.

### Step 2: (R)-5-(tert-butyl)-N-(1-(4-(5-(4-(4-((1-(5-(2,4-dioxotetrahydropyrimidin-1(2H)-yl)pyridin-2-yl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

To a solution of 1-(5-(2,4-dioxotetrahydropyrimidin-1(2H)-yl)pyridin-2-yl)piperidine-4-carbaldehyde (40 mg, 0.13 mmol) in DCM/EtOH (3:1, 20 mL) were added (R)-5-(tert-butyl)-N-(1-(4-(5-(4-(4-chloro-4l4-piperazin-1-yl)phenyl)-1H-pyrazolo[3,4-b]pyridin-3-yl)-2-methylphenyl)ethyl)-1,2,4-oxadiazole-3-carboxamide hydrochloride (80 mg, 0.13 mmol) and NaOAc (33 mg, 0.4 mmol). After stirring for 30 min, NaBH(OAc)₃ (85 mg, 0.4 mmol) was added. The mixture was stirred for 2 hs. The mixture was evaporated and purified by pre-TLC with DCM/MeOH (˜10:1) to give the product (28.34 mg, 25.6%). ¹H NMR (400 MHz, DMSO) δ_{H} 13.78 (s, 1H), 10.35 (s, 1H), 9.49 (d, J=7.2 Hz, 1H), 8.82 (s, 1H), 8.58 (s, 1H), 8.04 (s, 1H), 7.91 (d, J=7.2 Hz, 1H), 7.85 (s, 1H), 7.69 (d, J=7.6 Hz, 2H), 7.60 (d, J=8.0 Hz, 1H), 7.47 (d, J=8.4 Hz, 1H), 7.07 (d, J=8.0 Hz, 2H), 6.84 (d, J=8.8 Hz, 1H), 5.36 (brs, 1H), 4.28 (d, J=11.6 Hz, 2H), 3.70 (brs, 2H), 3.21 (s, 4H), 2.82 (t, J=11.2 Hz, 2H), 2.70 (s, 2H), 2.50 (brs, 6H), 2.21 (s, 2H), 1.88-1.74 (m, 3H), 1.51 (d, J=6.0 Hz, 3H), 1.42 (s, 10H), 1.23-1.09 (brs, 2H); [M+H]⁺=851.8.

### Example 125: 1-(tert-butyl)-N-((1R)-1-(4-(5-(4-(4-(4-(2,6-dioxopiperidin-3-yl)phenethyl)piperazin-1-yl)phenyl)-1H-pyrazolo[3,4-b]pyridin-3-yl)-2-methylphenyl)ethyl)-1H-1,2,3-triazole-4-carboxamide

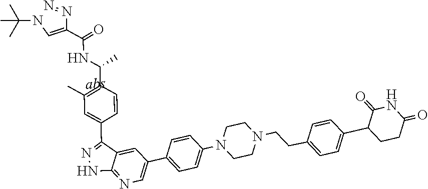

The titled compound was synthesized in the procedures similar to Example 1. ¹H NMR (400 MHz, DMSO) δ_{H} 13.76 (s, 1H), 10.83 (s, 1H), 8.91 (d, J=7.6 Hz, 1H), 8.82 (s, 1H), 8.65 (s, 1H), 8.57 (s, 1H), 7.88 (d, J=7.2 Hz, 1H), 7.82 (s, 1H), 7.69 (d, J=7.6 Hz, 2H), 7.64 (d, J=7.6 Hz, 1H), 7.22 (s, 2H), 7.15 (s, 2H), 7.07 (d, J=6.4 Hz, 2H), 5.38 (s, 1H), 3.81 (s, 1H), 3.22 (s, 4H), 2.78 (s, 2H), 2.63 (s, 8H), 2.50 (s, 3H), 2.17 (brs, 1H), 2.04 (brs, 1H), 1.63 (s, 9H), 1.51 (d, J=6.0 Hz, 3H); [M+H]⁺=779.9.

### Example 127: 5-(tert-butyl)-N-((1R)-1-(4-(5-(5-(5-((1-(4-(2,4-dioxotetrahydropyrimidin-1(2H)-yl)phenyl)piperidin-4-yl)methyl)-2,5-diazabicyclo[2.2.1]heptan-2-yl)pyridin-2-yl)-1H-pyrazolo[3,4-b]pyridin-3-yl)-2-methylphenyl)ethyl)-1,2,4-oxadiazole-3-carboxamide

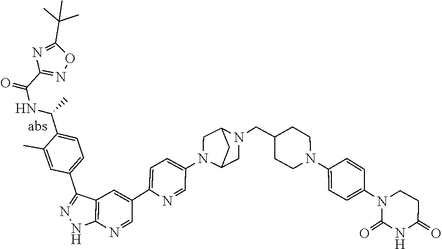 [SMILES: Cc1cc(-c2n[nH]c3ncc(-c4ccc(N5CC6CC5CN6CC5CCN(c6ccc(N7CCC(=O)NC7=O)cc6)CC5)cn4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.79 (s, 1H), 10.25 (s, 1H), 9.48 (s, 1H), 9.21 (s, 1H), 8.89 (s, 1H), 7.87 (s, 1H), 7.84-7.82 (m, 1H), 7.78 (s, 1H), 7.62-7.61 (m, 1H), 7.11 (d, J=8.0 Hz, 3H), 6.93-6.89 (m, 2H), 5.38-5.34 (m, 1H), 4.48-4.46 (m, 1H), 3.68-3.66 (m, 6H), 3.43-3.41 (m, 2H), 3.34-3.33 (m, 5H), 2.98-2.92 (m, 4H), 2.68-2.58 (m, 3H), 2.50-2.45 (m, 2H), 1.84-1.75 (m, 4H), 1.51 (d, J=6.8 Hz, 3H), 1.42 (s, 9H); [M+H]⁺=863.5.

### Example 128: (R)-5-(tert-butyl)-N-(1-(4-(5-(4-(2-(4-(4-(2,4-dioxotetrahydropyrimidin-1(2H)-yl)phenyl)piperazin-1-yl)ethyl)phenyl)-1H-pyrazolo[3,4-b]pyridin-3-yl)-2-methylphenyl)ethyl)-1,2,4-oxadiazole-3-carboxamide

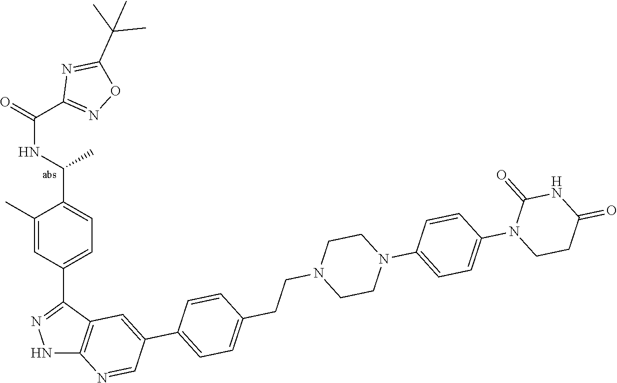 [SMILES: Cc1cc(-c2n[nH]c3ncc(-c4ccc(CCN5CCN(c6ccc(N7CCC(=O)NC7=O)cc6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.87 (s, 1H), 10.31 (s, 1H), 9.98 (s, 1H), 9.50 (d, J=7.8 Hz, 1H), 8.88 (s, 1H), 8.68 (s, 1H), 7.97-7.81 (m, 4H), 7.60 (d, J=7.7 Hz, 1H), 7.45 (d, J=6.9 Hz, 2H), 7.23 (d, J=8.5 Hz, 2H), 7.05 (d, J=8.4 Hz, 2H), 5.36 (t, J=7.3 Hz, 1H), 3.90 (d, J=13.7 Hz, 2H), 3.75-3.65 (m, 4H), 3.47 (s, 2H), 3.30-2.98 (m, 7H), 2.75-2.65 (m, 3H), 1.51 (d, J=6.7 Hz, 3H), 1.42 (s, 9H); [M+H]⁺=781.9.

### Example 129: (R)-5-(tert-butyl)-N-(1-(4-(5-(4-(4-(4-(2,4-dioxotetrahydropyrimidin-1(2H)-yl)phenethyl)piperazin-1-yl)phenyl)-1H-pyrazolo[3,4-b]pyridin-3-yl)-2-methylphenyl)ethyl)-1,2,4-oxadiazole-3-carboxamide

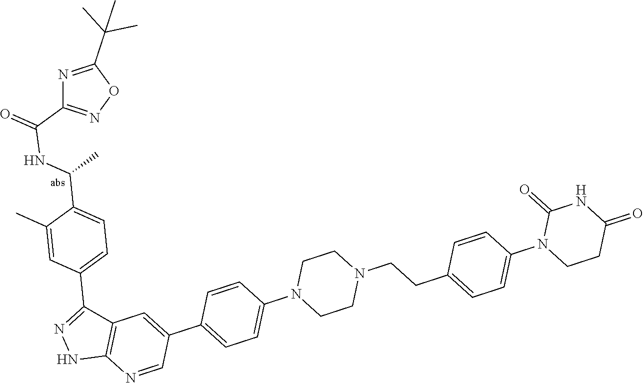 [SMILES: Cc1cc(-c2n[nH]c3ncc(-c4ccc(N5CCN(CCc6ccc(N7CCC(=O)NC7=O)cc6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.78 (s, 1H), 10.35 (s, 1H), 9.59-9.39 (m, 1H), 8.82 (s, 1H), 8.58 (s, 1H), 8.15 (s, 1H), 7.95-7.79 (m, 2H), 7.69 (d, J=8.0 Hz, 2H), 7.60 (d, J=7.6 Hz, 1H), 7.31-7.21 (m, 4H), 7.07 (d, J=7.6 Hz, 2H), 5.42-5.30 (m, 1H), 3.81-3.72 (m, 2H), 3.27-3.18 (m, 5H), 2.83-2.76 (m, 2H), 2.73-2.66 (m, 3H), 2.65-2.58 (m, 6H), 1.51 (d, J=7.2 Hz, 3H), 1.42 (s, 9H); [M+H]⁺=781.8.

### Example 130: 5-(tert-butyl)-N-((1R)-1-(4-(5-(4-(4-(3-(4-((2,6-dioxopiperidin-3-yl)amino)-3-methoxyphenoxy)propyl)piperazin-1-yl)phenyl)-1H-pyrazolo[3,4-b]pyridin-3-yl)-2-methylphenyl)ethyl)-1,2,4-oxadiazole-3-carboxamide

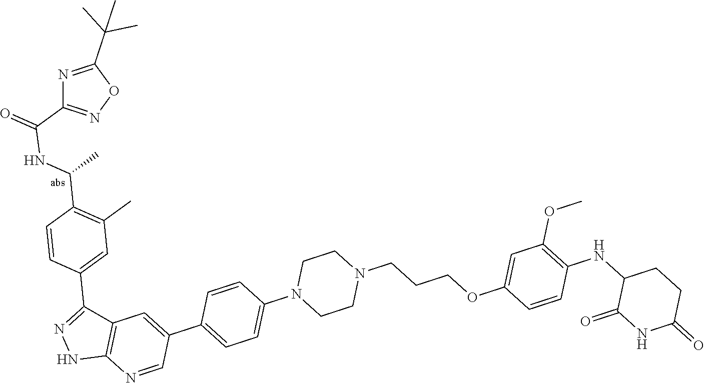 [SMILES: COc1cc(OCCCN2CCN(c3ccc(-c4cnc5[nH]nc(-c6ccc([C@@H](C)NC(=O)c7noc(C(C)(C)C)n7)c(C)c6)c5c4)cc3)CC2)ccc1NC1CCC(=O)NC1=O]

The titled compound was synthesized in the procedures similar to Example 1. ¹H NMR (400 MHz, DMSO) δ_{H} 13.81 (s, 1H), 10.90 (s, 1H), 9.58 (s, 1H), 9.50 (d, J=8.0 Hz, 1H), 8.84 (s, 1H), 8.61 (s, 1H), 7.91 (d, J=8.0 Hz, 1H), 7.85 (s, 1H), 7.77 (d, J=8.0 Hz, 2H), 7.60 (d, J=8.0 Hz, 1H), 7.17 (d, J=8.0 Hz, 2H), 6.60 (d, J=8.0 Hz, 1H), 6.55 (s, 1H), 6.42 (d, J=8.0 Hz, 1H), 5.36 (s, 1H), 4.21 (d, J=12.0 Hz, 1H), 4.05-3.93 (m, 4H), 3.81 (s, 3H), 3.68 (d, J=12.0 Hz, 2H), 3.37 (s, 2H), 3.24 (dd, J=16.0 Hz, 8.0 Hz, 2H), 3.06 (t, J=12.0 Hz, 3H), 2.78 (t, J=12.0 Hz, 1H), 2.58 (d, J=12.0 Hz, 2H), 2.16 (s, 3H), 1.89 (dd, J=16.0 Hz, 8.0 Hz, 1H), 1.51 (d, J=4.0 Hz, 3H), 1.42 (s, 9H); [M+H]⁺=855.9.

### Example 131: 5-(tert-butyl)-N-((1R)-1-(4-(5-(4-(4-(4-((2,6-dioxopiperidin-3-yl)amino)phenethyl)piperazin-1-yl)phenyl)-1H-pyrazolo[3,4-b]pyridin-3-yl)-2-methylphenyl)ethyl)-1,2,4-oxadiazole-3-carboxamide

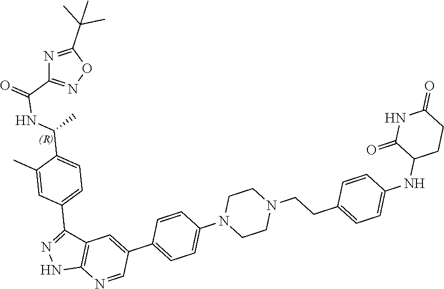

The titled compound was synthesized in the procedures similar to Example 1. ¹H NMR (400 MHz, DMSO) δ_{H} 13.89 (s, 1H), 10.89 (s, 1H), 9.60 (s, 1H), 8.93 (s, 1H), 8.69 (s, 1H), 8.03-8.01 (m, 1H), 7.96 (s, 1H), 7.80 (d, J=8.0 Hz, 2H), 7.71 (d, J=8.0 Hz, 1H), 7.18 (d, J=8.0 Hz, 2H), 7.08 (d, J=8.0 Hz, 2H), 6.72 (d, J=8.0 Hz, 2H), 5.78-5.77 (m, 1H), 5.49-5.43 (m, 1H), 4.38 (s, 1H), 3.32 (s, 4H), 2.85-2.84 (m, 1H), 2.76-2.71 (m, 7H), 2.29-2.17 (m, 2H), 2.07-2.05 (m, 1H), 1.96-1.92 (m, 1H), 1.62 (d, J=8.0 Hz, 3H), 1.53 (s, 9H); [M+H]⁺=795.8.

### Example 132: 5-(tert-butyl)-N-((1R)-1-(4-(5-(5-(5-(4-(2,4-dioxotetrahydropyrimidin-1(2H)-yl)phenethyl)-2,5-diazabicyclo[2.2.1]heptan-2-yl)pyridin-2-yl)-1H-pyrazolo[3,4-b]pyridin-3-yl)-2-methylphenyl)ethyl)-1,2,4-oxadiazole-3-carboxamide

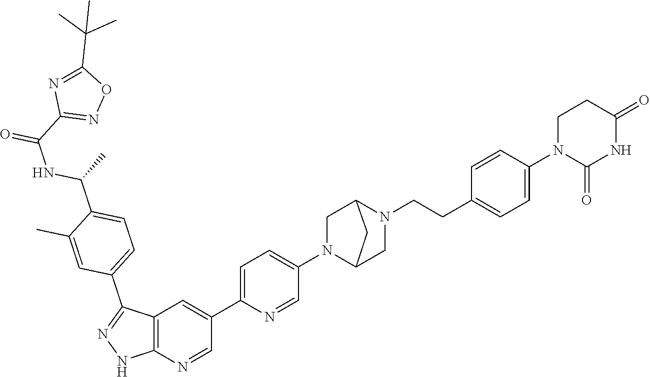 [SMILES: Cc1cc(-c2n[nH]c3ncc(-c4ccc(N5CC6CC5CN6CCc5ccc(N6CCC(=O)NC6=O)cc5)cn4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.79 (s, 1H), 10.33 (s, 1H), 9.50 (d, J=4.0 Hz, 1H), 9.21 (s, 1H), 8.89 (s, 1H), 8.14 (s, 1H), 8.02-7.95 (m, 1H), 7.93-7.82 (m, 1H), 7.82 (s, 1H), 7.66-7.62 (m, 1H), 7.26-7.22 (m, 4H), 7.12-7.08 (m, 1H), 5.38-5.34 (m, 1H), 4.50 (s, 1H), 3.45-3.35 (m, 3H), 3.34-3.32 (m, 3H), 3.01-2.98 (m, 2H), 2.70-2.64 (m, 4H), 2.56 (s, 3H), 1.85-1.82 (m, 2H), 1.80-1.78 (m, 1H), 1.52 (d, J=4.0 Hz, 3H), 1.42 (s, 9H); [M+H]⁺=794.7.

### Example 133: 5-(tert-butyl)-N-((1R)-1-(4-(5-(5-(5-(4-(2,6-dioxopiperidin-3-yl)phenethyl)-2,5-diazabicyclo[2.2.1]heptan-2-yl)pyridin-2-yl)-1H-pyrazolo[3,4-b]pyridin-3-yl)-2-methylphenyl)ethyl)-1,2,4-oxadiazole-3-carboxamide

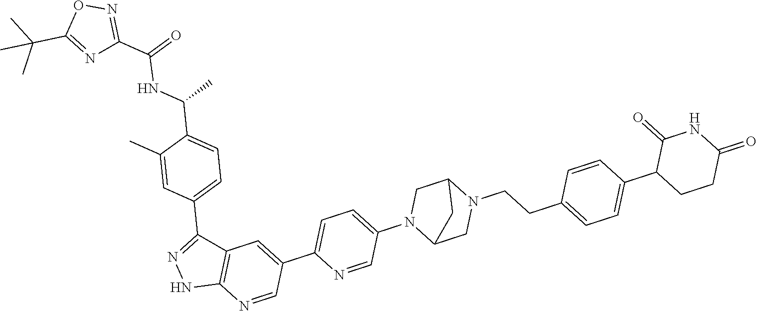 [SMILES: Cc1cc(-c2n[nH]c3ncc(-c4ccc(N5CC6CC5CN6CCc5ccc(C6CCC(=O)NC6=O)cc5)cn4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.79 (s, 1H), 10.81 (s, 1H), 9.49 (d, J=4.0 Hz, 1H), 9.21 (s, 1H), 8.89 (s, 1H), 8.18-8.16 (m, 1H), 8.02-7.95 (m, 1H), 7.93-7.83 (m, 1H), 7.83 (s, 1H), 7.64-7.60 (m, 1H), 7.18-7.14 (m, 2H), 7.14-7.10 (m, 3H), 5.38-5.34 (m, 1H), 4.50 (s, 1H), 3.79-3.76 (m, 1H), 3.68 (s, 1H), 3.35-3.32 (m, 2H), 2.98-2.96 (m, 1H), 2.85-2.70 (m, 5H), 2.70-2.52 (m, 2H), 2.50 (s, 3H), 2.08-2.01 (m, 1H), 2.01-1.89 (m, 1H), 1.85-1.82 (m, 1H), 1.82-1.80 (m, 1H), 1.52 (d, J=4.0 Hz, 3H), 1.42 (s, 9H); [M+H]⁺=793.8.

### Example 134: (R)-5-(tert-butyl)-N-(1-(4-(5-(4-(4-((1-(4-(2,4-dioxotetrahydropyrimidin-1(2H)-yl)-2-fluorophenyl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

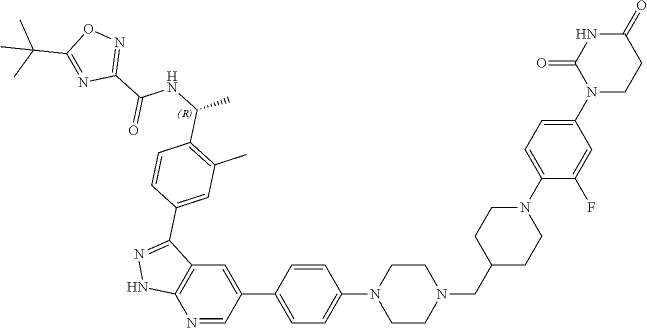

The titled compound was synthesized in the procedures similar to Example 1. ¹H NMR (400 MHz, DMSO) δ_{H} 13.77 (s, 1H), 10.37 (s, 1H), 9.49 (d, J=4.0 Hz, 1H), 8.82 (s, 1H), 8.58 (s, 1H), 7.92 (d, J=8.0 Hz, 1H), 7.85 (s, 1H), 7.69 (d, J=8.0 Hz, 2H), 7.60 (d, J=8.0 Hz, 1H), 7.17 (d, J=12.0 Hz, 1H), 7.10-7.00 (m, 4H), 5.36 (s, 1H), 3.74 (s, 2H), 3.36 (s, 1H), 3.22 (s, 4H), 3.08-2.82 (m, 2H), 2.72-2.64 (m, 4H), 2.58-2.52 (m, 7H), 2.26 (s, 2H), 1.84 (d, J=8.0 Hz, 2H), 1.71 (s, 1H), 1.50 (d, J=8.0 Hz, 3H), 1.42 (s, 9H), 1.36-1.26 (m, 2H); [M+H]⁺=868.8.

### Example 135: (R)-5-(tert-butyl)-N-(1-(4-(5-(4-(4-((1-(4-(2,4-dioxotetrahydropyrimidin-1(2H)-yl)-2-methylphenyl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

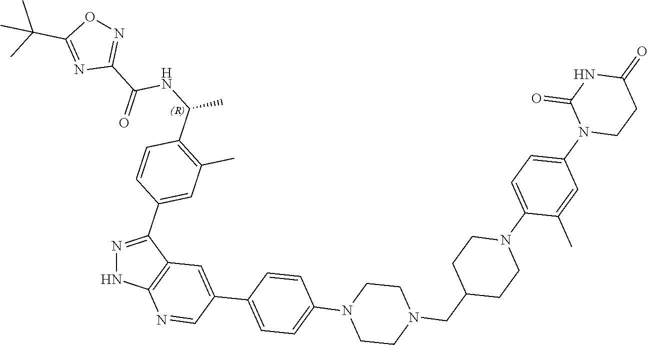

The titled compound was synthesized in the procedures similar to Example 1. ¹H NMR (400 MHz, DMSO) δ_{H} 13.77 (s, 1H), 10.37 (s, 1H), 9.49 (d, J=4.0 Hz, 1H), 8.82 (s, 1H), 8.58 (s, 1H), 7.92 (d, J=8.0 Hz, 1H), 7.85 (s, 1H), 7.69 (d, J=8.0 Hz, 2H), 7.60 (d, J=8.0 Hz, 1H), 7.17 (d, J=12.0 Hz, 1H), 7.10-7.00 (m, 4H), 5.36 (s, 1H), 3.74 (s, 2H), 3.36 (s, 1H), 3.22 (s, 4H), 3.08-2.82 (m, 2H), 2.72-2.64 (m, 4H), 2.58-2.52 (m, 7H), 2.26 (s, 2H), 2.24 (s, 3H), 1.84 (d, J=8.0 Hz, 2H), 1.71 (s, 1H), 1.50 (d, J=8.0 Hz, 3H), 1.42 (s, 9H), 1.36-1.26 (m, 2H); [M+H]⁺=864.8.

### Example 136: 5-(tert-butyl)-N-((1R)-1-(4-(5-(4-(4-(2-(1-(5-(2,6-dioxopiperidin-3-yl)-6-methylpyridin-2-yl)piperidin-4-yl)ethyl)piperazin-1-yl)phenyl)-1H-pyrazolo[3,4-b]pyridin-3-yl)-2-methylphenyl)ethyl)-1,2,4-oxadiazole-3-carboxamide

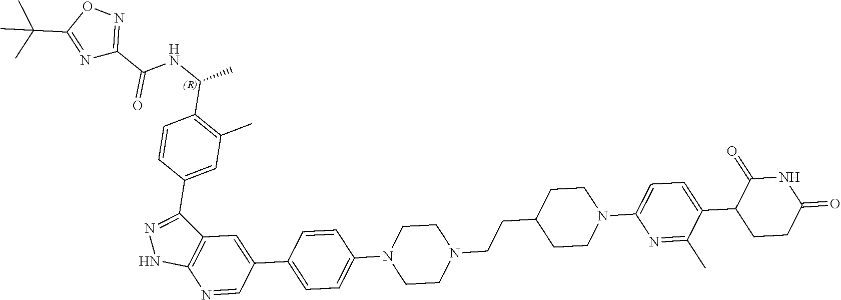

The titled compound was synthesized in the procedures similar to Example 1. ¹H NMR (400 MHz, DMSO) δ_{H} 13.77 (s, 1H), 10.80 (s, 1H), 9.49 (d, J=4.0 Hz, 1H), 8.82 (s, 1H), 8.58 (s, 1H), 7.90 (d, J=8.0 Hz, 1H), 7.85 (s, 1H), 7.69 (d, J=8.0 Hz, 2H), 7.61 (d, J=8.0 Hz, 1H), 7.22 (d, J=8.0 Hz, 1H), 7.06 (d, J=8.0 Hz, 2H), 6.60 (d, J=8.0 Hz, 1H), 5.36 (s, 1H), 4.26 (d, J=8.0 Hz, 2H), 3.90 (d, J=8.0 Hz, 1H), 3.20 (s, 4H), 3.08-2.82 (m, 2H), 2.79-2.68 (m, 3H), 2.53 (s, 5H), 2.39 (s, 3H), 2.29 (s, 3H), 2.20-2.06 (m, 1H), 1.95-1.89 (m, 1H), 1.75 (d, J=12.0 Hz, 2H), 1.52 (d, J=8.0 Hz, 3H), 1.48-1.38 (m, 11H), 1.19-1.08 (m, 2H); [M+H]⁺=878.6.

### Example 137: 5-(tert-butyl)-N-((1R)-1-(4-(5-(4-(4-(4-(2,6-dioxopiperidin-3-yl)-3-methoxyphenethyl)piperazin-1-yl)phenyl)-1H-pyrazolo[3,4-b]pyridin-3-yl)-2-methylphenyl)ethyl)-1,2,4-oxadiazole-3-carboxamide

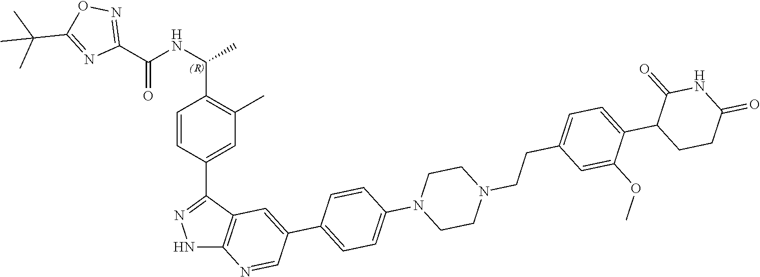

The titled compound was synthesized in the procedures similar to Example 1. ¹H NMR (400 MHz, DMSO) δ_{H} 13.75 (s, 1H), 10.71 (s, 1H), 9.47 (d, J=8.0 Hz, 1H), 8.80 (s, 1H), 8.56 (s, 1H), 7.88 (d, J=8.0 Hz, 1H), 7.83 (s, 1H), 7.66 (d, J=8.0 Hz, 2H), 7.58 (d, J=8.0 Hz, 1H), 7.10-6.98 (m, 3H), 6.91 (s, 1H), 6.79 (d, J=8.0 Hz, 1H), 5.34 (s, 1H), 3.85 (d, J=8.0 Hz, 1H), 3.72 (s, 3H), 3.21 (s, 4H), 3.06-2.83 (m, 2H), 2.76 (s, 2H), 2.68-2.56 (m, 7H), 2.43-2.37 (m, 1H), 2.16 (dd, J=16.0 Hz, 4.0 Hz, 1H), 1.89 (s, 1H), 1.49 (d, J=4.0 Hz, 3H), 1.40 (s, 9H); [M+H]⁺=810.8.

### Example 138: (R)-5-(tert-butyl)-N-(1-(4-(5-(4-(4-((1-(3-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

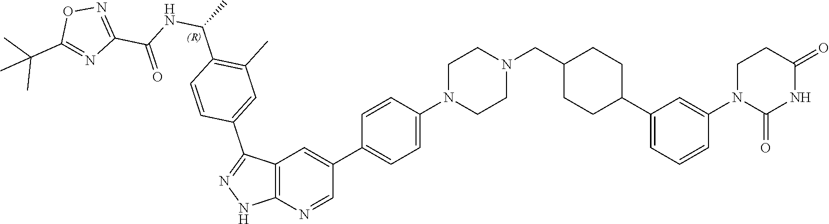

The titled compound was synthesized in the procedures similar to Example 1. ¹H NMR (400 MHz, DMSO) δ_{H} 13.77 (s, 1H), 10.30 (s, 1H), 9.48 (d, J=8.0 Hz, 1H), 8.82 (s, 1H), 8.58 (s, 1H), 7.90 (d, J=8.0 Hz, 1H), 7.85 (s, 1H), 7.69 (d, J=8.0 Hz, 2H), 7.60 (d, J=8.0 Hz, 1H), 7.18 (t, J=8.0 Hz, 1H), 7.07 (d, J=8.0 Hz, 2H), 6.88 (s, 1H), 6.82 (d, J=8.0 Hz, 1H), 6.68 (d, J=8.0 Hz, 1H), 5.36 (s, 1H), 3.80-3.65 (m, 4H), 3.21 (s, 4H), 3.08-2.82 (m, 1H), 2.68 (s, 4H), 2.52 (s, 6H), 2.23 (s, 2H), 1.82 (d, J=12.0 Hz, 2H), 1.73 (s, 1H), 1.51 (d, J=8.0 Hz, 3H), 1.42 (s, 9H), 1.26-1.16 (m, 2H); [M+H]⁺=850.8.

### Example 139 and 140: 5-(tert-butyl)-N—((R)-1-(4-(5-(4-(4-(2-(5-((S)-2,6-dioxopiperidin-3-yl)pyridin-2-yl)ethyl)piperazin-1-yl)phenyl)-1H-pyrazolo[3,4-b]pyridin-3-yl)-2-methylphenyl)ethyl)-1,2,4-oxadiazole-3-carboxamide and 5-(tert-butyl)-N—((R)-1-(4-(5-(4-(4-(2-(5-((R)-2,6-dioxopiperidin-3-yl)pyridin-2-yl)ethyl)piperazin-1-yl)phenyl)-1H-pyrazolo[3,4-b]pyridin-3-yl)-2-methylphenyl)ethyl)-1,2,4-oxadiazole-3-carboxamide

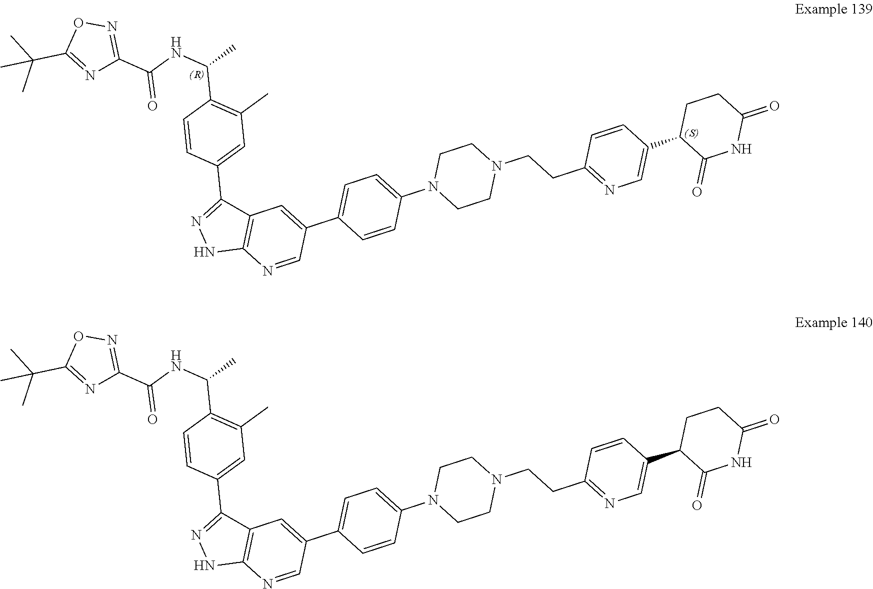

A racemic compound of 5-(tert-butyl)-N-((1R)-1-(4-(5-(4-(4-(2-(5-(2,6-dioxopiperidin-3-yl)pyridin-2-yl)ethyl)piperazin-1-yl)phenyl)-1H-pyrazolo[3,4-b]pyridin-3-yl)-2-methylphenyl)ethyl)-1,2,4-oxadiazole-3-carboxamide (92.1 mg) was separated by PREP_CHIRAL_HPLC with following conditions: Column: CHIRALPAK IA, 2*25 cm, 5 μm; Mobile Phase A: Hex:DCM=3:1 (0.5% 2M NH3-MeOH)—HPLC, Mobile Phase B: IPA; Flow rate: 17 mL/min; Gradient: 50% B to 50% B in 26 min; Detector: 220/254 nm; RT1: 12.255 min; RT2: 17.433 min; Sample Solvent: EtOH:DCM=3:1; Injection Volume: 1 mL; Number Of Runs: 3. This resulted in Example 139 (29.8 mg, the first isomer) and Example 140 (26.6 mg, the second isomer). Example 139: ¹H NMR (400 MHz, DMSO) δ_{H} 13.77 (s, 1H), 10.91 (s, 1H), 9.47 (d, J=8.0 Hz, 1H), 8.82 (s, 1H), 8.58 (s, 1H), 8.36 (s, 1H), 7.91 (d, J=8.0 Hz, 1H), 7.85 (s, 1H), 7.69 (d, J=8.0 Hz, 2H), 7.62-7.58 (m, 2H), 7.31 (d, J=8.0 Hz, 1H), 7.08 (d, J=8.0 Hz, 2H), 5.41-5.30 (m, 1H), 3.84-3.95 (m, 1H), 3.31-3.15 (m, 4H), 3.02-2.85 (m, 2H), 2.85-2.52 (m, 8H), 2.48 (s, 3H), 2.39-2.20 (m, 1H), 2.11-1.98 (m, 1H), 1.51 (d, J=8.0 Hz, 3H), 1.43 (s, 9H); [M+H]⁺=781.4. Example 140: ¹H NMR (400 MHz, DMSO) δ_{H} 13.77 (s, 1H), 10.90 (s, 1H), 9.47 (d, J=8.0 Hz, 1H), 8.82 (s, 1H), 8.58 (s, 1H), 8.36 (s, 1H), 7.91 (d, J=8.0 Hz, 1H), 7.85 (s, 1H), 7.69 (d, J=8.0 Hz, 2H), 7.65-7.57 (m, 2H), 7.31 (d, J=8.0 Hz, 1H), 7.08 (d, J=8.0 Hz, 2H), 5.42-5.30 (m, 1H), 4.97-4.88 (m, 1H), 3.35-3.20 (m, 4H), 3.15-2.90 (m, 2H), 2.91-2.51 (m, 8H), 2.49 (s, 3H), 2.40-2.21 (m, 1H), 2.11-2.00 (m, 1H), 1.51 (d, J=8.0 Hz, 3H), 1.45 (s, 9H); [M+H]⁺=781.4.

### Example 141: 5-(tert-butyl)-N-((1R)-1-(4-(5-(4-(4-(2-(5-(2,6-dioxopiperidin-3-yl)-4-methylpyridin-2-yl)ethyl)piperazin-1-yl)phenyl)-1H-pyrazolo[3,4-b]pyridin-3-yl)-2-methylphenyl)ethyl)-1,2,4-oxadiazole-3-carboxamide

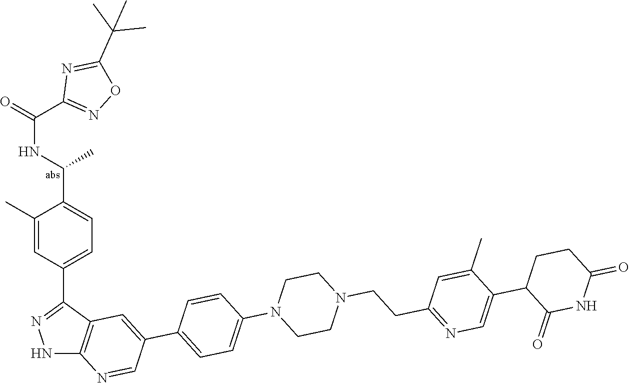 [SMILES: Cc1cc(CCN2CCN(c3ccc(-c4cnc5[nH]nc(-c6ccc([C@@H](C)NC(=O)c7noc(C(C)(C)C)n7)c(C)c6)c5c4)cc3)CC2)ncc1C1CCC(=O)NC1=O]

The titled compound was synthesized in the procedures similar to Example 1. ¹H NMR (400 MHz, DMSO) δ_{H} 13.79 (s, 1H), 10.91 (s, 1H), 9.48 (s, 1H), 8.83 (s, 1H), 8.59 (s, 1H), 8.25 (s, 1H), 7.98-7.54 (m, 5H), 7.25-7.06 (m, 3H), 5.36 (s, 1H), 4.17-4.02 (m, 1H), 3.60 (s, 6H), 3.14 (s, 1H), 2.87-2.52 (m, 10H), 2.37-2.24 (m, 4H), 1.99 (s, 1H), 1.51 (s, 3H), 1.42 (s, 9H); [M+H]⁺=795.8.

### Example 142:5-(tert-butyl)-N-((1R)-1-(4-(5-(4-(4-(3-(4-((2,6-dioxopiperidin-3-yl)amino)-3-fluorophenoxy)propyl)piperazin-1-yl)phenyl)-1H-pyrazolo[3,4-b]pyridin-3-yl)-2-methylphenyl)ethyl)-1,2,4-oxadiazole-3-carboxamide

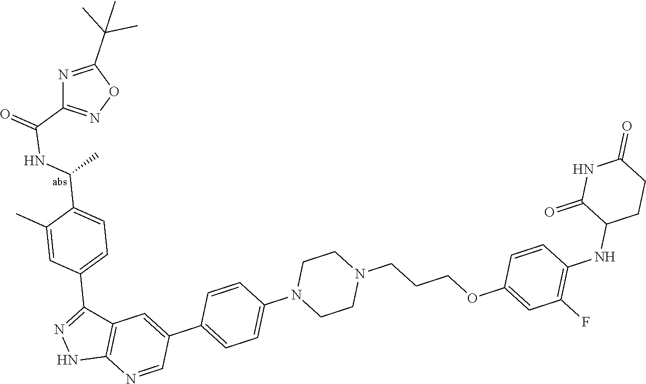 [SMILES: Cc1cc(-c2n[nH]c3ncc(-c4ccc(N5CCN(CCCOc6ccc(NC7CCC(=O)NC7=O)c(F)c6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.78 (s, 1H), 10.81 (s, 1H), 9.49 (d, J=7.2 Hz, 1H), 8.82 (s, 1H), 8.58 (s, 1H), 8.35 (s, 1H), 7.95-7.81 (m, 2H), 7.74-7.58 (m, 3H), 7.07 (d, J=7.4 Hz, 2H), 6.83-6.73 (m, 2H), 6.62 (d, J=8.6 Hz, 1H), 5.43-5.31 (m, 1H), 5.20-5.11 (m, 1H), 4.29 (s, 1H), 3.95 (s, 2H), 3.22 (s, 5H), 2.82-2.66 (m, 1H), 2.61-2.52 (m, 7H), 2.15-1.80 (m, 5H), 1.51 (d, J=5.8 Hz, 3H), 1.42 (s, 9H); [M+H]⁺=843.9.

### Example 143: (R)-5-(tert-butyl)-N-(1-(4-(5-(4-(4-(2-(4-(2,4-dioxotetrahydropyrimidin-1(2H)-yl)-1H-pyrazol-1-yl)ethyl)piperazin-1-yl)phenyl)-1H-pyrazolo[3,4-b]pyridin-3-yl)-2-methylphenyl)ethyl)-1,2,4-oxadiazole-3-carboxamide

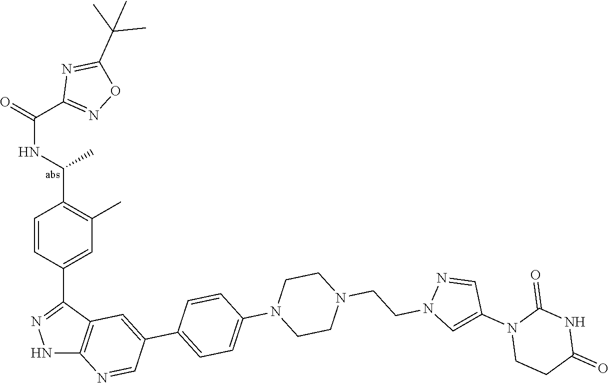 [SMILES: Cc1cc(-c2n[nH]c3ncc(-c4ccc(N5CCN(CCn6cc(N7CCC(=O)NC7=O)cn6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.81 (s, 1H), 11.22 (s, 1H), 10.44 (s, 1H), 9.49 (d, J=7.2 Hz, 1H), 8.84 (s, 1H), 8.60 (s, 1H), 8.10 (s, 1H), 7.92 (d, J=6.8 Hz, 1H), 7.85 (s, 1H), 7.79-7.69 (m, 3H), 7.63-7.58 (m, 1H), 7.14 (d, J=7.2 Hz, 2H), 5.40-5.30 (m, 1H), 4.70-4.60 (m, 2H), 4.00-3.85 (m, 2H), 3.84-3.75 (m, 2H), 3.70-3.52 (m, 4H), 3.28-3.12 (m, 4H), 2.75-2.65 (m, 2H), 1.51 (d, J=6.0 Hz, 3H), 1.42 (s, 9H); [M+H]⁺=771.8.

### Example 144: (R)-5-(tert-butyl)-N-(1-(4-(5-(4-(4-((4-(4-(2,4-dioxotetrahydropyrimidin-1(2H)-yl)phenyl)piperazin-1-yl)methyl)piperidin-1-yl)phenyl)-1H-pyrazolo[3,4-b]pyridin-3-yl)-2-methylphenyl)ethyl)-1,2,4-oxadiazole-3-carboxamide

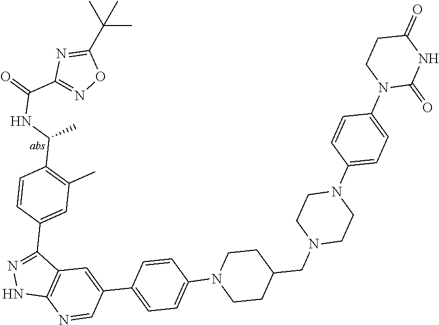 [SMILES: Cc1cc(-c2n[nH]c3ncc(-c4ccc(N5CCC(CN6CCN(c7ccc(N8CCC(=O)NC8=O)cc7)CC6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.77 (s, 1H), 10.27 (s, 1H), 9.48 (d, J=7.2 Hz, 1H), 8.81 (s, 1H), 8.57 (s, 1H), 7.91 (d, J=7.6 Hz, 1H), 7.85 (s, 1H), 7.67 (d, J=8.0 Hz, 2H), 7.60 (d, J=8.0 Hz, 1H), 7.16 (d, J=8.0 Hz, 2H), 7.06 (d, J=7.6 Hz, 2H), 6.94 (d, J=8.0 Hz, 2H), 5.36 (brs, 1H), 3.79 (d, J=10.8 Hz, 2H), 3.71 (t, J=6.4 Hz, 2H), 3.14 (s, 4H), 2.76-2.68 (m, 4H), 2.50 (brs, 6H), 2.23 (brs, 2H), 1.87-1.69 (m, 3H), 1.51 (d, J=6.4 Hz, 3H), 1.42 (s, 9H), 1.24 (brs, 3H); [M+H]⁺=850.8.

### Example 145: 5-(tert-butyl)-N-((1R)-1-(4-(5-(5-(1-(2-(5-(2,6-dioxopiperidin-3-yl)-4-methylpyridin-2-yl)ethyl)piperidin-4-yl)pyridin-2-yl)-1H-pyrazolo[3,4-b]pyridin-3-yl)-2-methylphenyl)ethyl)-1,2,4-oxadiazole-3-carboxamide

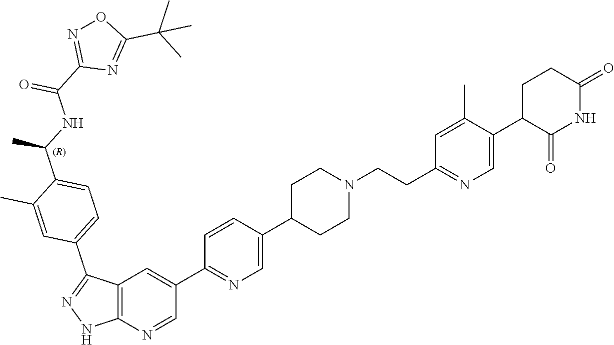

The titled compound was synthesized in the procedures similar to Example 1. ¹H NMR (400 MHz, DMSO) δ_{H} 13.92 (s, 1H), 10.92 (s, 1H), 9.50 (d, J=8.0 Hz, 1H), 9.29 (s, 1H), 9.04 (s, 1H), 8.66 (s, 1H), 8.27 (s, 1H), 8.19-8.14 (m, 1H), 7.90 (d, J=8.0 Hz, 1H), 7.84-7.82 (m, 2H), 7.63 (d, J=8.0 Hz, 1H), 7.20 (s, 1H), 5.37 (s, 1H), 4.13-4.10 (m, 1H), 3.54 (s, 2H), 3.12 (s, 2H), 2.91-2.75 (m, 3H), 2.58-2.55 (m, 7H), 2.33-2.27 (m, 4H), 2.02-2.00 (m, 5H), 1.52 (d, J=8.0 Hz, 3H), 1.43 (s, 9H); [M+H]⁺=795.6.

### Example 146: 5-(tert-butyl)-N-((1R)-1-(4-(5-(5-(4-(4-(2,6-dioxopiperidin-3-yl)-3-fluorophenethyl)piperazin-1-yl)pyridin-2-yl)-1H-pyrazolo[3,4-b]pyridin-3-yl)-2-methylphenyl)ethyl)-1,2,4-oxadiazole-3-carboxamide

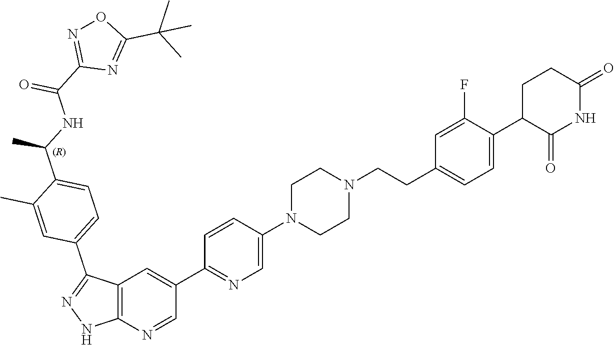

The titled compound was synthesized in the procedures similar to Example 1. ¹H NMR (400 MHz, DMSO) δ_{H} 13.82 (s, 1H), 10.87 (s, 1H), 9.48 (d, J=8.0 Hz, 1H), 9.23 (s, 1H), 8.93 (s, 1H), 8.45 (s, 1H), 8.03 (d, J=8.0 Hz, 1H), 7.90-7.84 (m, 2H), 7.63 (d, J=8.0 Hz, 1H), 7.46 (d, J=8.0 Hz, 1H), 7.24-7.20 (m, 1H), 7.14-7.07 (m, 2H), 5.37 (s, 1H), 4.02-4.00 (m, 1H), 3.29 (s, 4H), 2.80-2.71 (m, 3H), 2.71-2.50 (m, 10H), 2.23-2.17 (m, 1H), 2.01-1.97 (m, 1H), 1.52 (d, J=8.0 Hz, 3H), 1.43 (s, 9H); [M+H]⁺=799.8.

### Example 147: 5-(tert-butyl)-N-((1R)-1-(4-(5-(5-(4-(2-(5-(2,6-dioxopiperidin-3-yl)-4-methylpyridin-2-yl)ethyl)piperazin-1-yl)pyridin-2-yl)-1H-pyrazolo[3,4-b]pyridin-3-yl)-2-methylphenyl)ethyl)-1,2,4-oxadiazole-3-carboxamide

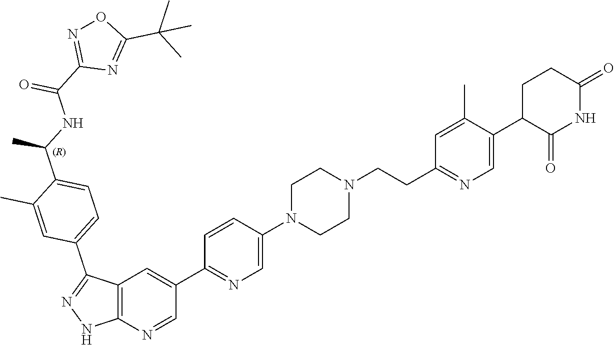

The titled compound was synthesized in the procedures similar to Example 1. ¹H NMR (400 MHz, DMSO) δ_{H} 13.82 (s, 1H), 10.90 (s, 1H), 9.48 (d, J=8.0 Hz, 1H), 9.23 (s, 1H), 8.93 (s, 1H), 8.45 (s, 1H), 8.21 (s, 1H), 8.04 (d, J=8.0 Hz, 1H), 7.90-7.83 (m, 2H), 7.62 (d, J=8.0 Hz, 1H), 7.45 (d, J=8.0 Hz, 1H), 7.15 (s, 1H), 5.37 (s, 1H), 4.08-4.06 (m, 1H), 3.26 (s, 4H), 2.89-2.84 (m, 2H), 2.79-2.54 (m, 11H), 2.33-2.24 (m, 4H), 1.99-1.97 (m, 1H), 1.52 (d, J=8.0 Hz, 3H), 1.43 (s, 9H); [M+H]⁺=796.7.

### Example 148: 5-(tert-butyl)-N-((1R)-1-(4-(5-(4-(4-(2-(5-(2,6-dioxopiperidin-3-yl)-4-methylpyridin-2-yl)ethyl)piperazin-1-yl)phenyl)-1H-pyrazolo[3,4-b]pyridin-3-yl)-3-fluoro-2-methylphenyl)ethyl)-1,2,4-oxadiazole-3-carboxamide

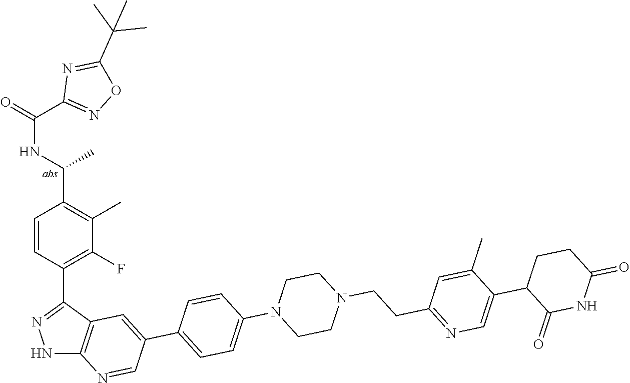 [SMILES: Cc1cc(CCN2CCN(c3ccc(-c4cnc5[nH]nc(-c6ccc([C@@H](C)NC(=O)c7noc(C(C)(C)C)n7)c(C)c6F)c5c4)cc3)CC2)ncc1C1CCC(=O)NC1=O]

The titled compound was synthesized in the procedures similar to Example 1. ¹H NMR (400 MHz, DMSO) δ_{H} 13.95 (s, 1H), 10.90 (s, 1H), 9.57 (d, J=8.0 Hz, 1H), 8.84 (s, 1H), 8.31 (s, 1H), 8.21 (s, 1H), 7.71 (t, J=8.0 Hz, 1H), 7.63 (d, J=8.0 Hz, 2H), 7.46 (d, J=8.0 Hz, 1H), 7.15 (s, 1H), 7.06 (d, J=8.0 Hz, 1H), 5.38 (s, 1H), 4.06 (d, J=8.0 Hz, 1H), 3.22 (s, 4H), 2.88 (s, 3H), 2.75-2.69 (m, 3H), 2.62 (s, 3H), 2.56 (s, 1H), 2.40 (s, 3H), 2.29-2.26 (m, 1H), 2.24-2.19 (m, 3H), 2.00-1.94 (m, 1H), 1.52 (d, J=6.7 Hz, 1H), 1.43 (s, 3H); [M+H]⁺=813.5.

### Example 149: 5-(tert-butyl)-N-((1R)-1-(4-(5-(4-(4-(2-(5-(2,6-dioxopiperidin-3-yl)-4-methylpyridin-2-yl)ethyl)piperazin-1-yl)phenyl)-1H-pyrazolo[3,4-b]pyridin-3-yl)-2-(hydroxymethyl)phenyl)ethyl)-1,2,4-oxadiazole-3-carboxamide

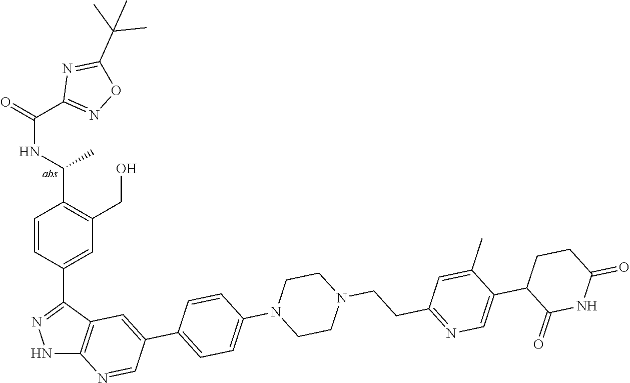 [SMILES: Cc1cc(CCN2CCN(c3ccc(-c4cnc5[nH]nc(-c6ccc([C@@H](C)NC(=O)c7noc(C(C)(C)C)n7)c(CO)c6)c5c4)cc3)CC2)ncc1C1CCC(=O)NC1=O]

The titled compound was synthesized in the procedures similar to Example 1. ¹H NMR (400 MHz, DMSO) δ_{H} 13.79 (s, 1H), 10.90 (s, 1H), 9.52 (d, J=8.0 Hz, 1H), 8.83 (s, 1H), 8.59 (s, 1H), 8.23 (s, 1H), 8.06 (s, 1H), 7.97 (d, J=8.0 Hz, 1H), 7.68 (t, J=8.0 Hz, 3H), 7.25-7.05 (m, 3H), 5.45-5.25 (m, 2H), 4.90-4.70 (m, 2H), 4.10 (d, J=8.0 Hz, 1H), 3.30-3.18 (m, 4H), 3.05-2.86 (m, 3H), 2.73-2.67 (m, 3H), 2.55 (s, 2H), 2.32-2.20 (m, 5H), 2.00 (s, 1H), 1.91 (s, 1H), 1.53 (d, J=8.0 Hz, 3H), 1.42 (s, 9H); [M+H]⁺=811.7.

### Example 150 and 151: (R)-1-(tert-butyl)-N-(4-(5-(4-(4-(4-(2,6-dioxopiperidin-3-yl)phenethyl)piperazin-1-yl)phenyl)-1H-pyrazolo[3,4-b]pyridin-3-yl)-2-methylbenzyl)-1H-1,2,3-triazole-4-carboxamide and (S)-1-(tert-butyl)-N-(4-(5-(4-(4-(4-(2,6-dioxopiperidin-3-yl)phenethyl)piperazin-1-yl)phenyl)-1H-pyrazolo[3,4-b]pyridin-3-yl)-2-methylbenzyl)-1H-1,2,3-triazole-4-carboxamide

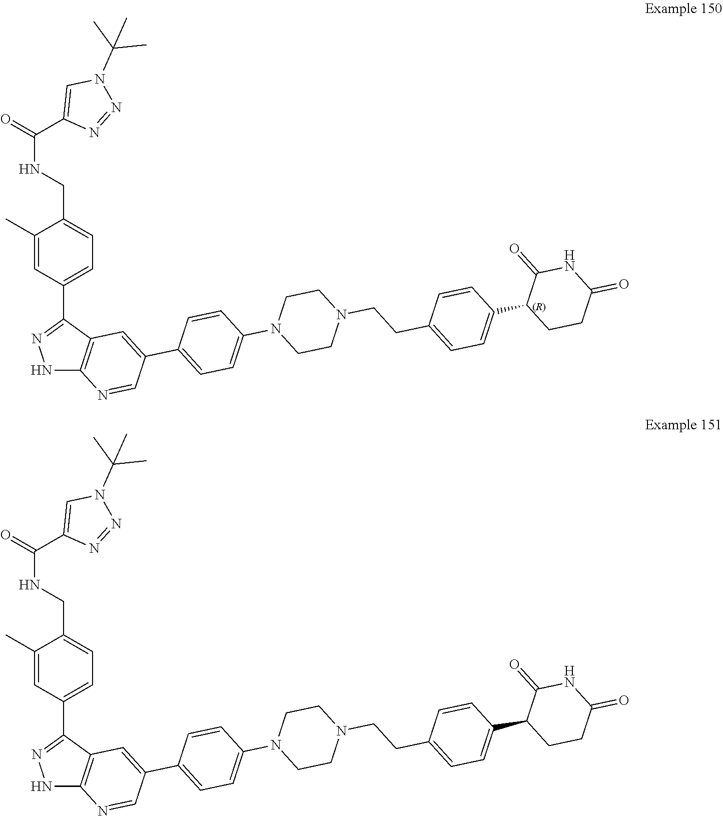

Each enantiomer was separated from Example 108 by using preparative HPLC on a CHIRALPAK IA-3 with DCM:EtOH=40:60 as an eluent. The enantiomeric excesses were determined by using HPLC on a CHIRALPAK IA-3 with DCM:EtOH=40:60 as an eluent at a flow rate of 1.0 mL/min. The first enantiomer Example 150 was eluted at the retention time of 1.441 min, and the other enantiomer Example 151 was eluted at the retention time of 1.946 min. Example 150: ¹H NMR (400 MHz, CDCl₃) δ_{H} 10.55 (s, 1H), 8.80 (s, 1H), 8.41 (s, 1H), 8.18 (s, 1H), 7.91-7.78 (m, 3H), 7.57-7.40 (m, 4H), 7.26-7.15 (m, 2H), 7.76 (d, J=8.8 Hz, 2H), 4.72 (d, J=5.6 Hz, 2H), 3.79-3.76 (m, 2H), 3.33 (brs, 4H), 2.86-2.66 (m, 10H), 2.49 (s, 3H), 2.29-2.23 (m, 2H), 1.71 (s, 9H); [M+H]⁺=765.55. Example 151: 10.55 (s, 1H), 8.81 (s, 1H), 8.42 (s, 1H), 8.20 (s, 1H), 7.91-7.79 (m, 3H), 7.58-7.42 (m, 4H), 7.27-7.15 (m, 2H), 7.76 (d, J=8.8 Hz, 2H), 4.74 (d, J=5.6 Hz, 2H), 3.79-3.76 (m, 2H), 3.33 (brs, 4H), 2.86-2.66 (m, 10H), 2.49 (s, 3H), 2.29-2.23 (m, 2H), 1.75 (s, 9H); [M+H]⁺=765.55.

### Example 152 and 153: 5-(tert-butyl)-N—((R)-1-(4-(5-(6-(1-(4-((R)-2,6-dioxopiperidin-3-yl)phenethyl)piperidin-4-yl)pyridin-3-yl)-1H-pyrazolo[3,4-b]pyridin-3-yl)-2-methylphenyl)ethyl)-1,2,4-oxadiazole-3-carboxamide and 5-(tert-butyl)-N—((R)-1-(4-(5-(6-(1-(4-((S)-2,6-dioxopiperidin-3-yl)phenethyl)piperidin-4-yl)pyridin-3-yl)-1H-pyrazolo[3,4-b]pyridin-3-yl)-2-methylphenyl)ethyl)-1,2,4-oxadiazole-3-carboxamide

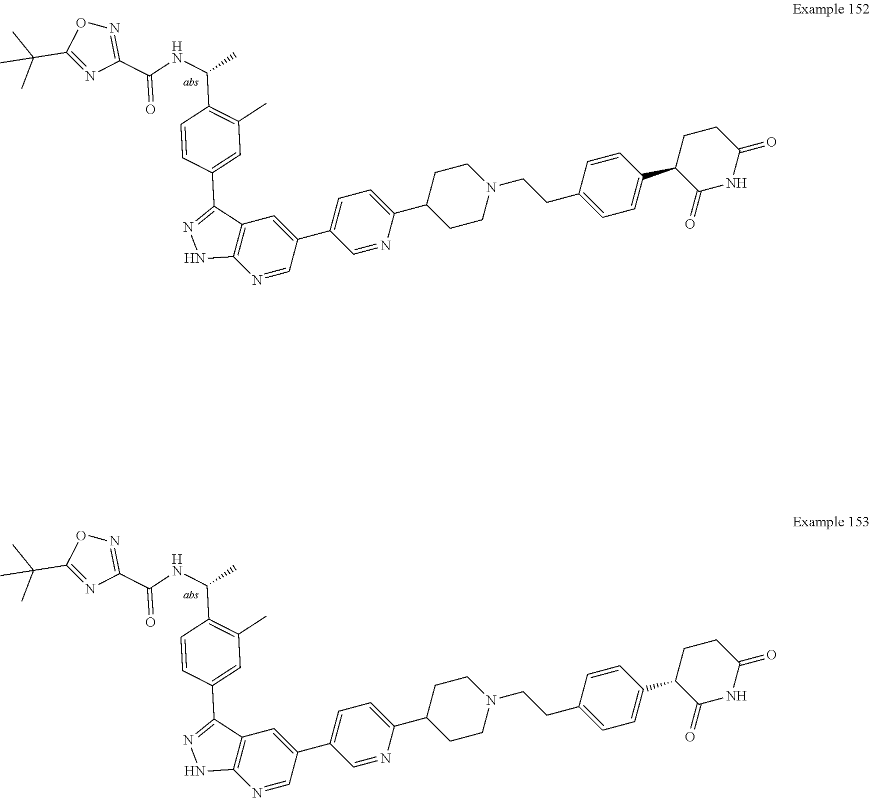 [SMILES: Cc1cc(-c2n[nH]c3ncc(-c4ccc(C5CCN(CCc6ccc([C@H]7CCC(=O)NC7=O)cc6)CC5)nc4)cc23)ccc1[C@@H](C)NC(=O)c1noc(C(C)(C)C)n1 ; Cc1cc(-c2n[nH]c3ncc(-c4ccc(C5CCN(CCc6ccc([C@@H]7CCC(=O)NC7=O)cc6)CC5)nc4)cc23)ccc1[C@@H](C)NC(=O)c1noc(C(C)(C)C)n1]

Each enantiomer was separated from Example 117 by using preparative HPLC on a CHIRALPAK IA-3 with DCM:EtOH=40:60 as an eluent. The enantiomeric excesses were determined by using HPLC on a CHIRALPAK IA-3 with DCM:EtOH=40:60 as an eluent at a flow rate of 1.0 mL/min. The first enantiomer Example 152 was eluted at the retention time of 0.992 min, and the other enantiomer Example 153 was eluted at the retention time of 1.265 min. Example 152: ¹H NMR (400 MHz, CDCl₃) δ_{H} 12.00 (brs, 1H), 8.85 (s, 1H), 8.53-8.49 (m, 2H), 7.92-7.83 (m, 3H), 7.54 (d, J=8.0 Hz, 1H), 7.37 (d, J=8.0 Hz, 1H), 7.26-7.19 (m, 3H), 7.17-7.15 (m, 3H), 5.61-5.30 (m, 1H), 3.80-3.76 (m, 1H), 3.27-3.24 (m, 2H), 2.92 (brs, 3H), 2.77-2.63 (m, 4H), 2.55 (s, 3H), 2.29 (brs, 4H), 2.08-2.00 (m, 4H), 1.67 (d, J=6.8 Hz, 3H), 1.46 (s, 9H); [M+H]⁺=780.1. Example 153: ¹H NMR (400 MHz, CDCl₃) δ_{H} 11.60 (brs, 1H), 8.83 (s, 1H), 8.49 (s, 1H), 8.35 (s, 1H), 7.92-7.83 (m, 3H), 7.54 (d, J=8.0 Hz, 1H), 7.37 (d, J=8.0 Hz, 1H), 7.27-7.18 (m, 3H), 7.18-7.14 (m, 3H), 5.59-5.30 (m, 1H), 3.80-3.76 (m, 1H), 3.28-3.26 (m, 2H), 2.92 (brs, 3H), 2.77-2.66 (m, 4H), 2.55 (s, 3H), 2.29 (brs, 4H), 2.08-2.00 (m, 4H), 1.67 (d, J=6.8 Hz, 3H), 1.46 (s, 9H); [M+H]⁺=780.1.

### Example 154: 3-(tert-butyl)-N-((1R)-1-(4-(5-(4-(4-(2-(5-(2,6-dioxopiperidin-3-yl)-4-methylpyridin-2-yl)ethyl)piperazin-1-yl)phenyl)-1H-pyrazolo[3,4-b]pyridin-3-yl)-2-methylphenyl)ethyl)-1,2,4-oxadiazole-5-carboxamide

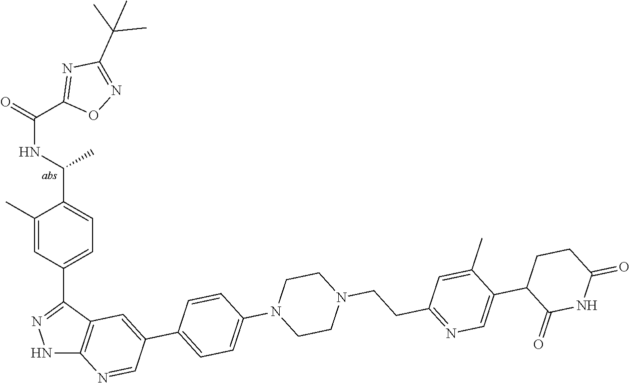 [SMILES: Cc1cc(CCN2CCN(c3ccc(-c4cnc5[nH]nc(-c6ccc([C@@H](C)NC(=O)c7nc(C(C)(C)C)no7)c(C)c6)c5c4)cc3)CC2)ncc1C1CCC(=O)NC1=O]

The titled compound was synthesized in the procedures similar to Example 1. ¹H NMR (400 MHz, DMSO) δ_{H} 13.76 (s, 1H), 10.89 (s, 1H), 9.94-9.87 (m, 1H), 8.80 (s, 1H), 8.56 (s, 1H), 8.26-8.14 (m, 2H), 7.94-7.78 (m, 2H), 7.73-7.53 (m, 3H), 7.16-6.99 (m, 3H), 5.39-5.28 (m, 1H), 4.05 (d, J=12.2 Hz, 1H), 3.20 (s, 4H), 2.95-2.51 (m, 12H), 2.42 (s, 3H), 2.28-2.13 (m, 1H), 2.04-1.89 (m, 1H), 1.56-1.47 (m, 3H), 1.40 (s, 9H); [M+H]⁺=795.6.

### Example 155: 5-(tert-butyl)-N-((1R)-1-(4-(5-(4-(4-(2-(5-(2,6-dioxopiperidin-3-yl)-6-methylpyridin-2-yl)ethyl)piperazin-1-yl)phenyl)-1H-pyrazolo[3,4-b]pyridin-3-yl)-2-methylphenyl)ethyl)-1,2,4-oxadiazole-3-carboxamide

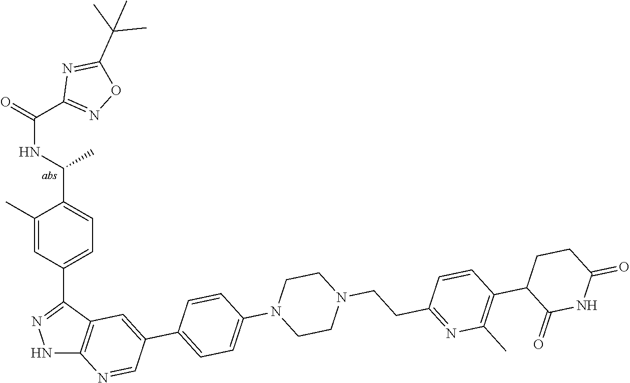 [SMILES: Cc1cc(-c2n[nH]c3ncc(-c4ccc(N5CCN(CCc6ccc(C7CCC(=O)NC7=O)c(C)n6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.76 (s, 1H), 10.87 (s, 1H), 9.52-9.43 (m, 1H), 8.80 (s, 1H), 8.56 (s, 1H), 7.94-7.78 (m, 2H), 7.73-7.53 (m, 3H), 7.43 (s, 1H), 7.16-6.99 (m, 3H), 5.39-5.28 (m, 1H), 4.05 (d, J=12.2 Hz, 1H), 3.20 (s, 4H), 2.95-2.51 (m, 14H), 2.42 (s, 3H), 2.28-2.13 (m, 1H), 2.04-1.89 (m, 1H), 1.56-1.47 (m, 3H), 1.34 (s, 9H), 1.27-1.16 (m, 1H); [M+H]⁺=795.6.

### Example 156: 3-(tert-butyl)-N-((1R)-1-(4-(5-(4-(4-(2-(5-(2,6-dioxopiperidin-3-yl)-6-methylpyridin-2-yl)ethyl)piperazin-1-yl)phenyl)-1H-pyrazolo[3,4-b]pyridin-3-yl)-2-methylphenyl)ethyl)-1,2,4-oxadiazole-5-carboxamide

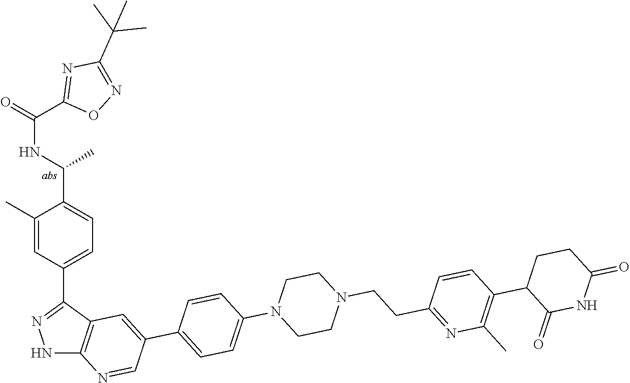 [SMILES: Cc1cc(-c2n[nH]c3ncc(-c4ccc(N5CCN(CCc6ccc(C7CCC(=O)NC7=O)c(C)n6)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.76 (s, 1H), 10.87 (s, 1H), 9.95-9.87 (m, 1H), 8.80 (s, 1H), 8.56 (s, 1H), 7.94-7.78 (m, 2H), 7.73-7.53 (m, 3H), 7.43 (s, 1H), 7.16-6.99 (m, 3H), 5.39-5.28 (m, 1H), 4.05 (d, J=12.2 Hz, 1H), 3.20 (s, 4H), 2.95-2.51 (m, 14H), 2.42 (s, 3H), 2.28-2.13 (m, 1H), 2.04-1.89 (m, 1H), 1.56-1.47 (m, 3H), 1.34 (s, 9H), 1.27-1.16 (m, 1H); [M+H]⁺=795.6.

### Example 157: (R)-5-(tert-butyl)-N-(1-(4-(6-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

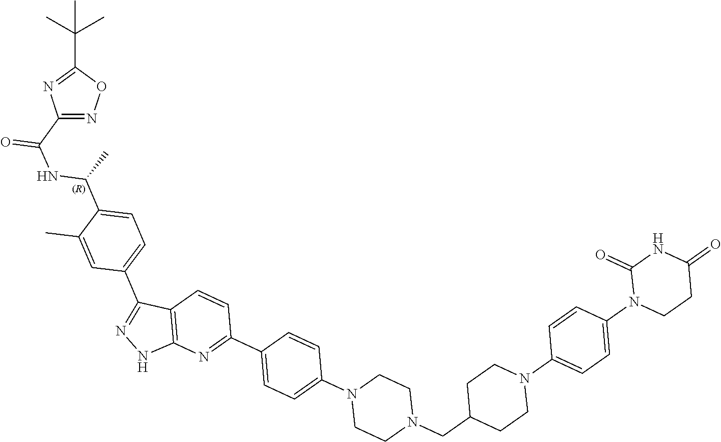

The titled compound was synthesized in the procedures similar to Example 1. ¹H NMR (400 MHz, DMSO) δ_{H} 13.65 (s, 1H), 10.27 (s, 1H), 9.50-9.48 (m, 1H), 8.56-8.54 (m, 1H), 8.07 (d, J=8.0 Hz, 2H), 7.81-7.68 (m, 3H), 7.63-7.60 (m, 1H), 7.13-7.09 (m, 4H), 6.95-6.93 (m, 2H), 5.38-5.34 (m, 1H), 3.71-3.68 (m, 4H), 3.35-3.33 (m, 2H), 3.34-3.33 (m, 3H), 3.32-3.30 (m, 2H), 2.70-2.65 (m, 3H), 2.65-2.53 (m, 6H), 2.50 (s, 3H), 2.25-2.23 (m, 1H), 1.83-1.80 (m, 1H), 1.80-1.78 (m, 1H), 1.52 (d, J=4.0 Hz, 3H), 1.42 (s, 9H); [M+H]⁺=850.8.

### Example 158: 5-(tert-butyl)-N-((1R)-1-(4-(5-(5-(5-((1-(4-(2,4-dioxotetrahydropyrimidin-1(2H)-yl)phenyl)piperidin-4-yl)methyl)hexahydropyrrolo[3,4-c]pyrrol-2(1H)-yl)pyridin-2-yl)-1H-pyrazolo[3,4-b]pyridin-3-yl)-2-methylphenyl)ethyl)-1,2,4-oxadiazole-3-carboxamide

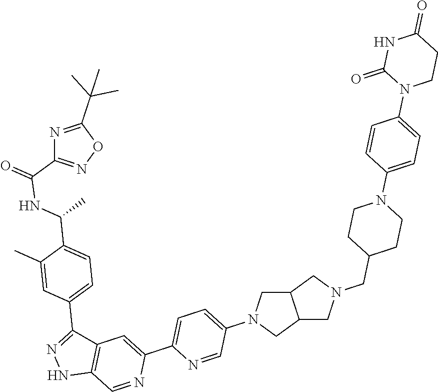 [SMILES: Cc1cc(-c2n[nH]c3cnc(-c4ccc(N5CC6CN(CC7CCN(c8ccc(N9CCC(=O)NC9=O)cc8)CC7)CC6C5)cn4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.80 (s, 1H), 10.26 (s, 1H), 9.52-9.50 (m, 1H), 9.22 (s, 1H), 8.99 (s, 1H), 8.17 (s, 1H), 8.02-7.99 (m, 2H), 7.83-7.82 (m, 1H), 7.82 (s, 1H), 7.64-7.62 (m, 1H), 7.11-7.09 (m, 3H), 6.91-6.89 (m, 2H), 5.38-5.34 (m, 1H), 3.71-3.68 (m, 4H), 3.67-3.65 (m, 2H), 3.15-3.13 (m, 2H), 2.95-2.93 (m, 2H), 2.66-2.58 (m, 4H), 2.55-2.51 (m, 4H), 2.52-2.48 (m, 3H), 2.30-2.25 (m, 2H), 1.91-1.88 (m, 2H), 1.85-1.80 (m, 2H), 1.52 (d, J=4.0 Hz, 3H), 1.42 (s, 9H); [M+H]⁺=877.7.

### Example 159: (R)-5-(tert-butyl)-N-(1-(4-(5-(4-(4-(2-(6-(2,4-dioxotetrahydropyrimidin-1(2H)-yl)pyridin-3-yl)ethyl)piperazin-1-yl)phenyl)-1H-pyrazolo[3,4-b]pyridin-3-yl)-2-methylphenyl)ethyl)-1,2,4-oxadiazole-3-carboxamide

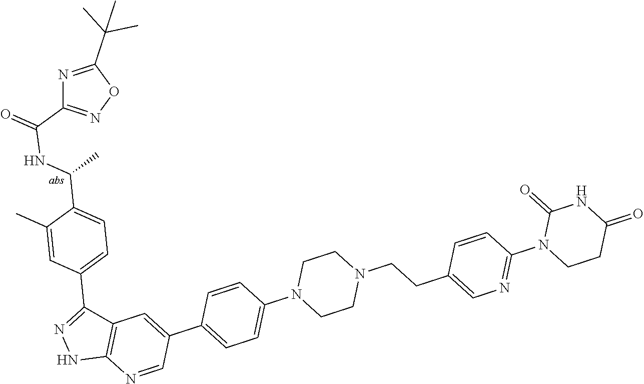 [SMILES: Cc1cc(-c2n[nH]c3ncc(-c4ccc(N5CCN(CCc6ccc(N7CCC(=O)NC7=O)nc6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.76 (s, 1H), 10.49 (s, 1H), 9.48 (s, 1H), 8.80 (s, 1H), 8.56 (s, 1H), 8.29 (s, 1H), 7.88 (s, 1H), 7.83 (s, 1H), 7.67 (brs, 4H), 7.59 (s, 1H), 7.06 (s, 2H), 5.34 (s, 1H), 4.00 (s, 2H), 3.19 (s, 4H), 2.78 (s, 2H), 2.63 (brs, 7H), 2.50 (brs, 4H), 1.49 (s, 3H), 1.40 (brs, 9H); [M+H]⁺=783.0.

### Example 160: 5-(tert-butyl)-N-((1R)-1-(4-(5-(4-(4-(2-(4-(2,4-dioxotetrahydropyrimidin-1(2H)-yl)phenyl)propyl)piperazin-1-yl)phenyl)-1H-pyrazolo[3,4-b]pyridin-3-yl)-2-methylphenyl)ethyl)-1,2,4-oxadiazole-3-carboxamide

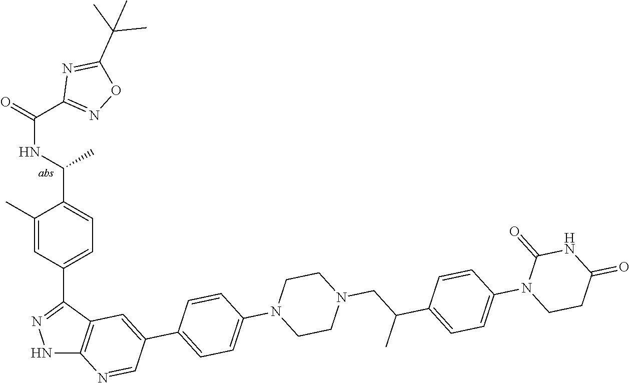 [SMILES: Cc1cc(-c2n[nH]c3ncc(-c4ccc(N5CCN(CC(C)c6ccc(N7CCC(=O)NC7=O)cc6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.76 (s, 1H), 10.34 (s, 1H), 9.47 (d, J=8.0 Hz, 1H), 8.81 (s, 1H), 8.57 (s, 1H), 7.91 (d, J=8.0 Hz, 1H), 7.84 (s, 1H), 7.68 (d, J=8.0 Hz, 2H), 7.60 (d, J=8.0 Hz, 1H), 7.27 (d, J=7.1 Hz, 4H), 7.05 (d, J=8.0 Hz, 2H), 5.36 (s, 1H), 3.77 (t, J=8.0 Hz, 2H), 3.19 (s, 4H), 3.06-2.98 (m, 1H), 2.70 (t, J=8.0 Hz, 2H), 2.64-2.52 (m, 7H), 2.48-2.44 (m, 2H), 1.51 (d, J=8.0 Hz, 3H), 1.42 (s, 9H), 1.26-1.20 (m, 3H); [M+H]⁺=796.0.

### Example 161 and 162: 5-(tert-butyl)-N—((R)-1-(4-(5-(5-(4-(4-((S)-2,6-dioxopiperidin-3-yl)phenethyl)piperazin-1-yl)pyridin-2-yl)-1H-pyrazolo[3,4-b]pyridin-3-yl)-2-methylphenyl)ethyl)-1,2,4-oxadiazole-3-carboxamide and 5-(tert-butyl)-N—((R)-1-(4-(5-(5-(4-(4-((R)-2,6-dioxopiperidin-3-yl)phenethyl)piperazin-1-yl)pyridin-2-yl)-1H-pyrazolo[3,4-b]pyridin-3-yl)-2-methylphenyl)ethyl)-1,2,4-oxadiazole-3-carboxamide

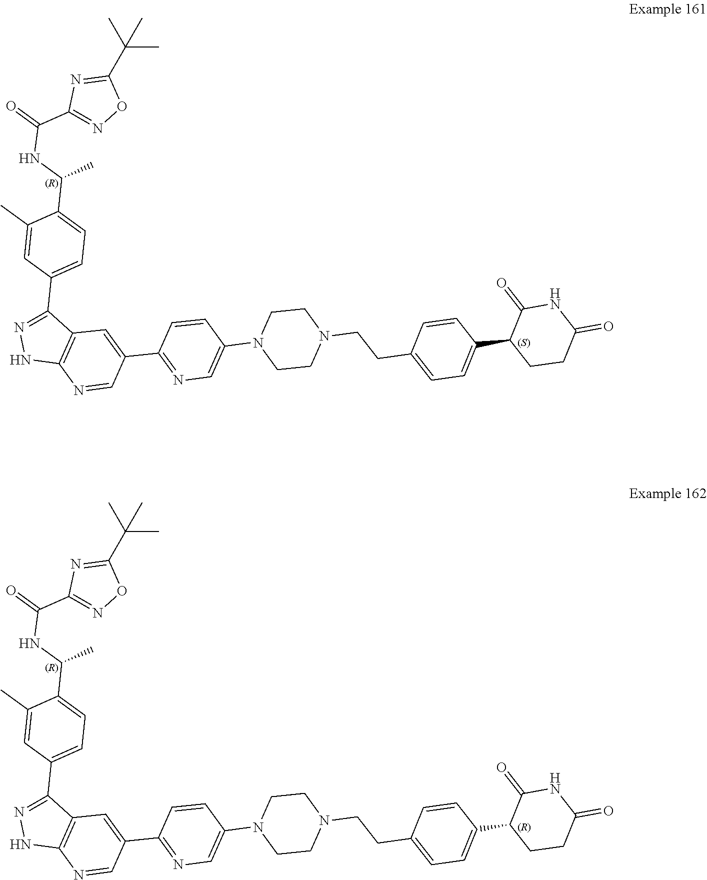

The Example 116 (30 mg) was separated by Prep-Chiral-HPLC with following conditions: Column: CHIRALPAK IA, 2*25 cm, 5 μm; Mobile Phase A: DCM, Mobile Phase B: EtOH; Flow rate: 15 mL/min; Gradient: 70% B to 70% B in 16 min; Detector: 220/254 nm; RT1: 7.185 min; RT2: 11.867 min; Sample Solvent: MeOH:DCM=1:1; Injection Volume: 2.5 mL; Number Of Runs: 1. This resulted in Example 161 (RT1: 1.644 min) (7.6 mg, 25.3%) and Example 162 (RT2: 1.161 min) (10.3 mg, 34.3%). Example 161: ¹H NMR (400 MHz, CDCl₃) δ_{H} 11.05 (s, 1H), 9.19 (s, 1H), 8.89 (s, 1H), 8.45 (s, 1H), 8.13 (s, 1H), 7.92 (s, 1H), 7.89-7.88 (m, 2H), 7.72 (d, J=8.0 Hz, 1H), 7.55 (d, J=8.0 Hz, 1H), 7.32 (d, J=8.0 Hz, 1H), 7.18-7.15 (m, 4H), 5.60 (s, 1H), 3.81-3.77 (m, 1H), 3.36 (s, 4H), 2.90-2.86 (m, 2H), 2.85-2.72 (m, 8H), 2.60 (s, 3H), 2.31-2.21 (m, 2H), 1.70-1.68 (m, 3H), 1.59 (s, 9H); [M+H]⁺=781.8. Example 162: ¹H NMR (400 MHz, CDCl₃) δ_{H} 11.08 (s, 1H), 9.19 (s, 1H), 8.89 (s, 1H), 8.45 (s, 1H), 8.13 (s, 1H), 7.92 (s, 1H), 7.89-7.88 (m, 2H), 7.72 (d, J=8.0 Hz, 1H), 7.55 (d, J=8.0 Hz, 1H), 7.32 (d, J=8.0 Hz, 1H), 7.18-7.15 (m, 4H), 5.63 (s, 1H), 3.81-3.77 (m, 1H), 3.36 (s, 4H), 2.90-2.86 (m, 2H), 2.85-2.72 (m, 8H), 2.60 (s, 3H), 2.31-2.21 (m, 2H), 1.70-1.68 (m, 3H), 1.59 (s, 9H); [M+H]⁺=781.8.

### Example 163: 5-(tert-butyl)-N-((1R)-1-(4-(5-(5-(4-(2-(5-(2,6-dioxopiperidin-3-yl)-6-methylpyridin-2-yl)ethyl)piperazin-1-yl)pyridin-2-yl)-1H-pyrazolo[3,4-b]pyridin-3-yl)-2-methylphenyl)ethyl)-1,2,4-oxadiazole-3-carboxamide

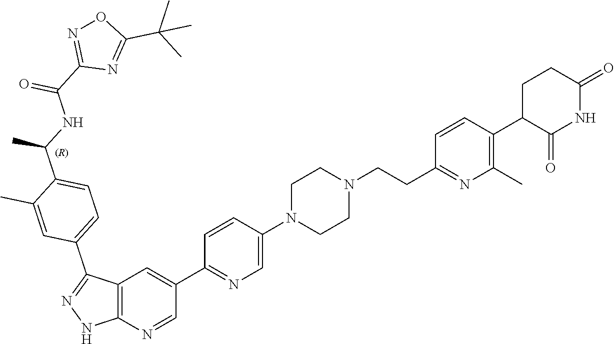

The titled compound was synthesized in the procedures similar to Example 1. ¹H NMR (400 MHz, DMSO) δ_{H} 13.79 (s, 1H), 10.88 (s, 1H), 9.49 (d, J=8.0 Hz, 1H), 9.20 (s, 1H), 8.94 (s, 1H), 8.44 (s, 1H), 8.02 (s, 1H), 7.88-7.86 (m, 2H), 7.63 (d, J=8.0 Hz, 1H), 7.42-7.40 (m, 2H), 7.09 (d, J=8.0 Hz, 1H), 5.38 (s, 1H), 4.10-4.08 (m, 1H), 3.26 (s, 4H), 2.89-2.84 (m, 2H), 2.79-2.54 (m, 11H), 2.42 (s, 3H), 2.25-2.16 (m, 1H), 1.98-1.93 (m, 1H), 1.50 (d, J=8.0 Hz, 3H), 1.40 (s, 9H); [M+H]⁺=796.7.

### Example 164: (R)-5-(tert-butyl)-N-(1-(4-(5-(4-(4-(2-(4-(2,4-dioxotetrahydropyrimidin-1(2H)-yl)-3,5-dimethyl-1H-pyrazol-1-yl)ethyl)piperazin-1-yl)phenyl)-1H-pyrazolo[3,4-b]pyridin-3-yl)-2-methylphenyl)ethyl)-1,2,4-oxadiazole-3-carboxamide

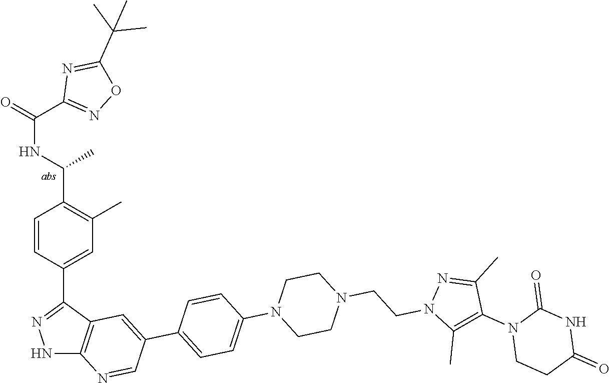 [SMILES: Cc1cc(-c2n[nH]c3ncc(-c4ccc(N5CCN(CCn6nc(C)c(N7CCC(=O)NC7=O)c6C)CC5)cc4)cc23)ccc1[C@@H](C)NC(=O)c1noc(C(C)(C)C)n1]

### Step 1: 1-(1-(2-hydroxyethyl)-3,5-dimethyl-1H-pyrazol-4-yl)-3-((2-(trimethylsilyl)ethoxy)methyl)dihydropyrimidine-2,4(1H,3H)-dione

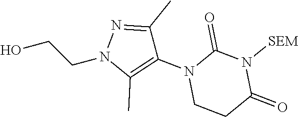 [SMILES: Cc1nn(CCO)c(C)c1N1CCC(=O)N(COCC[Si](C)(C)C)C1=O]

A mixture of 1-(3,5-dimethyl-1H-pyrazol-4-yl)-3-((2-(trimethylsilyl)ethoxy)methyl)-dihydropyrimidine-2,4(1H,3H)-dione (0.6 g, 1.77 mmol), 2-bromoethan-1-ol (4.4 g, 3.55 mmol) and DIPEA (0.46 g, 3.55 mmol) in NMP (20 mL) was stirred at 120° C. overnight. After the reaction was completed, the solvent was removed under reduced pressure and the crude product was purified with silica gel column chromatography (PE:EtOAc=10:1˜1:1 gradient elution) to give the product (0.7 g, 99%). [M+H]⁺=383.5.

### Step 2: 2-(4-(2,4-dioxo-3-((2-(trimethylsilyl)ethoxy)methyl)tetrahydropyrimidin-1(2H)-yl)-3,5-dimethyl-1H-pyrazol-1-yl)ethyl methanesulfonate

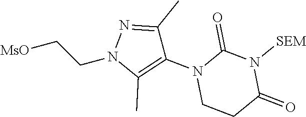 [SMILES: Cc1nn(CCOS(C)(=O)=O)c(C)c1N1CCC(=O)N(COCC[Si](C)(C)C)C1=O]

To a solution of 1-(1-(2-hydroxyethyl)-3,5-dimethyl-1H-pyrazol-4-yl)-3-((2-(trimethylsilyl)ethoxy)methyl)dihydropyrimidine-2,4(1H,3H)-dione (0.7 g, 1.83 mmol) and Et₃N (0.37 g, 3.66 mmol) in DCM (10 mL) was added MsCl (0.42 g, 3.66 mmol). The resulting mixture was stirred at room temperature overnight. The reaction was quenched with water and extracted with EtOAc. The combined organic layers were washed with brine, dried over anhydrous Na₂SO₄, and evaporated in vacuum to afford the crude product, which was further purified with silica gel column chromatography (PE:EtOAc=10:1˜3:1 gradient elution) to give the product (0.4 g, 47.6%). [M+H]⁺=461.6.

### Step 3: (R)-5-(tert-butyl)-N-(1-(4-(5-(4-(4-(2-(4-(2,4-dioxo-3-((2-(trimethylsilyl)ethoxy)methyl)tetrahydropyrimidin-1(2H)-yl)-3,5-dimethyl-1H-pyrazol-1-yl)ethyl)piperazin-1-yl)phenyl)-1H-pyrazolo[3,4-b]pyridin-3-yl)-2-methylphenyl)ethyl)-1,2,4-oxadiazole-3-carboxamide

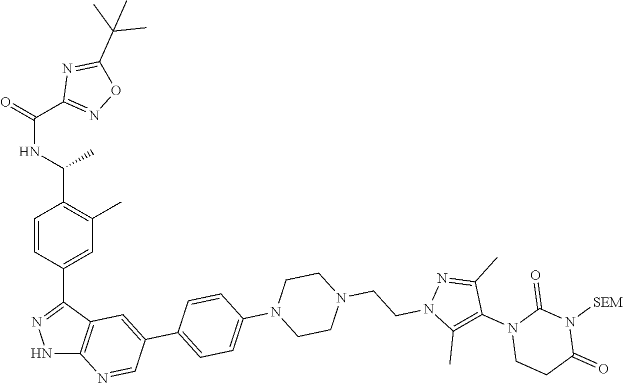 [SMILES: Cc1cc(-c2n[nH]c3ncc(-c4ccc(N5CCN(CCn6nc(C)c(N7CCC(=O)N(COCC[Si](C)(C)C)C7=O)c6C)CC5)cc4)cc23)ccc1[C@@H](C)NC(=O)c1noc(C(C)(C)C)n1]

A mixture of (R)-5-(tert-butyl)-N-(1-(2-methyl-4-(5-(4-(piperazin-1-yl)phenyl)-1H-pyrazolo[3,4-b]pyridin-3-yl)phenyl)ethyl)-1,2,4-oxadiazole-3-carboxamide (60 mg, 0.106 mmol), 2-(4-(2,4-dioxo-3-((2-(trimethylsilyl)ethoxy)methyl)tetrahydropyrimidin-1(2H)-yl)-3,5-dimethyl-1H-pyrazol-1-yl)ethyl methanesulfonate (58.7 mg, 0.13 mmol), DIPEA (27 mg, 0.21 mmol) and KI (34.8 mg, 0.21 mmol) in MeCN (10 mL) was stirred in a round bottom flask at 80° C. for 8 h under N₂. The solvent was removed under reduced pressure and the crude product was purified with silica gel column chromatography (DCM:MeOH=100:1˜10:1 gradient elution) to give the product (390 mg, 53%). [M+H]⁺=929.5.

### Step 4: (R)-5-(tert-butyl)-N-(1-(4-(5-(4-(4-(2-(4-(3-(hydroxymethyl)-2,4-dioxotetrahydropyrimidin-1(2H)-yl)-3,5-dimethyl-1H-pyrazol-1-yl)ethyl)piperazin-1-yl)phenyl)-1H-pyrazolo[3,4-b]pyridin-3-yl)-2-methylphenyl)ethyl)-1,2,4-oxadiazole-3-carboxamide

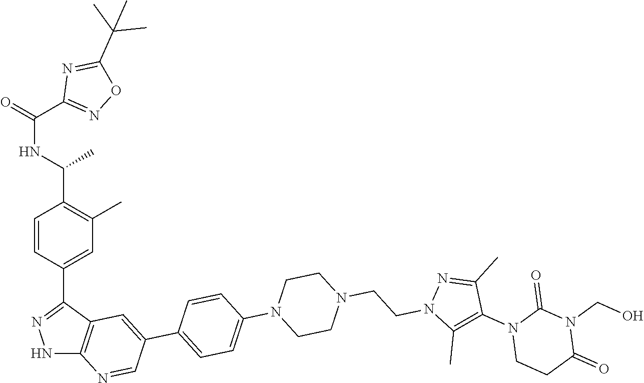 [SMILES: Cc1cc(-c2n[nH]c3ncc(-c4ccc(N5CCN(CCn6nc(C)c(N7CCC(=O)N(CO)C7=O)c6C)CC5)cc4)cc23)ccc1[C@@H](C)NC(=O)c1noc(C(C)(C)C)n1]

A mixture of (R)-5-(tert-butyl)-N-(1-(4-(5-(4-(4-(2-(4-(2,4-dioxo-3-((2-(trimethylsilyl)ethoxy)methyl)tetrahydropyrimidin-1(2H)-yl)-3,5-dimethyl-1H-pyrazol-1-yl)ethyl)piperazin-1-yl)phenyl)-1H-pyrazolo[3,4-b]pyridin-3-yl)-2-methylphenyl)ethyl)-1,2,4-oxadiazole-3-carboxamide (0.39 g, 0.42 mmol) and trifluoroacetic acid (20 mL) in dichloromethane (20 mL) was stirred in a round bottom flask at room temperature overnight. The mixture was evaporated in vacuum to afford the crude product (0.3 g, 85%), which was used for next step without further purification. [M+H]⁺=829.4.

### Step 5: (R)-5-(tert-butyl)-N-(1-(4-(5-(4-(4-(2-(4-(2,4-dioxotetrahydropyrimidin-1(2H)-yl)-3,5-dimethyl-1H-pyrazol-1-yl)ethyl)piperazin-1-yl)phenyl)-1H-pyrazolo[3,4-b]pyridin-3-yl)-2-methylphenyl)ethyl)-1,2,4-oxadiazole-3-carboxamide

To a stirred solution of (R)-5-(tert-butyl)-N-(1-(4-(5-(4-(4-(2-(4-(3-(hydroxymethyl)-2,4-dioxotetrahydropyrimidin-1(2H)-yl)-3,5-dimethyl-1H-pyrazol-1-yl)ethyl)piperazin-1-yl)phenyl)-1H-pyrazolo[3,4-b]pyridin-3-yl)-2-methylphenyl)ethyl)-1,2,4-oxadiazole-3-carboxamide (0.3 g, 0.36 mmol) in MeOH (10 mL) was added NH₃/H₂O (2 mL). The mixture was allowed to stir at 0° C. for 30 min. LCMS showed the reaction was completed. The mixture was evaporated in vacuum to afford the crude product, which was further purified with silica gel column chromatography (DCM:MeOH=10:1˜2:1 gradient elution) to give the product (60 mg, 20%). ¹H NMR (400 MHz, DMSO) δ_{H} 13.76 (s, 1H), 10.28 (s, 1H), 9.50-9.44 (m, 1H), 8.81-8.76 (m, 1H), 8.58-8.54 (m, 1H), 7.92-7.86 (m, 1H), 7.84-7.80 (m, 1H), 7.70-7.64 (m, 2H), 7.60-7.55 (m, 1H), 7.08-7.02 (m, 2H), 5.40-5.28 (m, 1H), 4.10-4.00 (m, 2H), 3.60-3.45 (m, 2H), 3.22-3.14 (m, 4H), 2.75-2.63 (m, 4H), 2.62-2.52 (m, 4H), 2.16-2.10 (m, 4H), 2.02-1.96 (m, 4H), 1.49 (s, 3H), 1.40 (s, 9H); [M+H]⁺=799.9.

### Example 165: (R)-5-(tert-butyl)-N-(1-(4-(5-(4-(4-((4-(5-(2,4-dioxotetrahydropyrimidin-1(2H)-yl)pyridin-2-yl)piperazin-1-yl)methyl)piperidin-1-yl)phenyl)-1H-pyrazolo[3,4-b]pyridin-3-yl)-2-methylphenyl)ethyl)-1,2,4-oxadiazole-3-carboxamide

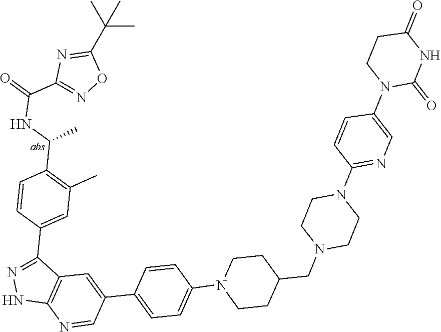

The titled compound was synthesized in the procedures similar to Example 1. ¹H NMR (400 MHz, DMSO) δ_{H} 13.76 (s, 1H), 10.35 (s, 1H), 9.48 (d, J=8.0 Hz, 1H), 8.79 (d, J=2.4 Hz, 1H), 8.55 (d, J=1.6 Hz, 1H), 8.05 (d, J=2.4 Hz, 1H), 7.89 (d, J=8.4 Hz, 1H), 7.83 (s, 1H), 7.66 (s, 1H), 7.64 (s, 1H), 7.58 (d, J=8.4 Hz, 1H), 7.49 (dd, J=8.8, 2.4 Hz, 1H), 7.04 (d, J=8.8 Hz, 2H), 6.83 (d, J=8.8 Hz, 1H), 5.37-5.27 (m, 1H), 3.77 (d, J=12.0 Hz, 2H), 3.68 (t, J=6.8 Hz, 2H), 3.48 (brs, 4H), 2.78-2.62 (m, 4H), 2.50 (s, 3H), 2.44 (s, 4H), 2.20 (d, J=6.8 Hz, 2H), 1.82 (d, J=12.0 Hz, 2H), 1.73 (brs, 1H), 1.49 (d, J=7.2 Hz, 3H), 1.40 (s, 9H), 1.24-1.21 (m, 2H); [M+H]⁺=851.7.

### Example 166: 5-(tert-butyl)-N-((1R)-1-(4-(5-(4-(4-(2-(5-(2,6-dioxopiperidin-3-yl)-6-methylpyridin-2-yl)ethyl)piperazin-1-yl)phenyl)-1H-pyrazolo[3,4-b]pyridin-3-yl)-3-fluoro-2-methylphenyl)ethyl)-1,2,4-oxadiazole-3-carboxamide

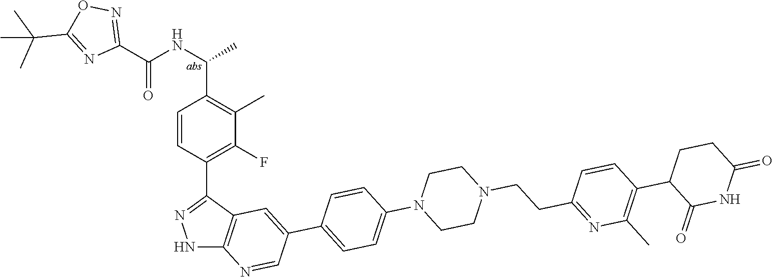 [SMILES: Cc1nc(CCN2CCN(c3ccc(-c4cnc5[nH]nc(-c6ccc([C@@H](C)NC(=O)c7noc(C(C)(C)C)n7)c(C)c6F)c5c4)cc3)CC2)ccc1C1CCC(=O)NC1=O]

The titled compound was synthesized in the procedures similar to Example 1. ¹H NMR (400 MHz, DMSO) δ_{H} 13.96 (s, 1H), 10.89 (s, 2H), 9.57 (d, J=8.0 Hz, 1H), 8.83 (s, 1H), 8.31 (s, 1H), 7.79-7.60 (m, 3H), 7.57-7.47 (m, 1H), 7.42 (d, J=8.0 Hz, 1H), 7.14 (s, 5H), 5.41-5.28 (m, 1H), 4.06 (s, 1H), 3.48 (s, 5H), 3.25-3.09 (m, 4H), 2.81-2.69 (m, 2H), 2.57-2.52 (m, 1H), 2.51-2.49 (m, 1H), 2.44 (s, 3H), 2.38 (d, J=2.0 Hz, 3H), 2.23-2.16 (m, 2H), 1.99-1.92 (m, 1H), 1.50 (d, J=8.0 Hz, 3H), 1.41 (s, 9H); [M+H]⁺=813.6.

### Example 167: 5-(tert-butyl)-N-((1R)-1-(4-(5-(4-(4-(2-(5-(2,6-dioxopiperidin-3-yl)-6-methylpyridin-2-yl)ethyl)piperazin-1-yl)phenyl)-1H-pyrazolo[3,4-b]pyridin-3-yl)-2-(hydroxymethyl)phenyl)ethyl)-1,2,4-oxadiazole-3-carboxamide

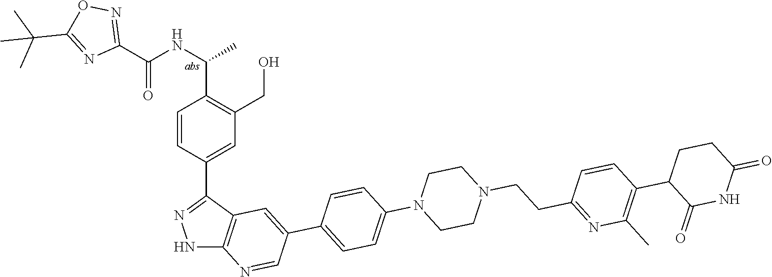 [SMILES: Cc1nc(CCN2CCN(c3ccc(-c4cnc5[nH]nc(-c6ccc([C@@H](C)NC(=O)c7noc(C(C)(C)C)n7)c(CO)c6)c5c4)cc3)CC2)ccc1C1CCC(=O)NC1=O]

The titled compound was synthesized in the procedures similar to Example 1. ¹H NMR (400 MHz, DMSO) δ_{H} 13.80 (s, 1H), 10.89 (s, 1H), 9.53 (d, J=8.0 Hz, 1H), 8.82 (d, J=4.0 Hz, 1H), 8.58 (s, 1H), 8.04 (s, 1H), 7.96 (d, J=8.0 Hz, 1H), 7.70 (s, 2H), 7.64 (d, J=8.0 Hz, 1H), 7.53 (s, 1H), 7.15 (s, 3H), 5.43-5.28 (m, 2H), 4.86-4.68 (m, 2H), 4.07 (d, J=8.0 Hz, 1H), 3.50 (s, 5H), 3.23-3.14 (m, 3H), 3.11-3.03 (m, 2H), 2.80-2.70 (m, 2H), 2.56-2.51 (m, 1H), 2.44-2.40 (m, 3H), 2.29-2.16 (m, 2H), 2.00-1.90 (m, 1H), 1.51 (d, J=8.0 Hz, 3H), 1.40 (s, 9H); [M+H]⁺=811.6.

### Example 168: 5-(tert-butyl)-N-((1R)-1-(4-(5-(4-(4-(2-(1-(4-(2,6-dioxopiperidin-3-yl)-3-methylphenyl)piperidin-4-yl)ethyl)piperazin-1-yl)phenyl)-1H-pyrazolo[3,4-b]pyridin-3-yl)-2-methylphenyl)ethyl)-1,2,4-oxadiazole-3-carboxamide

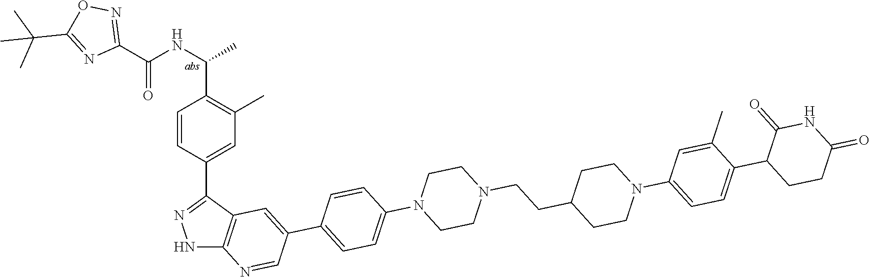 [SMILES: Cc1cc(N2CCC(CCN3CCN(c4ccc(-c5cnc6[nH]nc(-c7ccc([C@@H](C)NC(=O)c8noc(C(C)(C)C)n8)c(C)c7)c6c5)cc4)CC3)CC2)ccc1C1CCC(=O)NC1=O]

The titled compound was synthesized in the procedures similar to Example 1. ¹H NMR (400 MHz, DMSO) δ_{H} 13.77 (s, 1H), 10.76 (s, 1H), 9.48 (d, J=8.0 Hz, 1H), 8.80 (s, 1H), 8.57 (s, 1H), 7.89 (d, J=8.0 Hz, 1H), 7.83 (s, 1H), 7.69 (s, 2H), 7.58 (d, J=8.0 Hz, 1H), 7.07 (s, 2H), 6.88 (d, J=8.0 Hz, 1H), 6.76-6.63 (m, 2H), 5.39-5.28 (m, 1H), 3.87 (dd, J=12.0, 4.0 Hz, 1H), 3.63 (d, J=12.0 Hz, 2H), 3.35 (s, 3H), 3.18 (s, 2H), 2.75-2.60 (m, 3H), 2.60-2.54 (m, 3H), 2.50 (s, 3H), 2.46-2.41 (m, 1H), 2.40-2.31 (m, 1H), 2.18 (s, 5H), 2.13-2.04 (m, 1H), 1.97-1.89 (m, 1H), 1.75 (d, J=12.0 Hz, 2H), 1.49 (d, J=8.0 Hz, 4H), 1.46-1.36 (m, 11H), 1.30-1.22 (m, 3H); [M+H]⁺=877.9.

### Example 169: 5-(tert-butyl)-N-((1R)-1-(4-(5-(4-(4-(2-(1-(4-(2,6-dioxopiperidin-3-yl)-3-methoxyphenyl)piperidin-4-yl)ethyl)piperazin-1-yl)phenyl)-1H-pyrazolo[3,4-b]pyridin-3-yl)-2-methylphenyl)ethyl)-1,2,4-oxadiazole-3-carboxamide

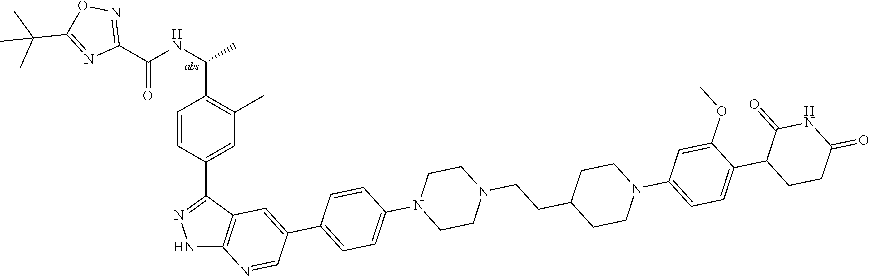 [SMILES: COc1cc(N2CCC(CCN3CCN(c4ccc(-c5cnc6[nH]nc(-c7ccc([C@@H](C)NC(=O)c8noc(C(C)(C)C)n8)c(C)c7)c6c5)cc4)CC3)CC2)ccc1C1CCC(=O)NC1=O]

The titled compound was synthesized in the procedures similar to Example 1. ¹H NMR (400 MHz, DMSO) δ_{H} 13.76 (s, 1H), 10.67 (s, 1H), 9.49 (s, 1H), 8.79 (s, 1H), 8.56 (s, 1H), 7.88 (s, 1H), 7.82 (s, 1H), 7.68 (s, 2H), 7.59 (s, 1H), 7.05 (s, 2H), 6.87 (s, 1H), 6.51 (s, 1H), 6.43 (s, 1H), 5.34 (s, 1H), 3.74 (s, 1H), 3.69-3.59 (m, 5H), 3.19 (s, 4H), 2.64 (t, J=12.0 Hz, 4H), 2.51 (s, 5H), 2.42-2.32 (m, 3H), 2.17-2.06 (m, 1H), 1.90-1.80 (m, 1H), 1.75 (d, J=8.0 Hz, 2H), 1.52-1.42 (m, 6H), 1.41-1.36 (m, 9H), 1.32-1.20 (m, 3H); [M+H]⁺=894.0.

### Example 170: 5-(tert-butyl)-N-((1R)-1-(4-(5-(5-(5-(2-(5-(2,6-dioxopiperidin-3-yl)-6-methylpyridin-2-yl)ethyl)-2,5-diazabicyclo[2.2.1]heptan-2-yl)pyridin-2-yl)-1H-pyrazolo[3,4-b]pyridin-3-yl)-2-methylphenyl)ethyl)-1,2,4-oxadiazole-3-carboxamide

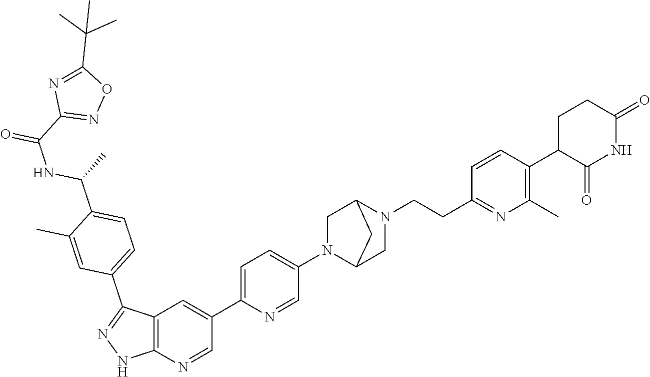 [SMILES: Cc1cc(-c2n[nH]c3ncc(-c4ccc(N5CC6CC5CN6CCc5ccc(C6CCC(=O)NC6=O)c(C)n5)cn4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.80 (s, 1H), 10.84 (s, 1H), 9.50-9.48 (m, 1H), 9.18 (s, 1H), 8.86 (s, 1H), 8.23-8.19 (m, 1H), 8.11 (s, 1H), 7.92-7.85 (m, 2H), 7.81 (s, 1H), 7.74-7.70 (m, 1H), 7.39-7.36 (m, 1H), 7.06-7.02 (m, 1H), 5.36-5.32 (m, 1H), 4.02-3.98 (m, 1H), 3.34-3.33 (m, 4H), 3.30-3.28 (m, 4H), 3.28-3.10 (m, 4H), 3.10-3.08 (m, 1H), 2.75-2.70 (m, 4H), 2.50 (s, 3H), 2.48-2.44 (m, 2H), 2.44-2.42 (m, 2H), 2.35-2.22 (m, 4H), 2.21-1.98 (m, 4H), 1.52 (d, J=4.0 Hz, 3H), 1.42 (s, 9H); [M+H]⁺=808.7.

### Example 171: (R)-5-(tert-butyl)-N-(1-(4-(5-(4-(4-((1-(4-(2,4-dioxotetrahydropyrimidin-1(2H)-yl)-2-methoxyphenyl)piperidin-4-yl)methyl)piperazin-1-yl)phenyl)-1H-pyrazolo[3,4-b]pyridin-3-yl)-2-methylphenyl)ethyl)-1,2,4-oxadiazole-3-carboxamide

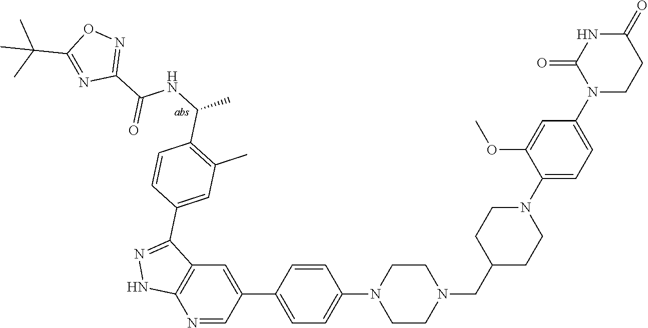 [SMILES: COc1cc(N2CCC(=O)NC2=O)ccc1N1CCC(CN2CCN(c3ccc(-c4cnc5[nH]nc(-c6ccc([C@@H](C)NC(=O)c7noc(C(C)(C)C)n7)c(C)c6)c5c4)cc3)CC2)CC1]

The titled compound was synthesized in the procedures similar to Example 1. ¹H NMR (400 MHz, DMSO) δ_{H} 13.76 (s, 1H), 10.29 (s, 1H), 9.47 (d, J=8.0 Hz, 1H), 8.82 (s, 1H), 8.58 (s, 1H), 7.91 (d, J=8.0 Hz, 1H), 7.85 (s, 1H), 7.69 (d, J=8.0 Hz, 2H), 7.60 (d, J=8.0 Hz, 1H), 7.07 (d, J=8.0 Hz, 2H), 6.93-6.85 (m, 2H), 6.81 (d, J=8.0 Hz, 1H), 5.36 (s, 1H), 3.80-3.69 (m, 5H), 3.37 (s, 4H), 3.22 (s, 4H), 2.69 (s, 2H), 2.54 (s, 7H), 2.26 (s, 2H), 1.81 (d, J=12.0 Hz, 2H), 1.68 (s, 1H), 1.51 (d, J=4.0 Hz, 3H), 1.43 (s, 9H), 1.36-1.21 (m, 2H); [M+H]⁺=880.7.

### Example 172: (R)-5-(tert-butyl)-N-(1-(4-(5-(4-(4-((1-(4-(2,4-dioxotetrahydropyrimidin-1(2H)-yl)-3-methylphenyl)piperidin-4-yl)methyl)piperazin-1-yl)phenyl)-1H-pyrazolo[3,4-b]pyridin-3-yl)-2-methylphenyl)ethyl)isoxazole-3-carboxamide

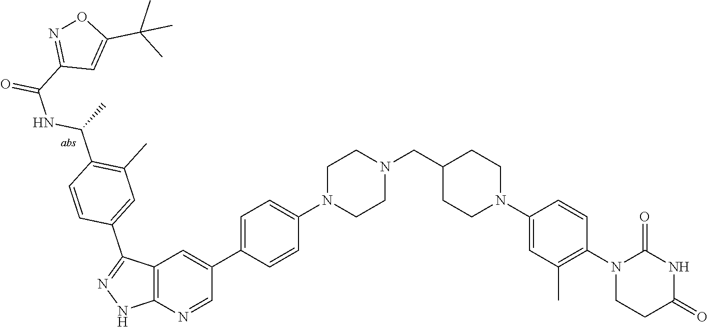 [SMILES: Cc1cc(-c2n[nH]c3ncc(-c4ccc(N5CCN(CC6CCN(c7ccc(N8CCC(=O)NC8=O)c(C)c7)CC6)CC5)cc4)cc23)ccc1[C@@H](C)NC(=O)c1cc(C(C)(C)C)on1]

The titled compound was synthesized in the procedures similar to Example 1. ¹H NMR (400 MHz, DMSO) δ_{H} 13.77 (s, 1H), 10.25 (s, 1H), 9.25 (d, J=7.5 Hz, 1H), 8.82 (s, 1H), 8.58 (s, 1H), 7.90 (d, J=8.8 Hz, 1H), 7.83 (s, 1H), 7.69 (d, J=7.6 Hz, 2H), 7.59 (d, J=8.0 Hz, 1H), 7.13-7.00 (m, 3H), 6.86-6.72 (m, 2H), 6.56 (s, 1H), 5.34 (s, 1H), 3.75-3.63 (m, 3H), 3.50-3.43 (m, 1H), 3.26-3.16 (m, 4H), 2.82-2.60 (m, 5H), 2.58-2.52 (m, 5H), 2.29-2.20 (m, 2H), 2.12 (s, 3H), 1.86-1.77 (m, 2H), 1.77-1.67 (m, 1H), 1.48 (d, J=6.4 Hz, 3H), 1.32 (s, 9H), 1.24-1.20 (m, 3H); [M+H]⁺=864.0.

### Example 173: 5-(tert-butyl)-N-((1R)-1-(4-(5-(5-(5-(2-(5-(2,6-dioxopiperidin-3-yl)-4-methylpyridin-2-yl)ethyl)-2,5-diazabicyclo[2.2.1]heptan-2-yl)pyridin-2-yl)-1H-pyrazolo[3,4-b]pyridin-3-yl)-2-methylphenyl)ethyl)-1,2,4-oxadiazole-3-carboxamide

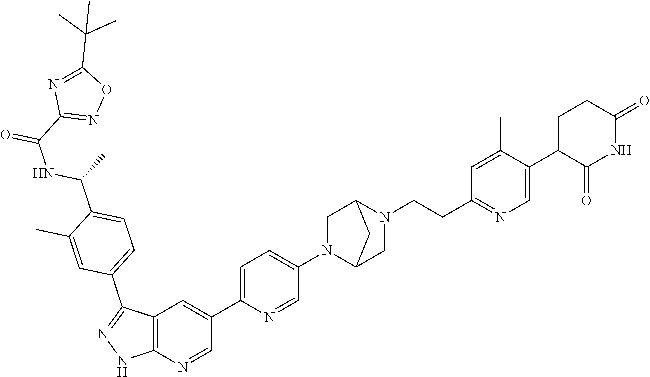 [SMILES: Cc1cc(CCN2CC3CC2CN3c2ccc(-c3cnc4[nH]nc(-c5ccc([C@@H](C)NC(=O)c6noc(C(C)(C)C)n6)c(C)c5)c4c3)nc2)ncc1C1CCC(=O)NC1=O]

The titled compound was synthesized in the procedures similar to Example 1. ¹H NMR (400 MHz, DMSO) δ_{H} 13.80 (s, 1H), 9.49 (s, 1H), 9.00 (s, 1H), 8.88 (s, 1H), 8.25-8.20 (m, 4H), 7.90-7.85 (m, 2H), 7.65-7.60 (m, 2H), 7.24-7.22 (m, 1H), 6.68-6.65 (m, 1H), 5.32-5.30 (m, 1H), 3.65-3.60 (m, 4H), 3.60-3.58 (m, 1H), 3.32-3.30 (m, 3H), 3.30-3.25 (m, 5H), 2.60 (s, 1H), 2.50 (s, 3H), 2.25-2.21 (m, 1H), 2.03-1.96 (m, 3H), 1.86-1.82 (m, 1H), 1.52 (d, J=4.0 Hz, 3H), 1.42 (s, 9H); [M+H]⁺=807.9.

### Example 174: 5-(tert-butyl)-N-((1R)-1-(4-(5-(5-(5-(4-(2,6-dioxopiperidin-3-yl)phenethyl)hexahydropyrrolo[3,4-c]pyrrol-2(1H)-yl)pyridin-2-yl)-1H-pyrazolo[3,4-b]pyridin-3-yl)-2-methylphenyl)ethyl)-1,2,4-oxadiazole-3-carboxamide

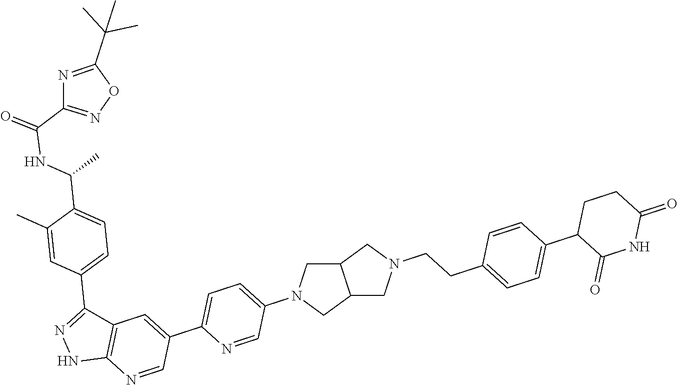 [SMILES: Cc1cc(-c2n[nH]c3ncc(-c4ccc(N5CC6CN(CCc7ccc(C8CCC(=O)NC8=O)cc7)CC6C5)cn4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.79 (s, 1H), 10.79 (s, 1H), 9.47 (d, J=8.0 Hz, 1H), 9.22 (s, 1H), 8.90 (s, 1H), 8.20 (s, 1H), 8.04 (d, J=8.0 Hz, 1H), 7.90 (d, J=8.0 Hz, 1H), 7.83 (s, 1H), 7.62 (d, J=8.0 Hz, 1H), 7.18-7.08 (m, 5H), 5.39-5.35 (m, 1H), 3.75-3.70 (m, 2H), 3.40-3.35 (m, 3H), 3.30-3.25 (m, 3H), 2.90-2.86 (m, 3H), 2.84-2.82 (m, 4H), 2.76-2.70 (m, 4H), 2.50 (s, 3H), 2.20-2.16 (m, 2H), 2.02-1.96 (m, 2H), 1.52 (d, J=4.0 Hz, 3H), 1.42 (s, 9H); [M+H]⁺=807.8.

### Example 175: 5-(tert-butyl)-N-((1R)-1-(4-(5-(4-(4-(2-(4-(2,6-dioxopiperidin-3-yl)phenyl)-2,2-difluoroethyl)piperazin-1-yl)phenyl)-1H-pyrazolo[3,4-b]pyridin-3-yl)-2-methylphenyl)ethyl)-1,2,4-oxadiazole-3-carboxamide

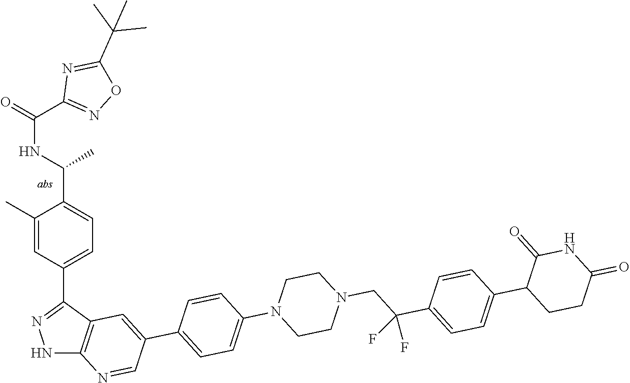 [SMILES: Cc1cc(-c2n[nH]c3ncc(-c4ccc(N5CCN(CC(F)(F)c6ccc(C7CCC(=O)NC7=O)cc6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.78 (s, 1H), 10.90 (s, 1H), 9.92 (d, J=6.3 Hz, 1H), 9.47 (d, J=8.0 Hz, 1H), 8.81 (s, 1H), 8.58 (s, 1H), 7.96-7.83 (m, 2H), 7.72-7.52 (m, 5H), 7.36 (d, J=7.0 Hz, 2H), 7.04 (d, J=8.1 Hz, 2H), 5.40-5.31 (m, 1H), 4.01-3.91 (m, 1H), 3.24-3.08 (m, 7H), 2.78-2.53 (m, 7H), 2.31-2.18 (m, 1H), 2.11-2.01 (m, 1H), 1.53 (d, J=5.9 Hz, 3H), 1.42 (s, 9H); [M+H]⁺=816.9.

### Example 176: 5-(tert-butyl)-N-((1R)-1-(4-(5-(4-(4-(3-(4-(2,6-dioxopiperidin-3-yl)phenyl)cyclobutyl)piperazin-1-yl)phenyl)-1H-pyrazolo[3,4-b]pyridin-3-yl)-2-methylphenyl)ethyl)-1,2,4-oxadiazole-3-carboxamide formate

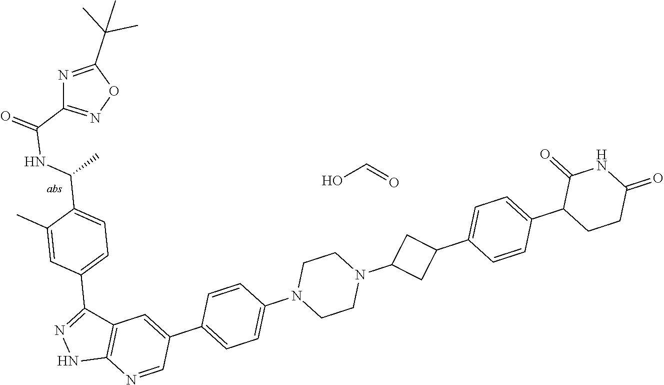 [SMILES: Cc1cc(-c2n[nH]c3ncc(-c4ccc(N5CCN(C6CC(c7ccc(C8CCC(=O)NC8=O)cc7)C6)CC5)cc4)cc23)ccc1[C@@H](C)NC(=O)c1noc(C(C)(C)C)n1 ; O=CO]

The titled compound was synthesized in the procedures similar to Example 1. ¹H NMR (400 MHz, DMSO) δ_{H} 13.78 (s, 1H), 10.83 (s, 1H), 9.49 (d, J=8.0 Hz, 1H), 8.82 (s, 1H), 8.58 (s, 1H), 7.91 (d, J=7.2 Hz, 1H), 7.85 (s, 1H), 7.69 (d, J=8.4 Hz, 2H), 7.60 (d, J=8.0 Hz, 1H), 7.21 (d, J=7.6 Hz, 2H), 7.15 (d, J=7.6 Hz, 2H), 7.07 (d, J=8.4 Hz, 2H), 5.38-5.35 (m, 1H), 3.85-3.79 (m, 1H), 3.22-3.15 (m, 6H), 2.81-2.61 (m, 2H), 2.50 (brs, 8H), 2.19-2.16 (m, 1H), 2.03 (brs, 1H), 1.90-1.88 (m, 2H), 1.51 (d, J=6.4 Hz, 3H), 1.42 (s, 9H); [M+H]⁺=806.9.

### Example 177: 3-(tert-butyl)-N-((1R)-1-(4-(5-(4-(4-(2-(5-(2,6-dioxopiperidin-3-yl)-6-methylpyridin-2-yl)ethyl)piperazin-1-yl)phenyl)-1H-pyrazolo[3,4-b]pyridin-3-yl)-2-(hydroxymethyl)phenyl)ethyl)-1,2,4-oxadiazole-5-carboxamide

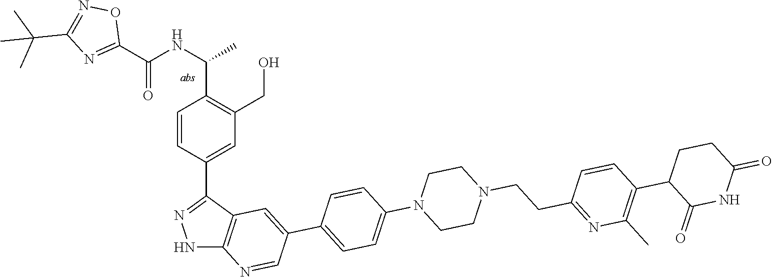 [SMILES: Cc1nc(CCN2CCN(c3ccc(-c4cnc5[nH]nc(-c6ccc([C@@H](C)NC(=O)c7nc(C(C)(C)C)no7)c(CO)c6)c5c4)cc3)CC2)ccc1C1CCC(=O)NC1=O]

The titled compound was synthesized in the procedures similar to Example 1. ¹H NMR (400 MHz, DMSO) δ_{H} 13.81 (s, 1H), 10.90 (s, 1H), 9.95 (s, 1H), 8.84 (s, 1H), 8.60 (s, 1H), 8.03 (d, J=28.0 Hz, 2H), 7.69 (s, 3H), 7.58-7.38 (m, 1H), 7.16 (s, 3H), 5.35 (s, 2H), 4.80 (d, J=32.0 Hz, 2H), 4.09 (s, 1H), 3.51 (s, 5H), 3.24-3.11 (m, 3H), 2.96-2.58 (m, 5H), 2.41-2.34 (m, 2H), 1.97 (s, 1H), 1.55 (s, 3H), 1.36 (s, 9H), 1.23 (s, 2H); [M+H]⁺=811.6.

### Example 178: (R)-5-(tert-butyl)-N-(1-(4-(5-(6-(4-((1-(4-(2,4-dioxotetrahydropyrimidin-1(2H)-yl)phenyl)piperidin-4-yl)methyl)piperazin-1-yl)pyridin-3-yl)-1H-pyrazolo[3,4-b]pyridin-3-yl)-2-(hydroxymethyl)phenyl)ethyl)-1,2,4-oxadiazole-3-carboxamide

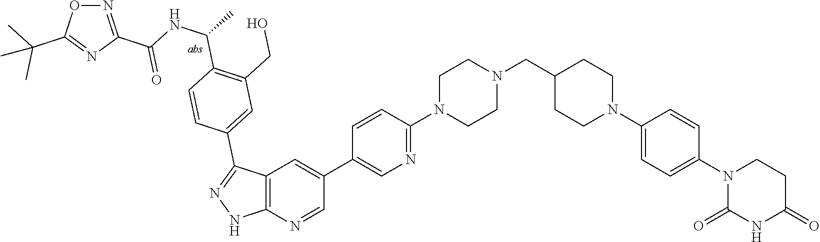 [SMILES: C[C@@H](NC(=O)c1noc(C(C)(C)C)n1)c1ccc(-c2n[nH]c3ncc(-c4ccc(N5CCN(CC6CCN(c7ccc(N8CCC(=O)NC8=O)cc7)CC6)CC5)nc4)cc23)cc1CO]

The titled compound was synthesized in the procedures similar to Example 1. ¹H NMR (400 MHz, DMSO) δ_{H} 13.82 (s, 1H), 10.27 (s, 1H), 9.54 (s, 1H), 8.84 (s, 1H), 8.61 (d, J=24.0 Hz, 2H), 8.12-7.82 (m, 3H), 7.65 (s, 1H), 7.13 (s, 2H), 6.94 (s, 3H), 5.34 (s, 2H), 4.80 (d, J=24.0 Hz, 2H), 3.69 (s, 4H), 3.56 (s, 4H), 2.67 (s, 4H), 2.43-2.35 (m, 1H), 2.22 (s, 2H), 1.94-1.64 (m, 4H), 1.52 (s, 3H), 1.42 (s, 9H), 1.23 (s, 3H); [M+H]⁺=867.8.

### Example 179: (R)-5-(tert-butyl)-N-(1-(4-(5-(6-(4-((1-(4-(2,4-dioxotetrahydropyrimidin-1(2H)-yl)-3-methylphenyl)piperidin-4-yl)methyl)piperazin-1-yl)pyridin-3-yl)-1H-pyrazolo[3,4-b]pyridin-3-yl)-2-(hydroxymethyl)phenyl)ethyl)-1,2,4-oxadiazole-3-carboxamide

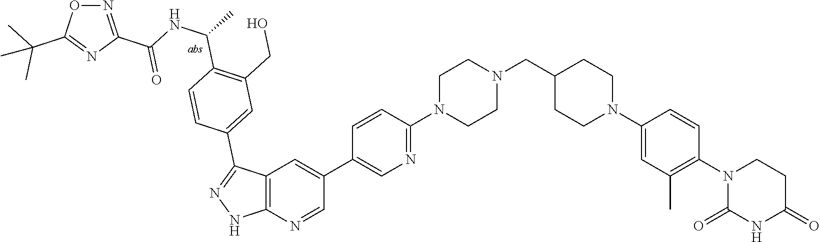 [SMILES: Cc1cc(N2CCC(CN3CCN(c4ccc(-c5cnc6[nH]nc(-c7ccc([C@@H](C)NC(=O)c8noc(C(C)(C)C)n8)c(CO)c7)c6c5)cn4)CC3)CC2)ccc1N1CCC(=O)NC1=O]

The titled compound was synthesized in the procedures similar to Example 1. ¹H NMR (400 MHz, DMSO) δ_{H} 13.82 (s, 1H), 10.25 (s, 1H), 9.54 (d, J=8.0 Hz, 1H), 8.84 (s, 1H), 8.67-8.55 (m, 2H), 8.13-7.91 (m, 3H), 7.66 (d, J=8.0 Hz, 1H), 7.05 (d, J=8.0 Hz, 1H), 6.97 (d, J=8.0 Hz, 1H), 6.86-6.72 (m, 2H), 5.43-5.29 (m, 2H), 4.89-4.72 (m, 2H), 3.79-3.61 (m, 4H), 3.57 (s, 3H), 3.52-3.41 (m, 2H), 3.12 (s, 1H), 2.80-2.59 (m, 5H), 2.48-2.44 (m, 2H), 2.22 (s, 2H), 2.12 (s, 3H), 1.87-1.64 (m, 3H), 1.53 (d, J=8.0 Hz, 3H), 1.42 (s, 9H); [M+H]⁺=881.7.

### Example 180: 5-(tert-butyl)-N-((1R)-1-(4-(5-(4-(4-(1-(4-((2,6-dioxopiperidin-3-yl)amino)phenyl)piperidin-4-yl)piperazin-1-yl)phenyl)-1H-pyrazolo[3,4-b]pyridin-3-yl)-2-methylphenyl)ethyl)-1,2,4-oxadiazole-3-carboxamide

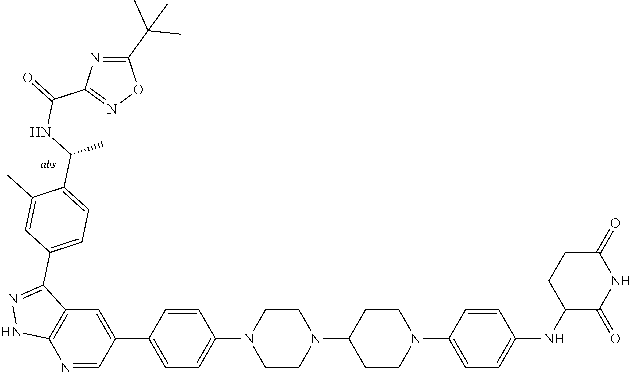 [SMILES: Cc1cc(-c2n[nH]c3ncc(-c4ccc(N5CCN(C6CCN(c7ccc(NC8CCC(=O)NC8=O)cc7)CC6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.81 (s, 1H), 10.78 (s, 1H), 9.92 (d, J=8.0 Hz, 1H), 8.83 (s, 1H), 8.60 (s, 1H), 7.92 (d, J=8.0 Hz, 1H), 7.86 (s, 1H), 7.74 (s, 2H), 7.62 (d, J=8.0 Hz, 1H), 7.13 (s, 2H), 6.80 (d, J=4.0 Hz, 2H), 6.62 (d, J=8.0 Hz, 2H), 5.45 (s, 1H), 5.35 (t, J=8.0 Hz, 1H), 4.21 (s, 1H), 4.14-3.75 (m, 4H), 3.61-3.46 (m, 7H), 3.03 (s, 3H), 2.80-2.65 (m, 2H), 2.64-2.52 (m, 5H), 2.17-2.06 (m, 2H), 1.93-1.80 (m, 2H), 1.54 (d, J=8.0 Hz, 3H), 1.37 (s, 9H); [M+H]⁺=850.7.

### Example 181 and 182: 5-(tert-butyl)-N—((R)-1-(4-(5-(5-(4-(4-((R)-2,6-dioxopiperidin-3-yl)-3-fluorophenethyl)piperazin-1-yl)pyridin-2-yl)-1H-pyrazolo[3,4-b]pyridin-3-yl)-2-methylphenyl)ethyl)-1,2,4-oxadiazole-3-carboxamide and 5-(tert-butyl)-N—((R)-1-(4-(5-(5-(4-(4-((S)-2,6-dioxopiperidin-3-yl)-3-fluorophenethyl)piperazin-1-yl)pyridin-2-yl)-1H-pyrazolo[3,4-b]pyridin-3-yl)-2-methylphenyl)ethyl)-1,2,4-oxadiazole-3-carboxamide

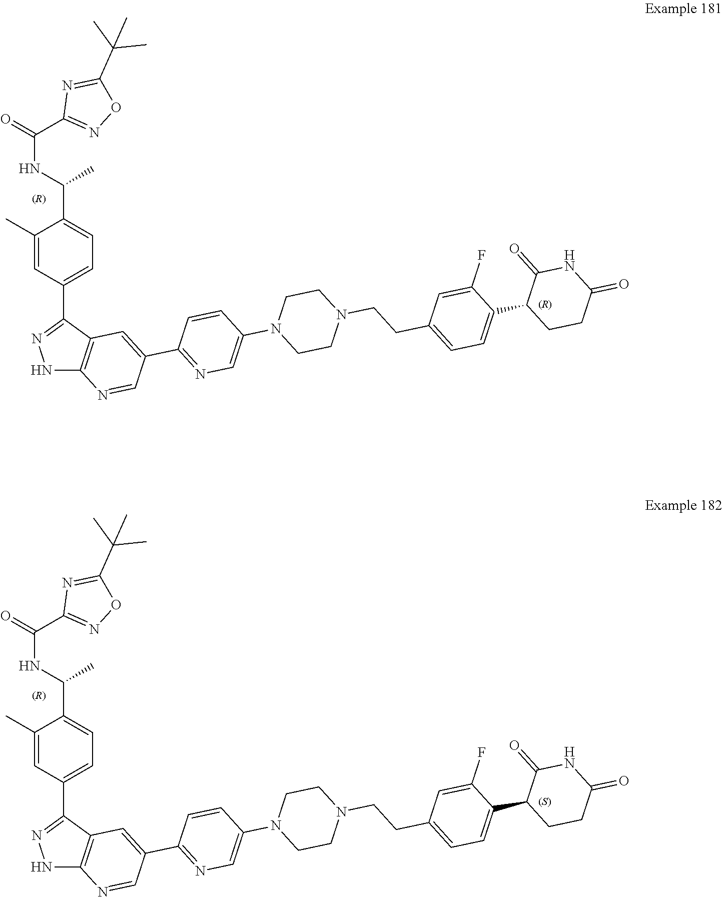

The Example 146 (43 mg) was separated by Prep-Chiral-HPLC with following conditions: Column: CHIRALPAK IA, 2*25 cm, 5 μm; Mobile Phase A: DCM, Mobile Phase B: EtOH; Flow rate: 16 mL/min; Gradient: 70% B to 70% B in 10 min; Detector: 220/254 nm; RT1: 6.404 min; RT2: 7.999 min; Sample Solvent: EtOH:DCM=3:1; Injection Volume: 0.8 mL; Number Of Runs: 8. This resulted in Example 181 (RT1: 1.614 min) (11.8 mg, 27.4%) and Example 182 (RT2: 2.090 min) (11.1 mg, 25.8%). Example 181: ¹H NMR (400 MHz, CDCl₃) δ_{H} 11.27 (s, 1H), 9.19 (s, 1H), 8.89 (s, 1H), 8.44 (s, 1H), 8.22 (s, 1H), 7.91-7.87 (m, 2H), 7.70 (d, J=8.0 Hz, 1H), 7.54 (d, J=8.0 Hz, 1H), 7.31 (d, J=8.0 Hz, 1H), 7.16-7.15 (m, 2H), 7.10-7.00 (m, 2H), 5.60 (s, 1H), 3.91-3.90 (m, 1H), 3.35 (s, 4H), 2.86-2.73 (m, 2H), 2.85-2.72 (m, 8H), 2.60 (s, 3H), 2.31-2.21 (m, 2H), 1.70-1.68 (m, 3H), 1.46 (s, 9H); [M+H]⁺=799.8. Example 182: ¹H NMR (400 MHz, CDCl₃) δ_{H} 11.46 (s, 1H), 9.19 (s, 1H), 8.89 (s, 1H), 8.44 (s, 1H), 8.22 (s, 1H), 7.91-7.87 (m, 2H), 7.70 (d, J=8.0 Hz, 1H), 7.54 (d, J=8.0 Hz, 1H), 7.31 (d, J=8.0 Hz, 1H), 7.14-7.15 (m, 2H), 7.03-7.00 (m, 2H), 5.61 (s, 1H), 3.93-3.89 (m, 1H), 3.35 (s, 4H), 2.87-2.74 (m, 2H), 2.85-2.72 (m, 8H), 2.60 (s, 3H), 2.31-2.21 (m, 2H), 1.70-1.68 (m, 3H), 1.46 (s, 9H); [M+H]⁺=799.8.

### Example 183: 5-(tert-butyl)-N-((1R)-1-(4-(5-(4-(4-((4-(2,6-dioxopiperidin-3-yl)phenoxy)methyl)piperidin-1-yl)phenyl)-1H-pyrazolo[3,4-b]pyridin-3-yl)-2-methylphenyl)ethyl)-1,2,4-oxadiazole-3-carboxamide

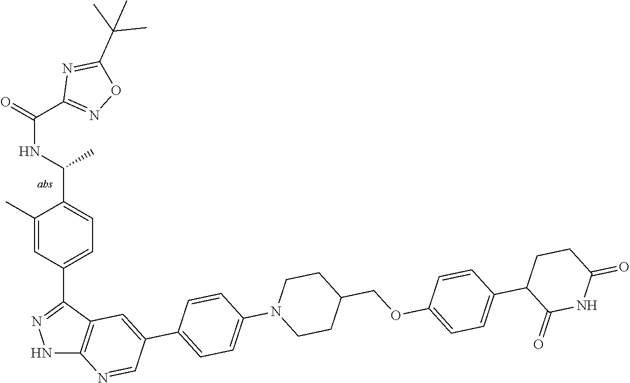 [SMILES: Cc1cc(-c2n[nH]c3ncc(-c4ccc(N5CCC(COc6ccc(C7CCC(=O)NC7=O)cc6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.79 (s, 1H), 10.81 (s, 1H), 9.50 (d, J=7.2 Hz, 1H), 8.82 (s, 1H), 8.58 (s, 1H), 7.92 (d, J=8.0 Hz, 1H), 7.85 (s, 1H), 7.68 (d, J=8.4 Hz, 2H), 7.60 (d, J=8.0 Hz, 1H), 7.13 (d, J=8.4 Hz, 2H), 7.08 (d, J=7.2 Hz, 2H), 6.91 (d, J=8.0 Hz, 2H), 5.36 (s, 1H), 3.96-3.74 (m, 5H), 2.77 (t, J=11.6 Hz, 2H), 2.64 (brs, 1H), 2.50 (s, 3H), 2.19-2.15 (m, 1H), 2.08-1.82 (m, 4H), 1.51 (d, J=6.4 Hz, 3H), 1.42 (s, 12H); [M+H]⁺=781.7.

### Example 184: (R)—N-(1-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylphenyl)ethyl)-5-(1-(hydroxymethyl)cyclopropyl)-1,2,4-oxadiazole-3-carboxamide

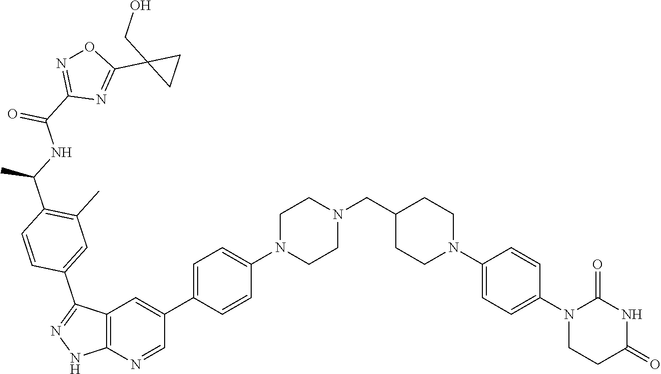 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1[C@@H](C)NC(=O)c1noc(C2(CO)CC2)n1]

The titled compound was synthesized in the procedures similar to Example 1. ¹H NMR (400 MHz, DMSO) δ_{H} 13.78 (s, 1H), 10.27 (s, 1H), 9.45 (d, J=8.0 Hz, 1H), 8.82 (s, 1H), 8.58 (s, 1H), 7.97-7.88 (m, 1H), 7.84 (s, 1H), 7.69 (d, J=8.4 Hz, 2H), 7.59 (d, J=8.0 Hz, 1H), 7.14 (d, J=8.4 Hz, 2H), 7.07 (d, J=8.4 Hz, 2H), 6.93 (d, J=8.4 Hz, 2H), 5.42-5.26 (m, 1H), 5.14-5.05 (m, 1H), 3.84-3.76 (m, 2H), 3.74-3.64 (m, 4H), 3.26-3.16 (m, 4H), 2.77-2.60 (m, 5H), 2.57-2.52 (m, 5H), 2.30-2.18 (m, 2H), 1.93-1.88 (m, 1H), 1.88-1.79 (m, 2H), 1.78-1.68 (m, 1H), 1.54-1.46 (m, 3H), 1.36-1.30 (m, 2H), 1.30-1.17 (m, 4H); [M+H]⁺=865.0.

### Example 185: (R)—N-(1-(4-(5-(4-(4-((1-(4-(2,4-dioxotetrahydropyrimidin-1(2H)-yl)-3-methylphenyl)piperidin-4-yl)methyl)piperazin-1-yl)phenyl)-1H-pyrazolo[3,4-b]pyridin-3-yl)-2-methylphenyl)ethyl)-5-(1-(hydroxymethyl)cyclopropyl)-1,2,4-oxadiazole-3-carboxamide

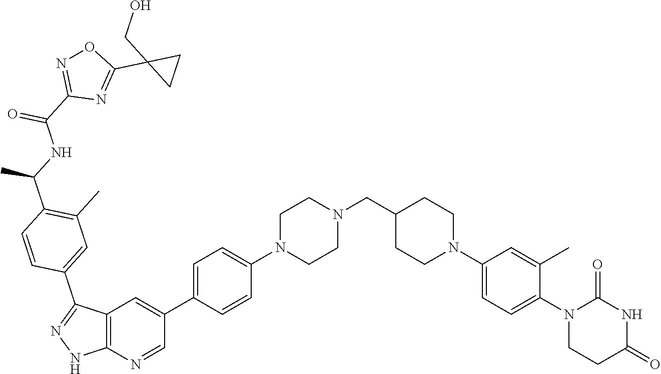 [SMILES: Cc1cc(-c2n[nH]c3ncc(-c4ccc(N5CCN(CC6CCN(c7ccc(N8CCC(=O)NC8=O)c(C)c7)CC6)CC5)cc4)cc23)ccc1[C@@H](C)NC(=O)c1noc(C2(CO)CC2)n1]

The titled compound was synthesized in the procedures similar to Example 1. ¹H NMR (400 MHz, DMSO) δ_{H} 13.82 (s, 1H), 10.26 (s, 1H), 9.47 (d, J=7.6 Hz, 1H), 8.82 (s, 1H), 8.58 (s, 1H), 7.91 (d, J=7.6 Hz, 1H), 7.85 (s, 1H), 7.69 (d, J=8.0 Hz, 2H), 7.60 (d, J=8.0 Hz, 1H), 7.14-6.98 (m, 3H), 6.87-6.71 (m, 2H), 5.44-5.30 (m, 1H), 3.85-3.77 (m, 2H), 3.75-3.64 (m, 3H), 3.56-3.41 (m, 2H), 3.25-3.17 (m, 4H), 2.73-2.63 (m, 4H), 2.58-2.52 (m, 4H), 2.29-2.20 (m, 2H), 2.12 (s, 3H), 1.88-1.65 (m, 6H), 1.51 (d, J=6.0 Hz, 3H), 1.38-1.30 (m, 2H), 1.29-1.20 (m, 4H); [M+H]⁺=879.0.

### Example 186: 5-(tert-butyl)-N-((1R)-1-(4-(5-(4-(4-(2-(4-(2,6-dioxopiperidin-3-yl)-1H-pyrazol-1-yl)ethyl)piperazin-1-yl)phenyl)-1H-pyrazolo[3,4-b]pyridin-3-yl)-2-methylphenyl)ethyl)-1,2,4-oxadiazole-3-carboxamide

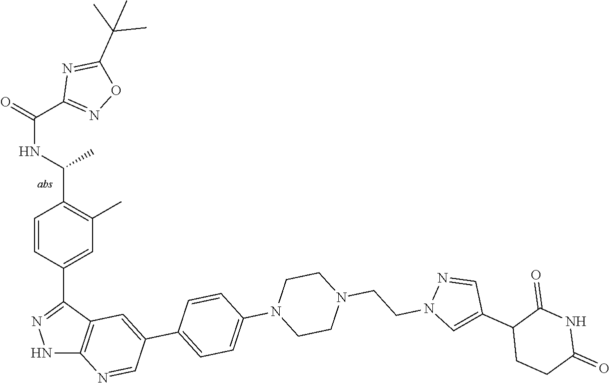 [SMILES: Cc1cc(-c2n[nH]c3ncc(-c4ccc(N5CCN(CCn6cc(C7CCC(=O)NC7=O)cn6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.77 (s, 1H), 10.75 (s, 1H), 9.48 (d, J=7.6 Hz, 1H), 8.82 (s, 1H), 8.58 (s, 1H), 7.91 (d, J=7.2 Hz, 1H), 7.85 (s, 1H), 7.72-7.66 (m, 3H), 7.60 (d, J=8.0 Hz, 1H), 7.40-7.35 (m, 1H), 7.06 (d, J=8.0 Hz, 2H), 5.42-5.30 (m, 1H), 5.07 (d, J=7.2 Hz, 1H), 4.28-4.20 (m, 2H), 3.94-3.72 (m, 1H), 3.19 (s, 3H), 2.80-2.73 (m, 2H), 2.62-2.55 (m, 4H), 2.20-2.00 (m, 2H), 1.51 (d, J=6.8 Hz, 3H), 1.42 (s, 9H); [M+H]⁺=770.9.

### Example 187: (R)-5-(tert-butyl)-N-(1-(4-(5-(4-(4-((4-(2,4-dioxotetrahydropyrimidin-1(2H)-yl)phenoxy)methyl)piperidin-1-yl)phenyl)-1H-pyrazolo[3,4-b]pyridin-3-yl)-2-methylphenyl)ethyl)-1,2,4-oxadiazole-3-carboxamide

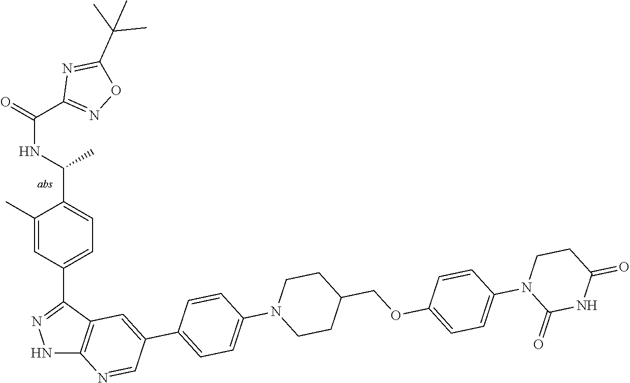 [SMILES: Cc1cc(-c2n[nH]c3ncc(-c4ccc(N5CCC(COc6ccc(N7CCC(=O)NC7=O)cc6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.79 (s, 1H), 10.81 (s, 1H), 9.50 (d, J=7.2 Hz, 1H), 8.82 (s, 1H), 8.58 (s, 1H), 7.92 (d, J=8.0 Hz, 1H), 7.85 (s, 1H), 7.68 (d, J=8.4 Hz, 2H), 7.60 (d, J=8.0 Hz, 1H), 7.13 (d, J=8.4 Hz, 2H), 7.08 (d, J=7.2 Hz, 2H), 6.91 (d, J=8.0 Hz, 2H), 5.36 (s, 1H), 3.96-3.74 (m, 5H), 2.77 (t, J=11.6 Hz, 2H), 2.64 (brs, 1H), 2.50 (s, 3H), 2.19-2.15 (m, 1H), 2.08-1.82 (m, 4H), 1.51 (d, J=6.4 Hz, 3H), 1.42 (s, 12H); [M+H]⁺=782.9.

### Example 188: 3-(tert-butyl)-N-((1R)-1-(4-(5-(4-(4-(2-(4-(2,6-dioxopiperidin-3-yl)phenyl)-2,2-difluoroethyl)piperazin-1-yl)phenyl)-1H-pyrazolo[3,4-b]pyridin-3-yl)-2-methylphenyl)ethyl)-1,2,4-oxadiazole-5-carboxamide

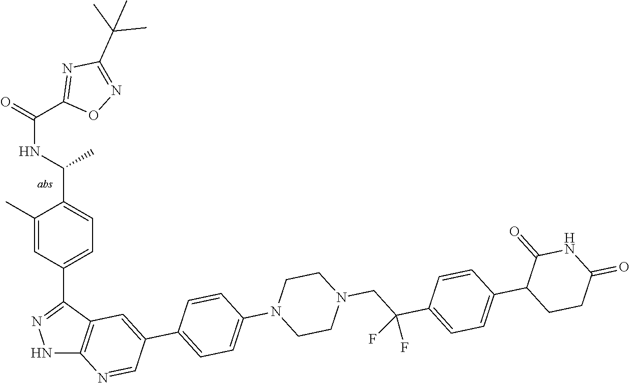 [SMILES: Cc1cc(-c2n[nH]c3ncc(-c4ccc(N5CCN(CC(F)(F)c6ccc(C7CCC(=O)NC7=O)cc6)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.78 (s, 1H), 10.90 (s, 1H), 9.92 (d, J=6.3 Hz, 1H), 8.81 (s, 1H), 8.58 (s, 1H), 7.96-7.83 (m, 2H), 7.72-7.52 (m, 5H), 7.36 (d, J=7.0 Hz, 2H), 7.04 (d, J=8.1 Hz, 2H), 5.40-5.31 (m, 1H), 4.01-3.91 (m, 1H), 3.24-3.08 (m, 7H), 2.78-2.53 (m, 8H), 2.31-2.18 (m, 1H), 2.11-2.01 (m, 1H), 1.53 (d, J=5.9 Hz, 3H), 1.36 (s, 9H); [M+H]⁺=816.8.

### Example 189: 5-(tert-butyl)-N-((1R)-1-(4-(5-(5-(5-(2-(5-(2,6-dioxopiperidin-3-yl)-6-methylpyridin-2-yl)ethyl)hexahydropyrrolo[3,4-c]pyrrol-2(1H)-yl)pyridin-2-yl)-1H-pyrazolo[3,4-b]pyridin-3-yl)-2-methylphenyl)ethyl)-1,2,4-oxadiazole-3-carboxamide

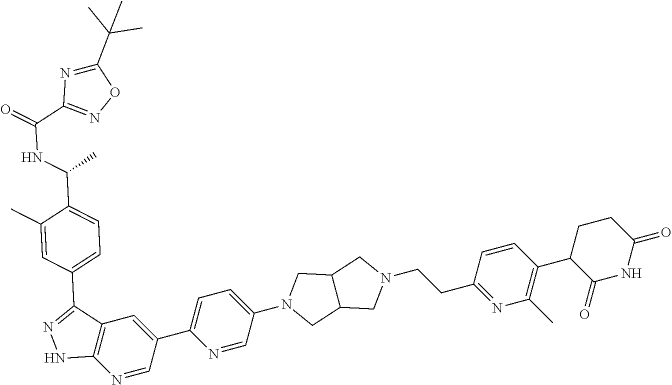 [SMILES: Cc1cc(-c2n[nH]c3ncc(-c4ccc(N5CC6CN(CCc7ccc(C8CCC(=O)NC8=O)c(C)n7)CC6C5)cn4)cc23)ccc1[C@@H](C)NC(=O)c1noc(C(C)(C)C)n1]

The titled compound was synthesized in the procedures similar to Example 1. ¹H NMR (400 MHz, CD₃OD) δ_{H} 9.04 (s, 1H), 8.92 (s, 1H), 8.20 (s, 1H), 8.05 (s, 1H), 7.87 (s, 2H), 7.71 (s, 1H), 7.61 (s, 2H), 7.30-7.26 (m, 1H), 5.53-5.48 (m, 1H), 5.42-5.29 (m, 1H), 4.12-4.10 (m, 3H), 3.76-3.72 (m, 4H), 3.65-3.60 (m, 4H), 3.46-3.42 (m, 4H), 2.77-2.67 (m, 3H), 2.58 (s, 3H), 2.46 (s, 3H), 2.28-2.26 (m, 2H), 2.02-2.00 (m, 1H), 1.52 (d, J=4.0 Hz, 3H), 1.42 (s, 9H); [M+H]⁺=822.6.

### Example 190: (R)-3-(tert-butyl)-N-(1-(4-(5-(4-(4-((1-(4-(2,4-dioxotetrahydropyrimidin-1(2H)-yl)-3-methylphenyl)piperidin-4-yl)methyl)piperazin-1-yl)phenyl)-1H-pyrazolo[3,4-b]pyridin-3-yl)-5-fluoro-2-(hydroxymethyl)phenyl)ethyl)-1,2,4-oxadiazole-5-carboxamide

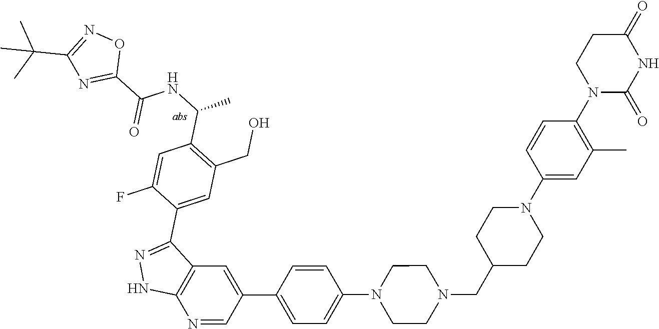 [SMILES: Cc1cc(N2CCC(CN3CCN(c4ccc(-c5cnc6[nH]nc(-c7cc(CO)c([C@@H](C)NC(=O)c8nc(C(C)(C)C)no8)cc7F)c6c5)cc4)CC3)CC2)ccc1N1CCC(=O)NC1=O]

The titled compound was synthesized in the procedures similar to Example 1. ¹H NMR (400 MHz, DMSO) δ_{H} 14.00 (s, 1H), 10.24 (s, 1H), 9.93 (d, J=8.0 Hz, 1H), 8.85 (s, 1H), 8.32 (s, 1H), 7.89 (d, J=8.0 Hz, 1H), 7.71-7.53 (m, 3H), 7.13-6.99 (m, 3H), 6.95-6.66 (m, 2H), 5.46-5.33 (m, 2H), 4.88-4.64 (m, 2H), 3.75-3.60 (m, 3H), 3.55-3.38 (m, 2H), 3.21 (s, 5H), 2.78-2.62 (m, 5H), 2.60-2.52 (m, 3H), 2.20 (s, 2H), 2.12 (s, 3H), 1.82 (d, J=8.0 Hz, 2H), 1.73 (s, 1H), 1.56 (d, J=8.0 Hz, 3H), 1.37 (s, 9H); [M+H]⁺=898.8.

### Example 191: 5-(tert-butyl)-N-((1R)-1-(4-(5-(4-(4-((1-(4-(2,6-dioxopiperidin-3-yl)phenyl)pyrrolidin-3-yl)methyl)piperazin-1-yl)phenyl)-1H-pyrazolo[3,4-b]pyridin-3-yl)-2-methylphenyl)ethyl)-1,2,4-oxadiazole-3-carboxamide

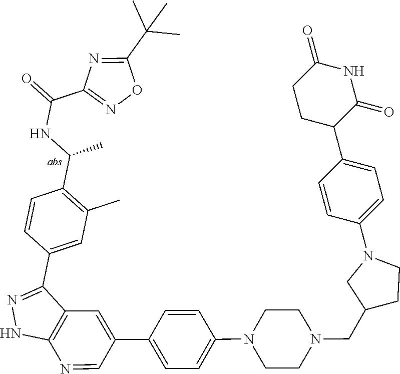 [SMILES: Cc1cc(-c2n[nH]c3ncc(-c4ccc(N5CCN(CC6CCN(c7ccc(C8CCC(=O)NC8=O)cc7)C6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.78 (s, 1H), 10.75 (s, 1H), 9.48 (d, J=8.0 Hz, 1H), 8.82 (s, 1H), 8.59 (s, 1H), 7.88-7.86 (m, 1H), 7.85 (s, 1H), 7.68 (d, J=8.0 Hz, 2H), 7.58 (d, J=8.0 Hz, 1H), 7.10-6.98 (m, 4H), 6.49 (d, J=8.0 Hz, 2H), 5.38-5.34 (m, 1H), 3.70-3.68 (m, 1H), 3.36-3.34 (m, 5H), 3.33-3.30 (m, 6H), 3.30-3.28 (m, 4H), 3.02-2.98 (m, 2H), 2.50 (s, 3H), 2.50-2.46 (m, 4H), 2.14-1.96 (m, 3H), 1.62-1.58 (m, 1H), 1.52 (d, J=4.0 Hz, 3H), 1.42 (s, 9H); [M+H]⁺=836.0.

### Example 192: 3-(tert-butyl)-N-((1R)-1-(4-(5-(4-(4-(2-(5-(2,6-dioxopiperidin-3-yl)pyridin-2-yl)-2,2-difluoroethyl)piperazin-1-yl)phenyl)-1H-pyrazolo[3,4-b]pyridin-3-yl)-2-methylphenyl)ethyl)-1,2,4-oxadiazole-5-carboxamide

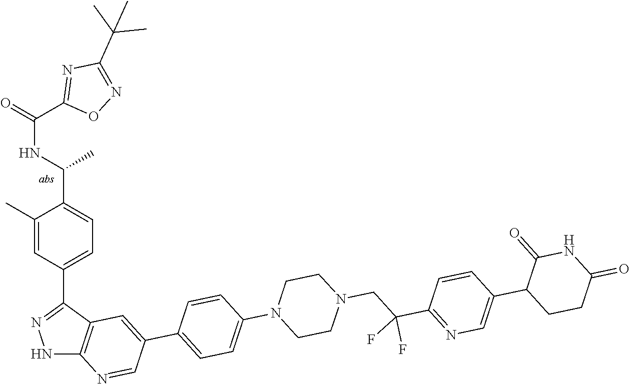 [SMILES: Cc1cc(-c2n[nH]c3ncc(-c4ccc(N5CCN(CC(F)(F)c6ccc(C7CCC(=O)NC7=O)cn6)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.76 (s, 1H), 10.96 (s, 1H), 9.91 (d, J=7.6 Hz, 1H), 8.80 (s, 1H), 8.58 (d, J=4.1 Hz, 2H), 7.96-7.81 (m, 3H), 7.76-7.56 (m, 4H), 7.03 (d, J=8.4 Hz, 2H), 5.43-5.29 (m, 1H), 4.11-4.02 (m, 1H), 3.19-2.98 (m, 6H), 2.84-2.55 (m, 10H), 2.40-2.24 (m, 2H), 2.12-2.01 (m, 1H), 1.51 (d, J=6.6 Hz, 3H), 1.42 (s, 9H); [M+H]⁺=817.6.

### Example 193: 5-(tert-butyl)-N-((1R)-1-(4-(5-(4-(4-(2-(5-(2,6-dioxopiperidin-3-yl)pyridin-2-yl)-2,2-difluoroethyl)piperazin-1-yl)phenyl)-1H-pyrazolo[3,4-b]pyridin-3-yl)-2-methylphenyl)ethyl)-1,2,4-oxadiazole-3-carboxamide

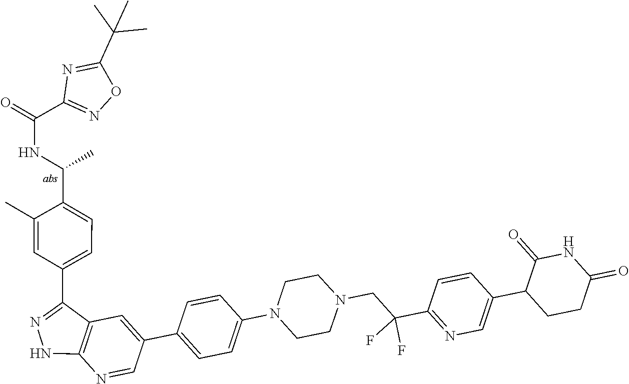 [SMILES: Cc1cc(-c2n[nH]c3ncc(-c4ccc(N5CCN(CC(F)(F)c6ccc(C7CCC(=O)NC7=O)cn6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.76 (s, 1H), 10.96 (s, 1H), 9.47 (d, J=7.6 Hz, 1H), 8.80 (s, 1H), 8.58 (d, J=4.1 Hz, 2H), 7.96-7.81 (m, 3H), 7.76-7.56 (m, 4H), 7.03 (d, J=8.4 Hz, 2H), 5.43-5.29 (m, 1H), 4.11-4.02 (m, 1H), 3.19-2.98 (m, 6H), 2.84-2.55 (m, 10H), 2.40-2.24 (m, 2H), 2.12-2.01 (m, 1H), 1.51 (d, J=6.6 Hz, 3H), 1.42 (s, 9H); [M+H]⁺=817.8.

### Example 194: (R)-5-(tert-butyl)-N-(1-(4-(5-(4-(4-(2-(5-(2,4-dioxotetrahydropyrimidin-1(2H)-yl)pyridin-2-yl)ethyl)piperazin-1-yl)phenyl)-1H-pyrazolo[3,4-b]pyridin-3-yl)-2-methylphenyl)ethyl)-1,2,4-oxadiazole-3-carboxamide

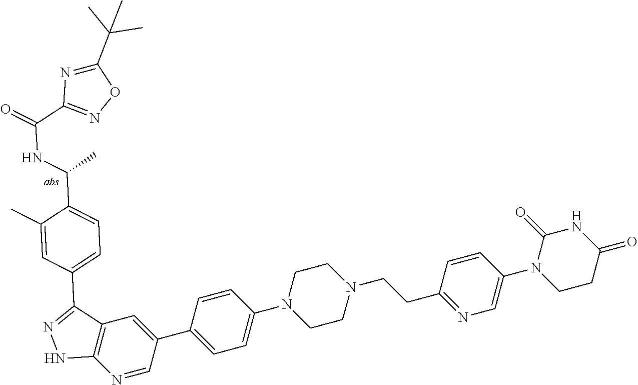 [SMILES: Cc1cc(-c2n[nH]c3ncc(-c4ccc(N5CCN(CCc6ccc(N7CCC(=O)NC7=O)cn6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.76 (s, 1H), 10.47 (s, 1H), 9.47 (d, J=8.0 Hz, 1H), 8.82 (s, 1H), 8.58 (s, 1H), 8.48 (s, 1H), 7.91 (d, J=7.6 Hz, 1H), 7.85 (s, 1H), 7.68 (d, J=8.4 Hz, 3H), 7.60 (d, J=8.0 Hz, 1H), 7.37 (d, J=8.8 Hz, 1H), 7.07 (d, J=8.4 Hz, 2H), 5.41-5.29 (m, 1H), 3.82 (t, J=6.8 Hz, 2H), 3.22 (s, 4H), 2.95 (s, 2H), 2.74 (t, J=6.4 Hz, 4H), 2.63 (s, 4H), 2.50 (s, 3H), 1.51 (d, J=6.8 Hz, 3H), 1.42 (s, 9H); [M+H]⁺=782.7.

### Example 195: 3-(tert-butyl)-N-((1R)-1-(4-(5-(4-(4-(2-(5-(2,6-dioxopiperidin-3-yl)-6-methoxypyridin-2-yl)ethyl)piperazin-1-yl)phenyl)-1H-pyrazolo[3,4-b]pyridin-3-yl)-2-methylphenyl)ethyl)-1,2,4-oxadiazole-5-carboxamide

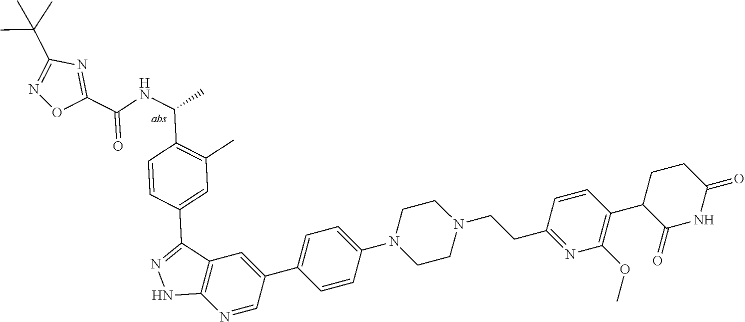 [SMILES: COc1nc(CCN2CCN(c3ccc(-c4cnc5[nH]nc(-c6ccc([C@@H](C)NC(=O)c7nc(C(C)(C)C)no7)c(C)c6)c5c4)cc3)CC2)ccc1C1CCC(=O)NC1=O]

The titled compound was synthesized in the procedures similar to Example 1. ¹H NMR (400 MHz, DMSO) δ_{H} 13.77 (s, 1H), 10.79 (s, 1H), 9.89 (d, J=7.8 Hz, 1H), 8.82 (s, 1H), 8.58 (s, 1H), 7.96-7.82 (m, 2H), 7.75-7.58 (m, 3H), 7.47 (d, J=6.9 Hz, 1H), 7.07 (d, J=8.3 Hz, 2H), 6.89 (d, J=6.1 Hz, 1H), 5.40-5.29 (m, 1H), 3.95-3.80 (m, 4H), 3.67-3.57 (m, 1H), 3.20-3.10 (m, 6H), 2.90-2.54 (m, 10H), 2.30-2.16 (m, 1H), 1.92-1.88 (m, 1H), 1.54 (d, J=6.8 Hz, 3H), 1.36 (s, 9H); [M+H]⁺=811.8.

### Example 196: 3-(tert-butyl)-N-((1R)-1-(4-(5-(4-(4-(2-(4-(2,6-dioxopiperidin-3-yl)-3,5-dimethyl-1H-pyrazol-1-yl)ethyl)piperazin-1-yl)phenyl)-1H-pyrazolo[3,4-b]pyridin-3-yl)-2-methylphenyl)ethyl)-1,2,4-oxadiazole-5-carboxamide

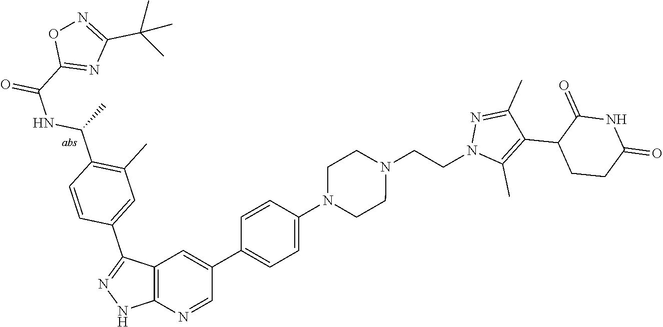 [SMILES: Cc1cc(-c2n[nH]c3ncc(-c4ccc(N5CCN(CCn6nc(C)c(C7CCC(=O)NC7=O)c6C)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.77 (s, 1H), 10.78 (s, 1H), 9.89 (d, J=7.6 Hz, 1H), 8.81 (s, 1H), 8.58 (s, 1H), 7.92 (d, J=7.6 Hz, 1H), 7.85 (s, 1H), 7.68 (d, J=8.4 Hz, 2H), 7.62 (d, J=8.4 Hz, 1H), 7.06 (d, J=8.0 Hz, 2H), 5.40-5.30 (m, 1H), 4.10-4.05 (m, 2H), 3.79-3.66 (m, 1H), 3.23-3.15 (m, 4H), 2.74-2.62 (m, 3H), 2.63-2.56 (m, 4H), 2.14 (s, 3H), 2.00-1.95 (m, 4H), 1.88-1.80 (m, 1H), 1.54 (d, J=6.8 Hz, 3H), 1.37 (s, 9H); [M+H]⁺=798.8.

### Example 197: 5-(tert-butyl)-N-((1R)-1-(4-(5-(6-(1-(3-(4-(2,6-dioxopiperidin-3-yl)phenyl)cyclobutyl)piperidin-4-yl)pyridin-3-yl)-1H-pyrazolo[3,4-b]pyridin-3-yl)-2-methylphenyl)ethyl)-1,2,4-oxadiazole-3-carboxamide

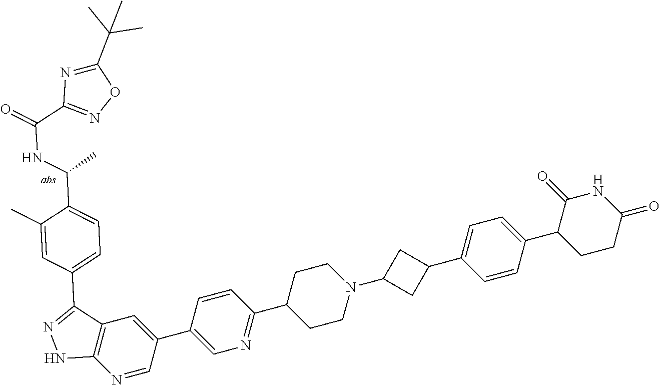 [SMILES: Cc1cc(-c2n[nH]c3ncc(-c4ccc(C5CCN(C6CC(c7ccc(C8CCC(=O)NC8=O)cc7)C6)CC5)nc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.90 (s, 1H), 10.82 (s, 1H), 9.48 (d, J=8.0 Hz, 1H), 8.98 (s, 1H), 8.90 (s, 1H), 8.79 (s, 1H), 8.20 (s, 1H), 7.95 (d, J=8.0 Hz, 1H), 7.88 (s, 1H), 7.60 (d, J=8.0 Hz, 1H), 7.48-7.18 (m, 5H), 5.36 (s, 1H), 3.83 (d, J=6.3 Hz, 1H), 3.06 (d, J=66.1 Hz, 5H), 2.67 (s, 4H), 2.50 (s, 3H), 2.27-1.71 (m, 10H), 1.51 (d, J=6.8 Hz, 3H), 1.42 (s, 9H); [M+H]⁺=806.8.

### Example 198: 5-(tert-butyl)-N-((1R)-1-(4-(5-(4-(4-((4-((2,6-dioxopiperidin-3-yl)oxy)phenoxy)methyl)piperidin-1-yl)phenyl)-1H-pyrazolo[3,4-b]pyridin-3-yl)-2-methylphenyl)ethyl)-1,2,4-oxadiazole-3-carboxamide

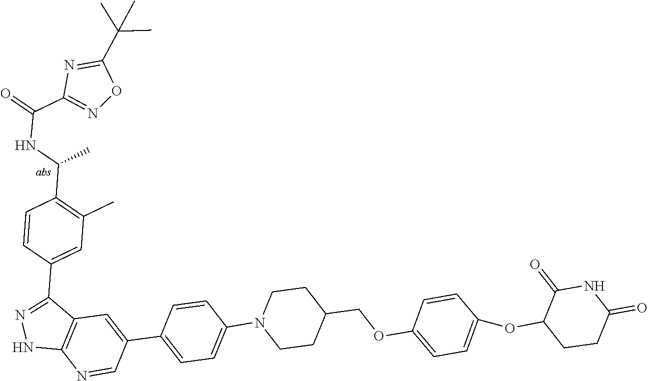 [SMILES: Cc1cc(-c2n[nH]c3ncc(-c4ccc(N5CCC(COc6ccc(OC7CCC(=O)NC7=O)cc6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.77 (s, 1H), 10.90 (s, 1H), 9.47 (d, J=8.0 Hz, 1H), 8.82 (s, 1H), 8.58 (s, 1H), 7.91 (d, J=7.6 Hz, 1H), 7.85 (s, 1H), 7.68 (d, J=8.4 Hz, 2H), 7.60 (d, J=8.0 Hz, 1H), 7.08 (d, J=8.4 Hz, 2H), 6.95 (d, J=9.2 Hz, 2H), 6.88 (d, J=9.2 Hz, 2H), 5.43-5.31 (m, 1H), 5.12-4.98 (m, 1H), 3.88-3.75 (m, 4H), 2.87-2.56 (m, 4H), 2.50 (s, 3H), 2.18-2.08 (m, 2H), 1.88 (d, J=13.2 Hz, 2H), 1.51 (d, J=6.8 Hz, 3H), 1.42 (s, 12H); [M+H]⁺=797.7.

### Example 199: 5-(tert-butyl)-N-((1R)-1-(4-(5-(6-(4-(4-(2,6-dioxopiperidin-3-yl)-3-fluorophenethyl)piperazin-1-yl)pyridin-3-yl)-1H-pyrazolo[3,4-b]pyridin-3-yl)-2-methylphenyl)ethyl)-1,2,4-oxadiazole-3-carboxamide

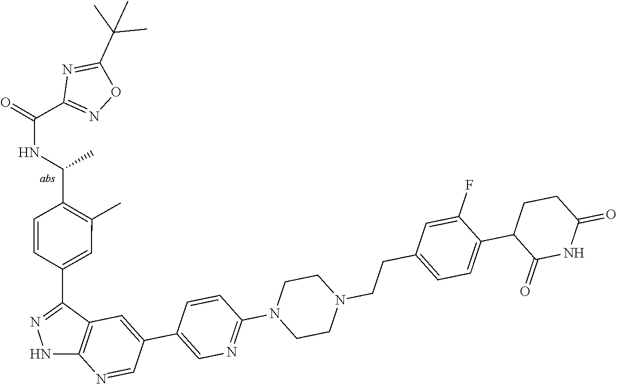 [SMILES: Cc1cc(-c2n[nH]c3ncc(-c4ccc(N5CCN(CCc6ccc(C7CCC(=O)NC7=O)c(F)c6)CC5)nc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.80 (s, 1H), 10.86 (s, 1H), 9.90 (d, J=8.0 Hz, 1H), 8.83 (s, 1H), 8.65 (s, 1H), 8.60 (s, 1H), 8.04 (d, J=8.0 Hz, 1H), 7.94 (d, J=8.0 Hz, 1H), 7.87 (s, 1H), 7.61 (d, J=8.0 Hz, 1H), 7.22 (t, J=8.0 Hz, 1H), 7.17-7.06 (m, 2H), 6.98 (d, J=8.0 Hz, 1H), 5.36 (t, J=8.0 Hz, 1H), 4.01 (dd, J=12.0, 4.0 Hz, 1H), 3.57 (s, 3H), 2.99 (s, 1H), 2.81 (s, 2H), 2.76-2.64 (m, 2H), 2.58 (s, 9H), 2.19 (dd, J=24.0, 12.0 Hz, 1H), 2.00 (s, 1H), 1.53 (d, J=8.0 Hz, 3H), 1.36 (s, 9H); [M+H]⁺=799.7.

### Example 200: 3-(tert-butyl)-N-((1R)-1-(4-(5-(4-(4-(4-(2,5-dioxopyrrolidin-3-yl)phenethyl)piperazin-1-yl)phenyl)-1H-pyrazolo[3,4-b]pyridin-3-yl)-2-methylphenyl)ethyl)-1,2,4-oxadiazole-5-carboxamide

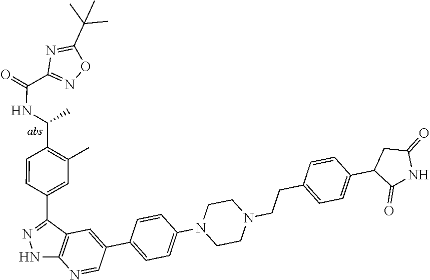 [SMILES: Cc1cc(-c2n[nH]c3ncc(-c4ccc(N5CCN(CCc6ccc(C7CC(=O)NC7=O)cc6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.76 (s, 1H), 11.30 (s, 1H), 9.88 (d, J=7.0 Hz, 1H), 8.81 (s, 1H), 8.58 (s, 1H), 7.92 (d, J=8.0 Hz, 1H), 7.85 (s, 1H), 7.68 (d, J=8.0 Hz, 2H), 7.62 (d, J=8.0 Hz, 1H), 7.22 (dd, J=16.0, 8.0 Hz, 4H), 7.07 (d, J=8.7 Hz, 2H), 5.46-5.26 (m, 1H), 4.15-4.05 (m, 1H), 3.22 (s, 5H), 3.11 (dd, J=16.0, 8.0 Hz, 2H), 2.78 (t, J=8.0 Hz, 2H), 2.74-2.66 (m, 1H), 2.66-2.56 (m, 6H), 1.54 (d, J=8.0 Hz, 3H), 1.37 (s, 9H); [M+H]⁺=766.7.

### Example 201: (R)-3-(tert-butyl)-N-(1-(4-(5-(4-(1-((1-(4-(2,4-dioxotetrahydropyrimidin-1(2H)-yl)phenyl)piperidin-4-yl)methyl)-4-hydroxypiperidin-4-yl)phenyl)-1H-pyrazolo[3,4-b]pyridin-3-yl)-2-methylphenyl)ethyl)-1,2,4-oxadiazole-5-carboxamide

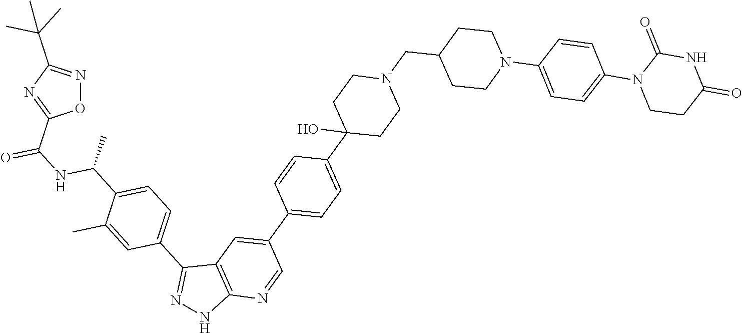 [SMILES: Cc1cc(-c2n[nH]c3ncc(-c4ccc(C5(O)CCN(CC6CCN(c7ccc(N8CCC(=O)NC8=O)cc7)CC6)CC5)cc4)cc23)ccc1[C@@H](C)NC(=O)c1nc(C(C)(C)C)no1]

### Step 1: tert-butyl 4-(4-bromophenyl)-4-hydroxypiperidine-1-carboxylate

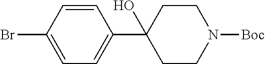 [SMILES: CC(C)(C)OC(=O)N1CCC(O)(c2ccc(Br)cc2)CC1]

To a solution of 1-bromo-4-iodobenzene (9.37 g, 33.2 mmol) in THF (120 mL) was added n-BuLi (2.5 M in hexane, 13.2 mL, 33.1 mmol) at −78° C. under a nitrogen atmosphere. The mixture was stirred at −78° C. for 2 h. Then, a solution of tert-butyl 4-oxopiperidine-1-carboxylate (6 g, 30.1 mmol) in THF (80 mL) was added dropwise and the mixture was stirred at −78° C. for 1 h. The mixture was quenched by water (150 mL) and extracted with EtOAc (3×100 mL). The combined organic layers were dried over Na₂SO₄, filtered and concentrated under vacuum. The residue was purified by silica gel column chromatography, eluted with EtOAc/PE (1:1) to afford the product (7.75 g, 72.4%). [M+H]⁺=356.1.

### Step 2: tert-butyl 4-(4-(3-(4-((R)-1-(3-(tert-butyl)-1,2,4-oxadiazole-5-carboxamido)ethyl)-3-methylphenyl)-1-(tetrahydro-2H-pyran-2-yl)-1H-pyrazolo[3,4-b]pyridin-5-yl)phenyl)-4-hydroxypiperidine-1-carboxylate

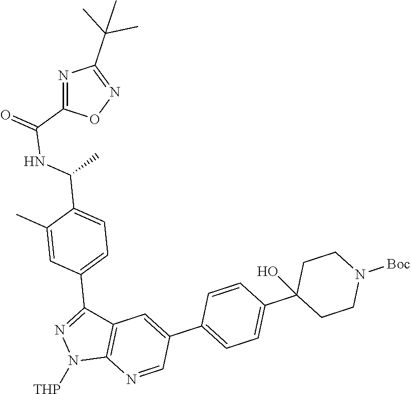

A mixture of 3-(tert-butyl)-N-((1R)-1-(2-methyl-4-(1-(tetrahydro-2H-pyran-2-yl)-5-(4,4,5,5-tetramethyl-1,3,2-dioxaborolan-2-yl)-1H-pyrazolo[3,4-b]pyridin-3-yl)phenyl)ethyl)-1,2,4-oxadiazole-5-carboxamide (307 mg, 0.5 mmol), tert-butyl 4-(4-bromophenyl)-4-hydroxypiperidine-1-carboxylate (213 mg, 0.6 mmol), Pd(dppf)Cl₂ (36.6 mg, 0.05 mmol) and K₂CO₃ (138 mg, 1 mmol) in dioxane (10 mL) and H₂O (2 mL) was stirred at 100° C. for 16 h under a nitrogen atmosphere. The mixture was concentrated under vacuum. The residue was purified by silica gel column chromatography, eluted with EtOAc/PE (1:1) to afford the product (220 mg, 57.6%). [M+H]⁺=764.4.

### Step 3: (R)-3-(tert-butyl)-N-(1-(4-(5-(4-(4-hydroxypiperidin-4-yl)phenyl)-1H-pyrazolo[3,4-b]pyridin-3-yl)-2-methylphenyl)ethyl)-1,2,4-oxadiazole-5-carboxamide hydrochloride

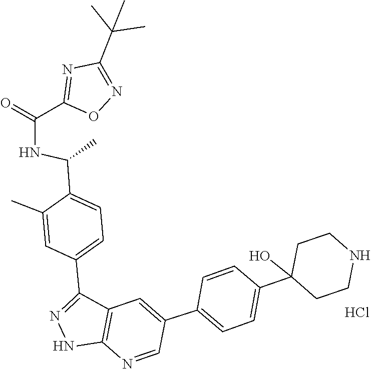 [SMILES: Cc1cc(-c2n[nH]c3ncc(-c4ccc(C5(O)CCNCC5)cc4)cc23)ccc1[C@@H](C)NC(=O)c1nc(C(C)(C)C)no1 ; Cl]

A mixture of tert-butyl 4-(4-(3-(4-((R)-1-(3-(tert-butyl)-1,2,4-oxadiazole-5-carboxamido)ethyl)-3-methylphenyl)-1-(tetrahydro-2H-pyran-2-yl)-1H-pyrazolo[3,4-b]pyridin-5-yl)phenyl)-4-hydroxypiperidine-1-carboxylate (50 mg) in 4 N HCl in dioxane (5 mL) was stirred at room temperature for 2.5 h. The mixture was concentrated under vacuum to afford the product (40 mg, crude), which was used in the next step directly. [M+H]⁺=580.3.

### Step 4: (R)-3-(tert-butyl)-N-(1-(4-(5-(4-(1-((1-(4-(2,4-dioxotetrahydropyrimidin-1(2H)-yl)phenyl)piperidin-4-yl)methyl)-4-hydroxypiperidin-4-yl)phenyl)-1H-pyrazolo[3,4-b]pyridin-3-yl)-2-methylphenyl)ethyl)-1,2,4-oxadiazole-5-carboxamide

A mixture of (R)-3-(tert-butyl)-N-(1-(4-(5-(4-(4-hydroxypiperidin-4-yl)phenyl)-1H-pyrazolo[3,4-b]pyridin-3-yl)-2-methylphenyl)ethyl)-1,2,4-oxadiazole-5-carboxamide hydrochloride (40 mg, 0.069 mmol), 1-(4-(2,4-dioxotetrahydropyrimidin-1(2H)-yl)phenyl)piperidine-4-carbaldehyde (25 mg, 0.083 mmol) and AcOH (0.2 mL) in MeOH (5 mL) and DCM (5 mL) was stirred at rt for 16 h. Then, STAB (29.3 mg, 0.14 mmol) was added to the mixture above and the mixture was stirred at room temperature for 6 h. The mixture was concentrated under vacuum. The residue was purified by Prep-TLC (MeOH/DCM=1:9) to afford the product (23.25 mg, 38.9%). ¹H NMR (400 MHz, DMSO) δ_{H} 13.87 (s, 1H), 10.27 (s, 1H), 9.92 (d, J=8.0 Hz, 1H), 8.88 (s, 1H), 8.70 (s, 1H), 7.94 (d, J=8.0 Hz, 1H), 7.67-7.58 (m, 3H), 7.62 (d, J=8.0 Hz, 3H), 7.15 (d, J=12.0 Hz, 2H), 6.97 (d, J=8.0 Hz, 2H), 5.58 (s, 1H), 5.40-5.31 (m, 1H), 3.80-3.66 (m, 4H), 3.58-3.46 (m, 2H), 3.42-3.34 (m, 2H), 3.31-3.22 (m, 2H), 3.18-3.08 (m, 2H), 2.78-2.65 (m, 4H), 2.54 (s, 3H), 2.15-2.02 (m, 1H), 1.98-1.89 (m, 2H), 1.88-1.80 (m, 2H), 1.54 (d, J=8.0 Hz, 3H), 1.37 (s, 11H); [M+H]⁺=865.4.

### Example 202: (R)-3-(tert-butyl)-N-(1-(4-(5-(4-(4-((1-(4-(2,4-dioxotetrahydropyrimidin-1(2H)-yl)phenyl)piperidin-4-yl)methyl)-1,4-diazepan-1-yl)phenyl)-1H-pyrazolo[3,4-b]pyridin-3-yl)-2-methylphenyl)ethyl)-1,2,4-oxadiazole-5-carboxamide

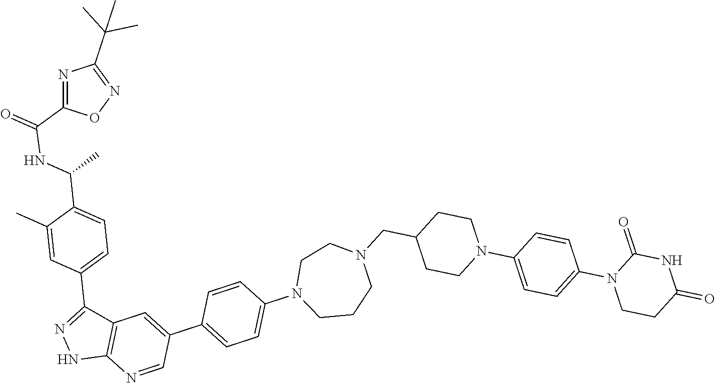 [SMILES: Cc1cc(-c2n[nH]c3ncc(-c4ccc(N5CCCN(CC6CCN(c7ccc(N8CCC(=O)NC8=O)cc7)CC6)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.74 (s, 1H), 10.25 (s, 1H), 9.90 (d, J=8.0 Hz, 1H), 8.81 (s, 1H), 8.54 (s, 1H), 7.91 (d, J=4.0 Hz, 1H), 7.85 (s, 1H), 7.68-7.59 (m, 3H), 7.15-7.06 (m, 2H), 6.93-6.80 (m, 4H), 5.40-5.31 (m, 1H), 3.72-3.62 (m, 5H), 3.60-3.50 (m, 5H), 2.81-2.72 (m, 2H), 2.71-2.60 (m, 5H), 2.53 (s, 3H), 2.37-2.30 (m, 2H), 1.90-1.85 (m, 1H), 1.80-1.72 (m, 2H), 1.53 (d, J=4.0 Hz, 3H), 1.36 (s, 9H), 1.25-1.13 (m, 3H); [M+H]⁺=864.5.

### Example 203: (R)-3-(tert-butyl)-N-(1-(4-(5-(4-(1-((1-(4-(2,4-dioxotetrahydropyrimidin-1(2H)-yl)phenyl)piperidin-4-yl)methyl)-4-fluoropiperidin-4-yl)phenyl)-1H-pyrazolo[3,4-b]pyridin-3-yl)-2-methylphenyl)ethyl)-1,2,4-oxadiazole-5-carboxamide

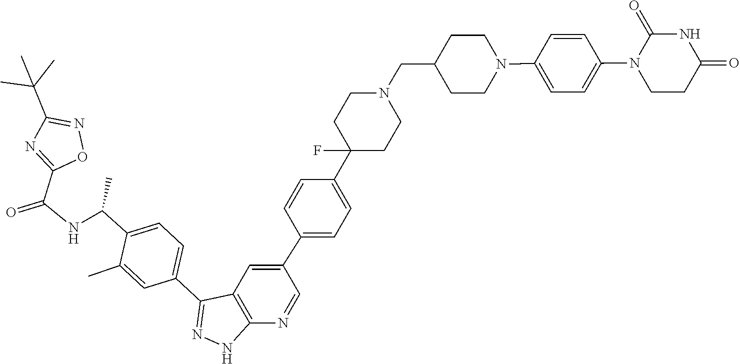 [SMILES: Cc1cc(-c2n[nH]c3ncc(-c4ccc(C5(F)CCN(CC6CCN(c7ccc(N8CCC(=O)NC8=O)cc7)CC6)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.86 (s, 1H), 10.26 (s, 1H), 9.91 (d, J=8.0 Hz, 1H), 8.88 (s, 1H), 8.71 (s, 1H), 7.94 (d, J=4.0 Hz, 1H), 7.90-7.81 (m, 3H), 7.65-7.54 (m, 3H), 7.14 (d, J=4.0 Hz, 2H), 6.94 (d, J=4.0 Hz, 2H), 5.40-5.31 (m, 1H), 3.74-3.65 (m, 4H), 2.89-2.79 (m, 2H), 2.73-2.63 (m, 4H), 2.57-2.52 (m, 2H), 2.51 (s, 3H), 2.36-2.23 (m, 4H), 2.02-1.91 (m, 2H), 1.89-1.80 (m, 2H), 1.79-1.68 (m, 1H), 1.54 (d, J=4.0 Hz, 3H), 1.37 (s, 9H), 1.30-1.19 (m, 2H); [M+H]⁺=867.4.

### Example 204: (R)-3-(tert-butyl)-N-(1-(4-(5-(5-(1-((1-(4-(2,4-dioxotetrahydropyrimidin-1(2H)-yl)phenyl)piperidin-4-yl)methyl)-4-hydroxypiperidin-4-yl)pyridin-2-yl)-1H-pyrazolo[3,4-b]pyridin-3-yl)-2-methylphenyl)ethyl)-1,2,4-oxadiazole-5-carboxamide

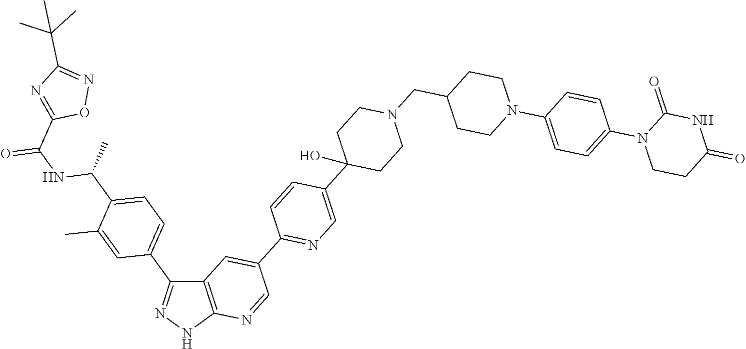 [SMILES: Cc1cc(-c2n[nH]c3ncc(-c4ccc(C5(O)CCN(CC6CCN(c7ccc(N8CCC(=O)NC8=O)cc7)CC6)CC5)cn4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.91 (s, 1H), 10.26 (s, 1H), 9.92 (d, J=8.0 Hz, 1H), 9.29 (s, 1H), 9.04 (s, 1H), 8.84 (s, 1H), 8.15 (d, J=8.0 Hz, 1H), 7.99 (d, J=8.0 Hz, 1H), 7.93 (d, J=8.0 Hz, 1H), 7.86 (s, 1H), 7.65 (d, J=8.0 Hz, 1H), 7.17-7.10 (m, 2H), 6.97-6.91 (m, 2H), 5.43-5.31 (m, 1H), 5.20-5.02 (m, 1H), 3.77-3.65 (m, 4H), 2.81-2.62 (m, 7H), 2.53 (s, 3H), 2.44-2.19 (m, 3H), 2.14-1.96 (m, 2H), 1.89-1.79 (m, 2H), 1.78-1.64 (m, 3H), 1.55 (d, J=8.0 Hz, 3H), 1.37 (s, 9H), 1.30-1.18 (m, 2H); [M+H]⁺=866.4.

### Example 205: (R)-3-(tert-butyl)-N-(1-(4-(5-(5-(1-((1-(4-(2,4-dioxotetrahydropyrimidin-1(2H)-yl)phenyl)piperidin-4-yl)methyl)-4-fluoropiperidin-4-yl)pyridin-2-yl)-1H-pyrazolo[3,4-b]pyridin-3-yl)-2-methylphenyl)ethyl)-1,2,4-oxadiazole-5-carboxamide

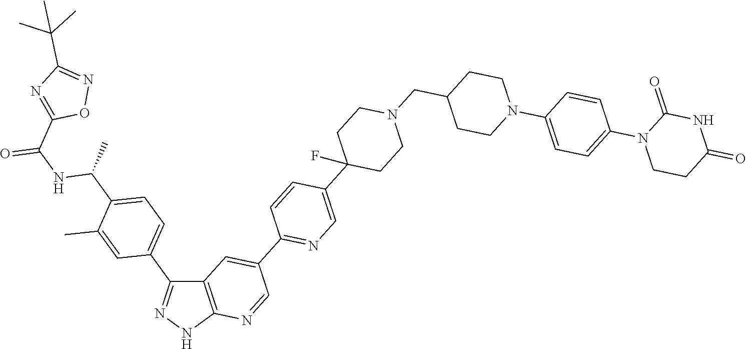 [SMILES: Cc1cc(-c2n[nH]c3ncc(-c4ccc(C5(F)CCN(CC6CCN(c7ccc(N8CCC(=O)NC8=O)cc7)CC6)CC5)cn4)cc23)ccc1[C@@H](C)NC(=O)c1nc(C(C)(C)C)no1]

### Step 1: tert-butyl 4-(6-bromopyridin-3-yl)-4-hydroxypiperidine-1-carboxylate

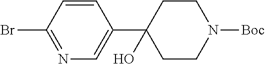 [SMILES: CC(C)(C)OC(=O)N1CCC(O)(c2ccc(Br)nc2)CC1]

To a solution of 2-bromo-5-iodopyridine (11.32 g, 40 mmol) in THF (110 mL) was added n-BuLi (2.5 M in hexane, 17.6 mL, 44 mmol) at −78° C. under a nitrogen atmosphere. The mixture was stirred at −78° C. for 1 h. Then, a solution of tert-butyl 4-oxopiperidine-1-carboxylate (8.9 g, 44.8 mmol) in THF (30 mL) was added dropwise to the mixture above. The resulting mixture was stirred at −78° C. for 1 h. The mixture was quenched by water (150 mL) and extracted with EtOAc (3×100 mL). The combined organic layers were dried over Na₂SO₄, filtered and concentrated under vacuum. The residue was purified by silica gel column chromatography, eluted with EtOAc/PE (2:3) to afford the product (7.1 g, 49.8%). [M+H]⁺=357.1.

### Step 2: tert-butyl 4-(6-bromopyridin-3-yl)-4-fluoropiperidine-1-carboxylate

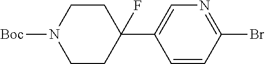 [SMILES: CC(C)(C)OC(=O)N1CCC(F)(c2ccc(Br)nc2)CC1]

To a solution of tert-butyl 4-(6-bromopyridin-3-yl)-4-hydroxypiperidine-1-carboxylate (2.7 g, 7.58 mmol) in THF (30 mL) was added DAST (1.9 g, 9.1 mmol) at −78° C. under a nitrogen atmosphere. The mixture was stirred at −50° C. for 1 h. The mixture was quenched by sat. NaHCO₃ (10 mL, aq) and extracted with DCM (3×100 mL). The combined organic layers were dried over Na₂SO₄, filtered and concentrated under vacuum. The residue was purified by silica gel column chromatography, eluted with EtOAc/PE (1:3) to afford the product (1.7 g, 62.7%). [M+H]⁺=359.1.

### Step 3: tert-butyl 4-(6-(3-(4-((R)-1-(3-(tert-butyl)-1,2,4-oxadiazole-5-carboxamido)ethyl)-3-methylphenyl)-1-(tetrahydro-2H-pyran-2-yl)-1H-pyrazolo[3,4-b]pyridin-5-yl)pyridin-3-yl)-4-fluoropiperidine-1-carboxylate

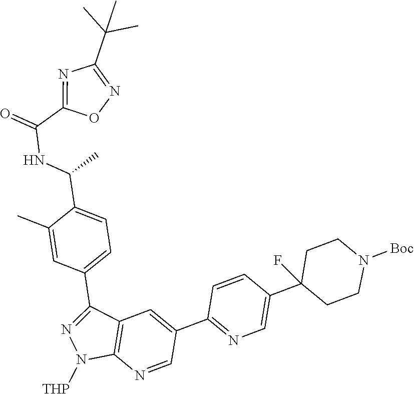

A mixture of 3-(tert-butyl)-N-((1R)-1-(2-methyl-4-(1-(tetrahydro-2H-pyran-2-yl)-5-(4,4,5,5-tetramethyl-1,3,2-dioxaborolan-2-yl)-1H-pyrazolo[3,4-b]pyridin-3-yl)phenyl)ethyl)-1,2,4-oxadiazole-5-carboxamide (236 mg, 0.38 mmol), tert-butyl 4-(6-bromopyridin-3-yl)-4-fluoropiperidine-1-carboxylate (165 mg, 0.46 mmol), Pd(dppf)Cl₂ (28.1 mg, 0.038 mmol) and K₂CO₃ (106 mg, 0.76 mmol) in dioxane (10 mL) and H₂O (2 mL) was stirred at 100° C. for 16 h under a nitrogen atmosphere. The mixture was concentrated under vacuum. The residue was purified by silica gel column chromatography, eluted with EtOAc/PE (2:1) to afford the product (200 mg, 67.8%). [M+H]⁺=767.4.

### Step 4: (R)-3-(tert-butyl)-N-(1-(4-(5-(5-(4-fluoropiperidin-4-yl)pyridin-2-yl)-1H-pyrazolo[3,4-b]pyridin-3-yl)-2-methylphenyl)ethyl)-1,2,4-oxadiazole-5-carboxamide hydrochloride

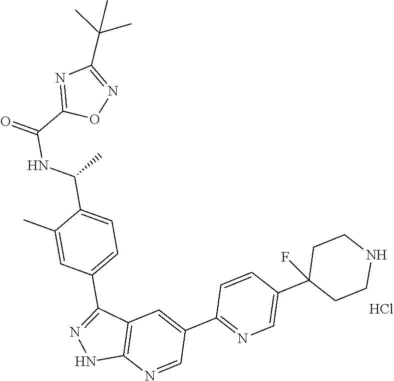 [SMILES: Cc1cc(-c2n[nH]c3ncc(-c4ccc(C5(F)CCNCC5)cn4)cc23)ccc1[C@@H](C)NC(=O)c1nc(C(C)(C)C)no1 ; Cl]

A mixture of tert-butyl 4-(6-(3-(4-((R)-1-(3-(tert-butyl)-1,2,4-oxadiazole-5-carboxamido)ethyl)-3-methylphenyl)-1-(tetrahydro-2H-pyran-2-yl)-1H-pyrazolo[3,4-b]pyridin-5-yl)pyridin-3-yl)-4-fluoropiperidine-1-carboxylate (200 mg) in 4 N HCl in dioxane (20 mL) was stirred at rt for 2 h. The mixture was concentrated under vacuum to afford the product (20 mg, crude), which was used in the next step directly. [M+H]⁺=583.3.

### Step 5: (R)-3-(tert-butyl)-N-(1-(4-(5-(5-(1-((1-(4-(2,4-dioxotetrahydropyrimidin-1(2H)-yl)phenyl)piperidin-4-yl)methyl)-4-fluoropiperidin-4-yl)pyridin-2-yl)-1H-pyrazolo[3,4-b]pyridin-3-yl)-2-methylphenyl)ethyl)-1,2,4-oxadiazole-5-carboxamide

A mixture of (R)-3-(tert-butyl)-N-(1-(4-(5-(5-(4-fluoropiperidin-4-yl)pyridin-2-yl)-1H-pyrazolo[3,4-b]pyridin-3-yl)-2-methylphenyl)ethyl)-1,2,4-oxadiazole-5-carboxamide hydrochloride (200 mg, 0.34 mmol), 1-(4-(2,4-dioxotetrahydropyrimidin-1(2H)-yl)phenyl)piperidine-4-carbaldehyde (124 mg, 0.41 mmol) and NaOAc (56 mg, 0.68 mmol) in MeOH (10 mL) and DCM (10 mL) was stirred at room temperature for 16 h. Then, STAB (145 mg, 0.68 mmol) was added to the mixture above and the mixture was stirred at room temperature for 6 h. The mixture was concentrated under vacuum. The residue was purified by silica gel column chromatography, eluted with MeOH/DCM (1:9) to afford the product (80.9 mg, 27.1%). ¹H NMR (400 MHz, DMSO) δ_{H} 13.94 (s, 1H), 10.26 (s, 1H), 9.92 (d, J=8.0 Hz, 1H), 9.31 (s, 1H), 9.07 (s, 1H), 8.81 (s, 1H), 8.27-8.20 (m, 1H), 7.99 (d, J=8.0 Hz, 1H), 7.92 (d, J=8.0 Hz, 1H), 7.86 (s, 1H), 7.65 (d, J=8.0 Hz, 1H), 7.18-7.09 (m, 2H), 6.99-6.90 (m, 2H), 5.42-5.31 (m, 1H), 3.78-3.64 (m, 4H), 2.91-2.79 (m, 2H), 2.74-2.63 (m, 4H), 2.53 (s, 3H), 2.39-2.08 (m, 6H), 2.05-1.94 (m, 2H), 1.88-1.80 (m, 2H), 1.78-1.60 (m, 1H), 1.55 (d, J=8.0 Hz, 3H), 1.37 (s, 9H), 1.31-1.19 (m, 2H). [M+H]⁺=868.4.

### Example 206: 3-(tert-butyl)-N—((R)-1-(4-(5-(4-(4-(2-((S)-2′,5′-dioxo-1,3-dihydrospiro[indene-2,3′-pyrrolidin]-5-yl)ethyl)piperazin-1-yl)phenyl)-1H-pyrazolo[3,4-b]pyridin-3-yl)-2-methylphenyl)ethyl)-1,2,4-oxadiazole-5-carboxamide

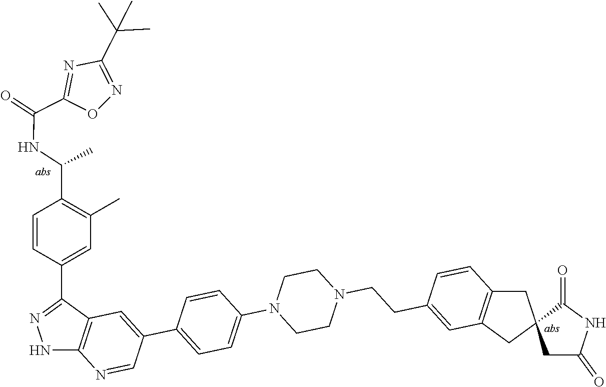 [SMILES: Cc1cc(-c2n[nH]c3ncc(-c4ccc(N5CCN(CCc6ccc7c(c6)C[C@]6(CC(=O)NC6=O)C7)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.79 (s, 1H), 11.26 (s, 1H), 9.91 (d, J=8.0 Hz, 1H), 8.82 (s, 1H), 8.58 (s, 1H), 7.92 (d, J=8.0 Hz, 1H), 7.85 (s, 1H), 7.69 (d, J=8.0 Hz, 2H), 7.62 (d, J=8.0 Hz, 1H), 7.17-6.99 (m, 5H), 5.42-5.28 (m, 1H), 3.22 (s, 6H), 3.01 (d, J=16.0, 4.0 Hz, 3H), 2.75 (t, J=8.0 Hz, 2H), 2.71 (s, 2H), 2.62 (s, 4H), 2.56 (t, J=8.0 Hz, 2H), 1.79 (s, 1H), 1.54 (d, J=8.0 Hz, 3H), 1.36 (s, 9H); [M+H]⁺=792.8.

### Example 207: 3-(tert-butyl)-N—((R)-1-(4-(5-(4-(4-(2-((R)-2′,5′-dioxo-1,3-dihydrospiro[indene-2,3′-pyrrolidin]-5-yl)ethyl)piperazin-1-yl)phenyl)-1H-pyrazolo[3,4-b]pyridin-3-yl)-2-methylphenyl)ethyl)-1,2,4-oxadiazole-5-carboxamide

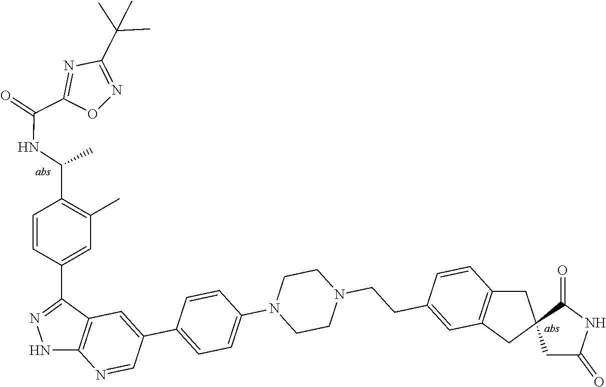 [SMILES: Cc1cc(-c2n[nH]c3ncc(-c4ccc(N5CCN(CCc6ccc7c(c6)C[C@@]6(CC(=O)NC6=O)C7)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.79 (s, 1H), 11.26 (s, 1H), 9.91 (d, J=8.0 Hz, 1H), 8.82 (s, 1H), 8.58 (s, 1H), 7.92 (d, J=8.0 Hz, 1H), 7.85 (s, 1H), 7.69 (d, J=8.0 Hz, 2H), 7.62 (d, J=8.0 Hz, 1H), 7.17-6.99 (m, 5H), 5.42-5.28 (m, 1H), 3.22 (s, 6H), 3.01 (d, J=16.0, 4.0 Hz, 3H), 2.75 (t, J=8.0 Hz, 2H), 2.71 (s, 2H), 2.62 (s, 4H), 2.56 (t, J=8.0 Hz, 2H), 1.79 (s, 1H), 1.54 (d, J=8.0 Hz, 3H), 1.36 (s, 9H); [M+H]⁺=792.7.

### Example 208 and 209:_5-(tert-butyl)-N—((R)-1-(4-(5-(4-(4-((1S,3s)-3-(4-((R)-2,6-dioxopiperidin-3-yl)phenyl)cyclobutyl)piperazin-1-yl)phenyl)-1H-pyrazolo[3,4-b]pyridin-3-yl)-2-methylphenyl)ethyl)-1,2,4-oxadiazole-3-carboxamide and 5-(tert-butyl)-N—((R)-1-(4-(5-(4-(4-((1R,3s)-3-(4-((S)-2,6-dioxopiperidin-3-yl)phenyl)cyclobutyl)piperazin-1-yl)phenyl)-1H-pyrazolo[3,4-b]pyridin-3-yl)-2-methylphenyl)ethyl)-1,2,4-oxadiazole-3-carboxamide

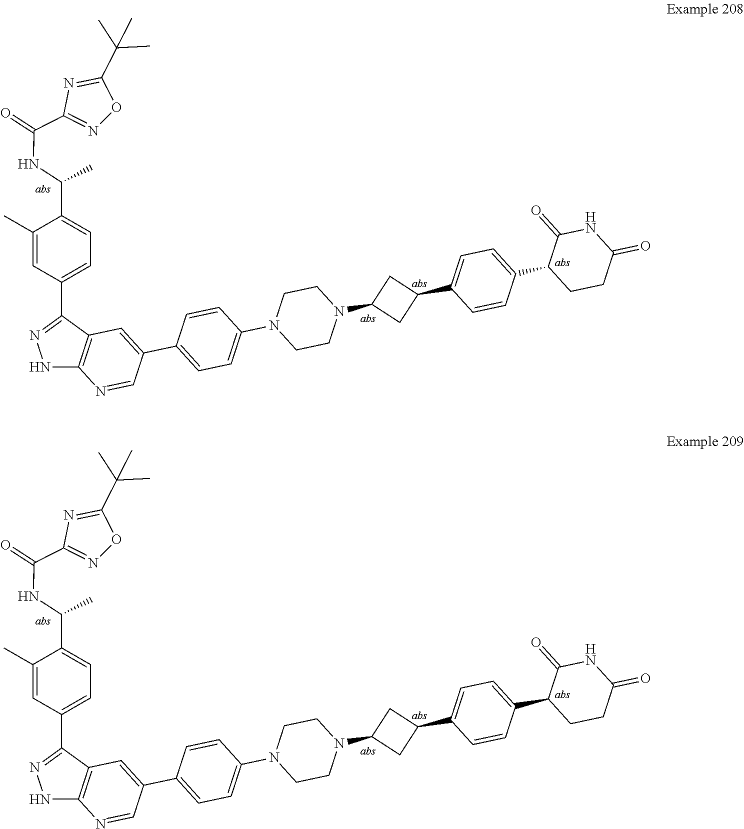 [SMILES: Cc1cc(-c2n[nH]c3ncc(-c4ccc(N5CCN([C@H]6C[C@@H](c7ccc([C@H]8CCC(=O)NC8=O)cc7)C6)CC5)cc4)cc23)ccc1[C@@H](C)NC(=O)c1noc(C(C)(C)C)n1 ; Cc1cc(-c2n[nH]c3ncc(-c4ccc(N5CCN([C@H]6C[C@@H](c7ccc([C@@H]8CCC(=O)NC8=O)cc7)C6)CC5)cc4)cc23)ccc1[C@@H](C)NC(=O)c1noc(C(C)(C)C)n1]

Each enantiomer was separated from Example 176 by using preparative HPLC on a CHIRALPAK IG-3 with Hex (0.2% IPAmine):(EtOH:DCM=1:1)=20:80 as an eluent. The enantiomeric excesses were determined by using HPLC on a CHIRALPAK IG-3 with Hex (0.2% IPAmine):(EtOH:DCM=1:1)=20:80 as an eluent at a flow rate of 1.0 mL/min. The first enantiomer Example 208 was eluted at the retention time of 1.287 min, and the other enantiomer Example 209 was eluted at the retention time of 2.058 min. Example 208: ¹H NMR (400 MHz, CDCl₃) δ_{H} 12.00 (s, 1H), 8.88 (s, 1H), 8.63 (s, 1H), 8.46 (s, 1H), 7.90-7.86 (m, 2H), 7.59-7.55 (m, 3H), 7.35-7.33 (m, 2H), 7.28-7.16 (m, 3H), 7.08 (d, J=8.4 Hz, 2H), 5.64-5.60 (m, 1H), 3.81-3.77 (m, 1H), 3.35-3.19 (m, 5H), 2.86-2.56 (m, 11H), 2.29 (brs, 2H), 2.11 (brs, 1H), 1.78-1.69 (m, 5H), 1.48 (s, 9H); [M+H]⁺=806.75. Example 209: ¹H NMR (400 MHz, CDCl₃) δ_{H} 11.65 (s, 1H), 8.86 (s, 1H), 8.46-8.44 (m, 2H), 7.89-7.86 (m, 2H), 7.59-7.56 (m, 3H), 7.32-7.28 (m, 2H), 7.20-7.16 (m, 3H), 7.08 (d, J=8.8 Hz, 2H), 5.64-5.60 (m, 1H), 3.81-3.77 (m, 1H), 3.35-3.19 (m, 5H), 2.86-2.56 (m, 11H), 2.28 (brs, 2H), 2.11 (brs, 1H), 1.70-1.64 (m, 5H), 1.48 (s, 9H); [M+H]⁺=806.65.

### Example 210: (R)-5-(tert-butyl)-N-(1-(4-(5-(4-(1-((1-(4-(2,4-dioxotetrahydropyrimidin-1(2H)-yl)phenyl)piperidin-4-yl)methyl)-4-methylpiperidin-4-yl)phenyl)-1H-pyrazolo[3,4-b]pyridin-3-yl)-2-methylphenyl)ethyl)-1,2,4-oxadiazole-3-carboxamide

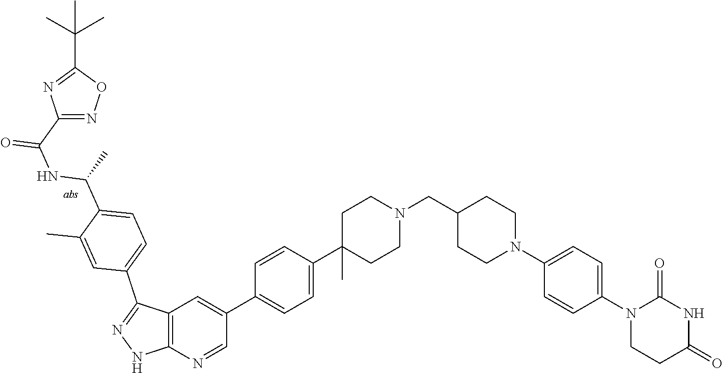

The titled compound was synthesized in the procedures similar to Example 1. ¹H NMR (400 MHz, DMSO) δ_{H} 13.86 (s, 1H), 10.26 (s, 1H), 9.92 (d, J=8.0 Hz, 1H), 8.88 (s, 1H), 8.69 (s, 1H), 7.94 (d, J=8.8 Hz, 1H), 7.87 (s, 1H), 7.81 (d, J=7.6 Hz, 2H), 7.62 (d, J=8.0 Hz, 1H), 7.53 (d, J=7.6 Hz, 2H), 7.13 (d, J=8.8 Hz, 2H), 6.93 (d, J=8.8 Hz, 2H), 5.41-5.30 (m, 1H), 3.68 (t, J=6.4 Hz, 4H), 3.11-2.69 (m, 9H), 2.50 (s, 3H), 2.33 (brs, 3H), 2.02-1.68 (m, 5H), 1.54 (d, J=6.8 Hz, 3H), 1.37 (s, 9H), 1.27 (brs, 5H); [M+H]⁺=863.9.

### Example 211: 5-(tert-butyl)-N-((1R)-1-(4-(5-(4-(1-(3-(4-(2,6-dioxopiperidin-3-yl)phenyl)cyclobutyl)-4-methylpiperidin-4-yl)phenyl)-1H-pyrazolo[3,4-b]pyridin-3-yl)-2-methylphenyl)ethyl)-1,2,4-oxadiazole-3-carboxamide

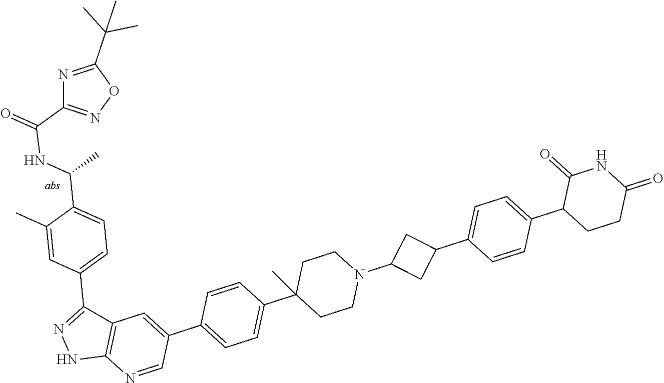 [SMILES: Cc1cc(-c2n[nH]c3ncc(-c4ccc(C5(C)CCN(C6CC(c7ccc(C8CCC(=O)NC8=O)cc7)C6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.86 (s, 1H), 10.82 (s, 1H), 9.92 (d, J=8.0 Hz, 1H), 8.87 (s, 1H), 8.69 (s, 1H), 7.94 (d, J=9.2 Hz, 1H), 7.87 (s, 1H), 7.81 (s, 2H), 7.62 (d, J=8.0 Hz, 1H), 7.55 (d, J=8.0 Hz, 2H), 7.31 (s, 2H), 7.16 (d, J=7.2 Hz, 2H), 5.35 (s, 1H), 3.82 (d, J=7.1 Hz, 1H), 3.20-2.55 (m, 9H), 2.50 (s, 3H), 2.40-2.08 (m, 5H), 1.99 (brs, 4H), 1.54 (d, J=6.8 Hz, 3H), 1.36 (s, 9H), 1.28 (brs, 3H); [M+H]⁺=819.8.

### Example 212: (R)-3-(tert-butyl)-N-(1-(4-(5-(4-(6-((1-(4-(2,4-dioxotetrahydropyrimidin-1(2H)-yl)phenyl)piperidin-4-yl)methyl)-2,6-diazaspiro[3.3]heptan-2-yl)phenyl)-1H-pyrazolo[3,4-b]pyridin-3-yl)-2-methylphenyl)ethyl)-1,2,4-oxadiazole-5-carboxamide

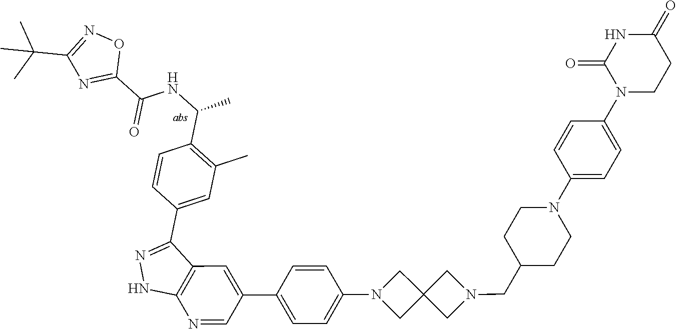 [SMILES: Cc1cc(-c2n[nH]c3ncc(-c4ccc(N5CC6(CN(CC7CCN(c8ccc(N9CCC(=O)NC9=O)cc8)CC7)C6)C5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.76 (s, 1H), 10.26 (s, 2H), 9.90 (d, J=8.0 Hz, 2H), 8.79 (s, 1H), 8.54 (s, 1H), 7.91 (d, J=8.0 Hz, 1H), 7.84 (s, 1H), 7.69-7.58 (m, 3H), 7.13 (d, J=8.0 Hz, 2H), 6.93 (d, J=8.0 Hz, 2H), 6.57 (d, J=8.0 Hz, 2H), 5.35 (s, 2H), 3.96 (s, 4H), 3.69 (s, 5H), 3.32-3.28 (m, 5H), 2.74-2.56 (m, 6H), 1.91 (s, 1H), 1.75 (d, J=12.0 Hz, 2H), 1.53 (d, J=4.0 Hz, 3H), 1.37 (s, 9H), 1.30-1.19 (m, 3H); [M+H]⁺=863.0.

### Example 213: (R)—N-(1-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylphenyl)ethyl)-3-(1-methylcyclopropyl)-1,2,4-oxadiazole-5-carboxamide

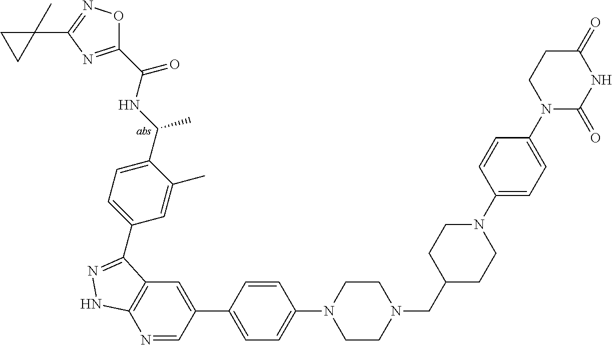 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1[C@@H](C)NC(=O)c1nc(C2(C)CC2)no1]

The titled compound was synthesized in the procedures similar to Example 1. ¹H NMR (400 MHz, DMSO) δ_{H} 13.79 (s, 1H), 10.26 (s, 1H), 9.84 (d, J=7.6 Hz, 1H), 8.82 (s, 1H), 8.58 (s, 1H), 7.92 (d, J=8.0 Hz, 1H), 7.85 (s, 1H), 7.75-7.65 (m, 2H), 7.60 (d, J=8.0 Hz, 1H), 7.18-7.02 (m, 4H), 6.94 (d, J=8.4 Hz, 2H), 5.38-5.29 (m, 1H), 3.76-3.64 (m, 4H), 3.26-3.15 (m, 4H), 2.74-2.62 (m, 4H), 2.60-2.52 (m, 2H), 2.30-2.18 (m, 1H), 1.88-1.64 (m, 3H), 1.52 (d, J=6.8 Hz, 3H), 1.48 (s, 3H), 1.32-1.66 (m, 4H), 1.01-0.95 (s, 2H); [M+H]⁺=848.8.

### Example 214: 5-(tert-butyl)-N-((1R)-1-(4-(5-(4-(4-((1s,3s)-3-(4-(2,6-dioxopiperidin-3-yl)phenyl)cyclobutyl)piperazin-1-yl)phenyl)-1H-pyrazolo[3,4-b]pyridin-3-yl)-2-methylphenyl)ethyl)-1,2,4-oxadiazole-3-carboxamide

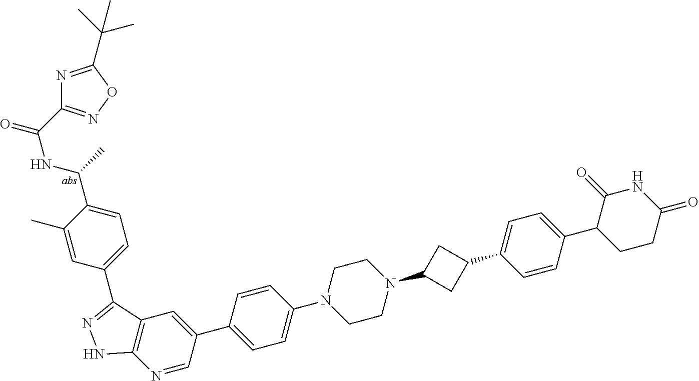

The titled compound was synthesized in the procedures similar to Example 1. ¹H NMR (400 MHz, DMSO) δ_{H} 13.76 (s, 1H), 10.82 (s, 1H), 9.46 (d, J=8.0 Hz, 1H), 8.82 (s, 1H), 8.58 (s, 1H), 7.91 (d, J=8.2 Hz, 1H), 7.85 (s, 1H), 7.69 (d, J=8.5 Hz, 2H), 7.60 (d, J=8.1 Hz, 1H), 7.28 (d, J=8.0 Hz, 2H), 7.17 (d, J=7.8 Hz, 2H), 7.08 (d, J=8.3 Hz, 2H), 5.36 (s, 1H), 3.83 (d, J=6.3 Hz, 1H), 3.47 (s, 1H), 3.24 (s, 4H), 2.86-3.05 (m, 2H), 2.59-2.74 (m, 2H), 2.31-2.54 (m, 5H), 2.11-2.25 (m, 3H), 1.98-2.08 (m, 1H), 1.51 (d, J=6.9 Hz, 3H), 1.42 (s, 9H); [M+H]⁺=807.0.

### Example 215: 3-(tert-butyl)-N-((1R)-1-(4-(5-(4-(4-(4-(2,6-dioxopiperidin-3-yl)-3-fluorobenzyl)piperazin-1-yl)phenyl)-1H-pyrazolo[3,4-b]pyridin-3-yl)-2-methylphenyl)ethyl)-1,2,4-oxadiazole-5-carboxamide

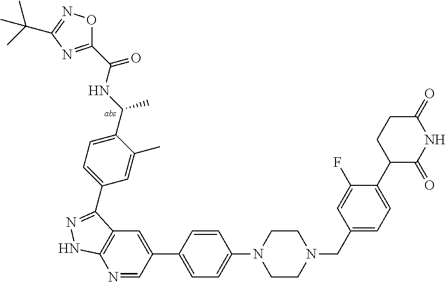 [SMILES: Cc1cc(-c2n[nH]c3ncc(-c4ccc(N5CCN(Cc6ccc(C7CCC(=O)NC7=O)c(F)c6)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.78 (s, 1H), 10.89 (s, 1H), 9.90 (d, J=7.6 Hz, 1H), 8.82 (s, 1H), 8.58 (s, 1H), 7.92 (d, J=8.4 Hz, 1H), 7.85 (s, 1H), 7.69 (d, J=8.4 Hz, 2H), 7.62 (d, J=8.4 Hz, 1H), 7.30 (s, 1H), 7.18 (s, 2H), 7.06 (d, J=8.4 Hz, 2H), 5.40-5.30 (m, 1H), 4.10-4.00 (m, 1H), 3.65-3.50 (m, 2H), 3.28-3.18 (m, 3H), 2.80-2.69 (m, 1H), 2.65-2.52 (m, 3H), 2.28-2.16 (m, 1H), 2.08-1.96 (m, 1H), 1.53 (d, J=6.8 Hz, 3H), 1.36 (s, 9H); [M+H]⁺=785.6.

### Example 216: 3-(tert-butyl)-N-((1R)-1-(4-(5-(4-(4-(3-(4-(2,6-dioxopiperidin-3-yl)-3-fluorophenyl)cyclopentyl)piperazin-1-yl)phenyl)-1H-pyrazolo[3,4-b]pyridin-3-yl)-2-methylphenyl)ethyl)-1,2,4-oxadiazole-5-carboxamide

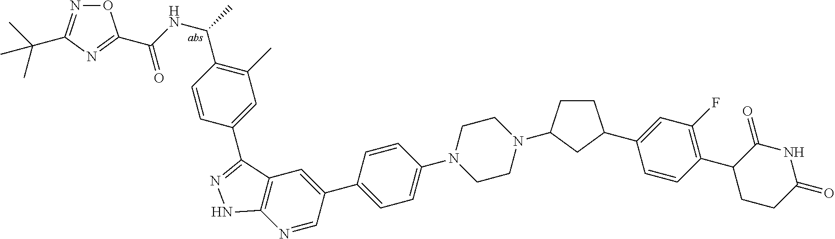 [SMILES: Cc1cc(-c2n[nH]c3ncc(-c4ccc(N5CCN(C6CCC(c7ccc(C8CCC(=O)NC8=O)c(F)c7)C6)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.78 (s, 1H), 10.85 (s, 1H), 9.90 (d, J=8.0 Hz, 1H), 8.82 (s, 1H), 8.59 (s, 1H), 7.92 (d, J=8.0 Hz, 1H), 7.85 (s, 1H), 7.70 (d, J=8.0 Hz, 2H), 7.62 (d, J=8.0 Hz, 1H), 7.41-7.29 (m, 1H), 7.12-7.00 (m, 4H), 5.53-5.14 (m, 1H), 3.87 (dd, J=8.0, 2.0 Hz, 1H), 3.30-3.16 (m, 7H), 2.74-2.58 (m, 5H), 2.52 (s, 2H), 2.23 (dd, J=12.0, 4.0 Hz, 2H), 2.03 (s, 4H), 1.70 (s, 3H), 1.54 (d, J=4.0 Hz, 3H), 1.36 (s, 9H); [M+H]⁺=838.8.

### Example 217: (R)-3-(tert-butyl)-N-(1-(4-(5-(4-(4-(4-(2,4-dioxoimidazolidin-1-yl)phenethyl)piperazin-1-yl)phenyl)-1H-pyrazolo[3,4-b]pyridin-3-yl)-2-methylphenyl)ethyl)-1,2,4-oxadiazole-5-carboxamide

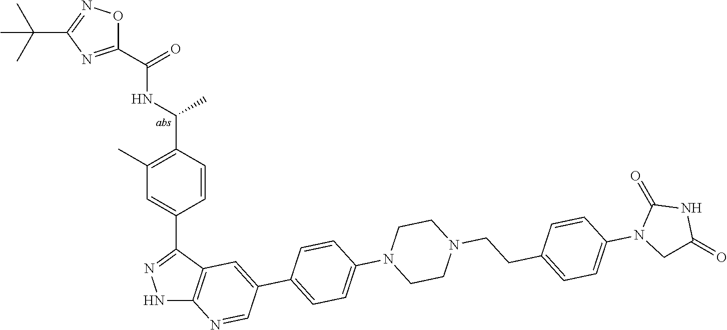 [SMILES: Cc1cc(-c2n[nH]c3ncc(-c4ccc(N5CCN(CCc6ccc(N7CC(=O)NC7=O)cc6)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.79 (s, 1H), 11.17 (s, 1H), 9.90 (d, J=8.0 Hz, 1H), 8.83 (s, 1H), 8.59 (s, 1H), 7.92 (d, J=8.0 Hz, 1H), 7.85 (s, 1H), 7.73 (d, J=8.0 Hz, 2H), 7.62 (d, J=8.0 Hz, 1H), 7.54 (d, J=8.0 Hz, 2H), 7.28 (d, J=8.0 Hz, 2H), 7.12 (d, J=8.0 Hz, 2H), 5.40-5.30 (m, 1H), 4.44 (s, 3H), 3.09 (s, 4H), 2.91 (s, 6H), 1.54 (d, J=6.8 Hz, 3H), 1.37 (s, 9H), 1.23 (s, 4H), 1.18 (t, J=7.3 Hz, 3H); [M+H]⁺=767.9.

### Example 218: (R)-3-(tert-butyl)-N-(1-(4-(5-(4-(4-((1-(4-(2,4-dioxoimidazolidin-1-yl)phenyl)piperidin-4-yl)methyl)piperazin-1-yl)phenyl)-1H-pyrazolo[3,4-b]pyridin-3-yl)-2-methylphenyl)ethyl)-1,2,4-oxadiazole-5-carboxamide

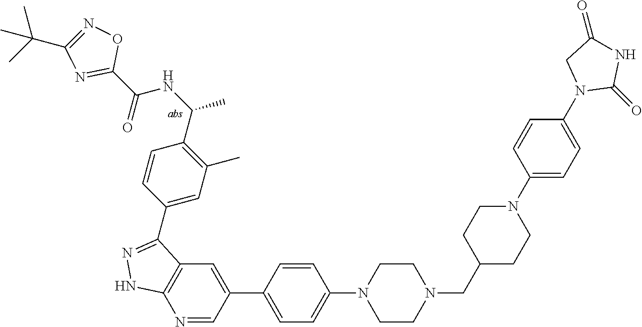 [SMILES: Cc1cc(-c2n[nH]c3ncc(-c4ccc(N5CCN(CC6CCN(c7ccc(N8CC(=O)NC8=O)cc7)CC6)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.77 (s, 1H), 11.04 (s, 1H), 9.90 (d, J=8.0 Hz, 1H), 8.82 (s, 1H), 8.58 (s, 1H), 7.92 (d, J=8.0 Hz, 1H), 7.85 (s, 1H), 7.69 (d, J=8.0 Hz, 2H), 7.62 (d, J=8.0 Hz, 1H), 7.40 (d, J=8.0 Hz, 2H), 7.07 (d, J=8.0 Hz, 2H), 6.94 (d, J=9.0 Hz, 2H), 5.44-5.22 (m, 1H), 4.38 (s, 2H), 3.64 (d, J=12.0 Hz, 3H), 3.22 (s, 4H), 2.70-2.59 (m, 3H), 2.53 (s, 4H), 2.24 (s, 2H), 1.82 (d, J=12.0 Hz, 2H), 1.71 (s, 2H), 1.54 (d, J=4.0 Hz, 3H), 1.36 (s, 9H), 1.24 (s, 2H); [M+H]⁺=836.8.

### Example 219: 3-(tert-butyl)-N-(4-(5-(4-(1-((1-(4-(2,4-dioxotetrahydropyrimidin-1(2H)-yl)phenyl)piperidin-4-yl)methyl)piperidin-4-yl)phenyl)-1H-pyrazolo[3,4-b]pyridin-3-yl)-3-fluoro-2-methylbenzyl)-1,2,4-oxadiazole-5-carboxamide

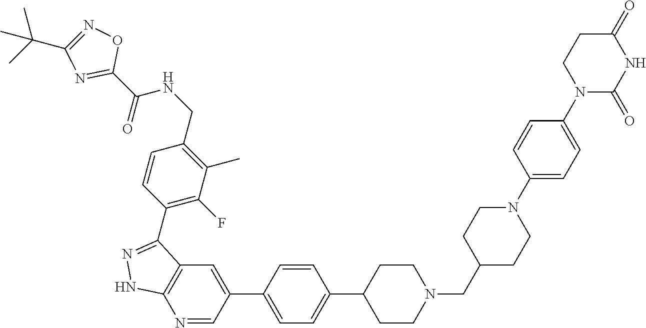 [SMILES: Cc1c(CNC(=O)c2nc(C(C)(C)C)no2)ccc(-c2n[nH]c3ncc(-c4ccc(C5CCN(CC6CCN(c7ccc(N8CCC(=O)NC8=O)cc7)CC6)CC5)cc4)cc23)c1F]

The titled compound was synthesized in the procedures similar to Example 1. ¹H NMR (400 MHz, DMSO) δ_{H} 14.05 (s, 1H), 10.27 (s, 1H), 9.91 (t, J=4.0 Hz, 1H), 8.89 (d, J=2.0 Hz, 1H), 8.39 (s, 1H), 7.78-7.60 (m, 3H), 7.39 (d, J=8.0 Hz, 2H), 7.31 (d, J=8.0 Hz, 1H), 7.16 (d, J=8.0 Hz, 2H), 6.96 (d, J=8.0 Hz, 2H), 4.57 (d, J=4.0 Hz, 2H), 3.80-3.56 (m, 6H), 3.06 (s, 3H), 2.89 (s, 2H), 2.77-2.63 (m, 4H), 2.36 (s, 3H), 2.02 (s, 4H), 1.93-1.83 (m, 3H), 1.41-1.31 (m, 11H); [M+H]⁺=853.9.

### Example 220: 3-(tert-butyl)-N-(4-(5-(4-(1-((1-(4-(2,4-dioxotetrahydropyrimidin-1(2H)-yl)phenyl)piperidin-4-yl)methyl)piperidin-4-yl)phenyl)-1H-pyrazolo[3,4-b]pyridin-3-yl)-5-fluoro-2-methylbenzyl)-1,2,4-oxadiazole-5-carboxamide

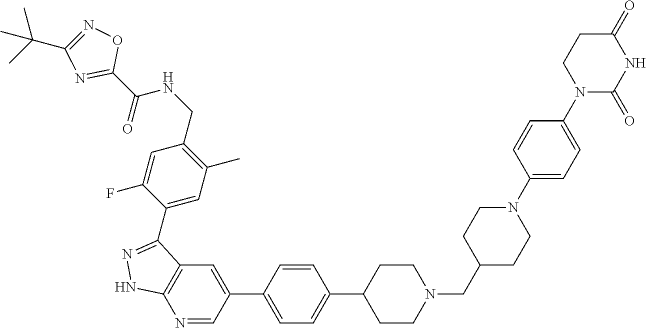 [SMILES: Cc1cc(-c2n[nH]c3ncc(-c4ccc(C5CCN(CC6CCN(c7ccc(N8CCC(=O)NC8=O)cc7)CC6)CC5)cc4)cc23)c(F)cc1CNC(=O)c1nc(C(C)(C)C)no1]

The titled compound was synthesized in the procedures similar to Example 1. ¹H NMR (400 MHz, DMSO) δ_{H} 14.06 (s, 1H), 10.27 (s, 1H), 9.89 (t, J=4.0 Hz, 1H), 8.89 (d, J=2.0 Hz, 1H), 8.37 (s, 1H), 7.78-7.65 (m, 3H), 7.79-7.66 (m, 3H), 7.39 (d, J=8.0 Hz, 2H), 7.31 (d, J=12.0 Hz, 1H), 7.15 (d, J=8.0 Hz, 2H), 6.96 (d, J=8.0 Hz, 2H), 4.52 (d, J=4.0 Hz, 2H), 3.77-3.51 (m, 6H), 3.06 (s, 4H), 2.89 (s, 1H), 2.77-2.63 (m, 4H), 2.41 (s, 3H), 2.16-1.95 (m, 5H), 1.89 (d, J=12.0 Hz, 2H), 1.38 (s, 9H), 1.36-1.27 (m, 2H); [M+H]⁺=853.8.

### Example 221: 5-(tert-butyl)-N-(4-(5-(4-(1-((1-(4-(2,4-dioxotetrahydropyrimidin-1(2H)-yl)phenyl)piperidin-4-yl)methyl)piperidin-4-yl)phenyl)-1H-pyrazolo[3,4-b]pyridin-3-yl)-3-fluoro-2-methylbenzyl)-1,2,4-oxadiazole-3-carboxamide

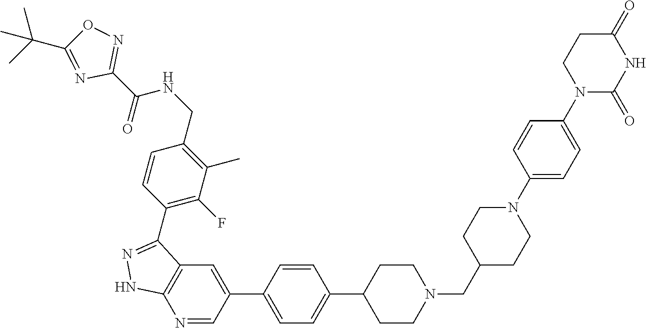 [SMILES: Cc1c(CNC(=O)c2noc(C(C)(C)C)n2)ccc(-c2n[nH]c3ncc(-c4ccc(C5CCN(CC6CCN(c7ccc(N8CCC(=O)NC8=O)cc7)CC6)CC5)cc4)cc23)c1F]

The titled compound was synthesized in the procedures similar to Example 1. ¹H NMR (400 MHz, DMSO) δ_{H} 14.04 (s, 1H), 10.26 (s, 1H), 9.53 (t, J=4.0 Hz, 1H), 8.88 (s, 1H), 8.39 (s, 1H), 7.79-7.62 (m, 3H), 7.39 (d, J=8.1 Hz, 2H), 7.27 (d, J=8.0 Hz, 1H), 7.15 (d, J=8.0 Hz, 2H), 6.96 (d, J=8.0 Hz, 2H), 4.56 (d, J=4.0 Hz, 2H), 3.77-3.57 (m, 5H), 3.30-3.24 (m, 2H), 3.12-2.80 (m, 5H), 2.77-2.66 (m, 4H), 2.36 (s, 3H), 2.22-1.98 (m, 4H), 1.92-1.80 (m, 2H), 1.44 (s, 9H), 1.39-1.28 (m, 2H); [M+H]⁺=854.0.

### Example 222: 5-(tert-butyl)-N-(4-(5-(4-(1-((1-(4-(2,4-dioxotetrahydropyrimidin-1(2H)-yl)phenyl)piperidin-4-yl)methyl)piperidin-4-yl)phenyl)-1H-pyrazolo[3,4-b]pyridin-3-yl)-5-fluoro-2-methylbenzyl)-1,2,4-oxadiazole-3-carboxamide

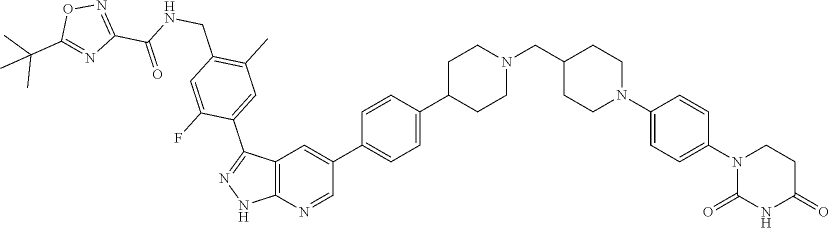 [SMILES: Cc1cc(-c2n[nH]c3ncc(-c4ccc(C5CCN(CC6CCN(c7ccc(N8CCC(=O)NC8=O)cc7)CC6)CC5)cc4)cc23)c(F)cc1CNC(=O)c1noc(C(C)(C)C)n1]

The titled compound was synthesized in the procedures similar to Example 1. ¹H NMR (400 MHz, DMSO) δ_{H} 14.03 (s, 1H), 10.31 (s, 1H), 9.54 (t, J=4.0 Hz, 1H), 8.89 (d, J=2.0 Hz, 1H), 8.38 (s, 1H), 7.78-7.68 (m, 3H), 7.39 (d, J=8.0 Hz, 2H), 7.24 (d, J=12.0 Hz, 3H), 7.15 (s, 1H), 4.52 (d, J=4.0 Hz, 2H), 3.72 (t, J=8.0 Hz, 4H), 3.65 (d, J=12.0 Hz, 3H), 3.51 (s, 2H), 3.31 (s, 4H), 3.10-3.05 (m, 3H), 2.94-2.90 (m, 1H), 2.69 (t, J=4.0 Hz, 2H), 2.40 (s, 3H), 2.21-2.08 (m, 3H), 2.04-1.92 (m, 4H), 1.44 (s, 9H); [M+H]⁺=853.9.

### Example 223 and 224: 5-(tert-butyl)-N—((R)-1-(4-(5-(4-(4-((1S,3r)-3-(4-((S)-2,6-dioxopiperidin-3-yl)phenyl)cyclobutyl)piperazin-1-yl)phenyl)-1H-pyrazolo[3,4-b]pyridin-3-yl)-2-methylphenyl)ethyl)-1,2,4-oxadiazole-3-carboxamide and 5-(tert-butyl)-N—((R)-1-(4-(5-(4-(4-((1S,3r)-3-(4-((R)-2,6-dioxopiperidin-3-yl)phenyl)cyclobutyl)piperazin-1-yl)phenyl)-1H-pyrazolo[3,4-b]pyridin-3-yl)-2-methylphenyl)ethyl)-1,2,4-oxadiazole-3-carboxamide

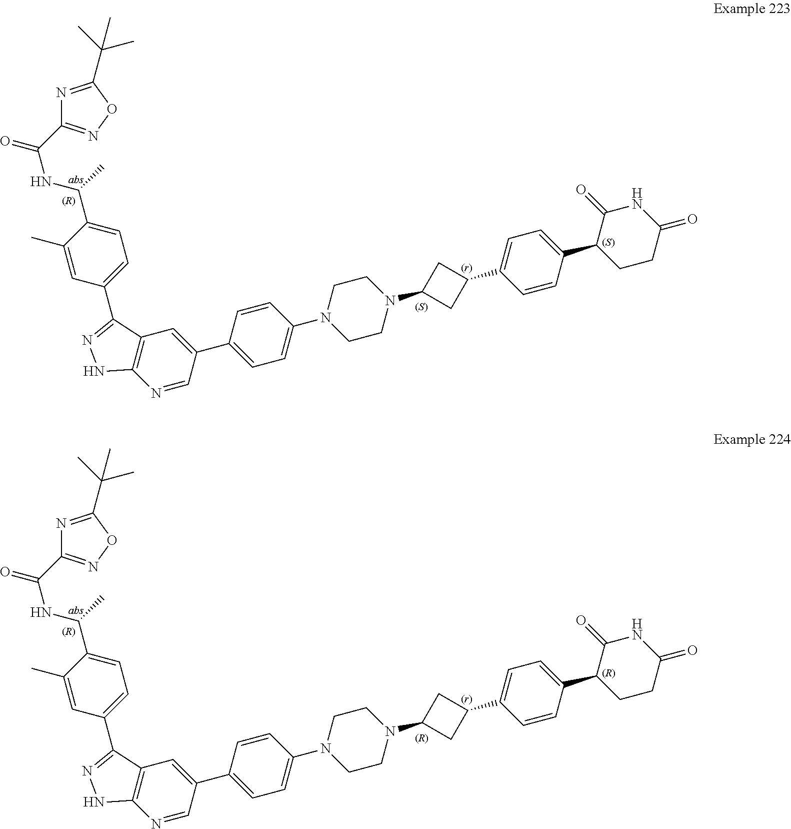

The Example 214 (53 mg) was separated by Prep-Chiral-HPLC with following conditions: Column: CHIRALPAK IG, 2*25 cm, 5 μm; Mobile Phase A: Hexane (0.2% DEA), Mobile Phase B: EtOH:DCM=1:1; Flow rate: 20 mL/min; Gradient: 90% B to 90% B in 9 min; Detector: 220/254 nm; RT1: 4.449 min; RT2: 7.026 min; Sample Solvent: EtOH:DCM=1:1; Injection Volume: 1.2 mL; Number Of Runs: 3; This resulted in Example 223 (RT1: 1.164 min) (13.0 mg, 24.53%) and Example 224 (RT2:2.190 min), (18.31 mg, 34.55%). Example 223: ¹H NMR (400 MHz, DMSO) δ_{H} 13.78 (s, 1H), 10.84 (s, 1H), 9.50 (d, J=8.0 Hz, 1H), 8.82 (s, 1H), 8.58 (s, 1H), 7.91 (d, J=8.2 Hz, 1H), 7.85 (s, 1H), 7.70 (d, J=8.5 Hz, 2H), 7.60 (d, J=8.1 Hz, 1H), 7.28 (d, J=8.0 Hz, 2H), 7.18 (d, J=7.8 Hz, 2H), 7.08 (d, J=8.3 Hz, 2H), 5.36 (s, 1H), 3.83 (d, J=6.3 Hz, 1H), 3.47 (s, 1H), 3.24 (s, 4H), 2.84-2.98 (m, 1H), 2.55-2.79 (m, 2H), 2.35-2.55 (m, 6H), 2.11-2.29 (m, 3H), 2.01-2.09 (m, 1H), 1.50 (d, J=6.9 Hz, 3H), 1.43 (s, 9H); [M+H]⁺=806.45. Example 224: ¹HNMR (400 MHz, DMSO) δ_{H} 13.78 (s, 1H), 10.84 (s, 1H), 9.50 (d, J=8.0 Hz, 1H), 8.82 (s, 1H), 8.58 (s, 1H), 7.91 (d, J=8.2 Hz, 1H), 7.86 (s, 1H), 7.69 (d, J=8.5 Hz, 2H), 7.60 (d, J=8.1 Hz, 1H), 7.30 (d, J=8.0 Hz, 2H), 7.18 (d, J=7.8 Hz, 2H), 7.08 (d, J=8.3 Hz, 2H), 5.37 (s, 1H), 3.83 (d, J=6.3 Hz, 1H), 3.47 (s, 1H), 3.24 (s, 4H), 2.84-2.98 (m, 1H), 2.58-2.75 (m, 2H), 2.35-2.55 (m, 6H), 2.11-2.29 (m, 3H), 2.01-2.09 (m, 1H), 1.51 (d, J=6.9 Hz, 3H), 1.43 (s, 9H), 1.24 (s, 1H); [M+H]⁺=806.70.

### Example 225: (R)-5-(tert-butyl)-N-(1-(4-(5-(4-(4-((1-(4-(2,4-dioxotetrahydropyrimidin-1(2H)-yl)phenyl)piperidin-4-yl)methyl)piperazin-1-yl)phenyl)-1H-pyrazolo[3,4-b]pyridin-3-yl)-2-methylphenyl)ethyl)-1,3,4-oxadiazole-2-carboxamide

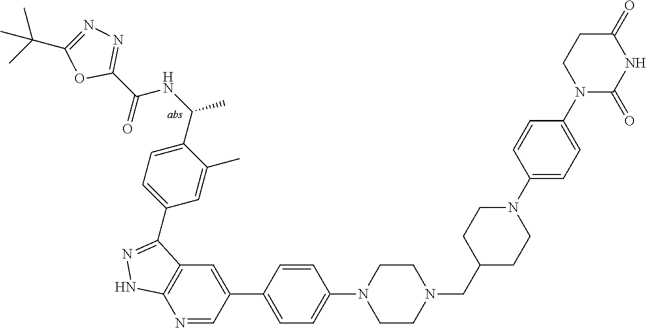 [SMILES: Cc1cc(-c2n[nH]c3ncc(-c4ccc(N5CCN(CC6CCN(c7ccc(N8CCC(=O)NC8=O)cc7)CC6)CC5)cc4)cc23)ccc1[C@@H](C)NC(=O)c1nnc(C(C)(C)C)o1]

The titled compound was synthesized in the procedures similar to Example 1. ¹H NMR (400 MHz, DMSO) δ_{H} 13.78 (s, 1H), 10.25 (s, 1H), 9.85 (d, J=6.4 Hz, 1H), 8.82 (s, 1H), 8.57 (s, 1H), 7.91 (d, J=6.8 Hz, 1H), 7.84 (s, 1H), 7.69 (d, J=6.0 Hz, 2H), 7.62 (d, J=6.4 Hz, 1H), 7.15-7.13 (m, 2H), 7.10-7.04 (m, 2H), 6.96-6.90 (m, 2H), 5.40-5.28 (m, 1H), 3.73-3.63 (m, 5H), 3.25-3.15 (m, 4H), 2.70-2.66 (m, 3H), 2.57-2.52 (m, 2H), 2.28-2.20 (m, 2H), 1.86-1.66 (m, 4H), 1.53 (d, J=5.6 Hz, 3H), 1.39 (s, 9H); [M+H]⁺=850.8.

### Example 226: 3-(tert-butyl)-N—((R)-1-(4-(5-(4-(4-(2-((2-((R)-2,6-dioxopiperidin-3-yl)-3-oxoisoindolin-4-yl)oxy)ethyl)piperazin-1-yl)phenyl)-1H-pyrazolo[3,4-b]pyridin-3-yl)-2-methylphenyl)ethyl)-1,2,4-oxadiazole-5-carboxamide

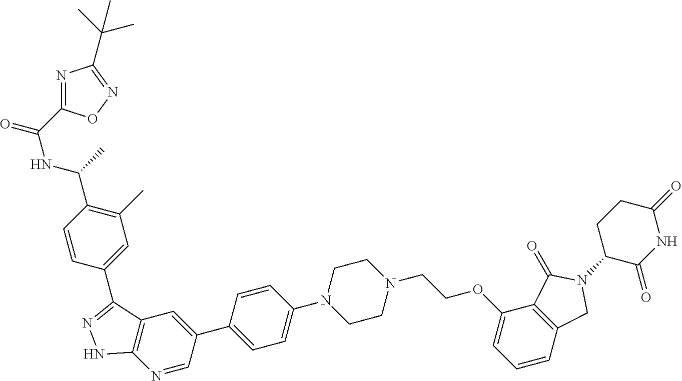 [SMILES: Cc1cc(-c2n[nH]c3ncc(-c4ccc(N5CCN(CCOc6cccc7c6C(=O)N([C@@H]6CCC(=O)NC6=O)C7)CC5)cc4)cc23)ccc1[C@@H](C)NC(=O)c1nc(C(C)(C)C)no1]

The titled compound was synthesized in the procedures similar to Example 1. ¹H NMR (400 MHz, DMSO) δ_{H} 13.77 (s, 1H), 10.98 (s, 1H), 9.90 (d, J=5.0 Hz, 1H), 8.81 (s, 1H), 8.57 (s, 1H), 7.92 (d, J=10.0 Hz, 1H), 7.85 (s, 1H), 7.68 (d, J=8.0 Hz, 1H), 7.61 (d, J=5.0 Hz, 1H), 7.50 (t, J=5.0 Hz, 1H), 7.32 (t, J=5.0 Hz, 2H), 7.06 (d, J=10.0 Hz, 2H), 5.39-5.31 (m, 1H), 5.15-5.08 (m, 1H), 4.42-4.36 (m, 1H), 4.33-4.22 (m, 3H), 3.25-3.18 (m, 4H), 2.96-2.87 (m, 1H), 2.85-2.78 (m, 2H), 2.71-2.65 (m, 4H), 2.62-2.55 (m, 1H), 2.51 (s, 3H), 2.47-2.42 (m, 1H), 2.04-1.96 (m, 1H), 1.53 (d, J=5.0 Hz, 3H), 1.36 (s, 9H), 1.25-1.22 (m, 1H); [M+H]⁺=851.4.

### Example 227: 5-(tert-butyl)-N-((1R)-1-(4-(5-(4-(1-(4-(2,6-dioxopiperidin-3-yl)phenethyl)piperidin-4-yl)phenyl)-1H-pyrazolo[3,4-b]pyridin-3-yl)-2-methylphenyl)ethyl)-1,2,4-oxadiazole-3-carboxamide

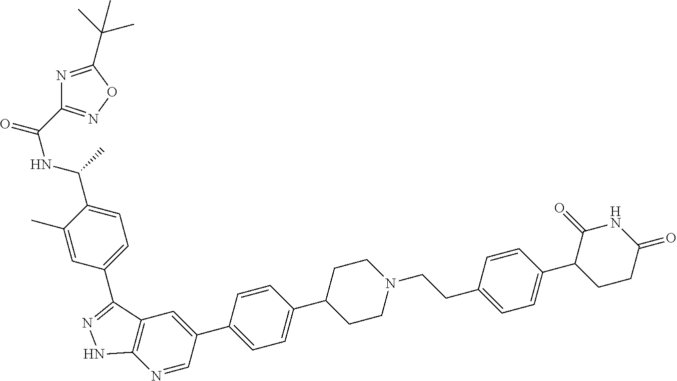 [SMILES: Cc1cc(-c2n[nH]c3ncc(-c4ccc(C5CCN(CCc6ccc(C7CCC(=O)NC7=O)cc6)CC5)cc4)cc23)ccc1[C@@H](C)NC(=O)c1noc(C(C)(C)C)n1]

The titled compound was synthesized in the procedures similar to Example 1. ¹H NMR (400 MHz, DMSO) δ_{H} 13.84 (s, 1H), 10.84 (s, 1H), 9.49 (s, 1H), 8.85 (s, 1H), 8.66 (s, 1H), 7.92-7.90 (m, 1H), 7.86 (s, 1H), 7.78-7.74 (m, 2H), 7.61 (d, J=2.1 Hz, 1H), 7.41 (d, J=6.8 Hz, 2H), 7.25-7.18 (m, 4H), 5.37-5.35 (m, 1H), 3.85-3.81 (m, 1H), 3.09-2.95 (m, 3H), 2.85-2.71 (m, 2H), 2.68-2.65 (m, 2H), 2.50 (s, 3H), 2.47-2.42 (m, 3H), 2.36-2.32 (m, 1H), 2.18-2.16 (m, 2H), 2.12-1.98 (m, 1H), 1.85-1.64 (m, 3H), 1.52 (d, J=4.0 Hz, 3H), 1.42 (s, 9H); [M+H]⁺=779.6.

### Example 228: 3-(tert-butyl)-N-(4-(5-(6-(4-((1-(4-(2,4-dioxotetrahydropyrimidin-1(2H)-yl)-3-methylphenyl)piperidin-4-yl)methyl)piperazin-1-yl)pyridin-3-yl)-1H-pyrazolo[3,4-b]pyridin-3-yl)-2-methylbenzyl)-1,2,4-oxadiazole-5-carboxamide

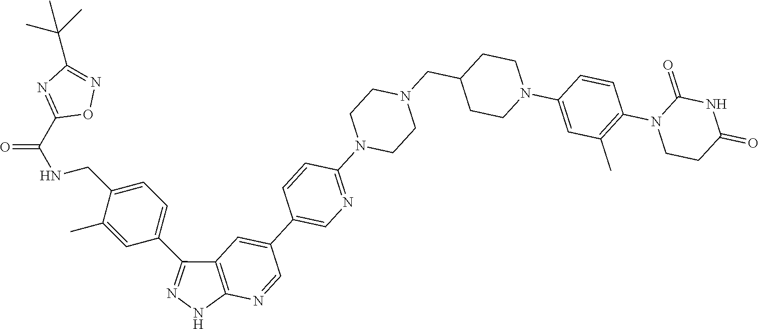 [SMILES: Cc1cc(-c2n[nH]c3ncc(-c4ccc(N5CCN(CC6CCN(c7ccc(N8CCC(=O)NC8=O)c(C)c7)CC6)CC5)nc4)cc23)ccc1CNC(=O)c1nc(C(C)(C)C)no1]

The titled compound was synthesized in the procedures similar to Example 1. ¹H NMR (400 MHz, DMSO) δ_{H} 13.82 (s, 1H), 10.26 (s, 1H), 9.87 (s, 1H), 8.83 (s, 1H), 8.65 (s, 1H), 8.60 (s, 1H), 8.03 (d, J=8.0 Hz, 1H), 7.94-7.91 (m, 2H), 7.42 (d, J=8.0 Hz, 1H), 7.06-6.95 (m, 2H), 6.82-6.77 (m, 2H), 4.53 (s, 2H), 3.71-3.69 (m, 3H), 3.57-3.48 (m, 5m), 2.70-2.68 (m, 5H), 2.54-2.33 (m, 5H), 2.28-2.24 (m, 2H), 2.12 (s, 3H), 1.84-1.74 (m, 4H), 1.37 (s, 9H), 1.23-1.21 (m, 2H); [M+H]⁺=851.8.

### Example 229: 5-(tert-butyl)-N-(4-(5-(4-(4-(2-(5-(2,6-dioxopiperidin-3-yl)pyridin-2-yl)ethyl)piperazin-1-yl)phenyl)-1H-pyrazolo[3,4-b]pyridin-3-yl)-2-methylbenzyl)-1,2,4-oxadiazole-3-carboxamide

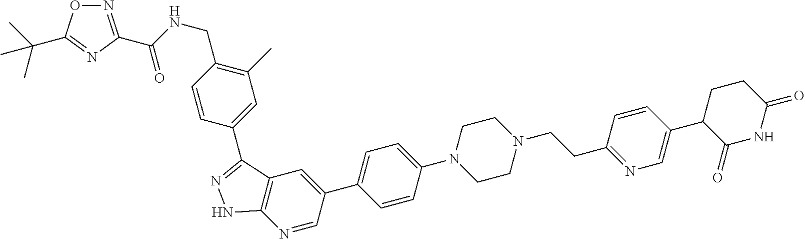 [SMILES: Cc1cc(-c2n[nH]c3ncc(-c4ccc(N5CCN(CCc6ccc(C7CCC(=O)NC7=O)cn6)CC5)cc4)cc23)ccc1CNC(=O)c1noc(C(C)(C)C)n1]

### Step 1: 2-(5-(4,4,5,5-tetramethyl-1,3,2-dioxaborolan-2-yl)pyridin-2-yl)ethan-1-ol

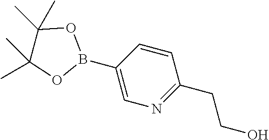 [SMILES: CC1(C)OB(c2ccc(CCO)nc2)OC1(C)C]

A mixture of 2-(5-bromopyridin-2-yl)ethan-1-ol (5 g, 24.9 mmol), bis(pinacolato)diboron (7.55 g, 29.8 mmol), Pd(dppf)Cl₂ (3.64 g, 4.97 mmol) and AcOK (4.9 g, 49.7 mmol) in dioxane (100 mL) was stirred at 100° C. for 16 h under a nitrogen atmosphere. The mixture was filtered and the filtrate was used in the next step directly. [M+H]⁺=250.1.

### Step 2: 2-(2′,6′-bis(benzyloxy)-[3,3′-bipyridin]-6-yl)ethan-1-ol

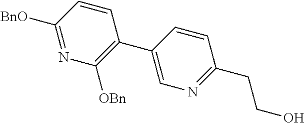 [SMILES: OCCc1ccc(-c2ccc(OCc3ccccc3)nc2OCc2ccccc2)cn1]

To a mixture of 2-(5-(4,4,5,5-tetramethyl-1,3,2-dioxaborolan-2-yl)pyridin-2-yl)ethan-1-ol (6 g, 24 mmol), 2,6-bis(benzyloxy)-3-bromopyridine (5.9 g, 16 mmol), Pd(dppf)Cl₂ (1.17 g, 1.6 mmol) and Cs₂CO₃ (10.4 g, 32 mmol) in dioxane (70 mL) and H₂O (20 mL) was stirred at 100° C. for 16 h under a nitrogen atmosphere. The mixture was concentrated under vacuum. The residue was purified with silica gel column chromatography (EtOAc/PE to 100% EtOAc, gradient elution) to give the product (4.0 g, 40.4%). [M+H]⁺=413.2.

### Step 3: 3-(6-(2-hydroxyethyl)pyridin-3-yl)piperidine-2,6-dione

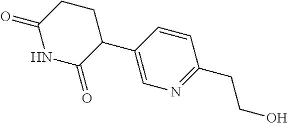 [SMILES: O=C1CCC(c2ccc(CCO)nc2)C(=O)N1]

A mixture of 2-(2′,6′-bis(benzyloxy)-[3,3′-bipyridin]-6-yl)ethan-1-ol (3.8 g, 9.2 mmol) in MeOH (40 mL) was added Pd/C (1.5 g, 10%). The mixture was stirred at room temperature for 16 h under a hydrogen atmosphere. The mixture was filtered and the filtrate was concentrated under vacuum. To the residue was added MeOH (1 mL) and Et₂O (40 mL). The mixture was stirred at room temperature for 10 min, filtered and solids were collected and dried under vacuum to afford the product (1.33 g, 61.3%). [M+H]⁺=235.1.

### Step 4: 2-(5-(2,6-dioxopiperidin-3-yl)pyridin-2-yl)ethyl 4-methylbenzenesulfonate

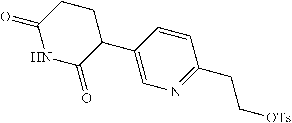 [SMILES: Cc1ccc(S(=O)(=O)OCCc2ccc(C3CCC(=O)NC3=O)cn2)cc1]

To a solution of 3-(6-(2-hydroxyethyl)pyridin-3-yl)piperidine-2,6-dione (300 mg, 1.28 mmol) in pyridine (10 mL) was added TsCl (487.2 mg, 2.56 mmol). The mixture was stirred at room temperature for 16 h and concentrated under vacuum. The residue was purified with silica gel column chromatography (MeOH/DCM, 0% to 15%, gradient elution) to give the product (140 mg, 28.2%). [M+H]⁺=389.1.

### Step 5: 5-(tert-butyl)-N-(4-(5-(4-(4-(2-(5-(2,6-dioxopiperidin-3-yl)pyridin-2-yl)ethyl)piperazin-1-yl)phenyl)-1H-pyrazolo[3,4-b]pyridin-3-yl)-2-methylbenzyl)-1,2,4-oxadiazole-3-carboxamide

A mixture of 5-(tert-butyl)-N-(2-methyl-4-(5-(4-(piperazin-1-yl)phenyl)-1H-pyrazolo[3,4-b]pyridin-3-yl)benzyl)-1,2,4-oxadiazole-3-carboxamide hydrochloride (106 mg, 0.18 mmol), 2-(5-(2,6-dioxopiperidin-3-yl)pyridin-2-yl)ethyl 4-methylbenzenesulfonate (70 mg, 0.18 mmol), DIEA (139.3 mg, 1.08 mmol) and KI (179.3 mg, 108 mmol) in MeCN (10 mL) and DMF (2 mL) was stirred at 80° C. for 16 h. The mixture was concentrated under vacuum. The residue was purified by Prep-HPLC to afford the desired product (27.7 mg, 20%). ¹H NMR (400 MHz, DMSO) δ_{H} 13.78 (s, 1H), 10.90 (s, 1H), 9.46 (s, 1H), 8.82 (s, 1H), 8.58 (s, 1H), 8.36 (s, 1H), 7.94-7.86 (m, 2H), 7.69 (d, J=8.0 Hz, 2H), 7.58 (d, J=8.0 Hz, 1H), 7.40 (d, J=8.0 Hz, 1H), 7.30 (d, J=8.0 Hz, 1H), 7.07 (d, J=8.0 Hz, 2H), 4.57-4.48 (m, 2H), 3.96-3.87 (m, 1H), 3.27-3.17 (m, 4H), 2.98-2.89 (m, 2H), 2.78-2.54 (m, 8H), 2.45 (s, 3H), 2.34-2.20 (m, 1H), 2.07-1.97 (m, 1H), 1.43 (s, 9H); [M+H]⁺=767.4.

Cell Degradation

Cell Treatment

TMD-8 cells are seeded at 20000 cells/well at a volume of 15 μl/well in cell culture medium [RPMI1640(Gibco, phenol red free, Cat #11835-030), 10% heat-inactive FBS, 1% PS(Gibco, Cat #10378)] in Corning 96 well plate (Cat #3799). TMD-8 cells are treated with compounds diluted in 0.2% DMSO, dilution is done according to the following protocol: (1) make 500× stock solution in DMSO from 1 mM by 6-fold dilution, total 8 doses were included; (2) make 2× solution in cell culture medium by transferring 0.5 μl 500× stock solution into 125 μl medium; (3) 15 μl of 2× solution is added to cells and incubate for 6 h.

HTFR Assay

After 6 h treatment, add 10 μl 4×lysis buffer to each well; seal the plate and incubate 30 min at room temperature on a plate shaker; Once the cells are lysed, 16 μL of cell lysate are transferred to a PE 384-well HTRF detection plate; 4 μL of pre-mixed HTRF antibodies are added to each well; Cover the plate with a plate sealer, spin 1000 rpm for 1 min, Incubate overnight at room temperature; Read on BMG PheraStar with HTRF protocol (337 nm-665 nm-620 nm).

The inhibition (degradation) percentage of the compound was calculated by the following equation: Inhibition percentage of Compound=100−100×(Signal-low control)/(High control-low control), wherein signal=each test compound group
Low control=only lysis buffer without cells, indicating that BTK is completely degraded;
High control=Cell group with added DMSO and without compound, indicating microplate readings without BTK degradation;
Dmax is the maximum percentage of inhibition (degradation).

The IC₅₀ (DC₅₀) value of a compound can be obtained by fitting the following equation

Y=Bottom+(TOP−Bottom)/(1+((IC₅₀/X)\hat{ }hillslope))

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 1
Degradation results

| Exam- | DC50 | Dmax | Exam- | DC50 | Exam- | DC50 |
|---|---|---|---|---|---|---|
| ple | (nM) | (%) | ple | (nM) | ple | (nM) |
| 1 | 0.978 | 96.73 | 96 | >2000.0 | 166 | 3.07 |
| 2 | 0.953 | 96.15 | 97 | 5.8 | 167 | 4.13 |
| 3 | 0.714 | 97.07 | 98 | 2.68 | 168 | 15.56 |
| 4 | 1.19 | 96.65 | 99 | 17.83 | 169 | 15.1 |
| 5 | 1.55 | 96.98 | 100 | 10.2 | 170 | 4.77 |
| 6 | 2.94 | 97.02 | 101 | 0.959 | 171 | 3.52 |
| 7 | 1.81 | 98.17 | 102 | 9.72 | 172 | 20.77 |
| 8 | 1.4 | 100.53 | 103 | 1.31 | 173 | >2000.0 |
| 9 | 1.9 | 100.45 | 104 | >2000.0 | 174 | 1.68 |
| 10 | 10.85 | 97.13 | 105 | 3.55 | 175 | 8.39 |
| 11 | 5.98 | 94.12 | 106 | 5.61 | 176 | 1.89 |
| 12 | 5.17 | 95.65 | 107 | 1.89 | 177 | 3.47 |
| 13 | 25.7 | 92.98 | 108 | 4.13 | 178 | 6.81 |
| 14 | 4.74 | 95.24 | 109 | 13.27 | 179 | 6.28 |
| 15 | 6.38 | 94.93 | 111 | 4.93 | 180 | 4.81 |
| 16 | 9.04 | 96.42 | 112 | 1.79 | 181 | 1.24 |
| 17 | 5.9 | 96.36 | 113 | 17.01 | 182 | 11.84 |
| 18 | >2000.0 | 3.39 | 114 | 1.56 | 183 | 19.84 |
| 19 | 16.86 | 92.84 | 115 | 4.87 | 184 | 13.95 |
| 20 | 3.95 | 96.44 | 116 | 1.05 | 185 | 16.24 |
| 21 | 8.62 | 97.5 | 117 | 3.5 | 186 | 1.61 |
| 22 | 9.1 | 97.6 | 118 | 8.07 | 187 | 9.56 |
| 23 | 4.04 | 96.04 | 119 | 2.12 | 188 | 9.91 |
| 24 | 4.62 | 97.32 | 120 | 5.82 | 189 | 23.6 |
| 25 | 8.16 | 96.68 | 121 | 2.39 | 190 | 6.11 |
| 26 | 28.93 | 94.35 | 122 | 2.22 | 191 | 7.34 |
| 27 | 9.25 | 95.27 | 123 | 14.25 | 192 | 4.03 |
| 28 | 2.54 | 97.06 | 124 | 1.42 | 193 | 3.06 |
| 29 | 3.2 | 95.44 | 125 | 15.22 | 194 | 1.69 |
| 30 | 8.19 | 97.55 | 127 | 6.79 | 195 | 6.13 |
| 31 | 21.84 | 97.4 | 128 | 5.84 | 196 | 1.05 |
| 32 | 2.55 | 93.63 | 129 | 3.73 | 197 | 1.08 |
| 33 | 3.85 | 99.39 | 130 | 5.28 | 198 | 91.42 |
| 34 | 3.39 | 95.57 | 131 | 5.9 | 199 | 5.09 |
| 35 | 1.9 | 93.02 | 132 | >2000.0 | 200 | >2000.0 |
| 36 | 3 | 98.3 | 133 | 1.96 | 201 | 1.79 |
| 37 | 2.52 | 93.42 | 134 | 9.96 | 202 | 9.04 |
| 38 | 3.2 | / | 135 | 12.74 | 203 | 5.27 |
| 39 | 2.6 | / | 136 | 14.9 | 204 | 2.02 |
| 40 | 7.32 | / | 137 | 5.23 | 205 | 0.736 |
| 41 | 7.05 | / | 138 | 8.6 | 206 | >2000.0 |
| 42 | 22.8 | / | 139 | 1.03 | 207 | 53.97 |
| 43 | 3.44 | / | 140 | 1.11 | 208 | 2.08 |
| 44 | 6.02 | / | 141 | 3.27 | 209 | 13.59 |
| 45 | 2.15 | / | 142 | 6.15 | 210 | 5.98 |
| 46 | 5.13 | / | 143 | 18.77 | 211 | 13.51 |
| 48 | 4.76 | / | 144 | 1.93 | 212 | 1.95 |
| 50 | 4.65 | / | 145 | 1.22 | 213 | 5.04 |
| 51 | 3.83 | / | 146 | 1.74 | 214 | 1.3 |
| 63 | 4.22 | / | 147 | 2.11 | 215 | 11.83 |
| 65 | 7.67 | / | 148 | 1.84 | 216 | 10.51 |
| 66 | 5.32 | / | 149 | 1.6 | 217 | 1962.7 |
| 68 | 1.47 | / | 150 | 8.88 | 218 | 18.96 |
| 69 | 2.5 | / | 151 | 17.79 | 219 | 2.23 |
| 71 | 4.03 | / | 152 | 1.03 | 220 | 3.85 |
| 72 | 1.21 | / | 153 | 11.8 | 221 | 1.12 |
| 69 | 2.37 | / | 154 | 3.27 | 222 | 16.13 |
| 78 | 1.75 | / | 155 | 2.92 | 223 | 1.56 |
| 86 | 3.95 | / | 156 | 2.14 | 224 | 6.16 |
| 87 | 7.21 | / | 157 | 62.14 | 225 | 9.45 |
| 88 | 9.41 | / | 158 | 2.07 | 226 | 3.04 |
| 89 | 0.635 | / | 159 | 56.35 | 227 | 3.93 |
| 90 | >2000.0 | / | 160 | 10.62 | 228 | 2.02 |
| 91 | 1.97 | / | 161 | 5.27 | 229 | 0.427 |
| 92 | 7.24 | / | 162 | 1.01 | | |
| 93 | 28.76 | / | 163 | 2.13 | | |
| 94 | 13.08 | / | 164 | 2.05 | | |
| 95 | 71.44 | / | 165 | 2.7 | | |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

What is claimed is:

1. A compound of Formula (I): 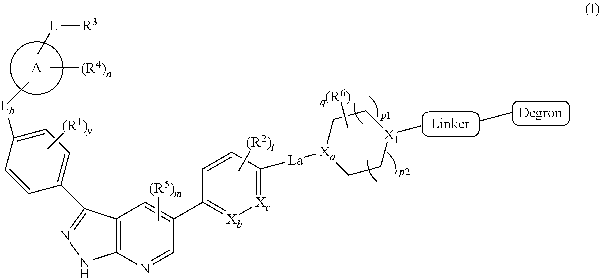
or a pharmaceutically acceptable salt thereof, or a stereoisomer thereof,
wherein:
A is a 5- or 6-membered aromatic ring comprising 0-3 heteroatoms selected from nitrogen, oxygen and sulfur;
X₁ and Xₐ are each selected from —CH— or N;
X_{b} and X_{c} are each selected from —CR^{a}— or N;
L and L_{b}, are each independently a bond, —(CR^{a}R^{b})ᵤ₁—, —NR⁷—, —O—, —S—, —(CR^{a}R^{b})ᵤ₁—NR⁷—C(O)—, —C(O)—NR⁷—(CR^{a}R^{b})ᵤ₁—, and Lₐ is a bond, —(CR^{a}R^{b})ᵤ₁—, —NR⁷—, —O—, —S—, —(CR^{a}R^{b})ᵤ₁—NR⁷—C(O)—, —C(O)—NR⁷—(CR^{a}R^{b})ᵤ₁—, 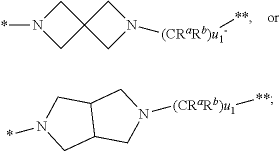
wherein u1 is an integral of 0-12; wherein * refers to the position attached to the 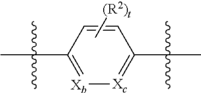
moiety, and ** refers to the position attached to the 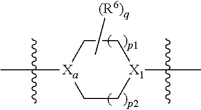
moiety;
t, m, n, q, and y are each independently 0, 1, 2, 3 or 4;
p1 and p2 are each independently 0, 1 or 2;
R⁷ is each independently hydrogen, —C₁₋₈alkyl, —C₂₋₈alkenyl, —C₂₋₈alkynyl, cycloalkyl, heterocyclyl, aryl, or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
R¹, R², R³, R⁴, R⁵, and R⁶ are each independently hydrogen, halogen, —C₁₋₈alkyl, —C₂₋₈alkenyl, —C₂₋₈alkynyl, cycloalkyl, heterocyclyl, aryl, heteroaryl, —CN, —NO₂, —OR^{a}, —SO₂R^{a}, —COR^{a}, —CO₂R^{a}, —CONR^{a}R^{b}, —C(═NR^{a})NR^{b}R^{c}, —NR^{a}R^{b}, —NR^{a}COR^{b}, —NR^{a}CONR^{b}R^{c}, —NR^{a}CO₂R^{b}, —NR^{a}SONR^{b}R^{c}, —NR^{a}SO₂NR^{b}R^{c}, or —NR^{a}SO₂R^{b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, hydroxyl-C₁₋₈alkyl-, -haloC₁₋₈alkyl, —C₁₋₈alkyoxy, cycloalkyl, heterocyclyl, aryl, or heteroaryl;
or in the case that two substituent R⁶ are substituted on the 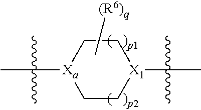
moiety, two R⁶ together with the remainder of the moiety form a fused ring or a bridged ring wherein the bridge comprises one, two, three or four —CH₂— moieties in addition to the two bridgeheads;
R^{a}, R^{b}, and R^{c} are each independently hydrogen, —C₁₋₈alkyl, —C₂₋₈alkenyl, —C₂₋₈alkynyl, cycloalkyl, heterocyclyl, aryl, or heteroaryl;
or R^{a} and R^{b}, together with the nitrogen atom to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{3f}, —SO₂R^{3f}, —SO₂NR^{3f}R^{3g}, —COR^{3f}, —CO₂R^{3f}, —CONR^{3f}R^{3g}, —C(═NR^{3f})NR^{3g}R^{3h}, —NR^{3f}R^{3g}, —NR^{3f}COR^{3g}, —NR^{3f}CONR^{3g}R^{3h}, —NR^{3f}CO₂R^{3g}, —NR^{3f}SONR^{3g}R^{3h}, —NR³SO₂NR^{3g}R^{3h}, or —NR^{3f}SO₂R^{3g}, each of said —C₁₋₈alkyl, —C₂₋₈alkenyl, —C₂₋₈alkynyl, cycloalkyl, heterocyclyl, aryl, or heteroaryl is optionally substituted with at least one substituent selected from halogen, —C₁₋₈alkyl, —OR³ⁱ, —NR³ⁱR^{3j}, cycloalkyl, heterocyclyl, aryl, or heteroaryl;
R^{3f}, R^{3g}, R^{3h}, R³ⁱ, and R^{3j} are each independently hydrogen, —C₁₋₈alkyl, C₁₋₈alkoxy-C₁₋₈alkyl-, —C₂₋₈alkenyl, —C₂₋₈alkynyl, cycloalkyl, heterocyclyl, aryl, or heteroaryl;
the Linker is a bond or a divalent linking group, and
the Degron moiety is an E3 Ubiquitin ligase moiety.

2. The compound according to claim 1, wherein the Degron moiety is selected from Formulas D1, D2, D3, D4, D5, D6, D7 or D8: 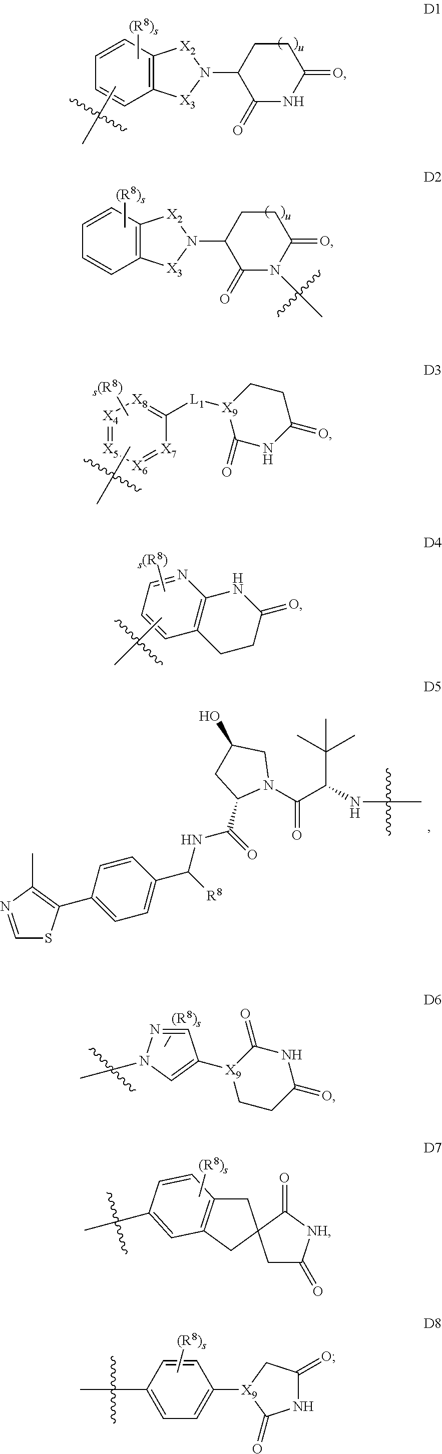
wherein
X₂ and X₃ are each independently —CH₂—, —NH— or —C(O)—;
X₄, X₅, X₆, X₇ and X₈ are each independently CH or N;
X₉ is CH or N;
L₁ is selected from a bond, —CH₂—, —O—, —NH— and —S—;
s is 0, 1, 2, 3, or 4;
u is 0, 1, or 2;
R⁸ is each independently hydrogen, halogen, —C₁₋₈alkyl, —C₂₋₈alkenyl, —C₂₋₈alkynyl, cycloalkyl, heterocyclyl, aryl, heteroaryl, —CN, —NO₂, —OR^{8a}, —SO₂R^{8a}, —COR^{8a}, —CO₂R^{8a}, —CONR^{8a}R^{8b}, —C(═NR^{8a})NR^{8b}R^{8c}, —NR^{8a}R^{8b}, —NR^{8a}COR^{8b}, —NR^{8a}CONR^{8b}R^{8c}, —NR^{8a}CO₂R^{8b}, —NR^{8a}SONR^{8b}R^{8c}, —NR^{8a}SO₂NR^{8b}R^{8c}, or —NR^{8a}SO₂R^{8b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
R^{8a}, R^{8b}, and R^{8c} are each independently hydrogen, —C₁₋₈alkyl, —C₂₋₈alkenyl, —C₂₋₈alkynyl, cycloalkyl, heterocyclyl, aryl, or heteroaryl;
Alternatively, two adjacent R⁸, together with the ring to which they are attached, form a fused ring;
wherein the Degron moiety binds to the linker via

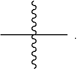

3. The compound according to claim 1, wherein Formula D1 is selected from

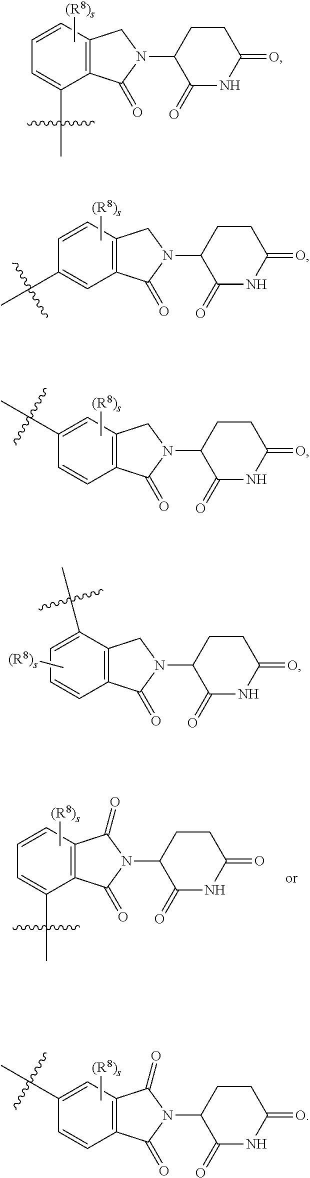

4. The compound according to claim 1, wherein Formula D1 or D2 is selected from

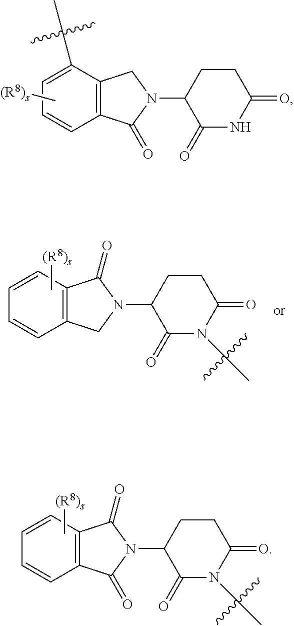

5. The compound according to claim 4, wherein Formula D1 or D2 is selected from

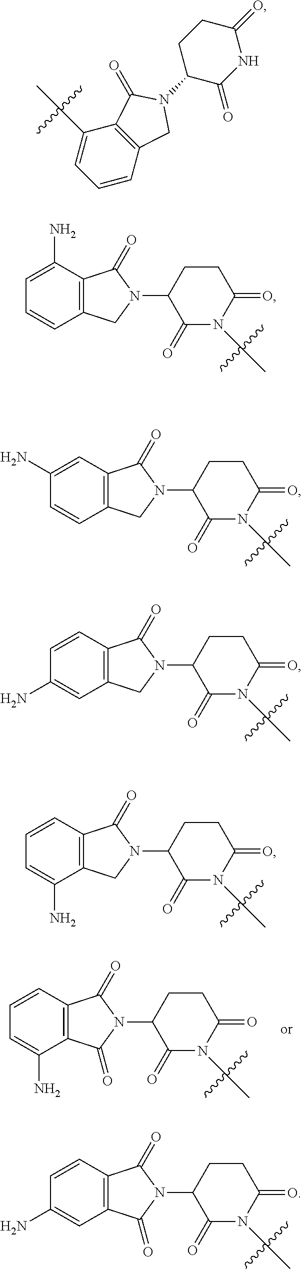

6. The compound according to claim 2, wherein Formula D3, D6 or D8 is selected from 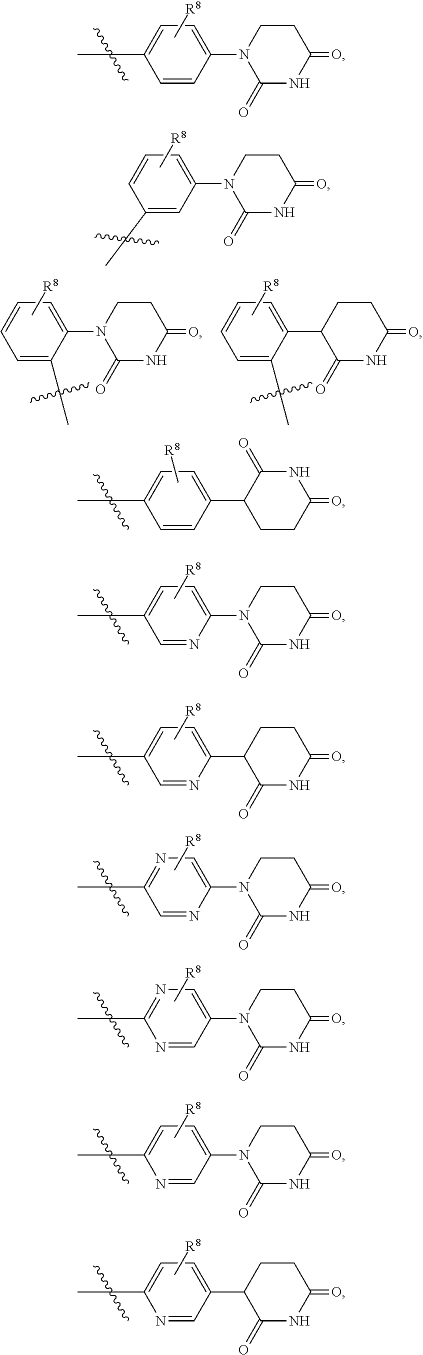 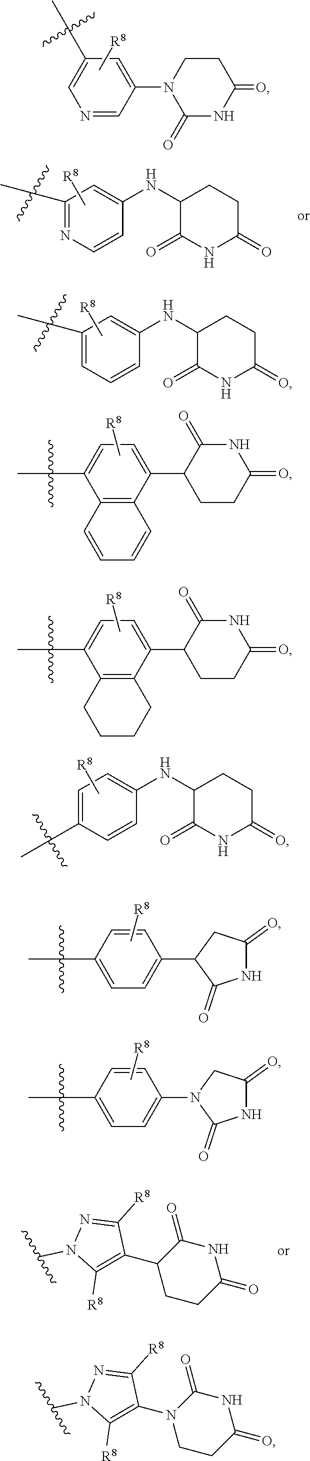
wherein R⁸ is defined as above.

7. The compound according to claim 6, wherein Formula D3, D6 or D8 is selected from 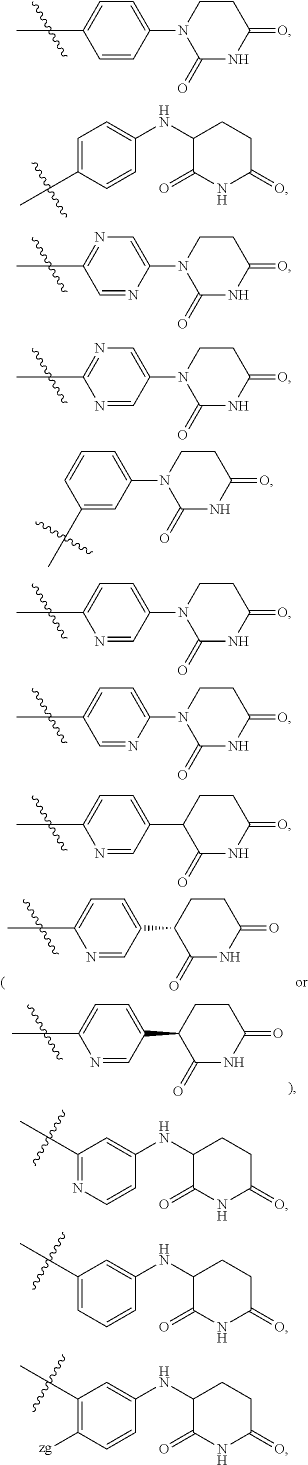 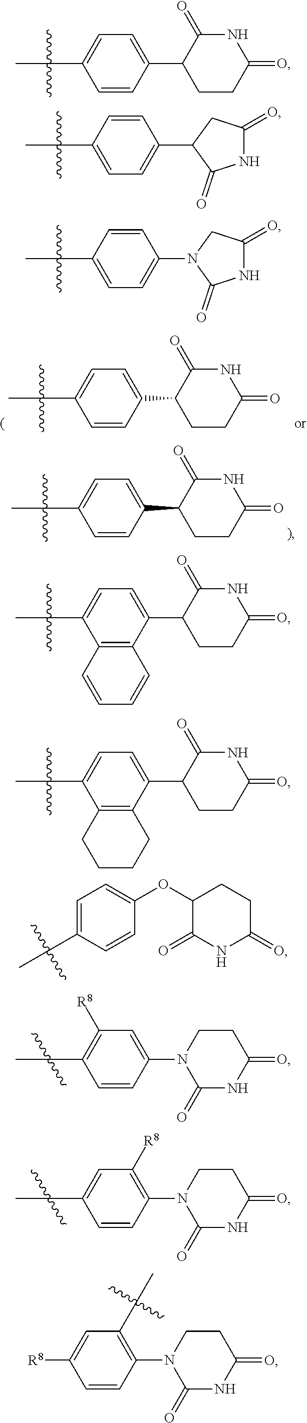 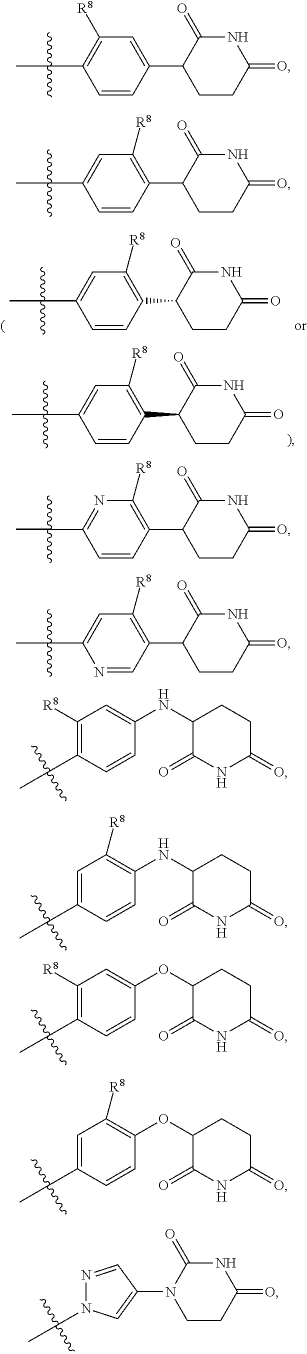 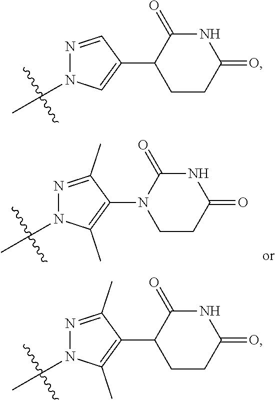
wherein R⁸ is halogen, —C₁₋₈alkyl, or —C₁₋₈alkoxy.

8. The compound according to claim 2, wherein Formula D4 is selected from

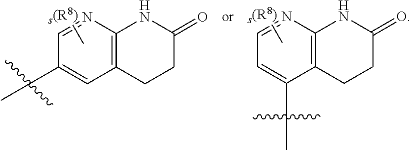

9. The compound according to claim 8, wherein Formula D4 is selected from

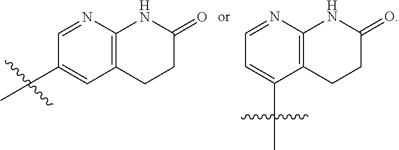

10. The compound according to any one of claims 1-9, wherein the Linker is selected from a bond, 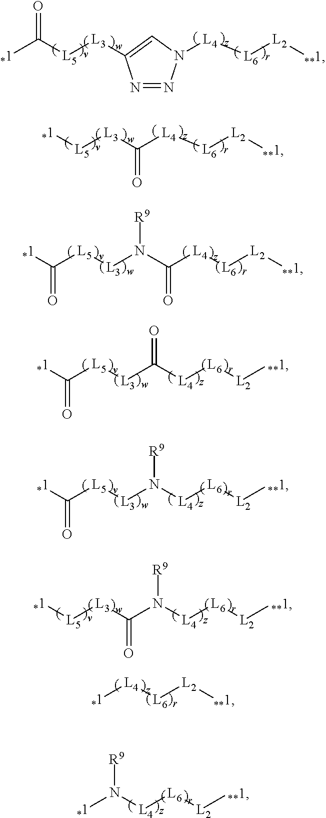
wherein *1 refers to the position attached to the 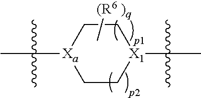
moiety, and **1 refers to the position attached to the Degron;
r, v, w, and z are each independently 0, 1, 2, 3, 4, 5, 6, 7, 8, 9 or 10;
L₂ is —CH₂—, —NH—, O—, —C(O)—, —NHC(O)—, 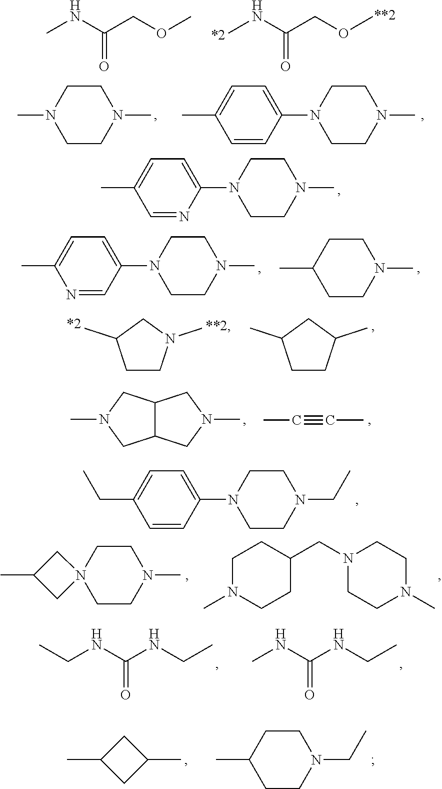
wherein *2 refers to the position attached to L₄ and **2 refers to the position attached to the Degron;
L₃, L₄, L₅ and L₆ are each independently —CH₂—, —CH₂—CH(CH₃)—, —CF₂—, —CH₂CH₂—, —OCH₂CH₂—, —CH₂—O—CH₂—, —CH₂CH₂O—, —C(O)—, —NHC(O)—, —CH₂—CONH—, 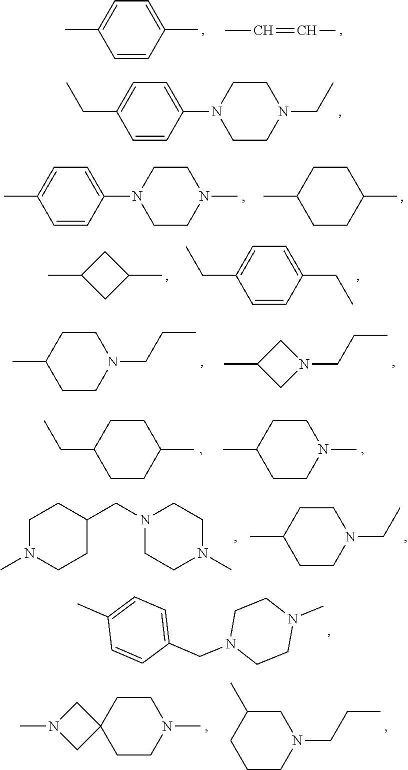
R⁹ is selected from H or CH₃.

11. The compound according to any one of claims 1-10, wherein the Linker is selected from 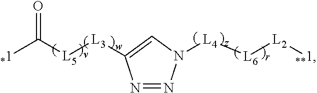
v=0; w=0, 1, 2, 3, or 4; L₃ is —CH₂—; L₄ is —CH₂CH₂O— or —CH₂—; z=0, 1, 2, 3, 4, 5, 6, or 7; L₆ is —CH₂— or —NHC(O)—; r=0, 1, 2, 3, or 4; L₂ is —NH—, —CH₂—, —O— or —C≡C—.

12. The compound according to claim 10, wherein v=0; w=0; L₄ is —CH₂CH₂O—; z=1, 2, 3, 4, 5, 6, or 7; L₆ is —CH₂—; r=0, 1, 2, or 3; L₂ is —NH—, or —CH₂—.

13. The compound according to claim 11, wherein v=0; w=0; L₄ is —CH₂CH₂O—; z=1, 2, 3; L₆ is —CH₂—; r=1, 2, or 3; L₂ is —C≡C—.

14. The compound according to claim 10, wherein v=0, L₃ is —CH₂—, w=2 or 3, L₄ is —CH₂CH₂O— or —CH₂—, z=1, 2, 3, or 4; L₆ is —CH₂—; r=1, 2, or 3; L₂ is —NH—, or —CH₂—.

15. The compound according to claim 10, wherein v=0, L₃ is —CH₂—, w=2 or 3, L₄ is —CH₂—, z=3, 4 or 5; r=0; L₂ is 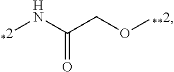
wherein *2 refers to the position attached to L₄ and **2 refers to the position attached to the Degron.

16. The compound according to claim 10, wherein the Linker is selected from 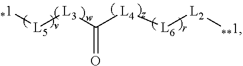
wherein
L₅ is —CH₂CH₂O—, or 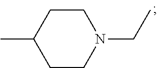
v=0, 1, 2 or 3, L₃ is —CH₂— or 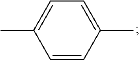
w=0, 1, 2, or 3; L₄ is —CH₂—O—CH₂—, —CH₂—, 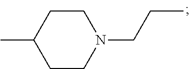
z=0, 1, 2, 3, 4, 5, or 6; L₆ is —CH₂—, —OCH₂CH₂—, 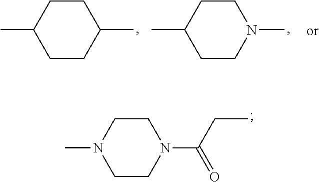
r=0, 1, 2, 3, 4, 5, 6, 7 or 8; L₂ is —NH—,

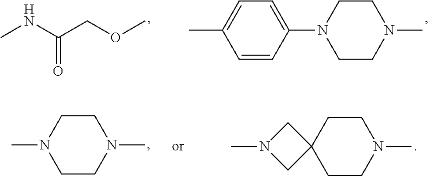

17. The compound according to claim 16, wherein L₅ is —CH₂CH₂O—; v=1, 2 or 3, L₃ is —CH₂—; w=1; z=0; r=0; L₂ is —NH—.

18. The compound according to claim 16, wherein v=w=0; L₄ is —CH₂—O—CH₂—; z=1, 2, 3 or 4; L₆ is —CH₂—; r=1, 2, 3, 4, 5, 6, 7 or 8; L₂ is —NH— or

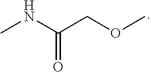

19. The compound according to claim 16, wherein v=w=z=0; L₆ is —CH₂—; r=2, 3, 4, 5, or 6;
L₂ is —NH— or

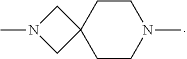

20. The compound according to claim 16, wherein v=w=0; L₄ is 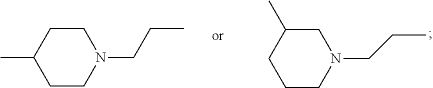
z=1; L₆ is —OCH₂CH₂—; r=1, 2, 3; L₂ is —NH—.

21. The compound according to claim 16, wherein the Linker is selected from 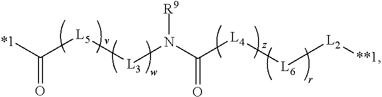
wherein
L₅ is —CH₂CH₂O—, —CH₂— or —CH₂—O—CH₂—;
v=1, 2, 3 or 4, L₃ is —CH₂—, 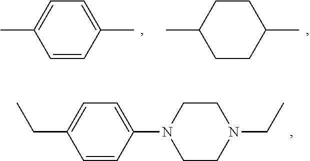
or —CH₂CH₂O—;
w=0, 1, 2 or 3;
R⁹ is H or CH₃;
L₄ is —CH₂— or —CH₂—O—CH₂—;
z=0, 1, 2, 3, or 4; L₆ is —CH₂—;
r=0, 1, 2, 3, or 4; L₂ is —NH—, —CH₂—, —O—,

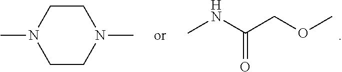

22. The compound according to claim 21, wherein
L₅ is —CH₂—;
v=1, 2, 3 or 4,
L₃ is 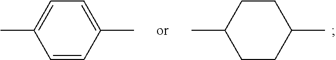
w=1;
R⁹ is H;
L₄ is —CH₂—;
z=1; r=0; L₂ is —NH—.

23. The compound according to claim 10, wherein the Linker is selected from 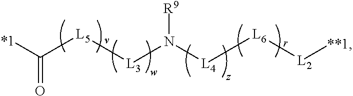
wherein
L₅ is —CH₂CH₂O—, —CH₂—, —CH═CH—, or —CH₂—O—CH₂—;
v=1, 2, 3 or 4, L₃ is —CH₂—, —C(O)— 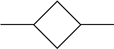
or —CH₂CH₂O—;
w=0, 1, 2 or 3; R⁹ is H or CH₃; L₄ is —CH₂CH₂O—, —CH₂—, —CH₂—O—CH₂—, —CH₂—CONH— or 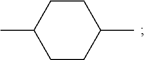
z=0, 1, 2, 3, 4, or 5; L₆ is —CH₂—, 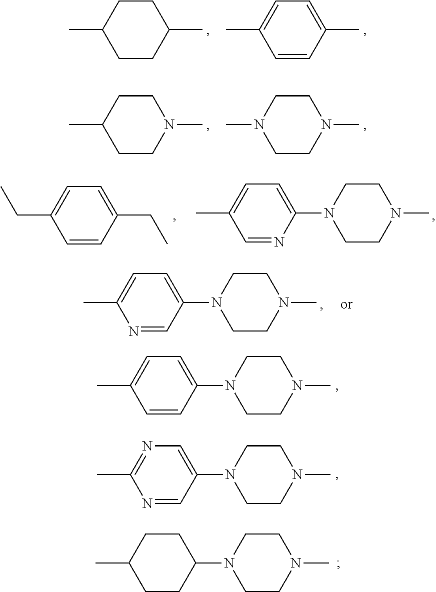
r=0, 1, 2, 3, 4, 5, or 6; L₂ is —NH—, —C(O)—, —O—,

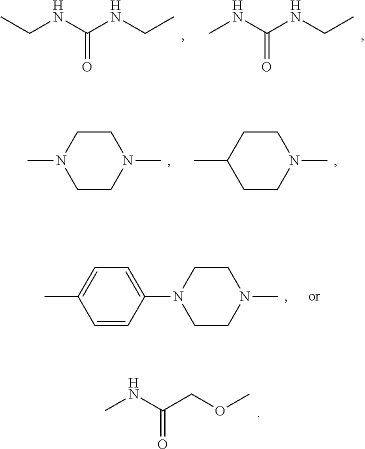

24. The compound according to claim 23, wherein L₅ is —CH₂—; v=2; w=0; R⁹ is CH₃; L₄ is —CH₂—; z=1, 2, 3, or 4; L₆ is —CH₂—, 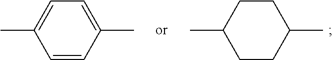
r=0, 1 or 2; L₂ is —NH—,

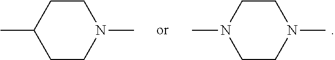

25. The compound according to claim 23, wherein L₅ is —CH═CH—; v=1, L₃ is —CH₂—; w=0 or 1; R⁹ is H or CH₃; L₄ is —CH₂CH₂O— or —CH₂—; z=1, 2, 3, 4, 5 or 6; L₆ is —CH₂—; r=0, 1, 2; L₂ is —NH—, —CH₂—, or

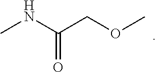

26. The compound according to claim 23, wherein v=0;
L₃ is 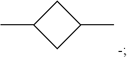
w=1;
R⁹ is CH₃;
L₄ is —CH₂—;
z=1 or 2;
L₆ is 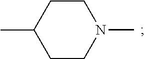
r=0 or 1;
L₂ is —NH—, 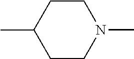
or —C(O)—.

27. The compound according to claim 10, wherein the Linker is selected from 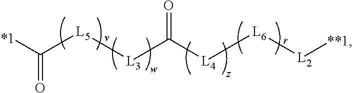
wherein
L₅ is —CH═CH—;
v=1, 2, 3 or 4;
L₃ is 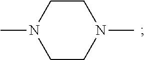
w=1;
L₄ is —CH₂—;
z=1, 2;
L₆ is —CH₂—;
r=0;
L₂ is —NH— or —CH₂—.

28. The compound according to claim 10, wherein the Linker is selected from 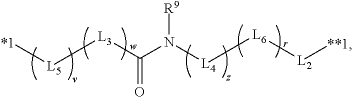
wherein
L₅ is 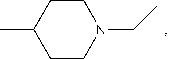
CH₂CH₂O—, —CH₂— or —CH₂—O—CH₂—;
v=1, 2, 3 or 4,
L₃ is —CH₂—, 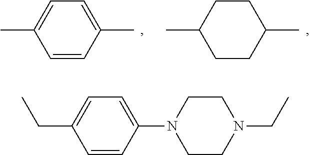
or —CH₂CH₂O—;
w=0, 1, 2 or 3;
R⁹ is H or CH₃;
L₄ is —CH₂—, —CH₂—O—CH₂—, 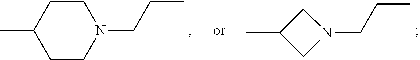
z=0, 1, 2, 3, or 4;
L₆ is —CH₂— or —OCH₂CH₂—;
r=0, 1, 2, 3, or 4;
L₂ is —NH—, —CH₂—, —O—,

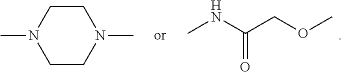

29. The compound according to claim 10, wherein the Linker is selected from 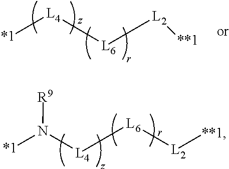
L₄ is —CH₂—, —CF₂—, 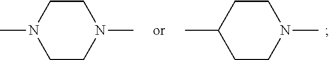
z=0, 1, 2, 3, 4, 5 or 6;
L₆ is —CH₂—, —CF₂—, —CHCH₃—, 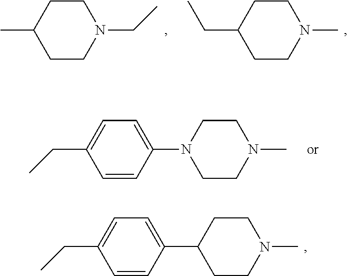
r=0 or 1;
L₂ is —O—, 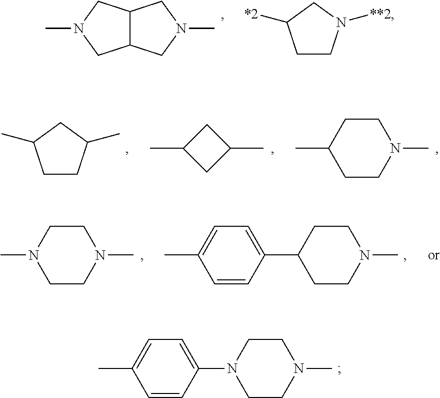
R⁹ is H or CH₃.

30. The compound according to claim 10, wherein the Linker is 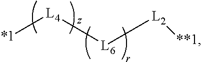
wherein L₄ is —CH₂— or —CF₂—; z=0, 1, 2, 3, 4, 5 or 6; r=0; and L₂ is —O—, —C(O)—,

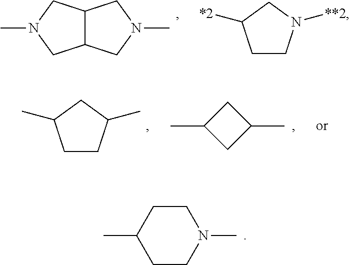

31. The compound according to claim 10, wherein 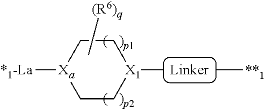
is selected from

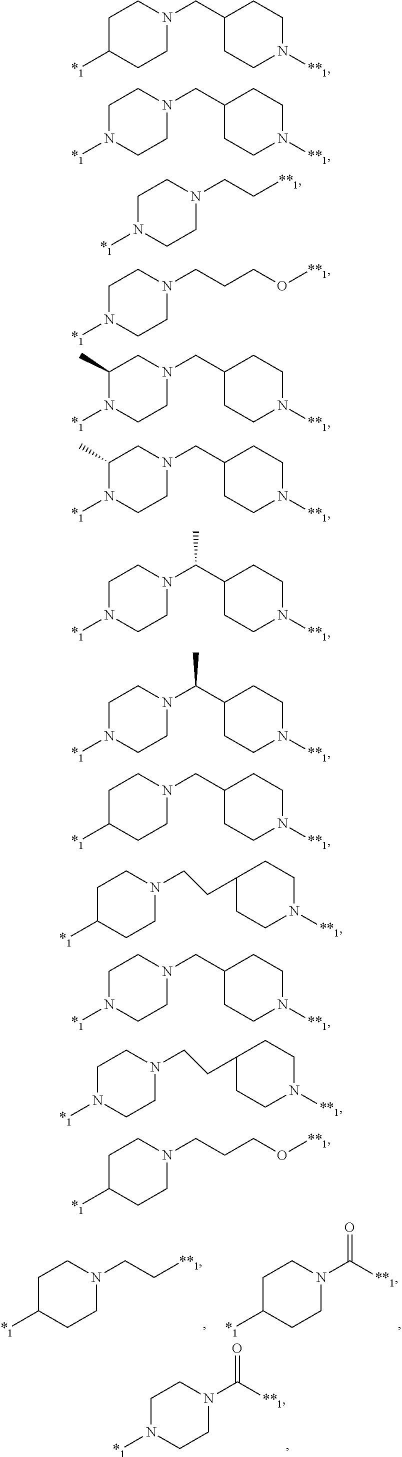

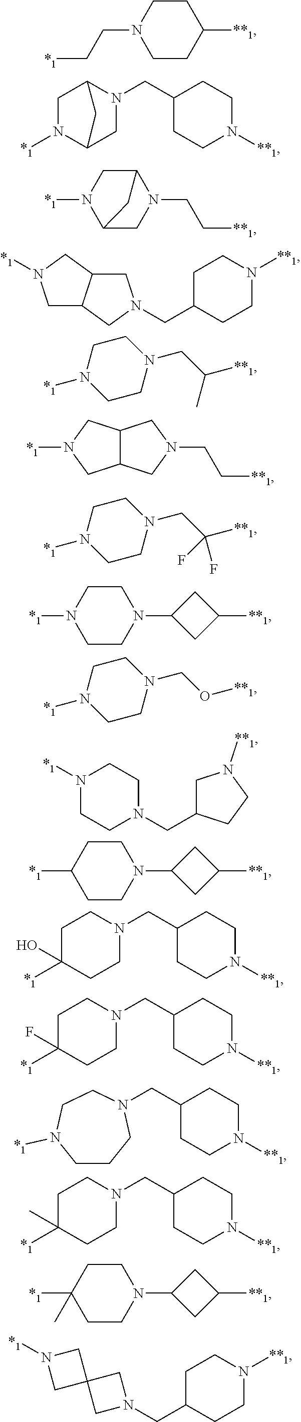

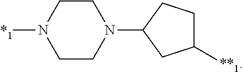

32. The compound according to claim 1, wherein ring A is 5-membered aromatic ring comprising 1-3 heteroatoms selected from nitrogen and oxygen.

33. The compound according to claim 32, wherein ring A is benzyl, oxadiazole, triazole, thiazole, or pyrazole.

34. The compound according to claim 1, wherein L_{b} is —(CR^{a}R^{b})ᵤ₁—NR⁷—C(O)—, or —C(O)—NR⁷—(CR^{a}R^{b})ᵤ₁—; wherein u1 is an integral of 0-12.

35. The compound according to claim 1, wherein y is 0 or 1 or 2, and R¹ is halogen or —C₁₋₈alkyl or hydroxyl-C₁₋₈alkyl-.

36. The compound according to claim 1, wherein X_{b} is CH and X_{c} is N; or X_{b} is N and X_{c} is CH; or X_{b} is CH and X_{c} is CH.

37. The compound according to claim 1, wherein X₁ is N and Xₐ is CH; or X₁ is N and Xₐ is N.

38. The compound according to claim 1, wherein the 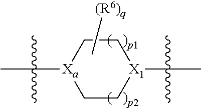
moiety is 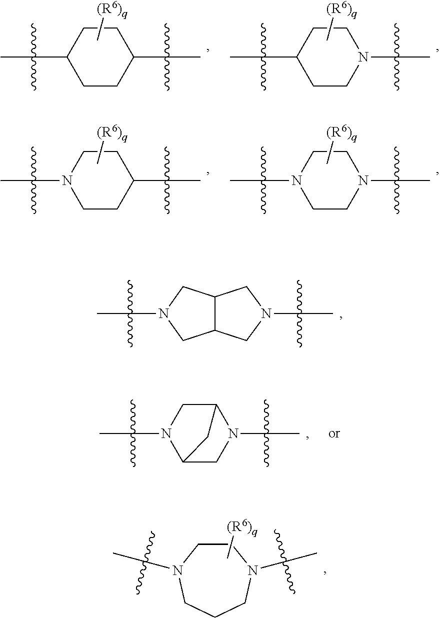
wherein R⁶ and q are defined as in Formula (I).

39. The compound according to claim 1, wherein the compound is selected from Examples 1 to 80, 86-109, 111-125, and 127-229.

40. A pharmaceutical composition comprising the compound according to any one of claims 1-39, or a pharmaceutically acceptable salt thereof, and at least one pharmaceutically acceptable carrier or excipient.

41. A method of decreasing BTK activity by inhibition and/or degradation, which comprises administering to an individual the compound according to any one of claims 1-39, or a pharmaceutically acceptable salt thereof, including the compound of formula (I) or the specific compounds exemplified herein.

42. A method of treating a disease or disorder in a patient comprising administering to the patient a therapeutically effective amount of the compound any one of claims 1-39, or a pharmaceutically acceptable salt thereof as a BTK kinase inhibitor and/or degrader, wherein the disease or disorder is associated with inhibition of BTK.
